(19) United States
(12) Patent Application Publication
(10) Pub. No.: US 2023/0051406 A1
(43) Pub. Date: Feb. 16, 2023

(54) GENETICALLY MODIFIED NATURAL KILLER CELLS AND METHODS OF USE THEREOF

(71) Applicant: Catamaran Bio, Inc., Cambridge, MA (US)

(72) Inventors: Vipin SURI, Belmont, MA (US); Bharat Duttala REDDY, Somerville, MA (US); Mark Ferris BOSHAR, Andover, MA (US); Celeste Jeanne RICHARDSON, Brookline, MA (US); Eugene Daehee CHOI, Arlington, MA (US); Meghan Elizabeth WALSH, Medford, MA (US); Jennifer Ann JOHNSON, Newburyport, MA (US)

(21) Appl. No.: 17/525,525

(22) Filed: Nov. 12, 2021

Related U.S. Application Data
(60) Provisional application No. 63/229,022, filed on Aug. 3, 2021.
(60) Provisional application No. 63/189,029, filed on May 14, 2021.
(60) Provisional application No. 63/143,180, filed on Jan. 29, 2021.
(60) Provisional application No. 63/113,318, filed on Nov. 13, 2020.

(51) Int. Cl. A61K 35/17; C12N 15/63; C12N 5/0783; A61P 35/00; C07K 14/54; C07K 14/55

(52) U.S. Cl. CPC A61K 35/17; C12N 15/63; C07K 14/55; A61P 35/00; C07K 14/5443; C12N 5/0646

(57) ABSTRACT

This disclosure describes genetically engineered natural killer (NK) cells, pharmaceutical compositions that include these NK cells, and methods of making and using these NK cells.

### CROSS-REFERENCE TO RELATED APPLICATION

This application claims priority to, and benefit of, U.S. Provisional Application No. 63/113,318, filed on Nov. 13, 2020; U.S. Provisional Application No. 63/143,180, filed on Jan. 29, 2021; U.S. Provisional Application No. 63/189,029, filed on May 14, 2021; and U.S. Provisional Application No. 63/229,022, filed on Aug. 3, 2021, the contents of which are incorporated by reference in their entirety.

### FIELD

The present disclosure relates generally to the fields of molecular biology, immunology, oncology and medicine. More particularly, it concerns natural killer cells expressing chimeric antigen receptors, such as chimeric antigen receptors that bind to a target protein.

### DESCRIPTION OF THE TEXT FILE SUBMITTED ELECTRONICALLY

The Sequence Listing associated with this application is provided in text format in lieu of a paper copy, and is hereby incorporated by reference into the specification. The name of the text file containing the Sequence Listing is “CATA-002_001US_SeqListing_ST25.txt”. The text file is 2,460 KB, was created on Nov. 12, 2021, and is being submitted electronically via EFS-Web.

### BACKGROUND

In recent years, adoptive cellular therapy using autologous T cells transduced to express chimeric antigen receptor (CARs) has proven to be a very powerful approach for the treatment of cancer, leading to U.S. Food and Drug Administration- (FDA) approved cell therapies for B cell leukemia and lymphoma. However, challenges remain, including uncoupling cytotoxicity against tumor cells from systemic toxicity, finding solutions for target antigen negative relapses, and developing universal off-the-shelf cell therapy products to avoid the logistic hurdles of generating autologous products, while managing the challenges of allogeneic T cell products, such as graft-versus-host disease (GVHD) (Hartmann et al. (2017) EMBO Mol. Med. 9:1183-97). Additional challenges of T cell therapies include the risk of cytokine release syndrome (CRS) and the difficulty of multifactorial engineering of T cell therapies that require both gene addition and deletion strategies.

Natural killer (NK) cells are attractive contenders since they mediate effective cytotoxicity against tumor cells and unlike T cells, lack the potential to cause GVHD in the allogeneic setting. Thus, NK cells could be made available as an off-the-shelf cellular therapy product for immediate clinical use (Daher et al. (2018) Curr. Opin. Immunol. 51: 146-153). Peripheral blood and cord blood are readily available sources of allogeneic NK cells with the potential for widespread clinical scalability. In addition, NK cells can also be obtained from differentiation of inducible pluripotent stem cells (iPSCs) or CD34⁺ hematopoietic stem cells (HSCs).

Cluster of Differentiation 70 (CD70, CD27LG or TNFSF7) is a member of the tumor necrosis factor (TNF) superfamily and is the membrane-bound ligand for CD27 receptor, which belongs to the TNF receptor superfamily (Hintzen et al. Int Immunol. 6(3): 477-80, 1994; Bowman et al. J Immunol. 152(4):1756-61, 1994). Physiologically, CD70 expression is transient and restricted to a subset of highly activated T cells, B cells, and dendritic cells. The transient interaction between CD27 and CD70 provides T cell costimulation complementary to that provided by CD28. Expression of CD70 is highly regulated and occurs in healthy individuals only transiently on activated T cells, antigen and Toll-like receptor-stimulated B cells, mature dendritic cells, NK cells and on dendritic and epithelial cells of the thymic medulla (Wajant et al. Expert Opin. Ther. Targets 20(8): 959-7 2016). CD70 is expressed in hematological cancers such as Acute Myeloid Leukemia (AML), Non-Hodgkin's Lymphoma, such as diffuse large B cell and follicular lymphoma and malignant cells of Hodgkin's lymphoma (Reed-Sternberg cells), Waldenstrom's macroglobulinemia and multiple myeloma, and by HTLV-1- and EBV-associated malignancies. (Agathanggelou et al. Am. J Pathol. 147(4):1152-60, 1995; Lens et al. Br J Haematol. 106(2): 491-503, 1999; Baba et al. J Virol. 82(8): 3843-52, 2008). In addition, CD70 is expressed by non-hematological malignancies such as renal cell carcinoma (RCC), small cell lung cancer (SCLC), pancreatic cancer, esophageal carcinoma, gastric carcinoma, mesothelioma, and glioblastoma (Dunker et al. J. Urol. 173(6): 2150-3, 2005; Chahlavi et al. Cancer Res. 65(12): 5428-38, 2005; Flieswasser et al. Cancers (Basel) 11(10):1611, 2019).

There is a need in the art for alternative approaches for generating genetically engineered NK cells that are useful as therapeutics. The present disclosure addresses this unmet need in the art.

### SUMMARY

Provided herein is a method of making a population of genetically engineered natural killer (NK) cells by: (a) contacting a population of NK cells with a CD70 inhibitor; and (b) expanding the population of NK cells in vitro.

In some embodiments, the population of NK cells is a population of human NK cells. In some embodiments, the population of NK cells exhibits at least about 25% greater cell expansion compared to a population of NK cells that is not contacted with the CD70 inhibitor. In some embodiments, the method further comprises, prior to step (a), isolating CD56⁺ cells and/or CD3⁻/CD56⁺ cells from a population of peripheral blood mononuclear cells (PBMCs) to obtain the population of NK cells.

In some embodiments, the expanding comprises culturing the population of NK cells in the presence of feeder cells. In certain embodiments, the feeder cells are an immortalized cell line. In other embodiments, the feeder cells are autologous feeder cells. In particular embodiments, the feeder cells have been irradiated.

In some embodiments, the expanding comprises culturing the population of NK cells in a culture medium comprising one or more of recombinant human IL-12, recombinant human IL-8, and recombinant human IL-21. In some embodiments, the expanding is performed from about 1 day to about 42 days.

In some embodiments, the CD70 inhibitor decreases the level of CD70 polypeptide in at least one NK cell of the population of NK cells. In some embodiments, the CD70 inhibitor comprises a small interfering RNA (siRNA) that targets CD70 mRNA, a short hairpin RNA (shRNA) that targets CD70 mRNA, a nucleic acid encoding a siRNA that targets CD70 mRNA, a nucleic acid encoding an shRNA that targets CD70 mRNA, a nucleic acid encoding a tandem shRNA that targets CD70 mRNA, a tandem shRNA that targets CD70 mRNA, a nucleic acid encoding a ribozyme that targets CD70 mRNA, or a ribozyme that targets CD70 mRNA, or a combination of any of the foregoing. In some embodiments, the CD70 inhibitor comprises an RNA-guided endonuclease and a guide RNA (gRNA) targeting a CD70 gene. In some embodiments, the CD70 inhibitor decreases cell surface level of CD70 polypeptide in at least one NK cell of the population of NK cells.

In some embodiments, the CD70 inhibitor comprises a Protein Expression Blocker (PEBL) or a nucleic acid encoding a PEBL, wherein the PEBL comprises a first antigen recognition domain that specifically binds human CD70 and one or more of a localizing domain, an intracellular retention domain and an endoplasmic reticulum (ER) retention domain.

In some embodiments, the CD70 inhibitor comprises an antagonistic anti-CD70 antibody or an antigen-binding fragment thereof. In certain embodiments, the antagonistic anti-CD70 antibody or the antigen-binding fragment thereof inhibits the interaction between CD70 and CD27. In particular embodiments, the antagonistic anti-CD70 antibody or the antigen-binding fragment thereof comprises a VH and a VL wherein a) the VH comprises SEQ ID NO: 1162 and the VL comprises SEQ ID NO: 1163; b) the VH comprises SEQ ID NO: 51 and the VL comprises SEQ ID NO: 53; c) the VH comprises SEQ ID NO: 11 and the VL comprises SEQ ID NO: 13; d) the VH comprises SEQ ID NO: 694 and the VL comprises SEQ ID NO: 69; e) the VH comprises SEQ ID NO: 1118 and the VL comprises SEQ ID NO: 1119; f) the VH comprises SEQ ID NO: 1120 and the VL comprises SEQ ID NO: 1121; g) the VH comprises SEQ ID NO: 1116 and the VL comprises SEQ ID NO: 1117; h) the VH comprises SEQ ID NO: 1104 and the VL comprises SEQ ID NO: 1105; i) the VH comprises SEQ ID NO: 1094 and the VL comprises SEQ ID NO: 1095; j) the VH comprises SEQ ID NO: 1084 and the VL comprises SEQ ID NO: 1085; k) the VH comprises SEQ ID NO: 1092 and the VL comprises SEQ ID NO: 1093; 1) the VH comprises SEQ ID NO: 1082 and the VL comprises SEQ ID NO: 1083; or m) the VH comprises SEQ ID NO: 1074 and the VL comprises SEQ ID NO: 1075. In specific embodiments, the antagonistic anti-CD70 antibody is cusatuzumab, MDX-1411, 27B3, 57B6, 59D10, 19G10, 9B2, 5B2, 9G2, 5F4, 9D1, and/or SGN70.

In some embodiments, the method further comprises (c) contacting the population of NK cells with a polynucleotide encoding a chimeric antigen receptor (CAR) under conditions sufficient to transfer the polynucleotide across a cell membrane of at least one NK cell in the population of NK cells, wherein the CAR comprises: (i) an extracellular domain comprising a second antigen recognition domain that specifically binds human CD70; (ii) a transmembrane domain; and (iii) an intracellular domain. In some embodiments, the CAR comprises an amino acid an amino acid sequence that is at least 90%, at least 91%, at least 92%, at least 93%, at least 94%, at least 95%, at least 96%, at least 97%, at least 98%, at least 99% or at least 100% identical to the amino acid sequence of SEQ ID NO: 637, 639, 641, 643, 645, 647, 700, 2561-2593, 2697-2736 or 2737-2882. In certain embodiments, the method further comprises expanding the population of NK cells in vitro after step (c).

In some embodiments of the aforementioned method, step (b) comprises expanding the population of NK cells by at least 1,000-fold in culture.

In some embodiments, the second antigen recognition domain comprises a scFv comprising a VH and a VL, wherein (a) the VH comprises a CDRH1 of SEQ ID NO: 86, a CDRH2 of SEQ ID NO: 87, and a CDRH3 of SEQ ID NO: 88, and the VL comprises a CDRL1 of SEQ ID NO: 89, a CDRL2 of SEQ ID NO: 90, and a CDRL3 of SEQ ID NO: 91; (b) the VH comprises a CDRH1 of SEQ ID NO: 25, a CDRH2 of SEQ ID NO: 26, and a CDRH3 of SEQ ID NO: 27, and the VL comprises a CDRL1 of SEQ ID NO: 28, a CDRL2 of SEQ ID NO: 29, and a CDRL3 of SEQ ID NO: 30; (c) the VH comprises a CDRH1 of SEQ ID NO: 35, a CDRH2 of SEQ ID NO: 36, and a CDRH3 of SEQ ID NO: 37, and the VL comprises a CDRL1 of SEQ ID NO: 38, a CDRL2 of SEQ ID NO: 39, and a CDRL3 of SEQ ID NO: 40; (d) the VH comprises a CDRH1 of SEQ ID NO: 45, a CDRH2 of SEQ ID NO: 46, and a CDRH3 of SEQ ID NO: 47, and the VL comprises a CDRL1 of SEQ ID NO: 48, a CDRL2 of SEQ ID NO: 49, and a CDRL3 of SEQ ID NO: 50; (e) the VH comprises a CDRH1 of SEQ ID NO: 55, a CDRH2 of SEQ ID NO: 56, and a CDRH3 of SEQ ID NO: 57, and the VL comprises a CDRL1 of SEQ ID NO: 58, a CDRL2 of SEQ ID NO: 59, and a CDRL3 of SEQ ID NO: 60; (f) the VH comprises a CDRH1 of SEQ ID NO: 15, a CDRH2 of SEQ ID NO: 16, and a CDRH3 of SEQ ID NO: 17, and the VL comprises a CDRL1 of SEQ ID NO: 18, a CDRL2 of SEQ ID NO: 19, and a CDRL3 of SEQ ID NO: 20; (g) the VH comprises a CDRH1 of SEQ ID NO: 96, a CDRH2 of SEQ ID NO: 97, and a CDRH3 of SEQ ID NO: 98, and the VL comprises a CDRL1 of SEQ ID NO: 99, a CDRL2 of SEQ ID NO: 100, and a CDRL3 of SEQ ID NO: 101; (h) the VH comprises a CDRH1 of SEQ ID NO: 196, a CDRH2 of SEQ ID NO: 197, and a CDRH3 of SEQ ID NO: 198, and the VL comprises a CDRL1 of SEQ ID NO: 478, a CDRL2 of SEQ ID NO: 479, and a CDRL3 of SEQ ID NO: 480; (i) the VH comprises a CDRH1 of SEQ ID NO: 202, a CDRH2 of SEQ ID NO: 203, and a CDRH3 of SEQ ID NO: 204, and the VL comprises a CDRL1 of SEQ ID NO: 481, a CDRL2 of SEQ ID NO: 482, and a CDRL3 of SEQ ID NO: 483; (j) the VH comprises a CDRH1 of SEQ ID NO: 1170, a CDRH2 of SEQ ID NO: 1171, and a CDRH3 of SEQ ID NO: 1172, and the VL comprises a CDRL1 of SEQ ID NO: 1857, a CDRL2 of SEQ ID NO: 1858, and a CDRL3 of SEQ ID NO: 1859; (k) the VH comprises a CDRH1 of SEQ ID NO: 1173, a CDRH2 of SEQ ID NO: 1174, and a CDRH3 of SEQ ID NO: 1175, and the VL comprises a CDRL1 of SEQ ID NO: 1860, a CDRL2 of SEQ ID NO: 1861, and a CDRL3 of SEQ ID NO: 1862; (1) the VH comprises a CDRH1 of SEQ ID NO: 1176, a CDRH2 of SEQ ID NO: 1177, and a CDRH3 of SEQ ID NO: 1178, and the VL comprises a CDRL1 of SEQ ID NO: 1863, a CDRL2 of SEQ ID NO: 1864, and a CDRL3 of SEQ ID NO: 1865; (m) the VH comprises a CDRH1 of SEQ ID NO: 1179, a CDRH2 of SEQ ID NO: 1180, and a CDRH3 of SEQ ID NO: 1181, and the VL comprises a CDRL1 of SEQ ID NO: 1866, a CDRL2 of SEQ ID NO: 1867, and a CDRL3 of SEQ ID NO: 1868; (n) the VH comprises a CDRH1 of SEQ ID NO: 1182, a CDRH2 of SEQ ID NO: 1183, and a CDRH3 of SEQ ID NO: 1184, and the VL comprises a CDRL1 of SEQ ID NO: 1869, a CDRL2 of SEQ ID NO: 1870, and a CDRL3 of SEQ ID NO: 1871; (o) the VH comprises a CDRH1 of SEQ ID NO: 1185, a CDRH2 of SEQ ID NO: 1186, and a CDRH3 of SEQ ID NO: 1187, and the VL comprises a CDRL1 of SEQ ID NO: 1872, a CDRL2 of SEQ ID NO: 1873, and a CDRL3 of SEQ ID NO: 1874; (p) the VH comprises a CDRH1 of SEQ ID NO: 1188, a CDRH2 of SEQ ID NO: 1189, and a CDRH3 of SEQ ID NO: 1190, and the VL comprises a CDRL1 of SEQ ID NO: 1875, a CDRL2 of SEQ ID NO: 1876, and a CDRL3 of SEQ ID NO: 1877; (q) the VH comprises a CDRH1 of SEQ ID NO: 1524, a CDRH2 of SEQ ID NO: 1525, and a CDRH3 of SEQ ID NO: 1526, and the VL comprises a CDRL1 of SEQ ID NO: 2211, a CDRL2 of SEQ ID NO: 2212, and a CDRL3 of SEQ ID NO: 2213; (r) the VH comprises a CDRH1 of SEQ ID NO: 1527, a CDRH2 of SEQ ID NO: 1528, and a CDRH3 of SEQ ID NO: 1529, and the VL comprises a CDRL1 of SEQ ID NO: 2214, a CDRL2 of SEQ ID NO: 2215, and a CDRL3 of SEQ ID NO: 2216; (s) the VH comprises a CDRH1 of SEQ ID NO: 1530, a CDRH2 of SEQ ID NO: 1531, and a CDRH3 of SEQ ID NO: 1532, and the VL comprises a CDRL1 of SEQ ID NO: 2217, a CDRL2 of SEQ ID NO: 2218, and a CDRL3 of SEQ ID NO: 2219; (t) the VH comprises a CDRH1 of SEQ ID NO: 1533, a CDRH2 of SEQ ID NO: 1534, and a CDRH3 of SEQ ID NO: 1535, and the VL comprises a CDRL1 of SEQ ID NO: 2220, a CDRL2 of SEQ ID NO: 2221, and a CDRL3 of SEQ ID NO: 2222; (u) the VH comprises a CDRH1 of SEQ ID NO: 1539, a CDRH2 of SEQ ID NO: 1540, and a CDRH3 of SEQ ID NO: 1541, and the VL comprises a CDRL1 of SEQ ID NO: 2226, a CDRL2 of SEQ ID NO: 2227, and a CDRL3 of SEQ ID NO: 2228; (v) the VH comprises a CDRH1 of SEQ ID NO: 1542, a CDRH2 of SEQ ID NO: 1543, and a CDRH3 of SEQ ID NO: 1544, and the VL comprises a CDRL1 of SEQ ID NO: 2229, a CDRL2 of SEQ ID NO: 2230, and a CDRL3 of SEQ ID NO: 2231; (w) the VH comprises a CDRH1 of SEQ ID NO: 1548, a CDRH2 of SEQ ID NO: 1549, and a CDRH3 of SEQ ID NO: 1550, and the VL comprises a CDRL1 of SEQ ID NO: 2235, a CDRL2 of SEQ ID NO: 2236, and a CDRL3 of SEQ ID NO: 2237; (x) the VH comprises a CDRH1 of SEQ ID NO: 1551, a CDRH2 of SEQ ID NO: 1552, and a CDRH3 of SEQ ID NO: 1553, and the VL comprises a CDRL1 of SEQ ID NO: 2238, a CDRL2 of SEQ ID NO: 2239, and a CDRL3 of SEQ ID NO: 2240; (y) the VH comprises a CDRH1 of SEQ ID NO: 1554, a CDRH2 of SEQ ID NO: 1555, and a CDRH3 of SEQ ID NO: 1556, and the VL comprises a CDRL1 of SEQ ID NO: 2241, a CDRL2 of SEQ ID NO: 2242, and a CDRL3 of SEQ ID NO: 2243; (z) the VH comprises a CDRH1 of SEQ ID NO: 1557, a CDRH2 of SEQ ID NO: 1558, and a CDRH3 of SEQ ID NO: 1559, and the VL comprises a CDRL1 of SEQ ID NO: 2244, a CDRL2 of SEQ ID NO: 2245, and a CDRL3 of SEQ ID NO: 2246; (aa) the VH comprises a CDRH1 of SEQ ID NO: 1560, a CDRH2 of SEQ ID NO: 1561, and a CDRH3 of SEQ ID NO: 1562, and the VL comprises a CDRL1 of SEQ ID NO: 2247, a CDRL2 of SEQ ID NO: 2248, and a CDRL3 of SEQ ID NO: 2249; (bb) the VH comprises a CDRH1 of SEQ ID NO: 1563, a CDRH2 of SEQ ID NO: 1564, and a CDRH3 of SEQ ID NO: 1565, and the VL comprises a CDRL1 of SEQ ID NO: 2250, a CDRL2 of SEQ ID NO: 2251, and a CDRL3 of SEQ ID NO: 2252; (cc) the VH comprises a CDRH1 of SEQ ID NO: 1566, a CDRH2 of SEQ ID NO: 1567, and a CDRH3 of SEQ ID NO: 1568, and the VL comprises a CDRL1 of SEQ ID NO: 2253, a CDRL2 of SEQ ID NO: 2254, and a CDRL3 of SEQ ID NO: 2255; (dd) the VH comprises a CDRH1 of SEQ ID NO: 1572, a CDRH2 of SEQ ID NO: 1573, and a CDRH3 of SEQ ID NO: 1574, and the VL comprises a CDRL1 of SEQ ID NO: 2259, a CDRL2 of SEQ ID NO: 2260, and a CDRL3 of SEQ ID NO: 2261; (ee) the VH comprises a CDRH1 of SEQ ID NO: 1575, a CDRH2 of SEQ ID NO: 1576, and a CDRH3 of SEQ ID NO: 1577, and the VL comprises a CDRL1 of SEQ ID NO: 2262, a CDRL2 of SEQ ID NO: 2263, and a CDRL3 of SEQ ID NO: 2264; (ff) the VH comprises a CDRH1 of SEQ ID NO: 1578, a CDRH2 of SEQ ID NO: 1579, and a CDRH3 of SEQ ID NO: 1580, and the VL comprises a CDRL1 of SEQ ID NO: 2265, a CDRL2 of SEQ ID NO: 2266, and a CDRL3 of SEQ ID NO: 2267; (gg) the VH comprises a CDRH1 of SEQ ID NO: 1587, a CDRH2 of SEQ ID NO: 1588, and a CDRH3 of SEQ ID NO: 1589, and the VL comprises a CDRL1 of SEQ ID NO: 2274, a CDRL2 of SEQ ID NO: 2275, and a CDRL3 of SEQ ID NO: 2276; (hh) the VH comprises a CDRH1 of SEQ ID NO: 1590, a CDRH2 of SEQ ID NO: 1591, and a CDRH3 of SEQ ID NO: 1592, and the VL comprises a CDRL1 of SEQ ID NO: 2277, a CDRL2 of SEQ ID NO: 2278, and a CDRL3 of SEQ ID NO: 2279; (ii) the VH comprises a CDRH1 of SEQ ID NO: 1593, a CDRH2 of SEQ ID NO: 1594, and a CDRH3 of SEQ ID NO: 1595, and the VL comprises a CDRL1 of SEQ ID NO: 2280, a CDRL2 of SEQ ID NO: 2281, and a CDRL3 of SEQ ID NO: 2282; (jj) the VH comprises a CDRH1 of SEQ ID NO: 1596, a CDRH2 of SEQ ID NO: 1597, and a CDRH3 of SEQ ID NO: 1598, and the VL comprises a CDRL1 of SEQ ID NO: 2283, a CDRL2 of SEQ ID NO: 2284, and a CDRL3 of SEQ ID NO: 2285; (kk) the VH comprises a CDRH1 of SEQ ID NO: 1599, a CDRH2 of SEQ ID NO: 1560, and a CDRH3 of SEQ ID NO: 1561, and the VL comprises a CDRL1 of SEQ ID NO: 2286, a CDRL2 of SEQ ID NO: 2287, and a CDRL3 of SEQ ID NO: 2288; (11) the VH comprises a CDRH1 of SEQ ID NO: 1602, a CDRH2 of SEQ ID NO: 1603, and a CDRH3 of SEQ ID NO: 1604, and the VL comprises a CDRL1 of SEQ ID NO: 2289, a CDRL2 of SEQ ID NO: 2290, and a CDRL3 of SEQ ID NO: 2291; (mm) the VH comprises a CDRH1 of SEQ ID NO: 1605, a CDRH2 of SEQ ID NO: 1606, and a CDRH3 of SEQ ID NO: 1607, and the VL comprises a CDRL1 of SEQ ID NO: 2292, a CDRL2 of SEQ ID NO: 2293, and a CDRL3 of SEQ ID NO: 2294; (nn) the VH comprises a CDRH1 of SEQ ID NO: 1608, a CDRH2 of SEQ ID NO: 1609, and a CDRH3 of SEQ ID NO: 1610, and the VL comprises a CDRL1 of SEQ ID NO: 2295, a CDRL2 of SEQ ID NO: 2296, and a CDRL3 of SEQ ID NO: 2297; (oo) the VH comprises a CDRH1 of SEQ ID NO: 1611, a CDRH2 of SEQ ID NO: 1612, and a CDRH3 of SEQ ID NO: 1613, and the VL comprises a CDRL1 of SEQ ID NO: 2298, a CDRL2 of SEQ ID NO: 2299, and a CDRL3 of SEQ ID NO: 2300; (pp) the VH comprises a CDRH1 of SEQ ID NO: 1614, a CDRH2 of SEQ ID NO: 1615, and a CDRH3 of SEQ ID NO: 1616, and the VL comprises a CDRL1 of SEQ ID NO: 2301, a CDRL2 of SEQ ID NO: 2302, and a CDRL3 of SEQ ID NO: 2303; (qq) the VH comprises a CDRH1 of SEQ ID NO: 1617, a CDRH2 of SEQ ID NO: 1618, and a CDRH3 of SEQ ID NO: 1619, and the VL comprises a CDRL1 of SEQ ID NO: 2304, a CDRL2 of SEQ ID NO: 2305, and a CDRL3 of SEQ ID NO: 2306; (rr) the VH comprises a CDRH1 of SEQ ID NO: 1626, a CDRH2 of SEQ ID NO: 1627, and a CDRH3 of SEQ ID NO: 1628, and the VL comprises a CDRL1 of SEQ ID NO: 2313, a CDRL2 of SEQ ID NO: 2314, and a CDRL3 of SEQ ID NO: 2315; (ss) the VH comprises a CDRH1 of SEQ ID NO: 1629, a CDRH2 of SEQ ID NO: 1630, and a CDRH3 of SEQ ID NO: 1631, and the VL comprises a CDRL1 of SEQ ID NO: 2316, a CDRL2 of SEQ ID NO: 2317, and a CDRL3 of SEQ ID NO: 2318; (tt) the VH comprises a CDRH1 of SEQ ID NO: 1632, a CDRH2 of SEQ ID NO: 1633, and a CDRH3 of SEQ ID NO: 1634, and the VL comprises a CDRL1 of SEQ ID NO: 2319, a CDRL2 of SEQ ID NO: 2320, and a CDRL3 of SEQ ID NO: 2321; (uu) the VH comprises a CDRH1 of SEQ ID NO: 1635, a CDRH2 of SEQ ID NO: 1636, and a CDRH3 of SEQ ID NO: 1637, and the VL comprises a CDRL1 of SEQ ID NO: 2322, a CDRL2 of SEQ ID NO: 2323, and a CDRL3 of SEQ ID NO: 2324; (vv) the VH comprises a CDRH1 of SEQ ID NO: 1638, a CDRH2 of SEQ ID NO: 1639, and a CDRH3 of SEQ ID NO: 1640, and the VL comprises a CDRL1 of SEQ ID NO: 2325, a CDRL2 of SEQ ID NO: 2326, and a CDRL3 of SEQ ID NO: 2327; (ww) the VH comprises a CDRH1 of SEQ ID NO: 1641, a CDRH2 of SEQ ID NO: 1642, and a CDRH3 of SEQ ID NO: 1643, and the VL comprises a CDRL1 of SEQ ID NO: 2328, a CDRL2 of SEQ ID NO: 2329, and a CDRL3 of SEQ ID NO: 2330; (xx) the VH comprises a CDRH1 of SEQ ID NO: 1644, a CDRH2 of SEQ ID NO: 1645, and a CDRH3 of SEQ ID NO: 1646, and the VL comprises a CDRL1 of SEQ ID NO: 2331, a CDRL2 of SEQ ID NO: 2332, and a CDRL3 of SEQ ID NO: 2333; (yy) the VH comprises a CDRH1 of SEQ ID NO: 1647, a CDRH2 of SEQ ID NO: 1648, and a CDRH3 of SEQ ID NO: 1649, and the VL comprises a CDRL1 of SEQ ID NO: 2334, a CDRL2 of SEQ ID NO: 2335, and a CDRL3 of SEQ ID NO: 2336; (zz) the VH comprises a CDRH1 of SEQ ID NO: 1650, a CDRH2 of SEQ ID NO: 1651, and a CDRH3 of SEQ ID NO: 1652, and the VL comprises a CDRL1 of SEQ ID NO: 2337, a CDRL2 of SEQ ID NO: 2338, and a CDRL3 of SEQ ID NO: 2339; (aaa) the VH comprises a CDRH1 of SEQ ID NO: 1653, a CDRH2 of SEQ ID NO: 1654, and a CDRH3 of SEQ ID NO: 1655, and the VL comprises a CDRL1 of SEQ ID NO: 2340, a CDRL2 of SEQ ID NO: 2341, and a CDRL3 of SEQ ID NO: 2342; (bbb) the VH comprises a CDRH1 of SEQ ID NO: 1656, a CDRH2 of SEQ ID NO: 1657, and a CDRH3 of SEQ ID NO: 1658, and the VL comprises a CDRL1 of SEQ ID NO: 2343, a CDRL2 of SEQ ID NO: 2344, and a CDRL3 of SEQ ID NO: 2345; or (ccc) the VH comprises a CDRH1 of SEQ ID NO: 1659, a CDRH2 of SEQ ID NO: 1660, and a CDRH3 of SEQ ID NO: 1661, and the VL comprises a CDRL1 of SEQ ID NO: 2346, a CDRL2 of SEQ ID NO: 2347, and a CDRL3 of SEQ ID NO: 2348.

In some embodiments, the second antigen recognition domain comprises a scFv comprising a VH and a VL, wherein: (a) the VH comprises SEQ ID NO: 82 and the VL comprises SEQ ID NO: 84; (b) the VH comprises SEQ ID NO: 21 and the VL comprises SEQ ID NO: 23; (c) the VH comprises SEQ ID NO: 31 and the VL comprises SEQ ID NO: 33; (d) the VH comprises SEQ ID NO: 41 and the VL comprises SEQ ID NO: 43; (e) the VH comprises SEQ ID NO: 51 and the VL comprises SEQ ID NO: 53; (f) the VH comprises SEQ ID NO: 61 and the VL comprises SEQ ID NO: 63; (g) the VH comprises SEQ ID NO: 693 and the VL comprises SEQ ID NO: 66; (h) the VH comprises SEQ ID NO: 694 and the VL comprises SEQ ID NO: 69; (i) the VH comprises SEQ ID NO: 695 and the VL comprises SEQ ID NO: 72; (j) the VH comprises SEQ ID NO: 74 and the VL comprises SEQ ID NO: 76; (k) the VH comprises SEQ ID NO: 78 and the VL comprises SEQ ID NO: 80; (1) the VH comprises SEQ ID NO: 11 and the VL comprises SEQ ID NO: 13; (m) the VH comprises SEQ ID NO: 92 and the VL comprises SEQ ID NO: 94; (n) the VH comprises SEQ ID NO: 102 and the VL comprises SEQ ID NO: 103; (o) the VH comprises SEQ ID NO: 104 and the VL comprises SEQ ID NO: 105; (p) the VH comprises SEQ ID NO: 712 and the VL comprises SEQ ID NO: 713; (q) the VH comprises SEQ ID NO: 714 and the VL comprises SEQ ID NO: 715; (r) the VH comprises SEQ ID NO: 716 and the VL comprises SEQ ID NO: 717; (s) the VH comprises SEQ ID NO: 718 and the VL comprises SEQ ID NO: 719; (t) the VH comprises SEQ ID NO: 720 and the VL comprises SEQ ID NO: 721; (u) the VH comprises SEQ ID NO: 722 and the VL comprises SEQ ID NO: 723; (v) the VH comprises SEQ ID NO: 724 and the VL comprises SEQ ID NO: 725; (w) the VH comprises SEQ ID NO: 948 and the VL comprises SEQ ID NO: 949; (x) the VH comprises SEQ ID NO: 950 and the VL comprises SEQ ID NO: 951; (y) the VH comprises SEQ ID NO: 952 and the VL comprises SEQ ID NO: 953; (z) the VH comprises SEQ ID NO: 954 and the VL comprises SEQ ID NO: 955; (aa) the VH comprises SEQ ID NO: 958 and the VL comprises SEQ ID NO: 959; (bb) the VH comprises SEQ ID NO: 960 and the VL comprises SEQ ID NO: 961; (cc) the VH comprises SEQ ID NO: 964 and the VL comprises SEQ ID NO: 965; (dd) the VH comprises SEQ ID NO: 966 and the VL comprises SEQ ID NO: 967; (ee) the VH comprises SEQ ID NO: 968 and the VL comprises SEQ ID NO: 969; (ff) the VH comprises SEQ ID NO: 970 and the VL comprises SEQ ID NO: 971; (gg) the VH comprises SEQ ID NO: 972 and the VL comprises SEQ ID NO: 973; (hh) the VH comprises SEQ ID NO: 974 and the VL comprises SEQ ID NO: 975; (ii) the VH comprises SEQ ID NO: 976 and the VL comprises SEQ ID NO: 977; (jj) the VH comprises SEQ ID NO: 980 and the VL comprises SEQ ID NO: 981; (kk) the VH comprises SEQ ID NO: 982 and the VL comprises SEQ ID NO: 983; (11) the VH comprises SEQ ID NO: 984 and the VL comprises SEQ ID NO: 985; (mm) the VH comprises SEQ ID NO: 990 and the VL comprises SEQ ID NO: 991; (nn) the VH comprises SEQ ID NO: 992 and the VL comprises SEQ ID NO: 993; (oo) the VH comprises SEQ ID NO: 994 and the VL comprises SEQ ID NO: 995; (pp) the VH comprises SEQ ID NO: 996 and the VL comprises SEQ ID NO: 997; (qq) the VH comprises SEQ ID NO: 998 and the VL comprises SEQ ID NO: 999; (rr) the VH comprises SEQ ID NO: 1000 and the VL comprises SEQ ID NO: 1001; (ss) the VH comprises SEQ ID NO: 1002 and the VL comprises SEQ ID NO: 1003; (tt) the VH comprises SEQ ID NO: 1004 and the VL comprises SEQ ID NO: 1005; (uu) the VH comprises SEQ ID NO: 1006 and the VL comprises SEQ ID NO: 1007; (vv) the VH comprises SEQ ID NO: 1008 and the VL comprises SEQ ID NO: 1009; (ww) the VH comprises SEQ ID NO: 1010 and the VL comprises SEQ ID NO: 1011; (xx) the VH comprises SEQ ID NO: 1016 and the VL comprises SEQ ID NO: 1017; (yy) the VH comprises SEQ ID NO: 1018 and the VL comprises SEQ ID NO: 1019; (zz) the VH comprises SEQ ID NO: 1020 and the VL comprises SEQ ID NO: 1021; (aaa) the VH comprises SEQ ID NO: 1022 and the VL comprises SEQ ID NO: 1023; (bbb) the VH comprises SEQ ID NO: 1024 and the VL comprises SEQ ID NO: 1025; (ccc) the VH comprises SEQ ID NO: 1026 and the VL comprises SEQ ID NO: 1027; (ddd) the VH comprises SEQ ID NO: 1028 and the VL comprises SEQ ID NO: 1029; (eee) the VH comprises SEQ ID NO: 1030 and the VL comprises SEQ ID NO: 1031; (fff) the VH comprises SEQ ID NO: 1032 and the VL comprises SEQ ID NO: 1033; (ggg) the VH comprises SEQ ID NO: 1034 and the VL comprises SEQ ID NO: 1035; (hhh) the VH comprises SEQ ID NO: 1036 and the VL comprises SEQ ID NO: 1037; or (iii) the VH comprises SEQ ID NO: 1038 and the VL comprises SEQ ID NO: 1039.

In some embodiments, the second antigen recognition domain comprises a single domain antibody fragment, an adnectin peptide, an affibody, an afflilin, an affimer, an affitin, an alphabody, an anticalin, an avimer, a DARPin (Designed Ankyrin Repeat Protein), a Fynomer, a Kunitz domain peptide, a monobody, a centyrin, an aptamer, a T cell receptor (TCR)-like antibody, a single chain TCR (scTCR), or a portion of any of the foregoing.

In some embodiments, the second antigen recognition domain comprises a human CD27 extracellular domain.

In some embodiments, the extracellular domain comprises a hinge.

In some embodiments, the transmembrane domain comprises a CD8, CD16, CD27, CD28, 2B4, NKG2D, NKp44, NKp46, NKp30, NKp80, DNAM-1, CD3 zeta, CD3 epsilon, CD3 gamma, CD3 delta, CD45, CD4, CD5, CD9, CD22, CD33, CD37, CD64, CD80, CD86, CD134, CD137, CD154, ICOS/CD278, GITR/CD357, DAP10, DAP12 or erythropoietin receptor transmembrane domain, a portion of any of the foregoing, or a combination of any of the foregoing.

In some embodiments, the intracellular domain comprises one or more costimulatory domain(s). In certain embodiments, the one or more costimulatory domain(s) are selected from the group consisting of: a CD28 costimulatory domain, a 4-1BB costimulatory domain, a DAP10 costimulatory domain, a DAP12 costimulatory domain, a 2B4 costimulatory domain, a OX40 costimulatory domain, an OX40L costimulatory domain, a ICOS costimulatory domain, or a CD27 costimulatory domain, or a portion of any of the foregoing.

In some embodiments, the intracellular domain comprises an activation domain. In certain embodiments, the activation domain comprises a DAP12, FCER1G, FCGR2A, or CD3zeta activation domain, or a portion of any of the foregoing.

In some embodiments, the aforementioned method further comprises: (e) contacting the population of NK cells with at least one polynucleotide encoding at least one exogenous polypeptide.

In some embodiments, the at least one exogenous polypeptide comprises a cytokine, a chemokine, a ligand, a receptor, a monoclonal antibody, a bispecific T cell engager, a peptide, or an enzyme, a subunit or a portion of the foregoing, or any combination of the foregoing. In certain embodiments, the at least one exogenous polypeptide comprises a cytokine. In particular, the cytokine comprises IL-15, membrane-bound IL-15 (mbIL-15), IL-2, membrane-bound IL-2, IL-12, membrane-bound IL-12, IL-18, membrane-bound IL-18, IL-21, membrane-bound IL-21, p40, LIGHT, CD40L, FLT3L, 4-1BBL, or FASL.

In some embodiments, the at least one exogenous polypeptide comprises IL-15RA, IL-15, or is a fusion protein comprising IL-15 and IL-15RA. In other embodiments, the at least one exogenous polypeptide is a tethered IL-21, a tethered IL-12, or a tethered IL-18. In some embodiments, the at least one exogenous polypeptide comprises a first exogenous polypeptide comprising mbIL-15 and a second exogenous polypeptide comprising IL-15RA. Alternatively, the at least one exogenous polypeptide comprises a receptor selected from the group consisting of: CSF-1R, a CXC chemokine receptor, a CC chemokine receptor, a CX3C chemokine receptor, a XC chemokine receptor, or a chemokine-binding fragment thereof. In some embodiments, the at least one exogenous polypeptide is a protein that overcomes immunosuppression of the tumor microenvironment. In certain embodiments, the protein comprises a TGFbeta signal converter. In particular, the TGFbeta signal converter comprises a TGFbeta receptor extracellular domain and an NK cell intracellular domain. In certain embodiments, the protein comprises a TGFbeta decoy receptor comprising a TGFbeta receptor extracellular domain and optionally, a transmembrane domain. In particular, the transmembrane domain is a transmembrane domain from a protein that is not a TGFbeta receptor. Alternatively, the transmembrane domain is a transmembrane domain from the TGFbeta receptor.

In some embodiments, the at least one exogenous polypeptide comprises a CAR comprising at least one antigen recognition domain that specifically binds an antigen other than human CD70. In certain embodiments, the antigen other than human CD70 is selected from the group consisting of: CAIX, CD19, CD20, CD22, CD33, CD37, CD79a, CD79b, CD96, CD123, CD138, CLL-1, CXCR5, BCMA, FOLR2, FCRL5, FLT3, GPRC5D, HAVCR1, Her2, mesothelin, MUC16, EGFR, EGFRVIII, IL13Ra2, Trop2, GPC3, FOLR1, and GD2. In some embodiments, the at least one exogenous polypeptide comprises a safety switch protein.

In some embodiments, the aforementioned method further comprises linking at least one exogenous polypeptide to at least one NK cell of the NK cell population by chemical conjugation or using a sortase enzyme.

Further provided herein is a genetically engineered natural killer (NK) cell modified to have: a) a decreased level of total expressed CD70 polypeptide compared to the level of total expressed CD70 polypeptide in a wild-type NK cell, and/or b) a decreased level of surface expressed CD70 polypeptide compared to the level of surface expressed CD70 in a wild-type NK cell.

In some embodiments, the genetically engineered NK cell comprises a disrupted CD70 gene. In certain embodiments, the genetically engineered NK cell comprises a knockout or knockdown of a CD70 gene. In some embodiments, the genetically engineered NK cell comprises at least about 30% less of surface expressed CD70 polypeptide and/or total expressed CD70 polypeptide than the wild-type NK cell. In some embodiments, the level of CD70 mRNA in the genetically engineered NK cell is reduced as compared to the level of CD70 mRNA in a wild-type NK cell.

In some embodiments, the genetically engineered NK cell comprises a siRNA that targets CD70 mRNA, a nucleic acid encoding a siRNA that targets CD70 mRNA, a shRNA that targets CD70 mRNA, a nucleic acid encoding a shRNA that targets CD70 mRNA, a nucleic acid encoding a tandem shRNA that targets CD70 mRNA, a tandem shRNA that targets CD70 mRNA, a nucleic acid encoding a ribozyme that targets CD70 mRNA, or a ribozyme that targets CD70 mRNA, or a combination of any of the foregoing. In some embodiments, the genetically engineered NK cell comprises an RNA guided endonuclease and a gRNA targeting a CD70 gene. In some embodiments, the genetically engineered NK cell comprises a PEBL or a nucleic acid encoding a PEBL, wherein the PEBL comprises a first antigen recognition domain that specifically binds human CD70 and one or more of a localizing domain, an intracellular retention domain and an ER retention domain.

In some embodiments, the genetically engineered NK cell is derived from umbilical cord blood cells, PBMCs, mobilized unstimulated leukapheresis products (PBSCs), unmobilized PBSCs, human embryonic stem cells (hESCs), induced pluripotent stem cells (iPSCs), mesenchymal stem cells (MSCs), hematopoietic stem cells (HSCs), bone marrow or CD34⁺ cells.

In some embodiments, the genetically engineered NK cell is a human NK cell.

In some embodiments, the genetically engineered NK cell comprises a CAR and/or a polynucleotide encoding the CAR, wherein the CAR comprises (a) an extracellular domain comprising a second antigen recognition domain that specifically binds human CD70; (b) a transmembrane domain; and (c) an intracellular domain. In certain embodiments, the second antigen recognition domain comprises a scFv comprising a VH and a VL, wherein (a) the VH comprises a CDRH1 of SEQ ID NO: 86, a CDRH2 of SEQ ID NO: 87, and a CDRH3 of SEQ ID NO: 88, and the VL comprises a CDRL1 of SEQ ID NO: 89, a CDRL2 of SEQ ID NO: 90, and a CDRL3 of SEQ ID NO: 91; (b) the VH comprises a CDRH1 of SEQ ID NO: 25, a CDRH2 of SEQ ID NO: 26, and a CDRH3 of SEQ ID NO: 27, and the VL comprises a CDRL1 of SEQ ID NO: 28, a CDRL2 of SEQ ID NO: 29, and a CDRL3 of SEQ ID NO: 30; (c) the VH comprises a CDRH1 of SEQ ID NO: 35, a CDRH2 of SEQ ID NO: 36, and a CDRH3 of SEQ ID NO: 37, and the VL comprises a CDRL1 of SEQ ID NO: 38, a CDRL2 of SEQ ID NO: 39, and a CDRL3 of SEQ ID NO: 40; (d) the VH comprises a CDRH1 of SEQ ID NO: 45, a CDRH2 of SEQ ID NO: 46, and a CDRH3 of SEQ ID NO: 47, and the VL comprises a CDRL1 of SEQ ID NO: 48, a CDRL2 of SEQ ID NO: 49, and a CDRL3 of SEQ ID NO: 50; (e) the VH comprises a CDRH1 of SEQ ID NO: 55, a CDRH2 of SEQ ID NO: 56, and a CDRH3 of SEQ ID NO: 57, and the VL comprises a CDRL1 of SEQ ID NO: 58, a CDRL2 of SEQ ID NO: 59, and a CDRL3 of SEQ ID NO: 60; (f) the VH comprises a CDRH1 of SEQ ID NO: 15, a CDRH2 of SEQ ID NO: 16, and a CDRH3 of SEQ ID NO: 17, and the VL comprises a CDRL1 of SEQ ID NO: 18, a CDRL2 of SEQ ID NO: 19, and a CDRL3 of SEQ ID NO: 20; (g) the VH comprises a CDRH1 of SEQ ID NO: 96, a CDRH2 of SEQ ID NO: 97, and a CDRH3 of SEQ ID NO: 98, and the VL comprises a CDRL1 of SEQ ID NO: 99, a CDRL2 of SEQ ID NO: 100, and a CDRL3 of SEQ ID NO: 101; (h) the VH comprises a CDRH1 of SEQ ID NO: 196, a CDRH2 of SEQ ID NO: 197, and a CDRH3 of SEQ ID NO: 198, and the VL comprises a CDRL1 of SEQ ID NO: 478, a CDRL2 of SEQ ID NO: 479, and a CDRL3 of SEQ ID NO: 480; (i) the VH comprises a CDRH1 of SEQ ID NO: 202, a CDRH2 of SEQ ID NO: 203, and a CDRH3 of SEQ ID NO: 204, and the VL comprises a CDRL1 of SEQ ID NO: 481, a CDRL2 of SEQ ID NO: 482, and a CDRL3 of SEQ ID NO: 483; (j) the VH comprises a CDRH1 of SEQ ID NO: 1170, a CDRH2 of SEQ ID NO: 1171, and a CDRH3 of SEQ ID NO: 1172, and the VL comprises a CDRL1 of SEQ ID NO: 1857, a CDRL2 of SEQ ID NO: 1858, and a CDRL3 of SEQ ID NO: 1859; (k) the VH comprises a CDRH1 of SEQ ID NO: 1173, a CDRH2 of SEQ ID NO: 1174, and a CDRH3 of SEQ ID NO: 1175, and the VL comprises a CDRL1 of SEQ ID NO: 1860, a CDRL2 of SEQ ID NO: 1861, and a CDRL3 of SEQ ID NO: 1862; (1) the VH comprises a CDRH1 of SEQ ID NO: 1176, a CDRH2 of SEQ ID NO: 1177, and a CDRH3 of SEQ ID NO: 1178, and the VL comprises a CDRL1 of SEQ ID NO: 1863, a CDRL2 of SEQ ID NO: 1864, and a CDRL3 of SEQ ID NO: 1865; (m) the VH comprises a CDRH1 of SEQ ID NO: 1179, a CDRH2 of SEQ ID NO: 1180, and a CDRH3 of SEQ ID NO: 1181, and the VL comprises a CDRL1 of SEQ ID NO: 1866, a CDRL2 of SEQ ID NO: 1867, and a CDRL3 of SEQ ID NO: 1868; (n) the VH comprises a CDRH1 of SEQ ID NO: 1182, a CDRH2 of SEQ ID NO: 1183, and a CDRH3 of SEQ ID NO: 1184, and the VL comprises a CDRL1 of SEQ ID NO: 1869, a CDRL2 of SEQ ID NO: 1870, and a CDRL3 of SEQ ID NO: 1871; (o) the VH comprises a CDRH1 of SEQ ID NO: 1185, a CDRH2 of SEQ ID NO: 1186, and a CDRH3 of SEQ ID NO: 1187, and the VL comprises a CDRL1 of SEQ ID NO: 1872, a CDRL2 of SEQ ID NO: 1873, and a CDRL3 of SEQ ID NO: 1874; (p) the VH comprises a CDRH1 of SEQ ID NO: 1188, a CDRH2 of SEQ ID NO: 1189, and a CDRH3 of SEQ ID NO: 1190, and the VL comprises a CDRL1 of SEQ ID NO: 1875, a CDRL2 of SEQ ID NO: 1876, and a CDRL3 of SEQ ID NO: 1877; (q) the VH comprises a CDRH1 of SEQ ID NO: 1524, a CDRH2 of SEQ ID NO: 1525, and a CDRH3 of SEQ ID NO: 1526, and the VL comprises a CDRL1 of SEQ ID NO: 2211, a CDRL2 of SEQ ID NO: 2212, and a CDRL3 of SEQ ID NO: 2213; (r) the VH comprises a CDRH1 of SEQ ID NO: 1527, a CDRH2 of SEQ ID NO: 1528, and a CDRH3 of SEQ ID NO: 1529, and the VL comprises a CDRL1 of SEQ ID NO: 2214, a CDRL2 of SEQ ID NO: 2215, and a CDRL3 of SEQ ID NO: 2216; (s) the VH comprises a CDRH1 of SEQ ID NO: 1530, a CDRH2 of SEQ ID NO: 1531, and a CDRH3 of SEQ ID NO: 1532, and the VL comprises a CDRL1 of SEQ ID NO: 2217, a CDRL2 of SEQ ID NO: 2218, and a CDRL3 of SEQ ID NO: 2219; (t) the VH comprises a CDRH1 of SEQ ID NO: 1533, a CDRH2 of SEQ ID NO: 1534, and a CDRH3 of SEQ ID NO: 1535, and the VL comprises a CDRL1 of SEQ ID NO: 2220, a CDRL2 of SEQ ID NO: 2221, and a CDRL3 of SEQ ID NO: 2222; (u) the VH comprises a CDRH1 of SEQ ID NO: 1539, a CDRH2 of SEQ ID NO: 1540, and a CDRH3 of SEQ ID NO: 1541, and the VL comprises a CDRL1 of SEQ ID NO: 2226, a CDRL2 of SEQ ID NO: 2227, and a CDRL3 of SEQ ID NO: 2228; (v) the VH comprises a CDRH1 of SEQ ID NO: 1542, a CDRH2 of SEQ ID NO: 1543, and a CDRH3 of SEQ ID NO: 1544, and the VL comprises a CDRL1 of SEQ ID NO: 2229, a CDRL2 of SEQ ID NO: 2230, and a CDRL3 of SEQ ID NO: 2231; (w) the VH comprises a CDRH1 of SEQ ID NO: 1548, a CDRH2 of SEQ ID NO: 1549, and a CDRH3 of SEQ ID NO: 1550, and the VL comprises a CDRL1 of SEQ ID NO: 2235, a CDRL2 of SEQ ID NO: 2236, and a CDRL3 of SEQ ID NO: 2237; (x) the VH comprises a CDRH1 of SEQ ID NO: 1551, a CDRH2 of SEQ ID NO: 1552, and a CDRH3 of SEQ ID NO: 1553, and the VL comprises a CDRL1 of SEQ ID NO: 2238, a CDRL2 of SEQ ID NO: 2239, and a CDRL3 of SEQ ID NO: 2240; (y) the VH comprises a CDRH1 of SEQ ID NO: 1554, a CDRH2 of SEQ ID NO: 1555, and a CDRH3 of SEQ ID NO: 1556, and the VL comprises a CDRL1 of SEQ ID NO: 2241, a CDRL2 of SEQ ID NO: 2242, and a CDRL3 of SEQ ID NO: 2243; (z) the VH comprises a CDRH1 of SEQ ID NO: 1557, a CDRH2 of SEQ ID NO: 1558, and a CDRH3 of SEQ ID NO: 1559, and the VL comprises a CDRL1 of SEQ ID NO: 2244, a CDRL2 of SEQ ID NO: 2245, and a CDRL3 of SEQ ID NO: 2246; (aa) the VH comprises a CDRH1 of SEQ ID NO: 1560, a CDRH2 of SEQ ID NO: 1561, and a CDRH3 of SEQ ID NO: 1562, and the VL comprises a CDRL1 of SEQ ID NO: 2247, a CDRL2 of SEQ ID NO: 2248, and a CDRL3 of SEQ ID NO: 2249; (bb) the VH comprises a CDRH1 of SEQ ID NO: 1563, a CDRH2 of SEQ ID NO: 1564, and a CDRH3 of SEQ ID NO: 1565, and the VL comprises a CDRL1 of SEQ ID NO: 2250, a CDRL2 of SEQ ID NO: 2251, and a CDRL3 of SEQ ID NO: 2252; (cc) the VH comprises a CDRH1 of SEQ ID NO: 1566, a CDRH2 of SEQ ID NO: 1567, and a CDRH3 of SEQ ID NO: 1568, and the VL comprises a CDRL1 of SEQ ID NO: 2253, a CDRL2 of SEQ ID NO: 2254, and a CDRL3 of SEQ ID NO: 2255; (dd) the VH comprises a CDRH1 of SEQ ID NO: 1572, a CDRH2 of SEQ ID NO: 1573, and a CDRH3 of SEQ ID NO: 1574, and the VL comprises a CDRL1 of SEQ ID NO: 2259, a CDRL2 of SEQ ID NO: 2260, and a CDRL3 of SEQ ID NO: 2261; (ee) the VH comprises a CDRH1 of SEQ ID NO: 1575, a CDRH2 of SEQ ID NO: 1576, and a CDRH3 of SEQ ID NO: 1577, and the VL comprises a CDRL1 of SEQ ID NO: 2262, a CDRL2 of SEQ ID NO: 2263, and a CDRL3 of SEQ ID NO: 2264; (ff) the VH comprises a CDRH1 of SEQ ID NO: 1578, a CDRH2 of SEQ ID NO: 1579, and a CDRH3 of SEQ ID NO: 1580, and the VL comprises a CDRL1 of SEQ ID NO: 2265, a CDRL2 of SEQ ID NO: 2266, and a CDRL3 of SEQ ID NO: 2267; (gg) the VH comprises a CDRH1 of SEQ ID NO: 1587, a CDRH2 of SEQ ID NO: 1588, and a CDRH3 of SEQ ID NO: 1589, and the VL comprises a CDRL1 of SEQ ID NO: 2274, a CDRL2 of SEQ ID NO: 2275, and a CDRL3 of SEQ ID NO: 2276; (hh) the VH comprises a CDRH1 of SEQ ID NO: 1590, a CDRH2 of SEQ ID NO: 1591, and a CDRH3 of SEQ ID NO: 1592, and the VL comprises a CDRL1 of SEQ ID NO: 2277, a CDRL2 of SEQ ID NO: 2278, and a CDRL3 of SEQ ID NO: 2279; (ii) the VH comprises a CDRH1 of SEQ ID NO: 1593, a CDRH2 of SEQ ID NO: 1594, and a CDRH3 of SEQ ID NO: 1595, and the VL comprises a CDRL1 of SEQ ID NO: 2280, a CDRL2 of SEQ ID NO: 2281, and a CDRL3 of SEQ ID NO: 2282; (jj) the VH comprises a CDRH1 of SEQ ID NO: 1596, a CDRH2 of SEQ ID NO: 1597, and a CDRH3 of SEQ ID NO: 1598, and the VL comprises a CDRL1 of SEQ ID NO: 2283, a CDRL2 of SEQ ID NO: 2284, and a CDRL3 of SEQ ID NO: 2285; (kk) the VH comprises a CDRH1 of SEQ ID NO: 1599, a CDRH2 of SEQ ID NO: 1560, and a CDRH3 of SEQ ID NO: 1561, and the VL comprises a CDRL1 of SEQ ID NO: 2286, a CDRL2 of SEQ ID NO: 2287, and a CDRL3 of SEQ ID NO: 2288; (11) the VH comprises a CDRH1 of SEQ ID NO: 1602, a CDRH2 of SEQ ID NO: 1603, and a CDRH3 of SEQ ID NO: 1604, and the VL comprises a CDRL1 of SEQ ID NO: 2289, a CDRL2 of SEQ ID NO: 2290, and a CDRL3 of SEQ ID NO: 2291; (mm) the VH comprises a CDRH1 of SEQ ID NO: 1605, a CDRH2 of SEQ ID NO: 1606, and a CDRH3 of SEQ ID NO: 1607, and the VL comprises a CDRL1 of SEQ ID NO: 2292, a CDRL2 of SEQ ID NO: 2293, and a CDRL3 of SEQ ID NO: 2294; (nn) the VH comprises a CDRH1 of SEQ ID NO: 1608, a CDRH2 of SEQ ID NO: 1609, and a CDRH3 of SEQ ID NO: 1610, and the VL comprises a CDRL1 of SEQ ID NO: 2295, a CDRL2 of SEQ ID NO: 2296, and a CDRL3 of SEQ ID NO: 2297; (oo) the VH comprises a CDRH1 of SEQ ID NO: 1611, a CDRH2 of SEQ ID NO: 1612, and a CDRH3 of SEQ ID NO: 1613, and the VL comprises a CDRL1 of SEQ ID NO: 2298, a CDRL2 of SEQ ID NO: 2299, and a CDRL3 of SEQ ID NO: 2300; (pp) the VH comprises a CDRH1 of SEQ ID NO: 1614, a CDRH2 of SEQ ID NO: 1615, and a CDRH3 of SEQ ID NO: 1616, and the VL comprises a CDRL1 of SEQ ID NO: 2301, a CDRL2 of SEQ ID NO: 2302, and a CDRL3 of SEQ ID NO: 2303; (qq) the VH comprises a CDRH1 of SEQ ID NO: 1617, a CDRH2 of SEQ ID NO: 1618, and a CDRH3 of SEQ ID NO: 1619, and the VL comprises a CDRL1 of SEQ ID NO: 2304, a CDRL2 of SEQ ID NO: 2305, and a CDRL3 of SEQ ID NO: 2306; (rr) the VH comprises a CDRH1 of SEQ ID NO: 1626, a CDRH2 of SEQ ID NO: 1627, and a CDRH3 of SEQ ID NO: 1628, and the VL comprises a CDRL1 of SEQ ID NO: 2313, a CDRL2 of SEQ ID NO: 2314, and a CDRL3 of SEQ ID NO: 2315; (ss) the VH comprises a CDRH1 of SEQ ID NO: 1629, a CDRH2 of SEQ ID NO: 1630, and a CDRH3 of SEQ ID NO: 1631, and the VL comprises a CDRL1 of SEQ ID NO: 2316, a CDRL2 of SEQ ID NO: 2317, and a CDRL3 of SEQ ID NO: 2318; (tt) the VH comprises a CDRH1 of SEQ ID NO: 1632, a CDRH2 of SEQ ID NO: 1633, and a CDRH3 of SEQ ID NO: 1634, and the VL comprises a CDRL1 of SEQ ID NO: 2319, a CDRL2 of SEQ ID NO: 2320, and a CDRL3 of SEQ ID NO: 2321; (uu) the VH comprises a CDRH1 of SEQ ID NO: 1635, a CDRH2 of SEQ ID NO: 1636, and a CDRH3 of SEQ ID NO: 1637, and the VL comprises a CDRL1 of SEQ ID NO: 2322, a CDRL2 of SEQ ID NO: 2323, and a CDRL3 of SEQ ID NO: 2324; (vv) the VH comprises a CDRH1 of SEQ ID NO: 1638, a CDRH2 of SEQ ID NO: 1639, and a CDRH3 of SEQ ID NO: 1640, and the VL comprises a CDRL1 of SEQ ID NO: 2325, a CDRL2 of SEQ ID NO: 2326, and a CDRL3 of SEQ ID NO: 2327; (ww) the VH comprises a CDRH1 of SEQ ID NO: 1641, a CDRH2 of SEQ ID NO: 1642, and a CDRH3 of SEQ ID NO: 1643, and the VL comprises a CDRL1 of SEQ ID NO: 2328, a CDRL2 of SEQ ID NO: 2329, and a CDRL3 of SEQ ID NO: 2330; (xx) the VH comprises a CDRH1 of SEQ ID NO: 1644, a CDRH2 of SEQ ID NO: 1645, and a CDRH3 of SEQ ID NO: 1646, and the VL comprises a CDRL1 of SEQ ID NO: 2331, a CDRL2 of SEQ ID NO: 2332, and a CDRL3 of SEQ ID NO: 2333; (yy) the VH comprises a CDRH1 of SEQ ID NO: 1647, a CDRH2 of SEQ ID NO: 1648, and a CDRH3 of SEQ ID NO: 1649, and the VL comprises a CDRL1 of SEQ ID NO: 2334, a CDRL2 of SEQ ID NO: 2335, and a CDRL3 of SEQ ID NO: 2336; (zz) the VH comprises a CDRH1 of SEQ ID NO: 1650, a CDRH2 of SEQ ID NO: 1651, and a CDRH3 of SEQ ID NO: 1652, and the VL comprises a CDRL1 of SEQ ID NO: 2337, a CDRL2 of SEQ ID NO: 2338, and a CDRL3 of SEQ ID NO: 2339; (aaa) the VH comprises a CDRH1 of SEQ ID NO: 1653, a CDRH2 of SEQ ID NO: 1654, and a CDRH3 of SEQ ID NO: 1655, and the VL comprises a CDRL1 of SEQ ID NO: 2340, a CDRL2 of SEQ ID NO: 2341, and a CDRL3 of SEQ ID NO: 2342; (bbb) the VH comprises a CDRH1 of SEQ ID NO: 1656, a CDRH2 of SEQ ID NO: 1657, and a CDRH3 of SEQ ID NO: 1658, and the VL comprises a CDRL1 of SEQ ID NO: 2343, a CDRL2 of SEQ ID NO: 2344, and a CDRL3 of SEQ ID NO: 2345; or (ccc) the VH comprises a CDRH1 of SEQ ID NO: 1659, a CDRH2 of SEQ ID NO: 1660, and a CDRH3 of SEQ ID NO: 1661, and the VL comprises a CDRL1 of SEQ ID NO: 2346, a CDRL2 of SEQ ID NO: 2347, and a CDRL3 of SEQ ID NO: 2348.

In some embodiments of the aforementioned genetically engineered NK cell, the second antigen recognition domain comprises a scFv comprising a VH and a VL, wherein (a) the VH comprises SEQ ID NO: 82 and the VL comprises SEQ ID NO: 84; (b) the VH comprises SEQ ID NO: 21 and the VL comprises SEQ ID NO: 23; (c) the VH comprises SEQ ID NO: 31 and the VL comprises SEQ ID NO: 33; (d) the VH comprises SEQ ID NO: 41 and the VL comprises SEQ ID NO: 43; (e) the VH comprises SEQ ID NO: 51 and the VL comprises SEQ ID NO: 53; (f) the VH comprises SEQ ID NO: 61 and the VL comprises SEQ ID NO: 63; (g) the VH comprises SEQ ID NO: 693 and the VL comprises SEQ ID NO: 66; (h) the VH comprises SEQ ID NO: 694 and the VL comprises SEQ ID NO: 69; (i) the VH comprises SEQ ID NO: 695 and the VL comprises SEQ ID NO: 72; (j) the VH comprises SEQ ID NO: 74 and the VL comprises SEQ ID NO: 76; (k) the VH comprises SEQ ID NO: 78 and the VL comprises SEQ ID NO: 80; (1) the VH comprises SEQ ID NO: 11 and the VL comprises SEQ ID NO: 13; (m) the VH comprises SEQ ID NO: 92 and the VL comprises SEQ ID NO: 94; (n) the VH comprises SEQ ID NO: 102 and the VL comprises SEQ ID NO: 103; (o) the VH comprises SEQ ID NO: 104 and the VL comprises SEQ ID NO: 105; (p) the VH comprises SEQ ID NO: 712 and the VL comprises SEQ ID NO: 713; (q) the VH comprises SEQ ID NO: 714 and the VL comprises SEQ ID NO: 715; (r) the VH comprises SEQ ID NO: 716 and the VL comprises SEQ ID NO: 717; (s) the VH comprises SEQ ID NO: 718 and the VL comprises SEQ ID NO: 719; (t) the VH comprises SEQ ID NO: 720 and the VL comprises SEQ ID NO: 721; (u) the VH comprises SEQ ID NO: 722 and the VL comprises SEQ ID NO: 723; (v) the VH comprises SEQ ID NO: 724 and the VL comprises SEQ ID NO: 725; (w) the VH comprises SEQ ID NO: 948 and the VL comprises SEQ ID NO: 949; (x) the VH comprises SEQ ID NO: 950 and the VL comprises SEQ ID NO: 951; (y) the VH comprises SEQ ID NO: 952 and the VL comprises SEQ ID NO: 953; (z) the VH comprises SEQ ID NO: 954 and the VL comprises SEQ ID NO: 955; (aa) the VH comprises SEQ ID NO: 958 and the VL comprises SEQ ID NO: 959; (bb) the VH comprises SEQ ID NO: 960 and the VL comprises SEQ ID NO: 961; (cc) the VH comprises SEQ ID NO: 964 and the VL comprises SEQ ID NO: 965; (dd) the VH comprises SEQ ID NO: 966 and the VL comprises SEQ ID NO: 967; (ee) the VH comprises SEQ ID NO: 968 and the VL comprises SEQ ID NO: 969; (ff) the VH comprises SEQ ID NO: 970 and the VL comprises SEQ ID NO: 971; (gg) the VH comprises SEQ ID NO: 972 and the VL comprises SEQ ID NO: 973; (hh) the VH comprises SEQ ID NO: 974 and the VL comprises SEQ ID NO: 975; (ii) the VH comprises SEQ ID NO: 976 and the VL comprises SEQ ID NO: 977; (jj) the VH comprises SEQ ID NO: 980 and the VL comprises SEQ ID NO: 981; (kk) the VH comprises SEQ ID NO: 982 and the VL comprises SEQ ID NO: 983; (11) the VH comprises SEQ ID NO: 984 and the VL comprises SEQ ID NO: 985; (mm) the VH comprises SEQ ID NO: 990 and the VL comprises SEQ ID NO: 991; (nn) the VH comprises SEQ ID NO: 992 and the VL comprises SEQ ID NO: 993; (oo) the VH comprises SEQ ID NO: 994 and the VL comprises SEQ ID NO: 995; (pp) the VH comprises SEQ ID NO: 996 and the VL comprises SEQ ID NO: 997; (qq) the VH comprises SEQ ID NO: 998 and the VL comprises SEQ ID NO: 999; (rr) the VH comprises SEQ ID NO: 1000 and the VL comprises SEQ ID NO: 1001; (ss) the VH comprises SEQ ID NO: 1002 and the VL comprises SEQ ID NO: 1003; (tt) the VH comprises SEQ ID NO: 1004 and the VL comprises SEQ ID NO: 1005; (uu) the VH comprises SEQ ID NO: 1006 and the VL comprises SEQ ID NO: 1007; (vv) the VH comprises SEQ ID NO: 1008 and the VL comprises SEQ ID NO: 1009; (ww) the VH comprises SEQ ID NO: 1010 and the VL comprises SEQ ID NO: 1011; (xx) the VH comprises SEQ ID NO: 1016 and the VL comprises SEQ ID NO: 1017; (yy) the VH comprises SEQ ID NO: 1018 and the VL comprises SEQ ID NO: 1019; (zz) the VH comprises SEQ ID NO: 1020 and the VL comprises SEQ ID NO: 1021; (aaa) the VH comprises SEQ ID NO: 1022 and the VL comprises SEQ ID NO: 1023; (bbb) the VH comprises SEQ ID NO: 1024 and the VL comprises SEQ ID NO: 1025; (ccc) the VH comprises SEQ ID NO: 1026 and the VL comprises SEQ ID NO: 1027; (ddd) the VH comprises SEQ ID NO: 1028 and the VL comprises SEQ ID NO: 1029; (eee) the VH comprises SEQ ID NO: 1030 and the VL comprises SEQ ID NO: 1031; (fff) the VH comprises SEQ ID NO: 1032 and the VL comprises SEQ ID NO: 1033; (ggg) the VH comprises SEQ ID NO: 1034 and the VL comprises SEQ ID NO: 1035; (hhh) the VH comprises SEQ ID NO: 1036 and the VL comprises SEQ ID NO: 1037; or (iii) the VH comprises SEQ ID NO: 1038 and the VL comprises SEQ ID NO: 1039.

In some embodiments, the second antigen recognition domain comprises a single domain antibody fragment, an adnectin peptide, an affibody, an afflilin, an affimer, an affitin, an alphabody, an anticalin, an avimer, a DARPin (Designed Ankyrin Repeat Protein), a Fynomer, a Kunitz domain peptide, a monobody, a centyrin, an aptamer, a T cell receptor (TCR)-like antibody, a single chain TCR (scTCR), or a portion of any of the foregoing.

In some embodiments, the second antigen recognition domain comprises a human CD27 extracellular domain. In some embodiments, the extracellular domain comprises a hinge.

In some embodiments of the aforementioned genetically engineered NK cell, the transmembrane domain comprises a CD8, CD16, CD27, CD28, NKG2D, NKp44, NKp46, NKp30, NKp80, DNAM-1, CD3 zeta, CD3 epsilon, CD3 gamma, CD3 delta, CD45, CD4, CD5, CD9, CD22, CD33, CD37, CD64, CD80, CD86, CD134, CD137, CD154, ICOS/CD278, GITR/CD357, DAP10, DAP12 or erythropoietin receptor transmembrane domain, a portion of any of the foregoing, or a combination of any of the foregoing.

In some embodiments, the intracellular domain comprises one or more costimulatory domain(s). In certain embodiments, the one or more costimulatory domain(s) are selected from the group consisting of: a CD28 costimulatory domain, a 4-1BB costimulatory domain, a DAP10 costimulatory domain, a DAP12 costimulatory domain, a 2B4 costimulatory domain, a OX40 costimulatory domain, an OX40L costimulatory domain, a ICOS costimulatory domain, or a CD27 costimulatory domain, or a portion of any of the foregoing.

In some embodiments, the intracellular domain comprises an activation domain. In certain embodiments, the activation domain comprises a DAP12, FCER1G, FCGR2A, or CD3zeta intracellular signaling domain, or a portion of any of the foregoing.

In some embodiments, the genetically engineered NK cell comprises a CAR and/or a polynucleotide encoding the CAR, wherein the CAR comprises an amino acid an amino acid sequence that is at least 90%, at least 91%, at least 92%, at least 93%, at least 94%, at least 95%, at least 96%, at least 97%, at least 98%, at least 99% or at least 100% identical to the amino acid sequence of SEQ ID NO: 637, 639, 641, 643, 645, 647, 700, 2561-2593, 2697-2736 or 2737-2882.

In some embodiments, the aforementioned genetically engineered NK cell further comprises at least one exogenous polypeptide. In certain embodiments, the at least one exogenous polypeptide comprises a cytokine, chemokine, ligand, receptor, monoclonal antibody, bispecific T cell engager, peptide or enzyme, a subunit or a portion of the foregoing, or any combination of the foregoing. In particular, the at least one exogenous polypeptide comprises a cytokine, wherein the cytokine comprises IL-15, membrane-bound IL-15 (mbIL-15), IL-2, membrane-bound IL-2, IL-12, membrane-bound IL-12, IL-18, membrane-bound IL-18, IL-21, membrane-bound IL-21, p40, LIGHT, CD40L, FLT3L, 4-1BBL, or FASL. In some embodiments, the at least one exogenous polypeptide comprises IL-15RA, IL-15, or is a fusion protein comprising IL-15 and IL-15RA. In some embodiments, the at least one exogenous polypeptide is a tethered IL-21, a tethered IL-12, or a tethered IL-18.

In some embodiments, the genetically engineered NK cell further comprises a first exogenous polypeptide comprising mbIL-15 and a second exogenous polypeptide comprising IL-15RA.

In some embodiments, the at least one exogenous polypeptide comprises a receptor selected from the group consisting of: CSF-1R, a CXC chemokine receptor, a CC chemokine receptor, a CX3C chemokine receptor, a XC chemokine receptor, or a chemokine-binding fragment thereof.

In some embodiments, the at least one exogenous polypeptide is a protein that overcomes immunosuppression of the tumor microenvironment. In certain embodiments, the protein comprises a TGFbeta signal converter. In particular, the TGFbeta signal converter comprises a TGFbeta receptor extracellular domain and an NK cell intracellular domain. In other embodiments, the protein comprises a TGFbeta decoy receptor comprising a TGFbeta receptor extracellular domain and optionally, a transmembrane domain. In certain embodiments, the transmembrane domain is a transmembrane domain from a protein that is not a TGFbeta receptor. Alternatively, the transmembrane domain is a transmembrane domain from the TGFbeta receptor.

In some embodiments, the at least one exogenous polypeptide comprises a CAR comprising at least one antigen recognition domain that specifically binds an antigen other than human CD70. In certain embodiments, the antigen other than human CD70 is selected from the group consisting of: CAIX, CD19, CD20, CD22, CD33, CD37, CD79a, CD79b, CD96, CD123, CD138, CLL-1, CXCR5, BCMA, FOLR2, FCRL5, FLT3, GPRC5D, HAVCR1, Her2, mesothelin, MUC16, EGFR, EGFRVIII, IL13Ra2, Trop2, GPC3, FOLR1, or GD2. In some embodiments, the at least one exogenous polypeptide comprises a safety switch protein.

In some embodiments, the genetically engineered NK cell comprises at least one exogenous polypeptide linked to the genetically engineered NK cell by chemical conjugation or by a sortase-mediated transpeptidation reaction.

In some embodiments, the genetically engineered NK has a reduced likelihood of fratricide by a NK cell expressing an anti-CD70 CAR compared to the likelihood of fratricide of a wild-type NK cell.

In some embodiments, the genetically engineered NK cell exhibits greater fold cell expansion than a wildtype NK cell.

Further provided herein is a population of cells, wherein at least about 30% of cells in the population are the genetically engineered NK cell described hereinabove.

Also provided herein is a pharmaceutical composition comprising the aforementioned genetically engineered NK cell or the aforementioned population of cells, and a pharmaceutically acceptable carrier, diluent, or excipient.

Further provided herein is a method for treating a cancer in a subject by administering to the subject an effective amount of the aforementioned population of cells or the aforementioned pharmaceutical composition.

In some embodiments, the cancer is a CD70-positive cancer. In certain embodiments, the cancer is a solid tumor. In particular, the cancer is selected from the group consisting of: renal cancer (e.g., renal clear cell carcinoma, renal non-clear cell carcinoma), lung cancer, pleural mesothelioma, colorectal cancer, ovarian cancer, breast cancer, head and neck cancer (e.g., head and neck squamous cell carcinoma), esophageal squamous cell carcinoma, melanoma, pancreatic cancer, gastric cancer, cervical cancer (e.g., cervical squamous cell carcinoma), esophageal cancer, lung cancer, sarcoma, seminoma, non-seminomatous germ cell tumor, and glioblastoma. In other embodiments, the cancer is a hematologic malignancy. In particular, the hematologic malignancy is acute myeloid leukemia (AML), non-Hodgkin's lymphoma (e.g., diffuse large B cell lymphoma (DLBCL), mantle cell lymphoma (MCL)), acute lymphoblastic leukemia, peripheral T cell lymphoma (PTCL), anaplastic large cell lymphoma (ALCL), myelodysplastic syndrome (MDS), multiple myeloma, Waldenstrom's macroglobulinemia, mature B cell neoplasms, or chronic lymphocytic leukemia (CLL).

In some embodiments, the method for treating a cancer further comprises administering an additional therapeutic agent.

### BRIEF DESCRIPTION OF THE DRAWINGS

FIG. 1 is a schematic diagram of exemplary chimeric antigen receptors of the disclosure that specifically bind to CD70. Signal Seq represents signal peptide sequence. Signal Seq represents signal peptide sequence. TM represents transmembrane sequence. Costim 1 and Costim 2 represent costimulatory domain sequences. Signaling represents activation domain sequences.

FIG. 2 is a schematic diagram of exemplary constructs of the disclosure that encode a membrane bound IL-12 polypeptide.

FIG. 3 is a schematic diagram of exemplary constructs of the disclosure that encode a soluble or secreted IL-15 and/or IL15Ra.

FIGS. 4A-FIG. 4D are schematic diagrams of exemplary constructs of the disclosure that encode a CAR and an shRNA. FIG. 4A shows a MND promoter or EF1a promoter regulated CAR located upstream of a U6 promoter regulated shRNA. Transcription may occur through the MND/EF1a promoter and the U6 promoters in the same direction or in the opposite direction. FIG. 4B shows a MND promoter or EF1a promoter regulated CAR located downstream of a U6 promoter regulated shRNA. Transcription may occur through the MND/EF1a promoter and the U6 promoters in the same direction or in the opposite direction. FIG. 4C shows a MND promoter or EF1a promoter regulated CAR and cytokine element(s), located upstream of a U6 promoter regulated shRNA. Transcription may occur through the MND/EF1a promoter and the U6 promoters in the same direction or in the opposite direction. FIG. 4D shows a MND promoter or EF1a promoter regulated CAR and cytokine element(s), located downstream of a U6 promoter regulated shRNA. Transcription may occur through the MND/EF1a promoter and the U6 promoters in the same direction or in the opposite direction.

FIG. 5 depicts a wild type TcBuster transposase amino acid sequence, highlighting amino acids that may be points of contact with DNA. Large bold lettering indicates catalytic triad amino acids; lettering with boxes indicates amino acids that when substituted to a positive charged amino acid increases transposition; italicized and lowercased lettering indicates positive charged amino acids that when substituted to a different amino acid decreases transposition; bold italicized and underlined indicates amino acids that when substituted to a positive charged amino acid increases transposition, and when substituted to a negative charged amino acid decreases transposition; underlined lettering indicates amino acids that could be positive charged amino acids based on protein sequence alignment to the Buster subfamily.

FIG. 6 is a series of histograms depicting that CD70 expression increases upon activation of peripheral blood NK cells with K562-4-1BBL-mbIL-21 Feeder Cells.

FIG. 7 is a series of flow cytometry scatterplots showing that CD70 is efficiently knocked out from peripheral blood NK cells.

FIG. 8A-FIG. 8C is a series of graphs showing transduction and expansion of CAR-NK. FIG. 8A shows that the percentage of live NK cells expressing the CD70-targeting CARs Construct #1 (an exemplary CD27 CAR of SEQ ID NO: 643) and Construct #2 (an exemplary ScFv specific for CD70 of SEQ ID NO: 2565), or GFP are similar in CD70WT NK and CD70 KO NK cells. FIG. 8B shows that cell counts of CD70 wild-type (WT) NK engineered to express CD70 targeting Construct #1 or Construct #2 CARs were significantly lower than those of CD70 KO NK cells expressing CD70 targeting Construct #1 or Construct #2 CARs. As a control, NK cells engineered to express a GFP had similar cell counts in CD70 WT and CD70 NK cells. FIG. 8C shows the viability of CD70 WT NK engineered to express the CD70 targeting CARs, Construct #1 or Construct #2 CARs were less than 25% viable while viability remained above 80% in CD70 KO NK cells engineered to express CD70 targeting CARs, Construct #1 and Construct #2. CD70 WT NK cells engineered to express a GFP control were 58% viable, while CD70 KO NK cells engineered to express a GFP control were 90 percent viable.

FIG. 9A and FIG. 9B is a series of graphs showing CD70 CAR mediated fratricide of autologous CD70 wild-type NK cells. FIG. 9A shows #CTV+ cells/#target cells only for autologous CTV+CD70 WT NK cells at various E/T ratios (4:1, 2:1, 1:1, or 0.5:1). FIG. 9B shows #CTV+ cells/#target cells only for autologous CTV+CD70 KO NK cells at various E/T ratios (4:1, 2:1, 1:1, or 0.5:1).

FIG. 10 shows CD70 CAR mediated killing of MOLM-13 cell line. CD70 KO NK cells engineered to express CD70 targeting CARs, Construct #1 and Construct #2, demonstrate specific killing of MOLM-13 target cells expressing WT CD70, but do not demonstrate specific killing of MOLM-13 CD70 KO target cells.

FIG. 11 is a graph showing that anti-CD70 CAR transduction and CD70 expression were inversely correlated. Peripheral blood natural killer (PBNK) cells were transduced with increasing concentrations of virus to express an anti-CD70 CAR comprising a CD27 extracellular domain (“anti-CD70 CAR (CD27 receptor)”) and the percentage of CAR-positive cells (circles) and CD70-positive cells (squares) four days post-transduction is shown. As control, PBNKs were transduced with increasing concentrations of virus to express ZsGreen fluorescent protein (“ZsGreen”) and the percentage of CD70-positive cells (triangles) at four days post-transduction is shown.

### DETAILED DESCRIPTION

The present disclosure overcomes problems associated with current technologies by providing NK cells and antigen-specific NK cells for immunotherapy, such as for the treatment of immune-related diseases, including cancer and autoimmune disorders, as well as infection including but not limited to viruses, such as CMV, EBV, and HIV. The present disclosure is based, at least in part, on the discovery that while CD70 is not expressed in resting peripheral blood NK cells, the protein is upregulated in response to NK cell activation. The upregulation of CD70 following activation is detrimental to the culture of NK cells genetically modified to express chimeric antigen receptors (CARs) that specifically bind to CD70 as it may result in fratricide. Accordingly, the present disclosure provides fratricide-resistant NK cells and methods of generating the cells by, e.g., contacting the cells with at least one CD70 inhibitor. Such cells can efficiently target and kill cells expressing CD70 without incurring significant NK cell fratricide during culture. In some embodiments, the NK cells disclosed herein may comprise reduced levels of CD70 (e.g., protein and/or mRNA) and/or exhibit reduced CD70 activity. In some embodiments, this reduction of CD70 levels and/or CD70 activity is achieved by contacting NK cells with at least one CD70 inhibitor. In addition, the present disclosure is also based, at least in part, on the discovery that contacting an NK cell or a population of NK cells with a CD70 inhibitor results in enhanced expansion capability as compared to an NK cell or a population of NK cells that has not been contacted with a CD70 inhibitor. Increasing cell expansion is desirable to improve the production of NK cells for therapeutic applications. Accordingly, methods of making populations of NK cells are also provided.

The methods described herein can result in an increase in the expansion (e.g., fold-expansion) of an NK cell or population of NK cells (e.g., about a 1-fold to about 500-fold, about a 1-told to about a 450-fold, about a 1-fold to about a 400-fold, about a 1-fold to about a 350-fold, about a 1-fold to about a 300-fold, about a 1-fold to about a 250-fold, about a 1-fold to about a 200-fold, about a 1-fold to about a 180-fold, about a 1-fold to about a 160-fold, about a 1-fold to about a 140-fold, about a 1-fold to about a 120-fold, about a 1-fold to about a 100-fold, about a 1-fold to about a 80-fold, about a 1-fold to about a 60-fold, about a 1-fold to about a 50-fold, about a 1-fold to about a 40-fold, about a 1-fold to about a 30-fold, about 1-fold to about a 25-fold, about a 1-fold- to about a 20-fold, about a 1-fold to about a 15-fold, about a 1-fold to about to about a 10-fold, about a 1-fold to about a 5-fold, about a 5-fold to about 500-fold, about a 5-told to about a 450-fold, about a 5-fold to about a 400-fold, about a 5-fold to about a 350-fold, about a 5-fold to about a 300-fold, about a 5-fold to about a 250-fold, about a 5-fold to about a 200-fold, about a 5-fold to about a 180-fold, about a 5-fold to about a 160-fold, about a 5-fold to about a 140-fold, about a 5-fold to about a 120-fold, about a 5-fold to about a 100-fold, about a 5-fold to about a 80-fold, about a 5-fold to about a 60-fold, about a 5-fold to about a 50-fold, about a 5-fold to about a 40-fold, about a 5-fold to about a 30-fold, about 5-fold to about a 25-fold, about a 5-fold- to about a 20-fold, about a 5-fold to about a 15-fold, about a 5-fold to about to about a 10-fold, about a 10-fold to about 500-fold, about a 10-told to about a 450-fold, about a 10-fold to about a 400-fold, about a 10-fold to about a 350-fold, about a 10-fold to about a 300-fold, about a 10-fold to about a 250-fold, about a 10-fold to about a 200-fold, about a 10-fold to about a 180-fold, about a 10-fold to about a 160-fold, about a 10-fold to about a 140-fold, about a 10-fold to about a 120-fold, about a 10-fold to about a 100-fold, about a 10-fold to about a 80-fold, about a 10-fold to about a 60-fold, about a 10-fold to about a 50-fold, about a 10-fold to about a 40-fold, about a 10-fold to about a 30-fold, about 10-fold to about a 25-fold, about a 10-fold- to about a 20-fold, about a 10-fold to about a 15-fold, about a 15-fold to about 500-fold, about a 15-told to about a 450-fold, about a 15-fold to about a 400-fold, about a 15-fold to about a 350-fold, about a 15-fold to about a 300-fold, about a 15-fold to about a 250-fold, about a 15-fold to about a 200-fold, about a 15-fold to about a 180-fold, about a 15-fold to about a 160-fold, about a 15-fold to about a 140-fold, about a 15-fold to about a 120-fold, about a 15-fold to about a 100-fold, about a 15-fold to about a 80-fold, about a 15-fold to about a 60-fold, about a 15-fold to about a 50-fold, about a 15-fold to about a 40-fold, about a 15-fold to about a 30-fold, about 15-fold to about a 25-fold, about a 15-fold- to about a 20-fold, about a 20-fold to about 500-fold, about a 20-told to about a 450-fold, about a 20-fold to about a 400-fold, about a 20-fold to about a 350-fold, about a 20-fold to about a 300-fold, about a 20-fold to about a 250-fold, about a 20-fold to about a 200-fold, about a 20-fold to about a 180-fold, about a 20-fold to about a 160-fold, about a 20-fold to about a 140-fold, about a 20-fold to about a 120-fold, about a 20-fold to about a 100-fold, about a 20-fold to about a 80-fold, about a 20-fold to about a 60-fold, about a 20-fold to about a 50-fold, about a 20-fold to about a 40-fold, about a 20-fold to about a 30-fold, about 20-fold to about a 25-fold, about a 25-fold to about 500-fold, about a 25-told to about a 450-fold, about a 25-fold to about a 400-fold, about a 25-fold to about a 350-fold, about a 25-fold to about a 300-fold, about a 25-fold to about a 250-fold, about a 25-fold to about a 200-fold, about a 25-fold to about a 180-fold, about a 25-fold to about a 160-fold, about a 25-fold to about a 140-fold, about a 25-fold to about a 120-fold, about a 25-fold to about a 100-fold, about a 25-fold to about a 80-fold, about a 25-fold to about a 60-fold, about a 25-fold to about a 50-fold, about a 25-fold to about a 40-fold, about a 25-fold to about a 30-fold, about a 30-fold to about 500-fold, about a 30-told to about a 450-fold, about a 30-fold to about a 400-fold, about a 30-fold to about a 350-fold, about a 30-fold to about a 300-fold, about a 30-fold to about a 250-fold, about a 30-fold to about a 200-fold, about a 30-fold to about a 180-fold, about a 30-fold to about a 160-fold, about a 30-fold to about a 140-fold, about a 30-fold to about a 120-fold, about a 30-fold to about a 100-fold, about a 30-fold to about a 80-fold, about a 30-fold to about a 60-fold, about a 30-fold to about a 50-fold, about a 30-fold to about a 40-fold, about a 40-fold to about 500-fold, about a 40-told to about a 450-fold, about a 40-fold to about a 400-fold, about a 40-fold to about a 350-fold, about a 40-fold to about a 300-fold, about a 40-fold to about a 250-fold, about a 40-fold to about a 200-fold, about a 40-fold to about a 180-fold, about a 40-fold to about a 160-fold, about a 40-fold to about a 140-fold, about a 40-fold to about a 120-fold, about a 40-fold to about a 100-fold, about a 40-fold to about a 80-fold, about a 40-fold to about a 60-fold, about a 40-fold to about a 50-fold, about a 50-fold to about 500-fold, about a 50-told to about a 450-fold, about a 50-fold to about a 400-fold, about a 50-fold to about a 350-fold, about a 50-fold to about a 300-fold, about a 50-fold to about a 250-fold, about a 50-fold to about a 200-fold, about a 50-fold to about a 180-fold, about a 50-fold to about a 160-fold, about a 50-fold to about a 140-fold, about a 50-fold to about a 120-fold, about a 50-fold to about a 100-fold, about a 50-fold to about a 80-fold, about a 50-fold to about a 60-fold, about a 60-fold to about 500-fold, about a 60-told to about a 450-fold, about a 60-fold to about a 400-fold, about a 60-fold to about a 350-fold, about a 60-fold to about a 300-fold, about a 60-fold to about a 250-fold, about a 60-fold to about a 200-fold, about a 60-fold to about a 180-fold, about a 60-fold to about a 160-fold, about a 60-fold to about a 140-fold, about a 60-fold to about a 120-fold, about a 60-fold to about a 100-fold, about a 60-fold to about a 80-fold, about a 80-fold to about 500-fold, about a 80-told to about a 450-fold, about a 80-fold to about a 400-fold, about a 80-fold to about a 350-fold, about a 80-fold to about a 300-fold, about a 80-fold to about a 250-fold, about a 80-fold to about a 200-fold, about a 80-fold to about a 180-fold, about a 80-fold to about a 160-fold, about a 80-fold to about a 140-fold, about a 80-fold to about a 120-fold, about a 80-fold to about a 100-fold, about a 100-fold to about 500-fold, about a 100-told to about a 450-fold, about a 100-fold to about a 400-fold, about a 100-fold to about a 350-fold, about a 100-fold to about a 300-fold, about a 100-fold to about a 250-fold, about a 100-fold to about a 200-fold, about a 100-fold to about a 180-fold, about a 100-fold to about a 160-fold, about a 100-fold to about a 140-fold, about a 100-fold to about a 120-fold, about a 120-fold to about 500-fold, about a 120-told to about a 450-fold, about a 120-fold to about a 400-fold, about a 120-fold to about a 350-fold, about a 120-fold to about a 300-fold, about a 120-fold to about a 250-fold, about a 120-fold to about a 200-fold, about a 120-fold to about a 180-fold, about a 120-fold to about a 160-fold, about a 120-fold to about a 140-fold, about a 140-fold to about 500-fold, about a 140-told to about a 450-fold, about a 140-fold to about a 400-fold, about a 140-fold to about a 350-fold, about a 140-fold to about a 300-fold, about a 140-fold to about a 250-fold, about a 140-fold to about a 200-fold, about a 140-fold to about a 180-fold, about a 140-fold to about a 160-fold, about a 160-fold to about 500-fold, about a 160-told to about a 450-fold, about a 160-fold to about a 400-fold, about a 160-fold to about a 350-fold, about a 160-fold to about a 300-fold, about a 160-fold to about a 250-fold, about a 160-fold to about a 200-fold, about a 160-fold to about a 180-fold, about a 180-fold to about 500-fold, about a 180-told to about a 450-fold, about a 180-fold to about a 400-fold, about a 180-fold to about a 350-fold, about a 180-fold to about a 300-fold, about a 180-fold to about a 250-fold, about a 180-fold to about a 200-fold, about a 200-fold to about 500-fold, about a 200-told to about a 450-fold, about a 200-fold to about a 400-fold, about a 200-fold to about a 350-fold, about a 200-fold to about a 300-fold, about a 200-fold to about a 250-fold, about a 250-fold to about 500-fold, about a 250-told to about a 450-fold, about a 250-fold to about a 400-fold, about a 250-fold to about a 350-fold, about a 250-fold to about a 300-fold, about a 300-fold to about 500-fold, about a 300-told to about a 450-fold, about a 300-fold to about a 400-fold, about a 300-fold to about a 350-fold, about a 350-fold to about 500-fold, about a 350-told to about a 450-fold, about a 350-fold to about a 400-fold, about a 400-fold to about 500-fold, about a 400-told to about a 450-fold, or about a 450-fold to about a 500-fold, expansion) as compared to a NK cell or a population of NK cells that is not contacted with the CD70 inhibitor (e.g., a wild-type NK cell or a population of wild-type NK cells).

In some embodiments, the present disclosure provides NK cells which express one or more chimeric antigen receptors (CARs) that specifically recognize CD70. To enhance signaling, the CAR may be linked to an activation domain. To generate a more potent receptor that functions optimally in NK cells, the receptor may have a costimulatory domain (including but not limited to CD28, 4-1BB, DAP12, DAP10, 2B4, OX40, OX40L, CD27, ICOS or any combination of thereof), as well as a CD3ζ, FCGR2A or FCER1G activation domain. Thus, the present disclosure also provides methods for application of NK cell immunotherapy to target CD70 derived from tumors and pathogens. Further, unlike T cells, NK cells from an allogeneic source carry a lower risk of inducing graft-versus-host disease; thus, the use of allogeneic NK cells with CARs provide a potential source of CAR-engineered NK cells for adoptive therapy.

Moreover, the present disclosure further provides immune cells, such as NK cells, comprising one or more exogenous polypeptides in addition to the CAR. For example, the cells may comprise at least two antigen receptors, such as a combination of two CARs, for dual targeting of tumors. To allow for the enhanced in vivo persistence of NK cells, the cells may be engineered to express an exogenous polypeptide comprising IL-15, IL-15 and IL-15 receptor alpha (IL-15RA or IL-15Ra) complex or another cytokine such as IL-2, IL-12, IL-21, IL-18, TNFalpha, IFNbeta, LIGHT, CD40L, FLT3L, HVEM, LTa, LTb, VEGFc, or a combination thereof. In some embodiments, the exogenous polypeptide comprises a membrane-bound IL-15, a tethered IL-21, a tethered IL-12, or a tethered IL-18. In some embodiments, the cells may be engineered to express an exogenous polypeptide comprising soluble or secreted IL-15. In some embodiments, the additional exogenous polypeptide comprises IL-15RA or a fusion protein comprising IL-15 and IL-15RA. In some embodiments, the NK cell comprises a first additional exogenous polypeptide and a second additional exogenous polypeptide. In some embodiments, (a) the first additional exogenous polypeptide comprises mbIL-15 and the second additional exogenous polypeptide comprises IL-15RA; or (b) the first additional exogenous polypeptide comprises soluble IL-15 and the second additional exogenous polypeptide comprises IL-15RA.

To allow for the NK cells to have enhanced ability to overcome the tumor microenvironment in vivo, the NK cells provided herein may be engineered to express a functional effector element such as a TGFβ signal converter, a TGFβ decoy receptor (e.g., a TGFβ dominant negative receptor) or a chemokine receptor. For example, a TGFβ signal converter may comprise a TGFβ receptor extracellular domain with the intracellular domain replaced with an NK cell intracellular domain, thereby converting a negative suppression signal into a NK cell stimulation signal. For example, a TGFβ decoy receptor may comprise a truncated TGFβ receptor that lacks the intracellualar signalling domain, thereby interfering with endogenous TGFβ receptor signalling and preventing TGFβ inhibition of the NK cells. In some embodiments, the TGFβ decoy receptor comprises the extracellular domain of a TGFβ receptor (e.g., the extracellular domain of TGFBR1 or TGFBR2) and the transmembrane domain of a TGFβ receptor (e.g., the transmembrane domain of TGFBR1 or TGFBR2). In some embodiments, a TGFβ decoy receptor comprises the extracellular domain of a TGFβ receptor (e.g., the extracellular domain of TGFBR1 or TGFBR2) and a heterologous transmembrane domain (e.g., any of the transmembrane domains provided herein (e.g., a CD28 transmembrane domain)). For example, the chemokine receptor may be CXCR4. Thus, the genetically engineered NK cells may express one or more CARs that bind to any combination of target antigens and may further express IL-15/IL-15RA complex or other cytokines, a TGFβ signal converter or a chemokine receptor. The NK cells may be derived from several sources including peripheral blood, cord blood, bone marrow, stem cells, induced pluripotent stem cells (iPSC cells), and NK cell lines, such as, but not limited to, the NK-92, NK101, KHYG-1, YT, NK-YS, YTS, HANK-1, NKL, and NK3.3 cell lines.

While the immune cells of the present disclosure may be targeted to any combination of antigens, exemplary antigens for the CAR include but are not limited to CD70. In particular aspects, the immune cells are dually targeted to an antigen combination including CD70 and CD33 (e.g., for AML), CD70 and CD123 (e.g., for AML), CD70 and CLL1 (e.g., for AML), CD70 and CD96 (e.g., for AML); CD70 and Flt3 (e.g., for AML); CD70 and CD19 (e.g., for B cell malignancies); CD70 and CD22 (e.g., for B cell malignancies); CD70 and CD20 (e.g., for B cell malignancies); CD70 and CD79a (e.g., for B cell malignancies); CD70 and CD79b (e.g., for B cell malignancies); CD70 and FcRH5 (e.g., for B cell malignancies); CD70 and BCMA (e.g., for multiple myeloma); CD70 and GPRC5D (e.g., for multiple myeloma); CD70 and FcRL5 (e.g., for multiple myeloma); CD70 and CD138 (e.g., for multiple myeloma); CD70 and CD96 (e.g., for RCC); CD70 and HAVCR1 (e.g., for RCC); CD70 and EGFR (e.g., for RCC).

In further embodiments, the NK cells provided herein are genetically modified (e.g., transduced with a vector) to express two CARs. Examples of target antigens include, but are not limited to CD96 and CD33; CD123 and CD33; CD19 and ROR1; CD38 and BCMA; BCMA and GPRC5D; BCMA and CD138; CD19 and CD22, CD79a and CD22; CD37 and CXCR5. These NK cells have dual specificity and may further be engineered to express an exogenous polypeptide comprising IL-15 or another cytokine which enhances the in vivo persistence of the NK-cells (e.g., without additional exogenous cytokine support). In addition, the expression of two CARs provides the NK cells increased specificity by limiting the off-target toxicity of the cells, such that a signal is only provided to the NK cells to kill when the cells contact both antigens expressed on a tumor, as well as enhanced in vivo proliferation and persistence. Thus, normal cells that express only one antigen may not be targeted by the NK cells.

Genetic reprogramming of immune cells, such as NK cells and T cells, for adoptive cancer immunotherapy has clinically relevant applications and benefits such as 1) innate anti-tumor surveillance without prior need for sensitization 2) allogeneic efficacy without graft versus host reactivity in the case of NK cells and 3) direct cell-mediated cytotoxicity and cytolysis of target tumors. Accordingly, the present disclosure also provides methods for treating immune-related disorders, such as cancer, comprising adoptive cell immunotherapy with any of the engineered immune cells provided herein.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” or “additional” may mean at least a second or more.

As used herein, the term “about” is used to indicate that a value includes the inherent variation of error for the device, the method being employed to determine the value, or the variation that exists among the study subjects.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. For example, in some embodiments, if a full-length polypeptide binds a given ligand, a portion of that full-length polypeptide also binds to the same ligand.

The terms “protein” and “polypeptide” are used interchangeably herein.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either in vitro or in vivo.

A “plasmid,” a common type of a vector, is an extra-chromosomal DNA molecule separate from the chromosomal DNA that is capable of replicating independently of the chromosomal DNA. In certain cases, it is circular and double-stranded.

A “gene,” “polynucleotide,” “coding region,” “sequence,” “segment,” “fragment,” or “transgene” that “encodes” a particular protein, is a section of a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to a RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and a functional effector element) are connected in such a way as to permit transcription of the nucleic acid molecule.

The term “homology” refers to the percent of identity between the nucleic acid residues of two polynucleotides or the amino acid residues of two polypeptides. The correspondence between one sequence and another can be determined by techniques known in the art. For example, homology can be determined by a direct comparison of the sequence information between two polypeptides by aligning the sequence information and using readily available computer programs. Two polynucleotide (e.g., DNA) or two polypeptide sequences are “substantially homologous” to each other when at least about 80%, preferably at least about 90%, and most preferably at least about 95% of the nucleotides, or amino acids, respectively match over a defined length of the molecules, as determined using the methods above.

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell, and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPScs,” “iPSCs,” or “iPS cells.”

An “embryonic stem (ES) cell” is an undifferentiated pluripotent cell which is obtained from an embryo in an early stage, such as the inner cell mass at the blastocyst stage, or produced by artificial means (e.g., nuclear transfer) and can give rise to any differentiated cell type in an embryo or an adult, including germ cells (e.g., sperm and eggs).

“Induced pluripotent stem cells” (“iPScs,” “iPSCs” or “iPS cells”) are cells generated by reprogramming a somatic cell by expressing or inducing expression of a combination of factors (herein referred to as reprogramming factors). iPS cells can be generated using fetal, postnatal, newborn, juvenile, or adult somatic cells. In certain embodiments, factors that can be used to reprogram somatic cells to pluripotent stem cells include, for example, Oct4 (sometimes referred to as Oct 3/4), Sox2, c-Myc, Klf4, Nanog, and Lin28. In some embodiments, somatic cells are reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Hematopoietic progenitor cells” or “hematopoietic precursor cells” refers to cells which are committed to a hematopoietic lineage but are capable of further hematopoietic differentiation and include hematopoietic stem cells, multipotential hematopoietic stem cells, common myeloid progenitors, megakaryocyte progenitors, erythrocyte progenitors, and lymphoid progenitors. Hematopoietic stem cells (HSCs) are multipotent stem cells that give rise to all the blood cell types including myeloid (monocytes and macrophages, granulocytes (neutrophils, basophils, eosinophils, and mast cells), erythrocytes, megakaryocytes/platelets, dendritic cells), and lymphoid lineages (T cells, B cells, NK cells).

A “multilymphoid progenitor” (MLP) is defined to describe any progenitor that gives rise to all lymphoid lineages (B, T, and NK cells), but that may or may not have other (myeloid) potentials and is CD45RA⁺, /CD10⁺/CD7\ Any B, T, and NK progenitor can be referred to as an MLP. A “common myeloid progenitor” (CMP) refers to CD45RA⁻/CD135⁺/CD10⁻/CD7⁻ cells that can give rise to granulocytes, monocytes, megakaryocytes, and erythrocytes.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

“Programming” is a process that alters the type of progeny a cell can produce. For example, a cell has been programmed when it has been altered so that it can form progeny of at least one new cell type, either in culture or in vivo, as compared to what it would have been able to form under the same conditions without programming. This means that after sufficient proliferation, a measurable proportion of progeny having phenotypic characteristics of the new cell type are observed, if essentially no such progeny could form before programming; alternatively, the proportion having characteristics of the new cell type is measurably more than before programming. This process includes differentiation, dedifferentiation and transdifferentiation.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type. Various cell types can be used as feeder cells including, but not limited to, peripheral blood derived cells (e.g., autologous peripheral blood mononuclear cells), transformed leukemia cells (e.g., erythroleukemic cell lines such as the K562 cell line), certain Wilm's tumor cell lines (e.g., HFWT), endometrial tumor cells (HHUA), melanoma cells (e.g., HMV-II), hepatoblastoma cells (e.g., HuH-6), lung small cell carcinoma cells (e.g., Lu-130 and Lu-134-A), neuroblastoma cells (e.g., NB19 and NB69), embryonal carcinoma testis cells (e.g., NEC14), cervical carcinoma cells (TCO-2), neuroblastoma cells (e.g., TNB1), Epstein Barr virus transformed lymphocyte contiuous line (EBV-LCL), CD4+ T cells, T cell lymphoma cell lines (e.g., HUT78), among others. In some embodiments, the feeder cells may be inactivated when being co-cultured with other cells by irradiation or treatment with an anti-mitotic agent such as mitomycin. In some embodiments, the feeder cells comprise a modification to increase expression of one or more factors capable of increasing immune cell activation and/or proliferation, including, e.g., a co-stimulatory molecule such as CD40L, OX40L, CD86, CD137L, CD80 or CD83, a cytokine such as IL-21, IL-15, membrane-bound IL-21, membrane-bound IL-15, IL-7, IL-18 and IL-2, and/or an antigen.

As used herein, a “feeder-free” (FF) environment refers to an environment such as a culture condition, cell culture or culture media which is essentially free of feeder cells, and/or which has not been pre-conditioned by the cultivation of feeder cells.

As used herein, the term “subject” or “subject in need thereof” refers to a mammal, preferably a human being, male or female at any age that is in need of a therapeutic intervention, a cell transplantation or a tissue transplantation. Typically, the subject is in need of therapeutic intervention, cell or tissue transplantation (also referred to herein as recipient) due to a disorder or a pathological or undesired condition, state, or syndrome, or a physical, morphological or physiological abnormality which is amenable to treatment via therapeutic intervention, or cell or tissue transplantation.

As used herein, a “disruption” or “alteration” in reference to a gene refers to a homologous recombination event with a nucleic acid molecule (e.g., an endogenous gene sequence) which results in elimination or reduction of expression of one or more gene products encoded by the subject gene in a cell, compared to the level of expression of the gene product in the absence of the disruption. Exemplary gene products include mRNA and protein products encoded by the subject gene. Alteration in some cases is transient or reversible and in other cases is permanent. Alteration, in some cases, is of a functional or full-length protein or mRNA, despite the fact that a truncated or nonfunctional product may be produced. In some embodiments herein, gene activity or function, as opposed to expression, is disrupted. Gene alteration is generally induced by artificial methods, i.e., by addition or introduction of a compound, molecule, complex, or composition, and/or by alteration of nucleic acid of (or associated) with the gene, such as at the DNA level. Exemplary methods for gene alteration include gene silencing, knockdown, knockout, and/or gene alteration techniques, such as gene editing. Examples of gene editing methods include CRISPR/Cas systems, meganuclease systems, Zinc Finger Protein (ZFP) and Zinc Finger Nuclease (ZFN) systems and/or transcription activator-like protein (TAL), transcription activator-like effector protein (TALE) or TALE nuclease protein (TALEN) systems. Examples of gene alteration also include antisense technology, such as RNAi, siRNA, shRNA, tandem shRNAs, and/or ribozymes, which generally result in transient reduction of expression, as well as gene editing techniques which result in targeted gene inactivation or alteration, e.g., by induction of breaks and/or homologous recombination. Examples include insertions, mutations, and deletions. The alterations typically result in the repression and/or complete absence of expression of a normal or “wild type” product encoded by the gene. Examples such gene alterations are insertions, frameshift and mis sense mutations, deletions, substitutions, knock-in, and knock-out of the gene or part of the gene, including deletions of the entire gene. Such alterations can occur in the coding region, e.g., in one or more exons, resulting in the inability to produce a full-length product, functional product, or any product, such as by insertion of a stop codon. Such alterations may also occur by alterations in the promoter or enhancer or other region affecting activation of transcription, so as to prevent transcription of the gene. Gene alterations include gene targeting, including targeted gene inactivation by homologous recombination.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a NK cell, B cell, or a T cell, or innate immune cell to a stimulus. In some embodiments, the response is specific for a particular antigen (an “antigen-specific response”).

As used herein, the term “antigen” is a molecule capable of being bound by an antibody or T cell receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

An “autoimmune disease” refers to a disease in which the immune system produces an immune response (for example, a B cell or a T cell response) against an antigen that is part of the normal host (that is, an autoantigen), with consequent injury to tissues. An autoantigen may be derived from a host cell or may be derived from a commensal organism such as the micro-organisms (known as commensal organisms) that normally colonize mucosal surfaces.

The term “Graft-Versus-Host Disease (GVHD)” refers to a common and serious complication of bone marrow or other tissue transplantation wherein there is a reaction of donated immunologically competent lymphocytes against a transplant recipient's own tissue. GVHD is a possible complication of any transplant that uses or contains stem cells from either a related or an unrelated donor. In some embodiments, the GVHD is chronic GVHD (cGVHD).

A “parameter of an immune response” is any particular measurable aspect of an immune response, including, but not limited to, cytokine secretion (IL-6, IL-10, IFN-γ, etc.), chemokine secretion, altered migration or cell accumulation, immunoglobulin production, dendritic cell maturation, regulatory activity, number of immune cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of ³H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or “treatment of a disease or condition” refers to executing a protocol or treatment plan, which may include administering one or more drugs or active agents (e.g., genetically engineered immune cells, e.g., genetically engineered NK cells) to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. In addition, “treating” or “treatment” does not require complete alleviation of signs or symptoms, and does not require a cure.

The term “therapeutic benefit” or “therapeutically effective” as used throughout this application refers to anything that promotes or enhances the well-being of the subject with respect to the medical treatment of this condition. This includes, but is not limited to, a reduction in the frequency, severity, or rate of progression of the signs or symptoms of a disease. For example, treatment of cancer may involve, for example, a reduction in the size of a tumor, a reduction in the invasiveness of a tumor, reduction in the growth rate of the cancer, or a reduction in the rate of metastasis or recurrence. Treatment of cancer may also refer to prolonging survival of a subject with cancer.

“Antigen recognition moiety” or “antigen recognition domain” refers to a molecule or portion of a molecule that specifically binds to an antigen. In some embodiments, the antigen recognition moiety is an antibody, antibody like molecule or fragment thereof and the antigen is a tumor antigen.

“Antibody” as used herein refers to monoclonal or polyclonal antibodies. An antibody can be an IgG1, IgG2, IgG3, IgG4, IgM, IgE, or IgA antibody. In some embodiments, an antibody can be a human or humanized antibody.

“Antibody like molecules” may be for example proteins that are members of the Ig-superfamily which are able to selectively bind a partner.

The terms “fragment of an antibody,” “antibody fragment,” “functional fragment of an antibody,” and “antigen-binding portion” are used interchangeably herein to mean one or more fragments or portions of an antibody that retain the ability to specifically bind to an antigen (see, generally, Holliger et al. (2005) Nat. Biotech. 23(9): 1126-9). The antibody fragment desirably comprises, for example, one or more CDRs, the variable region (or portions thereof), the constant region (or portions thereof), or combinations thereof. Examples of antibody fragments include, but are not limited to, (i) a Fab fragment; (ii) a F(ab′)₂ fragment; (iii) a Fv fragment; (iv) a single chain Fv (scFv); and (v) a diabody.

“Chimeric Antigen Receptor” or “CAR” (also known as artificial cell receptors, chimeric cell receptors, or chimeric immunoreceptors) are engineered receptors, which graft a selected specificity onto an immune effector cell. CARs may be employed to impart the specificity of a monoclonal antibody onto an immune cell (e.g., a T cell or an NK cell), thereby allowing a large number of specific immune cells to be generated, for example, for use in adoptive cell therapy. In some embodiments, CARs direct specificity of the immune cell to a tumor-associated antigen. CARs typically have an extracellular domain (ectodomain), which comprises an antigen-binding domain and a stalk region, a transmembrane domain and one or more intracellular (endodomain) domain(s). In some examples, CARs comprise fusions of single-chain variable fragments (scFv) derived from monoclonal antibodies, fused to CD3-zeta a transmembrane domain and endodomain. The specificity of other CAR designs may be derived from ligands of receptors (e.g., peptides) or from pattern-recognition receptors, such as Dectins. In some examples, the spacing of the antigen-recognition domain can be modified to reduce activation-induced cell death. In some examples, CARs comprise domains for additional co-stimulatory signaling, such as CD3zeta, FcR, CD27, CD28, 4-1BB, CD137, DAP10, DAP12, 2B4, ICOS, OX40 and/or OX40L. In some embodiments, molecules can be co-expressed with the CAR, including co-stimulatory molecules, reporter genes for imaging (e.g., for positron emission tomography), safety switch proteins, homing receptors, chemokines, chemokine receptors, cytokines, cytokine receptors, and a TGFbeta signal converter.

A “stalk” region, which encompasses the terms “spacer region” or “hinge domain” or “hinge” is used to link the antigen-binding domain to the transmembrane domain. As used herein, the term “stalk region” generally means any polypeptide that functions to link the transmembrane domain to, either the extracellular domain or, the cytoplasmic domain in the polypeptide chain of a CAR. In embodiments, the stalk region is flexible enough to allow the antigen-binding domain to orient in different directions to facilitate antigen recognition. In some embodiments, the hinge domain is derived from IgG1 the CH2CH3 region of immunoglobulin, and portions of CD3. In some embodiments, the stalk region is a CD8alpha (also referred to herein as CD8a and CD8a) hinge (SEQ ID NO: 619). The term “functional portion,” when used in reference to a CAR, refers to any part or fragment of a CAR described herein, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example, at least about 10%, 25%, 30%, 50%, 68%, 80%, 90%, 95%, or more, of the parent CAR.

The term “functional variant,” as used herein, refers to a polypeptide, or a protein having substantial or significant sequence identity or similarity to the reference polypeptide, and retains the biological activity of the reference polypepide of which it is a variant. Functional variants encompass, for example, those variants of the CAR described herein (the parent CAR) that retain the ability to recognize target cells to a similar extent, the same extent, or to a greater extent, as the parent CAR. In reference to a nucleic acid sequence encoding the parent CAR, a nucleic acid sequence encoding a functional variant of the CAR can be for example, at least about 10% identical, at least about 25% identical, at least about 30% identical, at least about 50% identical, at least about 65% identical, at least about 70% identical, at least about 75% identical, at least about 80% identical, at least about 85% identical, at least about 90% identical, at least about 95% identical, or at least about 99% identical to the nucleic acid sequence encoding the parent CAR.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous biocompatible solvents (e.g., saline solutions, phosphate buffered saline, parenteral vehicles, such as sodium chloride, Ringer's dextrose, etc.), antioxidants, preservatives (e.g., antibacterial or antifungal agents, anti-oxidants, chelating agents, and inert gases), isotonic agents, such like materials and combinations thereof, as would be known to one of ordinary skill in the art. The pH and exact concentration of the various components in a pharmaceutical composition are adjusted according to well-known parameters.

The term “T cell” refers to T lymphocytes, and includes, but is not limited to, γ:δ⁺ T cells, NK T cells, CD4⁺ T cells and CD8⁺ T cells. CD4⁺ T cells include THO, Tₕ1 and TH2 cells, as well as regulatory T cells (T_{reg}). There are at least three types of regulatory T cells: CD4⁺CD25⁺ T_{reg}, CD25 T_{H}3 T_{reg}, and CD25 T_{R} 1 T_{reg}. “Cytotoxic T cell” refers to a T cell that can kill another cell. The majority of cytotoxic T cells are CD8⁺ MHC class I-restricted T cells, however some cytotoxic T cells are CD4⁺. In preferred embodiments, the T cell of the present disclosure is CD4⁺ or CD8⁺.

The activation state of a T cell defines whether the T cell is “resting” (i.e., in the G₀ phase of the cell cycle) or “activated” to proliferate after an appropriate stimulus such as the recognition of its specific antigen, or by stimulation with OKT3 antibody, PHA or PMA, etc. The “phenotype” of the T cell (e.g., naive, central memory, effector memory, lytic effectors, help effectors (TH1 and TH2 cells), and regulatory effectors), describes the function the cell exerts when activated. A healthy donor has T cells of each of these phenotypes, and which are predominately in the resting state. A naive T cell will proliferate upon activation, and then differentiate into a memory T cell or an effector T cell. It can then assume the resting state again, until it gets activated the next time, to exert its new function and may change its phenotype again. An effector T cell will divide upon activation and antigen-specific effector function.

“Natural killer T cells” (“NKT cells”), not to be confused with natural killer cells of the innate immune system, bridge the adaptive immune system with the innate immune system. Unlike conventional T cells that recognize peptide antigens presented by major histocompatibility complex (WIC) molecules, NKT cells recognize glycolipid antigen presented by a molecule called CD1d. Once activated, these cells can perform functions ascribed to both Th and Tc cells (i.e., cytokine production and release of cytolytic/cell killing molecules). They are also able to recognize and eliminate some tumor cells and cells infected with herpes viruses.

“Natural killer cells” (“NK cells”) are a type of cytotoxic lymphocyte of the innate immune system. In some instances, NK cells provide a first line defense against viral infections and/or tumor formation. NK cells can detect MHC presented on infected or cancerous cells, triggering cytokine release, and subsequently induce lysis and apoptosis. NK cells can further detect stressed cells in the absence of antibodies and/or MHC, thereby allowing a rapid immune response.

“AML,” as used herein, refers to acute myelogenous leukemia, also known as acute myelocytic leukemia, acute myeloid leukemia, acute granulocytic leukemia, and acute non-lymphocytic leukemia. AML is differentiated from the other main forms of leukemia because it is a rapidly progressing malignancy of the myeloid lineage. AML has eight different subtypes based on the cell type that the leukemia developed from. One method of classifying the subtypes is the WHO classification method (Dohner et al. Blood 129: 424-47, 2017). The term “AML” therefore refers to all subtypes, including myeloblastic (MO) on special analysis, myeloblastic (MI) without maturation, myeloblastic (M2) with maturation, promyeloctic (M3), myelomonocytic (M4), monocytic (M5), erythroleukemia (M6) and megakaryocytic (M7).

“Relapsed AML” refers to patients who have experienced a recurrence following an interval of remission of AML.

“Refractory AML” refers to patients whose disease does not respond to the first cycle of initial standard induction therapy (e.g, anthracycline and/or cytarabine-based therapy). In some embodiments, “refractory AML” refers to patients who lack remission following initial therapy. In some embodiments, “refractory AML” refers to subjects whose disease does not respond to one or two or more cycles of standard induction therapy.

The term “antigen presenting cells” or “APCs” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B cells, endothelial cells, activated T cells, and dendritic cells; or other molecules, naturally occurring or synthetic, such as purified MHC Class I molecules complexed to 2-microglobulin.

The term “culturing” refers to the in vitro maintenance, differentiation, and/or propagation of cells in suitable media. By “enriched” is meant a composition comprising cells present in a greater percentage of total cells than is found in the tissues where they are present in an organism.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence, number, and/or rate of development of metastases, reducing solid tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

As used herein, the term “click reaction” refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic by-products that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used. In some embodiments, the click reaction is fast, specific, and high yield.

As used herein, the term “click handle” refers to a chemical moiety that is capable of reacting with a second click handle in a click reaction to produce a click signature. In embodiments, a click handle is comprised by a coupling reagent, and the coupling reagent may further comprise a substrate reactive moiety.

As used herein, the term “sortase,” refers to an enzyme which catalyzes a transpeptidation reaction between a sortase recognition motif and a sortase acceptor motif. As used herein, the transpeptidation reaction between a sortase recognition motif and a sortase acceptor motif is termed a “sortase-mediated transpeptidation reaction”. Various sortases from prokaryotic organisms have been identified. In some embodiments, the sortase catalyzes a reaction to conjugate the C-terminus of a first moiety containing a sortase recognition motif to the N-terminus of a second moiety containing a sortase acceptor motif by a peptide bond. In some embodiments, the sortase catalyzes a reaction to couple a first moiety to a second moiety by a peptide bond. In some embodiments, sortase mediated transfer is used to couple the N-terminus of a first polypeptide, e.g., an extracellular binding domain of a protein on an NK cell to the N-terminus of a second polypeptide, e.g., an antigen binding domain, to the N terminus of a second polypeptide. In such embodiments, sortase mediated transfer is used to attach a coupling moiety, e.g., a “click” handle, to the N-terminus of each polypeptide, wherein the coupling moieties mediate coupling of the polypeptides. In an embodiment the first polypeptide is an extracellular binding domain, e.g., an antigen binding domain, comprising a sortase acceptor motif, and the second polypeptide is a transmembrane polypeptide comprising an extracellular N-terminal sortase acceptor motif, a transmembrane domain, and an intracellular signaling domain. Sortase mediated transfer is used to attach a coupling moiety, e.g., a click handle, to each polypeptide.

“Sortase acceptor motif,” as that term is used herein, refers to a moiety that that acts as an acceptor for the sortase-mediated transfer of a polypeptide, from the sortase, to the sortase acceptor motif. In an embodiment the sortase acceptor motif is located at the N terminus of a polypeptide. In an embodiment the transferred polypeptide is linked by a peptide bond at its C terminus to the N terminal residue of the sortase acceptor motif. N-terminal acceptor motifs include Gly-[Gly]ₙ- (SEQ ID NO: 2), wherein n=0-5 and Ala-[Ala]ₙ- (SEQ ID NO: 3), wherein n=0-5.

“Sortase recognition motif,” as that term is used herein, refers to polypeptide which, upon cleavage by a sortase, e.g., a, forms a thioester bond with the sortase. In an embodiment, sortase cleavage occurs between T and G/A. In an embodiment the peptide bond between T and G/A is replaced with an ester bond to the sortase.

“Sortase transfer signature,” as that term is used herein, refers to the portion of a sortase recognition motif and the portion of a sortase acceptor motif remaining after the reaction that couples the former to the latter. In an embodiment, wherein the sortase recognition motif is LPXTG/A (SEQ ID NO: 4) and wherein the sortase acceptor motif is GG, the resultant sortase transfer signature after sortase-mediated reaction comprises LPXTGG (SEQ ID NO: 5).

An “inhibitory extracellular domain,” as that term is used herein, refers to polypeptide comprising an extracellular domain of an inhibitory molecule. Normally, binding to its counterligand has an inhibitory effect on the generation of an immune effector response (e.g., NK cell activation or response). When linked, e.g., fused to an intracellular signaling domain, it redirects an interaction that normally inhibits the generation of an immune effector response into one that promotes an immune effector response.

“Inhibitory molecule,” as that term is used herein, refers to a molecule, e.g., an endogenous molecule, of a cell described herein that upon binding to its cognate counter ligand on a target cell, minimizes, e.g., suppresses or inhibits, an immune effector response (e.g., NK cell activation or response). Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and a TGF beta receptor (e.g., TGFBRI and TGFBRII).

### II. Immune Cells

Certain embodiments of the present disclosure concern immune cells (e.g., NK cells or T cells) having decreased levels (e.g., about a 1% to about a 100%, about a 1% to about a 95%, about a 1% to about a 90%, about a 1% to about a 85%, about a 1% to about a 80%, about a 1% to about a 75%, about a 1% to about a 70%, about a 1% to about a 65%, about a 1% to about a 60%, about a 1% to about a 55%, about a 1% to about a 50%, about a 1% to about a 45%, about a 1% to about a 40%, about a 1% to about a 35%, about a 1% to about a 30%, about a 1% to about a 25%, about a 1% to about 20%, about a 1% to about a 15%, about a 1% to about a 10%, about a 1% to about a 5%, about a 5% to about a 100%, about a 5% to about a 95%, about a 5% to about a 90%, about a 5% to about a 85%, about a 5% to about a 80%, about a 5% to about a 75%, about a 5% to about a 70%, about a 5% to about a 65%, about a 5% to about a 60%, about a 5% to about a 55%, about a 5% to about a 50%, about a 5% to about a 45%, about a 5% to about a 40%, about a 5% to about a 35%, about a 5% to about a 30%, about a 5% to about a 25%, about a 5% to about 20%, about a 5% to about a 15%, about a 5% to about a 10%, about a 10% to about a 100%, about a 10% to about a 95%, about a 10% to about a 90%, about a 10% to about a 85%, about a 10% to about a 80%, about a 10% to about a 75%, about a 10% to about a 70%, about a 10% to about a 65%, about a 10% to about a 60%, about a 10% to about a 55%, about a 10% to about a 50%, about a 10% to about a 45%, about a 10% to about a 40%, about a 10% to about a 35%, about a 10% to about a 30%, about a 10% to about a 25%, about a 10% to about 20%, about a 10% to about a 15%, about a 15% to about a 100%, about a 15% to about a 95%, about a 15% to about a 90%, about a 15% to about a 85%, about a 15% to about a 80%, about a 15% to about a 75%, about a 15% to about a 70%, about a 15% to about a 65%, about a 15% to about a 60%, about a 15% to about a 55%, about a 15% to about a 50%, about a 15% to about a 45%, about a 15% to about a 40%, about a 15% to about a 35%, about a 15% to about a 30%, about a 15% to about a 25%, about a 15% to about 20%, about a 20% to about a 100%, about a 20% to about a 95%, about a 20% to about a 90%, about a 20% to about a 85%, about a 20% to about a 80%, about a 20% to about a 75%, about a 20% to about a 70%, about a 20% to about a 65%, about a 20% to about a 60%, about a 20% to about a 55%, about a 20% to about a 50%, about a 20% to about a 45%, about a 20% to about a 40%, about a 20% to about a 35%, about a 20% to about a 30%, about a 20% to about a 25%, about a 25% to about a 100%, about a 25% to about a 95%, about a 25% to about a 90%, about a 25% to about a 85%, about a 25% to about a 80%, about a 25% to about a 75%, about a 25% to about a 70%, about a 25% to about a 65%, about a 25% to about a 60%, about a 25% to about a 55%, about a 25% to about a 50%, about a 25% to about a 45%, about a 25% to about a 40%, about a 25% to about a 35%, about a 25% to about a 30%, about a 30% to about a 100%, about a 30% to about a 95%, about a 30% to about a 90%, about a 30% to about a 85%, about a 30% to about a 80%, about a 30% to about a 75%, about a 30% to about a 70%, about a 30% to about a 65%, about a 30% to about a 60%, about a 30% to about a 55%, about a 30% to about a 50%, about a 30% to about a 45%, about a 30% to about a 40%, about a 30% to about a 35%, about a 35% to about a 100%, about a 35% to about a 95%, about a 35% to about a 90%, about a 35% to about a 85%, about a 35% to about a 80%, about a 35% to about a 75%, about a 35% to about a 70%, about a 35% to about a 65%, about a 35% to about a 60%, about a 35% to about a 55%, about a 35% to about a 50%, about a 35% to about a 45%, about a 35% to about a 40%, about a 40% to about a 100%, about a 40% to about a 95%, about a 40% to about a 90%, about a 40% to about a 85%, about a 40% to about a 80%, about a 40% to about a 75%, about a 40% to about a 70%, about a 40% to about a 65%, about a 40% to about a 60%, about a 40% to about a 55%, about a 40% to about a 50%, about a 40% to about a 45%, about a 45% to about a 100%, about a 45% to about a 95%, about a 45% to about a 90%, about a 45% to about a 85%, about a 45% to about a 80%, about a 45% to about a 75%, about a 45% to about a 70%, about a 45% to about a 65%, about a 45% to about a 60%, about a 45% to about a 55%, about a 45% to about a 50%, about a 50% to about a 100%, about a 50% to about a 95%, about a 50% to about a 90%, about a 50% to about a 85%, about a 50% to about a 80%, about a 50% to about a 75%, about a 50% to about a 70%, about a 50% to about a 65%, about a 50% to about a 60%, about a 50% to about a 55%, about a 55% to about a 100%, about a 55% to about a 95%, about a 55% to about a 90%, about a 55% to about a 85%, about a 55% to about a 80%, about a 55% to about a 75%, about a 55% to about a 70%, about a 55% to about a 65%, about a 55% to about a 60%, about a 60% to about a 100%, about a 60% to about a 95%, about a 60% to about a 90%, about a 60% to about a 85%, about a 60% to about a 80%, about a 60% to about a 75%, about a 60% to about a 70%, about a 60% to about a 65%, about a 65% to about a 100%, about a 65% to about a 95%, about a 65% to about a 90%, about a 65% to about a 85%, about a 65% to about a 80%, about a 65% to about a 75%, about a 65% to about a 70%, about a 70% to about a 100%, about a 70% to about a 95%, about a 70% to about a 90%, about a 70% to about a 85%, about a 70% to about a 80%, about a 70% to about a 75%, about a 75% to about a 100%, about a 75% to about a 95%, about a 75% to about a 90%, about a 75% to about a 85%, about a 75% to about a 80%, about a 80% to about a 100%, about a 80% to about a 95%, about a 80% to about a 90%, about a 80% to about a 85%, about a 85% to about a 100%, about a 85% to about a 95%, about a 85% to about a 90%, about a 90% to about a 100%, about a 90% to about a 95%, or about a 95% to about a 100%, decrease) of CD70 (e.g., protein or mRNA) as compared to an immune cell of the same type (e.g., an NK cell or a T cell) that is not contacted with the CD70 inhibitor (e.g., a wild-type NK cell or a population of wild-type NK cells), e.g., produced using any of the exemplary methods described herein. In some embodiments, the genetically engineered immune cells (e.g., genetically engineered NK cells or genetically engineered T cells) express a CAR (e.g., one or more of any of the exemplary CARs described herein). In some embodiments, the genetically engineered immune cells comprise at least one exogenous polypeptide. In some embodiments, the at least one exogenous polypeptide is selected from the group of: a cytokine, a chemokine, a ligand, a receptor, a monoclonal antibody, a bispecific T cell engager, a peptide, or an enzyme, a subunit or a portion of the foregoing, or any combination of the foregoing. In some embodiments, the at least one exogenous polypeptide comprises a cytokine and wherein the cytokine comprises IL-15, membrane-bound IL-15 (mbIL-15), IL-2, membrane-bound IL-2, IL-12, membrane-bound IL-12, IL-18, membrane-bound IL-18, IL-21, membrane-bound IL-21, p40, LIGHT, CD40L, FLT3L, 4-1BBL, or FASL. In some embodiments, the at least one exogenous polypeptide comprises a receptor selected from the group of: CSF-1R, a CXC chemokine receptor, a CC chemokine receptor, a CX3C chemokine receptor, a XC chemokine receptor, or a chemokine-binding fragment thereof. In some embodiments, the at least one exogenous polypeptide is a protein that overcomes immunosuppression of the tumor microenvironment (e.g., a TGFbeta signal converter or a TGFbeta decoy receptor). In some embodiments, the at least one exogenous polypeptide comprises a safety switch protein.

In some embodiments, the immune cells express a chimeric antigen receptor (CAR). The immune cells may be T cells (e.g., regulatory T cells, CD4⁺ T cells, CD8⁺ T cells, or gamma-delta T cells), NK cells, invariant NK cells, NKT cells, stem cells (e.g., mesenchymal stem cells (MSCs) or induced pluripotent stem (iPSC) cells). In some embodiments, the cells are monocytes or granulocytes, e.g., myeloid cells, macrophages, neutrophils, dendritic cells, mast cells, eosinophils, and/or basophils. Also provided herein are methods of producing and engineering the immune cells as well as methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. The immune cells may be enriched/purified from any tissue where they reside including, but not limited to, blood (including blood collected by blood banks or cord blood banks), spleen, bone marrow, tissues removed and/or exposed during surgical procedures, and tissues obtained via biopsy procedures. Tissues/organs from which the immune cells are enriched, isolated, and/or purified may be isolated from both living and non-living subjects, wherein the non-living subjects are organ donors. The isolated immune cells may be used directly, or they can be stored for a period of time, such as by freezing. In some embodiments, the immune cells are isolated from blood, such as peripheral blood or cord blood. In some embodiments, immune cells isolated from cord blood have enhanced immunomodulation capacity, such as measured by CD4-positive or CD8-positive T cell suppression. In specific aspects, the immune cells are isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune cell activity. Thus, the cells will be autologous to the subject in need of therapy. Alternatively, the population of immune cells can be obtained from a donor. The immune cell population can be harvested from the peripheral blood, cord blood, bone marrow, spleen, or any other organ/tissue in which immune cells reside in said subject or donor. The immune cells can be isolated from a pool of subjects and/or donors, such as from pooled cord blood.

When the population of immune cells is obtained from a donor distinct from the subject, the donor is preferably allogeneic, provided the cells obtained are subject-compatible in that they can be introduced into the subject. Allogeneic donor cells may or may not be human leukocyte antigen (HLA)-compatible. To be rendered subject-compatible, allogeneic cells can be treated to reduce immunogenicity.

### 1. NK Cells

In some embodiments, the immune cells are NK cells. NK cells are a subpopulation of lymphocytes that have spontaneous cytotoxicity against a variety of tumor cells, virus-infected cells, and some normal cells in the bone marrow and thymus. NK cells can be detected by specific surface markers, such as CD16, CD56, and CD8 in humans. NK cells do not express T cell antigen receptors, the pan T marker CD3, or surface immunoglobulin B cell receptors.

Expansion of NK Cells

NK cells can be expanded by various methods known in the art. In some instances, NK cells can be expanded or enriched from large volumes of peripheral blood, such as an apheresis products (e.g., mobilized PBSCs or unmobilized PBSCs). In other instances, NK cells can be expanded or enriched from smaller number of blood or stem cells. Expansion of NK cells from apharesis products are described, for example, in Lapteva et al. Crit. Rev. Oncog. 19:121-132, 2014; Miller et al. Blood 105(8):3051-7, 2005; Lapteva et al. Cytotherapy 14(9):1131-43, 2012; Spanholtz et al. PLoS One 6(6):e20740, 2011; Knorr et al. Stem Cells Transl. Med. 2(4):274-83, 2013; Pfeiffer et al. Leukemia 26(11):2435-9,2012; Shi et al. Br. J. Haematol. 143(5):641-53, 2008; Passweg et al. Leukemia 18(11):1835-8, 2004; Koehl et al. Klin. Padiatr. 217(6):345-50, 2005; and Klingemann et al. Transfusion 53(2):412-8, 2013. Approaches that generate NK cells for allogeneic use aim to minimize CD3⁺ T-lymphocyte populations that may cause graft-versus-host disease (GVHD). This often involves depletion of CD3⁺ T cells, which increases the total number of starting cells required, particularly if depletion is performed at the end of the manufacturing procedure. Most protocols, therefore, use apheresis products (1×10⁹-20×10⁹ mononuclear cells) as the starting material; however, expansion from other sources such as buffy coats, cord blood, and embryonic stem cells is also possible. NK cells in peripheral blood and apheresis products can be detected by flow cytometry as CD45⁺CD56⁺CD3⁻ cells. In some instances, NK cells can be enriched from apheresis products by one or two rounds of depletion of CD3⁺ T cells using magnetic beads (e.g., CLINIMACS magnetic beads) coated with anti-CD3 antibody (e.g., CLINIMACS CD3 reagent) with or without overnight activation using IL-2 or IL-15. This method can produce up to 2×10⁹NK cells with approximately 20% purity, while contaminating CD19⁺ B cells, and CD14⁺ monocytes can comprise greater than 50% of the product. Additional depletion of CD19⁺ B cells with anti-CD19 antibody-coated magnetic beads (e.g., CliniMACS CD19 reagent) can further improve the purity of the NK cells, resulting in an average of 40% CD56⁺CD3⁻ in the final product. Alternatively, NK cells can be enriched by isolating CD56⁺ cells using anti-CD56 monoclonal antibody (e.g., CLINIMACS CD56 reagent) with or without CD3⁺ T cell depletion. Without CD3⁺ T cell depletion, this method can yield more than 95% NK cell purity while retaining CD56⁺CD3⁺ natural killer like T (NKT) cells, which also may contribute to anti-tumor immune responses, whereas the inclusion of CD3⁺ T-cell depletion can yield up to 99% purity.

In some instances, NK cells can be expanded using feeder cell-based technology. Such methods are described, for example, in Berg et al. Cytotherapy 11(3):341-55, 2009; Lapteva et al. 2012, supra; and Lapteva et al. Crit. Rev. Oncog. 19:121-132, 2014. Because therapeutic use of NK cells demand high NK cell doses and often several infusions, one apheresis product may not contain sufficient numbers of NK cells. Therefore, technically complicated NK cell expansion protocols have been developed. Expansion of NK cells with either IL-2 or IL-15 or both to produce 1,000-fold expansion requires a culture period of up to 12 weeks. By contrast, feeder cell-based NK expansion approaches are rapid and robust, as large numbers of NK cells become available for infusion within 10-14 days (Lapteva et al., 2012, supra). Feeder-cell methods generally require cytokines as well as irradiated feeder cells, such as EBV-LCLs or genetically modified K562 cells, to produce large numbers of CD3⁻56⁺ NK cells with greater than 70% purity from peripheral blood mononuclear cells (PBMCs). CD3-depleted, CD56-enriched PBMCs can be cultured in the presence of EBV-LCL feeders and X-VIVO 20 medium supplemented with 10% heat inactivated human AB serum, 500 U mL⁻¹ IL-2 and 2 mM L-alanyl-L-glutamine to yield 490±260-fold expansion of NK cells over 21 days of culture, with a purity of 84.3±7.8% CD56⁺CD16⁺ cells (Berg et al. Cytotherapy, 11(3):341-55, 2009).

In some instances, NK cells can be expanded using a genetically modified feeder cell expansion system, as described, for example, in Yang et al. (Mol. Therapy 18:428-445, 2020). In such expansion methods, human primary NK cells can be expanded directly from PBMCs and cord blood (CB), as well as tumor tissue, using an irradiated, genetically engineered 721.221 cell line (a B cell line derived through mutagenesis that does not express dominant major histocompatibility complex (MHC) class I molecules or expresses a low amount of MHC class I molecules) that expresses membrane-bound interleukin 21 (IL-21) (221-mIL-21), as previous studies show the importance of IL-21 in NK expansion (Ojo et al. Sci. Rep. 9:14916, 2019). In combination with two recombinant cytokines (IL-15 and IL-2), primary NK cells can be expanded nearly 100,000-fold after 2 to 3 weeks of expansion.

Differentiation of NK Cells from Stem Cells

NK cells can be differentiated from stem cells by various methods known in the art. In some instances, NK cells can be differentiated from induced pluripotent stem cells (iPSCs), human embryonic stem cells (hESCs), mesenchymal stem cells (MSCs), or hematopoietic stem cells (HSCs). Protocols for the differentiation of NK cells from iPSCs and hESCs are described, for example, in Bock et al. J. Vis. Exp. (74):e50337, 2013; Knorr et al. Stem Cells Transl. Med. 2(4):274-83, 2013; Ni et al. Methods Mol. Biol. 1029:33-41, 2013; Zhu and Kaufman (Methods Mol. Biol. 2048:107-19, 2019). In order to differentiate iPSCs to CD34⁺CD45⁺ HPCs, embryonic bodies (EB) can be generated using different approaches, such as spinning of single cell iPSCs in round-shaped wells (spin EBs), culture on murine stroma cells, or direct induction of iPSC monolayer fragments in media with cytokines inducing differentiation towards the hematopoietic lineage. HPCs can be enriched by cell sorting or cell separation of CD34⁺ and/or CD45⁺ cells, and subsequently placed on murine feeder cells (e.g., AFT024, OP9, MS-5, EL08-1D2) in medium containing IL-3 (during the first week), IL-7, IL-15, SCF, IL-2, and Flt3L. NK-cells can also be differentiated without usage of xenogeneic stromal feeder cells, as described, e.g., by Knorr et al. Stem Cells Transl. Med. 2(4):274-83, 2013. CD3″CD56^{bright}CD16^{+/-} NK cells can be differentiated from hiPSC up to stage 4b (NKp80⁺) on OP9-DL1 stroma cells and are highly functional in terms of degranulation, cytokine production and cytotoxicity including antibody-dependent cellular cytotoxicity (ADCC). NK cell yield can be considerably increased through inactivation of feeder cells with mitomycin-C(MMC) without impacting on maturation or functional properties.

Additionally or in alternative, CD56⁺CD16⁺CD3″ NK cells can be differentiated from human iPSCs and NK-cell development can be characterized by surface expression of NK-lineage markers, as described, e.g., by Euchner et al. Front. Immunol. 12:640672, 2021. Hematopoietic priming of human iPSCs can result in CD34⁺CD45⁺ hematopoietic progenitor cells (HPC) that do not require enrichment for NK lymphocyte propagation. HPC can be further differentiated into NK cells on OP9-DL1 feeder cells resulting in high purity of CD56^{bright}CD16⁻ and CD56^{bright}CD16⁺ NK cells. The output of generated NK cells can be increased by inactivating OP9-DL1 feeder cells with MMC. CD7 expression can be detected from the first week of differentiation indicating priming towards the lymphoid lineage. CD56^{bright}CD16^{-/+} NK cells expressed high levels of DNAM-1, CD69, natural killer cell receptors NKG2A and NKG2D, and natural cytotoxicity receptors NKp46, NKp44, NKp30. Differentiation of NK cells up to stage 4b can be confirmed by assessing the expression of NKp80 on NK cells, and by a perforin⁺ and granzyme B⁺ phenotype. Differentiation of NK cells can also be confirmed by assessing killer cell immunoglobulin-like receptor KIR2DL2/DL3 and KIR3DL1 on NK cells.

In some instances, CD3⁻CD56⁺ NK cells can be differentiated from CD34⁺ hematopoietic progenitors cells (HPCs), as described, e.g., by Cichocki et al. Front Immunol, 10: 2078, 2019. NK cell development can occur along a continuum whereby common lymphocyte progenitors (CLPs) gradually downregulate CD34 and upregulate CD56. Acquisition of CD94 marks commitment to the CD56^{bright} stage, and CD56^{bright} NK cells subsequently differentiate into CD56^{di}m NK cells that upregulate CD16 and killer immunoglobulin-like receptors (KIR). Support for this linear model comes from analyses of cell populations in secondary lymphoid tissues and in vitro studies of NK cell development from HPCs.

CD3⁻CD56⁺ NK cells with cytotoxic function can be differentiated in vitro after long-term culture of CD34⁺ cells isolated from cord blood, bone marrow, fetal liver, thymus, or secondary lymphoid tissue with IL-2 or IL-15, as described, e.g., by Mrozek et al. Blood 87:2632-40, 1996; Jaleco et al. J. Immunol. 159:694-702, 1997; Sanchez et al. J. Exp. Med. 178:1857-66, 1993; and Freud et al. Immunity 22:295-304, 2005.

### 2. Stem Cells

In some embodiments, the immune cells of the present disclosure may be stem cells, such as induced pluripotent stem cells (iPSCs), mesenchymal stem cells (MSCs), or hematopoietic stem cells (HSCs). The pluripotent stem cells used herein may be induced pluripotent stem (iPS) cells. The induction of pluripotency was originally achieved by reprogramming of somatic cells via the introduction of transcription factors that are linked to pluripotency. The use of iPSCs circumvents most of the ethical and practical problems associated with large-scale clinical use of ES cells, and patients with iPSC-derived autologous transplants may not require lifelong immunosuppressive treatments to prevent graft rejection.

With the exception of germ cells, any cell can be used as a starting point for iPSCs. For example, cell types could be keratinocytes, fibroblasts, hematopoietic cells, mesenchymal cells, liver cells, or stomach cells. There is no limitation on the degree of cell differentiation or the age of an animal from which cells are collected. For example, undifferentiated progenitor cells (including somatic stem cells) and finally differentiated mature cells can be used as sources of somatic cells in the methods disclosed herein.

Somatic cells can be reprogrammed to produce iPS cells using methods known to one of skill in the art. One of skill in the art can readily produce iPS cells, see for example, U.S. Patent Appl. Publ. Nos. 2009/0246875, 2010/0210014, 2011/0104125, and 2012/0276636; U.S. Pat. Nos. 8,058,065, 8,129,187, 8,268,620, 8,546,140, 9,175,268, 8,741,648, and 8,691,574; and PCT Publication No. WO 2007/069666 A1, all of which are incorporated herein by reference. Generally, nuclear reprogramming factors are used to produce pluripotent stem cells from a somatic cell. In some embodiments, at least three, or at least four, of Klf4, c-Myc, Oct3/4, Sox2, Nanog, and Lin28 are utilized. In other embodiments, Oct3/4, Sox2, c-Myc and Klf4 or Oct3/4, Sox2, Nanog, and Lin28 are utilized. Mouse and human cDNA sequences of these nuclear reprogramming substances are available with reference to the NCBI accession numbers mentioned in WO2007/069666 and U.S. Pat. No. 8,183,038, which are incorporated herein by reference. Methods for introducing one or more reprogramming substances, or nucleic acids encoding these reprogramming substances, are known in the art, and disclosed for example, in U.S. Pat. Nos. 8,268,620, 8,691,574, 8,741,648, 8,546, 140, 8,900,871 and 8,071,369, all of which are incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used with the methods disclosed herein.

In certain embodiments, undefined conditions may be used; for example, pluripotent cells may be cultured on fibroblast feeder cells or a medium that has been exposed to fibroblast feeder cells in order to maintain the stem cells in an undifferentiated state. In some embodiments, the cell is cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR™ medium or E8™/Essential 8™ medium.

### 3. Genetically Engineered Antigen Receptors

The immune cells of the disclosure (e.g., autologous or allogeneic T cells (e.g., regulatory T cells, CD4⁺ T cells, CD8⁺ T cells, or gamma-delta T cells), NK cells, invariant NK cells, NKT cells, stem cells (e.g., MSCs or iPS cells) can be genetically engineered to express antigen receptors such as engineered CARs and/or TCRs. For example, the host cells (e.g, autologous or allogeneic NK cells) are modified to express a CAR having antigenic specificity for a cancer antigen. In particular embodiments, NK cells are engineered to express a CAR. The NK cells may be further engineered to express a TCR. Multiple CARs and/or TCRs, such as to different antigens, may be added to a single cell type, such as NK cells. Suitable methods of modification are known in the art (see, instance e.g., Sambrook et al. Molecular Cloning: A Laboratory Manual, 3rd ed., Cold Spring Harbor Press, Cold Spring Harbor, N.Y., 2001; and Ausubel et al., Current Protocols in Molecular Biology, Greene Publishing Associates and John Wiley & Sons, N Y, 1994).

In some embodiments, the cells comprise one or more nucleic acids introduced via genetic engineering that encode one or more antigen receptors, and genetically engineered products of such nucleic acids. In some embodiments, the nucleic acids are heterologous. In some embodiments, the nucleic acids are not naturally occurring, such as a nucleic acid not found in nature (e.g., chimeric).

In some embodiments, the CAR contains an extracellular antigen-recognition domain that specifically binds to an antigen (e.g., a tumor antigen or a pathogen antigen). In some embodiments, the antigen is a protein expressed on the surface of cells (e.g., cancerous cells).

Exemplary engineered antigen receptors, including CARs and recombinant TCRs, as well as methods for engineering and introducing the receptors into cells, include those described, for example, in PCT Publication Nos. WO 2000/14257, WO 2013/126726, WO 2012/129514, WO 2014/031687, WO 2013/166321, WO 2013/071154, and WO 2013/123061; U.S. Patent Application Publication Nos. US2002/131960, US2013/287748, and US2013/0149337; U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479,118; International Patent Application Publication No.: WO 2014/055668 A1 and European Patent Application Publication No. EP2537416.

### 4. Chimeric Antigen Receptors

In some aspects, the present disclosure provides a population of NK cells engineered to express a chimeric antigen receptor (CAR), and/or a polynucleotide encoding a CAR, wherein the CAR comprises (a) an extracellular domain comprising an antigen recognition domain that specifically binds human CD70; (b) a transmembrane domain; and (c) an intracellular domain. In some embodiments, the intracellular domain of the CAR comprises one or more (e.g., one, two, three, or more) co-stimulatory domains. In some embodiments, the intracellular domain of the CAR comprises one or more (e.g., one, two, three, or more) activation domains. In some embodiments, the CAR comprises a) an antigen recognition domain that specifically binds to human CD70, b) a hinge domain, c) a transmembrane domain, d) a costimulatory domain and e) an activation domain.

In some embodiments, the engineered antigen receptors include CARs, including activating or stimulatory CARs, co-stimulatory CARs (see, e.g., PCT Publ. No. WO 2014/055668), and/or inhibitory CARs (iCARs, see, e.g., Fedorov et al., Sci. Transl. Med. 5(215):215ra172, 2013).

A. Antigen Recognition Domains

In some embodiments, the antigen recognition domain of the CARs described herein may recognize an epitope comprising the shared space between one or more antigens. In some embodiments, the antigen recognition domain comprises complementary determining regions (CDRs) of a monoclonal antibody, variable regions of a monoclonal antibody, an scFv, a single domain antibody (e.g., a camelid single domain antibody), an antibody mimetic and/or antigen binding fragments thereof. In some embodiments, the specificity of the antigen recognition domain is derived from a protein or peptide (e.g., a ligand in a receptor-ligand pair) that specifically binds to another protein or peptide (e.g., a receptor in a receptor-ligand pair). In some embodiments, the antigen recognition domain comprises an aptamer, a T cell receptor (TCR)-like antibody, or a single chain TCR (scTCR). Almost any moiety that binds a given target (e.g., tumor associated antigen (TAA)) with high affinity can be used as an antigen recognition domain. The arrangement of the antigen recognition domain could be multimeric, such as a diabody or multimers. In some embodiments, the multimers can be formed by cross pairing of the variable portion of the light and heavy chains into a diabody.

In some embodiments, the antigen recognition domain of the CARs described herein comprises an antibody mimetic. The term “antibody mimetic” is intended to describe an organic compound that specifically binds a target sequence and has a structure distinct from a naturally-occurring antibody. Antibody mimetics may comprise a protein, a nucleic acid, or a small molecule. The target sequence to which an antibody mimetic of the disclosure specifically binds may be an antigen. Exemplary antibody mimetics include, but are not limited to, an affibody, an afflilin, an affimer, an affitin, an alphabody, an anticalin, an avimer (also known as avidity multimer), a DARPin (Designed Ankyrin Repeat Protein), a Fynomer, a Kunitz domain peptide, a monobody and a centyrin.

In some embodiments, CARs provided herein comprise a single chain variable fragments (scFv) derived from monoclonal antibodies specific for tumor associated antigen (e.g., CD70), with a hinge domain, a transmembrane domain, a costimulatory domain and a CD3z activation domain. Such molecules result in the transmission of a zeta signal in response to recognition by the scFv of its target. In some embodiments, the CARs provided herein are fusions of a receptor (e.g., CD27), with a hinge domain, a transmembrane domain, a costimulatory domain and a CD3z activation domain. Such molecules result in the transmission of a zeta signal in response to recognition by the receptor to its native ligand (e.g., CD70) expressed on the surface of a target cell.

Nucleic acids encoding any of the CARs described herein are also provided.

Nucleic acids encoding the CAR may be humanized. In some embodiments, the nucleic acid encoding a CAR provided herein is codon-optimized for expression in human cells. In some embodiments, the disclosure provides a full-length CAR cDNA or coding region.

In some embodiments, the antigen recognition domain of a CAR provided herein can comprise a CD27 polypeptide such as those described in WO 2012/058460, US 2018/0104337A1, US2013/0323214A1, EP 2632482, and EP 3372244, each of which is incorporated herein by reference in its entirety. Exemplary CD27 polypeptides that can be utilized as antigen recognition domains are reviewed in Starzer et al., (2020) ESMO Open, 4:e000629.

In some embodiments, the antigen recognition domain of a CAR provided herein comprises can comprise a fragment of the VH and VL chains of a single-chain variable fragment (scFv) that specifically bind CD70 or a CD27 polypeptide such as those described in U.S. Patent Appl. Publ. Nos. 2018/0230224, 2019/0233528, 2019/0233529; U.S. Pat. Nos. 8,124,738, 8,067,546, 8,562,987, 9,428,585, 9,701,752, 7,662,387, 8,535,678, 8,609,104, 8,663,642, 9,345,785, 7,641,903, 8,337,838, 8,647,624, 9,051,372, and 7,491,390; EP 1934261, EP 1871418, EP 1594542, EP 2100619, EP 2289559, EP 1799262 and EP 3583129 A1, each of which is incorporated herein by reference in its entirety. Exemplary CD70 antigen recognition domains include, but are not limited to, anti-CD70 antibodies reviewed in Starzer et al. (supra).

B. Exemplary Antigen Recognition Domains

In some embodiments, the antigen recognition domain of a CAR described herein binds (e.g., specifically binds) to CD70. The CD70-specific CAR, when expressed on the cell surface, redirects the specificity of NK cells to human CD70 (see, e.g., Accession Nos. NM 001252.5; NP 001243.1; NM 001330332.2; and NP 001317261.1).

i) Antigen Recognition Domains Comprising a CD27 Polypeptide

In some embodiments, the antigen recognition domain of a CAR provided herein comprises a CD27 polypeptide or an antigen binding fragment thereof (e.g., a fragment of CD27 that binds to CD70). Exemplary amino acid sequences of CD27 have been described (see, e.g., Accession Nos. NM_001242.4, NP_001233.1, XP_011519344.1, XM_011521042.3, XP_016875721.1, XM_017020232.1, XP_016875722, XM_017020233.2, XP_016875723, and XM_017020234.1). In some embodiments the antigen recognition domain of a CAR provided herein comprises a CD27 polypeptide sequence or an antigen binding fragment thereof as described in U.S. Patent Appl. Publ. No. 2018/0208671, incorporated herein by reference. In some embodiments, the antigen recognition domain of a CAR provided herein comprises or consists of a CD27 extracellular domain and a CD27 transmembrane domain. In some embodiments, the antigen recognition domain of a CAR provided herein comprises or consists of a CD27 signal peptide, a CD27 extracellular domain, and a CD27 transmembrane domain. In some embodiments, the antigen recognition domain of a CAR provided herein comprises or consists of a CD27 extracellular domain, and optionally comprises a signal peptide (e.g., a CD27 signal peptide).

Exemplary CD27 polypeptides of the disclosure comprises or consists of the amino acid sequence of SEQ ID NO: 6, 7, 8, 9, or 10.

In some embodiments, the antigen recognition domain comprises the CD27 signaling domain sequence comprising an amino acid sequence that is at least 90%, at least 91%, at least 92%, at least 93%, at least 94%, at least 95%, at least 96%, at least 97%, at least 98%, at least 99% or at least 100% identical to the amino acid sequence of SEQ ID NO: 7, 8, or 9. In some embodiments, the CD27 extracellular domain comprises a mutation. In some embodiments, the mutation in the CD27 extracellular domain reduces shedding of the CD27 extracellular domain.

ii) Antigen Recognition Domains Comprising an Anti-CD70 Antibody or Fragment Thereof

In some embodiments, the antigen recognition domain of a CAR provided herein comprises an antibody or an antigen-binding fragment thereof. In some embodiments, the antigen recognition domain of a CAR provided herein comprises a single chain antibody fragment (scFv) comprising a light chain variable domain (VL) and heavy chain variable domain (VH) of a monoclonal anti-CD70 antibody. Optionally, the VH and VL may be joined by a flexible linker, such as a glycine-serine linker or a Whitlow linker. In some embodiments, the scFv is humanized. In some embodiments, the antigen binding moiety may comprise VH and VL that are directionally linked, for example, from N to C terminus, VH-linker-VL or VL-linker-VH.

In some embodiments, the antigen recognition domain of a CAR provided herein comprises an scFv whose affinity for CD70 has been optimized to induce cytotoxicity of tumor cells that produce high levels of CD70 without inducing cytotoxicity of normal cell that express low or normal levels of CD70. Illustrative examples of such affinity tuning are provided in Caruso et al., (2015) Cancer Res, 75: 3505-18; and Liu et al., (2015) Cancer Res, 75: 3596-607.

In some embodiments, the antigen recognition domain of a CAR provided herein comprises a heavy chain variable domain comprising an amino acid sequence that is at least 90%, at least 91%, at least 92%, at least 93%, at least 94%, at least 95%, at least 96%, at least 97%, at least 98%, at least 99% or at least 100% identical to the amino acid sequence of any one of SEQ ID NOs:11, 21, 31, 41, 51, 61, 74, 78, 82, 92, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 694, 695, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, 958, 960, 962, 964, 966, 968, 970, 972, 974, 976, 978, 980, 982, 984, 986, 988, 990, 992, 994, 996, 998, 1000, 1002, 1004, 1006, 1008, 1010, 1012, 1014, 1016, 1018, 1020, 1022, 1024, 1026, 1028, 1030, 1032, 1034, 1036, 1038, 1040, 1042, 1044, 1046, 1048, 1050, 1052, 1054, 1056, 1058, 1060, 1062, 1064, 1066, 1068, 1070, 1072, 1074, 1076, 1078, 1080, 1082, 1084, 1086, 1088, 1090, 1092, 1094, 1096, 1098, 1100, 1102, 1104, 1106, 1108, 1110, 1112, 1114, 1116, 1118, 1120, 1122, 1124, 1126, 1128, 1130, 1132, 1134, 1136, 1138, 1140, 1142, 1144, 1146, 1148, 1150, 1152, 1154, 1156, 1158, 1160, 1162, 1164, 1166 or 1168. In some embodiments, the antigen recognition domain of a CAR provided herein comprises a light chain variable domain comprising an amino acid sequence that is at least 90%, at least 91%, at least 92%, at least 93%, at least 94%, at least 95%, at least 96%, at least 97%, at least 98%, at least 99% or at least 100% identical to the amino acid sequence of any one of SEQ ID NOs: 13, 23, 33, 43, 53, 63, 66, 69, 72, 76, 80, 84, 94, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, 957, 959, 961, 963, 965, 967, 969, 971, 973, 975, 977, 979, 981, 983, 985, 987, 989, 991, 993, 995, 997, 999, 1001, 1003, 1005, 1007, 1009, 1011, 1013, 1015, 1017, 1019, 1021, 1023, 1025, 1027, 1029, 1031, 1033, 1035, 1037, 1039, 1041, 1043, 1045, 1047, 1049, 1051, 1053, 1055, 1057, 1059, 1061, 1063, 1065, 1067, 1069, 1071, 1073, 1075, 1077, 1079, 1081, 1083, 1085, 1087, 1089, 1091, 1093, 1095, 1097, 1099, 1101, 1103, 1105, 1107, 1109, 1111, 1113, 1115, 1117, 1119, 1121, 1123, 1125, 1127, 1129, 1131, 1133, 1135, 1137, 1139, 1141, 1143, 1145, 1147, 1149, 1151, 1153, 1155, 1157, 1159, 1161, 1163, 1165, 1167 or 1169.

In some embodiments, the antigen recognition domain of a CAR provided herein comprises an amino acid sequence that is at least 90%, at least 91%, at least 92%, at least 93%, at least 94%, at least 95%, at least 96%, at least 97%, at least 98%, at least 99% or 100% identical to the amino acid sequence:

(SEQ ID NO: 2688)
QVQLVQSGAEVKKPGASVKVSCKASGYTFTNYGMNWVRQAPGQGLKWMGW
INTYTGEPTYADAFKGRVTMTRDTSISTAYMELSRLRSDDTAVYYCARDY
GDYGMDYWGQGTTVTVSSGGGGSGGGGSGGGGSGDIVMTQSPDSLAVSLG
ERATINCRASKSVSTSGYSFMHWYQQKPGQPPKLLIYLASNLESGVPDRF
SGSGSGTDFTLTISSLQAEDVAVYYCQHSREVPWTFGQGTKVEIK.

Exemplary anti-CD70 scFvs from which antigen recognition domains for use in a CAR described herein may be derived include, but are not limited to, 2H5 (MDX-1411 or MDX2H5), 10B4, 8B5, 18E7, 69A7 (MDX-1203 or MDX69A7), h1F6_VHE_VLA, h1F6_VHH_VLA, h1F6_VHJ_VLA, h1F6_VHM_VLA (SGN70 (based on vorzetuzumab)), h1F6_VHE_VLD, c1F6, 1F6-1, 2F2 and immunologically active and/or antigen-binding fragments thereof. Thus, in some embodiments, the antigen recognition domain of a CAR provided herein comprises a VH and VL derived from any one of the anti-CD70 antibodies 2H5, 10B4, 8B5, 18E7, 69A7, h1F6_VHE_VLA, h1F6_VHH_VLA, h1F6_VHJ_VLA, h1F6_VHM_VLA, h1F6_VHE_VLD, c1F6, 1F6-1, and 2F2.

In some embodiments, the antigen recognition domain of a CAR described herein comprises complementarity determining regions (CDRs) and/or a heavy chain variable domain (VH) and a light chain variable domain (VL) derived from the anti-CD70 antibody 2H5. The 2H5 antibody comprises a VH comprising the amino acid sequence of SEQ ID NO: 11, which comprises CDRH1, CDRH2, and CDRH3 comprising the amino acid sequence of SEQ ID NO: 15, 16, and 17, respectively; and a VL comprising the amino acid sequence of SEQ ID NO: 13, which comprises CDRL1, CDRL2, and CDRL3 comprising the amino acid sequence of SEQ ID NO: 18, 19, and 20, respectively.

In some embodiments, the antigen recognition domain of a CAR described herein comprises an scFv comprising a VH and a VL, wherein the VH comprises a CDRH1 of SEQ ID NO: 15, a CDRH2 of SEQ ID NO: 16, and a CDRH3 of SEQ ID NO: 17, and the VL comprises a CDRL1 of SEQ ID NO: 18, a CDRL2 of SEQ ID NO: 19, and a CDRL3 of SEQ ID NO: 20. In some embodiments, the antigen recognition domain of a CAR described herein comprising a VH and a VL, wherein the VH comprises the amino acid sequence of SEQ ID NO: 11, and the VL comprises the amino acid sequence of SEQ ID NO: 13.

In some embodiments, the antigen recognition domain of a CAR described herein comprises CDRs and/or a VH and a VL derived from the anti-CD70 antibody 10B4. The 10B4 antibody comprises a VH comprising the amino acid sequence of SEQ ID NO: 21, which comprises CDRH1, CDRH2, and CDRH3 comprising the amino acid sequence of SEQ ID NO: 25, 26, and 27, respectively; and a VL comprising the amino acid sequence of SEQ ID NO: 23, which comprises CDRL1, CDRL2, and CDRL3 comprising the amino acid sequence of SEQ ID NO: 28, 29, and 30, respectively.

In some embodiments, the antigen recognition domain of a CAR described herein comprises an scFv comprising a VH and a VL, wherein the VH comprises a CDRH1 of SEQ ID NO: 25, a CDRH2 of SEQ ID NO: 26, and a CDRH3 of SEQ ID NO: 27, and the VL comprises a CDRL1 of SEQ ID NO: 28, a CDRL2 of SEQ ID NO: 29, and a CDRL3 of SEQ ID NO: 30. In some embodiments, the antigen recognition domain of a CAR described herein comprises a VH and a VL, wherein the VH comprises the amino acid sequence of SEQ ID NO: 21, and the VL comprises the amino acid sequence of SEQ ID NO: 23.

In some embodiments, the antigen recognition domain of a CAR described herein comprises CDRs and/or a VH and a VL derived from the anti-CD70 antibody 8B5. The 8B5 antibody comprises a VH comprising the amino acid sequence of SEQ ID NO: 31, which comprises CDRH1, CDRH2, and CDRH3 comprising the amino acid sequence of SEQ ID NO: 35, 36, and 37, respectively; and a VL comprising the amino acid sequence of SEQ ID NO: 33, which comprises CDRL1, CDRL2, and CDRL3 comprising the amino acid sequence of SEQ ID NO: 38, 39, and 40, respectively.

In some embodiments, the antigen recognition domain of a CAR described herein comprises an scFv comprising a VH and a VL, wherein the VH comprises a CDRH1 of SEQ ID NO: 35, a CDRH2 of SEQ ID NO: 36, and a CDRH3 of SEQ ID NO: 37, and the VL comprises a CDRL1 of SEQ ID NO: 38, a CDRL2 of SEQ ID NO: 39, and a CDRL3 of SEQ ID NO: 40. In some embodiments, the antigen recognition domain of a CAR described herein comprises a VH and a VL, wherein the VH comprises the amino acid sequence of SEQ ID NO: 31, and the VL comprises the amino acid sequence of SEQ ID NO: 33.

In some embodiments, the antigen recognition domain of a CAR described herein comprises CDRs and/or a VH and a VL derived from the anti-CD70 antibody 18E7. The 18E7 antibody comprises a VH comprising the amino acid sequence of SEQ ID NO: 41, which comprises CDRH1, CDRH2, and CDRH3 comprising the amino acid sequence of SEQ ID NO: 45, 46, and 47, respectively; and a VL comprising the amino acid sequence of SEQ ID NO: 43, which comprises CDRL1, CDRL2, and CDRL3 comprising the amino acid sequence of SEQ ID NO: 48, 49, and 50, respectively.

In some embodiments, the antigen recognition domain of a CAR described herein comprises an scFv comprising a VH and a VL, wherein the VH comprises a CDRH1 of SEQ ID NO: 45, a CDRH2 of SEQ ID NO: 46, and a CDRH3 of SEQ ID NO: 47, and the VL comprises a CDRL1 of SEQ ID NO: 48, a CDRL2 of SEQ ID NO: 49, and a CDRL3 of SEQ ID NO: 50. In some embodiments, the antigen recognition domain of a CAR described herein comprises a VH and a VL, wherein the VH comprises the amino acid sequence of SEQ ID NO: 41, and the VL comprises the amino acid sequence of SEQ ID NO: 43.

In some embodiments, the antigen recognition domain of a CAR described herein comprises CDRs and/or a VH and a VL derived from the anti-CD70 antibody 69A7. The 69A7 antibody comprises a VH comprising the amino acid sequence of SEQ ID NO: 51, which comprises CDRH1, CDRH2, and CDRH3 comprising the amino acid sequence of SEQ ID NO: 55, 56, and 57, respectively; and a VL comprising the amino acid sequence of SEQ ID NO: 53, which comprises CDRL1, CDRL2, and CDRL3 comprising the amino acid sequence of SEQ ID NO: 58, 59, and 60, respectively.

In some embodiments, the antigen recognition domain of a CAR described herein comprises an scFv comprising a VH and a VL, wherein the VH comprises a CDRH1 of SEQ ID NO: 55, a CDRH2 of SEQ ID NO: 56, and a CDRH3 of SEQ ID NO: 57, and the VL comprises a CDRL1 of SEQ ID NO: 58, a CDRL2 of SEQ ID NO: 59, and a CDRL3 of SEQ ID NO: 60. In some embodiments, the antigen recognition domain of a CAR described herein comprises a VH and a VL, wherein the VH comprises the amino acid sequence of SEQ ID NO: 51, and the VL comprises the amino acid sequence of SEQ ID NO: 53.

In some embodiments, the antigen recognition domain of a CAR described herein comprises CDRs and/or a VH and a VL derived from the anti-CD70 antibody h1F6_VHE_VLA. The h1F6_VHE_VLA antibody comprises a VH comprising the amino acid sequence of SEQ ID NO: 61, and a VL comprising the amino acid sequence of SEQ ID NO: 63

In some embodiments, the antigen recognition domain of a CAR described herein comprises a VH and a VL, wherein the VH comprises the amino acid sequence of SEQ ID NO: 61, and the VL comprises the amino acid sequence of SEQ ID NO: 63.

In some embodiments, the antigen recognition domain of a CAR described herein comprises CDRs and/or a VH and a VL derived from the anti-CD70 antibody h1F6_VHH_VLA. The h1F6_VHH_VLA antibody comprises a VH comprising the amino acid sequence of SEQ ID NO: 693, and a VL comprising the amino acid sequence of SEQ ID NO: 66.

In some embodiments, the antigen recognition domain of a CAR described herein comprises a VH and a VL, wherein the VH comprises the amino acid sequence of SEQ ID NO: 693, and the VL comprises the amino acid sequence of SEQ ID NO: 66.

In some embodiments, the antigen recognition domain of a CAR described herein comprises CDRs and/or a VH and a VL derived from the anti-CD70 antibody h1F6_VHJ_VLA. The h1F6_VHJ_VLA antibody comprises a VH comprising the amino acid sequence of SEQ ID NO: 694, which comprises CDRH1, CDRH2, and CDRH3 comprising the amino acid sequence of SEQ ID NO: 2672, 2673, and 2674, respectively; and aVL comprising the amino acid sequence of SEQ ID NO: 69, which comprises CDRL1, CDRL2, and CDRL3 comprising the amino acid sequence of SEQ ID NO: 2675, 2676, and 2677, respectively.

In some embodiments, the antigen recognition domain of a CAR described herein comprises an scFv comprising a VH and a VL, wherein the VH comprises a CDRH1 of SEQ ID NO: 2672, a CDRH2 of SEQ ID NO: 2673, and a CDRH3 of SEQ ID NO: 2674, and the VL comprises a CDRL1 of SEQ ID NO: 2675, a CDRL2 of SEQ ID NO: 2676, and a CDRL3 of SEQ ID NO: 2677. In some embodiments, the antigen recognition domain of a CAR described herein comprises a VH and a VL, wherein the VH comprises the amino acid sequence of SEQ ID NO: 694, and the VL comprises the amino acid sequence of SEQ ID NO: 69.

In some embodiments, the antigen recognition domain of a CAR described herein comprises CDRs and/or a VH and VL derived from the anti-CD70 antibody h1F6_VHM_VLA. The h1F6_VHM_VLA antibody comprises a VH comprising the amino acid sequence of SEQ ID NO: 695, and a VL comprising the amino acid sequence of SEQ ID NO: 72.

In some embodiments, the antigen recognition domain of a CAR described herein comprises a VH and a VL, wherein the VH comprises the amino acid sequence of SEQ ID NO: 695, and the VL comprises the amino acid sequence of SEQ ID NO: 72.

In some embodiments, the antigen recognition domain of a CAR described herein comprises CDRs and/or a VH and a VL derived from the anti-CD70 antibody h1F6_VHD_VLA. The h1F6_VHD_VLA antibody comprises a VH comprising the amino acid sequence of SEQ ID NO: 74, and a VL comprising the amino acid sequence of SEQ ID NO: 76.

In some embodiments, the antigen recognition domain of a CAR described herein comprises a VH and a VL, wherein the VH comprises the amino acid sequence of SEQ ID NO: 74, and the VL comprises the amino acid sequence of SEQ ID NO: 76.

In some embodiments, the antigen recognition domain of a CAR described herein comprises CDRs and/or a VH and a VL derived from the anti-CD70 antibody c1F6. The c1F6 antibody comprises a VH comprising the amino acid sequence of SEQ ID NO: 78, and a VL comprising the amino acid sequence of SEQ ID NO: 80.

In some embodiments, the antigen recognition domain of a CAR described herein comprises a VH and a VL, wherein the VH comprises the amino acid sequence of SEQ ID NO: 78, and the VL comprises the amino acid sequence of SEQ ID NO: 80.

In some embodiments, the antigen recognition domain of a CAR described herein comprises CDRs and/or a VH and a VL derived from the anti-CD70 antibody 1F6_1. The 1F6_1 antibody comprises a VH comprising the amino acid sequence of SEQ ID NO: 82, which comprises CDRH1, CDRH2, and CDRH3 comprising the amino acid sequence of SEQ ID NO: 86, 87, and 88, respectively; and a VL comprising the amino acid sequence of SEQ ID NO: 84, which comprises CDRL1, CDRL2, and CDRL3 comprising the amino acid sequence of SEQ ID NO: 89, 90, and 91, respectively.

In some embodiments, the antigen recognition domain of a CAR described herein comprises an scFv comprising a VH and a VL, wherein the VH comprises a CDRH1 of SEQ ID NO: 86, a CDRH2 of SEQ ID NO: 87, and a CDRH3 of SEQ ID NO: 88, and the VL comprises a CDRL1 of SEQ ID NO: 89, a CDRL2 of SEQ ID NO: 90, and a CDRL3 of SEQ ID NO: 91. In some embodiments, the antigen recognition domain of a CAR described herein comprises a VH and a VL, wherein the VH comprises the amino acid sequence of SEQ ID NO: 82, and the VL comprises the amino acid sequence of SEQ ID NO: 84.

In some embodiments, the antigen recognition domain of a CAR described herein comprises CDRs and/or a VH and a VL derived from the anti-CD70 antibody 2F2. The 2F2 antibody comprises a VH comprising the amino acid sequence of SEQ ID NO: 92, which comprises CDRH1, CDRH2, and CDRH3 comprising the amino acid sequence of SEQ ID NO: 96, 97, and 98, respectively; and a VL comprising the amino acid sequence of SEQ ID NO: 94, which comprises CDRL1, CDRL2, and CDRL3 comprising the amino acid sequence of SEQ ID NO: 99, 100, and 101, respectively.

In some embodiments, the antigen recognition domain of a CAR described herein comprises an scFv comprising a VH and a VL, wherein the VH comprises a CDRH1 of SEQ ID NO: 96, a CDRH2 of SEQ ID NO: 97, and a CDRH3 of SEQ ID NO: 98, and the VL comprises a CDRL1 of SEQ ID NO: 99, a CDRL2 of SEQ ID NO: 100, and a CDRL3 of SEQ ID NO: 101. In some embodiments, the antigen recognition domain of a CAR described herein comprises a VH and a VL, wherein the VH comprises the amino acid sequence of SEQ ID NO: 92, and the VL comprises the amino acid sequence of SEQ ID NO: 94.

The antigen recognition domain of the CARs provided herein may include CDRs and/or VH and VL derived from an anti-CD70 antibody (or antigen binding fragment thereof). Exemplary anti-CD70 scFvs include but are not limited to 8G1, 1C8, 6E9, 31H1, 63B2, 40E3, 42C3, 45F11, 64F9, 72C2, 2F10, 4F11, 10H10, 17G6, 65E11, PO2B10, P07D03, P08A02, P08E02, P08F08, P08G02, P12B09, P12F02, P12G07, P13F04, P15D02, P16C05, 10A1, 10E2, 11A1, 11C1, 11D1, 11E1, 12A2, 12C4, 12C5, 12D3, 12D6, 12D7, 12F5, 12H4, 8C8, 8F7, 8F8, 9D8, 9E10, 9E5, 9F4, 9F8, 12C6, CD70-1, CD70-2, CD70-3, CD70-4, CD70-5, CD70-6, CD70-7, CD70-8, CD70-9, CD70-10, CD70-11, CD70-12, CD70-13, CD70-14, CD70-15, CD70-16, CD70-17, CD70-18, CD70-19, CD70-20, CD70-21, CD70-22, CD70-23, CD70-24, CD70-25, CD70-26, CD70-27, CD70-28, CD70-29, CD70-30, CD70-31, CD70-32, CD70-33, CD70-34, CD70-35, CD70-36, CD70-37, CD70-38, CD70-39, CD70-40, CD70-41, CD70-42, CD70-43, CD70-44, CD70-45, CD70-46, CD70-47, CD70-48, CD70-49, CD70-50, CD70-51, CD70-52, CD70-53, CD70-54, CD70-55, CD70-56, CD70-57, CD70-58, CD70-59, CD70-60, CD70-61, CD70-62, CD70-63, CD70-64, CD70-65, CD70-66, CD70-67, CD70-68, CD70-69, CD70-70, CD70-71, CD70-72, CD70-73, CD70-74, CD70-75, CD70-76, CD70-77, CD70-78, CD70-79, CD70-80, CD70-81, CD70-82, CD70-83, CD70-84, CD70-85, CD70-86, CD70-87, CD70-88, CD70-89, CD70-90, CD70-91, CD70-92, CD70-93, CD70-94, CD70-95, CD70-96, CD70-97, CD70-98, CD70-99, CD70-100, CD70-101, CD70-102, CD70-103, CD70-104, CD70-105, CD70-106, CD70-107, CD70-108, CD70-109, CD70-110, CD70-111, CD70-112, CD70-113, CD70-114, CD70-115, CD70-116, CD70-117, CD70-118, CD70-119, CD70-120, CD70-121, CD70-122, CD70-123, CD70-124, CD70-125, CD70-126, CD70-127, CD70-128, CD70-129, CD70-130, CD70-131, CD70-132, CD70-133, CD70-134, CD70-135, CD70-136, CD70-137, CD70-138, CD70-139, CD70-140, CD70-141, CD70-142, CD70-143, CD70-144, CD70-145, CD70-146, CD70-147, CD70-148, CD70-149, CD70-150, CD70-151, CD70-152, CD70-153, CD70-154, CD70-155, CD70-156, CD70-157, CD70-158, CD70-159, CD70-160, CD70-161, CD70-162, CD70-163, CD70-164, CD70-165, CD70-166, CD70-167, CD70-168, CD70-169, CD70-170, CD70-171, CD70-172, CD70-173, CD70-174, CD70-175, CD70-176, CD70-177, CD70-178, CD70-179, CD70-180, 1C2, 9D1, 8B12, 8C12, 9E1, 5F4, 5B2, 6D5, 4D2, 9A1, 9G2, 9B2, 24E3, 33D8, 24F2, 24B6, 19G10, 45B12, 45D9, 45F8, 45A12, 45B6, 57B6, 59D10, 27B3, 36A9, 53F1, 36D6, 53G1, 35G3, 53C1, 35F6, 36G2, 39D5, 42D12, 35C1, 41D12, 41H8, 35G2, 40F1, 53B1, 39C3, 53D1, 53H1, 53A2, cusatuzumab (ARGX-110), CTX-130, CTX-130, 4SCAR70, MDX-1411, SGN70, and immunologically active and/or antigen-binding fragments thereof. Anti-CD70 antibodies of the disclosure can comprise any one of the partial light chain sequences as listed in Table 1 and/or any one of partial heavy chain sequences as listed in Table 1. In some embodiments, the antigen recognition domain of a CAR described herein comprises an scFv comprising a VH and a VL, wherein the VH comprises the amino acid sequence of a VH from an anti-CD70 antibody listed in Table 1, and the VL comprises the amino acid sequence of the corresponding VL from the antibody listed in Table 1.

TABLE 1
Exemplary anti-CD70 antibodies - heavy
chain and light chain variable domains

| | heavy chain variable | light chain variable |
|---|---|---|
| Antibody ID | domain (VH) | domain (VL) |
| 31H1 | SEQ ID NO: 102 | SEQ ID NO: 103 |
| 63B2 | SEQ ID NO: 104 | SEQ ID NO: 105 |
| 40E3 | SEQ ID NO: 106 | SEQ ID NO: 107 |
| 42C3 | SEQ ID NO: 108 | SEQ ID NO: 109 |
| 45F11 | SEQ ID NO: 110 | SEQ ID NO: 111 |
| 64F9 | SEQ ID NO: 112 | SEQ ID NO: 113 |
| 72C2 | SEQ ID NO: 114 | SEQ ID NO: 115 |
| 2F10 | SEQ ID NO: 116 | SEQ ID NO: 117 |
| 4F11 | SEQ ID NO: 118 | SEQ ID NO: 119 |
| 10H10 | SEQ ID NO: 120 | SEQ ID NO: 121 |
| 17G6 | SEQ ID NO: 122 | SEQ ID NO: 123 |
| 65E11 | SEQ ID NO: 124 | SEQ ID NO: 125 |
| P02B10 | SEQ ID NO: 126 | SEQ ID NO: 127 |
| P07D03 | SEQ ID NO: 128 | SEQ ID NO: 129 |
| P08A02 | SEQ ID NO: 130 | SEQ ID NO: 131 |
| P08E02 | SEQ ID NO: 132 | SEQ ID NO: 133 |
| P08F08 | SEQ ID NO: 134 | SEQ ID NO: 135 |
| P08G02 | SEQ ID NO: 136 | SEQ ID NO: 137 |
| P12B09 | SEQ ID NO: 138 | SEQ ID NO: 139 |
| P12F02 | SEQ ID NO: 140 | SEQ ID NO: 141 |
| P12G07 | SEQ ID NO: 142 | SEQ ID NO: 143 |
| P13F04 | SEQ ID NO: 144 | SEQ ID NO: 145 |
| P15D02 | SEQ ID NO: 146 | SEQ ID NO: 147 |
| P16C05 | SEQ ID NO: 148 | SEQ ID NO: 149 |
| 10A1 | SEQ ID NO: 150 | SEQ ID NO: 151 |
| 10E2 | SEQ ID NO: 152 | SEQ ID NO: 153 |
| 11A1 | SEQ ID NO: 154 | SEQ ID NO: 155 |
| 11C1 | SEQ ID NO: 156 | SEQ ID NO: 157 |
| 11D1 | SEQ ID NO: 158 | SEQ ID NO: 159 |
| 11E1 | SEQ ID NO: 160 | SEQ ID NO: 161 |
| 12A2 | SEQ ID NO: 162 | SEQ ID NO: 163 |
| 12C4 | SEQ ID NO: 164 | SEQ ID NO: 165 |
| 12C5 | SEQ ID NO: 166 | SEQ ID NO: 167 |
| 12D3 | SEQ ID NO: 168 | SEQ ID NO: 169 |
| 12D6 | SEQ ID NO: 170 | SEQ ID NO: 171 |
| 12D7 | SEQ ID NO: 172 | SEQ ID NO: 173 |
| 12F5 | SEQ ID NO: 174 | SEQ ID NO: 175 |
| 12H4 | SEQ ID NO: 176 | SEQ ID NO: 177 |
| 8C8 | SEQ ID NO: 178 | SEQ ID NO: 179 |
| 8F7 | SEQ ID NO: 180 | SEQ ID NO: 181 |
| 8F8 | SEQ ID NO: 182 | SEQ ID NO: 183 |
| 9D8 | SEQ ID NO: 184 | SEQ ID NO: 185 |
| 9E10 | SEQ ID NO: 186 | SEQ ID NO: 187 |
| 9E5 | SEQ ID NO: 188 | SEQ ID NO: 189 |
| 9F4 | SEQ ID NO: 190 | SEQ ID NO: 191 |
| 9F8 | SEQ ID NO: 192 | SEQ ID NO: 193 |
| 12C6 | SEQ ID NO: 194 | SEQ ID NO: 195 |
| CD70-1 | SEQ ID NO: 712 | SEQ ID NO: 713 |
| CD70-2 | SEQ ID NO: 714 | SEQ ID NO: 715 |
| CD70-3 | SEQ ID NO: 716 | SEQ ID NO: 717 |
| CD70-4 | SEQ ID NO: 718 | SEQ ID NO: 719 |
| CD70-5 | SEQ ID NO: 720 | SEQ ID NO: 721 |
| CD70-6 | SEQ ID NO: 722 | SEQ ID NO: 723 |
| CD70-7 | SEQ ID NO: 724 | SEQ ID NO: 725 |
| CD70-8 | SEQ ID NO: 726 | SEQ ID NO: 727 |
| CD70-9 | SEQ ID NO: 728 | SEQ ID NO: 729 |
| CD70-10 | SEQ ID NO: 730 | SEQ ID NO: 731 |
| CD70-11 | SEQ ID NO: 732 | SEQ ID NO: 733 |
| CD70-12 | SEQ ID NO: 734 | SEQ ID NO: 735 |
| CD70-13 | SEQ ID NO: 736 | SEQ ID NO: 737 |
| CD70-14 | SEQ ID NO: 738 | SEQ ID NO: 739 |
| CD70-15 | SEQ ID NO: 740 | SEQ ID NO: 741 |
| CD70-16 | SEQ ID NO: 742 | SEQ ID NO: 743 |
| CD70-17 | SEQ ID NO: 744 | SEQ ID NO: 745 |
| CD70-18 | SEQ ID NO: 746 | SEQ ID NO: 747 |
| CD70-19 | SEQ ID NO: 748 | SEQ ID NO: 749 |
| CD70-20 | SEQ ID NO: 750 | SEQ ID NO: 751 |
| CD70-21 | SEQ ID NO: 752 | SEQ ID NO: 753 |
| CD70-22 | SEQ ID NO: 754 | SEQ ID NO: 755 |
| CD70-23 | SEQ ID NO: 756 | SEQ ID NO: 757 |
| CD70-24 | SEQ ID NO: 758 | SEQ ID NO: 759 |
| CD70-25 | SEQ ID NO: 760 | SEQ ID NO: 761 |
| CD70-26 | SEQ ID NO: 762 | SEQ ID NO: 763 |
| CD70-27 | SEQ ID NO: 764 | SEQ ID NO: 765 |
| CD70-28 | SEQ ID NO: 766 | SEQ ID NO: 767 |
| CD70-29 | SEQ ID NO: 768 | SEQ ID NO: 769 |
| CD70-30 | SEQ ID NO: 770 | SEQ ID NO: 771 |
| CD70-31 | SEQ ID NO: 772 | SEQ ID NO: 773 |
| CD70-32 | SEQ ID NO: 774 | SEQ ID NO: 775 |
| CD70-33 | SEQ ID NO: 776 | SEQ ID NO: 777 |
| CD70-34 | SEQ ID NO: 778 | SEQ ID NO: 779 |
| CD70-35 | SEQ ID NO: 780 | SEQ ID NO: 781 |
| CD70-36 | SEQ ID NO: 782 | SEQ ID NO: 783 |
| CD70-37 | SEQ ID NO: 784 | SEQ ID NO: 785 |
| CD70-38 | SEQ ID NO: 786 | SEQ ID NO: 787 |
| CD70-39 | SEQ ID NO: 788 | SEQ ID NO: 789 |
| CD70-40 | SEQ ID NO: 790 | SEQ ID NO: 791 |
| CD70-41 | SEQ ID NO: 792 | SEQ ID NO: 793 |
| CD70-42 | SEQ ID NO: 794 | SEQ ID NO: 795 |
| CD70-43 | SEQ ID NO: 796 | SEQ ID NO: 797 |
| CD70-44 | SEQ ID NO: 798 | SEQ ID NO: 799 |
| CD70-45 | SEQ ID NO: 800 | SEQ ID NO: 801 |
| CD70-46 | SEQ ID NO: 802 | SEQ ID NO: 803 |
| CD70-47 | SEQ ID NO: 804 | SEQ ID NO: 805 |
| CD70-48 | SEQ ID NO: 806 | SEQ ID NO: 807 |
| CD70-49 | SEQ ID NO: 808 | SEQ ID NO: 809 |
| CD70-50 | SEQ ID NO: 810 | SEQ ID NO: 811 |
| CD70-51 | SEQ ID NO: 812 | SEQ ID NO: 813 |
| CD70-52 | SEQ ID NO: 814 | SEQ ID NO: 815 |
| CD70-53 | SEQ ID NO: 816 | SEQ ID NO: 817 |
| CD70-54 | SEQ ID NO: 818 | SEQ ID NO: 819 |
| CD70-55 | SEQ ID NO: 820 | SEQ ID NO: 821 |
| CD70-56 | SEQ ID NO: 822 | SEQ ID NO: 823 |
| CD70-57 | SEQ ID NO: 824 | SEQ ID NO: 825 |
| CD70-58 | SEQ ID NO: 826 | SEQ ID NO: 827 |
| CD70-59 | SEQ ID NO: 828 | SEQ ID NO: 829 |
| CD70-60 | SEQ ID NO: 830 | SEQ ID NO: 831 |
| CD70-61 | SEQ ID NO: 832 | SEQ ID NO: 833 |
| CD70-62 | SEQ ID NO: 834 | SEQ ID NO: 835 |
| CD70-63 | SEQ ID NO: 836 | SEQ ID NO: 837 |
| CD70-64 | SEQ ID NO: 838 | SEQ ID NO: 839 |
| CD70-65 | SEQ ID NO: 840 | SEQ ID NO: 841 |
| CD70-66 | SEQ ID NO: 842 | SEQ ID NO: 843 |
| CD70-67 | SEQ ID NO: 844 | SEQ ID NO: 845 |
| CD70-68 | SEQ ID NO: 846 | SEQ ID NO: 847 |
| CD70-69 | SEQ ID NO: 848 | SEQ ID NO: 849 |
| CD70-70 | SEQ ID NO: 850 | SEQ ID NO: 851 |
| CD70-71 | SEQ ID NO: 852 | SEQ ID NO: 853 |
| CD70-72 | SEQ ID NO: 854 | SEQ ID NO: 855 |
| CD70-73 | SEQ ID NO: 856 | SEQ ID NO: 857 |
| CD70-74 | SEQ ID NO: 858 | SEQ ID NO: 859 |
| CD70-75 | SEQ ID NO: 860 | SEQ ID NO: 861 |
| CD70-76 | SEQ ID NO: 862 | SEQ ID NO: 863 |
| CD70-77 | SEQ ID NO: 864 | SEQ ID NO: 865 |
| CD70-78 | SEQ ID NO: 866 | SEQ ID NO: 867 |
| CD70-79 | SEQ ID NO: 868 | SEQ ID NO: 869 |
| CD70-80 | SEQ ID NO: 870 | SEQ ID NO: 871 |
| CD70-81 | SEQ ID NO: 872 | SEQ ID NO: 873 |
| CD70-82 | SEQ ID NO: 874 | SEQ ID NO: 875 |
| CD70-83 | SEQ ID NO: 876 | SEQ ID NO: 877 |
| CD70-84 | SEQ ID NO: 878 | SEQ ID NO: 879 |
| CD70-85 | SEQ ID NO: 880 | SEQ ID NO: 881 |
| CD70-86 | SEQ ID NO: 882 | SEQ ID NO: 883 |
| CD70-87 | SEQ ID NO: 884 | SEQ ID NO: 885 |
| CD70-88 | SEQ ID NO: 886 | SEQ ID NO: 887 |
| CD70-89 | SEQ ID NO: 888 | SEQ ID NO: 889 |
| CD70-90 | SEQ ID NO: 890 | SEQ ID NO: 891 |
| CD70-91 | SEQ ID NO: 892 | SEQ ID NO: 893 |
| CD70-92 | SEQ ID NO: 894 | SEQ ID NO: 895 |
| CD70-93 | SEQ ID NO: 896 | SEQ ID NO: 897 |
| CD70-94 | SEQ ID NO: 898 | SEQ ID NO: 899 |
| CD70-95 | SEQ ID NO: 900 | SEQ ID NO: 901 |
| CD70-96 | SEQ ID NO: 902 | SEQ ID NO: 903 |
| CD70-97 | SEQ ID NO: 904 | SEQ ID NO: 905 |
| CD70-98 | SEQ ID NO: 906 | SEQ ID NO: 907 |
| CD70-99 | SEQ ID NO: 908 | SEQ ID NO: 909 |
| CD70-100 | SEQ ID NO: 910 | SEQ ID NO: 911 |
| CD70-101 | SEQ ID NO: 912 | SEQ ID NO: 913 |
| CD70-102 | SEQ ID NO: 914 | SEQ ID NO: 915 |
| CD70-103 | SEQ ID NO: 916 | SEQ ID NO: 917 |
| CD70-104 | SEQ ID NO: 918 | SEQ ID NO: 919 |
| CD70-105 | SEQ ID NO: 920 | SEQ ID NO: 921 |
| CD70-106 | SEQ ID NO: 922 | SEQ ID NO: 923 |
| CD70-107 | SEQ ID NO: 924 | SEQ ID NO: 925 |
| CD70-108 | SEQ ID NO: 926 | SEQ ID NO: 927 |
| CD70-109 | SEQ ID NO: 928 | SEQ ID NO: 929 |
| CD70-110 | SEQ ID NO: 930 | SEQ ID NO: 931 |
| CD70-111 | SEQ ID NO: 932 | SEQ ID NO: 933 |
| CD70-112 | SEQ ID NO: 934 | SEQ ID NO: 935 |
| CD70-113 | SEQ ID NO: 936 | SEQ ID NO: 937 |
| CD70-114 | SEQ ID NO: 938 | SEQ ID NO: 939 |
| CD70-115 | SEQ ID NO: 940 | SEQ ID NO: 941 |
| CD70-116 | SEQ ID NO: 942 | SEQ ID NO: 943 |
| CD70-117 | SEQ ID NO: 944 | SEQ ID NO: 945 |
| CD70-118 | SEQ ID NO: 946 | SEQ ID NO: 947 |
| CD70-119 | SEQ ID NO: 948 | SEQ ID NO: 949 |
| CD70-120 | SEQ ID NO: 950 | SEQ ID NO: 951 |
| CD70-121 | SEQ ID NO: 952 | SEQ ID NO: 953 |
| CD70-122 | SEQ ID NO: 954 | SEQ ID NO: 955 |
| CD70-123 | SEQ ID NO: 956 | SEQ ID NO: 957 |
| CD70-124 | SEQ ID NO: 958 | SEQ ID NO: 959 |
| CD70-125 | SEQ ID NO: 960 | SEQ ID NO: 961 |
| CD70-126 | SEQ ID NO: 962 | SEQ ID NO: 963 |
| CD70-127 | SEQ ID NO: 964 | SEQ ID NO: 965 |
| CD70-128 | SEQ ID NO: 966 | SEQ ID NO: 967 |
| CD70-129 | SEQ ID NO: 968 | SEQ ID NO: 969 |
| CD70-130 | SEQ ID NO: 970 | SEQ ID NO: 971 |
| CD70-131 | SEQ ID NO: 972 | SEQ ID NO: 973 |
| CD70-132 | SEQ ID NO: 974 | SEQ ID NO: 975 |
| CD70-133 | SEQ ID NO: 976 | SEQ ID NO: 977 |
| CD70-134 | SEQ ID NO: 978 | SEQ ID NO: 979 |
| CD70-135 | SEQ ID NO: 980 | SEQ ID NO: 981 |
| CD70-136 | SEQ ID NO: 982 | SEQ ID NO: 983 |
| CD70-137 | SEQ ID NO: 984 | SEQ ID NO: 985 |
| CD70-138 | SEQ ID NO: 986 | SEQ ID NO: 987 |
| CD70-139 | SEQ ID NO: 988 | SEQ ID NO: 989 |
| CD70-140 | SEQ ID NO: 990 | SEQ ID NO: 991 |
| CD70-141 | SEQ ID NO: 992 | SEQ ID NO: 993 |
| CD70-142 | SEQ ID NO: 994 | SEQ ID NO: 995 |
| CD70-143 | SEQ ID NO: 996 | SEQ ID NO: 997 |
| CD70-144 | SEQ ID NO: 998 | SEQ ID NO: 999 |
| CD70-145 | SEQ ID NO: 1000 | SEQ ID NO: 1001 |
| CD70-146 | SEQ ID NO: 1002 | SEQ ID NO: 1003 |
| CD70-147 | SEQ ID NO: 1004 | SEQ ID NO: 1005 |
| CD70-148 | SEQ ID NO: 1006 | SEQ ID NO: 1007 |
| CD70-149 | SEQ ID NO: 1008 | SEQ ID NO: 1009 |
| CD70-150 | SEQ ID NO: 1010 | SEQ ID NO: 1011 |
| CD70-151 | SEQ ID NO: 1012 | SEQ ID NO: 1013 |
| CD70-152 | SEQ ID NO: 1014 | SEQ ID NO: 1015 |
| CD70-153 | SEQ ID NO: 1016 | SEQ ID NO: 1017 |
| CD70-154 | SEQ ID NO: 1018 | SEQ ID NO: 1019 |
| CD70-155 | SEQ ID NO: 1020 | SEQ ID NO: 1021 |
| CD70-156 | SEQ ID NO: 1022 | SEQ ID NO: 1023 |
| CD70-157 | SEQ ID NO: 1024 | SEQ ID NO: 1025 |
| CD70-158 | SEQ ID NO: 1026 | SEQ ID NO: 1027 |
| CD70-159 | SEQ ID NO: 1028 | SEQ ID NO: 1029 |
| CD70-160 | SEQ ID NO: 1030 | SEQ ID NO: 1031 |
| CD70-161 | SEQ ID NO: 1032 | SEQ ID NO: 1033 |
| CD70-162 | SEQ ID NO: 1034 | SEQ ID NO: 1035 |
| CD70-163 | SEQ ID NO: 1036 | SEQ ID NO: 1037 |
| CD70-164 | SEQ ID NO: 1038 | SEQ ID NO: 1039 |
| CD70-165 | SEQ ID NO: 1040 | SEQ ID NO: 1041 |
| CD70-166 | SEQ ID NO: 1042 | SEQ ID NO: 1043 |
| CD70-167 | SEQ ID NO: 1044 | SEQ ID NO: 1045 |
| CD70-168 | SEQ ID NO: 1046 | SEQ ID NO: 1047 |
| CD70-169 | SEQ ID NO: 1048 | SEQ ID NO: 1049 |
| CD70-170 | SEQ ID NO: 1050 | SEQ ID NO: 1051 |
| CD70-171 | SEQ ID NO: 1052 | SEQ ID NO: 1053 |
| CD70-172 | SEQ ID NO: 1054 | SEQ ID NO: 1055 |
| CD70-173 | SEQ ID NO: 1056 | SEQ ID NO: 1057 |
| CD70-174 | SEQ ID NO: 1058 | SEQ ID NO: 1059 |
| CD70-175 | SEQ ID NO: 1060 | SEQ ID NO: 1061 |
| CD70-176 | SEQ ID NO: 1062 | SEQ ID NO: 1063 |
| CD70-177 | SEQ ID NO: 1064 | SEQ ID NO: 1065 |
| CD70-178 | SEQ ID NO: 1066 | SEQ ID NO: 1067 |
| CD70-179 | SEQ ID NO: 1068 | SEQ ID NO: 1069 |
| CD70-180 | SEQ ID NO: 1070 | SEQ ID NO: 1071 |
| 1C2 | SEQ ID NO: 1072 | SEQ ID NO: 1073 |
| 9D1 | SEQ ID NO: 1074 | SEQ ID NO: 1075 |
| 8B12 | SEQ ID NO: 1076 | SEQ ID NO: 1077 |
| 8C12 | SEQ ID NO: 1078 | SEQ ID NO: 1079 |
| 9E1 | SEQ ID NO: 1080 | SEQ ID NO: 1081 |
| 5F4 | SEQ ID NO: 1082 | SEQ ID NO: 1083 |
| 5B2 | SEQ ID NO: 1084 | SEQ ID NO: 1085 |
| 6D5 | SEQ ID NO: 1086 | SEQ ID NO: 1087 |
| 4D2 | SEQ ID NO: 1088 | SEQ ID NO: 1089 |
| 9A1 | SEQ ID NO: 1090 | SEQ ID NO: 1091 |
| 9G2 | SEQ ID NO: 1092 | SEQ ID NO: 1093 |
| 9B2 | SEQ ID NO: 1094 | SEQ ID NO: 1095 |
| 24E3 | SEQ ID NO: 1096 | SEQ ID NO: 1097 |
| 33D8 | SEQ ID NO: 1098 | SEQ ID NO: 1099 |
| 24F2 | SEQ ID NO: 1100 | SEQ ID NO: 1101 |
| 24B6 | SEQ ID NO: 1102 | SEQ ID NO: 1103 |
| 19G10 | SEQ ID NO: 1104 | SEQ ID NO: 1105 |
| 45B12 | SEQ ID NO: 1106 | SEQ ID NO: 1107 |
| 45D9 | SEQ ID NO: 1108 | SEQ ID NO: 1109 |
| 45F8 | SEQ ID NO: 1110 | SEQ ID NO: 1111 |
| 45A12 | SEQ ID NO: 1112 | SEQ ID NO: 1113 |
| 45B6 | SEQ ID NO: 1114 | SEQ ID NO: 1115 |
| 57B6 | SEQ ID NO: 1116 | SEQ ID NO: 1117 |
| 59D10 | SEQ ID NO: 1118 | SEQ ID NO: 1119 |
| 27B3 | SEQ ID NO: 1120 | SEQ ID NO: 1121 |
| 36A9 | SEQ ID NO: 1122 | SEQ ID NO: 1123 |
| 53F1 | SEQ ID NO: 1124 | SEQ ID NO: 1125 |
| 36D6 | SEQ ID NO: 1126 | SEQ ID NO: 1127 |
| 53G1 | SEQ ID NO: 1128 | SEQ ID NO: 1129 |
| 35G3 | SEQ ID NO: 1130 | SEQ ID NO: 1131 |
| 53C1 | SEQ ID NO: 1132 | SEQ ID NO: 1133 |
| 35F6 | SEQ ID NO: 1134 | SEQ ID NO: 1135 |
| 36G2 | SEQ ID NO: 1136 | SEQ ID NO: 1137 |
| 39D5 | SEQ ID NO: 1138 | SEQ ID NO: 1139 |
| 42D12 | SEQ ID NO: 1140 | SEQ ID NO: 1141 |
| 35C1 | SEQ ID NO: 1142 | SEQ ID NO: 1143 |
| 41D12 | SEQ ID NO: 1144 | SEQ ID NO: 1145 |
| 41H8 | SEQ ID NO: 1146 | SEQ ID NO: 1147 |
| 35G2 | SEQ ID NO: 1148 | SEQ ID NO: 1149 |
| 40F1 | SEQ ID NO: 1150 | SEQ ID NO: 1151 |
| 53B1 | SEQ ID NO: 1152 | SEQ ID NO: 1153 |
| 39C3 | SEQ ID NO: 1154 | SEQ ID NO: 1155 |
| 53D1 | SEQ ID NO: 1156 | SEQ ID NO: 1157 |
| 53H1 | SEQ ID NO: 1158 | SEQ ID NO: 1159 |
| 53A2 | SEQ ID NO: 1160 | SEQ ID NO: 1161 |
| ARGX-110 | SEQ ID NO: 1162 | SEQ ID NO: 1163 |
| CTX-130 | SEQ ID NO: 1164 | SEQ ID NO: 1165 |
| CTX-130 | SEQ ID NO: 1166 | SEQ ID NO: 1167 |
| 4SCAR70 | SEQ ID NO: 1168 | SEQ ID NO: 1169 |

In some embodiments, the antigen recognition domain of a CAR described herein comprises an scFv comprising a VH and a VL, wherein the VH comprises a CDRH1, a CDRH2, and a CDRH3 each comprising the amino acid sequence of a CDRH1, a CDRH2, and a CDRH3 of an anti-CD70 antibody as provided in Table 2, and wherein and the VL comprises a CDRL1, a CDRL2, and a CDRL3 each comprising the amino acid sequence of a CDRL1, a CDRL2, and a CDRL3 of the same anti-CD70 antibody as provided in Table 3. Determination of CDR regions is well within the skill of the art. It is understood that in some embodiments, CDRs can be a combination of the Kabat and Chothia CDR (also termed “combined CRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof.

TABLE 2
Exemplary heavy chain complementarity determining
regions of anti-CD70 antibodies

| Antibody ID | | CDRH1 | CDRH2 | CDRH3 |
|---|---|---|---|---|
| 31H1 | Kabat | SEQ ID NO: 196 | SEQ ID NO: 197 | SEQ ID NO: 198 |
| | Chothia | SEQ ID NO: 199 | SEQ ID NO: 200 | |
| | Extended | SEQ ID NO: 201 | | |
| 63B2 | Kabat | SEQ ID NO: 202 | SEQ ID NO: 203 | SEQ ID NO: 204 |
| | Chothia | SEQ ID NO: 205 | SEQ ID NO: 206 | |
| | Extended | SEQ ID NO: 207 | | |
| 40E3 | Kabat | SEQ ID NO: 208 | SEQ ID NO: 209 | SEQ ID NO: 210 |
| | Chothia | SEQ ID NO: 211 | SEQ ID NO: 212 | |
| | Extended | SEQ ID NO: 213 | | |
| 42C3 | Kabat | SEQ ID NO: 214 | SEQ ID NO: 215 | SEQ ID NO: 216 |
| | Chothia | SEQ ID NO: 217 | SEQ ID NO: 218 | |
| | Extended | SEQ ID NO: 219 | | |
| 45F11 | Kabat | SEQ ID NO: 220 | SEQ ID NO: 221 | SEQ ID NO: 222 |
| | Chothia | SEQ ID NO: 223 | SEQ ID NO: 224 | |
| | Extended | SEQ ID NO: 225 | | |
| 64F9 | Kabat | SEQ ID NO: 226 | SEQ ID NO: 227 | SEQ ID NO: 228 |
| | Chothia | SEQ ID NO: 229 | SEQ ID NO: 230 | |
| | Extended | SEQ ID NO: 231 | | |
| 72C2 | Kabat | SEQ ID NO: 232 | SEQ ID NO: 233 | SEQ ID NO: 234 |
| | Chothia | SEQ ID NO: 235 | SEQ ID NO: 236 | |
| | Extended | SEQ ID NO: 237 | | |
| 2F10 | Kabat | SEQ ID NO: 238 | SEQ ID NO: 239 | SEQ ID NO: 240 |
| | Chothia | SEQ ID NO: 241 | SEQ ID NO: 242 | |
| | Extended | SEQ ID NO: 243 | | |
| 4F11 | Kabat | SEQ ID NO: 244 | SEQ ID NO: 245 | SEQ ID NO: 246 |
| | Chothia | SEQ ID NO: 247 | SEQ ID NO: 248 | |
| | Extended | SEQ ID NO: 249 | | |
| 10H10 | Kabat | SEQ ID NO: 250 | SEQ ID NO: 251 | SEQ ID NO: 252 |
| | Chothia | SEQ ID NO: 253 | SEQ ID NO: 254 | |
| | Extended | SEQ ID NO: 255 | | |
| 17G6 | Kabat | SEQ ID NO: 256 | SEQ ID NO: 257 | SEQ ID NO: 258 |
| | Chothia | SEQ ID NO: 259 | SEQ ID NO: 260 | |
| | Extended | SEQ ID NO: 261 | | |
| 65E11 | Kabat | SEQ ID NO: 262 | SEQ ID NO: 263 | SEQ ID NO: 264 |
| | Chothia | SEQ ID NO: 265 | SEQ ID NO: 266 | |
| | Extended | SEQ ID NO: 267 | | |
| P02B10 | Kabat | SEQ ID NO: 268 | SEQ ID NO: 269 | SEQ ID NO: 270 |
| | Chothia | SEQ ID NO: 271 | SEQ ID NO: 272 | |
| | Extended | SEQ ID NO: 273 | | |
| P07D03 | Kabat | SEQ ID NO: 274 | SEQ ID NO: 275 | SEQ ID NO: 276 |
| | Chothia | SEQ ID NO: 277 | SEQ ID NO: 278 | |
| | Extended | SEQ ID NO: 279 | | |
| P08A02 | Kabat | SEQ ID NO: 280 | SEQ ID NO: 281 | SEQ ID NO: 282 |
| | Chothia | SEQ ID NO: 283 | SEQ ID NO: 284 | |
| | Extended | SEQ ID NO: 285 | | |
| P08E02 | Kabat | SEQ ID NO: 286 | SEQ ID NO: 287 | SEQ ID NO: 288 |
| | Chothia | SEQ ID NO: 289 | SEQ ID NO: 290 | |
| | Extended | SEQ ID NO: 291 | | |
| P08F08 | Kabat | SEQ ID NO: 292 | SEQ ID NO: 293 | SEQ ID NO: 294 |
| | Chothia | SEQ ID NO: 295 | SEQ ID NO: 296 | |
| | Extended | SEQ ID NO: 297 | | |
| P08G02 | Kabat | SEQ ID NO: 298 | SEQ ID NO: 299 | SEQ ID NO: 300 |
| | Chothia | SEQ ID NO: 301 | SEQ ID NO: 302 | |
| | Extended | SEQ ID NO: 303 | | |
| P12B09 | Kabat | SEQ ID NO: 304 | SEQ ID NO: 305 | SEQ ID NO: 306 |
| | Chothia | SEQ ID NO: 307 | SEQ ID NO: 308 | |
| | Extended | SEQ ID NO: 309 | | |
| P12F02 | Kabat | SEQ ID NO: 310 | SEQ ID NO: 311 | SEQ ID NO: 312 |
| | Chothia | SEQ ID NO: 313 | SEQ ID NO: 314 | |
| | Extended | SEQ ID NO: 315 | | |
| P12G07 | Kabat | SEQ ID NO: 316 | SEQ ID NO: 317 | SEQ ID NO: 318 |
| | Chothia | SEQ ID NO: 319 | SEQ ID NO: 320 | |
| | Extended | SEQ ID NO: 321 | | |
| P13F04 | Kabat | SEQ ID NO: 322 | SEQ ID NO: 323 | SEQ ID NO: 324 |
| | Chothia | SEQ ID NO: 325 | SEQ ID NO: 326 | |
| | Extended | SEQ ID NO: 327 | | |
| P15D02 | Kabat | SEQ ID NO: 328 | SEQ ID NO: 329 | SEQ ID NO: 330 |
| | Chothia | SEQ ID NO: 331 | SEQ ID NO: 332 | |
| | Extended | SEQ ID NO: 333 | | |
| P16C05 | Kabat | SEQ ID NO: 334 | SEQ ID NO: 335 | SEQ ID NO: 336 |
| | Chothia | SEQ ID NO: 337 | SEQ ID NO: 338 | |
| | Extended | SEQ ID NO: 339 | | |
| 10A1 | Kabat | SEQ ID NO: 340 | SEQ ID NO: 341 | SEQ ID NO: 342 |
| | Chothia | SEQ ID NO: 343 | SEQ ID NO: 344 | |
| | Extended | SEQ ID NO: 345 | | |
| 10E2 | Kabat | SEQ ID NO: 346 | SEQ ID NO: 347 | SEQ ID NO: 348 |
| | Chothia | SEQ ID NO: 349 | SEQ ID NO: 350 | |
| | Extended | SEQ ID NO: 351 | | |
| 11A1 | Kabat | SEQ ID NO: 352 | SEQ ID NO: 353 | SEQ ID NO: 354 |
| | Chothia | SEQ ID NO: 355 | SEQ ID NO: 356 | |
| | Extended | SEQ ID NO: 357 | | |
| 11C1 | Kabat | SEQ ID NO: 358 | SEQ ID NO: 359 | SEQ ID NO: 360 |
| | Chothia | SEQ ID NO: 361 | SEQ ID NO: 362 | |
| | Extended | SEQ ID NO: 363 | | |
| 11D1 | Kabat | SEQ ID NO: 364 | SEQ ID NO: 365 | SEQ ID NO: 366 |
| | Chothia | SEQ ID NO: 367 | SEQ ID NO: 368 | |
| | Extended | SEQ ID NO: 369 | | |
| 11E1 | Kabat | SEQ ID NO: 370 | SEQ ID NO: 371 | SEQ ID NO: 372 |
| | Chothia | SEQ ID NO: 373 | SEQ ID NO: 374 | |
| | Extended | SEQ ID NO: 375 | | |
| 12A2 | Kabat | SEQ ID NO: 376 | SEQ ID NO: 377 | SEQ ID NO: 378 |
| | Chothia | SEQ ID NO: 379 | SEQ ID NO: 380 | |
| | Extended | SEQ ID NO: 381 | | |
| 12C4 | Kabat | SEQ ID NO: 382 | SEQ ID NO: 383 | SEQ ID NO: 384 |
| | Chothia | SEQ ID NO: 385 | SEQ ID NO: 386 | |
| | Extended | SEQ ID NO: 387 | | |
| 12C5 | Kabat | SEQ ID NO: 388 | SEQ ID NO: 389 | SEQ ID NO: 390 |
| | Chothia | SEQ ID NO: 391 | SEQ ID NO: 392 | |
| | Extended | SEQ ID NO: 393 | | |
| 12D3 | Kabat | SEQ ID NO: 394 | SEQ ID NO: 395 | SEQ ID NO: 396 |
| | Chothia | SEQ ID NO: 397 | SEQ ID NO: 398 | |
| | Extended | SEQ ID NO: 399 | | |
| 12D6 | Kabat | SEQ ID NO: 400 | SEQ ID NO: 401 | SEQ ID NO: 402 |
| | Chothia | SEQ ID NO: 403 | SEQ ID NO: 404 | |
| | Extended | SEQ ID NO: 405 | | |
| 12D7 | Kabat | SEQ ID NO: 406 | SEQ ID NO: 407 | SEQ ID NO: 408 |
| | Chothia | SEQ ID NO: 409 | SEQ ID NO: 410 | |
| | Extended | SEQ ID NO: 411 | | |
| 12F5 | Kabat | SEQ ID NO: 412 | SEQ ID NO: 413 | SEQ ID NO: 414 |
| | Chothia | SEQ ID NO: 415 | SEQ ID NO: 416 | |
| | Extended | SEQ ID NO: 417 | | |
| 12H4 | Kabat | SEQ ID NO: 418 | SEQ ID NO: 419 | SEQ ID NO: 420 |
| | Chothia | SEQ ID NO: 421 | SEQ ID NO: 422 | |
| | Extended | SEQ ID NO: 423 | | |
| 8C8 | Kabat | SEQ ID NO: 424 | SEQ ID NO: 425 | SEQ ID NO: 426 |
| | Chothia | SEQ ID NO: 427 | SEQ ID NO: 428 | |
| | Extended | SEQ ID NO: 429 | | |
| 8F7 | Kabat | SEQ ID NO: 430 | SEQ ID NO: 431 | SEQ ID NO: 432 |
| | Chothia | SEQ ID NO: 433 | SEQ ID NO: 434 | |
| | Extended | SEQ ID NO: 435 | | |
| 8F8 | Kabat | SEQ ID NO: 436 | SEQ ID NO: 437 | SEQ ID NO: 438 |
| | Chothia | SEQ ID NO: 439 | SEQ ID NO: 440 | |
| | Extended | SEQ ID NO: 441 | | |
| 9D8 | Kabat | SEQ ID NO: 442 | SEQ ID NO: 443 | SEQ ID NO: 444 |
| | Chothia | SEQ ID NO: 445 | SEQ ID NO: 446 | |
| | Extended | SEQ ID NO: 447 | | |
| 9E10 | Kabat | SEQ ID NO: 448 | SEQ ID NO: 449 | SEQ ID NO: 450 |
| | Chothia | SEQ ID NO: 451 | SEQ ID NO: 452 | |
| | Extended | SEQ ID NO: 453 | | |
| 9E5 | Kabat | SEQ ID NO: 454 | SEQ ID NO: 455 | SEQ ID NO: 456 |
| | Chothia | SEQ ID NO: 457 | SEQ ID NO: 458 | |
| | Extended | SEQ ID NO: 459 | | |
| 9F4 | Kabat | SEQ ID NO: 460 | SEQ ID NO: 461 | SEQ ID NO: 462 |
| | Chothia | SEQ ID NO: 463 | SEQ ID NO: 464 | |
| | Extended | SEQ ID NO: 465 | | |
| 9F8 | Kabat | SEQ ID NO: 466 | SEQ ID NO: 467 | SEQ ID NO: 468 |
| | Chothia | SEQ ID NO: 469 | SEQ ID NO: 470 | |
| | Extended | SEQ ID NO: 471 | | |
| 12C6 | Kabat | SEQ ID NO: 472 | SEQ ID NO: 473 | SEQ ID NO: 474 |
| | Chothia | SEQ ID NO: 475 | SEQ ID NO: 476 | |
| | Extended | SEQ ID NO: 477 | | |
| CD70-1 | Kabat | SEQ ID NO: 1170 | SEQ ID NO: 1171 | SEQ ID NO: 1172 |
| CD70-2 | Kabat | SEQ ID NO: 1173 | SEQ ID NO: 1174 | SEQ ID NO: 1175 |
| CD70-3 | Kabat | SEQ ID NO: 1176 | SEQ ID NO: 1177 | SEQ ID NO: 1178 |
| CD70-4 | Kabat | SEQ ID NO: 1179 | SEQ ID NO: 1180 | SEQ ID NO: 1181 |
| CD70-5 | Kabat | SEQ ID NO: 1182 | SEQ ID NO: 1183 | SEQ ID NO: 1184 |
| CD70-6 | Kabat | SEQ ID NO: 1185 | SEQ ID NO: 1186 | SEQ ID NO: 1187 |
| CD70-7 | Kabat | SEQ ID NO: 1188 | SEQ ID NO: 1189 | SEQ ID NO: 1190 |
| CD70-8 | Kabat | SEQ ID NO: 1191 | SEQ ID NO: 1192 | SEQ ID NO: 1193 |
| CD70-9 | Kabat | SEQ ID NO: 1194 | SEQ ID NO: 1195 | SEQ ID NO: 1196 |
| CD70-10 | Kabat | SEQ ID NO: 1197 | SEQ ID NO: 1198 | SEQ ID NO: 1199 |
| CD70-11 | Kabat | SEQ ID NO: 1200 | SEQ ID NO: 1201 | SEQ ID NO: 1202 |
| CD70-12 | Kabat | SEQ ID NO: 1203 | SEQ ID NO: 1204 | SEQ ID NO: 1205 |
| CD70-13 | Kabat | SEQ ID NO: 1206 | SEQ ID NO: 1207 | SEQ ID NO: 1208 |
| CD70-14 | Kabat | SEQ ID NO: 1209 | SEQ ID NO: 1210 | SEQ ID NO: 1211 |
| CD70-15 | Kabat | SEQ ID NO: 1212 | SEQ ID NO: 1213 | SEQ ID NO: 1214 |
| CD70-16 | Kabat | SEQ ID NO: 1215 | SEQ ID NO: 1216 | SEQ ID NO: 1217 |
| CD70-17 | Kabat | SEQ ID NO: 1218 | SEQ ID NO: 1219 | SEQ ID NO: 1220 |
| CD70-18 | Kabat | SEQ ID NO: 1221 | SEQ ID NO: 1222 | SEQ ID NO: 1223 |
| CD70-19 | Kabat | SEQ ID NO: 1224 | SEQ ID NO: 1225 | SEQ ID NO: 1226 |
| CD70-20 | Kabat | SEQ ID NO: 1227 | SEQ ID NO: 1228 | SEQ ID NO: 1229 |
| CD70-21 | Kabat | SEQ ID NO: 1230 | SEQ ID NO: 1231 | SEQ ID NO: 1232 |
| CD70-22 | Kabat | SEQ ID NO: 1233 | SEQ ID NO: 1234 | SEQ ID NO: 1235 |
| CD70-23 | Kabat | SEQ ID NO: 1236 | SEQ ID NO: 1237 | SEQ ID NO: 1238 |
| CD70-24 | Kabat | SEQ ID NO: 1239 | SEQ ID NO: 1240 | SEQ ID NO: 1241 |
| CD70-25 | Kabat | SEQ ID NO: 1242 | SEQ ID NO: 1243 | SEQ ID NO: 1244 |
| CD70-26 | Kabat | SEQ ID NO: 1245 | SEQ ID NO: 1246 | SEQ ID NO: 1247 |
| CD70-27 | Kabat | SEQ ID NO: 1248 | SEQ ID NO: 1249 | SEQ ID NO: 1250 |
| CD70-28 | Kabat | SEQ ID NO: 1251 | SEQ ID NO: 1252 | SEQ ID NO: 1253 |
| CD70-29 | Kabat | SEQ ID NO: 1254 | SEQ ID NO: 1255 | SEQ ID NO: 1256 |
| CD70-30 | Kabat | SEQ ID NO: 1257 | SEQ ID NO: 1258 | SEQ ID NO: 1259 |
| CD70-31 | Kabat | SEQ ID NO: 1260 | SEQ ID NO: 1261 | SEQ ID NO: 1262 |
| CD70-32 | Kabat | SEQ ID NO: 1263 | SEQ ID NO: 1264 | SEQ ID NO: 1265 |
| CD70-33 | Kabat | SEQ ID NO: 1266 | SEQ ID NO: 1267 | SEQ ID NO: 1268 |
| CD70-34 | Kabat | SEQ ID NO: 1269 | SEQ ID NO: 1270 | SEQ ID NO: 1271 |
| CD70-35 | Kabat | SEQ ID NO: 1272 | SEQ ID NO: 1273 | SEQ ID NO: 1274 |
| CD70-36 | Kabat | SEQ ID NO: 1275 | SEQ ID NO: 1276 | SEQ ID NO: 1277 |
| CD70-37 | Kabat | SEQ ID NO: 1278 | SEQ ID NO: 1279 | SEQ ID NO: 1280 |
| CD70-38 | Kabat | SEQ ID NO: 1281 | SEQ ID NO: 1282 | SEQ ID NO: 1283 |
| CD70-39 | Kabat | SEQ ID NO: 1284 | SEQ ID NO: 1285 | SEQ ID NO: 1286 |
| CD70-40 | Kabat | SEQ ID NO: 1287 | SEQ ID NO: 1288 | SEQ ID NO: 1289 |
| CD70-41 | Kabat | SEQ ID NO: 1290 | SEQ ID NO: 1291 | SEQ ID NO: 1292 |
| CD70-42 | Kabat | SEQ ID NO: 1293 | SEQ ID NO: 1294 | SEQ ID NO: 1295 |
| CD70-43 | Kabat | SEQ ID NO: 1296 | SEQ ID NO: 1297 | SEQ ID NO: 1298 |
| CD70-44 | Kabat | SEQ ID NO: 1299 | SEQ ID NO: 1300 | SEQ ID NO: 1301 |
| CD70-45 | Kabat | SEQ ID NO: 1302 | SEQ ID NO: 1303 | SEQ ID NO: 1304 |
| CD70-46 | Kabat | SEQ ID NO: 1305 | SEQ ID NO: 1306 | SEQ ID NO: 1307 |
| CD70-47 | Kabat | SEQ ID NO: 1308 | SEQ ID NO: 1309 | SEQ ID NO: 1310 |
| CD70-48 | Kabat | SEQ ID NO: 1311 | SEQ ID NO: 1312 | SEQ ID NO: 1313 |
| CD70-49 | Kabat | SEQ ID NO: 1314 | SEQ ID NO: 1315 | SEQ ID NO: 1316 |
| CD70-50 | Kabat | SEQ ID NO: 1317 | SEQ ID NO: 1318 | SEQ ID NO: 1319 |
| CD70-51 | Kabat | SEQ ID NO: 1320 | SEQ ID NO: 1321 | SEQ ID NO: 1322 |
| CD70-52 | Kabat | SEQ ID NO: 1323 | SEQ ID NO: 1324 | SEQ ID NO: 1325 |
| CD70-53 | Kabat | SEQ ID NO: 1326 | SEQ ID NO: 1327 | SEQ ID NO: 1328 |
| CD70-54 | Kabat | SEQ ID NO: 1329 | SEQ ID NO: 1330 | SEQ ID NO: 1331 |
| CD70-55 | Kabat | SEQ ID NO: 1332 | SEQ ID NO: 1333 | SEQ ID NO: 1334 |
| CD70-56 | Kabat | SEQ ID NO: 1335 | SEQ ID NO: 1336 | SEQ ID NO: 1337 |
| CD70-57 | Kabat | SEQ ID NO: 1338 | SEQ ID NO: 1339 | SEQ ID NO: 1340 |
| CD70-58 | Kabat | SEQ ID NO: 1341 | SEQ ID NO: 1342 | SEQ ID NO: 1343 |
| CD70-59 | Kabat | SEQ ID NO: 1344 | SEQ ID NO: 1345 | SEQ ID NO: 1346 |
| CD70-60 | Kabat | SEQ ID NO: 1347 | SEQ ID NO: 1348 | SEQ ID NO: 1349 |
| CD70-61 | Kabat | SEQ ID NO: 1350 | SEQ ID NO: 1351 | SEQ ID NO: 1352 |
| CD70-62 | Kabat | SEQ ID NO: 1353 | SEQ ID NO: 1354 | SEQ ID NO: 1355 |
| CD70-63 | Kabat | SEQ ID NO: 1356 | SEQ ID NO: 1357 | SEQ ID NO: 1358 |
| CD70-64 | Kabat | SEQ ID NO: 1359 | SEQ ID NO: 1360 | SEQ ID NO: 1361 |
| CD70-65 | Kabat | SEQ ID NO: 1362 | SEQ ID NO: 1363 | SEQ ID NO: 1364 |
| CD70-66 | Kabat | SEQ ID NO: 1365 | SEQ ID NO: 1366 | SEQ ID NO: 1367 |
| CD70-67 | Kabat | SEQ ID NO: 1368 | SEQ ID NO: 1369 | SEQ ID NO: 1370 |
| CD70-68 | Kabat | SEQ ID NO: 1371 | SEQ ID NO: 1372 | SEQ ID NO: 1373 |
| CD70-69 | Kabat | SEQ ID NO: 1374 | SEQ ID NO: 1375 | SEQ ID NO: 1376 |
| CD70-70 | Kabat | SEQ ID NO: 1377 | SEQ ID NO: 1378 | SEQ ID NO: 1379 |
| CD70-71 | Kabat | SEQ ID NO: 1380 | SEQ ID NO: 1381 | SEQ ID NO: 1382 |
| CD70-72 | Kabat | SEQ ID NO: 1383 | SEQ ID NO: 1384 | SEQ ID NO: 1385 |
| CD70-73 | Kabat | SEQ ID NO: 1386 | SEQ ID NO: 1387 | SEQ ID NO: 1388 |
| CD70-74 | Kabat | SEQ ID NO: 1389 | SEQ ID NO: 1390 | SEQ ID NO: 1391 |
| CD70-75 | Kabat | SEQ ID NO: 1392 | SEQ ID NO: 1393 | SEQ ID NO: 1394 |
| CD70-76 | Kabat | SEQ ID NO: 1395 | SEQ ID NO: 1396 | SEQ ID NO: 1397 |
| CD70-77 | Kabat | SEQ ID NO: 1398 | SEQ ID NO: 1399 | SEQ ID NO: 1400 |
| CD70-78 | Kabat | SEQ ID NO: 1401 | SEQ ID NO: 1402 | SEQ ID NO: 1403 |
| CD70-79 | Kabat | SEQ ID NO: 1404 | SEQ ID NO: 1405 | SEQ ID NO: 1406 |
| CD70-80 | Kabat | SEQ ID NO: 1407 | SEQ ID NO: 1408 | SEQ ID NO: 1409 |
| CD70-81 | Kabat | SEQ ID NO: 1410 | SEQ ID NO: 1411 | SEQ ID NO: 1412 |
| CD70-82 | Kabat | SEQ ID NO: 1413 | SEQ ID NO: 1414 | SEQ ID NO: 1415 |
| CD70-83 | Kabat | SEQ ID NO: 1416 | SEQ ID NO: 1417 | SEQ ID NO: 1418 |
| CD70-84 | Kabat | SEQ ID NO: 1419 | SEQ ID NO: 1420 | SEQ ID NO: 1421 |
| CD70-85 | Kabat | SEQ ID NO: 1422 | SEQ ID NO: 1423 | SEQ ID NO: 1424 |
| CD70-86 | Kabat | SEQ ID NO: 1425 | SEQ ID NO: 1426 | SEQ ID NO: 1427 |
| CD70-87 | Kabat | SEQ ID NO: 1428 | SEQ ID NO: 1429 | SEQ ID NO: 1430 |
| CD70-88 | Kabat | SEQ ID NO: 1431 | SEQ ID NO: 1432 | SEQ ID NO: 1433 |
| CD70-89 | Kabat | SEQ ID NO: 1434 | SEQ ID NO: 1435 | SEQ ID NO: 1436 |
| CD70-90 | Kabat | SEQ ID NO: 1437 | SEQ ID NO: 1438 | SEQ ID NO: 1439 |
| CD70-91 | Kabat | SEQ ID NO: 1440 | SEQ ID NO: 1441 | SEQ ID NO: 1442 |
| CD70-92 | Kabat | SEQ ID NO: 1443 | SEQ ID NO: 1444 | SEQ ID NO: 1445 |
| CD70-93 | Kabat | SEQ ID NO: 1446 | SEQ ID NO: 1447 | SEQ ID NO: 1448 |
| CD70-94 | Kabat | SEQ ID NO: 1449 | SEQ ID NO: 1450 | SEQ ID NO: 1451 |
| CD70-95 | Kabat | SEQ ID NO: 1452 | SEQ ID NO: 1453 | SEQ ID NO: 1454 |
| CD70-96 | Kabat | SEQ ID NO: 1455 | SEQ ID NO: 1456 | SEQ ID NO: 1457 |
| CD70-97 | Kabat | SEQ ID NO: 1458 | SEQ ID NO: 1459 | SEQ ID NO: 1460 |
| CD70-98 | Kabat | SEQ ID NO: 1461 | SEQ ID NO: 1462 | SEQ ID NO: 1463 |
| CD70-99 | Kabat | SEQ ID NO: 1464 | SEQ ID NO: 1465 | SEQ ID NO: 1466 |
| CD70-100 | Kabat | SEQ ID NO: 1467 | SEQ ID NO: 1468 | SEQ ID NO: 1469 |
| CD70-101 | Kabat | SEQ ID NO: 1470 | SEQ ID NO: 1471 | SEQ ID NO: 1472 |
| CD70-102 | Kabat | SEQ ID NO: 1473 | SEQ ID NO: 1474 | SEQ ID NO: 1475 |
| CD70-103 | Kabat | SEQ ID NO: 1476 | SEQ ID NO: 1477 | SEQ ID NO: 1478 |
| CD70-104 | Kabat | SEQ ID NO: 1479 | SEQ ID NO: 1480 | SEQ ID NO: 1481 |
| CD70-105 | Kabat | SEQ ID NO: 1482 | SEQ ID NO: 1483 | SEQ ID NO: 1484 |
| CD70-106 | Kabat | SEQ ID NO: 1485 | SEQ ID NO: 1486 | SEQ ID NO: 1487 |
| CD70-107 | Kabat | SEQ ID NO: 1488 | SEQ ID NO: 1489 | SEQ ID NO: 1490 |
| CD70-108 | Kabat | SEQ ID NO: 1491 | SEQ ID NO: 1492 | SEQ ID NO: 1493 |
| CD70-109 | Kabat | SEQ ID NO: 1494 | SEQ ID NO: 1495 | SEQ ID NO: 1496 |
| CD70-110 | Kabat | SEQ ID NO: 1497 | SEQ ID NO: 1498 | SEQ ID NO: 1499 |
| CD70-111 | Kabat | SEQ ID NO: 1500 | SEQ ID NO: 1501 | SEQ ID NO: 1502 |
| CD70-112 | Kabat | SEQ ID NO: 1503 | SEQ ID NO: 1504 | SEQ ID NO: 1505 |
| CD70-113 | Kabat | SEQ ID NO: 1506 | SEQ ID NO: 1507 | SEQ ID NO: 1508 |
| CD70-114 | Kabat | SEQ ID NO: 1509 | SEQ ID NO: 1510 | SEQ ID NO: 1511 |
| CD70-115 | Kabat | SEQ ID NO: 1512 | SEQ ID NO: 1513 | SEQ ID NO: 1514 |
| CD70-116 | Kabat | SEQ ID NO: 1515 | SEQ ID NO: 1516 | SEQ ID NO: 1517 |
| CD70-117 | Kabat | SEQ ID NO: 1518 | SEQ ID NO: 1519 | SEQ ID NO: 1520 |
| CD70-118 | Kabat | SEQ ID NO: 1521 | SEQ ID NO: 1522 | SEQ ID NO: 1523 |
| CD70-119 | Kabat | SEQ ID NO: 1524 | SEQ ID NO: 1525 | SEQ ID NO: 1526 |
| CD70-120 | Kabat | SEQ ID NO: 1527 | SEQ ID NO: 1528 | SEQ ID NO: 1529 |
| CD70-121 | Kabat | SEQ ID NO: 1530 | SEQ ID NO: 1531 | SEQ ID NO: 1532 |
| CD70-122 | Kabat | SEQ ID NO: 1533 | SEQ ID NO: 1534 | SEQ ID NO: 1535 |
| CD70-123 | Kabat | SEQ ID NO: 1536 | SEQ ID NO: 1537 | SEQ ID NO: 1538 |
| CD70-124 | Kabat | SEQ ID NO: 1539 | SEQ ID NO: 1540 | SEQ ID NO: 1541 |
| CD70-125 | Kabat | SEQ ID NO: 1542 | SEQ ID NO: 1543 | SEQ ID NO: 1544 |
| CD70-126 | Kabat | SEQ ID NO: 1545 | SEQ ID NO: 1546 | SEQ ID NO: 1547 |
| CD70-127 | Kabat | SEQ ID NO: 1548 | SEQ ID NO: 1549 | SEQ ID NO: 1550 |
| CD70-128 | Kabat | SEQ ID NO: 1551 | SEQ ID NO: 1552 | SEQ ID NO: 1553 |
| CD70-129 | Kabat | SEQ ID NO: 1554 | SEQ ID NO: 1555 | SEQ ID NO: 1556 |
| CD70-130 | Kabat | SEQ ID NO: 1557 | SEQ ID NO: 1558 | SEQ ID NO: 1559 |
| CD70-131 | Kabat | SEQ ID NO: 1560 | SEQ ID NO: 1561 | SEQ ID NO: 1562 |
| CD70-132 | Kabat | SEQ ID NO: 1563 | SEQ ID NO: 1564 | SEQ ID NO: 1565 |
| CD70-133 | Kabat | SEQ ID NO: 1566 | SEQ ID NO: 1567 | SEQ ID NO: 1568 |
| CD70-134 | Kabat | SEQ ID NO: 1569 | SEQ ID NO: 1570 | SEQ ID NO: 1571 |
| CD70-135 | Kabat | SEQ ID NO: 1572 | SEQ ID NO: 1573 | SEQ ID NO: 1574 |
| CD70-136 | Kabat | SEQ ID NO: 1575 | SEQ ID NO: 1576 | SEQ ID NO: 1577 |
| CD70-137 | Kabat | SEQ ID NO: 1578 | SEQ ID NO: 1579 | SEQ ID NO: 1580 |
| CD70-138 | Kabat | SEQ ID NO: 1581 | SEQ ID NO: 1582 | SEQ ID NO: 1583 |
| CD70-139 | Kabat | SEQ ID NO: 1584 | SEQ ID NO: 1585 | SEQ ID NO: 1586 |
| CD70-140 | Kabat | SEQ ID NO: 1587 | SEQ ID NO: 1588 | SEQ ID NO: 1589 |
| CD70-141 | Kabat | SEQ ID NO: 1590 | SEQ ID NO: 1591 | SEQ ID NO: 1592 |
| CD70-142 | Kabat | SEQ ID NO: 1593 | SEQ ID NO: 1594 | SEQ ID NO: 1595 |
| CD70-143 | Kabat | SEQ ID NO: 1596 | SEQ ID NO: 1597 | SEQ ID NO: 1598 |
| CD70-144 | Kabat | SEQ ID NO: 1599 | SEQ ID NO: 1600 | SEQ ID NO: 1601 |
| CD70-145 | Kabat | SEQ ID NO: 1602 | SEQ ID NO: 1603 | SEQ ID NO: 1604 |
| CD70-146 | Kabat | SEQ ID NO: 1605 | SEQ ID NO: 1606 | SEQ ID NO: 1607 |
| CD70-147 | Kabat | SEQ ID NO: 1608 | SEQ ID NO: 1609 | SEQ ID NO: 1610 |
| CD70-148 | Kabat | SEQ ID NO: 1611 | SEQ ID NO: 1612 | SEQ ID NO: 1613 |
| CD70-149 | Kabat | SEQ ID NO: 1614 | SEQ ID NO: 1615 | SEQ ID NO: 1616 |
| CD70-150 | Kabat | SEQ ID NO: 1617 | SEQ ID NO: 1618 | SEQ ID NO: 1619 |
| CD70-151 | Kabat | SEQ ID NO: 1620 | SEQ ID NO: 1621 | SEQ ID NO: 1622 |
| CD70-152 | Kabat | SEQ ID NO: 1623 | SEQ ID NO: 1624 | SEQ ID NO: 1625 |
| CD70-153 | Kabat | SEQ ID NO: 1626 | SEQ ID NO: 1627 | SEQ ID NO: 1628 |
| CD70-154 | Kabat | SEQ ID NO: 1629 | SEQ ID NO: 1630 | SEQ ID NO: 1631 |
| CD70-155 | Kabat | SEQ ID NO: 1632 | SEQ ID NO: 1633 | SEQ ID NO: 1634 |
| CD70-156 | Kabat | SEQ ID NO: 1635 | SEQ ID NO: 1636 | SEQ ID NO: 1637 |
| CD70-157 | Kabat | SEQ ID NO: 1638 | SEQ ID NO: 1639 | SEQ ID NO: 1640 |
| CD70-158 | Kabat | SEQ ID NO: 1641 | SEQ ID NO: 1642 | SEQ ID NO: 1643 |
| CD70-159 | Kabat | SEQ ID NO: 1644 | SEQ ID NO: 1645 | SEQ ID NO: 1646 |
| CD70-160 | Kabat | SEQ ID NO: 1647 | SEQ ID NO: 1648 | SEQ ID NO: 1649 |
| CD70-161 | Kabat | SEQ ID NO: 1650 | SEQ ID NO: 1651 | SEQ ID NO: 1652 |
| CD70-162 | Kabat | SEQ ID NO: 1653 | SEQ ID NO: 1654 | SEQ ID NO: 1655 |
| CD70-163 | Kabat | SEQ ID NO: 1656 | SEQ ID NO: 1657 | SEQ ID NO: 1658 |
| CD70-164 | Kabat | SEQ ID NO: 1659 | SEQ ID NO: 1660 | SEQ ID NO: 1661 |
| CD70-165 | Kabat | SEQ ID NO: 1662 | SEQ ID NO: 1663 | SEQ ID NO: 1664 |
| CD70-166 | Kabat | SEQ ID NO: 1665 | SEQ ID NO: 1666 | SEQ ID NO: 1667 |
| CD70-167 | Kabat | SEQ ID NO: 1668 | SEQ ID NO: 1669 | SEQ ID NO: 1670 |
| CD70-168 | Kabat | SEQ ID NO: 1671 | SEQ ID NO: 1672 | SEQ ID NO: 1673 |
| CD70-169 | Kabat | SEQ ID NO: 1674 | SEQ ID NO: 1675 | SEQ ID NO: 1676 |
| CD70-170 | Kabat | SEQ ID NO: 1677 | SEQ ID NO: 1678 | SEQ ID NO: 1679 |
| CD70-171 | Kabat | SEQ ID NO: 1680 | SEQ ID NO: 1681 | SEQ ID NO: 1682 |
| CD70-172 | Kabat | SEQ ID NO: 1683 | SEQ ID NO: 1684 | SEQ ID NO: 1685 |
| CD70-173 | Kabat | SEQ ID NO: 1686 | SEQ ID NO: 1687 | SEQ ID NO: 1688 |
| CD70-174 | Kabat | SEQ ID NO: 1689 | SEQ ID NO: 1690 | SEQ ID NO: 1691 |
| CD70-175 | Kabat | SEQ ID NO: 1692 | SEQ ID NO: 1693 | SEQ ID NO: 1694 |
| CD70-176 | Kabat | SEQ ID NO: 1695 | SEQ ID NO: 1696 | SEQ ID NO: 1697 |
| CD70-177 | Kabat | SEQ ID NO: 1698 | SEQ ID NO: 1699 | SEQ ID NO: 1700 |
| CD70-178 | Kabat | SEQ ID NO: 1701 | SEQ ID NO: 1702 | SEQ ID NO: 1703 |
| CD70-179 | Kabat | SEQ ID NO: 1704 | SEQ ID NO: 1705 | SEQ ID NO: 1706 |
| CD70-180 | Kabat | SEQ ID NO: 1707 | SEQ ID NO: 1708 | SEQ ID NO: 1709 |
| 1C2 | Kabat | SEQ ID NO: 1710 | SEQ ID NO: 1711 | SEQ ID NO: 1712 |
| 9D1 | Kabat | SEQ ID NO: 1713 | SEQ ID NO: 1714 | SEQ ID NO: 1715 |
| 8B12 | Kabat | SEQ ID NO: 1716 | SEQ ID NO: 1717 | SEQ ID NO: 1718 |
| 8C12 | Kabat | SEQ ID NO: 1719 | SEQ ID NO: 1720 | SEQ ID NO: 1721 |
| 9E1 | Kabat | SEQ ID NO: 1722 | SEQ ID NO: 1723 | SEQ ID NO: 1724 |
| 5F4 | Kabat | SEQ ID NO: 1725 | SEQ ID NO: 1726 | SEQ ID NO: 1727 |
| 5B2 | Kabat | SEQ ID NO: 1728 | SEQ ID NO: 1729 | SEQ ID NO: 1730 |
| 6D5 | Kabat | SEQ ID NO: 1731 | SEQ ID NO: 1732 | SEQ ID NO: 1733 |
| 4D2 | Kabat | SEQ ID NO: 1734 | SEQ ID NO: 1735 | SEQ ID NO: 1736 |
| 9A1 | Kabat | SEQ ID NO: 1737 | SEQ ID NO: 1738 | SEQ ID NO: 1739 |
| 9G2 | Kabat | SEQ ID NO: 1740 | SEQ ID NO: 1741 | SEQ ID NO: 1742 |
| 9B2 | Kabat | SEQ ID NO: 1743 | SEQ ID NO: 1744 | SEQ ID NO: 1745 |
| 24E3 | Kabat | SEQ ID NO: 1746 | SEQ ID NO: 1747 | SEQ ID NO: 1748 |
| 33D8 | Kabat | SEQ ID NO: 1749 | SEQ ID NO: 1750 | SEQ ID NO: 1751 |
| 24F2 | Kabat | SEQ ID NO: 1752 | SEQ ID NO: 1753 | SEQ ID NO: 1754 |
| 24B6 | Kabat | SEQ ID NO: 1755 | SEQ ID NO: 1756 | SEQ ID NO: 1757 |
| 19G10 | Kabat | SEQ ID NO: 1758 | SEQ ID NO: 1759 | SEQ ID NO: 1760 |
| 45B12 | Kabat | SEQ ID NO: 1761 | SEQ ID NO: 1762 | SEQ ID NO: 1763 |
| 45D9 | Kabat | SEQ ID NO: 1764 | SEQ ID NO: 1765 | SEQ ID NO: 1766 |
| 45F8 | Kabat | SEQ ID NO: 1767 | SEQ ID NO: 1768 | SEQ ID NO: 1769 |
| 45A12 | Kabat | SEQ ID NO: 1770 | SEQ ID NO: 1771 | SEQ ID NO: 1772 |
| 45B6 | Kabat | SEQ ID NO: 1773 | SEQ ID NO: 1774 | SEQ ID NO: 1775 |
| 57B6 | Kabat | SEQ ID NO: 1776 | SEQ ID NO: 1777 | SEQ ID NO: 1778 |
| 59D10 | Kabat | SEQ ID NO: 1779 | SEQ ID NO: 1780 | SEQ ID NO: 1781 |
| 27B3 | Kabat | SEQ ID NO: 1782 | SEQ ID NO: 1783 | SEQ ID NO: 1784 |
| 36A9 | Kabat | SEQ ID NO: 1785 | SEQ ID NO: 1786 | SEQ ID NO: 1787 |
| 53F1 | Kabat | SEQ ID NO: 1788 | SEQ ID NO: 1789 | SEQ ID NO: 1790 |
| 36D6 | Kabat | SEQ ID NO: 1791 | SEQ ID NO: 1792 | SEQ ID NO: 1793 |
| 53G1 | Kabat | SEQ ID NO: 1794 | SEQ ID NO: 1795 | SEQ ID NO: 1796 |
| 35G3 | Kabat | SEQ ID NO: 1797 | SEQ ID NO: 1798 | SEQ ID NO: 1799 |
| 53C1 | Kabat | SEQ ID NO: 1800 | SEQ ID NO: 1801 | SEQ ID NO: 1802 |
| 35F6 | Kabat | SEQ ID NO: 1803 | SEQ ID NO: 1804 | SEQ ID NO: 1805 |
| 36G2 | Kabat | SEQ ID NO: 1806 | SEQ ID NO: 1807 | SEQ ID NO: 1808 |
| 39D5 | Kabat | SEQ ID NO: 1809 | SEQ ID NO: 1810 | SEQ ID NO: 1811 |
| 42D12 | Kabat | SEQ ID NO: 1812 | SEQ ID NO: 1813 | SEQ ID NO: 1814 |
| 35C1 | Kabat | SEQ ID NO: 1815 | SEQ ID NO: 1816 | SEQ ID NO: 1817 |
| 41D12 | Kabat | SEQ ID NO: 1818 | SEQ ID NO: 1819 | SEQ ID NO: 1820 |
| 41H8 | Kabat | SEQ ID NO: 1821 | SEQ ID NO: 1822 | SEQ ID NO: 1823 |
| 35G2 | Kabat | SEQ ID NO: 1824 | SEQ ID NO: 1825 | SEQ ID NO: 1826 |
| 40F1 | Kabat | SEQ ID NO: 1827 | SEQ ID NO: 1828 | SEQ ID NO: 1829 |
| 53B1 | Kabat | SEQ ID NO: 1830 | SEQ ID NO: 1831 | SEQ ID NO: 1832 |
| 39C3 | Kabat | SEQ ID NO: 1833 | SEQ ID NO: 1834 | SEQ ID NO: 1835 |
| 53D1 | Kabat | SEQ ID NO: 1836 | SEQ ID NO: 1837 | SEQ ID NO: 1838 |
| 53H1 | Kabat | SEQ ID NO: 1839 | SEQ ID NO: 1840 | SEQ ID NO: 1841 |
| 53A2 | Kabat | SEQ ID NO: 1842 | SEQ ID NO: 1843 | SEQ ID NO: 1844 |
| ARGX-110 | Kabat | SEQ ID NO: 1845 | SEQ ID NO: 1846 | SEQ ID NO: 1847 |
| CTX-130 | Kabat | SEQ ID NO: 1848 | SEQ ID NO: 1849 | SEQ ID NO: 1850 |
| CTX-130 | Kabat | SEQ ID NO: 1851 | SEQ ID NO: 1852 | SEQ ID NO: 1853 |
| 4SCAR70 | Kabat | SEQ ID NO: 1854 | SEQ ID NO: 1855 | SEQ ID NO: 1856 |

TABLE 3
Exemplary light chain complementarity determining
regions of anti-CD70 antibodies

| Antibody ID | CDRL1 | CDRL2 | CDRL3 |
|---|---|---|---|
| 31H1 | SEQ ID NO: 478 | SEQ ID NO: 479 | SEQ ID NO: 480 |
| 63B2 | SEQ ID NO: 481 | SEQ ID NO: 482 | SEQ ID NO: 483 |
| 40E3 | SEQ ID NO: 484 | SEQ ID NO: 485 | SEQ ID NO: 486 |
| 42C3 | SEQ ID NO: 487 | SEQ ID NO: 488 | SEQ ID NO: 489 |
| 45F11 | SEQ ID NO: 490 | SEQ ID NO: 491 | SEQ ID NO: 492 |
| 64F9 | SEQ ID NO: 493 | SEQ ID NO: 494 | SEQ ID NO: 495 |
| 72C2 | SEQ ID NO: 496 | SEQ ID NO: 497 | SEQ ID NO: 498 |
| 2F10 | SEQ ID NO: 499 | SEQ ID NO: 500 | SEQ ID NO: 501 |
| 4F11 | SEQ ID NO: 502 | SEQ ID NO: 503 | SEQ ID NO: 504 |
| 10H10 | SEQ ID NO: 505 | SEQ ID NO: 506 | SEQ ID NO: 507 |
| 17G6 | SEQ ID NO: 508 | SEQ ID NO: 509 | SEQ ID NO: 510 |
| 65E11 | SEQ ID NO: 511 | SEQ ID NO: 512 | SEQ ID NO: 513 |
| P02B10 | SEQ ID NO: 514 | SEQ ID NO: 515 | SEQ ID NO: 516 |
| P07D03 | SEQ ID NO: 517 | SEQ ID NO: 518 | SEQ ID NO: 519 |
| P08A02 | SEQ ID NO: 520 | SEQ ID NO: 521 | SEQ ID NO: 522 |
| P08E02 | SEQ ID NO: 523 | SEQ ID NO: 524 | SEQ ID NO: 525 |
| P08F08 | SEQ ID NO: 526 | SEQ ID NO: 527 | SEQ ID NO: 528 |
| P08G02 | SEQ ID NO: 529 | SEQ ID NO: 530 | SEQ ID NO: 531 |
| P12B09 | SEQ ID NO: 532 | SEQ ID NO: 533 | SEQ ID NO: 534 |
| P12F02 | SEQ ID NO: 535 | SEQ ID NO: 536 | SEQ ID NO: 537 |
| P12G07 | SEQ ID NO: 538 | SEQ ID NO: 539 | SEQ ID NO: 540 |
| P13F04 | SEQ ID NO: 541 | SEQ ID NO: 542 | SEQ ID NO: 543 |
| P15D02 | SEQ ID NO: 544 | SEQ ID NO: 545 | SEQ ID NO: 546 |
| P16C05 | SEQ ID NO: 547 | SEQ ID NO: 548 | SEQ ID NO: 549 |
| 10A1 | SEQ ID NO: 550 | SEQ ID NO: 551 | SEQ ID NO: 552 |
| 10E2 | SEQ ID NO: 553 | SEQ ID NO: 554 | SEQ ID NO: 555 |
| 11A1 | SEQ ID NO: 556 | SEQ ID NO: 557 | SEQ ID NO: 558 |
| 11C1 | SEQ ID NO: 559 | SEQ ID NO: 560 | SEQ ID NO: 561 |
| 11D1 | SEQ ID NO: 562 | SEQ ID NO: 563 | SEQ ID NO: 564 |
| 11E1 | SEQ ID NO: 565 | SEQ ID NO: 566 | SEQ ID NO: 567 |
| 12A2 | SEQ ID NO: 568 | SEQ ID NO: 569 | SEQ ID NO: 570 |
| 12C4 | SEQ ID NO: 571 | SEQ ID NO: 572 | SEQ ID NO: 573 |
| 12C5 | SEQ ID NO: 574 | SEQ ID NO: 575 | SEQ ID NO: 576 |
| 12D3 | SEQ ID NO: 577 | SEQ ID NO: 578 | SEQ ID NO: 579 |
| 12D6 | SEQ ID NO: 580 | SEQ ID NO: 581 | SEQ ID NO: 582 |
| 12D7 | SEQ ID NO: 583 | SEQ ID NO: 584 | SEQ ID NO: 585 |
| 12F5 | SEQ ID NO: 586 | SEQ ID NO: 587 | SEQ ID NO: 588 |
| 12H4 | SEQ ID NO: 589 | SEQ ID NO: 590 | SEQ ID NO: 591 |
| 8C8 | SEQ ID NO: 592 | SEQ ID NO: 593 | SEQ ID NO: 594 |
| 8F7 | SEQ ID NO: 595 | SEQ ID NO: 596 | SEQ ID NO: 597 |
| 8F8 | SEQ ID NO: 598 | SEQ ID NO: 599 | SEQ ID NO: 600 |
| 9D8 | SEQ ID NO: 601 | SEQ ID NO: 602 | SEQ ID NO: 603 |
| 9E10 | SEQ ID NO: 604 | SEQ ID NO: 605 | SEQ ID NO: 606 |
| 9E5 | SEQ ID NO: 607 | SEQ ID NO: 608 | SEQ ID NO: 609 |
| 9F4 | SEQ ID NO: 610 | SEQ ID NO: 611 | SEQ ID NO: 612 |
| 9F8 | SEQ ID NO: 613 | SEQ ID NO: 614 | SEQ ID NO: 615 |
| 12C6 | SEQ ID NO: 616 | SEQ ID NO: 617 | SEQ ID NO: 618 |
| CD70-1 | SEQ ID NO: 1857 | SEQ ID NO: 1858 | SEQ ID NO: 1859 |
| CD70-2 | SEQ ID NO: 1860 | SEQ ID NO: 1861 | SEQ ID NO: 1862 |
| CD70-3 | SEQ ID NO: 1863 | SEQ ID NO: 1864 | SEQ ID NO: 1865 |
| CD70-4 | SEQ ID NO: 1866 | SEQ ID NO: 1867 | SEQ ID NO: 1868 |
| CD70-5 | SEQ ID NO: 1869 | SEQ ID NO: 1870 | SEQ ID NO: 1871 |
| CD70-6 | SEQ ID NO: 1872 | SEQ ID NO: 1873 | SEQ ID NO: 1874 |
| CD70-7 | SEQ ID NO: 1875 | SEQ ID NO: 1876 | SEQ ID NO: 1877 |
| CD70-8 | SEQ ID NO: 1878 | SEQ ID NO: 1879 | SEQ ID NO: 1880 |
| CD70-9 | SEQ ID NO: 1881 | SEQ ID NO: 1882 | SEQ ID NO: 1883 |
| CD70-10 | SEQ ID NO: 1884 | SEQ ID NO: 1885 | SEQ ID NO: 1886 |
| CD70-11 | SEQ ID NO: 1887 | SEQ ID NO: 1888 | SEQ ID NO: 1889 |
| CD70-12 | SEQ ID NO: 1890 | SEQ ID NO: 1891 | SEQ ID NO: 1892 |
| CD70-13 | SEQ ID NO: 1893 | SEQ ID NO: 1894 | SEQ ID NO: 1895 |
| CD70-14 | SEQ ID NO: 1896 | SEQ ID NO: 1897 | SEQ ID NO: 1898 |
| CD70-15 | SEQ ID NO: 1899 | SEQ ID NO: 1900 | SEQ ID NO: 1901 |
| CD70-16 | SEQ ID NO: 1902 | SEQ ID NO: 1903 | SEQ ID NO: 1904 |
| CD70-17 | SEQ ID NO: 1905 | SEQ ID NO: 1906 | SEQ ID NO: 1907 |
| CD70-18 | SEQ ID NO: 1908 | SEQ ID NO: 1909 | SEQ ID NO: 1910 |
| CD70-19 | SEQ ID NO: 1911 | SEQ ID NO: 1912 | SEQ ID NO: 1913 |
| CD70-20 | SEQ ID NO: 1914 | SEQ ID NO: 1915 | SEQ ID NO: 1916 |
| CD70-21 | SEQ ID NO: 1917 | SEQ ID NO: 1918 | SEQ ID NO: 1919 |
| CD70-22 | SEQ ID NO: 1920 | SEQ ID NO: 1921 | SEQ ID NO: 1922 |
| CD70-23 | SEQ ID NO: 1923 | SEQ ID NO: 1924 | SEQ ID NO: 1925 |
| CD70-24 | SEQ ID NO: 1926 | SEQ ID NO: 1927 | SEQ ID NO: 1928 |
| CD70-25 | SEQ ID NO: 1929 | SEQ ID NO: 1930 | SEQ ID NO: 1931 |
| CD70-26 | SEQ ID NO: 1932 | SEQ ID NO: 1933 | SEQ ID NO: 1934 |
| CD70-27 | SEQ ID NO: 1935 | SEQ ID NO: 1936 | SEQ ID NO: 1937 |
| CD70-28 | SEQ ID NO: 1938 | SEQ ID NO: 1939 | SEQ ID NO: 1940 |
| CD70-29 | SEQ ID NO: 1941 | SEQ ID NO: 1942 | SEQ ID NO: 1943 |
| CD70-30 | SEQ ID NO: 1944 | SEQ ID NO: 1945 | SEQ ID NO: 1946 |
| CD70-31 | SEQ ID NO: 1947 | SEQ ID NO: 1948 | SEQ ID NO: 1949 |
| CD70-32 | SEQ ID NO: 1950 | SEQ ID NO: 1951 | SEQ ID NO: 1952 |
| CD70-33 | SEQ ID NO: 1953 | SEQ ID NO: 1954 | SEQ ID NO: 1955 |
| CD70-34 | SEQ ID NO: 1956 | SEQ ID NO: 1957 | SEQ ID NO: 1958 |
| CD70-35 | SEQ ID NO: 1959 | SEQ ID NO: 1960 | SEQ ID NO: 1961 |
| CD70-36 | SEQ ID NO: 1962 | SEQ ID NO: 1963 | SEQ ID NO: 1964 |
| CD70-37 | SEQ ID NO: 1965 | SEQ ID NO: 1966 | SEQ ID NO: 1967 |
| CD70-38 | SEQ ID NO: 1968 | SEQ ID NO: 1969 | SEQ ID NO: 1970 |
| CD70-39 | SEQ ID NO: 1971 | SEQ ID NO: 1972 | SEQ ID NO: 1973 |
| CD70-40 | SEQ ID NO: 1974 | SEQ ID NO: 1975 | SEQ ID NO: 1976 |
| CD70-41 | SEQ ID NO: 1977 | SEQ ID NO: 1978 | SEQ ID NO: 1979 |
| CD70-42 | SEQ ID NO: 1980 | SEQ ID NO: 1981 | SEQ ID NO: 1982 |
| CD70-43 | SEQ ID NO: 1983 | SEQ ID NO: 1984 | SEQ ID NO: 1985 |
| CD70-44 | SEQ ID NO: 1986 | SEQ ID NO: 1987 | SEQ ID NO: 1988 |
| CD70-45 | SEQ ID NO: 1989 | SEQ ID NO: 1990 | SEQ ID NO: 1991 |
| CD70-46 | SEQ ID NO: 1992 | SEQ ID NO: 1993 | SEQ ID NO: 1994 |
| CD70-47 | SEQ ID NO: 1995 | SEQ ID NO: 1996 | SEQ ID NO: 1997 |
| CD70-48 | SEQ ID NO: 1998 | SEQ ID NO: 1999 | SEQ ID NO: 2000 |
| CD70-49 | SEQ ID NO: 2001 | SEQ ID NO: 2002 | SEQ ID NO: 2003 |
| CD70-50 | SEQ ID NO: 2004 | SEQ ID NO: 2005 | SEQ ID NO: 2006 |
| CD70-51 | SEQ ID NO: 2007 | SEQ ID NO: 2008 | SEQ ID NO: 2009 |
| CD70-52 | SEQ ID NO: 2010 | SEQ ID NO: 2011 | SEQ ID NO: 2012 |
| CD70-53 | SEQ ID NO: 2013 | SEQ ID NO: 2014 | SEQ ID NO: 2015 |
| CD70-54 | SEQ ID NO: 2016 | SEQ ID NO: 2017 | SEQ ID NO: 2018 |
| CD70-55 | SEQ ID NO: 2019 | SEQ ID NO: 2020 | SEQ ID NO: 2021 |
| CD70-56 | SEQ ID NO: 2022 | SEQ ID NO: 2023 | SEQ ID NO: 2024 |
| CD70-57 | SEQ ID NO: 2025 | SEQ ID NO: 2026 | SEQ ID NO: 2027 |
| CD70-58 | SEQ ID NO: 2028 | SEQ ID NO: 2029 | SEQ ID NO: 2030 |
| CD70-59 | SEQ ID NO: 2031 | SEQ ID NO: 2032 | SEQ ID NO: 2033 |
| CD70-60 | SEQ ID NO: 2034 | SEQ ID NO: 2035 | SEQ ID NO: 2036 |
| CD70-61 | SEQ ID NO: 2037 | SEQ ID NO: 2038 | SEQ ID NO: 2039 |
| CD70-62 | SEQ ID NO: 2040 | SEQ ID NO: 2041 | SEQ ID NO: 2042 |
| CD70-63 | SEQ ID NO: 2043 | SEQ ID NO: 2044 | SEQ ID NO: 2045 |
| CD70-64 | SEQ ID NO: 2046 | SEQ ID NO: 2047 | SEQ ID NO: 2048 |
| CD70-65 | SEQ ID NO: 2049 | SEQ ID NO: 2050 | SEQ ID NO: 2051 |
| CD70-66 | SEQ ID NO: 2052 | SEQ ID NO: 2053 | SEQ ID NO: 2054 |
| CD70-67 | SEQ ID NO: 2055 | SEQ ID NO: 2056 | SEQ ID NO: 2057 |
| CD70-68 | SEQ ID NO: 2058 | SEQ ID NO: 2059 | SEQ ID NO: 2060 |
| CD70-69 | SEQ ID NO: 2061 | SEQ ID NO: 2062 | SEQ ID NO: 2063 |
| CD70-70 | SEQ ID NO: 2064 | SEQ ID NO: 2065 | SEQ ID NO: 2066 |
| CD70-71 | SEQ ID NO: 2067 | SEQ ID NO: 2068 | SEQ ID NO: 2069 |
| CD70-72 | SEQ ID NO: 2070 | SEQ ID NO: 2071 | SEQ ID NO: 2072 |
| CD70-73 | SEQ ID NO: 2073 | SEQ ID NO: 2074 | SEQ ID NO: 2075 |
| CD70-74 | SEQ ID NO: 2076 | SEQ ID NO: 2077 | SEQ ID NO: 2078 |
| CD70-75 | SEQ ID NO: 2079 | SEQ ID NO: 2080 | SEQ ID NO: 2081 |
| CD70-76 | SEQ ID NO: 2082 | SEQ ID NO: 2083 | SEQ ID NO: 2084 |
| CD70-77 | SEQ ID NO: 2085 | SEQ ID NO: 2086 | SEQ ID NO: 2087 |
| CD70-78 | SEQ ID NO: 2088 | SEQ ID NO: 2089 | SEQ ID NO: 2090 |
| CD70-79 | SEQ ID NO: 2091 | SEQ ID NO: 2092 | SEQ ID NO: 2093 |
| CD70-80 | SEQ ID NO: 2094 | SEQ ID NO: 2095 | SEQ ID NO: 2096 |
| CD70-81 | SEQ ID NO: 2097 | SEQ ID NO: 2098 | SEQ ID NO: 2099 |
| CD70-82 | SEQ ID NO: 2100 | SEQ ID NO: 2101 | SEQ ID NO: 2102 |
| CD70-83 | SEQ ID NO: 2103 | SEQ ID NO: 2104 | SEQ ID NO: 2105 |
| CD70-84 | SEQ ID NO: 2106 | SEQ ID NO: 2107 | SEQ ID NO: 2108 |
| CD70-85 | SEQ ID NO: 2109 | SEQ ID NO: 2110 | SEQ ID NO: 2111 |
| CD70-86 | SEQ ID NO: 2112 | SEQ ID NO: 2113 | SEQ ID NO: 2114 |
| CD70-87 | SEQ ID NO: 2115 | SEQ ID NO: 2116 | SEQ ID NO: 2117 |
| CD70-88 | SEQ ID NO: 2118 | SEQ ID NO: 2119 | SEQ ID NO: 2120 |
| CD70-89 | SEQ ID NO: 2121 | SEQ ID NO: 2122 | SEQ ID NO: 2123 |
| CD70-90 | SEQ ID NO: 2124 | SEQ ID NO: 2125 | SEQ ID NO: 2126 |
| CD70-91 | SEQ ID NO: 2127 | SEQ ID NO: 2128 | SEQ ID NO: 2129 |
| CD70-92 | SEQ ID NO: 2130 | SEQ ID NO: 2131 | SEQ ID NO: 2132 |
| CD70-93 | SEQ ID NO: 2133 | SEQ ID NO: 2134 | SEQ ID NO: 2135 |
| CD70-94 | SEQ ID NO: 2136 | SEQ ID NO: 2137 | SEQ ID NO: 2138 |
| CD70-95 | SEQ ID NO: 2139 | SEQ ID NO: 2140 | SEQ ID NO: 2141 |
| CD70-96 | SEQ ID NO: 2142 | SEQ ID NO: 2143 | SEQ ID NO: 2144 |
| CD70-97 | SEQ ID NO: 2145 | SEQ ID NO: 2146 | SEQ ID NO: 2147 |
| CD70-98 | SEQ ID NO: 2148 | SEQ ID NO: 2149 | SEQ ID NO: 2150 |
| CD70-99 | SEQ ID NO: 2151 | SEQ ID NO: 2152 | SEQ ID NO: 2153 |
| CD70-100 | SEQ ID NO: 2154 | SEQ ID NO: 2155 | SEQ ID NO: 2156 |
| CD70-101 | SEQ ID NO: 2157 | SEQ ID NO: 2158 | SEQ ID NO: 2159 |
| CD70-102 | SEQ ID NO: 2160 | SEQ ID NO: 2161 | SEQ ID NO: 2162 |
| CD70-103 | SEQ ID NO: 2163 | SEQ ID NO: 2164 | SEQ ID NO: 2165 |
| CD70-104 | SEQ ID NO: 2166 | SEQ ID NO: 2167 | SEQ ID NO: 2168 |
| CD70-105 | SEQ ID NO: 2169 | SEQ ID NO: 2170 | SEQ ID NO: 2171 |
| CD70-106 | SEQ ID NO: 2172 | SEQ ID NO: 2173 | SEQ ID NO: 2174 |
| CD70-107 | SEQ ID NO: 2175 | SEQ ID NO: 2176 | SEQ ID NO: 2177 |
| CD70-108 | SEQ ID NO: 2178 | SEQ ID NO: 2179 | SEQ ID NO: 2180 |
| CD70-109 | SEQ ID NO: 2181 | SEQ ID NO: 2182 | SEQ ID NO: 2183 |
| CD70-110 | SEQ ID NO: 2184 | SEQ ID NO: 2185 | SEQ ID NO: 2186 |
| CD70-111 | SEQ ID NO: 2187 | SEQ ID NO: 2188 | SEQ ID NO: 2189 |
| CD70-112 | SEQ ID NO: 2190 | SEQ ID NO: 2191 | SEQ ID NO: 2192 |
| CD70-113 | SEQ ID NO: 2193 | SEQ ID NO: 2194 | SEQ ID NO: 2195 |
| CD70-114 | SEQ ID NO: 2196 | SEQ ID NO: 2197 | SEQ ID NO: 2198 |
| CD70-115 | SEQ ID NO: 2199 | SEQ ID NO: 2200 | SEQ ID NO: 2201 |
| CD70-116 | SEQ ID NO: 2202 | SEQ ID NO: 2203 | SEQ ID NO: 2204 |
| CD70-117 | SEQ ID NO: 2205 | SEQ ID NO: 2206 | SEQ ID NO: 2207 |
| CD70-118 | SEQ ID NO: 2208 | SEQ ID NO: 2209 | SEQ ID NO: 2210 |
| CD70-119 | SEQ ID NO: 2211 | SEQ ID NO: 2212 | SEQ ID NO: 2213 |
| CD70-120 | SEQ ID NO: 2214 | SEQ ID NO: 2215 | SEQ ID NO: 2216 |
| CD70-121 | SEQ ID NO: 2217 | SEQ ID NO: 2218 | SEQ ID NO: 2219 |
| CD70-122 | SEQ ID NO: 2220 | SEQ ID NO: 2221 | SEQ ID NO: 2222 |
| CD70-123 | SEQ ID NO: 2223 | SEQ ID NO: 2224 | SEQ ID NO: 2225 |
| CD70-124 | SEQ ID NO: 2226 | SEQ ID NO: 2227 | SEQ ID NO: 2228 |
| CD70-125 | SEQ ID NO: 2229 | SEQ ID NO: 2230 | SEQ ID NO: 2231 |
| CD70-126 | SEQ ID NO: 2232 | SEQ ID NO: 2233 | SEQ ID NO: 2234 |
| CD70-127 | SEQ ID NO: 2235 | SEQ ID NO: 2236 | SEQ ID NO: 2237 |
| CD70-128 | SEQ ID NO: 2238 | SEQ ID NO: 2239 | SEQ ID NO: 2240 |
| CD70-129 | SEQ ID NO: 2241 | SEQ ID NO: 2242 | SEQ ID NO: 2243 |
| CD70-130 | SEQ ID NO: 2244 | SEQ ID NO: 2245 | SEQ ID NO: 2246 |
| CD70-131 | SEQ ID NO: 2247 | SEQ ID NO: 2248 | SEQ ID NO: 2249 |
| CD70-132 | SEQ ID NO: 2250 | SEQ ID NO: 2251 | SEQ ID NO: 2252 |
| CD70-133 | SEQ ID NO: 2253 | SEQ ID NO: 2254 | SEQ ID NO: 2255 |
| CD70-134 | SEQ ID NO: 2256 | SEQ ID NO: 2257 | SEQ ID NO: 2258 |
| CD70-135 | SEQ ID NO: 2259 | SEQ ID NO: 2260 | SEQ ID NO: 2261 |
| CD70-136 | SEQ ID NO: 2262 | SEQ ID NO: 2263 | SEQ ID NO: 2264 |
| CD70-137 | SEQ ID NO: 2265 | SEQ ID NO: 2266 | SEQ ID NO: 2267 |
| CD70-138 | SEQ ID NO: 2268 | SEQ ID NO: 2269 | SEQ ID NO: 2270 |
| CD70-139 | SEQ ID NO: 2271 | SEQ ID NO: 2272 | SEQ ID NO: 2273 |
| CD70-140 | SEQ ID NO: 2274 | SEQ ID NO: 2275 | SEQ ID NO: 2276 |
| CD70-141 | SEQ ID NO: 2277 | SEQ ID NO: 2278 | SEQ ID NO: 2279 |
| CD70-142 | SEQ ID NO: 2280 | SEQ ID NO: 2281 | SEQ ID NO: 2282 |
| CD70-143 | SEQ ID NO: 2283 | SEQ ID NO: 2284 | SEQ ID NO: 2285 |
| CD70-144 | SEQ ID NO: 2286 | SEQ ID NO: 2287 | SEQ ID NO: 2288 |
| CD70-145 | SEQ ID NO: 2289 | SEQ ID NO: 2290 | SEQ ID NO: 2291 |
| CD70-146 | SEQ ID NO: 2292 | SEQ ID NO: 2293 | SEQ ID NO: 2294 |
| CD70-147 | SEQ ID NO: 2295 | SEQ ID NO: 2296 | SEQ ID NO: 2297 |
| CD70-148 | SEQ ID NO: 2298 | SEQ ID NO: 2299 | SEQ ID NO: 2300 |
| CD70-149 | SEQ ID NO: 2301 | SEQ ID NO: 2302 | SEQ ID NO: 2303 |
| CD70-150 | SEQ ID NO: 2304 | SEQ ID NO: 2305 | SEQ ID NO: 2306 |
| CD70-151 | SEQ ID NO: 2307 | SEQ ID NO: 2308 | SEQ ID NO: 2309 |
| CD70-152 | SEQ ID NO: 2310 | SEQ ID NO: 2311 | SEQ ID NO: 2312 |
| CD70-153 | SEQ ID NO: 2313 | SEQ ID NO: 2314 | SEQ ID NO: 2315 |
| CD70-154 | SEQ ID NO: 2316 | SEQ ID NO: 2317 | SEQ ID NO: 2318 |
| CD70-155 | SEQ ID NO: 2319 | SEQ ID NO: 2320 | SEQ ID NO: 2321 |
| CD70-156 | SEQ ID NO: 2322 | SEQ ID NO: 2323 | SEQ ID NO: 2324 |
| CD70-157 | SEQ ID NO: 2325 | SEQ ID NO: 2326 | SEQ ID NO: 2327 |
| CD70-158 | SEQ ID NO: 2328 | SEQ ID NO: 2329 | SEQ ID NO: 2330 |
| CD70-159 | SEQ ID NO: 2331 | SEQ ID NO: 2332 | SEQ ID NO: 2333 |
| CD70-160 | SEQ ID NO: 2334 | SEQ ID NO: 2335 | SEQ ID NO: 2336 |
| CD70-161 | SEQ ID NO: 2337 | SEQ ID NO: 2338 | SEQ ID NO: 2339 |
| CD70-162 | SEQ ID NO: 2340 | SEQ ID NO: 2341 | SEQ ID NO: 2342 |
| CD70-163 | SEQ ID NO: 2343 | SEQ ID NO: 2344 | SEQ ID NO: 2345 |
| CD70-164 | SEQ ID NO: 2346 | SEQ ID NO: 2347 | SEQ ID NO: 2348 |
| CD70-165 | SEQ ID NO: 2349 | SEQ ID NO: 2350 | SEQ ID NO: 2351 |
| CD70-166 | SEQ ID NO: 2352 | SEQ ID NO: 2353 | SEQ ID NO: 2354 |
| CD70-167 | SEQ ID NO: 2355 | SEQ ID NO: 2356 | SEQ ID NO: 2357 |
| CD70-168 | SEQ ID NO: 2358 | SEQ ID NO: 2359 | SEQ ID NO: 2360 |
| CD70-169 | SEQ ID NO: 2361 | SEQ ID NO: 2362 | SEQ ID NO: 2363 |
| CD70-170 | SEQ ID NO: 2364 | SEQ ID NO: 2365 | SEQ ID NO: 2366 |
| CD70-171 | SEQ ID NO: 2367 | SEQ ID NO: 2368 | SEQ ID NO: 2369 |
| CD70-172 | SEQ ID NO: 2370 | SEQ ID NO: 2371 | SEQ ID NO: 2372 |
| CD70-173 | SEQ ID NO: 2373 | SEQ ID NO: 2374 | SEQ ID NO: 2375 |
| CD70-174 | SEQ ID NO: 2376 | SEQ ID NO: 2377 | SEQ ID NO: 2378 |
| CD70-175 | SEQ ID NO: 2379 | SEQ ID NO: 2380 | SEQ ID NO: 2381 |
| CD70-176 | SEQ ID NO: 2382 | SEQ ID NO: 2383 | SEQ ID NO: 2384 |
| CD70-177 | SEQ ID NO: 2385 | SEQ ID NO: 2386 | SEQ ID NO: 2387 |
| CD70-178 | SEQ ID NO: 2388 | SEQ ID NO: 2389 | SEQ ID NO: 2390 |
| CD70-179 | SEQ ID NO: 2391 | SEQ ID NO: 2392 | SEQ ID NO: 2393 |
| CD70-180 | SEQ ID NO: 2394 | SEQ ID NO: 2395 | SEQ ID NO: 2396 |
| 1C2 | SEQ ID NO: 2397 | SEQ ID NO: 2398 | SEQ ID NO: 2399 |
| 9D1 | SEQ ID NO: 2400 | SEQ ID NO: 2401 | SEQ ID NO: 2402 |
| 8B12 | SEQ ID NO: 2403 | SEQ ID NO: 2404 | SEQ ID NO: 2405 |
| 8C12 | SEQ ID NO: 2406 | SEQ ID NO: 2407 | SEQ ID NO: 2408 |
| 9E1 | SEQ ID NO: 2409 | SEQ ID NO: 2410 | SEQ ID NO: 2411 |
| 5F4 | SEQ ID NO: 2412 | SEQ ID NO: 2413 | SEQ ID NO: 2414 |
| 5B2 | SEQ ID NO: 2415 | SEQ ID NO: 2416 | SEQ ID NO: 2417 |
| 6D5 | SEQ ID NO: 2418 | SEQ ID NO: 2419 | SEQ ID NO: 2420 |
| 4D2 | SEQ ID NO: 2421 | SEQ ID NO: 2422 | SEQ ID NO: 2423 |
| 9A1 | SEQ ID NO: 2424 | SEQ ID NO: 2425 | SEQ ID NO: 2426 |
| 9G2 | SEQ ID NO: 2427 | SEQ ID NO: 2428 | SEQ ID NO: 2429 |
| 9B2 | SEQ ID NO: 2430 | SEQ ID NO: 2431 | SEQ ID NO: 2432 |
| 24E3 | SEQ ID NO: 2433 | SEQ ID NO: 2434 | SEQ ID NO: 2435 |
| 33D8 | SEQ ID NO: 2436 | SEQ ID NO: 2437 | SEQ ID NO: 2438 |
| 24F2 | SEQ ID NO: 2439 | SEQ ID NO: 2440 | SEQ ID NO: 2441 |
| 24B6 | SEQ ID NO: 2442 | SEQ ID NO: 2443 | SEQ ID NO: 2444 |
| 19G10 | SEQ ID NO: 2445 | SEQ ID NO: 2446 | SEQ ID NO: 2447 |
| 45B12 | SEQ ID NO: 2448 | SEQ ID NO: 2449 | SEQ ID NO: 2450 |
| 45D9 | SEQ ID NO: 2451 | SEQ ID NO: 2452 | SEQ ID NO: 2453 |
| 45F8 | SEQ ID NO: 2454 | SEQ ID NO: 2455 | SEQ ID NO: 2456 |
| 45A12 | SEQ ID NO: 2457 | SEQ ID NO: 2458 | SEQ ID NO: 2459 |
| 45B6 | SEQ ID NO: 2460 | SEQ ID NO: 2461 | SEQ ID NO: 2462 |
| 57B6 | SEQ ID NO: 2463 | SEQ ID NO: 2464 | SEQ ID NO: 2465 |
| 59D10 | SEQ ID NO: 2466 | SEQ ID NO: 2467 | SEQ ID NO: 2468 |
| 27B3 | SEQ ID NO: 2469 | SEQ ID NO: 2470 | SEQ ID NO: 2471 |
| 36A9 | SEQ ID NO: 2472 | SEQ ID NO: 2473 | SEQ ID NO: 2474 |
| 53F1 | SEQ ID NO: 2475 | SEQ ID NO: 2476 | SEQ ID NO: 2477 |
| 36D6 | SEQ ID NO: 2478 | SEQ ID NO: 2479 | SEQ ID NO: 2480 |
| 53G1 | SEQ ID NO: 2481 | SEQ ID NO: 2482 | SEQ ID NO: 2483 |
| 35G3 | SEQ ID NO: 2484 | SEQ ID NO: 2485 | SEQ ID NO: 2486 |
| 53C1 | SEQ ID NO: 2487 | SEQ ID NO: 2488 | SEQ ID NO: 2489 |
| 35F6 | SEQ ID NO: 2490 | SEQ ID NO: 2491 | SEQ ID NO: 2492 |
| 36G2 | SEQ ID NO: 2493 | SEQ ID NO: 2494 | SEQ ID NO: 2495 |
| 39D5 | SEQ ID NO: 2496 | SEQ ID NO: 2497 | SEQ ID NO: 2498 |
| 42D12 | SEQ ID NO: 2499 | SEQ ID NO: 2500 | SEQ ID NO: 2501 |
| 35C1 | SEQ ID NO: 2502 | SEQ ID NO: 2503 | SEQ ID NO: 2504 |
| 41D12 | SEQ ID NO: 2505 | SEQ ID NO: 2506 | SEQ ID NO: 2507 |
| 41H8 | SEQ ID NO: 2508 | SEQ ID NO: 2509 | SEQ ID NO: 2510 |
| 35G2 | SEQ ID NO: 2511 | SEQ ID NO: 2512 | SEQ ID NO: 2513 |
| 40F1 | SEQ ID NO: 2514 | SEQ ID NO: 2515 | SEQ ID NO: 2516 |
| 53B1 | SEQ ID NO: 2517 | SEQ ID NO: 2518 | SEQ ID NO: 2519 |
| 39C3 | SEQ ID NO: 2520 | SEQ ID NO: 2521 | SEQ ID NO: 2522 |
| 53D1 | SEQ ID NO: 2523 | SEQ ID NO: 2524 | SEQ ID NO: 2525 |
| 53H1 | SEQ ID NO: 2526 | SEQ ID NO: 2527 | SEQ ID NO: 2528 |
| 53A2 | SEQ ID NO: 2529 | SEQ ID NO: 2530 | SEQ ID NO: 2531 |
| ARGX-110 | SEQ ID NO: 2532 | SEQ ID NO: 2533 | SEQ ID NO: 2534 |
| CTX-130 | SEQ ID NO: 2535 | SEQ ID NO: 2536 | SEQ ID NO: 2537 |
| CTX-130 | SEQ ID NO: 2538 | SEQ ID NO: 2539 | SEQ ID NO: 2540 |
| 4SCAR70 | SEQ ID NO: 2541 | SEQ ID NO: 2542 | SEQ ID NO: 2543 |

C. Signal Peptides

In some embodiments, any of the CARs provided herein comprises a signal peptide (also known as a signal peptide, signal sequence, signal peptide sequence, leader peptide, and leader peptide sequence). In some embodiments, the antigen recognition domain of the CAR described herein comprises a signal peptide or a leader peptide sequence. Exemplary signal sequences include but are not limited to a CD27 signal sequence, CD8alpha signal sequence or a human IgG heavy chain signal sequence. In some embodiments, the CAR described herein does not comprise a signal peptide. In some embodiments, the NK cell or populations of NK cells provided herein comprise a CAR comprising a signal peptide. In some embodiments, the NK cell or populations of NK cell provided herein comprise a CAR that does not comprise a signal peptide.

In some embodiments, the CAR (e.g., the antigen recognition domain of the CAR) may comprise a human CD8alpha signal sequence comprising an amino acid sequence having at least 90%, 91%, 92%, 93%, 94%, 95%, 96%, 97%, 98%, 99% or 100% identity with the amino acid sequence of SEQ ID NO: 710.

In some embodiments, the CAR (e.g., the antigen recognition domain of the CAR) may comprise a human CD27 signal sequence comprising an amino acid sequence having at least 90%, 91%, 92%, 93%, 94%, 95%, 96%, 97%, 98%, 99% or 100% identity with the amino acid sequence of SEQ ID NO: 711.

In some embodiments, the CAR (e.g., the antigen recognition domain of the CAR) may comprise a human IgG heavy chain signal sequence comprising an amino acid sequence having at least 90%, 91%, 92%, 93%, 94%, 95%, 96%, 97%, 98%, 99% or 100% identity with the amino acid sequence of SEQ ID NO: 2544.

D. Hinge Domains

In some embodiments, a hinge domain (also known as a spacer region or a stalk region) is located between the antigen recognition domain and the transmembrane domain of the CAR. In particular, stalk regions are used to provide more flexibility and accessibility for the extracellular antigen recognition domain. In some embodiments, a hinge domain may comprise up to about 300 amino acids. In some embodiments, the hinge comprises about 10 to about 100 amino acids in length. In some embodiments, the hinge comprises about 25 to about 50 amino acids in length. In some embodiments, the hinge domain establishes an optimal effector-target inter membrane distance. In some embodiments, the hinge domain provides flexibility for antigen recognition domain to bind the target antigen. Any protein that is stable and/or dimerizes can serve this purpose.

A hinge domain may be derived from all or part of naturally occurring molecules, such as from all or part of the extracellular region of CD8, CD8alpha, CD4, CD28, 4-1BB, or IgG (in particular, the hinge domain of an IgG, for example from IgG1, IgG2, IgG3, or IgG4), or from all or part of an antibody heavy-chain constant region. Alternatively, the hinge domain may be a synthetic sequence that corresponds to a naturally occurring hinge sequence, or may be an entirely synthetic hinge sequence. In some embodiments, it corresponds to Fc domains of a human immunoglobulin, e.g., either the CH2 or CH3 domain. In some embodiments, the CH2 and CH3 hinge domains of a human immunoglobulin that has been modified to improve dimerization. In some embodiments, the hinge is a hinge portion of an immunoglobulin. In some embodiments, the hinge domain comprises a CH3 region of a human immunoglobulin. In some embodiments, the hinge domain comprises a CH2 and CH3 region of a human immunoglobulin. In some embodiments, the CH2 region comprises a human IgG1, IgG2 or IgG4 immunoglobulin CH2 region. In some embodiments, the hinge domain is from an IgG (e.g., IgG1, IgG2, IgG3 or IgG4) and the domain comprises one or more mutations (e.g., amino acid substitutions (e.g., in its CH2 domain) so as to prevent or reduce off-target binding of the hinge domain and/or a CAR comprising the hinge domain to an Fc receptor. In some embodiments, the hinge domain is derived from an IgG1, IgG2, IgG3, or IgG4 Fc region and includes one or more amino acid substitutions as compared to the wild-type protein from which the hinge domain was derived. In some embodiments, the hinge domain is derived from an IgG1, IgG2, IgG3, or IgG4 Fc region and includes one or more (e.g., two, three, four, five, six, seven, eight, nine, ten, eleven, twelve, thirteen, fourteen, fifteen, twenty, twenty-five, thirty, or more) amino acid substitutions at an amino acid residue at position 220, 226, 228, 229, 230, 233, 234, 235, 234, 237, 238, 239, 243, 247, 267, 268, 280, 290, 292, 297, 298, 299, 300, 305, 309, 318, 326, 330, 331, 332, 333, 334, 336, and/or 339 (amino acid residue positions indicated in the EU index proposed in Kabat et al. (1991) Sequences of Proteins of Immunological Interest, 5th Ed., United States Public Health Service, National Institutes of Health, Bethesda). In some embodiments, the hinge domain is derived from an IgG1, IgG2, IgG3, or IgG4 Fc region and includes one or more (e.g., two, three, four, five, six, seven, eight, nine, ten, eleven, twelve, thirteen, fourteen, fifteen, twenty, twenty-five, thirty, or more) of the following amino acid substitutions C220S, C226S, S228P, C229S, P230S, E233P, V234A, L234V, L234F, L234A, L235A, L235E, G236A, G237A, P238S, S239D, F243L, P247I, S267E, H268Q, S280H, K290S, K290E, K290N, R292P, N297A, N297Q, S298A, S298G, S298D, S298V, T299A, Y300L, V3051, V309L, E318A, K326A, K326W, K326E, L328F, A330L, A330S, A331S, P331S, 1332E, E333A, E333S, E333S, K334A, A339D, A339Q, and P396L. In some embodiments, the hinge domain is derived from an IgG1, IgG2, IgG3, or IgG4 Fc region and includes one or more of the following combinations of amino acid substitutions: S228P and L235E; S228P and N297Q; L235E and N297Q; S228P, L235E, and N297Q.

In some embodiments, the hinge domain is a part of human CD8α chain (e.g., NP_001139345.1). In some embodiments, the hinge domain of CARs described herein comprises a subsequence of CD8a, an IgG1, an IgG4, FcγRIIIα or CD28, in particular the hinge domain of any of a CD8a, an IgG1, an IgG4, FcγRIIIα or a CD28. In some embodiments, the stalk region comprises a human CD8α hinge, a human IgG1 hinge, a human IgG4 hinge, a human FcγRIIIα hinge, or a human CD28 hinge.

Any of the CARs provided herein may comprise a hinge domain described herein. In some embodiments, the hinge may comprise or consist of a human CD8alpha hinge domain comprising an amino acid sequence having at least 90%, 91%, 92%, 93%, 94%, 95%, 96%, 97%, 98%, 99% or 100% identity with the amino acid sequence of SEQ ID NO: 619. In some embodiments, the hinge may comprise or consist of a human CD8alpha hinge domain comprising an amino acid sequence having at least 90%, 91%, 92%, 93%, 94%, 95%, 96%, 97%, 98%, 99% or 100% identity with the amino acid sequence of SEQ ID NO: 2545. In some embodiments, the hinge may comprise or consist of a human IgG1 hinge domain comprising an amino acid sequence having at least 90%, 91%, 92%, 93%, 94%, 95%, 96%, 97%, 98%, 99% or 100% identity with the amino acid sequence of SEQ ID NO: 620. In some embodiments, the hinge may comprise or consist of a human IgG1 hinge domain comprising an amino acid sequence having at least 90%, 91%, 92%, 93%, 94%, 95%, 96%, 97%, 98%, 99% or 100% identity with the amino acid sequence of SEQ ID NO: 2546. In some embodiments, the hinge may comprise or consist of a human IgG4 hinge domain comprising an amino acid sequence having at least 90%, 91%, 92%, 93%, 94%, 95%, 96%, 97%, 98%, 99% or 100% identity with the amino acid sequence of SEQ ID NO: 696. In some embodiments, the hinge may comprise or consist of a human FcγRIIIα hinge domain comprising an amino acid sequence having at least 90%, 91%, 92%, 93%, 94%, 95%, 96%, 97%, 98%, 99% or 100% identity with the amino acid sequence of SEQ ID NO: 621. In some embodiments, the hinge may comprise or consist of a human CD28 hinge domain comprising an amino acid sequence having at least 90%, 91%, 92%, 93%, 94%, 95%, 96%, 97%, 98%, 99% or 100% identity with the amino acid sequence of SEQ ID NO: 2547. In some embodiments, the hinge may comprise or consist of an amino acid sequence having at least 90%, 91%, 92%, 93%, 94%, 95%, 96%, 97%, 98%, 99% or 100% identity to the amino acid sequence of any one of SEQ ID NOs: 2689-2694.

E. Transmembrane Domains

Suitable transmembrane domains for a CAR disclosed herein have the ability to (a) be expressed at the surface of a cell, which is in some embodiments an immune cell such as, for example a NK cell, and/or (b) interact with the ligand-binding domain and intracellular signaling domain for directing cellular response of an immune cell against a predefined target cell. The transmembrane domain can be derived either from a natural or from a synthetic source. The transmembrane domain can be derived from any membrane-bound or transmembrane protein. As non-limiting examples, the transmembrane domains can include the transmembrane region(s) of alpha, beta or zeta chain of the T-cell receptor; or a transmembrane region from CD8, CD8alpha, CD28, 2B4, NKG2D, CD16, CD3 zeta, CD3 epsilon, CD3 gamma, CD3 delta, CD45, CD4, CD5, CD9, CD22, CD27, CD28, CD33, CD37, CD64, CD80, CD86, CD134, CD137, CD154, ICOS/CD278, GITR/CD357, NKp44, NKp46, NKp30, DNAM-1, NKG2D, DAP, DAP10, DAP12 or erythropoietin receptor transmembrane domain or a portion of any of the foregoing or a combination of any of the foregoing. In some embodiments, the transmembrane domain comprises CD8alpha, CD16, CD28, 2B4, NKG2D, NKp44, NKp46, CD27, DAP10 or DAP12. In some embodiments, the transmembrane domain comprises a human CD8alpha transmembrane domain. In some embodiments, the transmembrane domain comprises a human CD16 transmembrane domain. In some embodiments, the transmembrane domain comprises a human CD28 transmembrane domain. In some embodiments, the transmembrane domain comprises a human NKG2D transmembrane domain. In some embodiments, the transmembrane domain comprises a human NKp44 transmembrane domain. In some embodiments, the transmembrane domain comprises a human NKp46 transmembrane domain. In some embodiments, the transmembrane domain comprises a human CD27 transmembrane domain. In some embodiments, the transmembrane domain comprises a human DAP10 transmembrane domain. In some embodiments, the transmembrane domain comprises a human DAP12 transmembrane domain.

Alternatively, the transmembrane domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the intracellular domain of a CAR. In some embodiments, the linker is a glycine-serine linker.

In some embodiments, the transmembrane domain of a CAR provided herein may comprise or consist of a human CD8alpha transmembrane domain comprising an amino acid sequence having at least 90%, 91%, 92%, 93%, 94%, 95%, 96%, 97%, 98%, 99% or 100% identity with the amino acid sequence of SEQ ID NO: 624.

In some embodiments, the transmembrane domain of a CAR provided herein may comprise or consist of a human CD8alpha transmembrane domain comprising an amino acid sequence having at least 90%, 91%, 92%, 93%, 94%, 95%, 96%, 97%, 98%, 99% or 100% identity with the amino acid sequence of SEQ ID NO: 2548.

In some embodiments, the transmembrane domain of a CAR provided herein may comprise or consist of a human CD28 transmembrane domain comprising an amino acid sequence having at least 90%, 91%, 92%, 93%, 94%, 95%, 96%, 97%, 98%, 99% or 100% identity with the amino acid sequence of SEQ ID NO: 625.

In some embodiments, the transmembrane domain of a CAR provided herein may comprise or consist of a human NKG2D transmembrane domain comprising an amino acid sequence having at least 90%, 91%, 92%, 93%, 94%, 95%, 96%, 97%, 98%, 99% or 100% identity with the amino acid sequence of SEQ ID NO: 626.

In some embodiments, the transmembrane domain of a CAR provided herein may comprise or consist of a human NKG2D transmembrane domain comprising an amino acid sequence having at least 90%, 91%, 92%, 93%, 94%, 95%, 96%, 97%, 98%, 99% or 100% identity with the amino acid sequence of SEQ ID NO: 2549.

In some embodiments, the transmembrane domain of a CAR provided herein may comprise or consist of a human CD16 transmembrane domain comprising an amino acid sequence having at least 90%, 91%, 92%, 93%, 94%, 95%, 96%, 97%, 98%, 99% or 100% identity with the amino acid sequence of SEQ ID NO: 627.

In some embodiments, the transmembrane domain of a CAR provided herein may comprise or consist of a human NKp44 transmembrane domain comprising an amino acid sequence having at least 90%, 91%, 92%, 93%, 94%, 95%, 96%, 97%, 98%, 99% or 100% identity with the amino acid sequence of SEQ ID NO: 697.

In some embodiments, the transmembrane domain of a CAR provided herein may comprise or consist of a human NKp46 transmembrane domain comprising an amino acid sequence having at least 90%, 91%, 92%, 93%, 94%, 95%, 96%, 97%, 98%, 99% or 100% identity with the amino acid sequence of SEQ ID NO: 698.

In some embodiments, the transmembrane domain of a CAR provided herein may comprise or consist of a human CD27 transmembrane domain comprising an amino acid sequence having at least 90%, 91%, 92%, 93%, 94%, 95%, 96%, 97%, 98%, 99% or 100% identity with the amino acid sequence of SEQ ID NO: 2550.

In some embodiments, the transmembrane domain of a CAR provided herein may comprise or consist of a human CD27 transmembrane domain comprising an amino acid sequence having at least 90%, 91%, 92%, 93%, 94%, 95%, 96%, 97%, 98%, 99% or 100% identity with the amino acid sequence of SEQ ID NO: 2551.

In some embodiments, the transmembrane domain of a CAR provided herein may comprise or consist of a human DAP12 transmembrane domain comprising an amino acid sequence having at least 90%, 91%, 92%, 93%, 94%, 95%, 96%, 97%, 98%, 99% or 100% identity with the amino acid sequence of SEQ ID NO: 2552.

In some embodiments, the transmembrane domain of a CAR provided herein may comprise or consist of a human DAP10 transmembrane domain comprising an amino acid sequence having at least 90%, 91%, 92%, 93%, 94%, 95%, 96%, 97%, 98%, 99% or 100% identity with the amino acid sequence of SEQ ID NO: 2553.

F. Costimulatory Domains

The intracellular domain of a CAR provided herein may comprise one or more costimulatory domains. Exemplary costimulatory domains include, but are not limited to a CD27, CD28, 4-IBB (CD137), ICOS, DAP10, DAP12, 2B4, OX40 (CD134), and OX40L costimulatory domain, or a fragment thereof, or a combination thereof. In some instances, a CAR described herein comprises one or more, or two or more of costimulatory domains selected from a CD27, CD28, 4-IBB (CD137), ICOS, DAP10, DAP12, 2B4, OX40 (CD134), and OX40L costimulatory domain, or a fragment thereof, or a combination thereof. In some embodiments, a CAR described herein comprises a CD28 costimulatory domain or a fragment thereof. In some embodiments, a CAR described herein comprises a 4-1BB (CD137) costimulatory domain or a fragment thereof. In some embodiments, a CAR described herein comprises a DAP10 costimulatory domain or a fragment thereof. In some embodiments, a CAR described herein comprises a DAP12 costimulatory domain or a fragment thereof. In some embodiments, a CAR described herein comprises a 2B4 costimulatory domain or a fragment thereof. In some embodiments, a CAR described herein comprises a OX40 costimulatory domain or a fragment thereof. In some embodiments, a CAR described herein comprises a OX40L costimulatory domain or a fragment thereof. In some embodiments, a CAR described herein comprises a ICOS costimulatory domain or a fragment thereof. In some embodiments, a CAR described herein comprises a CD27 costimulatory domain or fragment thereof.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 628.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 699.

In some embodiments, the costimulatory domain of a CAR provided herein may comprise or consist of a human 4-1BB costimulatory domain comprising an amino acid sequence having at least 90%, 91%, 92%, 93%, 94%, 95%, 96%, 97%, 98%, 99% or 100% identity with the amino acid sequence of SEQ ID NO: 629.

In some embodiments, the costimulatory domain of a CAR provided herein may comprise or consist of a human 4-1BB costimulatory domain comprising an amino acid sequence having at least 90%, 91%, 92%, 93%, 94%, 95%, 96%, 97%, 98%, 99% or 100% identity with the amino acid sequence of SEQ ID NO: 2554.

In some embodiments, the costimulatory domain of a CAR provided herein may comprise or consist of a human DAP10 costimulatory domain comprising an amino acid sequence having at least 90%, 91%, 92%, 93%, 94%, 95%, 96%, 97%, 98%, 99% or 100% identity with the amino acid sequence of SEQ ID NO: 630.

In some embodiments, the costimulatory domain of a CAR provided herein may comprise or consist of a human DAP10 costimulatory domain comprising an amino acid sequence having at least 90%, 91%, 92%, 93%, 94%, 95%, 96%, 97%, 98%, 99% or 100% identity with the amino acid sequence of SEQ ID NO: 2555.

In some embodiments, the costimulatory domain of a CAR provided herein may comprise or consist of a human DAP12 costimulatory domain comprising an amino acid sequence having at least 90%, 91%, 92%, 93%, 94%, 95%, 96%, 97%, 98%, 99% or 100% identity with the amino acid sequence of SEQ ID NO: 631.

In some embodiments, the costimulatory domain of a CAR provided herein may comprise or consist of a human 2B4 costimulatory domain comprising an amino acid sequence having at least 90%, 91%, 92%, 93%, 94%, 95%, 96%, 97%, 98%, 99% or 100% identity with the amino acid sequence of SEQ ID NO: 632.

In some embodiments, the costimulatory domain of a CAR provided herein may comprise or consist of a human OX40 costimulatory domain comprising an amino acid sequence having at least 90%, 91%, 92%, 93%, 94%, 95%, 96%, 97%, 98%, 99% or 100% identity with the amino acid sequence of SEQ ID NO: 2556.

In some embodiments, the costimulatory domain of a CAR provided herein may comprise or consist of a human OX40L costimulatory domain comprising an amino acid sequence having at least 90%, 91%, 92%, 93%, 94%, 95%, 96%, 97%, 98%, 99% or 100% identity with the amino acid sequence of SEQ ID NO: 2695.

In some embodiments, the costimulatory domain of a CAR provided herein may comprise or consist of a human CD27 costimulatory domain comprising an amino acid sequence having at least 90%, 91%, 92%, 93%, 94%, 95%, 96%, 97%, 98%, 99% or 100% identity with the amino acid sequence of SEQ ID NO: 2557.

In some embodiments, the costimulatory domain of a CAR provided herein may comprise or consist of a human CD27 costimulatory domain comprising an amino acid sequence having at least 90%, 91%, 92%, 93%, 94%, 95%, 96%, 97%, 98%, 99% or 100% identity with the amino acid sequence of SEQ ID NO: 2558.

G. Activation Domains

In some embodiments, the activation domain of a CAR disclosed herein is responsible for activation of at least one of the normal effector functions of the immune cell (e.g., NK cell) in which the CAR is expressed. The terms “intracellular signaling domain” or “intracellular domain” are used interchangeably and refer to a domain that comprises a co-stimulatory domain and/or an activation domain. The term “effector function” refers to a specialized function of a cell. Effector function of a T cell, for example, may be cytolytic activity or helper activity including the secretion of cytokines. The term “activation domain” refers to the portion of a protein which transduces the effector function signal and directs the cell to perform a specialized function. While usually an entire activation domain can be employed, in many cases it is not necessary to use the entire chain. To the extent that a truncated portion of the activation domain is used, such truncated portion may be used in place of the intact chain as long as it transduces the effector function signal. The term activation domain is thus meant to include any truncated portion of the activation domain sufficient to transduce the effector function signal. In some embodiments, the activation domain further comprises a signaling domain for T-cell activation and/or a signaling domain for NK cell activation. In some instances, the signaling domain for NK cell activation and/or T-cell activation comprises a domain derived from DAP12, TCR zeta, FcR gamma, FcR beta, FCER1G, FCGR2A, CD3 gamma, CD3 delta, CD3 epsilon, CD5, CD22, CD79a, CD79b or CD66d. In some embodiments, the CAR described herein comprises at least one (e.g., one, two, three, or more) activation domain selected from a DAP12, TCR zeta, FcR gamma, FcR beta, FCER1G, FCGR2A, CD3 gamma, CD3 delta, CD3 epsilon, CD5, CD22, CD79a, CD79b, CD66d activation domain, or a portion of any of the foregoing. In some embodiments, the CAR described herein has an activation domain comprising a domain derived from CD3 (CD3zeta). In some embodiments, the CAR described herein has an activation domain comprising a domain derived from FCER1G.

In some embodiments, the activation domain of a CAR described herein may comprise or consist of a CD3zeta activation domain (e.g., a human CD3zeta activation domain) comprising an amino acid sequence having at least 90%, 91%, 92%, 93%, 94%, 95%, 96%, 97%, 98%, 99% or 100% identity with the amino acid sequence of SEQ ID NO: 635. In some embodiments, the CD3zeta activation domain comprises a mutation in an ITAM domain. Examples of mutations in ITAM domains of CD3zeta are provided in Feucht et al., Nat Med. 2019; 25(1): 82-88. In some embodiments, each of the two tyrosine residues in one or more of ITAM1, ITAM2, or ITAM3 domains of the CD3zeta activation domain are point-mutated to a phenylalanine residue. In some embodiments, the CD3zeta activation domain comprises a deletion of one or more of the ITAM1, ITAM2, or ITAM3 domains.

In some embodiments, the activation domain of a CAR provided herein may comprise or consist of a human CD3zeta intracellular signaling domain comprising an amino acid sequence having at least 90%, 91%, 92%, 93%, 94%, 95%, 96%, 97%, 98%, 99% or 100% identity with the amino acid sequence of SEQ ID NO: 2559.

In some embodiments, the activation domain of a CAR provided herein may comprise or consist of a human FCER1G intracellular signaling domain comprising an amino acid sequence having at least 90%, 91%, 92%, 93%, 94%, 95%, 96%, 97%, 98%, 99% or 100% identity with the amino acid sequence of SEQ ID NO: 2560.

Included in the scope of the disclosure are nucleic acid sequences that encode functional portions of the CAR described herein. Functional portions encompass, for example, those parts of a CAR that retain the ability to recognize target cells, or detect, treat, or prevent a disease, to a similar extent, the same extent, or to a higher extent, as the parent CAR.

In embodiments, the CAR contains additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

A CAR described herein include (including functional portions and functional variants thereof) glycosylated, amidated, carboxylated, phosphorylated, esterified, N-acylated, cyclized via, e.g., a disulfide bridge, or converted into an acid addition salt and/or optionally dimerized or polymerized.

Table 4 provides exemplary amino acid sequences of the domains which can be used in the CARs described herein. In some embodiments, a CAR provided herein comprises one or more domains described in Table 4, or a fragment or portion thereof.

TABLE 4
Exemplary Amino Acid Sequences of CAR Domains

| | | |
|---|---|---|
| | | SEQ ID |
| Exemplary CAR domains | Amino Acid Sequence | NO: |

SIGNAL PEPTIDE

| | | |
|---|---|---|
| human CD8α signal sequence | MALPVTALLLPLALLLHAARP | 710 |
| human CD27 signal sequence | MARPHPWWLCVLGTLVGLS | 711 |
| human IgG heavy chain signal | MEFGLSWLFLVAILKGVQCSR | 2544 |
| sequence | | |

HINGES

| | | |
|---|---|---|
| human CD8α hinge domain | TTTPAPRPPTPAPTIASQPLSLRPEACRPAAGGAVHTRGLD | 619 |
| | FACD | |
| human CD8α hinge domain | FVPVFLPAKPTTTPAPRPPTPAPTIASQPLSLRPEACRPAA | 2545 |
| | GGAVHTRGLDFACD | |
| human IgG1 hinge domain | EPKSCDKTHTCPPCPAPELLGGPSVFLFPPKPKDTLMISRT | 620 |
| | PEVTCVVVDVSHEDPEVKFNWYVDGVEVHNAKTKPREEQYN | |
| | STYRVVSVLTVLHQDWLNGKEYKCKVSNKALPAPIEKTISK | |
| | AKGQPREPQVYTLPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| human IgG1 hinge domain | EPKSCDKTHTCPPCPAPELLGGPSVFLFPPKPKDTLMISRT | 2546 |
| | PEVTCVVVDVSHEDPEVKFNWYVDGVEVHNAKTKPREEQYN | |
| | STYRVVSVLTVLHQDWLNGKEYKCKVSNKALPAPIEKTISK | |
| | AKGQPREPQVYTLPPSRDELTKNQVSLTCLVKGFYPSDIAV | |
| | EWESNGQPENNYKTTPPVLDSDGSFFLYSKLTVDKSRWQQG | |
| | NVFSCSVMHEALHNHYTQKSLSLSPGK | |
| human IgG4 hinge domain | ESKYGPPCPSCPAPEFLGGPSVFLFPPKPKDTLMISRTPEV | 696 |
| | TCVVVDVSQEDPEVQFNWYVDGVEVHNAKTKPREEQFNSTY | |
| | RVVSVLTVLHQDWLNGKEYKCKVSNKGLPSSIEKTISKAKG | |
| | QPREPQVYTLPPSQEEMTKNQVSLTCLVKGFYPSDIAVEWE | |
| | SNGQPENNYKTTPPVLDSDGSFFLYSRLTVDKSRWQEGNVF | |
| | SCSVMHEALHNHYTQKSLSLSLGK | |
| human FcγRIIIα hinge | GLAVSTISSFFPPGYQ | 621 |
| domain | | |
| CD28 hinge domain | IEVMYPPPYLDNEKSNGTIIHVKGKHLCPSPLFPGPS | 2547 |
| | KP | |
| IgG1 short hinge domain | AEPKSPDKTHTCPPCPKDP | 2689 |
| IgG4 short hinge domain | ESKYGPPCPSCP | 2690 |
| IgG4 hinge-CH3 | ESKYGPPCPSCPGQPREPQVYTLPPSQEEMTKNQVSLTCLV | 2691 |
| | KGFYPSDIAVEWESNGQPENNYKTTPPVLDSDGSFFLYSRL | |
| | TVDKSRWQEGNVFSCSVMHEALHNHYTQKSLSLSLGK | |
| IgG4 mutant hinge domain | ESKYGPPCPSCPAPEFEGGPSVFLFPPKPKDTLMISRTPEV | 2962 |
| | TCVVVDVSQEDPEVQFNWYVDGVEVHNAKTKPREEQFQSTY | |
| | RVVSVLTVLHQDWLNGKEYKCKVSNKGLPSSIEKTISKAKG | |
| | QPREPQVYTLPPSQEEMTKNQVSLTCLVKGFYPSDIAVEWE | |
| | SNGQPENNYKTTPPVLDSDGSFFLYSRLTVDKSRWQEGNVF | |
| | SCSVMHEALHNHYTQKSLSLSLGK | |
| IgG4 mutant-1 hinge domain | ESKYGPPCPSCPAPEFEGGPSVFLFPPKPKDTLMISRTPEV | 2693 |
| | TCVVVDVSQEDPEVQFNWYVDGVEVHNAKTKPREEQFNSTY | |
| | RVVSVLTVLHQDWLNGKEYKCKVSNKGLPSSIEKTISKAKG | |
| | QPREPQVYTLPPSQEEMTKNQVSLTCLVKGFYPSDIAVEWE | |
| | SNGQPENNYKTTPPVLDSDGSFFLYSRLTVDKSRWQEGNVF | |
| | SCSVMHEALHNHYTQKSLSLSLGK | |
| IgG4 mutant-2 hinge domain | ESKYGPPCPSCPAPEFLGGPSVFLFPPKPKDTLMISRTPEV | 2694 |
| | TCVVVDVSQEDPEVQFNWYVDGVEVHNAKTKPREEQFQSTY | |
| | RVVSVLTVLHQDWLNGKEYKCKVSNKGLPSSIEKTISKAKG | |
| | QPREPQVYTLPPSQEEMTKNQVSLTCLVKGFYPSDIAVEWE | |
| | SNGQPENNYKTTPPVLDSDGSFFLYSRLTVDKSRWQEGNVF | |
| | SCSVMHEALHNHYTQKSLSLSLGK | |

TRANSMEMBRANE DOMAINS

| | | |
|---|---|---|
| human CD8α transmembrane | IYIWAPLAGTCGVLLLSLVIT | 624 |
| domain | | |
| human CD8α transmembrane | IYIWAPLAGTCGVLLLSLVITLYCNHRN | 2548 |
| domain | | |
| human CD28 transmembrane | FWVLVVVGGVLACYSLLVTVAFIIFWV | 625 |
| domain | | |
| human NKG2D | VVRVLAIALAIRFTLNTLMWLAI | 626 |
| transmembrane domain | | |
| human NKG2D | PFFFCCFIAVAMGIRFIIMVAIWSAVFLNS | 2549 |
| transmembrane domain | | |
| human CD16 transmembrane | VSFCLVMVLLFAVDTGLYFSV | 627 |
| domain | | |
| human NKp44 | LVPVFCGLLVAKSLVLSALLV | 697 |
| transmembrane domain | | |
| human NKp46 | MGLAFLVLVALVWFLVEDWLS | 698 |
| transmembrane domain | | |
| human CD27 transmembrane | ILVIFSGMFLVFTLAGALFL | 2550 |
| domain | | |
| human CD27 transmembrane | ILVIFSGMFLVFTLAGALFLH | 2551 |
| domain | | |
| human DAP12 | GVLAGIVMGDLVLTVLIALAV | 2552 |
| transmembrane domain | | |
| human DAP10 | LLAGLVAADAVASLLIVGAVF | 2553 |
| transmembrane domain | | |

COSTIMULATORY DOMAINS

| | | |
|---|---|---|
| human CD28 costimulatory | RSKRSRGGHSDYMNMTPRRPGPTRKHYQPYAPPRDFAAYRS | 628 |
| domain | | |
| human CD28 costimulatory | RSKRSRLLHSDYMNMTPRRPGPTRKHYQPYAPPRDFAAYRS | 699 |
| domain | | |
| human 4-1BB costimulatory | KRGRKKLLYIFKQPFMRPVQTTQEEDGCSCRFPEEEEGGCE | 629 |
| domain | L | |
| human 4-1BB costimulatory | RKRGRKKLLYIFKQPFMRPVQTTQEEDGCSCRFPEEEEGGC | 2554 |
| domain | EL | |
| human DAP10 costimulatory | LCARPRRSPAQEDGKVYINMPGRG | 630 |
| domain | | |
| human DAP10 costimulatory | CARPRRSPAQEDGKVYINMPGRG | 2555 |
| domain | | |
| human DAP12 costimulatory | YFLGRLVPRGRGAAEAATRKQRITETESPYQELQGQRSDVY | 631 |
| domain | SDLNTQRPYYK | |
| human 2B4 costimulatory | WRRKRKEKQSETSPKEFLTIYEDVKDLKTRRNHEQEQTFPG | 632 |
| domain | GGSTIYSMIQSQSSAPTSQEPAYTLYSLIQPSRKSGSRKRN | |
| | HSPSFNSTIYEVIGKSQPKAQNPARLSRKELENFDVYS | |
| human OX40 costimulatory | ALYLLRRDQRLPPDAHKPPGGGSFRTPIQEEQADAHSTLAK | 2556 |
| domain | I | |
| human CD27 costimulatory | HQRRKYRSNKGESPVEPAEPCHYSCPREEEGSTIPIQEDYR | 2557 |
| domain | KPEPACSP | |
| human CD27 costimulatory | QRRKYRSNKGESPVEPAEPCHYSCPREEEGSTIPIQEDYRK | 2558 |
| domain | PEPACSP | |
| human OX40L costimulatory | ERVQPLEENVGNAARPRFERNK | 2695 |
| domain | | |

ACTIVATION DOMAINS

| | | |
|---|---|---|
| human CD3zeta intracellular | RVKFSRSADAPAYKQGQNQLYNELNLGRREEYDVLDKRRGR | 635 |
| signaling domain | DPEMGGKPRRKNPQEGLYNELQKDKMAEAYSEIGMKGERRR | |
| | GKGHDGLYQGLSTATKDTYDALHMQALPPR | |
| human CD3zeta intracellular | RVKFSRSADAPAYQQGQNQLYNELNLGRREEYDVLDKRRGR | 2559 |
| signaling domain | DPEMGGKPRRKNPQEGLYNELQKDKMAEAYSEIGMKGERRR | |
| | GKGHDGLYQGLSTATKDTYDALHMQALPPR | |
| human FCER1G intracellular | RLKIQVRKAAITSYEKSDGVYTGLSTRNQETYETLKHEKPP | 2560 |
| signaling domain | Q | |
| human FCGR2A | CRKKRISANSTDPVKAAQFEPPGRQMIAIRKRQLEETNNDY | 2642 |
| intracellular signaling | ETADGGYMTLNPRAPTDDDKNIYLTLPPNDHVNSNN | |
| domain | | |

H. Exemplary Anti-CD70 CAR Constructs

Disclosed herein are a chimeric antigen receptor (CAR), wherein the CAR comprises (a) an antigen recognition domain that specifically binds human CD70; (b) a hinge domain comprising or consisting of a CD8α (e.g., a human CD8α hinge domain), IgG1 (e.g., an IgG1 hinge domain or IgG1 short hinge domain), IgG4 (e.g., an IgG4 hinge domain, an IgG4 short hinge domain, an IgG4 hinge-CH3, IgG4 mutant hinge domain, an IgG4 mutant-1 hinge domain, or an IgG4 mutant-2 hinge domain) or CD28 hinge domain; (c) a transmembrane domain comprising or consisting of a CD16, CD27, CD28, CD8a (e.g., a, DAP10, DAP12, NKp44, NKp46, or NKG2D transmembrane domain; (d) a costimulatory domain comprising or consisting of a CD28, DAP10, DAP12, CD27, 4-1BB, 2B4, OX40 or OX40L costimulatory domain; optionally (e), a costimulatory signaling domain comprising or consisting of a CD28, DAP10, DAP12, CD27, 4-1BB, 2B4, OX40 or OX40L costimulatory domain; and optionally (f), an activation domain comprising or consisting of a CD3zeta or FCER1G activation domain. Also disclosed herein are nucleic acid sequences encoding said CARs and immune cells comprising said nucleic acids.

Table 5 provides exemplary anti-CD70 CAR constructs disclosed herein and the domains that they comprise. In some embodiments, an immune cell (e.g., an NK cell) or a population of immune cells (e.g., NK cells) described herein is genetically modified to express at least one of the exemplary anti-CD70 CAR constructs provided in Table 5. In some embodiments, an immune cell (e.g., an NK cell) or a population of immune cells (e.g., NK cells) comprises one of the exemplary anti-CD70 CAR constructs provided in Table 5.

TABLE 5
Exemplary anti-CD70 CAR constructs and domains

| | Signal | Antigen Recognition | | Transmembrane | Intracelllular | Intracellular | Intracellular |
|---|---|---|---|---|---|---|---|
| ID | Peptide (SP) | Domain (Binder) | Hinge Domain | (TM) Domain | Domain 1 | Domain 2 | Domain 3 |
| CAT-70-001 | CD8α SP | CD70 scFv (1F6) | CD8α hinge | CD28 | CD28 | CD3z | — |
| CAT-70-002 | CD8α SP | CD70 scFv (1F6) | CD8α hinge | NKG2D | DAP10 | CD3z | — |
| CAT-70-003 | CD8α SP | CD70 scFv (1F6) | CD8α hinge | NKG2D | DAP12 | CD3z | — |
| CAT-70-004 | CD27 SP | CD27 | — | CD27 | CD27 | CD3z | — |
| (Construct #1) | | extracellular | | | | | |
| | | domain (ECD) | | | | | |
| CAT-70-005 | CD27 SP | CD27 ECD | — | CD28 | CD28 | CD3z | — |
| CAT-70-006 | CD27 SP | CD27 ECD | — | NKG2D | DAP10 | CD3z | — |
| CAT-70-007 | CD27 SP | CD27 ECD | — | NKG2D | DAP12 | CD3z | — |
| CAT-CD70-119 | CD27 SP | CD27 ECD | — | CD27 | 4-1BB | CD3z | — |
| CAT-CD70-122 | CD27 SP | CD27 ECD | CD8α hinge | CD8a | 4-1BB | CD3z | — |
| CAT-CD70-124 | CD27 SP | CD27 ECD | — | CD27 | CD28 | CD3z | — |
| CAT-CD70-125 | CD27 SP | CD27 ECD | CD8α hinge | CD8a | CD28 | CD3z | — |
| CAT-CD70-127 | CD8α SP | CD70 scFv (1F6) | CD8α hinge | CD8a | 4-1BB | CD3z | — |
| (Construct #2) | | | | | | | |
| CAT-CD70-130 | CD8α SP | CD70 scFv (1F6) | IgG1 hinge | CD28 | CD28 | CD3z | — |
| CAT-CD70-133 | CD8α SP | CD70 scFv (1F6) | CD28 hinge | CD28 | CD28 | CD3z | — |
| CAT-CD70-135 | CD8α SP | CD70 scFv (1F6) | CD8α hinge | CD8a | CD28 | CD3z | — |
| CAT-CD70-136 | CD27 SP | CD27 ECD | — | CD27 | CD27 | DAP12 | — |
| CAT-CD70-137 | CD27 SP | CD27 ECD | — | CD27 | CD27 | FCER1G | — |
| CAT-CD70-140 | CD27 SP | CD27 ECD | — | DAP10 | DAP10 | CD3z | — |
| CAT-CD70-141 | CD27 SP | CD27 ECD | — | DAP12 | DAP12 | CD3z | — |
| CAT-CD70-142 | CD27 SP | CD27 ECD | — | DAP12 | DAP12 | — | — |
| CAT-CD70-143 | CD8α SP | CD70 scFv (1F6) | IgG1 hinge | CD28 | CD28 | — | — |
| CAT-CD70-144 | CD8α SP | CD70 scFv (1F6) | CD8α hinge | CD8a | 4-1BB | — | — |
| CAT-CD70-145 | CD8α SP | CD70 scFv (1F6) | CD8α hinge | CD8a | — | CD3z | — |
| CAT-CD70-146 | CD8α SP | CD70 scFv (1F6) | CD8α hinge | CD8a | 4-1BB | 4-1BB | — |
| CAT-CD70-147 | CD8α SP | CD70 scFv (1F6) | CD8α hinge | CD8a | 2B4 | CD3z | — |
| CAT-CD70-148 | CD8α SP | CD70 scFv (1F6) | CD8α hinge | CD8a | DAP10 | CD3z | — |
| CAT-CD70-149 | CD8α SP | CD70 scFv (1F6) | CD8α hinge | CD8a | DAP12 | CD3z | — |
| CAT-CD70-150 | CD8α SP | CD70 scFv (1F6) | CD8α hinge | CD8a | OX40 | CD3z | — |
| CAT-CD70-153 | CD8α SP | CD70 scFv (1F6) | CD8α hinge | NKG2D | 2B4 | CD3z | — |
| CAT-CD70-154 | CD8α SP | CD70 scFv (1F6) | CD8α hinge | DAP10 | DAP10 | CD3z | — |
| CAT-CD70-155 | CD8α SP | CD70 scFv (1F6) | CD8α hinge | DAP12 | DAP12 | CD3z | — |
| CAT-CD70-156 | CD8α SP | CD70 scFv (1F6) | CD8α hinge | DAP12 | DAP12 | — | — |
| CAT-CD70-157 | CD8α SP | CD70 scFv (1F6) | CD8α hinge | CD28 | CD28 | DAP12 | — |
| CAT-CD70-158 | CD8α SP | CD70 scFv (1F6) | CD8α hinge | CD8a | 4-1BB | DAP12 | — |
| CAT-CD70-159 | CD8α SP | CD70 scFv (1F6) | CD8α hinge | CD8a | OX40 | DAP12 | — |
| CAT-CD70-160 | CD8α SP | CD70 scFv (1F6) | CD8α hinge | CD8a | DAP10 | DAP12 | — |
| CAT-CD70-161 | CD8α SP | CD70 scFv (1F6) | CD8α hinge | CD28 | CD28 | FCER1G | — |
| CAT-CD70-162 | CD8α SP | CD70 scFv (1F6) | CD8α hinge | CD8a | 4-1BB | FCER1G | — |
| CAT-CD70-163 | CD8α SP | CD70 scFv (1F6) | CD8α hinge | CD8a | OX40 | FCER1G | — |
| CAT-CD70-164 | CD8α SP | CD70 scFv (1F6) | CD8α hinge | CD8a | DAP10 | FCER1G | — |
| CAT-CD70-278 | CD8α SP | CD70 scFv (1F6) | CD8a short | CD8a | 4-1BB | CD3z | — |
| CAT-CD70-127 | CD8α SP | CD70 scFv (1F6) | CD8α hinge | CD8a | 4-1BB | CD3z | — |
| CAT-CD70-291 | CD8α SP | CD70 scFv (1F6) | IgG1 short hinge | CD8a | 4-1BB | CD3z | — |
| CAT-CD70-281 | CD8α SP | CD70 scFv (1F6) | IgG4 short hinge | CD8a | 4-1BB | CD3z | — |
| CAT-CD70-280 | CD8α SP | CD70 scFv (1F6) | IgG4 hinge-CH3 | CD8a | 4-1BB | CD3z | — |
| CAT-CD70-279 | CD8α SP | CD70 scFv (1F6) | IgG4 hinge- | CD8a | 4-1BB | CD3z | — |
| | | | CH2—CH3 | | | | |
| CAT-CD70-293 | CD8α SP | CD70 scFv (1F6) | IgG4 mutant | CD8a | 4-1BB | CD3z | — |
| CAT-CD70-294 | CD8α SP | CD70 scFv (1F6) | CD8a short | CD8a | DAP10 | CD3z | — |
| CAT-CD70-148 | CD8α SP | CD70 scFv (1F6) | CD8α hinge | CD8a | DAP10 | CD3z | — |
| CAT-CD70-295 | CD8α SP | CD70 scFv (1F6) | IgG1 short hinge | CD8a | DAP10 | CD3z | — |
| CAT-CD70-296 | CD8α SP | CD70 scFv (1F6) | IgG4 short | CD8a | DAP10 | CD3z | — |
| CAT-CD70-297 | CD8α SP | CD70 scFv (1F6) | IgG4 hinge-CH3 | CD8a | DAP10 | CD3z | — |
| CAT-CD70-298 | CD8α SP | CD70 scFv (1F6) | IgG4 hinge- | CD8a | DAP10 | CD3z | — |
| | | | CH2—CH3 | | | | |
| CAT-CD70-299 | CD8α SP | CD70 scFv (1F6) | IgG4 mutant | CD8a | DAP10 | CD3z | — |
| CAT-CD70-300 | CD8α SP | CD70 scFv (1F6) | CD8a short | CD8a | OX40 | CD3z | — |
| CAT-CD70-150 | CD8α SP | CD70 scFv (1F6) | CD8α hinge | CD8a | OX40 | CD3z | — |
| CAT-CD70-301 | CD8α SP | CD70 scFv (1F6) | IgG1 short hinge | CD8a | OX40 | CD3z | — |
| CAT-CD70-302 | CD8α SP | CD70 scFv (1F6) | IgG4 short | CD8a | OX40 | CD3z | — |
| CAT-CD70-303 | CD8α SP | CD70 scFv (1F6) | IgG4 hinge-CH3 | CD8a | OX40 | CD3z | — |
| CAT-CD70-304 | CD8α SP | CD70 scFv (1F6) | IgG4 hinge- | CD8a | OX40 | CD3z | — |
| | | | CH2—CH3 | | | | |
| CAT-CD70-305 | CD8α SP | CD70 scFv (1F6) | IgG4 mutant | CD8a | OX40 | CD3z | — |
| CAT-CD70-306 | CD8α SP | CD70 scFv (1F6) | CD8a short | CD28 | CD28 | DAP12 | — |
| CAT-CD70-157 | CD8α SP | CD70 scFv (1F6) | CD8α hinge | CD28 | CD28 | DAP12 | — |
| CAT-CD70-307 | CD8α SP | CD70 scFv (1F6) | IgG1 short hinge | CD28 | CD28 | DAP12 | — |
| CAT-CD70-308 | CD8α SP | CD70 scFv (1F6) | IgG4 short | CD28 | CD28 | DAP12 | — |
| CAT-CD70-309 | CD8α SP | CD70 scFv (1F6) | IgG4 hinge-CH3 | CD28 | CD28 | DAP12 | — |
| CAT-CD70-310 | CD8α SP | CD70 scFv (1F6) | IgG4 hinge- | CD28 | CD28 | DAP12 | — |
| | | | CH2—CH3 | | | | |
| CAT-CD70-311 | CD8α SP | CD70 scFv (1F6) | IgG4 mutant | CD28 | CD28 | DAP12 | — |
| CAT-CD70-312 | CD8α SP | CD70 scFv (1F6) | CD8a short | CD28 | CD28 | CD3z | — |
| CAT-CD70-134 | CD8α SP | CD70 scFv (1F6) | CD8α hinge | CD28 | CD28 | CD3z | — |
| CAT-CD70-360 | CD8α SP | CD70 scFv (1F6) | IgG1 short hinge | CD28 | CD28 | CD3z | — |
| CAT-CD70-313 | CD8α SP | CD70 scFv (1F6) | IgG4 short | CD28 | CD28 | CD3z | — |
| CAT-CD70-314 | CD8α SP | CD70 scFv (1F6) | IgG4 hinge-CH3 | CD28 | CD28 | CD3z | — |
| CAT-CD70-315 | CD8α SP | CD70 scFv (1F6) | IgG4 hinge- | CD28 | CD28 | CD3z | — |
| | | | CH2—CH3 | | | | |
| CAT-CD70-316 | CD8α SP | CD70 scFv (1F6) | IgG4 mutant | CD28 | CD28 | CD3z | — |
| CAT-CD70-317 | CD8α SP | CD70 scFv (1F6) | CD8a short | CD28 | CD28 | OX40L | CD3z |
| CAT-CD70-318 | CD8α SP | CD70 scFv (1F6) | CD8α hinge | CD28 | CD28 | OX40L | CD3z |
| CAT-CD70-319 | CD8α SP | CD70 scFv (1F6) | IgG1 short hinge | CD28 | CD28 | OX40L | CD3z |
| CAT-CD70-320 | CD8α SP | CD70 scFv (1F6) | IgG4 short | CD28 | CD28 | OX40L | CD3z |
| CAT-CD70-321 | CD8α SP | CD70 scFv (1F6) | IgG4 hinge-CH3 | CD28 | CD28 | OX40L | CD3z |
| CAT-CD70-322 | CD8α SP | CD70 scFv (1F6) | IgG4 hinge- | CD28 | CD28 | OX40L | CD3z |
| | | | CH2—CH3 | | | | |
| CAT-CD70-323 | CD8α SP | CD70 scFv (1F6) | IgG4 mutant | CD28 | CD28 | OX40L | CD3z |
| CAT-CD70-324 | CD8α SP | CD70 scFv (1F6) | CD8a short | CD8a | 2B4 | CD3z | — |
| CAT-CD70-147 | CD8α SP | CD70 scFv (1F6) | CD8α hinge | CD8a | 2B4 | CD3z | — |
| CAT-CD70-325 | CD8α SP | CD70 scFv (1F6) | IgG1 short hinge | CD8a | 2B4 | CD3z | — |
| CAT-CD70-326 | CD8α SP | CD70 scFv (1F6) | IgG4 short | CD8a | 2B4 | CD3z | — |
| CAT-CD70-327 | CD8α SP | CD70 scFv (1F6) | IgG4 hinge-CH3 | CD8a | 2B4 | CD3z | — |
| CAT-CD70-328 | CD8α SP | CD70 scFv (1F6) | IgG4 hinge- | CD8a | 2B4 | CD3z | — |
| | | | CH2—CH3 | | | | |
| CAT-CD70-329 | CD8α SP | CD70 scFv (1F6) | IgG4 mutant | CD8a | 2B4 | CD3z | — |
| CAT-CD70-330 | CD8α SP | CD70 scFv (1F6) | CD8α hinge | CD8a | DAP12 | CD3z | — |
| CAT-CD70-149 | CD8α SP | CD70 scFv (1F6) | CD8α hinge | CD8a | DAP12 | CD3z | — |
| CAT-CD70-331 | CD8α SP | CD70 scFv (1F6) | IgG1 short hinge | CD8a | DAP12 | CD3z | — |
| CAT-CD70-332 | CD8α SP | CD70 scFv (1F6) | IgG4 short | CD8a | DAP12 | CD3z | — |
| CAT-CD70-333 | CD8α SP | CD70 scFv (1F6) | IgG4 hinge-CH3 | CD8a | DAP12 | CD3z | — |
| CAT-CD70-334 | CD8α SP | CD70 scFv (1F6) | IgG4 hinge- | CD8a | DAP12 | CD3z | — |
| | | | CH2—CH3 | | | | |
| CAT-CD70-335 | CD8α SP | CD70 scFv (1F6) | IgG4 mutant | CD8a | DAP12 | CD3z | — |
| CAT-CD70-336 | CD8α SP | CD70 scFv (1F6) | CD8a short | CD8a | | CD3z | — |
| CAT-CD70-145 | CD8α SP | CD70 scFv (1F6) | CD8α hinge | CD8a | | CD3z | — |
| CAT-CD70-337 | CD8α SP | CD70 scFv (1F6) | IgG1 short hinge | CD8a | | CD3z | — |
| CAT-CD70-338 | CD8α SP | CD70 scFv (1F6) | IgG4 short | CD8a | | CD3z | — |
| CAT-CD70-339 | CD8α SP | CD70 scFv (1F6) | IgG4 hinge-CH3 | CD8a | | CD3z | — |
| CAT-CD70-340 | CD8α SP | CD70 scFv (1F6) | IgG4 hinge- | CD8a | | CD3z | — |
| | | | CH2—CH3 | | | | |
| CAT-CD70-341 | CD8α SP | CD70 scFv (1F6) | IgG4 mutant | CD8a | | CD3z | — |
| CAT-CD70-342 | CD8α SP | CD70 scFv (1F6) | CD8a short | CD8a | 4-1BB | DAP12 | — |
| CAT-CD70-158 | CD8α SP | CD70 scFv (1F6) | CD8α hinge | CD8a | 4-1BB | DAP12 | — |
| CAT-CD70-343 | CD8α SP | CD70 scFv (1F6) | IgG1 short hinge | CD8a | 4-1BB | DAP12 | — |
| CAT-CD70-344 | CD8α SP | CD70 scFv (1F6) | IgG4 short | CD8a | 4-1BB | DAP12 | — |
| CAT-CD70-345 | CD8α SP | CD70 scFv (1F6) | IgG4 hinge-CH3 | CD8a | 4-1BB | DAP12 | — |
| CAT-CD70-346 | CD8α SP | CD70 scFv (1F6) | IgG4 hinge- | CD8a | 4-1BB | DAP12 | — |
| | | | CH2—CH3 | | | | |
| CAT-CD70-347 | CD8α SP | CD70 scFv (1F6) | IgG4 mutant | CD8a | 4-1BB | DAP12 | — |
| CAT-CD70-348 | CD8α SP | CD70 scFv (1F6) | CD8a short | CD8a | OX40 | DAP12 | — |
| CAT-CD70-159 | CD8α SP | CD70 scFv (1F6) | CD8α hinge | CD8a | OX40 | DAP12 | — |
| CAT-CD70-349 | CD8α SP | CD70 scFv (1F6) | IgG1 short hinge | CD8a | OX40 | DAP12 | — |
| CAT-CD70-350 | CD8α SP | CD70 scFv (1F6) | IgG4 short | CD8a | OX40 | DAP12 | — |
| CAT-CD70-351 | CD8α SP | CD70 scFv (1F6) | IgG4 hinge-CH3 | CD8a | OX40 | DAP12 | — |
| CAT-CD70-352 | CD8α SP | CD70 scFv (1F6) | IgG4 hinge- | CD8a | OX40 | DAP12 | — |
| | | | CH2—CH3 | | | | |
| CAT-CD70-353 | CD8α SP | CD70 scFv (1F6) | IgG4 mutant | CD8a | OX40 | DAP12 | — |
| CAT-CD70-354 | CD8α SP | CD70 scFv (1F6) | CD8a short | CD28 | CD28 | FCER1G | — |
| CAT-CD70-161 | CD8α SP | CD70 scFv (1F6) | CD8α hinge | CD28 | CD28 | FCER1G | — |
| CAT-CD70-355 | CD8α SP | CD70 scFv (1F6) | IgG1 short hinge | CD28 | CD28 | FCER1G | — |
| CAT-CD70-356 | CD8α SP | CD70 scFv (1F6) | IgG4 short | CD28 | CD28 | FCER1G | — |
| CAT-CD70-357 | CD8α SP | CD70 scFv (1F6) | IgG4 hinge-CH3 | CD28 | CD28 | FCER1G | — |
| CAT-CD70-358 | CD8α SP | CD70 scFv (1F6) | IgG4 hinge- | CD28 | CD28 | FCER1G | — |
| | | | CH2—CH3 | | | | |
| CAT-CD70-359 | CD8α SP | CD70 scFv (1F6) | IgG4 mutant | CD28 | CD28 | FCER1G | — |

Table 6 provides exemplary sequences of the anti-CD70 CAR constructs disclosed herein. In some embodiments, an immune cell (e.g., NK cell) or population of immune cells (e.g., NK cells) described herein is genetically modified to express at least one of the exemplary anti-CD70 CAR constructs provided in Table 6. In some embodiments, the CAR of any one of SEQ ID NOs: 637, 639, 641, 643, 645, 647, 700, 2561-2593 does not comprise the indicated signal peptide. In some embodiments, an immune cell (e.g., NK cell) or population of immune cells (e.g., NK cells) described herein comprises a chimeric antigen receptor comprising an amino acid sequence that is at least 90%, at least 91%, at least 92%, at least 93%, at least 94%, at least 95%, at least 96%, at least 97%, at least 98%, at least 99% or at least 100% identical to the amino acid sequence of any one of SEQ ID NOs: 637, 639, 641, 643, 645, 647, 700, 2561-2593, 2697-2736 or 2737-2882.

TABLE 6
Exemplary sequences of anti-CD70 CAR constructs

| Exemplary CAR | | SEQ |
|---|---|---|
| Name and Domains | Amino Acid Sequence | ID NO: |
| CAT-70-001 | MALPVTALLLPLALLLHAARPQVQLVQSGAEVKKPGASVKVSCKASGYTFT | 637 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDFWVLVVVGGVLACYSLLVTVAFIIFWV [symbol] | |
| signaling domain | [symbol] [symbol] RVKFSRSADAPAYQQGQNQ | |
| | LYNELNLGRREEYDVLDKRRGRDPEMGGKPRRKNPQEGLYNELQKDKMAEA | |
| | YSEIGMKGERRRGKGHDGLYQGLSTATKDTYDALHMQALPPR | |
| CAT-70-002 | MALPVTALLLPLALLLHAARPQVQLVQSGAEVKKPGASVKVSCKASGYTFT | 639 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, NKG2D | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDPFFFCCFIAVAMGIRFIIMVAIWSAVFLNS [symbol] | |
| signaling domain | [symbol] RVKFSRSADAPAYQQGQNQLYNELNLGRREEYD | |
| | VLDKRRGRDPEMGGKPRRKNPQEGLYNELQKDKMAEAYSEIGMKGERRRGK | |
| | GHDGLYQGLSTATKDTYDALHMQALPPR | |
| CAT-70-003 | MALPVTALLLPLALLLHAARPQVQLVQSGAEVKKPGASVKVSCKASGYTFT | 641 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, NKG2D | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDPFFFCCFIAVAMGIRFIIMVAIWSAVFLNS [symbol] | |
| signaling domain | [symbol] [symbol] RVKFS | |
| | RSADAPAYQQGQNQLYNELNLGRREEYDVLDKRRGRDPEMGGKPRRKNPQE | |
| | GLYNELQKDKMAEAYSEIGMKGERRRGKGHDGLYQGLSTATKDTYDALHMQ | |
| | ALPPR | |
| CAT-70-004 | MARPHPWWLCVLGTLVGLSATPAPKSCPERHYWAQGKLCCQMCEPGTFLVK | 643 |
| (Construct #1) | DCDQHRKAAQCDPCIPGVSFSPDHHTRPHCESCRHCNSGLLVRNCTITANA | |
| CD27 signal peptide, | ECACRNGWQCRDKECTECDPLPNPSLTARSSQALSPHPQPTHLPYVSEMLE | |
| CD27 extracellular | ARTAGHMQTLADFRQLPARTLSTHWPPQRSLCSSDFIRILVIFSGMFLVFT | |
| domain, CD27 | LAGALFL [symbol] [symbol] | |
| transmembrane domain, | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTAT | |
| [symbol] CD3z | KDTYDALHMQALPPR | |
| signaling domain | | |
| CAT-70-005 | MARPHPWWLCVLGTLVGLSATPAPKSCPERHYWAQGKLCCQMCEPGTFLVK | 645 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, CD28 | ARTAGHMQTLADFRQLPARTLSTHWPPQRSLCSSDFIRFWVLVVVGGVLAC | |
| transmembrane domain, | YSLLVTVAFIIFWV [symbol] [symbol] | |
| [symbol] | [symbol] RVKFSRSADAPAYQQGQNQLYNELNLGRREEYDVLDKRRGRDPEMGG | |
| [symbol] CD3z | KPRRKNPQEGLYNELQKDKMAEAYSEIGMKGERRRGKGHDGLYQGLSTATK | |
| signaling domain | DTYDALHMQALPPR | |
| CAT-70-006 | MARPHPWWLCVLGTLVGLSATPAPKSCPERHYWAQGKLCCQMCEPGTFLVK | 647 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, NKG2D | ARTAGHMQTLADFRQLPARTLSTHWPPQRSLCSSDFIRPFFFCCFIAVAMG | |
| transmembrane domain, | IRFIIMVAIWSAVFLNS [symbol] RVKFSRSADA | |
| [symbol] | PAYQQGQNQLYNELNLGRREEYDVLDKRRGRDPEMGGKPRRKNPQEGLYNE | |
| [symbol] CD3z | LQKDKMAEAYSEIGMKGERRRGKGHDGLYQGLSTATKDTYDALHMQALPPR | |
| signaling domain | | |
| CAT-70-007 | MARPHPWWLCVLGTLVGLSATPAPKSCPERHYWAQGKLCCQMCEPGTFLVK | 700 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, NKG2D | ARTAGHMQTLADFRQLPARTLSTHWPPQRSLCSSDFIRPFFFCCFIAVAMG | |
| transmembrane domain, | IRFIIMVAIWSAVFLNS [symbol] [symbol] | |
| [symbol] | [symbol] RVKFSRSADAPAYQQGQNQLYNELNLGRREEYD | |
| [symbol] CD3z | VLDKRRGRDPEMGGKPRRKNPQEGLYNELQKDKMAEAYSEIGMKGERRRGK | |
| signaling domain | GHDGLYQGLSTATKDTYDALHMQALPPR | |
| CAT-CD70-119 | MARPHPWWLCVLGTLVGLSATPAPKSCPERHYWAQGKLCCQMCEPGTFLVK | 2561 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, CD27 | ARTAGHMQTLADFRQLPARTLSTHWPPQRSLCSSDFIRILVIFSGMFLVFT | |
| transmembrane domain, | LAGALFL [symbol] [symbol] R | |
| [symbol] | VKFSRSADAPAYQQGQNQLYNELNLGRREEYDVLDKRRGRDPEMGGKPRRK | |
| [symbol] CD3z | NPQEGLYNELQKDKMAEAYSEIGMKGERRRGKGHDGLYQGLSTATKDTYDA | |
| signaling domain | LHMQALPPR | |
| CAT-CD70-122 | MARPHPWWLCVLGTLVGLSATPAPKSCPERHYWAQGKLCCQMCEPGTFLVK | 2562 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, CD8α hinge, | ARTAGHMQTLADFRQLPARTLSTHWPPQRSLCSSDFIRFVPVFLPAKPTTT | |
| CD8α transmembrane | PAPRPPTPAPTIASQPLSLRPEACRPAAGGAVHTRGLDFACDIYIWAPLAG | |
| domain, [symbol] | TCGVLLLSLVITLYCNHRN [symbol] [symbol] | |
| [symbol] | [symbol] RVKFSRSADAPAYQQGQNQLYNELNLGRREEYDVLDKRRG | |
| CD3z signaling domain | RDPEMGGKPRRKNPQEGLYNELQKDKMAEAYSEIGMKGERRRGKGHDGLYQ | |
| | GLSTATKDTYDALHMQALPPR | |
| CAT-CD70-124 | MARPHPWWLCVLGTLVGLSATPAPKSCPERHYWAQGKLCCQMCEPGTFLVK | 2563 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, CD27 | ARTAGHMQTLADFRQLPARTLSTHWPPQRSLCSSDFIRILVIFSGMFLVFT | |
| transmembrane domain, | LAGALFL [symbol] [symbol] RVK | |
| [symbol] | FSRSADAPAYQQGQNQLYNELNLGRREEYDVLDKRRGRDPEMGGKPRRKNP | |
| [symbol] CD3z | QEGLYNELQKDKMAEAYSEIGMKGERRRGKGHDGLYQGLSTATKDTYDALH | |
| signaling domain | MQALPPR | |
| CAT-CD70-125 | MARPHPWWLCVLGTLVGLSATPAPKSCPERHYWAQGKLCCQMCEPGTFLVK | 2564 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, CD8α hinge, | ARTAGHMQTLADFRQLPARTLSTHWPPQRSLCSSDFIRFVPVFLPAKPTTT | |
| CD8α tmnsmembrane | PAPRPPTPAPTIASQPLSLRPEACRPAAGGAVHTRGLDFACDIYIWAPLAG | |
| domain, [symbol] | TCGVLLLSLVITLYCNHRN [symbol] [symbol] | |
| [symbol] | [symbol] RVKFSRSADAPAYQQGQNQLYNELNLGRREEYDVLDKRRGRD | |
| CD3z signaling domain | PEMGGKPRRKNPQEGLYNELQKDKMAEAYSEIGMKGERRRGKGHDGLYQGL | |
| | STATKDTYDALHMQALPPR | |
| CAT-CD70-127 | MALPVTALLLPLALLLHAARPQVQLVQSGAEVKKPGASVKVSCKASGYTFT | 2565 |
| (Construct #2) | NYGMNWVRQAPGQGLKWMGWINTYTGEPTYADAFKGRVTMTRDTSISTAYM | |
| CD8α signal peptide, | ELSRLRSDDTAVYYCARDYGDYGMDYWGQGTTVTVSSGGGGSGGGGSGGGG | |
| CD70 scFv (1F6), | SGDIVMTQSPDSLAVSLGERATINCRASKSVSTSGYSFMHWYQQKPGQPPK | |
| CD8α hinge, CD8α | LLIYLASNLESGVPDRFSGSGSGTDFTLTISSLQAEDVAVYYCQHSREVPW | |
| transmembrane domain, | TFGQGTKVEIKFVPVFLPAKPTTTPAPRPPTPAPTIASQPLSLRPEACRPA | |
| [symbol] | AGGAVHTRGLDFACDIYIWAPLAGTCGVLLLSLVITLYCNHRN [symbol] | |
| [symbol] CD3z | [symbol] [symbol] RVKFSRSADAPAYQQG | |
| signaling domain | QNQLYNELNLGRREEYDVLDKRRGRDPEMGGKPRRKNPQEGLYNELQKDKM | |
| | AEAYSEIGMKGERRRGKGHDGLYQGLSTATKDTYDALHMQALPPR | |
| CAT-CD70-130 | MALPVTALLLPLALLLHAARPQVQLVQSGAEVKKPGASVKVSCKASGYTFT | 2566 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKEPKSCDKTHTCPPCPAPELLGGPSVFLFPPKPKDTLMISR | |
| [symbol] CD3z | TPEVTCVVVDVSHEDPEVKFNWYVDGVEVHNAKTKPREEQYNSTYRVVSVL | |
| signaling domain | TVLHQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFFLYS | |
| | KLTVDKSRWQQGNVFSCSVMHEALHNHYTQKSLSLSPGKFWVLVVVGGVLA | |
| | CYSLLVTVAFIIFWV [symbol] [symbol] | |
| | [symbol] RVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTAT | |
| | KDTYDALHMQALPPR | |
| CAT-CD70-133 | MALPVTALLLPLALLLHAARPQVQLVQSGAEVKKPGASVKVSCKASGYTFT | 2567 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD28 hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKIEVMYPPPYLDNEKSNGTIIHVKGKHLCPSPLFPGPSKPF | |
| [symbol] CD3z | WVLVVVGGVLACYSLLVTVAFIIFWV [symbol] [symbol] | |
| signaling domain | [symbol] RVKFSRSADAPAYQQGQNQLYNELNLGRREEYDVL | |
| | DKRRGRDPEMGGKPRRKNPQEGLYNELQKDKMAEAYSEIGMKGERRRGKGH | |
| | DGLYQGLSTATKDTYDALHMQALPPR | |
| CAT-CD70-135 | MALPVTALLLPLALLLHAARPQVQLVQSGAEVKKPGASVKVSCKASGYTFT | 2568 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDIYIWAPLAGTCGVLLLSLVITLYCNHRN [symbol] | |
| signaling domain | [symbol] [symbol] RVKFSRSADAPAYQQGQN | |
| | QLYNELNLGRREEYDVLDKRRGRDPEMGGKPRRKNPQEGLYNELQKDKMAE | |
| | AYSEIGMKGERRRGKGHDGLYQGLSTATKDTYDALHMQALPPR | |
| CAT-CD70-136 | MARPHPWWLCVLGTLVGLSATPAPKSCPERHYWAQGKLCCQMCEPGTFLVK | 2569 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, CD27 | ARTAGHMQTLADFRQLPARTLSTHWPPQRSLCSSDFIRILVIFSGMFLVFT | |
| transmembrane domain, | LAGALFL [symbol] [symbol] | |
| [symbol] | [symbol] YFLGRLVPRGRGAAEAATRKQRITETESPYQELQGQRSDVYSDLNT | |
| [symbol] DAP12 | QRPYYK | |
| signaling domain | | |
| CAT-CD70-137 | MARPHPWWLCVLGTLVGLSATPAPKSCPERHYWAQGKLCCQMCEPGTFLVK | 2570 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, CD27 | ARTAGHMQTLADFRQLPARTLSTHWPPQRSLCSSDFIRILVIFSGMFLVFT | |
| transmembrane domain, | LAGALFL [symbol] [symbol] | |
| [symbol] | [symbol] RLKIQVRKAAITSYEKSDGVYTGLSTRNQETYETLKHEKPPQ | |
| [symbol] FCER1G | | |
| signaling domain | | |
| CAT-CD70-140 | MARPHPWWLCVLGTLVGLSATPAPKSCPERHYWAQGKLCCQMCEPGTFLVK | 2571 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, DAP10 | ARTAGHMQTLADFRQLPARTLSTHWPPQRSLCSSDFIRLLAGLVAADAVAS | |
| transmembrane domain, | LLIVGAVF [symbol] [symbol] RVKFSRSADAPAYQQGQNQ | |
| [symbol] | LYNELNLGRREEYDVLDKRRGRDPEMGGKPRRKNPQEGLYNELQKDKMAEA | |
| [symbol] CD3z | YSEIGMKGERRRGKGHDGLYQGLSTATKDTYDALHMQALPPR | |
| signaling domain | | |
| CAT-CD70-141 | MARPHPWWLCVLGTLVGLSATPAPKSCPERHYWAQGKLCCQMCEPGTFLVK | 2572 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, DAP12 | ARTAGHMQTLADFRQLPARTLSTHWPPQRSLCSSDFIRGVLAGIVMGDLVL | |
| transmembrane domain, | TVLIALAV [symbol] [symbol] | |
| [symbol] | [symbol] RVKFSRSADAPAYQQGQNQLYNELNLGRREEYDVLDKRRGRD | |
| [symbol] CD3z | PEMGGKPRRKNPQEGLYNELQKDKMAEAYSEIGMKGERRRGKGHDGLYQGL | |
| signaling domain | STATKDTYDALHMQALPPR | |
| CAT-CD70-142 | MARPHPWWLCVLGTLVGLSATPAPKSCPERHYWAQGKLCCQMCEPGTFLVK | 2573 |
| CD27 signal peptide, | DCDQHRKAAQCDPCIPGVSFSPDHHTRPHCESCRHCNSGLLVRNCTITANA | |
| CD27 extracellular | ECACRNGWQCRDKECTECDPLPNPSLTARSSQALSPHPQPTHLPYVSEMLE | |
| domain, DAP12 | ARTAGHMQTLADFRQLPARTLSTHWPPQRSLCSSDFIRGVLAGIVMGDLVL | |
| transmembrane domain, | TVLIALAV [symbol] [symbol] | |
| [symbol] | [symbol] | |
| [symbol] | | |
| CAT-CD70-143 | MALPVTALLLPLALLLHAARPQVQLVQSGAEVKKPGASVKVSCKASGYTFT | 2574 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKEPKSCDKTHTCPPCPAPELLGGPSVFLFPPKPKDTLMISR | |
| [symbol] | TPEVTCVVVDVSHEDPEVKFNWYVDGVEVHNAKTKPREEQYNSTYRVVSVL | |
| | TVLHQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFFLYS | |
| | KLTVDKSRWQQGNVFSCSVMHEALHNHYTQKSLSLSPGKFWVLVVVGGVLA | |
| | CYSLLVTVAFIIFWV [symbol] [symbol] | |
| | AAYRS [symbol] | |
| CAT-CD70-144 | MALPVTALLLPLALLLHAARPQVQLVQSGAEVKKPGASVKVSCKASGYTFT | 2575 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] | AGGAVHTRGLDFACDIYIWAPLAGTCGVLLLSLVITLYCNHRNRKRGRECKL | |
| | LYIFKQPFMRPVQTTQEEDGCSCRFPEEEEGGCEL | |
| CAT-CD70-145 | MALPVTALLLPLALLLHAARPQVQLVQSGAEVKKPGASVKVSCKASGYTFT | 2576 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| CD3z signaling domain | TFGQGTKVEIKFVPVFLPAKPTTTPAPRPPTPAPTIASQPLSLRPEACRPA | |
| | AGGAVHTRGLDFACDIYIWAPLAGTCGVLLLSLVITLYCNHRNRVKFSRSA | |
| | DAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALP | |
| | PR | |
| CAT-CD70-146 | MALPVTALLLPLALLLHAARPQVQLVQSGAEVKKPGASVKVSCKASGYTFT | 2577 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] , 4-1BB | AGGAVHTRGLDFACDIYIWAPLAGTCGVLLLSLVITLYCNHRN [symbol] | |
| signaling domain | [symbol] [symbol] RKRGRKKLLYIFKQPF | |
| | MRPVQTTQEEDGCSCRFPEEEEGGCEL | |
| CAT-CD70-147 | MALPVTALLLPLALLLHAARPQVQLVQSGAEVKKPGASVKVSCKASGYTFT | 2578 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| CD3z signaling domain | AGGAVHTRGLDFACDIYIWAPLAGTCGVLLLSLVITLYCNHRN [symbol] | |
| | [symbol] [symbol] [symbol] | |
| | [symbol] [symbol] [symbol] | |
| | [symbol] RVKFSRSADAPAYQQGQNQLYNELNLGRREEYDVLDKRRGR | |
| | DPEMGGKPRRKNPQEGLYNELQKDKMAEAYSEIGMKGERRRGKGHDGLYQG | |
| | LSTATKDTYDALHMQALPPR | |
| CAT-CD70-148 | MALPVTALLLPLALLLHAARPQVQLVQSGAEVKKPGASVKVSCKASGYTFT | 2579 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDIYIWAPLAGTCGVLLLSLVITLYCNHRN [symbol] | |
| signaling domain | [symbol] RVKFSRSADAPAYQQGQNQLYNELNLGRREEYDVL | |
| | DKRRGRDPEMGGKPRRKNPQEGLYNELQKDKMAEAYSEIGMKGERRRGKGH | |
| | DGLYQGLSTATKDTYDALHMQALPPR | |
| CAT-CD70-149 | MALPVTALLLPLALLLHAARPQVQLVQSGAEVKKPGASVKVSCKASGYTFT | 2580 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDIYIWAPLAGTCGVLLLSLVITLYCNHRN [symbol] | |
| signaling domain | [symbol] [symbol] [symbol] RVKFSRS | |
| | ADAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQAL | |
| | PPR | |
| CAT-CD70-150 | MALPVTALLLPLALLLHAARPQVQLVQSGAEVKKPGASVKVSCKASGYTFT | 2581 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDIYIWAPLAGTCGVLLLSLVITLYCNHRN [symbol] | |
| signaling domain | [symbol] [symbol] RVKFSRSADAPAYQQGQ | |
| | NQLYNELNLGRREEYDVLDKRRGRDPEMGGKPRRKNPQEGLYNELQKDKMA | |
| | EAYSEIGMKGERRRGKGHDGLYQGLSTATKDTYDALHMQALPPR | |
| CAT-CD70-153 | MALPVTALLLPLALLLHAARPQVQLVQSGAEVKKPGASVKVSCKASGYTFT | 2582 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, NKG2D | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| CD3z signaling domain | AGGAVHTRGLDFACDPFFFCCFIAVAMGIRFIIMVAIWSAVFLNS [symbol] | |
| | [symbol] [symbol] [symbol] | |
| | [symbol] [symbol] [symbol] | |
| | [symbol] RVKFSRSADAPAYQQGQNQLYNELNLGRREEYDVLDKRR | |
| | GRDPEMGGKPRRKNPQEGLYNELQKDKMAEAYSEIGMKGERRRGKGHDGLY | |
| | QGLSTATKDTYDALHMQALPPR | |
| CAT-CD70-154 | MALPVTALLLPLALLLHAARPQVQLVQSGAEVKKPGASVKVSCKASGYTFT | 2583 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, DAP10 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDLLAGLVAADAVASLLIVGAVF [symbol] | |
| signaling domain | [symbol] RVKFSRSADAPAYQQGQNQLYNELNLGRREEYDVLDKRRGRD | |
| | PEMGGKPRRKNPQEGLYNELQKDKMAEAYSEIGMKGERRRGKGHDGLYQGL | |
| | STATKDTYDALHMQALPPR | |
| CAT-CD70-155 | MALPVTALLLPLALLLHAARPQVQLVQSGAEVKKPGASVKVSCKASGYTFT | 2584 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, DAP12 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] CD3z | AGGAVHTRGLDFACDGVLAGIVMGDLVLTVLIALAV [symbol] | |
| signaling domain | [symbol] [symbol] RVKFSRSADAPAYQ | |
| | QGQNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| CAT-CD70-156 | MALPVTALLLPLALLLHAARPQVQLVQSGAEVKKPGASVKVSCKASGYTFT | 2585 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, DAP12 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] | AGGAVHTRGLDFACDGVLAGIVMGDLVLTVLIALAV [symbol] | |
| | [symbol] [symbol] | |
| CAT-CD70-157 | MALPVTALLLPLALLLHAARPQVQLVQSGAEVKKPGASVKVSCKASGYTFT | 2586 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] DAP12 | AGGAVHTRGLDFACDFWVLVVVGGVLACYSLLVTVAFIIFWV [symbol] | |
| signaling domain | [symbol] [symbol] YFLGRLVPRGRGAAEAATR | |
| | KQRITETESPYQELQGQRSDVYSDLNTQRPYYK | |
| CAT-CD70-158 | MALPVTALLLPLALLLHAARPQVQLVQSGAEVKKPGASVKVSCKASGYTFT | 2587 |
| CD8αsignal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] DAP12 | AGGAVHTRGLDFACDIYIWAPLAGTCGVLLLSLVITLYCNHRN [symbol] | |
| signaling domain | [symbol] [symbol] YFLGRLVPRGRGAAEA | |
| | ATRKQRITETESPYQELQGQRSDVYSDLNTQRPYYK | |
| CAT-CD70-159 | MALPVTALLLPLALLLHAARPQVQLVQSGAEVKKPGASVKVSCKASGYTFT | 2588 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] DAP12 | AGGAVHTRGLDFACDIYIWAPLAGTCGVLLLSLVITLYCNHRN [symbol] | |
| signaling domain | [symbol] [symbol] YFLGRLVPRGRGAAEAA | |
| | TRKQRITETESPYQELQGQRSDVYSDLNTQRPYYK | |
| CAT-CD70-160 | MALPVTALLLPLALLLHAARPQVQLVQSGAEVKKPGASVKVSCKASGYTFT | 2589 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] DAP12 | AGGAVHTRGLDFACDIYIWAPLAGTCGVLLLSLVITLYCNHRN [symbol] | |
| signaling domain | [symbol] YFLGRLVPRGRGAAEAATRKQRITETESPYQELQG | |
| | QRSDVYSDLNTQRPYYK | |
| CAT-CD70-161 | MALPVTALLLPLALLLHAARPQVQLVQSGAEVKKPGASVKVSCKASGYTFT | 2590 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] FCER1G | AGGAVHTRGLDFACDFWVLVVVGGVLACYSLLVTVAFIIFWV [symbol] | |
| signaling domain | [symbol] [symbol] RLKIQVRKAAITSYEKSDG | |
| | VYTGLSTRNQETYETLKHEKPPQ | |
| CAT-CD70-162 | MALPVTALLLPLALLLHAARPQVQLVQSGAEVKKPGASVKVSCKASGYTFT | 2591 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] FCER1G | AGGAVHTRGLDFACDIYIWAPLAGTCGVLLLSLVITLYCNHRN [symbol] | |
| signaling domain | [symbol] [symbol] RLKIQVRKAAITSYEK | |
| | SDGVYTGLSTRNQETYETLKHEKPPQ | |
| CAT-CD70-163 | MALPVTALLLPLALLLHAARPQVQLVQSGAEVKKPGASVKVSCKASGYTFT | 2592 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] FCER1G | AGGAVHTRGLDFACDIYIWAPLAGTCGVLLLSLVITLYCNHRN [symbol] | |
| signaling domain | [symbol] [symbol] RLKIQVRKAAITSYEKS | |
| | DGVYTGLSTRNQETYETLKHEKPPQ | |
| CAT-CD70-164 | MALPVTALLLPLALLLHAARPQVQLVQSGAEVKKPGASVKVSCKASGYTFT | 2593 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKFVPVFLPAKPTTTPAPRPPTPAPTIASQPLSLRPEACRPA | |
| [symbol] , FCER1G | AGGAVHTRGLDFACDIYIWAPLAGTCGVLLLSLVITLYCNHRNLCARPRRS | |
| signaling domain | PAQEDGKVYINMPGRGRLKIQVRKAAITSYEKSDGVYTGLSTRNQETYETL | |
| | KHEKPPQ | |
| CAT-CD70-278 | MALPVTALLLPLALLLHAARPQVQLVQSGAEVKKPGASVKVSCKASGYTFT | 2737 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDIYIWAPLAGTCGVLLLSLVITLYCNHRN [symbol] | |
| CD3z signaling domain | [symbol] [symbol] RVKFSRSADAPAYQQGQNQLYNELNL | |
| | GRREEYDVLDKRRGRDPEMGGKPRRKNPQEGLYNELQKDKMAEAYSEIGMK | |
| | GERRRGKGHDGLYQGLSTATKDTYDALHMQALPPR | |
| CAT-CD 70-291 | MALPVTALLLPLALLLHAARPQVQLVQSGAEVKKPGASVKVSCKASGYTFT | 2739 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKAEPKSPDKTHTCPPCPKDPIYIWAPLAGTCGVLLLSLVIT | |
| [symbol] CD3z | LYCNHRN [symbol] [symbol] R | |
| signaling domain | VKFSRSADAPAYQQGQNQLYNELNLGRREEYDVLDKRRGRDPEMGGKPRRK | |
| | NPQEGLYNELQKDKMAEAYSEIGMKGERRRGKGHDGLYQGLSTATKDTYDA | |
| | LHMQALPPR | |
| CAT-CD 70-281 | MALPVTALLLPLALLLHAARPQVQLVQSGAEVKKPGASVKVSCKASGYTFT | 2741 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPIYIWAPLAGTCGVLLLSLVITLYCNHRN | |
| [symbol] CD3z | [symbol] [symbol] RVKFSRSA | |
| signaling domain | DAPAYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQALP | |
| | PR | |
| CAT-CD70-280 | MALPVTALLLPLALLLHAARPQVQLVQSGAEVKKPGASVKVSCKASGYTFT | 2743 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKESKYGPPCPSCPGQPREPQVYTLPPSQEEMTKNQVSLTCL | |
| [symbol] CD3z | VKGFYPSDIAVEWESNGQPENNYKTTPPVLDSDGSFFLYSRLTVDKSRWQE | |
| signaling domain | GNVFSCSVMHEALHNHYTQKSLSLSLGKIYIWAPLAGTCGVLLLSLVITLY | |
| | CNHRN [symbol] [symbol] RVK | |
| | FSRSADAPAYQQGQNQLYNELNLGRREEYDVLDKRRGRDPEMGGKPRRKNP | |
| | QEGLYNELQKDKMAEAYSEIGMKGERRRGKGHDGLYQGLSTATKDTYDALH | |
| | MQALPPR | |
| CAT-CD70-279 | MALPVTALLLPLALLLHAARPQVQLVQSGAEVKKPGASVKVSCKASGYTFT | 2745 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] RVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTAT | |
| | KDTYDALHMQALPPR | |
| CAT-CD70-293 | MALPVTALLLPLALLLHAARPQVQLVQSGAEVKKPGASVKVSCKASGYTFT | 2747 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSGFBRTSGYSFMHWYQQKPG | |
| CD8α transmembrane | QPPKLLIYLASNLESGVPDRFSGSGSGTDFTLTISSLQAEDVAVYYCQHSR | |
| domain, [symbol] | EVPWTFGQGTKVEIKESKYGPPCPSCPAPEFEGGPSVFLFPPKPKDTLMIS | |
| [symbol] | RTPEVTCVVVDVSQEDPEVQFNWYVDGVEVHNAKTKPREEQFQSTYRVVSV | |
| CD3z signaling domain | LTVLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFFLY | |
| | SRLTVDKSRWQEGNVFSCSVMHEALHNHYTQKSLSLSLGKIYIWAPLAGTC | |
| | GVLLLSLVITLYCNHRN [symbol] [symbol] | |
| | [symbol] RVKFSRSADAPAYQQGQNQLYNELNLGRREEYDVLDKRRGRD | |
| | PEMGGKPRRKNPQEGLYNELQKDKMAEAYSEIGMKGERRRGKGHDGLYQGL | |
| | STATKDTYDALHMQALPPR | |
| CAT-CD70-294 | MALPVTALLLPLALLLHAARPQVQLVQSGAEVKKPGASVKVSCKASGYTFT | 2749 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDIYIWAPLAGTCGVLLLSLVITLYCNHRN [symbol] | |
| CD3z signaling domain | [symbol] RVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQGLSTA | |
| | TKDTYDALHMQALPPR | |
| CAT-CD70-295 | MALPVTALLLPLALLLHAARPQVQLVQSGAEVKKPGASVKVSCKASGYTFT | 2751 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKAEPKSPDKTHTCPPCPKDPIYIWAPLAGTCGVLLLSLVIT | |
| [symbol] CD3z | LYCNHRN [symbol] RVKFSRSADAPAYQQGQNQL | |
| signaling domain | YNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| CAT-CD70-296 | MALPVTALLLPLALLLHAARPQVQLVQSGAEVKKPGASVKVSCKASGYTFT | 2753 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPIYIWAPLAGTCGVLLLSLVITLYCNHRN | |
| [symbol] CD3z | [symbol] RVKFSRSADAPAYQQGQNQLYNELNLG | |
| signaling domain | RREEYDVLDKRRGRDPEMGGKPRRKNPQEGLYNELQKDKMAEAYSEIGMKG | |
| | ERRRGKGHDGLYQGLSTATKDTYDALHMQALPPR | |
| CAT-CD70-297 | MALPVTALLLPLALLLHAARPQVQLVQSGAEVKKPGASVKVSCKASGYTFT | 2755 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPGQPREPQVYTLPPSQEEMTKNQVSLTCL | |
| [symbol] CD3z | VKGFYPSDIAVEWESNGQPENNYKTTPPVLDSDGSFFLYSRLTVDKSRWQE | |
| signaling domain | GNVFSCSVMHEALHNHYTQKSLSLSLGKIYIWAPLAGTCGVLLLSLVITLY | |
| | CNHRN [symbol] [symbol] RVKFSRSADAPAYQQGQNQLYN | |
| | ELNLGRREEYDVLDKRRGRDPEMGGKPRRKNPQEGLYNELQKDKMAEAYSE | |
| | IGMKGERRRGKGHDGLYQGLSTATKDTYDALHMQALPPR | |
| CAT-CD70-298 | MALPVTALLLPLALLLHAARPQVQLVQSGAEVKKPGASVKVSCKASGYTFT | 2757 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] RVKFSRSADAPAYQ | |
| | QGQNQLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| CAT-CD70-299 | MALPVTALLLPLALLLHAARPQVQLVQSGAEVKKPGASVKVSCKASGYTFT | 2759 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] RVKFSRSADAPAYQ | |
| | QGQNQLYNELNLGRREEYDVLDKRRGRDPEMGGKPRRKNPQEGLYNELQKD | |
| | i | |
| CAT-CD 70-300 | MALPVTALLLPLALLLHAARPQVQLVQSGAEVKKPGASVKVSCKASGYTFT | 2761 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDIYIWAPLAGTCGVLLLSLVITLYCNHRN [symbol] | |
| CD3z signaling domain | [symbol] [symbol] RVKFSRSADAPAYQQGQNQLYNELNLG | |
| | RREEYDVLDKRRGRDPEMGGKPRRKNPQEGLYNELQKDKMAEAYSEIGMKG | |
| | ERRRGKGHDGLYQGLSTATKDTYDALHMQALPPR | |
| CAT-CD 70-301 | MALPVTALLLPLALLLHAARPQVQLVQSGAEVKKPGASVKVSCKASGYTFT | 2763 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKAEPKSPDKTHTCPPCPKDPIYIWAPLAGTCGVLLLSLVIT | |
| [symbol] CD3z | LYCNHRN [symbol] [symbol] RV | |
| signaling domain | KFSRSADAPAYQQGQNQLYNELNLGRREEYDVLDKRRGRDPEMGGKPRRKN | |
| | PQEGLYNELQKDKMAEAYSEIGMKGERRRGKGHDGLYQGLSTATKDTYDAL | |
| | HMQALPPR | |
| CAT-CD 70-302 | MALPVTALLLPLALLLHAARPQVQLVQSGAEVKKPGASVKVSCKASGYTFT | 2765 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPIYIWAPLAGTCGVLLLSLVITLYCNHRN | |
| [symbol] CD3z | [symbol] [symbol] RVKFSRSAD | |
| signaling domain | APAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALPP | |
| | R | |
| CAT-CD 70-303 | MALPVTALLLPLALLLHAARPQVQLVQSGAEVKKPGASVKVSCKASGYTFT | 2767 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKESKYGPPCPSCPGQPREPQVYTLPPSQEEMTKNQVSLTCL | |
| [symbol] CD3z | VKGFYPSDIAVEWESNGQPENNYKTTPPVLDSDGSFFLYSRLTVDKSRWQE | |
| signaling domain | GNVFSCSVMHEALHNHYTQKSLSLSLGKIYIWAPLAGTCGVLLLSLVITLY | |
| | CNHRN [symbol] [symbol] [symbol] RVKF | |
| | SRSADAPAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTYDALHM | |
| | QALPPR | |
| CAT-CD 70-304 | MALPVTALLLPLALLLHAARPQVQLVQSGAEVKKPGASVKVSCKASGYTFT | 2769 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] RVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATK | |
| | DTYDALHMQALPPR | |
| CAT-CD 70-305 | MALPVTALLLPLALLLHAARPQVQLVQSGAEVKKPGASVKVSCKASGYTFT | 2771 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] RVKFSRSADAPAYQQGQNQLYNELNLGRREEYDVLDKRRGRDPEMGG | |
| | KPRRKNPQEGLYNELQKDKMAEAYSEIGMKGERRRGKGHDGLYQGLSTATK | |
| | DTYDALHMQALPPR | |
| CAT-CD 70-306 | MALPVTALLLPLALLLHAARPQVQLVQSGAEVKKPGASVKVSCKASGYTFT | 2773 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDFWVLVVVGGVLACYSLLVTVAFIIFWV [symbol] | |
| DAP12 signaling | [symbol] YFLGRLVPRGRGAAEAATRKQRITETESP | |
| domain | YQELQGQRSDVYSDLNTQRPYYK | |
| CAT-CD 70-307 | MALPVTALLLPLALLLHAARPQVQLVQSGAEVKKPGASVKVSCKASGYTFT | 2775 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKAEPKSPDKTHTCPPCPKDPFWVLVVVGGVLACYSLLVTVA | |
| [symbol] DAP12 | FIIFWV [symbol] [symbol] YFLG | |
| signaling domain | RLVPRGRGAAEAATRKQRITETESPYQELQGQRSDVYSDLNTQRPYYK | |
| CAT-CD 70-308 | MALPVTALLLPLALLLHAARPQVQLVQSGAEVKKPGASVKVSCKASGYTFT | 2777 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPFWVLVVVGGVLACYSLLVTVAFIIFWV [symbol] | |
| [symbol] DAP12 | [symbol] [symbol] YFLGRLVPRGR | |
| signaling domain | GAAEAATRKQRITETESPYQELQGQRSDVYSDLNTQRPYYK | |
| CAT-CD 70-309 | MALPVTALLLPLALLLHAARPQVQLVQSGAEVKKPGASVKVSCKASGYTFT | 2779 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPGQPREPQVYTLPPSQEEMTKNQVSLTCL | |
| [symbol] DAP12 | VKGFYPSDIAVEWESNGQPENNYKTTPPVLDSDGSFFLYSRLTVDKSRWQE | |
| signaling domain | GNVFSCSVMHEALHNHYTQKSLSLSLGKFWVLVVVGGVLACYSLLVTVAFI | |
| | IFWV [symbol] [symbol] YFLGRL | |
| | VPRGRGAAEAATRKQRITETESPYQELQGQRSDVYSDLNTQRPYYK | |
| CAT-CD 70-310 | MALPVTALLLPLALLLHAARPQVQLVQSGAEVKKPGASVKVSCKASGYTFT | 2781 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TEGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| DAP12 signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKFWVLVVVGGVLACYS | |
| | LLVTVAFIIFWV [symbol] | |
| | [symbol] YFLGRLVPRGRGAAEAATRKQRITETESPYQELQGQRSDVYSDLNTQRP | |
| | YYK | |
| CAT-CD70-311 | MALPVTALLLPLALLLHAARPQVQLVQSGAEVKKPGASVKVSCKASGYTFT | 2783 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TEGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| DAP12 signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKFWVLVVVGGVLACYS | |
| | LLVTVAFIIFWV [symbol] [symbol] | |
| | [symbol] YFLGRLVPRGRGAAEAATRKQRITETESPYQELQGQRSDVYSDLNTQRP | |
| | YYK | |
| CAT-CD 70-312 | MALPVTALLLPLALLLHAARPQVQLVQSGAEVKKPGASVKVSCKASGYTFT | 2785 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDFWVLVVVGGVLACYSLLVTVAFIIFWV [symbol] | |
| CD3z signaling domain | [symbol] RVKFSRSADAPAYQQGQNQLYNELNLGRR | |
| | EEYDVLDKRRGRDPEMGGKPRRKNPQEGLYNELQKDKMAEAYSEIGMKGER | |
| | RRGKGHDGLYQGLSTATKDTYDALHMQALPPR | |
| CAT-CD70-360 | MALPVTALLLPLALLLHAARPQVQLVQSGAEVKKPGASVKVSCKASGYTFT | 2787 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKAEPKSPDKTHTCPPCPKDPFWVLVVVGGVLACYSLLVTVA | |
| [symbol] CD3z | FIIFWV [symbol] [symbol] RVKF | |
| signaling domain | SRSADAPAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTYDALHM | |
| | QALPPR | |
| CAT-CD 70-313 | MALPVTALLLPLALLLHAARPQVQLVQSGAEVKKPGASVKVSCKASGYTFT | 2789 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPFWVLVVVGGVLACYSLLVTVAFIIFWV [symbol] | |
| [symbol] CD3z | [symbol] [symbol] RVKFSRSADAP | |
| signaling domain | AYQQGQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQALPPR | |
| CAT-CD 70-314 | MALPVTALLLPLALLLHAARPQVQLVQSGAEVKKPGASVKVSCKASGYTFT | 2791 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPGQPREPQVYTLPPSQEEMTKNQVSLTCL | |
| [symbol] CD3z | VKGFYPSDIAVEWESNGQPENNYKTTPPVLDSDGSFFLYSRLTVDKSRWQE | |
| signaling domain | GNVFSCSVMHEALHNHYTQKSLSLSLGKFWVLVVVGGVLACYSLLVTVAFI | |
| | IFWV [symbol] [symbol] RVKFSR | |
| | SADAPAYQQGQNQLYNELNLGRREEYDVLDKRRGRDPEMGGKPRRKNPQEG | |
| | LYNELQKDKMAEAYSEIGMKGERRRGKGHDGLYQGLSTATKDTYDALHMQA | |
| | LPPR | |
| CAT-CD 70-315 | MALPVTALLLPLALLLHAARPQVQLVQSGAEVKKPGASVKVSCKASGYTFT | 2793 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKFWVLVVVGGVLACYS | |
| | LLVTVAFIIFWV [symbol] [symbol] | |
| | [symbol] RVKFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTATKDT | |
| | YDALHMQALPPR | |
| CAT-CD 70-316 | MALPVTALLLPLALLLHAARPQVQLVQSGAEVKKPGASVKVSCKASGYTFT | 2795 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKFWVLVVVGGVLACYS | |
| | LLVTVAFIIFWV [symbol] [symbol] | |
| | [symbol] RVKFSRSADAPAYQQGQNQLYNELNLGRREEYDVLDKRRGRDPEMGGKP | |
| | RRKNPQEGLYNELQKDKMAEAYSEIGMKGERRRGKGHDGLYQGLSTATKDT | |
| | YDALHMQALPPR | |
| CAT-CD 70-317 | MALPVTALLLPLALLLHAARPQVQLVQSGAEVKKPGASVKVSCKASGYTFT | 2797 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDFWVLVVVGGVLACYSLLVTVAFIIFWV [symbol] | |
| OX40L signaling | [symbol] ERVQPLEENVGNAARPRFERNKRVKFSRS | |
| domain, CD3z | ADAPAYQQGQNQLYNELNLGRREEYDVLDKRRGRDPEMGGKPRRKNPQEGL | |
| signaling domain | YNELQKDKMAEAYSEIGMKGERRRGKGHDGLYQGLSTATKDTYDALHMQAL | |
| | PPR | |
| CAT-CD 70-318 | MALPVTALLLPLALLLHAARPQVQLVQSGAEVKKPGASVKVSCKASGYTFT | 2799 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKFVPVFLPAKPTTTPAPRPPTPAPTIASQPLSLRPEACRPA | |
| [symbol] OX40L | AGGAVHTRGLDFACDFWVLVVVGGVLACYSLLVTVAFIIFWV [symbol] | |
| signaling domain, | [symbol] [symbol] ERVQPLEENVGNAARPRFE | |
| CD3z signaling domain | RNKRVKFSRSADAPAYQQGQNQLYNELNLGRREEYDVLDKRRGRDPEMGGK | |
| | PRRKNPQEGLYNELQKDKMAEAYSEIGMKGERRRGKGHDGLYQGLSTATKD | |
| | TYDALHMQALPPR | |
| CAT-CD 70-319 | MALPVTALLLPLALLLHAARPQVQLVQSGAEVKKPGASVKVSCKASGYTFT | 2801 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKAEPKSPDKTHTCPPCPKDPFWVLVVVGGVLACYSLLVTVA | |
| [symbol] OX40L | FIIFWV [symbol] [symbol] ERVQ | |
| signaling domain, | PLEENVGNAARPRFERNKRVKFSRSADAPAYQQGQNQLYNELNLGRREEYD | |
| CD3z signaling domain | VLDKRRGRDPEMGGKPRRKNPQEGLYNELQKDKMAEAYSEIGMKGERRRGK | |
| | GHDGLYQGLSTATKDTYDALHMQALPPR | |
| CAT-CD 70-320 | MALPVTALLLPLALLLHAARPQVQLVQSGAEVKKPGASVKVSCKASGYTFT | 2803 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPFWVLVVVGGVLACYSLLVTVAFIIFWV [symbol] | |
| [symbol] OX40L | [symbol] [symbol] ERVQPLEENVG | |
| signaling domain, | NAARPRFERNKRVKFSRSADAPAYQQGQNQLYNELNLGRREEYDVLDKRRG | |
| CD3z signaling domain | RDPEMGGKPRRKNPQEGLYNELQKDKMAEAYSEIGMKGERRRGKGHDGLYQ | |
| | GLSTATKDTYDALHMQALPPR | |
| CAT-CD70-321 | MALPVTALLLPLALLLHAARPQVQLVQSGAEVKKPGASVKVSCKASGYTFT | 2805 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKESKYGPPCPSCPGQPREPQVYTLPPSQEEMTKNQVSLTCL | |
| [symbol] OX40L | VKGFYPSDIAVEWESNGQPENNYKTTPPVLDSDGSFFLYSRLTVDKSRWQE | |
| signaling domain, | GNVFSCSVMHEALHNHYTQKSLSLSLGKFWVLVVVGGVLACYSLLVTVAFI | |
| CD3z signaling domain | IFWV [symbol] [symbol] ERVQPL | |
| | EENVGNAARPRFERNKRVKFSRSADAPAYQQGQNQLYNELNLGRREEYDVL | |
| | DKRRGRDPEMGGKPRRKNPQEGLYNELQKDKMAEAYSEIGMKGERRRGKGH | |
| | DGLYQGLSTATKDTYDALHMQALPPR | |
| CAT-CD 70-322 | MALPVTALLLPLALLLHAARPQVQLVQSGAEVKKPGASVKVSCKASGYTFT | 2807 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| OX40L signaling | HQDWLNGKEYKCKVSNKGLPSSIEKTISKAKGQPREPQVYTLPPSQEEMTK | |
| domain, CD3z | NQVSLTCLVKGFYPSDIAVEWESNGQPENNYKTTPPVLDSDGSFFLYSRLT | |
| signaling domain | VDKSRWQEGNVFSCSVMHEALHNHYTQKSLSLSLGKFWVLVVVGGVLACYS | |
| | LLVTVAFIIFWV [symbol] [symbol] | |
| | [symbol] ERVQPLEENVGNAARPRFERNKRVKFSRSADAPAYQQGQNQLYNELNLG | |
| | RREEYDVLDKRRGRDPEMGGKPRRKNPQEGLYNELQKDKMAEAYSEIGMKG | |
| | ERRRGKGHDGLYQGLSTATKDTYDALHMQALPPR | |
| CAT-CD 70-323 | MALPVTALLLPLALLLHAARPQVQLVQSGAEVKKPGASVKVSCKASGYTFT | 2809 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| OX40L signaling | HQDWLNGKEYKCKVSNKGLPSSIEKTISKAKGQPREPQVYTLPPSQEEMTK | |
| domain, CD3z | NQVSLTCLVKGFYPSDIAVEWESNGQPENNYKTTPPVLDSDGSFFLYSRLT | |
| signaling domain | VDKSRWQEGNVFSCSVMHEALHNHYTQKSLSLSLGKFWVLVVVGGVLACYS | |
| | LLVTVAFIIFWV [symbol] [symbol] | |
| | [symbol] ERVQPLEENVGNAARPRFERNKRVKFSRSADAPAYQQGQNQLYNELNLG | |
| | RREEYDVLDKRRGRDPEMGGKPRRKNPQEGLYNELQKDKMAEAYSEIGMKG | |
| | ERRRGKGHDGLYQGLSTATKDTYDALHMQALPPR | |
| CAT-CD 70-324 | MALPVTALLLPLALLLHAARPQVQLVQSGAEVKKPGASVKVSCKASGYTFT | 2811 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] CD3z | DFACDIYIWAPLAGTCGVLLLSLVITLYCNHRN [symbol] | |
| signaling domain | [symbol] [symbol] [symbol] | |
| | [symbol] [symbol] [symbol] | |
| | RVKFSRSADAPAYQQGQNQLYNELNLGRREEYDVLDKRRGRDPEMGGKPRR | |
| | KIVPQEGLYNELQKDKMAEAYSEIGMKGERRRGKGHDGLYQGLSTATKDTYD | |
| | ALHMQALPPR | |
| CAT-CD 70-325 | MALPVTALLLPLALLLHAARPQVQLVQSGAEVKKPGASVKVSCKASGYTFT | 2813 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKAEPKSPDKTHTCPPCPKDPIYIWAPLAGTCGVLLLSLVIT | |
| CD3z signaling domain | LYCNHRN [symbol] [symbol] | |
| | [symbol] [symbol] [symbol] | |
| | [symbol] RVKFSRSADAPAYQQGQNQLYNELNL | |
| | GRREEYDVLDKRRGRDPEMGGKPRRKNPQEGLYNELQKDKMAEAYSEIGMK | |
| | GERRRGKGHDGLYQGLSTATKDTYDALHMQALPPR | |
| CAT-CD70-326 | MALPVTALLLPLALLLHAARPQVQLVQSGAEVKKPGASVKVSCKASGYTFT | 2815 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPIYIWAPLAGTCGVLLLSLVITLYCNHRN | |
| CD3z signaling domain | [symbol] [symbol] [symbol] | |
| | [symbol] [symbol] [symbol] | |
| | [symbol] RVKFSRSADAPAYQQGQNQLYNELNLGRREEYD | |
| | VLDKRRGRDPEMGGKPRRKNPQEGLYNELQKDKMAEAYSEIGMKGERRRGK | |
| | GHDGLYQGLSTATKDTYDALHMQALPPR | |
| CAT-CD70-327 | MALPVTALLLPLALLLHAARPQVQLVQSGAEVKKPGASVKVSCKASGYTFT | 2817 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKESKYGPPCPSCPGQPREPQVYTLPPSQEEMTKNQVSLTCL | |
| CD3z signaling domain | VKGFYPSDIAVEWESNGQPENNYKTTPPVLDSDGSFFLYSRLTVDKSRWQE | |
| | GNVFSCSVMHEALHNHYTQKSLSLSLGKIYIWAPLAGTCGVLLLSLVITLY | |
| | CNHRN [symbol] [symbol] | |
| | [symbol] [symbol] [symbol] | |
| | [symbol] RVKFSRSADAPAYQQGQNQLYNELNLGR | |
| | REEYDVLDKRRGRDPEMGGKPRRKNPQEGLYNELQKDKMAEAYSEIGMKGE | |
| | RRRGKGHDGLYQGLSTATKDTYDALHMQALPPR | |
| CAT-CD70-328 | MALPVTALLLPLALLLHAARPQVQLVQSGAEVKKPGASVKVSCKASGYTFT | 2819 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| CD3z | VTCVVVDVSQEDPEVQFNWYVDGVEVHNAKTKPREEQFNSTYRVVSVLTVL | |
| signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] [symbol] [symbol] | |
| | [symbol] [symbol] RVKFSRSADAPAYQQGQNQL | |
| | YNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| CAT-CD70-329 | MALPVTALLLPLALLLHAARPQVQLVQSGAEVKKPGASVKVSCKASGYTFT | 2821 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| CD3z | VTCVVVDVSQEDPEVQFNWYVDGVEVHNAKTKPREEQFQSTYRVVSVLTVL | |
| signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] [symbol] [symbol] | |
| | [symbol] [symbol] RVKFSRSADAPAYQQGQNQL | |
| | YNELNLGRREEYDVLDKRRGRDPEMGGKPRRKNPQEGLYNELQKDKMAEAY | |
| | SEIGMKGERRRGKGHDGLYQGLSTATKDTYDALHMQALPPR | |
| CAT-CD70-330 | MALPVTALLLPLALLLHAARPQVQLVQSGAEVKKPGASVKVSCKASGYTFT | 2823 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDIYIWAPLAGTCGVLLLSLVITLYCNHRN [symbol] | |
| CD3z signaling domain | [symbol] [symbol] RVKFSRSADAPAYQQGQ | |
| | NQLYNELNLGRREEYDVLDKRRGRDPEMGGKPRRKNPQEGLYNELQKDKMA | |
| | EAYSEIGMKGERRRGKGHDGLYQGLSTATKDTYDALHMQALPPR | |
| CAT-CD70-331 | MALPVTALLLPLALLLHAARPQVQLVQSGAEVKKPGASVKVSCKASGYTFT | 2825 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKAEPKSPDKTHTCPPCPKDPIYIWAPLAGTCGVLLLSLVIT | |
| [symbol] CD3z | LYCNHRN [symbol] [symbol] | |
| signaling domain | [symbol] RVKFSRSADAPAYQQGQNQLYNELNLGRREEYDVLDKRRGRDP | |
| | EMGGKPRRKNPQEGLYNELQKDKMAEAYSEIGMKGERRRGKGHDGLYQGLS | |
| | TATKDTYDALHMQALPPR | |
| CAT-CD70-3 3 2 | MALPVTALLLPLALLLHAARPQVQLVQSGAEVKKPGASVKVSCKASGYTFT | 2827 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPIYIWAPLAGTCGVLLLSLVITLYCNHRN | |
| [symbol] CD3z | [symbol] [symbol] [symbol] | |
| signaling domain | [symbol] RVKFSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTATKDTY | |
| | DALHMQALPPR | |
| CAT-CD70-333 | MALPVTALLLPLALLLHAARPQVQLVQSGAEVKKPGASVKVSCKASGYTFT | 2829 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKESKYGPPCPSCPGQPREPQVYTLPPSQEEMTKNQVSLTCL | |
| [symbol] CD3z | VKGFYPSDIAVEWESNGQPENNYKTTPPVLDSDGSFFLYSRLTVDKSRWQE | |
| signaling domain | GNVFSCSVMHEALHNHYTQKSLSLSLGKIYIWAPLAGTCGVLLLSLVITLY | |
| | CNHRN [symbol] [symbol] [symbol] | |
| | [symbol] RVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQGLSTA | |
| | TKDTYDALHMQALPPR | |
| CAT-CD70-334 | MALPVTALLLPLALLLHAARPQVQLVQSGAEVKKPGASVKVSCKASGYTFT | 2831 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] RVKFSRSADAPAYQQGQNQLYNELNLGRREEYDVLDK | |
| | RRGRDPEMGGKPRRKNPQEGLYNELQKDKMAEAYSEIGMKGERRRGKGHDG | |
| | LYQGLSTATKDTYDALHMQALPPR | |
| CAT-CD70-335 | MALPVTALLLPLALLLHAARPQVQLVQSGAEVKKPGASVKVSCKASGYTFT | 2833 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| CD3z signaling domain | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] RVKFSRSADAPAYQQGQNQLYNELNLGRREEYDVLDK | |
| | RRGRDPEMGGKPRRKNPQEGLYNELQKDKMAEAYSEIGMKGERRRGKGHDG | |
| | LYQGLSTATKDTYDALHMQALPPR | |
| CAT-CD70-336 | MALPVTALLLPLALLLHAARPQVQLVQSGAEVKKPGASVKVSCKASGYTFT | 2835 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, CD3z | TFGQGTKVEIKTTTPAPRPPTPAPTIASQPLSLRPEACRPAAGGAVHTRGL | |
| signaling domain | DFACDIYIWAPLAGTCGVLLLSLVITLYCNHRNRVKFSRSADAPAYQQGQN | |
| | QLYNELNLGRREEYDVLDKRRGRDPEMGGKPRRKNPQEGLYNELQKDKMAE | |
| | AYSEIGMKGERRRGKGHDGLYQGLSTATKDTYDALHMQALPPR | |
| CAT-CD70-337 | MALPVTALLLPLALLLHAARPQVQLVQSGAEVKKPGASVKVSCKASGYTFT | 2837 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| CD3z signaling domain | TEGQGTKVEIKAEPKSPDKTHTCPPCPKDPIYIWAPLAGTCGVLLLSLVIT | |
| | LYCNHRNRVKFSRSADAPAYQQGQNQLYNELNLGRREEYDVLDKRRGRDPE | |
| | MGGKPRRKNPQEGLYNELQKDKMAEAYSEIGMKGERRRGKGHDGLYQGLST | |
| | ATKDTYDALHMQALPPR | |
| CAT-CD70-338 | MALPVTALLLPLALLLHAARPQVQLVQSGAEVKKPGASVKVSCKASGYTFT | 2839 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| CD3z signaling domain | TFGQGTKVEIKESKYGPPCPSCPIYIWAPLAGTCGVLLLSLVITLYCNHRN | |
| | RVKFSRSADAPAYQQGQNQLYNELNLGRREEYDVLDKRRGRDPEMGGKPRR | |
| | KIVPQEGLYNELQKDKMAEAYSEIGMKGERRRGKGHDGLYQGLSTATKDTYD | |
| | ALHMQALPPR | |
| CAT-CD70-339 | MALPVTALLLPLALLLHAARPQVQLVQSGAEVKKPGASVKVSCKASGYTFT | 2841 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| CD3z signaling domain | TEGQGTKVEIKESKYGPPCPSCPGQPREPQVYTLPPSQEEMTKNQVSLTCL | |
| | VKGFYPSDIAVEWESNGQPENNYKTTPPVLDSDGSFFLYSRLTVDKSRWQE | |
| | GNVFSCSVMHEALHNHYTQKSLSLSLGKIYIWAPLAGTCGVLLLSLVITLY | |
| | CNHRNRVKFSRSADAPAYQQGQNQLYNELNLGRREEYDVLDKRRGRDPEMG | |
| | GKPRRKNPQEGLYNELQKDKMAEAYSEIGMKGERRRGKGHDGLYQGLSTAT | |
| | KDTYDALHMQALPPR | |
| CAT-CD70-340 | MALPVTALLLPLALLLHAARPQVQLVQSGAEVKKPGASVKVSCKASGYTFT | 2843 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, CD3z | TFGQGTKVEIKESKYGPPCPSCPAPEFLGGPSVFLFPPKPKDTLMISRTPE | |
| signaling domain | VTCVVVDVSQEDPEVQFNWYVDGVEVHNAKTKPREEQFNSTYRVVSVLTVL | |
| | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRNRVKFSRSADAPAYQQGQNQLYNELNLGRREEYDVLDKR | |
| | RGRDPEMGGKPRRKNPQEGLYNELQKDKMAEAYSEIGMKGERRRGKGHDGL | |
| | YQGLSTATKDTYDALHMQALPPR | |
| CAT-CD70-341 | MALPVTALLLPLALLLHAARPQVQLVQSGAEVKKPGASVKVSCKASGYTFT | 2845 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, CD3z | TFGQGTKVEIKESKYGPPCPSCPAPEFEGGPSVFLFPPKPKDTLMISRTPE | |
| signaling domain | VTCVVVDVSQEDPEVQFNWYVDGVEVHNAKTKPREEQFQSTYRVVSVLTVL | |
| | HQDWLNGKEYKCKVSNKGLPSSIEKTISKAKGQPREPQVYTLPPSQEEMTK | |
| | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRNRVKFSRSADAPAYQQGQNQLYNELNLGRREEYDVLDKR | |
| | RGRDPEMGGKPRRKNPQEGLYNELQKDKMAEAYSEIGMKGERRRGKGHDGL | |
| | YQGLSTATKDTYDALHMQALPPR | |
| CAT-CD70-342 | MALPVTALLLPLALLLHAARPQVQLVQSGAEVKKPGASVKVSCKASGYTFT | 2847 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDIYIWAPLAGTCGVLLLSLVITLYCNHRN [symbol] | |
| DAP12 signaling | [symbol] YFLGRLVPRGRGAAEAATRKQRITET | |
| domain | ESPYQELQGQRSDVYSDLNTQRPYYK | |
| CAT-CD70-343 | MALPVTALLLPLALLLHAARPQVQLVQSGAEVKKPGASVKVSCKASGYTFT | 2849 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKAEPKSPDKTHTCPPCPKDPIYIWAPLAGTCGVLLLSLVIT | |
| [symbol] DAP12 | LYCNHRN [symbol] [symbol] Y | |
| signaling domain | FLGRLVPRGRGAAEAATRKQRITETESPYQELQGQRSDVYSDLNTQRPYYK | |
| CAT-CD70-344 | MALPVTALLLPLALLLHAARPQVQLVQSGAEVKKPGASVKVSCKASGYTFT | 2851 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPIYIWAPLAGTCGVLLLSLVITLYCNHRN | |
| [symbol] DAP12 | [symbol] [symbol] YFLGRLVP | |
| signaling domain | RGRGAAEAATRKQRITETESPYQELQGQRSDVYSDLNTQRPYYK | |
| CAT-CD70-345 | MALPVTALLLPLALLLHAARPQVQLVQSGAEVKKPGASVKVSCKASGYTFT | 2853 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKESKYGPPCPSCPGQPREPQVYTLPPSQEEMTKNQVSLTCL | |
| [symbol] DAP12 | VKGFYPSDIAVEWESNGQPENNYKTTPPVLDSDGSFFLYSRLTVDKSRWQE | |
| signaling domain | GNVFSCSVMHEALHNHYTQKSLSLSLGKIYIWAPLAGTCGVLLLSLVITLY | |
| | CNHRN [symbol] [symbol] YFL | |
| | GRLVPRGRGAAEAATRKQRITETESPYQELQGQRSDVYSDLNTQRPYYK | |
| CAT-CD70-346 | MALPVTALLLPLALLLHAARPQVQLVQSGAEVKKPGASVKVSCKASGYTFT | 2855 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| DAP12 signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] YFLGRLVPRGRGAAEAATRKQRITETESPYQELQGQRSDVYSDLNT | |
| | QRPYYK | |
| CAT-CD70-347 | MALPVTALLLPLALLLHAARPQVQLVQSGAEVKKPGASVKVSCKASGYTFT | 2857 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| DAP12 signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] YFLGRLVPRGRGAAEAATRKQRITETESPYQELQGQRSDVYSDLNT | |
| | QRPYYK | |
| CAT-CD70-348 | MALPVTALLLPLALLLHAARPQVQLVQSGAEVKKPGASVKVSCKASGYTFT | 2859 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDIYIWAPLAGTCGVLLLSLVITLYCNHRN [symbol] | |
| DAP12 signaling | [symbol] [symbol] YFLGRLVPRGRGAAEAATRKQRITETE | |
| domain | SPYQELQGQRSDVYSDLNTQRPYYK | |
| CAT-CD70-349 | MALPVTALLLPLALLLHAARPQVQLVQSGAEVKKPGASVKVSCKASGYTFT | 2861 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKAEPKSPDKTHTCPPCPKDPIYIWAPLAGTCGVLLLSLVIT | |
| [symbol] DAP12 | LYCNHRN [symbol] [symbol] YF | |
| signaling domain | LGRLVPRGRGAAEAATRKQRITETESPYQELQGQRSDVYSDLNTQRPYYK | |
| CAT-CD70-350 | MALPVTALLLPLALLLHAARPQVQLVQSGAEVKKPGASVKVSCKASGYTFT | 2863 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPIYIWAPLAGTCGVLLLSLVITLYCNHRN | |
| [symbol] DAP12 | [symbol] [symbol] YFLGRLVPR | |
| signaling domain | GRGAAEAATRKQRITETESPYQELQGQRSDVYSDLNTQRPYYK | |
| CAT-CD70-351 | MALPVTALLLPLALLLHAARPQVQLVQSGAEVKKPGASVKVSCKASGYTFT | 2865 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD8α | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TEGQGTKVEIKESKYGPPCPSCPGQPREPQVYTLPPSQEEMTKNQVSLTCL | |
| [symbol] DAP12 | VKGFYPSDIAVEWESNGQPENNYKTTPPVLDSDGSFFLYSRLTVDKSRWQE | |
| signaling domain | GNVFSCSVMHEALHNHYTQKSLSLSLGKIYIWAPLAGTCGVLLLSLVITLY | |
| | CNHRN [symbol] [symbol] YFLG | |
| | RLVPRGRGAAEAATRKQRITETESPYQELQGQRSDVYSDLNTQRPYYK | |
| CAT-CD70-352 | MALPVTALLLPLALLLHAARPQVQLVQSGAEVKKPGASVKVSCKASGYTFT | 2867 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| DAP12 signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] YFLGRLVPRGRGAAEAATRKQRITETESPYQELQGQRSDVYSDLNTQ | |
| | RPYYK | |
| CAT-CD70-353 | MALPVTALLLPLALLLHAARPQVQLVQSGAEVKKPGASVKVSCKASGYTFT | 2869 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD8α transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| DAP12 signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKIYIWAPLAGTCGVLL | |
| | LSLVITLYCNHRN [symbol] [symbol] | |
| | [symbol] YFLGRLVPRGRGAAEAATRKQRITETESPYQELQGQRSDVYSDLNTQ | |
| | RPYYK | |
| CAT-CD70-354 | MALPVTALLLPLALLLHAARPQVQLVQSGAEVKKPGASVKVSCKASGYTFT | 2871 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| CD8α short hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKTTTPAPRPPTPAPTIASQPLSLRPEACRPAAGGAVHTRGL | |
| [symbol] | DFACDFWVLVVVGGVLACYSLLVTVAFIIFWV [symbol] | |
| FCER1G signaling | [symbol] RLKIQVRKAAITSYEKSDGVYTGLSTRNQ | |
| domain | ETYETLKHEKPPQ | |
| CAT-CD70-355 | MALPVTALLLPLALLLHAARPQVQLVQSGAEVKKPGASVKVSCKASGYTFT | 2873 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG1 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKAEPKSPDKTHTCPPCPKDPFWVLVVVGGVLACYSLLVTVA | |
| [symbol] FCER1G | FIIFWV [symbol] [symbol] RLKI | |
| signaling domain | QVRKAAITSYEKSDGVYTGLSTRNQETYETLKHEKPPQ | |
| CAT-CD70-356 | MALPVTALLLPLALLLHAARPQVQLVQSGAEVKKPGASVKVSCKASGYTFT | 2875 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 short hinge, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPFWVLVVVGGVLACYSLLVTVAFIIFWV [symbol] | |
| [symbol] FCER1G | [symbol] [symbol] RLKIQVRKAAI | |
| signaling domain | TSYEKSDGVYTGLSTRNQETYETLKHEKPPQ | |
| CAT-CD70-357 | MALPVTALLLPLALLLHAARPQVQLVQSGAEVKKPGASVKVSCKASGYTFT | 2877 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH3, CD28 | SGDIVMTQSPDSLAVSLGERATINCRASKSVSTSGYSFMHWYQQKPGQPPK | |
| transmembrane domain, | LLIYLASNLESGVPDRFSGSGSGTDFTLTISSLQAEDVAVYYCQHSREVPW | |
| [symbol] | TFGQGTKVEIKESKYGPPCPSCPGQPREPQVYTLPPSQEEMTKNQVSLTCL | |
| [symbol] FCER1G | VKGFYPSDIAVEWESNGQPENNYKTTPPVLDSDGSFFLYSRLTVDKSRWQE | |
| signaling domain | GNVFSCSVMHEALHNHYTQKSLSLSLGKFWVLVVVGGVLACYSLLVTVAFI | |
| | IFWV [symbol] [symbol] RLKIQV | |
| | RKAAITSYEKSDGVYTGLSTRNQETYETLKHEKPPQ | |
| CAT-CD70-358 | MALPVTALLLPLALLLHAARPQVQLVQSGAEVKKPGASVKVSCKASGYTFT | 2879 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 hinge-CH2-CH3, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFLGGPSVFLFPPKPKDTLMISRTPE | |
| [symbol] | VTCVVVDVSQEDPEVQFNWYVDGVEVHNAKTKPREEQFNSTYRVVSVLTVL | |
| FCER1G signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKFWVLVVVGGVLACYS | |
| | LLVTVAFIIFWV [symbol] [symbol] | |
| | [symbol] RLKIQVRKAAITSYEKSDGVYTGLSTRNQETYETLKHEKPPQ | |
| CAT-CD 70-359 | MALPVTALLLPLALLLHAARPQVQLVQSGAEVKKPGASVKVSCKASGYTFT | 2881 |
| CD8α signal peptide, | NYGMNWVRQAPGQGLKWMGWINTYTGEPTYADAFKGRVTMTRDTSISTAYM | |
| CD70 scFv (1F6), | ELSRLRSDDTAVYYCARDYGDYGMDYWGQGTTVTVSSGGGGSGGGGSGGGG | |
| IgG4 mutant hinge, | SGDIVMTQSPDSLAVSLGERATINCRASKSVSTSGYSFMHWYQQKPGQPPK | |
| CD28 transmembrane | LLIYLASNLESGVPDRFSGSGSGTDFTLTISSLQAEDVAVYYCQHSREVPW | |
| domain, [symbol] | TFGQGTKVEIKESKYGPPCPSCPAPEFEGGPSVFLFPPKPKDTLMISRTPE | |
| [symbol] | VTCVVVDVSQEDPEVQFNWYVDGVEVHNAKTKPREEQFQSTYRVVSVLTVL | |
| FCER1G signaling | HQDWLNGKEYKCKVSNKGLPSSIEKTISKAKGQPREPQVYTLPPSQEEMTK | |
| domain | NQVSLTCLVKGFYPSDIAVEWESNGQPENNYKTTPPVLDSDGSFFLYSRLT | |
| | VDKSRWQEGNVFSCSVMHEALHNHYTQKSLSLSLGKFWVLVVVGGVLACYS | |
| | LLVTVAFIIFWV [symbol] [symbol] | |
| | [symbol] RLKIQVRKAAITSYEKSDGVYTGLSTRNQETYETLKHEKPPQ | |

### 5. Functional Effector Elements

The present disclosure provides an NK cell or a population of NK cells engineered to express a chimeric antigen receptor (CAR), optionally, wherein the CAR comprises a) an antigen recognition domain, b) a hinge domain, c) a transmembrane domain, c) a costimulatory domain and e) an activation domain, and further engineered to express a functional effector element, such as, at least one exogenous polypeptide selected from the group of a cytokine (e.g., a membrane-bound cytokine), a chemokine, ligand, receptor, monoclonal antibody, bispecific T cell engager, peptide or enzyme, a TGFbeta signal converter, a TGFbeta decoy receptor, a safety switch protein, a subunit or a portion of the foregoing, or any combination of the foregoing).

Functional effector elements are any polypeptides that may improve the persistence, proliferation, or survival of an immune cell (e.g., NK cell) in a tumor microenvironment or improve the homing of the immune cell to the tumor. Functional effector elements may also improve the effector function (e.g., cytolysis or cytokine production) of an immune cell or enable an immune cell to overcome the immunosuppressive effects of the tumor microenvironment. In some embodiments, functional effector elements are soluble (e.g., secreted by the cell). In some embodiments, functional effector elements are membrane bound. Exemplary functional effector elements include, but are not limited to, cytokines, chemokine receptors, heparanase, a therapeutic agent, or any protein that overcomes immunosuppression of the tumor microenvironment.

In some embodiments, the NK cell or population of NK cells comprising a CAR described herein is administered to a subject with one or more additional therapeutic agents that include but are not limited to cytokines. In some embodiments, the NK cell or population of NK cells comprising a CAR, as provided herein, are engineered to express a functional effector element selected from a therapeutic agent, a cytokine, a chemokine receptor, or a protein that overcomes immunosuppression of the tumor microenvironment. In some embodiments, an NK cell or population of NK cells provided herein comprises (e.g., is modified to express) or is administered to a subject with at least one therapeutic agent selected from p40, LIGHT, CD40L, FLT3L, 4-1BBL, FASL, and haparanase. In some embodiments, an NK cell or population of NK cells provided herein comprises (e.g., is modified to express) or is administered to a subject with at least one cytokine, wherein the cytokine comprises at least one chemokine, interferon, interleukin, lymphokine, tumor necrosis factor, or variant or combination thereof. In some embodiments, the cytokine is an interleukin. In some embodiments, the interleukin is IL-15, IL-21, IL-2, IL-12, IL18, IL-21, IL-1, IL-3, IL-4, IL-5, IL-6, IL-7, IL-8, IL-9, IL-10, IL-11, IL-13, IL-14, IL-15, IL-16, IL-17, IL-19, IL-20, IL-22, IL-23, IL-24, IL-25, IL-26, IL-27, IL-28, IL-29, IL-30, IL-31, IL-32, IL-33, functional variants thereof, fragments thereof or combinations thereof.

In some embodiments, the cytokine is a soluble cytokine, a membrane-bound cytokine and/or a cytokine that is co-expressed with a cytokine receptor. In some embodiments, the membrane-bound cytokine is IL-21. In some embodiments, the membrane-bound cytokine is IL-18. In some embodiments, the membrane bound cytokine is IL-12. In some embodiments, the membrane bound cytokine is IL-15. In some embodiments, IL-21 is co-expressed with IL-21R. In some embodiments, IL-18 is co-expressed with IL-18Ra. In some embodiments, IL-12 is co-expressed with IL-12Rβ1. In some embodiments, IL-15 is co-expressed with IL-15Ra.

IL-12 plays an essential role in mediating the interaction of the innate and adaptive arms of the immune system, acting on T-cells and natural killer (NK) cells, enhancing the proliferation and activity of cytotoxic lymphocytes and the production of other inflammatory cytokines, especially interferon-gamma (IFN-gamma). IL-12 is a heterodimer of a 35-kD subunit (p35) and a 40-kD subunit (p40) linked through a disulfide linkage to make fully functional IL-12p70. The IL-12 gene encodes both the p35 and p40 subunits. Thus, in some embodiments, an NK cell or population of NK cells provided herein comprises (e.g., is modified to express), or is administered to a subject with, one or more of IL-12, membrane-bound IL-12, a fusion protein comprising IL12 subunits p35 and p40.

Interleukin-15 (IL-15) is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits AICD. NK cells expressing IL-15 are capable of continued supportive cytokine signaling, which is critical to their survival post-infusion. In some embodiments, an NK cell or population of NK cells provided herein comprises (e.g., is modified to express), or is administered to a subject with, at least one interleukin, wherein the interleukin comprises or consists of soluble or secreted IL-15, membrane bound IL-15 (mbIL-15), a IL-15 receptor alpha (mbIL-15Ra), a mbIL-15 with co-expressed IL-15Ra, a fusion of IL-15 and IL-15Ra, or a soluble IL-15 with co-expressed IL-15Ra. In some embodiments, the IL-15 is a soluble or secreted IL-15 that complexes with co-expressed IL15Ra on the NK cell or population of NK cells. Exemplary membrane bound IL-15 (mbIL-15) and fusion IL-15 and IL-15Ra are described in U.S. Pat. Nos. 10,428,305 and 9,629,877, each of which are incorporated herein by reference in their entirety. Exemplary membrane bound IL-15 are also described in Hurton et al. (2016) Proc. Nat'l. Acad. Sci. USA 113(48): E7788-97, incorporated herein by reference in its entirety.

The functional effector elements provided herein (also described as “exogenous stimulatory polypeptides” or “stimulatory polypeptides” herein) may comprise one or more linkers. For example, a linker may be disposed between two polypeptide sequences of the exogenous stimulatory polypeptide (e.g., between a cytokine polypeptide sequence and a transmembrane domain sequence, between two subunit sequences of an exogenous stimulatory polypeptide (e.g., between the p40 and p35 subunits of IL-12), or between two stimulatory polypeptides (e.g., IL-15 and IL-15RA)).

In some embodiments, the linker comprises or consists of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more amino acids in length. In some embodiments, the linker comprises or consists of between about 5 and about 25 amino acids in length, between about 5 and about 20 amino acids in length, between about 10 and about 25 amino acids in length, or between about 10 and about 20 amino acids in length. In some embodiments, the linker comprises or consists of 10, 11, 12, 13, 14, 15, 16, 17, 18, 19, or 20 amino acids in length. In a preferred embodiment, the linker is non-immunogenic.

In some embodiments, the linker comprises or consists of an amino acid sequence provided in Table 7.

In some embodiments, the linker comprises or consists of the amino acid sequence (GGGGS)n (SEQ ID NO: 665), wherein n is 1, 2, 3, 4, 5, 6, 7, 8, 9 or 10. In some embodiments, the linker comprises or consists of the amino acid sequence of SEQ ID NO: 652. In some embodiments, the linker comprises or consists of the amino acid sequence of SEQ ID NO: 653. In some embodiments, the linker comprises or consists of the amino acid sequence of SEQ ID NO: 654. In some embodiments, the linker comprises or consists of the amino acid sequence of SEQ ID NO: 655. In some embodiments, the linker comprises or consists of the amino acid sequence of SEQ ID NO: 654.

Other suitable linkers, which are known to one skilled in the art, may be used, e.g., to link an exogenous stimulatory polypeptide to a transmembrane domain, to link two exogenous stimulatory polypeptides (e.g., IL-15 and IL-15RA) or to link subunits of an exogenous stimulatory polypeptide (e.g., p30 and p40 of IL12). In certain embodiments, internal ribosome entry sites (IRES) elements are used to create multigene, or polycistronic messenger RNAs. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

2A sequence elements can be used to create linked- or co-expression of genes in the nucleic acid constructs provided in the present disclosure. For example, cleavage sequences could be used to co-express genes by linking open reading frames to form a single cistron. Exemplary cleavage sequences include but are not limited to T2A, P2A, E2A and F2A. In a preferred embodiment, the cleavage sequence comprises a P2A sequence.

In some embodiments, T2A comprises an amino acid sequence having at least 90%, 91%, 92%, 93%, 94%, 95%, 96%, 97%, 98%, 99% or 100% identity with the amino acid sequence of SEQ ID NO: 666.

In some embodiments, P2A comprises an amino acid sequence having at least 90%, 91%, 92%, 93%, 94%, 95%, 96%, 97%, 98%, 99% or 100% identity with the amino acid sequence of SEQ ID NO: 667.

In some embodiments, E2A comprises an amino acid sequence having at least 90%, 91%, 92%, 93%, 94%, 95%, 96%, 97%, 98%, 99% or 100% identity with the amino acid sequence of SEQ ID NO: 705.

In some embodiments, F2A comprises an amino acid sequence having at least 90%, 91%, 92%, 93%, 94%, 95%, 96%, 97%, 98%, 99% or 100% identity with the amino acid sequence of SEQ ID NO: 706.

In some embodiments, the cytokine is soluble IL-12. In some embodiments the cytokine is a membrane bound IL-12. In some cases, the IL-12p40 is indirectly linked to the IL-12p35 through a linker. In some embodiments, IL-12p40 and IL-12p35 are separated by an IRES sequence or a P2A sequence. In some embodiments, the cytokines described above can be under the control of an inducible promoter for gene transcription. In some embodiments, the inducible promoter is an EF1a promoter. In some embodiments, the inducible promoter is a PGK promoter.

In some embodiments, IL-12p40 comprises an amino acid sequence having at least 90%, 91%, 92%, 93%, 94%, 95%, 96%, 97%, 98%, 99% or 100% identity with the amino acid sequence of SEQ ID NO: 668.

In some embodiments, IL-12p35 comprises an amino acid sequence having at least 90%, 91%, 92%, 93%, 94%, 95%, 96%, 97%, 98%, 99% or 100% identity with the amino acid sequence of SEQ ID NO: 669.

An exemplary membrane bound IL-12 polypeptide “p40-(GS)15-IL15Ra(206-267)-P2A-p35” of the disclosure comprises or consists of the amino acid sequence of SEQ ID NO: 670. In some embodiments, the cytokine is soluble. In some embodiments the cytokine is membrane-bound. In some embodiments the cytokine is co-expressed with the cytokine receptor. In some embodiments, the cytokine is IL-15 or a fragment or variant thereof. In some embodiments the cytokine is a complex of IL-15 a fragment or variant thereof and a IL-15 Receptor alpha (IL-15Ra) or a fragment or variant thereof. In some embodiments, the IL-15 or a fragment or variant thereof and IL15Ra or fragment or variant thereof are expressed as a fusion polypeptide. In the case of the IL-15 fusion polypeptide, the IL-15 comprises a full-length IL-15 (e.g., a native IL-15 polypeptide) or fragment or variant thereof fused in frame with a full length IL-15Ra or functional fragment or variant thereof. In some cases, the IL-15 is linked to the IL-15Ra through a linker.

In some embodiments, the expression of any one of the functional effector elements provided herein (e.g., cytokines) can be under the control of an inducible promoter for gene transcription. In some embodiments, the inducible promoter is an EF1a promoter. In some embodiments, the inducible promoter is a PGK promoter.

In some embodiments, an NK cell or population of NK cells comprising (e.g., expressing) a CAR described herein also comprises (e.g., expresses) membrane-associated IL-15/IL-15RA. In some embodiments, an NK cell or population of NK cells comprising (e.g., expressing) a CAR described herein also comprises (e.g., expresses) mbIL-15 comprising a fusion protein between IL-15 and IL-15RA. In some embodiments, an NK cell or population of NK cells comprising (e.g., expressing) a CAR described herein also comprises (e.g., expresses) mbIL-15, wherein the mbIL-15 comprises, IL-15 and IL-15RA linked by a P2A sequence.

In some embodiments, the IL-15 comprises an amino acid sequence having at least 90%, 91%, 92%, 93%, 94%, 95%, 96%, 97%, 98%, 99% or 100% identity with the amino acid sequence of SEQ ID NO: 672.

In some embodiments, the IL-15 comprises an amino acid sequence having at least 90%, 91%, 92%, 93%, 94%, 95%, 96%, 97%, 98%, 99% or 100% identity with the amino acid sequence of SEQ ID NO: 2594.

In some embodiments, the IL-15 comprises an amino acid sequence having at least 90%, 91%, 92%, 93%, 94%, 95%, 96%, 97%, 98%, 99% or 100% identity with the amino acid sequence of SEQ ID NO: 673.

In some embodiments, mbIL-15RA comprises an amino acid sequence having at least 90%, 91%, 92%, 93%, 94%, 95%, 96%, 97%, 98%, 99% or 100% identity with the amino acid sequence of SEQ ID NO: 674.

In some embodiments, the IL-15 comprises an amino acid sequence having at least 90%, 91%, 92%, 93%, 94%, 95%, 96%, 97%, 98%, 99% or 100% identity with the amino acid sequence of SEQ ID NO: 2595.

In some embodiments, mbIL-15RA comprises an amino acid sequence having at least 90%, 91%, 92%, 93%, 94%, 95%, 96%, 97%, 98%, 99% or 100% identity with the amino acid sequence of SEQ ID NO: 675.

In some embodiments, an NK cell or population of NK cells comprising (e.g., expressing) a CAR described herein also comprises (e.g., expresses) an IgE Leader-IL-15-SG3-(SG4)5-SG3-1L15Ra, wherein the IgE Leader-IL-15-5G-3-(SG4)5-SG3-IL15Ra polypeptide comprises or consists of an amino acid sequence having at least 90%, 91%, 92%, 93%, 94%, 95%, 96%, 97%, 98%, 99% or 100% identity with the amino acid sequence of SEQ ID NO: 676.

In some embodiments, an NK cell or population of NK cells comprising (e.g., expressing) a CAR described herein also comprises (e.g., expresses) an IgE leader-IL-15-CD8a Tm+hinge polypeptide, wherein the IgE leader-IL-15-CD8a Tm+hinge polypeptide comprises or consists of an amino acid sequence having at least 90%, 91%, 92%, 93%, 94%, 95%, 96%, 97%, 98%, 99% or 100% identity with the amino acid sequence of SEQ ID NO: 677.

In some embodiments, an NK cell or population of NK cells comprising (e.g., expressing) a CAR described herein also comprises (e.g., expresses) a IL15-(GS)15-IL15Ra (206-267) polypeptide, wherein the IL15-(GS)15-IL15Ra (206-267) polypeptide comprises or consists of an amino acid sequence having at least 90%, 91%, 92%, 93%, 94%, 95%, 96%, 97%, 98%, 99% or 100% identity with the amino acid sequence of SEQ ID NO: 678.

Nucleic acids encoding a CAR and a functional effector protein (e.g., cytokine) described herein which may be used to modify an NK cell or population of NK cells are also provided. In some embodiments, a CAR (e.g., an anti-CD70 CAR) and a functional effector protein (e.g., cytokine (e.g., IL-15 or IL-15/IL-15RA)) are each encoded by a separate vector. In some embodiments, a CAR and a functional effector protein (e.g., a cytokine) are encoded by the same vector. In some embodiments, the CAR and the functional effector protein (e.g., a cytokine) are separated by a 2A sequence (e.g., a T2A sequence or a P2A sequence). In some embodiments, the cytokine comprises soluble or secreted IL-15, membrane bound IL-15 (mbIL-15), a IL-15 receptor alpha (mbIL-15RA), a mbIL-15 with co-expressed IL-15Ra, a fusion of IL-15 and IL-15RA, or a soluble IL-15 with co-expressed IL-15RA. In some embodiments, the functional effector protein is a soluble or secreted IL-15 that complexes with co-expressed IL15RA on the NK cell or population of NK cells. The soluble or secreted IL-15 and the IL15RA coding sequences may be separated by an internal ribosome entry site (IRES) sequence or a P2A sequence. In some embodiments, the IL15 and IL-15RA coding sequences are separated by a P2A linker sequence. In some embodiments, the cytokine is an IL-18. In some embodiments, the cytokine is a membrane bound IL-18 (mbIL-18). In some embodiments, the cytokine is an IL-21. In some embodiments, the cytokine is a membrane bound IL-21 (mbIL-21).

In some embodiments, the IL-18 comprises an amino acid sequence having at least 90%, 91%, 92%, 93%, 94%, 95%, 96%, 97%, 98%, 99% or 100% identity with the amino acid sequence of SEQ ID NO: 2596.

In some embodiments, the IL-21 comprises an amino acid sequence having at least 90%, 91%, 92%, 93%, 94%, 95%, 96%, 97%, 98%, 99% or 100% identity with the amino acid sequence of SEQ ID NO: 2597.

In some embodiments, the functional effector element is a chemokine receptor. Chemokines are a group of proteins that regulate cell trafficking and play roles in the regulation of immune response and homing of immune cells to tumors. Transgenic expression of chemokine receptors CCR2b or CXCR2 in T cells enhances trafficking to CCL2- or CXCL1-secreting solid tumors including melanoma and neuroblastoma (Craddock et al. (2010) J. Immunother. 33(8): 780-8 and Kershaw et al. (2002) Hum. Gene Ther. 13(16): 1971-80). Thus, without wishing to be bound by theory, it is believed that chemokine receptors expressed in CAR-expressing cells (e.g., the NK cells provided herein) may facilitate the cell's recognition of chemokines secreted by tumors, e.g., solid tumors, and improve homing of the CAR-expressing cell to the tumor, facilitate the infiltration of the CAR-expressing cell to the tumor, and enhances anti-tumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (e.g., NK cells) described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In some embodiment, the chemokine receptor molecule to be expressed with a CAR described herein is selected based on the chemokine(s) secreted by the tumor. In some embodiments, the CAR-expressing cell described herein further comprises, e.g., expresses, a CCR4 receptor. In some embodiments, the CAR described herein and the chemokine receptor molecule are on the same vector or are on two different vectors. In embodiments where the CAR described herein and the chemokine receptor molecule are on the same vector, the CAR and the chemokine receptor molecule may each be under control of two different promoters or are under the control of the same promoter.

Activity of immunotherapies in cancer, has been limited in part due to the immunosuppressive solid tumor microenvironment (TME). The overproduction of immunosuppressive cytokines, including TGFbeta, by tumor cells and tumor-infiltrating lymphocytes contributes to an immunosuppressive tumor microenvironment. TGFbeta inhibits immune cell function via a variety of mechanisms. TGFbeta is frequently associated with tumor metastasis and invasion, inhibiting the function of immune cells, and poor prognosis in patients with cancer.

In some embodiments, the CAR-expressing NK cell described herein can further express a functional effector element which senses an immunosuppressive signal and inverts it into a cell activation signal, e.g., an agent which enhances the activity of a CAR-expressing cell. In some embodiments, the functional effector element can be an agent which inhibits an inhibitory molecule. Inhibitory molecules, e.g., PD1, can, in some instances, decrease the ability of a CAR-expressing cell to mount an immune effector response. Examples of inhibitory molecules include but are not limited to B7, CD155, PDL1, and TGFβ. In one instance, the functional effector element comprises a first polypeptide, e.g., a polypeptide that detects, recognizes or binds to an immunosuppressive molecule in the tumor microenvironment, associated with a second polypeptide that provides a positive signal to the cell, e.g., an intracellular signaling domain described herein. In some embodiments, the functional effector comprises a first polypeptide, e.g., PD1, TGFBR, or an antigen binding fragment thereof (e.g., at least a portion of an extracellular domain of any of these), and a second polypeptide which is an intracellular signaling domain described herein (e.g., comprising a costimulatory domain (e.g., DAP12, DAP10, OX40, OX40L, 4-1BB, ICOS, CD27 or CD28, e.g., as described herein) and/or an activation domain (e.g., a DAP12, FCER1G or CD3 zeta signaling domain described herein).

In some embodiments, the functional effector element comprises a first polypeptide of TGFBR or a fragment thereof (e.g., at least a portion of an extracellular domain and transmembrane domain of TGF-beta receptor (TGFBR) (e.g., TGF-beta receptor 1 (TGFBR1, used interchangeably herein with TGFBRI) and/or TGF-beta receptor 2 (TGFBR2, used interchangeably herein with TGFBRII; e.g., amino acid residues 1-166, 1-199, 23-166 or 23-199 of NCBI Reference Sequence: NP_003233 or amino acid residues 1-165, 22-165, 1-198 of SEQ ID NO: 679)), and a second polypeptide of an intracellular signaling domain described herein (e.g., a DAP10 costimulatory domain described herein and/or a CD3 zeta activation domain described herein).

In some embodiments, the functional effector element comprises a TGFBR or fragment thereof which a genetic modification. In some embodiments, the genetic modification converts an inhibitory signal to an activating signal. To allow for the enhanced in vivo ability to overcome tumor microenvironment of NK cells, the cells may be engineered to express a functional effector element such as TGFβ signal converter, a TGFβ decoy receptor (e.g, a TGFBR2 dominant negative receptor (TGFBR1DN) or a TGFBR2 dominant negative receptor (TGFBR2DN)). For example, binding of a TGFBR comprising a genetic modification to a TGFβ ligand in the microenvironment can convert inhibitory signals into activating signals, thereby allowing NK cells to simultaneously resist the immune suppression and achieve enhanced activation leading to superior in vitro and in vivo anti-tumor efficacy. Exemplary TGFBR genetic modifications are described in Burga et al. Clin. Cancer Res. 25(14):4400-12 and WO 2021/010951, both of which are incorporated herein by reference. In some embodiments, the TGFBR or fragment thereof comprising a genetic modification is a TGFβ decoy receptor. In some embodiments, the TGFβ decoy receptor comprises the extracellular domain of a TGFβ receptor (e.g., the extracellular domain of TGFBR1 or TGFBR2) and the transmembrane domain of a TGFβ receptor (e.g., the transmembrane domain of TGFBR1 or TGFBR2). In some embodiments, the TGFβ decoy receptor comprises the extracellular domain of TGFBR2 (with or without TGFBR2's signal peptide) and the transmembrane domain of TFGBR2 (e.g., amino acid residues 1-199 or 23-199 of NCBI Reference Sequence: NP_003233 or amino acid residues 1-198 or 22-198 of SEQ ID NO: 679). In some embodiments, a TGFβ decoy receptor comprises the extracellular domain of a TGFβ receptor (e.g., the extracellular domain of TGFBR1 or TGFBR2 (e.g., amino acid residues 1-166 or 23-166 of NCBI Reference Sequence: NP_003233 or amino acid residues 1-165 or 22-165 of SEQ ID NO: 679)) and a heterologous transmembrane domain (e.g., any of the transmembrane domains provided herein (e.g., a CD28 transmembrane domain)). In some embodiments, the TGFβ decoy receptor is TGFBR2DN (e.g., comprising an amino acid sequence having at least 90%, 91%, 92%, 93%, 94%, 95%, 96%, 97%, 98%, 99% or 100% identity to the amino acid sequence of SEQ ID NO: 679 or 2696). TGFBR2DN can function as a cytokine sink to deplete endogenous TGFβ ligand.

In some embodiments, the functional effector element comprises a first polypeptide of PD1 or a fragment thereof (e.g., at least a portion of an extracellular domain and transmembrane domain of PD1), and a second polypeptide of an intracellular signaling domain described herein (e.g., a DAP10 costimulatory domain described herein and/or a CD3 zeta signaling activation domain described herein). In some embodiments, the CAR-expressing cell described herein comprises a switch costimulatory receptor, e.g., as described in WO 2013/019615, which is incorporated herein by reference. PD1 is an inhibitory member of the CD28 family of receptors that also includes CD28, CTLA-4, ICOS, and BTLA.

In some embodiments, the functional effector element comprises an IL-18 receptor or fragment thereof comprising a genetic modification. IL-18BP, a high affinity IL-18 decoy receptor is frequently upregulated in diverse human and mouse tumors and limits the anti-tumor activity of IL-18. For example, a genetic modification of the IL-18 decoy receptor (i.e., decoy resistant IL-18 or DR-18) can maintain signaling potential but does not transduce inhibitory signals from binding to IL-18BP. This can thereby allow NK cells to simultaneously resist the immune suppression and achieve enhanced activation leading to superior in vitro and in vivo anti-tumor efficacy. Exemplary IL-18 decoy receptor genetic modifications are described in Zhou et al. Nature 583(7817): 609-14, 2020 and are incorporated herein by reference. In some embodiments, the IL-18 receptor or fragment thereof comprising a genetic modification is a decoy resistant IL-18 (DR-18).

In some embodiments, the functional effector element may comprise a TGFBR2DN functional effector element polypeptide comprising or consisting of an amino acid sequence having at least 90%, 91%, 92%, 93%, 94%, 95%, 96%, 97%, 98%, 99% or 100% identity with the amino acid sequence of SEQ ID NO: 679 (with or without the signal peptide noted in Table 7). For example, in some embodiments, the functional effector element may comprise a TGFBR2DN functional effector element polypeptide comprising or consisting of amino acid residues 22-198 of SEQ ID NO: 679.

In some embodiments, the functional effector element may comprise a TGFBR2DN functional effector element polypeptide comprising or consisting of an amino acid sequence having at least 90%, 91%, 92%, 93%, 94%, 95%, 96%, 97%, 98%, 99% or 100% identity with the amino acid sequence of SEQ ID NO: 2696 (with or without the signal peptide noted in Table 7). For example, in some embodiments, the functional effector element may comprise a TGFBR2DN functional effector element polypeptide comprising or consisting of amino acid residues 23-205 of SEQ ID NO: 2696.

In some embodiments, the functional effector element may comprise a PD1 functional effector element polypeptide comprising or consisting of an amino acid sequence having at least 90%, 91%, 92%, 93%, 94%, 95%, 96%, 97%, 98%, 99% or 100% identity with the amino acid sequence of SEQ ID NO: 680.

Table 7 provides exemplary sequences of cytokines, linkers and functional effector elements which can be used in the constructs disclosed herein. In some embodiments, the functional effector elements of any one of SEQ ID NOs: 672, 2594, 674, 2595, 676, 677, 678, and 2696 do not comprise the indicated leader peptide sequence.

TABLE 7
Exemplary Construct Components

| | | |
|---|---|---|
| Exemplary | | |
| Construct | | SEQ ID |
| Components | Amino Acid Sequence | NO: |

LINKER

| | |
|---|---|
| GGGGS | 651 |
| GGGGSGGGGSGGGGS | 652 |
| GGSGGSGGYPYDVPDYAGGGSGGGS | 653 |
| GGSGGSGGGGGSGGGSGGGSGGGS | 654 |
| GGSGGSGGGPEDEPGSGSGGGSGGGS | 655 |
| GGSGGSGGGGGSGGGSGGGSGGGSGSGSGSGSEDGSGSGSGS | 656 |
| GSGSGSGSGSEDEDEDEDGSGSGSGSGS | 657 |
| GGGGSGGGGSGGGGSGGGGS | 658 |
| GSGSGSGSEDGSGSGSGS | 659 |
| GSGSGSGSGSGSGSGSGS | 660 |
| GCGGSGGGGSGGGGS | 661 |
| SGRGGGGSGGGGSGGGGSGGGGSSPA | 662 |
| GGGGSGGGGSGGGGSGGGGSGGGG | 663 |
| SGRGASSGSSGSGSQKKPRYEIRWKVVVISAILALVVLTVISLIILIMLW | 664 |
| GSGMQSPA | |

2A SEQUENCE ELEMENTS

| | | |
|---|---|---|
| T2A | GSGEGRGSLLTCGDVEENPGP | 666 |
| P2A | GSGATNFSLLKQAGDVEENPGP | 667 |
| E2A | GSGQCTNYALLKLAGDVESNPGP | 705 |
| F2A | GSGVKQTLNFDLLKLAGDVESNPGP | 706 |

FUNCTIONAL EFFECTOR ELEMENTS

| | | |
|---|---|---|
| IL-12p40 | CPARSLLLVATLVLLDHLSLARNLPVATPDPGMFPCLHHSQNLLRAVSNM | 668 |
| | LQKARQTLEFYPCTSEEIDHEDITKDKTSTVEACLPLELTKNESCLNSRE | |
| | TSFITNGSCLASRKTSFMMALCLSSIYEDLKMYQVEFKTMNAKLLMDPKR | |
| | QIFLDQNMLAVIDELMQALNFNSETVPQKSSLEEPDFYKTKIKLCILLHA | |
| | FRIRAVTIDRVMSYLNAS | |
| IL-12p35 | CHQQLVISWFSLVFLASPLVAIWELKKDVYVVELDWYPDAPGEMVVLTCD | 669 |
| | TPEEDGITWTLDQSSEVLGSGKTLTIQVKEFGDAGQYTCHKGGEVLSHSL | |
| | LLLHKKEDGIWSTDILKDQKEPKNKTFLRCEAKNYSGRFTCWWLTTISTD | |
| | LTFSVKSSRGSSDPQGVTCGAATLSAERVRGDNKEYEYSVECQEDSACPA | |
| | AEESLPIEVMVDAVHKLKYENYTSSFFIRDIIKPDPPKNLQLKPLKNSRQ | |
| | VEVSWEYPDTWSTPHSYFSLTFCVQVQGKSKREKKDRVFTDKTSATVICR | |
| | KNASISVRAQDRYYSSSWSEWASVPCS | |
| membrane-bound | CPARSLLLVATLVLLDHLSLARNLPVATPDPGMFPCLHHSQNLLRAVSNM | 670 |
| IL-12 polypeptide | LQKARQTLEFYPCTSEEIDHEDITKDKTSTVEACLPLELTKNESCLNSRE | |
| “p40-(GS)15- | TSFITNGSCLASRKTSFMMALCLSSIYEDLKMYQVEFKTMNAKLLMDPKR | |
| IL15Ra(206-267)- | QIFLDQNMLAVIDELMQALNFNSETVPQKSSLEEPDFYKTKIKLCILLHA | |
| P2A-p35” | FRIRAVTIDRVMSYLNASGSGSGSGSGSGSGSGSGSGSGSGSGSGSGSVA | |
| IL12p40, linker, IL- | ISTSTVLLCGLSAVSLLACYLKSRQTPPLASVEMEAMEALPVTWGTSSRD | |
| 15, IL15RA, P2A, | EDLENCSHHL [symbol] MCHQQLVISWFSLVFLAS | |
| IL12p35 | PLVAIWELKKDVYVVELDWYPDAPGEMVVLTCDTPEEDGITWTLDQSSEV | |
| | LGSGKTLTIQVKEFGDAGQYTCHKGGEVLSHSLLLLHKKEDGIWSTDILK | |
| | DQKEPKNKTFLRCEAKNYSGRFTCWWLTTISTDLTFSVKSSRGSSDPQGV | |
| | TCGAATLSAERVRGDNKEYEYSVECQEDSACPAAEESLPIEVMVDAVHKL | |
| | KYENYTSSFFIRDIIKPDPPKNLQLKPLKNSRQVEVSWEYPDTWSTPHSY | |
| | FSLTFCVQVQGKSKREKKDRVFTDKTSATVICRKNASISVRAQDRYYSSS | |
| | WSEWASVPCS | |
| IL-15 | MRISKPHLRSISIQCYLCLLLNSHFLTEAGIHVFILGCFSAGLPKTEANW | 672 |
| leader, pro-peptide, | VNVISDLKKIEDLIQSMHIDATLYTESDVHPSCKVTAMKCFLLELQVISL | |
| mature cytokine | ESGDASIHDTVENLIILANNSLSSNGNVTESGCKECEELEEKNIKEFLQS | |
| | FVHIVQMFINTS | |
| IL-15 | RISKPHLRSISIQCYLCLLLNSHFLTEAGIHVFILGCFSAGLPKTEANWV | 2594 |
| leader, pro-peptide, | NVISDLKKIEDLIQSMHIDATLYTESDVHPSCKVTAMKCFLLELQVISLE | |
| mature cytokine | SGDASIHDTVENLIILANNSLSSNGNVTESGCKECEELEEKNIKEFLQSF | |
| | VHIVQMFINTS | |
| IL-15 | NWVNVISDLKKIEDLIQSMHIDATLYTESDVHPSCKVTAMKCFLLELQVI | 673 |
| | SLESGDASIHDTVENLIILANNSLSSNGNVTESGCKECEELEEKNIKEFL | |
| | QSFVHIVQMFINTS | |
| mbIL-15RA | MAPRRARGCRTLGLPALLLLLLLRPPATRGITCPPPMSVEHADIWVKSYS | 674 |
| leader, | LYSRERYICNSGFKRKAGTSSLTECVLNKATNVAHWTTPSLKCIRDPALV | |
| extracellular, | HQRPAPPSTVTTAGVTPQPESLSPSGKEPAASSPSSNNTAATTAAIVPGS | |
| transmembrane | QLMPSKSPSTGTTEISSHESSHGTPSQTTAKNWELTASASHQPPGVYPQG | |
| domain | HSDTTVAISTSTVLLCGLSAVSLLACYLKSRQTPPLASVEMEAMEALPVT | |
| intracellular domain | WGTSSRDEDLENCSHHL | |
| mbIL-15RA | APRRARGCRTLGLPALLLLLLLRPPATRGITCPPPMSVEHADIWVKSYSL | 2595 |
| leader, | YSRERYICNSGFKRKAGTSSLTECVLNKATNVAHWTTPSLKCIRDPALVH | |
| extracellular, | QRPAPPSTVTTAGVTPQPESLSPSGKEPAASSPSSNNTAATTAAIVPGSQ | |
| transmembrane | LMPSKSPSTGTTEISSHESSHGTPSQTTAKNWELTASASHQPPGVYPQGH | |
| domain | SDTTVAISTSTVLLCGLSAVSLLACYLKSRQTPPLASVEMEAMEALPVTW | |
| intracellular domain | GTSSRDEDLENCSHHL | |
| mbIL-15RA | ITCPPPMSVEHADIWVKSYSLYSRERYICNSGFKRKAGTSSLTECVLNKA | 675 |
| | TNVAHWTTPSLKCIRDPALVHQRPAPPSTVTTAGVTPQPESLSPSGKEPA | |
| | ASSPSSNNTAATTAAIVPGSQLMPSKSPSTGTTEISSHESSHGTPSQTTA | |
| | KNWELTASASHQPPGVYPQGHSDTTVAISTSTVLLCGLSAVSLLACYLKS | |
| | RQTPPLASVEMEAMEALPVTWGTSSRDEDLENCSHHL | |
| IgE Leader-IL-15- | MDWTWILFLVAAATRVHSNWVNVISDLKKIEDLIQSMHIDATLYTESDVH | 676 |
| SG3-(SG4)5-SG3- | PSCKVTAMKCFLLELQVISLESGDASIHDTVENLIILANNSLSSNGNVTE | |
| IL15Ra)” | SGCKECEELEEKNIKEFLQSFVHIVQMFINTSSGGGSGGGGSGGGGSGGG | |
| IgE Leader, IL-15, | GSGGGGSGGGGSGGGITCPPPMSVEHADIWVKSYSLYSRERYICNSGFKR | |
| linker, IL-15RA | KAGTSSLTECVLNKATNVAHWTTPSLKCIRDPALVHQRPAPPSTVTTAGV | |
| | TPQPESLSPSGKEPAASSPSSNNTAATTAAIVPGSQLMPSKSPSTGTTEI | |
| | SSHESSHGTPSQTTAKNWELTASASHQPPGVYPQGHSDTTVAISTSTVLL | |
| | CGLSAVSLLACYLKSRQTPPLASVEMEAMEALPVTWGTSSRDEDLENCSH | |
| | HL | |
| IgE leader-IL-15- | MDWTWILFLVAAATRVHSNWVNVISDLKKIEDLIQSMHIDATLYTESDVH | 677 |
| CD8α Tm + hinge | PSCKVTAMKCFLLELQVISLESGDASIHDTVENLIILANNSLSSNGNVTE | |
| IgE Leader, IL-15, | SGCKECEELEEKNIKEFLQSFVHIVQMFINTSTTTPAPRPPTPAPTIASQ | |
| CD8TM, hinge | PLSLRPEACRPAAGGAVHTRGLDFACDIYIWAPLAGTCGVLLLSLVITLY | |
| | C | |
| IgE leader-IL15- | MDWTWILFLVAAATRVHSNWVNVISDLKKIEDLIQSMHIDATLYTESDVH | 678 |
| (GS)15-IL15RA | PSCKVTAMKCFLLELQVISLESGDASIHDTVENLIILANNSLSSNGNVTE | |
| (206-267)” | SGCKECEELEEKNIKEFLQSFVHIVQMFINTSGSGSGSGSGSGSGSGSGS | |
| | GSGSGSGSGSGSVAISTSTVLLCGLSAVSLLACYLKSRQTPPLASVEMEA | |
| | MEALPVTWGTSSRDEDLENCSHHL | |
| IL-18 | AAEPVEDNCINFVAMKFIDNTLYFIAEDDENLESDYFGKLESKLSVIRNL | 2596 |
| | NDQVLFIDQGNRPLFEDMTDSDCRDNAPRTIFIISMYKDSQPRGMAVTIS | |
| | VKCEKISTLSCENKIISFKEMNPPDNIKDTKSDIIFFQRSVPGHDNKMQF | |
| | ESSSYEGYFLACEKERDLFKLILKKEDELGDRSIMFTVQNED | |
| IL-21 | RSSPGNMERIVICLMVIFLGTLVHKSSSQGQDRHMIRMRQLIDIVDQLKN | 2597 |
| | YVNDLVPEFLPAPEDVETNCEWSAFSCFQKAQLKSANTGNNERIINVSIK | |
| | KLKRKPPSTNAGRRQKHRLTCPSCDSYEKKPPKEFLERFKSLLQKMIHQH | |
| | LSSRTHGSEDS | |
| TGFBR2DN (leader | GRGLLRGLWPLHIVLWTRIASTIPPHVQKSVNNDMIVTDNNGAVKFPQLC | 679 |
| peptide sequence | KFCDVRFSTCDNQKSCMSNCSITSICEKPQEVCVAVWRKNDENITLETVC | |
| underlined) | HDPKLPYHDFILEDAASPKCIMKEKKKPGETFFMCSCSSDECNDNIIFSE | |
| | EYNTSNPDLLLVIFQVTGISLLPPLGVAISVIIIFYCYRVNRQQKLSS | |
| PD1 functional | MQIPQAPWPVVWAVLQLGWRPGWFLDSPDRPWNPPTFSPALLVVTEGDNA | 680 |
| effector element | TFTCSFSNTSESFVLNWYRMSPSNQTDKLAAFPEDRSQPGQDCRFRVTQL | |
| | PNGRDFHMSVVRARRNDSGTYLCGAISLAPKAQIKESLRAELRVTERRAE | |
| | VPTAHPSPSPRPAGQFQTLVV | |
| TGFBR2DN | MGRGLLRGLWPLHIVLWTRIASTIPPHVQKSVNNDMIVTDNNGAVKFPQL | 2696 |
| (TGFBR2 ECD | CKFCDVRFSTCDNQKSCMSNCSITSICEKPQEVCVAVWRKNDENITLETV | |
| fused to CD28 | CHDPKLPYHDFILEDAASPKCIMKEKKKPGETFFMCSCSSDECNDNIIFS | |
| transmembrane | EEYNTSNPDLLLVIFQFWVLVVVGGVLACYSLLVTVAFIIFWVCYRVNRQ | |
| domain) (leader | QKLSS | |
| peptide sequence | | |
| underlined) | | |

Table 8 shows exemplary constructs disclosed herein comprising an anti-CD70 CAR and a functional effector element.

TABLE 8
Exemplary constructs comprising an anti-CD70 CAR and a functional effector element.

| | | Antigen | | | | | | | | | | | |
|---|---|---|---|---|---|---|---|---|---|---|---|---|---|
| | | Recognition | | | | | | | | | | | |
| | Signal | Domain | | | Co- | Activation | | | | | | | |
| ID | Peptide | (Binder) | Hinge | TM | stimulatory 1 | domain 2 | P2A | | | P2A | | P2A | |
| CAT-70-008 | CD27 | CD27 ECD | — | CD27 | CD27 | CD3z | P2A | p40 | — | P2A | p35 | — | — |
| CAT-70-009 | CD27 | CD27 ECD | — | CD27 | CD27 | CD3z | P2A | IL-15 | — | P2A | IL-15Rα | — | — |
| CAT-70-010a | CD27 | CD27 ECD | — | CD27 | CD27 | CD3z | P2A | TGFBRII | DAP12 | — | — | — | — |
| | | | | | | | | ECD | ICD | | | | |
| CAT-70-010b | CD27 | CD27 ECD | — | CD27 | CD27 | CD3z | P2A | PD1 ECD | DAP12 | — | — | — | — |
| | | | | | | | | | ICD | | | | |
| CAT-CD70-120 | CD27 | CD27 ECD | — | CD27 | 4-1BB | CD3z | P2A | IL-15 | — | — | — | — | — |
| CAT-CD70-121 | CD27 | CD27 ECD | — | CD27 | 4-1BB | CD3z | P2A | IL-15Rα | — | P2A | IL-15 | — | — |
| CAT-CD70-128 | CD8a | 1F6 | CD8a | CD8a | 4-1BB | CD3z | P2A | IL-15 | — | — | — | — | — |
| CAT-CD70-129 | CD8a | 1F6 | CD8a | CD8a | 4-1BB | CD3z | P2A | IL-15Rα | — | P2A | IL-15 | — | — |
| CAT-CD70-131 | CD8a | 1F6 | IgG1 | CD28 | CD28 | CD3z | P2A | IL-15 | — | — | — | — | — |
| CAT-CD70-132 | CD8a | 1F6 | IgG1 | CD28 | CD28 | CD3z | P2A | IL-15Rα | — | P2A | IL-15 | — | — |
| CAT-CD70-210 | CD27 | CD27 ECD | — | CD27 | CD27 | CD3z | P2A | IL-15 | — | — | — | — | — |
| CAT-CD70-211 | CD8a | 1F6 | CD8a | CD8a | CD28 | CD3z | P2A | IL-15 | — | — | — | — | — |
| CAT-CD70-212 | CD27 | CD27 ECD | — | CD27 | CD27 | CD3z | P2A | IL-15Rα | — | P2A | IL-15 | — | — |
| CAT-CD70-213 | CD8a | 1F6 | CD8a | CD8a | CD28 | CD3z | P2A | IL-15Rα | — | P2A | IL-15 | — | — |
| CAT-CD70-214 | CD27 | CD27 ECD | — | CD27 | CD27 | CD3z | P2A | mbIL12 | — | — | — | — | — |
| CAT-CD70-215 | CD27 | CD27 ECD | — | CD27 | 4-1BB | CD3z | P2A | mbIL12 | — | — | — | — | — |
| CAT-CD70-216 | CD8a | 1F6 | CD8a | CD8a | CD28 | CD3z | P2A | mbIL12 | — | — | — | — | — |
| CAT-CD70-217 | CD8a | 1F6 | CD8a | CD8a | 4-1BB | CD3z | P2A | mbIL12 | — | — | — | — | — |
| CAT-CD70-218 | CD8a | 1F6 | IgG1 | CD28 | CD28 | CD3z | P2A | mbIL12 | — | — | — | — | — |
| CAT-CD70-219 | CD27 | CD27 ECD | — | CD27 | CD27 | CD3z | P2A | IL18 | — | — | — | — | — |
| CAT-CD70-220 | CD27 | CD27 ECD | — | CD27 | 4-1BB | CD3z | P2A | IL18 | — | — | — | — | — |
| CAT-CD70-221 | CD8a | 1F6 | CD8a | CD8a | CD28 | CD3z | P2A | IL18 | — | — | — | — | — |
| CAT-CD70-222 | CD8a | 1F6 | CD8a | CD8a | 4-1BB | CD3z | P2A | IL18 | — | — | — | — | — |
| CAT-CD70-223 | CD8a | 1F6 | IgG1 | CD28 | CD28 | CD3z | P2A | IL18 | — | — | — | — | — |
| CAT-CD70-224 | CD27 | CD27 ECD | — | CD27 | CD27 | CD3z | P2A | IL21 | — | — | — | — | — |
| CAT-CD70-225 | CD27 | CD27 ECD | — | CD27 | 4-1BB | CD3z | P2A | IL21 | — | — | — | — | — |
| CAT-CD70-226 | CD8a | 1F6 | CD8a | CD8a | CD28 | CD3z | P2A | IL21 | — | — | — | — | — |
| CAT-CD70-227 | CD8a | 1F6 | CD8a | CD8a | 4-1BB | CD3z | P2A | IL21 | — | — | — | — | — |
| CAT-CD70-228 | CD8a | 1F6 | IgG1 | CD28 | CD28 | CD3z | P2A | IL21 | — | — | — | — | — |
| CAT-CD70-239 | CD27 | CD27 ECD | — | CD27 | 4-1BB | CD3z | P2A | p40 | — | P2A | p35 | — | — |
| CAT-CD70-240 | CD8a | 1F6 | CD8a | CD8a | 4-1BB | CD3z | P2A | p40 | — | P2A | p35 | — | — |
| CAT-CD70-241 | CD8a | 1F6 | IgG1 | CD28 | CD28 | CD3z | P2A | p40 | — | P2A | p35 | — | — |
| CAT-CD70-243 | CD8a | 1F6 | CD8a | CD8a | CD28 | CD3z | P2A | p40 | — | P2A | p35 | — | — |
| CAT-CD70-246 | CD27 | CD27 ECD | — | CD27 | CD27 | CD3z | P2A | TGFbR2DN | — | — | — | — | — |
| CAT-CD70-247 | CD27 | CD27 ECD | — | CD27 | 4-1BB | CD3z | P2A | TGFbR2DN | — | — | — | — | — |
| CAT-CD70-248 | CD8a | 1F6 | CD8a | CD8a | CD28 | CD3z | P2A | TGFbR2DN | — | — | — | — | — |
| CAT-CD70-249 | CD8a | 1F6 | CD8a | CD8a | 4-1BB | CD3z | P2A | TGFbR2DN | — | — | — | — | — |
| CAT-CD70-250 | CD8a | 1F6 | IgG1 | CD28 | CD28 | CD3z | P2A | TGFbR2DN | — | — | — | — | — |
| CAT-CD70-251 | CD27 | CD27 ECD | — | CD27 | CD27 | CD3z | P2A | TGFbR2DN | — | P2A | IL-15 | — | — |
| CAT-CD70-252 | CD27 | CD27 ECD | — | CD27 | 4-1BB | CD3z | P2A | TGFbR2DN | — | P2A | IL-15 | — | — |
| CAT-CD70-253 | CD8a | 1F6 | CD8a | CD8a | CD28 | CD3z | P2A | TGFbR2DN | — | P2A | IL-15 | — | — |
| CAT-CD70-254 | CD8a | 1F6 | CD8a | CD8a | 4-1BB | CD3z | P2A | TGFbR2DN | — | P2A | IL-15 | — | — |
| CAT-CD70-255 | CD8a | 1F6 | IgG1 | CD28 | CD28 | CD3z | P2A | TGFbR2DN | — | P2A | IL-15 | — | — |
| CAT-CD70-256 | CD27 | CD27 ECD | — | CD27 | CD27 | CD3z | P2A | TGFbR2DN | — | P2A | IL-15Rα | P2A | IL-15 |
| CAT-CD70-257 | CD27 | CD27 ECD | — | CD27 | 4-1BB | CD3z | P2A | TGFbR2DN | — | P2A | IL-15Rα | P2A | IL-15 |
| CAT-CD70-258 | CD8a | 1F6 | CD8a | CD8a | CD28 | CD3z | P2A | TGFbR2DN | — | P2A | IL-15Rα | P2A | IL-15 |
| CAT-CD70-259 | CD8a | 1F6 | CD8a | CD8a | 4-1BB | CD3z | P2A | TGFbR2DN | — | P2A | IL-15Rα | P2A | IL-15 |
| CAT-CD70-260 | CD8a | 1F6 | IgG1 | CD28 | CD28 | CD3z | P2A | TGFbR2DN | — | P2A | IL-15Rα | P2A | IL-15 |

Table 9 shows exemplary sequences of constructs disclosed herein comprising an anti-CD70 CAR and a functional effector element. In some embodiments, the exemplary sequences of constructs of any one of SEQ ID NOs: 701-704 or 2598-2641 does not comprise the indicated signal peptide(s).

TABLE 9
Exemplary Sequences of constructs comprising an anti-CD70 CAR and a
functional effector element.

| Exemplary CAR | | SEQ |
|---|---|---|
| Name and Domains | Amino Acid Sequence | ID NO: |
| CAT-70-008 | MARPHPWWLCVLGTLVGLSATPAPKSCPERHYWAQGKLCCQMCEPGTFLV | 701 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPCPARSL | |
| domain, P2A, p40, | LLVATLVLLDHLSLARNLPVATPDPGMFPCLHHSQNLLRAVSNMLQKARQ | |
| P2A, p35 | TLEFYPCTSEEIDHEDITKDKTSTVEACLPLELTKNESCLNSRETSFITN | |
| | GSCLASRKTSFMMALCLSSIYEDLKMYQVEFKTMNAKLLMDPKRQIFLDQ | |
| | NMLAVIDELMQALNFNSETVPQKSSLEEPDFYKTKIKLCILLHAFRIRAV | |
| | TIDRVMSYLNASGSGATNFSLLKQAGDVEENPGPCHQQLVISWFSLVFLA | |
| | SPLVAIWELKKDVYVVELDWYPDAPGEMVVLTCDTPEEDGITWTLDQSSE | |
| | VLGSGKTLTIQVKEFGDAGQYTCHKGGEVLSHSLLLLHKKEDGIWSTDIL | |
| | KDQKEPKNKTFLRCEAKNYSGRFTCWWLTTISTDLTFSVKSSRGSSDPQG | |
| | VTCGAATLSAERVRGDNKEYEYSVECQEDSACPAAEESLPIEVMVDAVHK | |
| | LKYENYTSSFFIRDIIKPDPPKNLQLKPLKNSRQVEVSWEYPDTWSTPHS | |
| | YFSLTFCVQVQGKSKREKKDRVFTDKTSATVICRKNASISVRAQDRYYSS | |
| | SWSEWASVPCS | |
| CAT-70-009 | MARPHPWWLCVLGTLVGLSATPAPKSCPERHYWAQGKLCCQMCEPGTFLV | 702 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPRISKPH | |
| domain, P2A, IL-15, | LRSISIQCYLCLLLNSHFLTEAGIHVFILGCFSAGLPKTEANWVNVISDL | |
| P2A, IL-15Rα | KKIEDLIQSMHIDATLYTESDVHPSCKVTAMKCFLLELQVISLESGDASI | |
| | HDTVENLIILANNSLSSNGNVTESGCKECEELEEKNIKEFLQSFVHIVQM | |
| | FINTSGSGATNFSLLKQAGDVEENPGPAPRRARGCRTLGLPALLLLLLLR | |
| | PPATRGITCPPPMSVEHADIWVKSYSLYSRERYICNSGFKRKAGTSSLTE | |
| | CVLNKATNVAHWTTPSLKCIRDPALVHQRPAPPSTVTTAGVTPQPESLSP | |
| | SGKEPAASSPSSNNTAATTAAIVPGSQLMPSKSPSTGTTEISSHESSHGT | |
| | PSQTTAKNWELTASASHQPPGVYPQGHSDTTVAISTSTVLLCGLSAVSLL | |
| | ACYLKSRQTPPLASVEMEAMEALPVTWGTSSRDEDLENCSHHL | |
| CAT-70-010a | MARPHPWWLCVLGTLVGLSATPAPKSCPERHYWAQGKLCCQMCEPGTFLV | 703 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPMGRGLL | |
| domain, P2A, | RGLWPLHIVLWTRIASTIPPHVQKSVNNDMIVTDNNGAVKFPQLCKFCDV | |
| TGFBRII extracellular | RFSTCDNQKSCMSNCSITSICEKPQEVCVAVWRKNDENITLETVCHDPKL | |
| domain, DAP12 | PYHDFILEDAASPKCIMKEKKKPGETFFMCSCSSDECNDNIIFSEEYNTS | |
| signaling domain | NPDLLLVIFQVTGISLLPPLGVAISVIIIFYCYRVNRQQKLSSYFLGRLV | |
| | PRGRGAAEAATRKQRITETESPYQELQGQRSDVYSDLNTQRPYYK | |
| CAT-70-010b | MARPHPWWLCVLGTLVGLSATPAPKSCPERHYWAQGKLCCQMCEPGTFLV | 704 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPMQIPQA | |
| domain, P2A, PD1 | PWPVVWAVLQLGWRPGWFLDSPDRPWNPPTFSPALLVVTEGDNATFTCSF | |
| extracellular domain, | SNTSESFVLNWYRMSPSNQTDKLAAFPEDRSQPGQDCRFRVTQLPNGRDF | |
| DAP12 domain | HMSVVRARRNDSGTYLCGAISLAPKAQIKESLRAELRVTERRAEVPTAHP | |
| | SPSPRPAGQFQTLVVLRPVQAQAQSDCSCSTVSPGVLAGIVMGDLVLTVL | |
| | IALAVYFLGRLVPRGRGAAEAATRKQRITETESPYQELQGQRSDVYSDLN | |
| | TQRPYYK | |
| CAT-CD70-120 | MARPHPWWLCVLGTLVGLSATPAPKSCPERHYWAQGKLCCQMCEPGTFLV | 2598 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPRISKPHLRSISI | |
| domain, P2A, IL-15 | QCYLCLLLNSHFLTEAGIHVFILGCFSAGLPKTEANWVNVISDLKKIEDL | |
| | IQSMHIDATLYTESDVHPSCKVTAMKCFLLELQVISLESGDASIHDTVEN | |
| | LIILANNSLSSNGNVTESGCKECEELEEKNIKEFLQSFVHIVQMFINTS | |
| CAT-CD70-121 | MARPHPWWLCVLGTLVGLSATPAPKSCPERHYWAQGKLCCQMCEPGTFLV | 2599 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPAPRRARGCRTLG | |
| domain, P2A, IL- | LPALLLLLLLRPPATRGITCPPPMSVEHADIWVKSYSLYSRERYICNSGF | |
| 15Rα, P2A, IL-15 | KRKAGTSSLTECVLNKATNVAHWTTPSLKCIRDPALVHQRPAPPSTVTTA | |
| | GVTPQPESLSPSGKEPAASSPSSNNTAATTAAIVPGSQLMPSKSPSTGTT | |
| | EISSHESSHGTPSQTTAKNWELTASASHQPPGVYPQGHSDTTVAISTSTV | |
| | LLCGLSAVSLLACYLKSRQTPPLASVEMEAMEALPVTWGTSSRDEDLENC | |
| | SHHLGSGATNFSLLKQAGDVEENPGPRISKPHLRSISIQCYLCLLLNSHF | |
| | LTEAGIHVFILGCFSAGLPKTEANWVNVISDLKKIEDLIQSMHIDATLYT | |
| | ESDVHPSCKVTAMKCFLLELQVISLESGDASIHDTVENLIILANNSLSSN | |
| | GNVTESGCKECEELEEKNIKEFLQSFVHIVQMFINTS | |
| CAT-CD70-128 | MALPVTALLLPLALLLHAARPQVQLVQSGAEVKKPGASVKVSCKASGYTF | 2600 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, IL-15 | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPRGSGATNFSLLKQAGDVEENPGPRISKPHLRSISIQCYLCLLLNSHF | |
| | LTEAGIHVFILGCFSAGLPKTEANWVNVISDLKKIEDLIQSMHIDATLYT | |
| | ESDVHPSCKVTAMKCFLLELQVISLESGDASIHDTVENLIILANNSLSSN | |
| | GNVTESGCKECEELEEKNIKEFLQSFVHIVQMFINTS | |
| CAT-CD70-129 | MALPVTALLLPLALLLHAARPQVQLVQSGAEVKKPGASVKVSCKASGYTF | 2601 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, IL- | DAPAYQQGQNQLYNELNLGRREEYDVLDKRRGRDPEMGGKPRRKNPQEGL | |
| 15Rα, P2A, IL-15 | YNELQKDKMAEAYSEIGMKGERRRGKGHDGLYQGLSTATKDTYDALHMQA | |
| | LPPRGSGATNFSLLKQAGDVEENPGPAPRRARGCRTLGLPALLLLLLLRP | |
| | PATRGITCPPPMSVEHADIWVKSYSLYSRERYICNSGFKRKAGTSSLTEC | |
| | VLNKATNVAHWTTPSLKCIRDPALVHQRPAPPSTVTTAGVTPQPESLSPS | |
| | GKEPAASSPSSNNTAATTAAIVPGSQLMPSKSPSTGTTEISSHESSHGTP | |
| | SQTTAKNWELTASASHQPPGVYPQGHSDTTVAISTSTVLLCGLSAVSLLA | |
| | CYLKSRQTPPLASVEMEAMEALPVTWGTSSRDEDLENCSHHLGSGATNFS | |
| | LLKQAGDVEENPGPRISKPHLRSISIQCYLCLLLNSHFLTEAGIHVFILG | |
| | CFSAGLPKTEANWVNVISDLKKIEDLIQSMHIDATLYTESDVHPSCKVTA | |
| | MKCFLLELQVISLESGDASIHDTVENLIILANNSLSSNGNVTESGCKECE | |
| | ELEEKNIKEFLQSFVHIVQMFINTS | |
| CAT-CD70-131 | MALPVTALLLPLALLLHAARPQVQLVQSGAEVKKPGASVKVSCKASGYTF | 2602 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTEGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, IL-15 | TLPPSRDELTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGKFW | |
| | VLVVVGGVLACYSLLVTVAFIIFWV [symbol] [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | RISKPHLRSISIQCYLCLLLNSHFLTEAGIHVFILGCFSAGLPKTEANWV | |
| | NVISDLKKIEDLIQSMHIDATLYTESDVHPSCKVTAMKCFLLELQVISLE | |
| | SGDASIHDTVENLIILANNSLSSNGNVTESGCKECEELEEKNIKEFLQSF | |
| | VHIVQMFINTS | |
| CAT-CD70-132 | MALPVTALLLPLALLLHAARPQVQLVQSGAEVKKPGASVKVSCKASGYTF | 2603 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, IL- | TLPPSRDELTKNQVSLTCLVKGFYPSDIAVEWESNGQPENNYKTTPPVLD | |
| 15Rα, P2A, IL-15 | SDGSFFLYSKLTVDKSRWQQGNVFSCSVMHEALHNHYTQKSLSLSPGKFW | |
| | VLVVVGGVLACYSLLVTVAFIIFWV [symbol] [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | APRRARGCRTLGLPALLLLLLLRPPATRGITCPPPMSVEHADIWVKSYSL | |
| | YSRERYICNSGFKRKAGTSSLTECVLNKATNVAHWTTPSLKCIRDPALVH | |
| | QRPAPPSTVTTAGVTPQPESLSPSGKEPAASSPSSNNTAATTAAIVPGSQ | |
| | LMPSKSPSTGTTEISSHESSHGTPSQTTAKNWELTASASHQPPGVYPQGH | |
| | SDTTVAISTSTVLLCGLSAVSLLACYLKSRQTPPLASVEMEAMEALPVTW | |
| | GTSSRDEDLENCSHHLGSGATNFSLLKQAGDVEENPGPRISKPHLRSISI | |
| | QCYLCLLLNSHFLTEAGIHVFILGCFSAGLPKTEANWVNVISDLKKIEDL | |
| | IQSMHIDATLYTESDVHPSCKVTAMKCFLLELQVISLESGDASIHDTVEN | |
| | LIILANNSLSSNGNVTESGCKECEELEEKNIKEFLQSFVHIVQMFINTS | |
| CAT-CD70-210 | MARPHPWWLCVLGTLVGLSATPAPKSCPERHYWAQGKLCCQMCEPGTFLV | 2604 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPRISKPH | |
| domain, P2A, IL-15 | LRSISIQCYLCLLLNSHFLTEAGIHVFILGCFSAGLPKTEANWVNVISDL | |
| | KKIEDLIQSMHIDATLYTESDVHPSCKVTAMKCFLLELQVISLESGDASI | |
| | HDTVENLIILANNSLSSNGNVTESGCKECEELEEKNIKEFLQSFVHIVQM | |
| | FINTS | |
| CAT-CD70-211 | MALPVTALLLPLALLLHAARPQVQLVQSGAEVKKPGASVKVSCKASGYTF | 2605 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, IL-15 | PAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALP | |
| | PRGSGATNFSLLKQAGDVEENPGPRISKPHLRSISIQCYLCLLLNSHFLT | |
| | EAGIHVFILGCFSAGLPKTEANWVNVISDLKKIEDLIQSMHIDATLYTES | |
| | DVHPSCKVTAMKCFLLELQVISLESGDASIHDTVENLIILANNSLSSNGN | |
| | VTESGCKECEELEEKNIKEFLQSFVHIVQMFINTS | |
| CAT-CD70-212 | MARPHPWWLCVLGTLVGLSATPAPKSCPERHYWAQGKLCCQMCEPGTFLV | 2606 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPAPRRAR | |
| domain, P2A, IL- | GCRTLGLPALLLLLLLRPPATRGITCPPPMSVEHADIWVKSYSLYSRERY | |
| 15Rα, P2A, IL-15 | ICNSGFKRKAGTSSLTECVLNKATNVAHWTTPSLKCIRDPALVHQRPAPP | |
| | STVTTAGVTPQPESLSPSGKEPAASSPSSNNTAATTAAIVPGSQLMPSKS | |
| | PSTGTTEISSHESSHGTPSQTTAKNWELTASASHQPPGVYPQGHSDTTVA | |
| | ISTSTVLLCGLSAVSLLACYLKSRQTPPLASVEMEAMEALPVTWGTSSRD | |
| | EDLENCSHHLGSGATNFSLLKQAGDVEENPGPRISKPHLRSISIQCYLCL | |
| | LLNSHFLTEAGIHVFILGCFSAGLPKTEANWVNVISDLKKIEDLIQSMHI | |
| | DATLYTESDVHPSCKVTAMKCFLLELQVISLESGDASIHDTVENLIILAN | |
| | NSLSSNGNVTESGCKECEELEEKNIKEFLQSFVHIVQMFINTS | |
| CAT-CD70-213 | MALPVTALLLPLALLLHAARPQVQLVQSGAEVKKPGASVKVSCKASGYTF | 2607 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, IL- | PAYQQGQNQLYNELNLGRREEYDVLDKRRGRDPEMGGKPRRKNPQEGLYN | |
| 15Rα, P2A, IL-15 | ELQKDKMAEAYSEIGMKGERRRGKGHDGLYQGLSTATKDTYDALHMQALP | |
| | PRGSGATNFSLLKQAGDVEENPGPAPRRARGCRTLGLPALLLLLLLRPPA | |
| | TRGITCPPPMSVEHADIWVKSYSLYSRERYICNSGFKRKAGTSSLTECVL | |
| | NKATNVAHWTTPSLKCIRDPALVHQRPAPPSTVTTAGVTPQPESLSPSGK | |
| | EPAASSPSSNNTAATTAAIVPGSQLMPSKSPSTGTTEISSHESSHGTPSQ | |
| | TTAKNWELTASASHQPPGVYPQGHSDTTVAISTSTVLLCGLSAVSLLACY | |
| | LKSRQTPPLASVEMEAMEALPVTWGTSSRDEDLENCSHHLGSGATNFSLL | |
| | KQAGDVEENPGPRISKPHLRSISIQCYLCLLLNSHFLTEAGIHVFILGCF | |
| | SAGLPKTEANWVNVISDLKKIEDLIQSMHIDATLYTESDVHPSCKVTAMK | |
| | CFLLELQVISLESGDASIHDTVENLIILANNSLSSNGNVTESGCKECEEL | |
| | EEKNIKEFLQSFVHIVQMFINTS | |
| CAT-CD70-214 | MARPHPWWLCVLGTLVGLSATPAPKSCPERHYWAQGKLCCQMCEPGTFLV | 2608 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPMCPARS | |
| domain, P2A, mbIL12 | LLLVATLVLLDHLSLARNLPVATPDPGMFPCLHHSQNLLRAVSNMLQKAR | |
| | QTLEFYPCTSEEIDHEDITKDKTSTVEACLPLELTKNESCLNSRETSFIT | |
| | NGSCLASRKTSFMMALCLSSIYEDLKMYQVEFKTMNAKLLMDPKRQIFLD | |
| | QNMLAVIDELMQALNFNSETVPQKSSLEEPDFYKTKIKLCILLHAFRIRA | |
| | VTIDRVMSYLNASGSGSGSGSGSGSGSGSGSGSGSGSGSGSGSVAISTST | |
| | VLLCGLSAVSLLACYLKSRQTPPLASVEMEAMEALPVTWGTSSRDEDLEN | |
| | CSHHLGSGATNFSLLKQAGDVEENPGPMCHQQLVISWFSLVFLASPLVAI | |
| | WELKKDVYVVELDWYPDAPGEMVVLTCDTPEEDGITWTLDQSSEVLGSGK | |
| | TLTIQVKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGVTCGAA | |
| | TLSAERVRGDNKEYEYSVECQEDSACPAAEESLPIEVMVDAVHKLKYENY | |
| | TSSFFIRDIIKPDPPKNLQLKPLKNSRQVEVSWEYPDTWSTPHSYFSLTF | |
| | CVQVQGKSKREKKDRVFTDKTSATVICRKNASISVRAQDRYYSSSWSEWA | |
| | SVPCS | |
| CAT-CD70-215 | MARPHPWWLCVLGTLVGLSATPAPKSCPERHYWAQGKLCCQMCEPGTFLV | 2609 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPMCPARSLLLVAT | |
| domain, P2A, mbIL12 | LVLLDHLSLARNLPVATPDPGMFPCLHHSQNLLRAVSNMLQKARQTLEFY | |
| | PCTSEEIDHEDITKDKTSTVEACLPLELTKNESCLNSRETSFITNGSCLA | |
| | SRKTSFMMALCLSSIYEDLKMYQVEFKTMNAKLLMDPKRQIFLDQNMLAV | |
| | IDELMQALNFNSETVPQKSSLEEPDFYKTKIKLCILLHAFRIRAVTIDRV | |
| | MSYLNASGSGSGSGSGSGSGSGSGSGSGSGSGSGSGSVAISTSTVLLCGL | |
| | SAVSLLACYLKSRQTPPLASVEMEAMEALPVTWGTSSRDEDLENCSHHLG | |
| | SGATNFSLLKQAGDVEENPGPMCHQQLVISWFSLVFLASPLVAIWELKKD | |
| | VYVVELDWYPDAPGEMVVLTCDTPEEDGITWTLDQSSEVLGSGKTLTIQV | |
| | KEFGDAGQYTCHKGGEVLSHSLLLLHKKEDGIWSTDILKDQKEPKNKTFL | |
| | RCEAKNYSGRFTCWWLTTISTDLTFSVKSSRGSSDPQGVTCGAATLSAER | |
| | VRGDNKEYEYSVECQEDSACPAAEESLPIEVMVDAVHKLKYENYTSSFFI | |
| | RDIIKPDPPKNLQLKPLKNSRQVEVSWEYPDTWSTPHSYFSLTFCVQVQG | |
| | KSKREKKDRVFTDKTSATVICRKNASISVRAQDRYYSSSWSEWASVPCS | |
| CAT-CD70-216 | MALPVTALLLPLALLLHAARPQVQLVQSGAEVKKPGASVKVSCKASGYTF | 2610 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, mbIL12 | PAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALP | |
| | PRGSGATNFSLLKQAGDVEENPGPMCPARSLLLVATLVLLDHLSLARNLP | |
| | VATPDPGMFPCLHHSQNLLRAVSNMLQKARQTLEFYPCTSEEIDHEDITK | |
| | DKTSTVEACLPLELTKNESCLNSRETSFITNGSCLASRKTSFMMALCLSS | |
| | IYEDLKMYQVEFKTMNAKLLMDPKRQIFLDQNMLAVIDELMQALNFNSET | |
| | VPQKSSLEEPDFYKTKIKLCILLHAFRIRAVTIDRVMSYLNASGSGSGSG | |
| | SGSGSGSGSGSGSGSGSGSGSGSVAISTSTVLLCGLSAVSLLACYLKSRQ | |
| | TPPLASVEMEAMEALPVTWGTSSRDEDLENCSHHLGSGATNFSLLKQAGD | |
| | VEENPGPMCHQQLVISWFSLVFLASPLVAIWELKKDVYVVELDWYPDAPG | |
| | EMVVLTCDTPEEDGITWTLDQSSEVLGSGKTLTIQVKEFGDAGQYTCHKG | |
| | GEVLSHSLLLLHKKEDGIWSTDILKDQKEPKNKTFLRCEAKNYSGRFTCW | |
| | WLTTISTDLTFSVKSSRGSSDPQGVTCGAATLSAERVRGDNKEYEYSVEC | |
| | QEDSACPAAEESLPIEVMVDAVHKLKYENYTSSFFIRDIIKPDPPKNLQL | |
| | KPLKNSRQVEVSWEYPDTWSTPHSYFSLTFCVQVQGKSKREKKDRVFTDK | |
| | TSATVICRKNASISVRAQDRYYSSSWSEWASVPCS | |
| CAT-CD70-217 | MALPVTALLLPLALLLHAARPQVQLVQSGAEVKKPGASVKVSCKASGYTF | 2611 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, mbIL12 | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPRGSGATNFSLLKQAGDVEENPGPMCPARSLLLVATLVLLDHLSLARN | |
| | LPVATPDPGMFPCLHHSQNLLRAVSNMLQKARQTLEFYPCTSEEIDHEDI | |
| | TKDKTSTVEACLPLELTKNESCLNSRETSFITNGSCLASRKTSFMMALCL | |
| | SSIYEDLKMYQVEFKTMNAKLLMDPKRQIFLDQNMLAVIDELMQALNFNS | |
| | ETVPQKSSLEEPDFYKTKIKLCILLHAFRIRAVTIDRVMSYLNASGSGSG | |
| | SGSGSGSGSGSGSGSGSGSGSGSGSVAISTSTVLLCGLSAVSLLACYLKS | |
| | RQTPPLASVEMEAMEALPVTWGTSSRDEDLENCSHHLGSGATNFSLLKQA | |
| | GDVEENPGPMCHQQLVISWFSLVFLASPLVAIWELKKDVYVVELDWYPDA | |
| | PGEMVVLTCDTPEEDGITWTLDQSSEVLGSGKTLTIQVKEFGDAGQYTCH | |
| | KGGEVLSHSLLLLHKKEDGIWSTDILKDQKEPKNKTFLRCEAKNYSGRFT | |
| | CWWLTTISTDLTFSVKSSRGSSDPQGVTCGAATLSAERVRGDNKEYEYSV | |
| | ECQEDSACPAAEESLPIEVMVDAVHKLKYENYTSSFFIRDIIKPDPPKNL | |
| | QLKPLKNSRQVEVSWEYPDTWSTPHSYFSLTFCVQVQGKSKREKKDRVFT | |
| | DKTSATVICRKNASISVRAQDRYYSSSWSEWASVPCS | |
| CAT-CD70-218 | MALPVTALLLPLALLLHAARPQVQLVQSGAEVKKPGASVKVSCKASGYTF | 2612 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTEGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, mbIL12 | TLPPSRDELTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGKFW | |
| | VLVVVGGVLACYSLLVTVAFIIFWV [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | MCPARSLLLVATLVLLDHLSLARNLPVATPDPGMFPCLHHSQNLLRAVSN | |
| | MLQKARQTLEFYPCTSEEIDHEDITKDKTSTVEACLPLELTKNESCLNSR | |
| | ETSFITNGSCLASRKTSFMMALCLSSIYEDLKMYQVEFKTMNAKLLMDPK | |
| | RQIFLDQNMLAVIDELMQALNFNSETVPQKSSLEEPDFYKTKIKLCILLH | |
| | AFRIRAVTIDRVMSYLNASGSGSGSGSGSGSGSGSGSGSGSGSGSGSGSV | |
| | AISTSTVLLCGLSAVSLLACYLKSRQTPPLASVEMEAMEALPVTWGTSSR | |
| | DEDLENCSHHLGSGATNFSLLKQAGDVEENPGPMCHQQLVISWFSLVFLA | |
| | SPLVAIWELKKDVYVVELDWYPDAPGEMVVLTCDTPEEDGITWTLDQSSE | |
| | VLGSGKTLTIQVKEFGDAGQYTCHKGGEVLSHSLLLLHKKEDGIWSTDIL | |
| | KDQKEPKNKTFLRCEAKNYSGRFTCWWLTTISTDLTFSVKSSRGSSDPQG | |
| | VTCGAATLSAERVRGDNKEYEYSVECQEDSACPAAEESLPIEVMVDAVHK | |
| | LKYENYTSSFFIRDIIKPDPPKNLQLKPLKNSRQVEVSWEYPDTWSTPHS | |
| | YFSLTFCVQVQGKSKREKKDRVFTDKTSATVICRKNASISVRAQDRYYSS | |
| | SWSEWASVPCS | |
| CAT-CD70-219 | MARPHPWWLCVLGTLVGLSATPAPKSCPERHYWAQGKLCCQMCEPGTFLV | 2613 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPAAEPVE | |
| domain, P2A, IL18 | DNCINFVAMKFIDNTLYFIAEDDENLESDYFGKLESKLSVIRNLNDQVLF | |
| | IDQGNRPLFEDMTDSDCRDNAPRTIFIISMYKDSQPRGMAVTISVKCEKI | |
| | STLSCENKIISFKEMNPPDNIKDTKSDIIFFQRSVPGHDNKMQFESSSYE | |
| | GYFLACEKERDLFKLILKKEDELGDRSIMFTVQNED | |
| CAT-CD70-220 | MARPHPWWLCVLGTLVGLSATPAPKSCPERHYWAQGKLCCQMCEPGTFLV | 2614 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPAAEPVEDNCINF | |
| domain, P2A, IL18 | VAMKFIDNTLYFIAEDDENLESDYFGKLESKLSVIRNLNDQVLFIDQGNR | |
| | PLFEDMTDSDCRDNAPRTIFIISMYKDSQPRGMAVTISVKCEKISTLSCE | |
| | NKIISFKEMNPPDNIKDTKSDIIFFQRSVPGHDNKMQFESSSYEGYFLAC | |
| | EKERDLFKLILKKEDELGDRSIMFTVQNED | |
| CAT-CD70-221 | MALPVTALLLPLALLLHAARPQVQLVQSGAEVKKPGASVKVSCKASGYTF | 2615 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, IL18 | PAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALP | |
| | PRGSGATNFSLLKQAGDVEENPGPAAEPVEDNCINFVAMKFIDNTLYFIA | |
| | EDDENLESDYFGKLESKLSVIRNLNDQVLFIDQGNRPLFEDMTDSDCRDN | |
| | APRTIFIISMYKDSQPRGMAVTISVKCEKISTLSCENKIISFKEMNPPDN | |
| | IKDTKSDIIFFQRSVPGHDNKMQFESSSYEGYFLACEKERDLFKLILKKE | |
| | DELGDRSIMFTVQNED | |
| CAT-CD70-222 | MALPVTALLLPLALLLHAARPQVQLVQSGAEVKKPGASVKVSCKASGYTF | 2616 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, IL18 | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPRGSGATNFSLLKQAGDVEENPGPAAEPVEDNCINFVAMKFIDNTLYF | |
| | IAEDDENLESDYFGKLESKLSVIRNLNDQVLFIDQGNRPLFEDMTDSDCR | |
| | DNAPRTIFIISMYKDSQPRGMAVTISVKCEKISTLSCENKIISFKEMNPP | |
| | DNIKDTKSDIIFFQRSVPGHDNKMQFESSSYEGYFLACEKERDLFKLILK | |
| | KEDELGDRSIMFTVQNED | |
| CAT-CD70-223 | MALPVTALLLPLALLLHAARPQVQLVQSGAEVKKPGASVKVSCKASGYTF | 2617 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, IL18 | TLPPSRDELTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGKFW | |
| | VLVVVGGVLACYSLLVTVAFIIFWV [symbol] [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | AAEPVEDNCINFVAMKFIDNTLYFIAEDDENLESDYFGKLESKLSVIRNL | |
| | NDQVLFIDQGNRPLFEDMTDSDCRDNAPRTIFIISMYKDSQPRGMAVTIS | |
| | VKCEKISTLSCENKIISFKEMNPPDNIKDTKSDIIFFQRSVPGHDNKMQF | |
| | ESSSYEGYFLACEKERDLFKLILKKEDELGDRSIMFTVQNED | |
| CAT-CD70-224 | MARPHPWWLCVLGTLVGLSATPAPKSCPERHYWAQGKLCCQMCEPGTFLV | 2618 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPRSSPGN | |
| domain, P2A, IL21 | MERIVICLMVIFLGTLVHKSSSQGQDRHMIRMRQLIDIVDQLKNYVNDLV | |
| | PEFLPAPEDVETNCEWSAFSCFQKAQLKSANTGNNERIINVSIKKLKRKP | |
| | PSTNAGRRQKHRLTCPSCDSYEKKPPKEFLERFKSLLQKMIHQHLSSRTH | |
| | GSEDS | |
| CAT-CD70-225 | MARPHPWWLCVLGTLVGLSATPAPKSCPERHYWAQGKLCCQMCEPGTFLV | 2619 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPRSSPGNMERIVI | |
| domain, P2A, IL21 | CLMVIFLGTLVHKSSSQGQDRHMIRMRQLIDIVDQLKNYVNDLVPEFLPA | |
| | PEDVETNCEWSAFSCFQKAQLKSANTGNNERIINVSIKKLKRKPPSTNAG | |
| | RRQKHRLTCPSCDSYEKKPPKEFLERFKSLLQKMIHQHLSSRTHGSEDS | |
| CAT-CD70-226 | MALPVTALLLPLALLLHAARPQVQLVQSGAEVKKPGASVKVSCKASGYTF | 2620 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, IL21 | PAYQQGQNQLYNELNLGRREEYDVLDKRRGRDPEMGGKPRRKNPQEGLYN | |
| | ELQKDKMAEAYSEIGMKGERRRGKGHDGLYQGLSTATKDTYDALHMQALP | |
| | PRGSGATNFSLLKQAGDVEENPGPRSSPGNMERIVICLMVIFLGTLVHKS | |
| | SSQGQDRHMIRMRQLIDIVDQLKNYVNDLVPEFLPAPEDVETNCEWSAFS | |
| | CFQKAQLKSANTGNNERIINVSIKKLKRKPPSTNAGRRQKHRLTCPSCDS | |
| | YEKKPPKEFLERFKSLLQKMIHQHLSSRTHGSEDS | |
| CAT-CD70-227 | MALPVTALLLPLALLLHAARPQVQLVQSGAEVKKPGASVKVSCKASGYTF | 2621 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, IL21 | DAPAYQQGQNQLYNELNLGRREEYDVLDKRRGRDPEMGGKPRRKNPQEGL | |
| | YNELQKDKMAEAYSEIGMKGERRRGKGHDGLYQGLSTATKDTYDALHMQA | |
| | LPPRGSGATNFSLLKQAGDVEENPGPRSSPGNMERIVICLMVIFLGTLVH | |
| | KSSSQGQDRHMIRMRQLIDIVDQLKNYVNDLVPEFLPAPEDVETNCEWSA | |
| | FSCFQKAQLKSANTGNNERIINVSIKKLKRKPPSTNAGRRQKHRLTCPSC | |
| | DSYEKKPPKEFLERFKSLLQKMIHQHLSSRTHGSEDS | |
| CAT-CD70-228 | MALPVTALLLPLALLLHAARPQVQLVQSGAEVKKPGASVKVSCKASGYTF | 2622 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain [symbol] | REVPWTEGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, IL21 | TLPPSRDELTKNQVSLTCLVKGFYPSDIAVEWESNGQPENNYKTTPPVLD | |
| | SDGSFFLYSKLTVDKSRWQQGNVFSCSVMHEALHNHYTQKSLSLSPGKFW | |
| | VLVVVGGVLACYSLLVTVAFIIFWV [symbol] [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | RSSPGNMERIVICLMVIFLGTLVHKSSSQGQDRHMIRMRQLIDIVDQLKN | |
| | YVNDLVPEFLPAPEDVETNCEWSAFSCFQKAQLKSANTGNNERIINVSIK | |
| | KLKRKPPSTNAGRRQKHRLTCPSCDSYEKKPPKEFLERFKSLLQKMIHQH | |
| | LSSRTHGSEDS | |
| CAT-CD70-239 | MARPHPWWLCVLGTLVGLSATPAPKSCPERHYWAQGKLCCQMCEPGTFLV | 2623 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPCPARSLLLVATL | |
| domain, P2A, p40, | VLLDHLSLARNLPVATPDPGMFPCLHHSQNLLRAVSNMLQKARQTLEFYP | |
| P2A, p35 | CTSEEIDHEDITKDKTSTVEACLPLELTKNESCLNSRETSFITNGSCLAS | |
| | RKTSFMMALCLSSIYEDLKMYQVEFKTMNAKLLMDPKRQIFLDQNMLAVI | |
| | DELMQALNFNSETVPQKSSLEEPDFYKTKIKLCILLHAFRIRAVTIDRVM | |
| | SYLNASGSGATNFSLLKQAGDVEENPGPCHQQLVISWFSLVFLASPLVAI | |
| | WELKKDVYVVELDWYPDAPGEMVVLTCDTPEEDGITWTLDQSSEVLGSGK | |
| | TLTIQVKEFGDAGQYTCHKGGEVLSHSLLLLHKKEDGIWSTDILKDQKEP | |
| | KNKTFLRCEAKNYSGRFTCWWLTTISTDLTFSVKSSRGSSDPQGVTCGAA | |
| | TLSAERVRGDNKEYEYSVECQEDSACPAAEESLPIEVMVDAVHKLKYENY | |
| | TSSFFIRDIIKPDPPKNLQLKPLKNSRQVEVSWEYPDTWSTPHSYFSLTF | |
| | CVQVQGKSKREKKDRVFTDKTSATVICRKNASISVRAQDRYYSSSWSEWA | |
| | SVPCS | |
| CAT-CD70-240 | MALPVTALLLPLALLLHAARPQVQLVQSGAEVKKPGASVKVSCKASGYTF | 2624 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, p40, | DAPAYQQGQNQLYNELNLGRREEYDVLDKRRGRDPEMGGKPRRKNPQEGL | |
| P2A, p35 | YNELQKDKMAEAYSEIGMKGERRRGKGHDGLYQGLSTATKDTYDALHMQA | |
| | LPPRGSGATNFSLLKQAGDVEENPGPCPARSLLLVATLVLLDHLSLARNL | |
| | PVATPDPGMFPCLHHSQNLLRAVSNMLQKARQTLEFYPCTSEEIDHEDIT | |
| | KDKTSTVEACLPLELTKNESCLNSRETSFITNGSCLASRKTSFMMALCLS | |
| | SIYEDLKMYQVEFKTMNAKLLMDPKRQIFLDQNMLAVIDELMQALNFNSE | |
| | TVPQKSSLEEPDFYKTKIKLCILLHAFRIRAVTIDRVMSYLNASGSGATN | |
| | FSLLKQAGDVEENPGPCHQQLVISWFSLVFLASPLVAIWELKKDVYVVEL | |
| | DWYPDAPGEMVVLTCDTPEEDGITWTLDQSSEVLGSGKTLTIQVKEFGDA | |
| | GQYTCHKGGEVLSHSLLLLHKKEDGIWSTDILKDQKEPKNKTFLRCEAKN | |
| | YSGRFTCWWLTTISTDLTFSVKSSRGSSDPQGVTCGAATLSAERVRGDNK | |
| | EYEYSVECQEDSACPAAEESLPIEVMVDAVHKLKYENYTSSFFIRDIIKP | |
| | DPPKNLQLKPLKNSRQVEVSWEYPDTWSTPHSYFSLTFCVQVQGKSKREK | |
| | KDRVFTDKTSATVICRKNASISVRAQDRYYSSSWSEWASVPCS | |
| CAT-CD70-241 | MALPVTALLLPLALLLHAARPQVQLVQSGAEVKKPGASVKVSCKASGYTF | 2625 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, p40, | TLPPSRDELTKNQVSLTCLVKGFYPSDIAVEWESNGQPENNYKTTPPVLD | |
| P2A, p35 | SDGSFFLYSKLTVDKSRWQQGNVFSCSVMHEALHNHYTQKSLSLSPGKFW | |
| | VLVVVGGVLACYSLLVTVAFIIFWV [symbol] [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | CPARSLLLVATLVLLDHLSLARNLPVATPDPGMFPCLHHSQNLLRAVSNM | |
| | LQKARQTLEFYPCTSEEIDHEDITKDKTSTVEACLPLELTKNESCLNSRE | |
| | TSFITNGSCLASRKTSFMMALCLSSIYEDLKMYQVEFKTMNAKLLMDPKR | |
| | QIFLDQNMLAVIDELMQALNFNSETVPQKSSLEEPDFYKTKIKLCILLHA | |
| | FRIRAVTIDRVMSYLNASGSGATNFSLLKQAGDVEENPGPCHQQLVISWF | |
| | SLVFLASPLVAIWELKKDVYVVELDWYPDAPGEMVVLTCDTPEEDGITWT | |
| | LDQSSEVLGSGKTLTIQVKEFGDAGQYTCHKGGEVLSHSLLLLHKKEDGI | |
| | WSTDILKDQKEPKNKTFLRCEAKNYSGRFTCWWLTTISTDLTFSVKSSRG | |
| | SSDPQGVTCGAATLSAERVRGDNKEYEYSVECQEDSACPAAEESLPIEVM | |
| | VDAVHKLKYENYTSSFFIRDIIKPDPPKNLQLKPLKNSRQVEVSWEYPDT | |
| | WSTPHSYFSLTFCVQVQGKSKREKKDRVFTDKTSATVICRKNASISVRAQ | |
| | DRYYSSSWSEWASVPCS | |
| CAT-CD70-243 | MALPVTALLLPLALLLHAARPQVQLVQSGAEVKKPGASVKVSCKASGYTF | 2626 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, p40, | PAYQQGQNQLYNELNLGRREEYDVLDKRRGRDPEMGGKPRRKNPQEGLYN | |
| P2A, p35 | ELQKDKMAEAYSEIGMKGERRRGKGHDGLYQGLSTATKDTYDALHMQALP | |
| | PRGSGATNFSLLKQAGDVEENPGPCPARSLLLVATLVLLDHLSLARNLPV | |
| | ATPDPGMFPCLHHSQNLLRAVSNMLQKARQTLEFYPCTSEEIDHEDITKD | |
| | KTSTVEACLPLELTKNESCLNSRETSFITNGSCLASRKTSFMMALCLSSI | |
| | YEDLKMYQVEFKTMNAKLLMDPKRQIFLDQNMLAVIDELMQALNFNSETV | |
| | PQKSSLEEPDFYKTKIKLCILLHAFRIRAVTIDRVMSYLNASGSGATNFS | |
| | LLKQAGDVEENPGPCHQQLVISWFSLVFLASPLVAIWELKKDVYVVELDW | |
| | YPDAPGEMVVLTCDTPEEDGITWTLDQSSEVLGSGKTLTIQVKEFGDAGQ | |
| | YTCHKGGEVLSHSLLLLHKKEDGIWSTDILKDQKEPKNKTFLRCEAKNYS | |
| | GRFTCWWLTTISTDLTFSVKSSRGSSDPQGVTCGAATLSAERVRGDNKEY | |
| | EYSVECQEDSACPAAEESLPIEVMVDAVHKLKYENYTSSFFIRDIIKPDP | |
| | PKNLQLKPLKNSRQVEVSWEYPDTWSTPHSYFSLTFCVQVQGKSKREKKD | |
| | RVFTDKTSATVICRKNASISVRAQDRYYSSSWSEWASVPCS | |
| CAT-CD70-246 | MARPHPWWLCVLGTLVGLSATPAPKSCPERHYWAQGKLCCQMCEPGTFLV | 2627 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPGRGLLR | |
| domain, P2A, | GLWPLHIVLWTRIASTIPPHVQKSVNNDMIVTDNNGAVKFPQLCKFCDVR | |
| TGFbR2 DN domain | FSTCDNQKSCMSNCSITSICEKPQEVCVAVWRKNDENITLETVCHDPKLP | |
| | YHDFILEDAASPKCIMKEKKKPGETFFMCSCSSDECNDNIIFSEEYNTSN | |
| | PDLLLVIFQVTGISLLPPLGVAISVIIIFYCYRVNRQQKLSS | |
| CAT-CD70-247 | MARPHPWWLCVLGTLVGLSATPAPKSCPERHYWAQGKLCCQMCEPGTFLV | 2628 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPGRGLLRGLWPLH | |
| domain, P2A, | IVLWTRIASTIPPHVQKSVNNDMIVTDNNGAVKFPQLCKFCDVRFSTCDN | |
| TGFbR2 DN domain | QKSCMSNCSITSICEKPQEVCVAVWRKNDENITLETVCHDPKLPYHDFIL | |
| | EDAASPKCIMKEKKKPGETFFMCSCSSDECNDNIIFSEEYNTSNPDLLLV | |
| | IFQVTGISLLPPLGVAISVIIIFYCYRVNRQQKLSS | |
| CAT-CD70-248 | MALPVTALLLPLALLLHAARPQVQLVQSGAEVKKPGASVKVSCKASGYTF | 2629 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, | PAYQQGQNQLYNELNLGRREEYDVLDKRRGRDPEMGGKPRRKNPQEGLYN | |
| TGFbR2 DN domain | ELQKDKMAEAYSEIGMKGERRRGKGHDGLYQGLSTATKDTYDALHMQALP | |
| | PRGSGATNFSLLKQAGDVEENPGPGRGLLRGLWPLHIVLWTRIASTIPPH | |
| | VQKSVNNDMIVTDNNGAVKFPQLCKFCDVRFSTCDNQKSCMSNCSITSIC | |
| | EKPQEVCVAVWRKNDENITLETVCHDPKLPYHDFILEDAASPKCIMKEKK | |
| | KPGETFFMCSCSSDECNDNIIFSEEYNTSNPDLLLVIFQVTGISLLPPLG | |
| | VAISVIIIFYCYRVNRQQKLSS | |
| CAT-CD70-249 | MALPVTALLLPLALLLHAARPQVQLVQSGAEVKKPGASVKVSCKASGYTF | 2630 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, | DAPAYQQGQNQLYNELNLGRREEYDVLDKRRGRDPEMGGKPRRKNPQEGL | |
| TGFbR2 DN domain | YNELQKDKMAEAYSEIGMKGERRRGKGHDGLYQGLSTATKDTYDALHMQA | |
| | LPPRGSGATNFSLLKQAGDVEENPGPGRGLLRGLWPLHIVLWTRIASTIP | |
| | PHVQKSVNNDMIVTDNNGAVKFPQLCKFCDVRFSTCDNQKSCMSNCSITS | |
| | ICEKPQEVCVAVWRKNDENITLETVCHDPKLPYHDFILEDAASPKCIMKE | |
| | KKKPGETFFMCSCSSDECNDNIIFSEEYNTSNPDLLLVIFQVTGISLLPP | |
| | LGVAISVIIIFYCYRVNRQQKLSS | |
| CAT-CD70-250 | MALPVTALLLPLALLLHAARPQVQLVQSGAEVKKPGASVKVSCKASGYTF | 2631 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTEGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, | TLPPSRDELTKNQVSLTCLVKGFYPSDIAVEWESNGQPENNYKTTPPVLD | |
| TGFbR2 DN domain | SDGSFFLYSKLTVDKSRWQQGNVFSCSVMHEALHNHYTQKSLSLSPGKFW | |
| | VLVVVGGVLACYSLLVTVAFIIFWV [symbol] [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | GRGLLRGLWPLHIVLWTRIASTIPPHVQKSVNNDMIVTDNNGAVKFPQLC | |
| | KFCDVRFSTCDNQKSCMSNCSITSICEKPQEVCVAVWRKNDENITLETVC | |
| | HDPKLPYHDFILEDAASPKCIMKEKKKPGETFFMCSCSSDECNDNIIFSE | |
| | EYNTSNPDLLLVIFQVTGISLLPPLGVAISVIIIFYCYRVNRQQKLSS | |
| CAT-CD70-251 | MARPHPWWLCVLGTLVGLSATPAPKSCPERHYWAQGKLCCQMCEPGTFLV | 2632 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPGRGLLR | |
| domain, P2A, | GLWPLHIVLWTRIASTIPPHVQKSVNNDMIVTDNNGAVKFPQLCKFCDVR | |
| TGFbR2 DN domain, | FSTCDNQKSCMSNCSITSICEKPQEVCVAVWRKNDENITLETVCHDPKLP | |
| P2A, IL-15 | YHDFILEDAASPKCIMKEKKKPGETFFMCSCSSDECNDNIIFSEEYNTSN | |
| | PDLLLVIFQVTGISLLPPLGVAISVIIIFYCYRVNRQQKLSSGSGATNFS | |
| | LLKQAGDVEENPGPRISKPHLRSISIQCYLCLLLNSHFLTEAGIHVFILG | |
| | CFSAGLPKTEANWVNVISDLKKIEDLIQSMHIDATLYTESDVHPSCKVTA | |
| | MKCFLLELQVISLESGDASIHDTVENLIILANNSLSSNGNVTESGCKECE | |
| | ELEEKNIKEFLQSFVHIVQMFINTS | |
| CAT-CD70-252 | MARPHPWWLCVLGTLVGLSATPAPKSCPERHYWAQGKLCCQMCEPGTFLV | 2633 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPGRGLLRGLWPLH | |
| domain, P2A, | IVLWTRIASTIPPHVQKSVNNDMIVTDNNGAVKFPQLCKFCDVRFSTCDN | |
| TGFbR2 DN domain, | QKSCMSNCSITSICEKPQEVCVAVWRKNDENITLETVCHDPKLPYHDFIL | |
| P2A, IL-15 | EDAASPKCIMKEKKKPGETFFMCSCSSDECNDNIIFSEEYNTSNPDLLLV | |
| | IFQVTGISLLPPLGVAISVIIIFYCYRVNRQQKLSSGSGATNFSLLKQAG | |
| | DVEENPGPRISKPHLRSISIQCYLCLLLNSHFLTEAGIHVFILGCFSAGL | |
| | PKTEANWVNVISDLKKIEDLIQSMHIDATLYTESDVHPSCKVTAMKCFLL | |
| | ELQVISLESGDASIHDTVENLIILANNSLSSNGNVTESGCKECEELEEKN | |
| | IKEFLQSFVHIVQMFINTS | |
| CAT-CD70-253 | MALPVTALLLPLALLLHAARPQVQLVQSGAEVKKPGASVKVSCKASGYTF | 2634 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, | PAYQQGQNQLYNELNLGRREEYDVLDKRRGRDPEMGGKPRRKNPQEGLYN | |
| TGFbR2 DN domain, | ELQKDKMAEAYSEIGMKGERRRGKGHDGLYQGLSTATKDTYDALHMQALP | |
| P2A, IL-15 | PRGSGATNFSLLKQAGDVEENPGPGRGLLRGLWPLHIVLWTRIASTIPPH | |
| | VQKSVNNDMIVTDNNGAVKFPQLCKFCDVRFSTCDNQKSCMSNCSITSIC | |
| | EKPQEVCVAVWRKNDENITLETVCHDPKLPYHDFILEDAASPKCIMKEKK | |
| | KPGETFFMCSCSSDECNDNIIFSEEYNTSNPDLLLVIFQVTGISLLPPLG | |
| | VAISVIIIFYCYRVNRQQKLSSGSGATNFSLLKQAGDVEENPGPRISKPH | |
| | LRSISIQCYLCLLLNSHFLTEAGIHVFILGCFSAGLPKTEANWVNVISDL | |
| | KKIEDLIQSMHIDATLYTESDVHPSCKVTAMKCFLLELQVISLESGDASI | |
| | HDTVENLIILANNSLSSNGNVTESGCKECEELEEKNIKEFLQSFVHIVQM | |
| | FINTS | |
| CAT-CD70-254 | MALPVTALLLPLALLLHAARPQVQLVQSGAEVKKPGASVKVSCKASGYTF | 2635 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, | DAPAYQQGQNQLYNELNLGRREEYDVLDKRRGRDPEMGGKPRRKNPQEGL | |
| TGFbR2 DN domain, | YNELQKDKMAEAYSEIGMKGERRRGKGHDGLYQGLSTATKDTYDALHMQA | |
| P2A, IL-15 | LPPRGSGATNFSLLKQAGDVEENPGPGRGLLRGLWPLHIVLWTRIASTIP | |
| | PHVQKSVNNDMIVTDNNGAVKFPQLCKFCDVRFSTCDNQKSCMSNCSITS | |
| | ICEKPQEVCVAVWRKNDENITLETVCHDPKLPYHDFILEDAASPKCIMKE | |
| | KKKPGETFFMCSCSSDECNDNIIFSEEYNTSNPDLLLVIFQVTGISLLPP | |
| | LGVAISVIIIFYCYRVNRQQKLSSGSGATNFSLLKQAGDVEENPGPRISK | |
| | PHLRSISIQCYLCLLLNSHFLTEAGIHVFILGCFSAGLPKTEANWVNVIS | |
| | DLKKIEDLIQSMHIDATLYTESDVHPSCKVTAMKCFLLELQVISLESGDA | |
| | SIHDTVENLIILANNSLSSNGNVTESGCKECEELEEKNIKEFLQSFVHIV | |
| | QMFINTS | |
| CAT-CD70-255 | MALPVTALLLPLALLLHAARPQVQLVQSGAEVKKPGASVKVSCKASGYTF | 2636 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTEGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, | TLPPSRDELTKNQVSLTCLVKGFYPSDIAVEWESNGQPENNYKTTPPVLD | |
| TGFbR2 DN domain, | SDGSFFLYSKLTVDKSRWQQGNVFSCSVMHEALHNHYTQKSLSLSPGKFW | |
| P2A, IL-15 | VLVVVGGVLACYSLLVTVAFIIFWV [symbol] [symbol] | |
| | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | GRGLLRGLWPLHIVLWTRIASTIPPHVQKSVNNDMIVTDNNGAVKFPQLC | |
| | KFCDVRFSTCDNQKSCMSNCSITSICEKPQEVCVAVWRKNDENITLETVC | |
| | HDPKLPYHDFILEDAASPKCIMKEKKKPGETFFMCSCSSDECNDNIIFSE | |
| | EYNTSNPDLLLVIFQVTGISLLPPLGVAISVIIIFYCYRVNRQQKLSSGS | |
| | GATNFSLLKQAGDVEENPGPRISKPHLRSISIQCYLCLLLNSHFLTEAGI | |
| | HVFILGCFSAGLPKTEANWVNVISDLKKIEDLIQSMHIDATLYTESDVHP | |
| | SCKVTAMKCFLLELQVISLESGDASIHDTVENLIILANNSLSSNGNVTES | |
| | GCKECEELEEKNIKEFLQSFVHIVQMFINTS | |
| CAT-CD70-256 | MARPHPWWLCVLGTLVGLSATPAPKSCPERHYWAQGKLCCQMCEPGTFLV | 2637 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRG | |
| [symbol] | RDPEMGGKPRRKNPQEGLYNELQKDKMAEAYSEIGMKGERRRGKGHDGLY | |
| CD3z signaling | QGLSTATKDTYDALHMQALPPRGSGATNFSLLKQAGDVEENPGPGRGLLR | |
| domain, P2A, | GLWPLHIVLWTRIASTIPPHVQKSVNNDMIVTDNNGAVKFPQLCKFCDVR | |
| TGFbR2 DN domain, | FSTCDNQKSCMSNCSITSICEKPQEVCVAVWRKNDENITLETVCHDPKLP | |
| P2A, IL-15Rα, P2A, | YHDFILEDAASPKCIMKEKKKPGETFFMCSCSSDECNDNIIFSEEYNTSN | |
| IL-15 | PDLLLVIFQVTGISLLPPLGVAISVIIIFYCYRVNRQQKLSSGSGATNFS | |
| | LLKQAGDVEENPGPAPRRARGCRTLGLPALLLLLLLRPPATRGITCPPPM | |
| | SVEHADIWVKSYSLYSRERYICNSGFKRKAGTSSLTECVLNKATNVAHWT | |
| | TPSLKCIRDPALVHQRPAPPSTVTTAGVTPQPESLSPSGKEPAASSPSSN | |
| | NTAATTAAIVPGSQLMPSKSPSTGTTEISSHESSHGTPSQTTAKNWELTA | |
| | SASHQPPGVYPQGHSDTTVAISTSTVLLCGLSAVSLLACYLKSRQTPPLA | |
| | SVEMEAMEALPVTWGTSSRDEDLENCSHHLGSGATNFSLLKQAGDVEENP | |
| | GPRISKPHLRSISIQCYLCLLLNSHFLTEAGIHVFILGCFSAGLPKTEAN | |
| | WVNVISDLKKIEDLIQSMHIDATLYTESDVHPSCKVTAMKCFLLELQVIS | |
| | LESGDASIHDTVENLIILANNSLSSNGNVTESGCKECEELEEKNIKEFLQ | |
| | SFVHIVQMFINTS | |
| CAT-CD70-257 | MARPHPWWLCVLGTLVGLSATPAPKSCPERHYWAQGKLCCQMCEPGTFLV | 2638 |
| CD27 signal peptide, | KDCDQHRKAAQCDPCIPGVSFSPDHHTRPHCESCRHCNSGLLVRNCTITA | |
| CD27 extracellular | NAECACRNGWQCRDKECTECDPLPNPSLTARSSQALSPHPQPTHLPYVSE | |
| domain, CD27 | MLEARTAGHMQTLADFRQLPARTLSTHWPPQRSLCSSDFIRILVIFSGMF | |
| transmembrane | LVFTLAGALFL [symbol] [symbol] | |
| domain, [symbol] | [symbol] RVKFSRSADAPAYQQGQNQLYNELNLGRREEYDVLDKRRGRDPEMG | |
| [symbol] | GKPRRKNPQEGLYNELQKDKMAEAYSEIGMKGERRRGKGHDGLYQGLSTA | |
| CD3z signaling | TKDTYDALHMQALPPRGSGATNFSLLKQAGDVEENPGPGRGLLRGLWPLH | |
| domain, P2A, | IVLWTRIASTIPPHVQKSVNNDMIVTDNNGAVKFPQLCKFCDVRFSTCDN | |
| TGFbR2 DN domain, | QKSCMSNCSITSICEKPQEVCVAVWRKNDENITLETVCHDPKLPYHDFIL | |
| P2A, IL-15Rα, P2A, | EDAASPKCIMKEKKKPGETFFMCSCSSDECNDNIIFSEEYNTSNPDLLLV | |
| IL-15 | IFQVTGISLLPPLGVAISVIIIFYCYRVNRQQKLSSGSGATNFSLLKQAG | |
| | DVEENPGPAPRRARGCRTLGLPALLLLLLLRPPATRGITCPPPMSVEHAD | |
| | IWVKSYSLYSRERYICNSGFKRKAGTSSLTECVLNKATNVAHWTTPSLKC | |
| | IRDPALVHQRPAPPSTVTTAGVTPQPESLSPSGKEPAASSPSSNNTAATT | |
| | AAIVPGSQLMPSKSPSTGTTEISSHESSHGTPSQTTAKNWELTASASHQP | |
| | PGVYPQGHSDTTVAISTSTVLLCGLSAVSLLACYLKSRQTPPLASVEMEA | |
| | MEALPVTWGTSSRDEDLENCSHHLGSGATNFSLLKQAGDVEENPGPRISK | |
| | PHLRSISIQCYLCLLLNSHFLTEAGIHVFILGCFSAGLPKTEANWVNVIS | |
| | DLKKIEDLIQSMHIDATLYTESDVHPSCKVTAMKCFLLELQVISLESGDA | |
| | SIHDTVENLIILANNSLSSNGNVTESGCKECEELEEKNIKEFLQSFVHIV | |
| | QMFINTS | |
| CAT-CD70-258 | MALPVTALLLPLALLLHAARPQVQLVQSGAEVKKPGASVKVSCKASGYTF | 2639 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSADA | |
| domain, P2A, | PAYQQGQNQLYNELNLGRREEYDVLDKRRGRDPEMGGKPRRKNPQEGLYN | |
| TGFbR2 DN domain, | ELQKDKMAEAYSEIGMKGERRRGKGHDGLYQGLSTATKDTYDALHMQALP | |
| P2A, IL-15Rα, P2A, | PRGSGATNFSLLKQAGDVEENPGPGRGLLRGLWPLHIVLWTRIASTIPPH | |
| IL-15 | VQKSVNNDMIVTDNNGAVKFPQLCKFCDVRFSTCDNQKSCMSNCSITSIC | |
| | EKPQEVCVAVWRKNDENITLETVCHDPKLPYHDFILEDAASPKCIMKEKK | |
| | KPGETFFMCSCSSDECNDNIIFSEEYNTSNPDLLLVIFQVTGISLLPPLG | |
| | VAISVIIIFYCYRVNRQQKLSSGSGATNFSLLKQAGDVEENPGPAPRRAR | |
| | GCRTLGLPALLLLLLLRPPATRGITCPPPMSVEHADIWVKSYSLYSRERY | |
| | ICNSGFKRKAGTSSLTECVLNKATNVAHWTTPSLKCIRDPALVHQRPAPP | |
| | STVTTAGVTPQPESLSPSGKEPAASSPSSNNTAATTAAIVPGSQLMPSKS | |
| | PSTGTTEISSHESSHGTPSQTTAKNWELTASASHQPPGVYPQGHSDTTVA | |
| | ISTSTVLLCGLSAVSLLACYLKSRQTPPLASVEMEAMEALPVTWGTSSRD | |
| | EDLENCSHHLGSGATNFSLLKQAGDVEENPGPRISKPHLRSISIQCYLCL | |
| | LLNSHFLTEAGIHVFILGCFSAGLPKTEANWVNVISDLKKIEDLIQSMHI | |
| | DATLYTESDVHPSCKVTAMKCFLLELQVISLESGDASIHDTVENLIILAN | |
| | NSLSSNGNVTESGCKECEELEEKNIKEFLQSFVHIVQMFINTS | |
| CAT-CD70-259 | MALPVTALLLPLALLLHAARPQVQLVQSGAEVKKPGASVKVSCKASGYTF | 2640 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| CD8α hinge, CD8α | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTFGQGTKVEIKFVPVFLPAKPTTTPAPRPPTPAPTIASQPLSLRP | |
| [symbol] | EACRPAAGGAVHTRGLDFACDIYIWAPLAGTCGVLLLSLVITLYCNHRN [symbol] | |
| CD3z signaling | [symbol] [symbol] RVKFSRSA | |
| domain, P2A, | DAPAYQQGQNQLYNELNLGRREEYDVLDKRRGRDPEMGGKPRRKNPQEGL | |
| TGFbR2 DN domain, | YNELQKDKMAEAYSEIGMKGERRRGKGHDGLYQGLSTATKDTYDALHMQA | |
| P2A, IL-15Rα, P2A, | LPPRGSGATNFSLLKQAGDVEENPGPGRGLLRGLWPLHIVLWTRIASTIP | |
| IL-15 | PHVQKSVNNDMIVTDNNGAVKFPQLCKFCDVRFSTCDNQKSCMSNCSITS | |
| | ICEKPQEVCVAVWRKNDENITLETVCHDPKLPYHDFILEDAASPKCIMKE | |
| | KKKPGETFFMCSCSSDECNDNIIFSEEYNTSNPDLLLVIFQVTGISLLPP | |
| | LGVAISVIIIFYCYRVNRQQKLSSGSGATNFSLLKQAGDVEENPGPAPRR | |
| | ARGCRTLGLPALLLLLLLRPPATRGITCPPPMSVEHADIWVKSYSLYSRE | |
| | RYICNSGFKRKAGTSSLTECVLNKATNVAHWTTPSLKCIRDPALVHQRPA | |
| | PPSTVTTAGVTPQPESLSPSGKEPAASSPSSNNTAATTAAIVPGSQLMPS | |
| | KSPSTGTTEISSHESSHGTPSQTTAKNWELTASASHQPPGVYPQGHSDTT | |
| | VAISTSTVLLCGLSAVSLLACYLKSRQTPPLASVEMEAMEALPVTWGTSS | |
| | RDEDLENCSHHLGSGATNFSLLKQAGDVEENPGPRISKPHLRSISIQCYL | |
| | CLLLNSHFLTEAGIHVFILGCFSAGLPKTEANWVNVISDLKKIEDLIQSM | |
| | HIDATLYTESDVHPSCKVTAMKCFLLELQVISLESGDASIHDTVENLIIL | |
| | ANNSLSSNGNVTESGCKECEELEEKNIKEFLQSFVHIVQMFINTS | |
| CAT-CD70-260 | MALPVTALLLPLALLLHAARPQVQLVQSGAEVKKPGASVKVSCKASGYTF | 2641 |
| CD8α signal peptide, | TNYGMNWVRQAPGQGLKWMGWINTYTGEPTYADAFKGRVTMTRDTSISTA | |
| CD70 scFv (1F6), | YMELSRLRSDDTAVYYCARDYGDYGMDYWGQGTTVTVSSGGGGSGGGGSG | |
| IgG1 hinge, CD28 | GGGSGDIVMTQSPDSLAVSLGERATINCRASKSVSTSGYSFMHWYQQKPG | |
| transmembrane | QPPKLLIYLASNLESGVPDRFSGSGSGTDFTLTISSLQAEDVAVYYCQHS | |
| domain, [symbol] | REVPWTEGQGTKVEIKEPKSCDKTHTCPPCPAPELLGGPSVFLFPPKPKD | |
| [symbol] | TLMISRTPEVTCVVVDVSHEDPEVKFNWYVDGVEVHNAKTKPREEQYNST | |
| CD3z signaling | YRVVSVLTVLHQDWLNGKEYKCKVSNKALPAPIEKTISKAKGQPREPQVY | |
| domain, P2A, | TLPPSRDELTKNQVSLTCLVKGFYPSDIAVEWESNGQPENNYKTTPPVLD | |
| TGFbR2 DN domain, | SDGSFFLYSKLTVDKSRWQQGNVFSCSVMHEALHNHYTQKSLSLSPGKFW | |
| P2A, IL-15Rα, P2A, | VLVVVGGVLACYSLLVTVAFIIFWV [symbol] [symbol] | |
| IL-15 | [symbol] RVKFSRSADAPAYQQGQNQLYNELNLGRREEYDV | |
| | LDKRRGRDPEMGGKPRRKNPQEGLYNELQKDKMAEAYSEIGMKGERRRGK | |
| | GHDGLYQGLSTATKDTYDALHMQALPPRGSGATNFSLLKQAGDVEENPGP | |
| | GRGLLRGLWPLHIVLWTRIASTIPPHVQKSVNNDMIVTDNNGAVKFPQLC | |
| | KFCDVRFSTCDNQKSCMSNCSITSICEKPQEVCVAVWRKNDENITLETVC | |
| | HDPKLPYHDFILEDAASPKCIMKEKKKPGETFFMCSCSSDECNDNIIFSE | |
| | EYNTSNPDLLLVIFQVTGISLLPPLGVAISVIIIFYCYRVNRQQKLSSGS | |
| | GATNFSLLKQAGDVEENPGPAPRRARGCRTLGLPALLLLLLLRPPATRGI | |
| | TCPPPMSVEHADIWVKSYSLYSRERYICNSGFKRKAGTSSLTECVLNKAT | |
| | NVAHWTTPSLKCIRDPALVHQRPAPPSTVTTAGVTPQPESLSPSGKEPAA | |
| | SSPSSNNTAATTAAIVPGSQLMPSKSPSTGTTEISSHESSHGTPSQTTAK | |
| | NWELTASASHQPPGVYPQGHSDTTVAISTSTVLLCGLSAVSLLACYLKSR | |
| | QTPPLASVEMEAMEALPVTWGTSSRDEDLENCSHHLGSGATNFSLLKQAG | |
| | DVEENPGPRISKPHLRSISIQCYLCLLLNSHFLTEAGIHVFILGCFSAGL | |
| | PKTEANWVNVISDLKKIEDLIQSMHIDATLYTESDVHPSCKVTAMKCFLL | |
| | ELQVISLESGDASIHDTVENLIILANNSLSSNGNVTESGCKECEELEEKN | |
| | IKEFLQSFVHIVQMFINTS | |

### 6. CAR Expression Levels

The present disclosure provides a population of engineered NK cells, wherein a plurality of the engineered NK cells of the population comprise any chimeric stimulatory receptor (CAR) disclosed herein. The present disclosure also provides a composition comprising a population of NK cells, wherein a plurality of the NK cells of the population comprise a non-naturally occurring CAR comprising, consisting essentially of, or consisting of: a) an extracellular domain comprising an antigen recognition domain, b) a transmembrane domain, and c) an intracellular domain (e.g., a CAR described herein). The disclosure also provides a composition comprising a population of NK cells, wherein a plurality of the NK cells of the population comprise a non-naturally occurring CAR comprising, consisting essentially of, or consisting of: a) an antigen recognition domain, b) a hinge domain, c) a transmembrane domain, d) a costimulatory domain and e) an activation domain. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the CAR. In some embodiments, the CAR polypeptide is expressed at a copy number of at least 1, 2, 3, 4, 5, 6, 7, 8, 9, 10, 20, 30, 40, 50, 60, 70, 80, 90 or 100 copies per cell. In some embodiments, the nucleic acid encoding the CAR is integrated into the genome at a copy number of at least 1, 2, 3, 4, 5, 6, 7, 8, 9, 10, 20 or 30 copies per cell.

In some embodiments, the NK cells expressing a CAR are further engineered to express at least one cytokine. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the membrane bound cytokine or a cytokine that is co-expressed with a cytokine receptor. In some embodiments, the membrane bound cytokine or cytokine that is co-expressed with a cytokine receptor is expressed at a copy number of at least 1, 2, 3, 4, 5, 6, 7, 8, 9, 10, 20, 25, 30, 40, 50, 60, 70, 75, 80, 90 or 100 copies of polypeptide per cell. In some embodiments, the nucleic acid encoding the membrane bound cytokine or cytokine that is co-expressed with a cytokine receptor is integrated into the genome at a copy number of at least 1, 2, 3, 4, 5, 6, 7, 8, 9, 10, 20 or 30 copies per cell. In some embodiments, the membrane bound cytokine is IL-21. In some embodiments, the membrane bound cytokine is IL-18. In some embodiments, the membrane bound cytokine is IL-12. In some embodiments, the membrane bound cytokine is IL-15. In some embodiments, IL-21 is co-expressed with IL-21R. In some embodiments, IL-18 is co-expressed with IL-18Ra. In some embodiments, IL-12 is co-expressed with IL-12R131. In some embodiments, IL-15 is co-expressed with IL-15RA.

In some embodiments, the NK cells expressing a CAR are further engineered to express CCR4. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the CCR4. In some embodiments, the CCR4 is expressed at a copy number of at least 1, 2, 3, 4, 5, 6, 7, 8, 9, 10, 20, 25, 30, 40, 50, 60, 70, 75, 80, 90 or 100 copies of polypeptide per cell. In some embodiments, the nucleic acid encoding the CCR4 is integrated into the genome at a copy number of at least 1, 2, 3, 4, 5, 6, 7, 8, 9, 10, 20 or 30 copies per cell.

In some embodiments, the NK cells expressing a CAR are further engineered to express a TGFbeta signal converter. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the TGFbeta signal converter. In some embodiments, the TGFbeta signal converter is expressed at a copy number of at least 1, 2, 3, 4, 5, 6, 7, 8, 9, 10, 20, 25, 30, 40, 50, 60, 70, 75, 80, 90 or 100 copies of polypeptide per cell. In some embodiments, the nucleic acid encoding the TGFbeta signal converter is integrated into the genome at a copy number of at least 1, 2, 3, 4, 5, 6, 7, 8, 9, 10, 20 or 30 copies per cell.

In some embodiments, the ratio of the copy number of CAR: IL15 is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10. In some embodiments, the ratio of the copy number of CAR:mbIL-12 is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10. In some embodiments, the ratio of the copy number of CAR:mbIL-21 is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10. In some embodiments, the ratio of the copy number of CAR:mbIL-18 is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10. In some embodiments, the ratio of the copy number of CAR:TGFbeta signal converter is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10. In some embodiments, the ratio of the copy number of CAR:CCR4 is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10. In some embodiments, the ratio of the copy number of CAR:safety switch protein is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10. In some embodiments, the ratio of the copy number of IL15:IL15Ra is about 1:1, 2:1, 3:1, 4:1, 5:1, 6:1, 7:1, 8:1, 9:1, 10:1, 1:2, 1:3, 1:4, 1:5, 1:6, 1:7, 1:8, 1:9 or 1:10.

### 7. Antigens

In some embodiments, provided herein are cells (e.g., NK cells) expressing an anti-CD70 CAR and a second CAR targeting an antigen that is not CD70.

Among the antigens that may be targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, glycosylated antigens, TnAntigens, auto-/self-antigens, tumor-/cancer-associated antigens, and tumor neoantigens (Linnemann et al. Nat. Med. 21(1):81-5, 2015). In particular aspects, the antigens include BCMA, GPRC5D, CD138, CS1, CD19, CD20, CD22, CD79a, CD79b, CD37, CXCR5, CD70, CD96, CD33, CD123, CLEC12a, ADGRE2 or LILRB2. In particular aspects, the antigens for targeting by two or more antigen recognition domains include, but are not limited to CD70 and CD33 (e.g., for AML), CD70 and CD123 (e.g., for AML), CD70 and CLL1 (e.g., for AML), CD70 and CD96 (e.g., for AML); CD70 and CD19 (e.g., for B cell malignancies); CD70 and CD22 (e.g., for B cell malignancies); CD70 and CD20 (e.g., for B cell malignancies); CD70 and CD79a (e.g., for B cell malignancies); CD70 and CD79b (e.g., for B cell malignancies); CD70 and BCMA (e.g., for multiple myeloma); CD70 and GPRC5D (e.g., for multiple myeloma); CD70 and CD138 (e.g., for multiple myeloma); CD70 and CD96 (e.g., for RCC); CD70 and HAVCR1 (e.g., for RCC); CD70 and EGFR (e.g., for RCC). The sequences for these antigens are known in the art, for example, CD33 (e.g., Accession No. NM_001772.4); CD123 (e.g., Accession No. NC 000023.11); CLL1 (e.g., Accession No. NM_138337.6); CD96 (e.g., Accession No. NM_198196.3); CD96 (e.g., Accession No. NM_198196.3); HAVCR1 (e.g., Accession No. NM_001173393.3); EGFR (e.g., Accession No. NM_005228.5); CD19 (e.g., Accession No. NG 007275.1); CD22 (e.g., Accession No. NM_001771.4); CD20 (e.g., Accession No. NM_152866.3); CD79a (e.g., Accession No. NM_001783.4); CD79b (e.g., Accession No. NM_001039933.3); CD37 (e.g., Accession No. NM_001774.3); CXCR5 (e.g., Accession No. NM_001716.5); BCMA (e.g., Accession No. NM_001192.3); GPRC5D (e.g., Accession No. NM_018654.1); and CD138 (e.g., Accession No. NM_001006946.1).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, MAGE 3, and MAGE 4 (or other MAGE antigens such as those disclosed in PCT Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B 1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES 1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B 1, and abnormally expressed intron sequences such as N-acetylglucosaminyltransferase-V; clonal rearrangements of immunoglobulin genes generating unique idiotypes in myeloma and B cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK, SWISS-PROT, and TREMBL.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (HI, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150, envelope glycoprotein B (gB), gH, gN, and pp150. As would be understood by the skilled person, CMV proteins for use as antigens described herein may be identified in public databases such as GENBANK, SWISS-PROT, and TREMBL.

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B.

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GENBANK, SWISS-PROT, and TREMBL.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al. Infect. Immun. 68(6):3740-3, 2000). The complete genome sequence of a virulent strain of Streptococcus pneumoniae has been sequenced and, as would be understood by the skilled person, S. pneumoniae proteins for use herein may also be identified in other public databases such as GENBANK, SWISS-PROT, and TREMBL. Proteins of particular interest for antigens according to the present disclosure include virulence factors and proteins predicted to be exposed at the surface of the pneumococci (see, e.g., Frolet et al. BMC Microbiol. 10:190, 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A Streptococcus polypeptides (e.g., S. pyogenes M proteins), group B Streptococcus (S. agalactiae) polypeptides, Treponema polypeptides, and Yersinia polypeptides (e.g., Y. pestis Fl and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhino sporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Tricho strongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

### 8. Safety Switch Proteins

Although cellular therapies hold great promise for the treatment of human disease, significant toxicities from the cells themselves or from their transgene products have hampered clinical investigation. In some embodiments described herein, immune effector cells (e.g., NK cells) comprising a CAR described herein that have been infused into a mammalian subject, e.g., a human, can be ablated in order to regulate the effect of such immune effector cells should toxicity arise from their use. In some embodiments, the immune cells of the present disclosure may comprise one or more safety switch proteins (e.g., caspase-9, inducible FAS (iFAS), and inducible caspase-9 (icasp9)) or kill switch genes.

As used herein, the term “safety switch protein,” “suicide protein” or “kill switch protein” refers to an engineered protein designed to prevent potential toxicity or otherwise adverse effects of a cell therapy. In some instances, the safety switch protein expression is conditionally controlled to address safety concerns for transplanted engineered cells that have permanently incorporated the gene encoding the safety switch protein into its genome. This conditional regulation could be variable and might include control through a small molecule-mediated post-translational activation and tissue-specific and/or temporal transcriptional regulation. The safety switch could mediate induction of apoptosis, inhibition of protein synthesis or DNA replication, growth arrest, transcriptional and post-transcriptional genetic regulation and/or antibody-mediated depletion. In some instances, the safety switch protein is activated by an exogenous molecule, e.g., a prodrug, that, when activated, triggers apoptosis and/or cell death of a therapeutic cell.

The term “suicide gene” or “kill switch gene” as used herein is defined as a gene which, upon administration of a prodrug, effects transition of a gene product to a compound which kills its host cell. Examples of suicide gene/prodrug combinations which may be used include, but are not limited to inducible caspase 9 (iCASP9) and rimiducid; RQR8 and rituximab; truncated version of EGFR variant III (EGFRv3) and cetuximab; Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include but are not limited to inducible caspase 9 (or caspase 3 or 7), CD20, CD52, EGFRt, or, thymidine kinase, cytosine deaminase, HER1 and any combination thereof. Further suicide genes known in the art that may be used in the present disclosure include Purine nucleoside phosphorylase (PNP), cytochrome p450 enzymes (CYP), carboxypeptidases (CP), carboxylesterase (CE), nitroreductase (NTR), guanine ribosyltransferase (XGRTP), glycosidase enzymes, methionine-γ-lyase (MET), and thymidine phosphorylase (TP).

### 10. NK Cell Activity

In some embodiments, a population of genetically engineered NK cells as disclosed herein exhibits NK cell functions (e.g., effector functions). In some embodiments, the population is cytotoxic to CD70-expressing cells (e.g., CD70-positive tumor cells). In some embodiments, the population exhibits directed secretion of cytolytic granules or engagement of death domain-containing receptors. In some embodiments, the cytolytic granules comprise perforin and/or granzymes. In some embodiments, a NK cell function is degranulation (e.g., CD107a expression), activation (e.g., CD69 production), cytokine production (e.g., TNFalpha or IFNgamma production), target cell line killing or anti-tumor efficacy in mouse models. Illustrative assays for measuring NK cell cytotoxicity and CD107a (granule release) are provided in Li et al., Cell Stem Cell 23:181-192, 2018. In some embodiments, the population exhibits one or more NK cell effector functions at a level that is least 3-4-fold higher than the functions exhibited by a population of NK cells not expressing the first CAR.

### III. Methods

The NK cells for use in the compositions and methods described herein are derived from human peripheral blood mononuclear cells (PBMCs), mobilized peripheral blood stem cells (PBSCs), unstimulated leukapheresis products, human embryonic stem cells (hESCs), induced pluripotent stem cells (iPSCs), mesenchymal stem cells (MSCs), hematopoietic stem cells (HSCs), bone marrow, CD34⁺ cells, or umbilical cord blood (CB), by methods well known in the art (see, e.g., Lowe et al. (2016) Methods Mol. Biol. 1441: 241-51, incorporated herein by reference). In some embodiments, the NK cells are isolated from peripheral blood, CB, bone marrow, or stem cells. The NK cells may be allogeneic or autologous. For example, in some embodiments, a starting population of NK cells for use in the methods described herein is obtained by isolating mononuclear cells using Ficoll density gradient centrifugation and subsequently depleting cells expressing CD3, CD14, and/or CD19. NK cells in the population can be quantified based on the amount of CD56⁺ or CD3⁻/CD56⁺ cells in the resulting population of cells.

Provided herein is a method of making a population of genetically engineered NK cells, the method comprising: (a) providing a population of NK cells; (b) contacting the population of NK cells with a CD70 inhibitor; and (c) expanding the population of NK cells in vitro. In some embodiments, the CD70 inhibitor comprises a small interfering RNA (siRNA) that targets CD70 mRNA, a short hairpin RNA (shRNA) that targets CD70 mRNA, a nucleic acid encoding a siRNA that targets CD70 mRNA, a nucleic acid encoding an shRNA that targets CD70 mRNA, or a combination of any of the foregoing. In some embodiments, the CD70 inhibitor comprises an RNA-guided endonuclease and a guide RNA (gRNA) targeting a CD70 gene. In some embodiments, the CD70 inhibitor comprises a Protein Expression Blocker (PEBL) or a nucleic acid encoding a PEBL, wherein the PEBL comprises a first antigen recognition domain that specifically binds human CD70 and one or more of a localizing domain, an intracellular retention domain and an endoplasmic reticulum (ER) retention domain. In some embodiments, the CD70 inhibitor is an antagonistic anti-CD70 antibody or an antigen-binding fragment thereof.

In some embodiments, (b) contacting the population of NK cells with a CD70 inhibitor may occur prior to (c) expanding the population of NK cells in vitro. In some embodiments, (b) contacting the population of NK cells with a CD70 inhibitor may occur after (c) expanding the population of NK cells in vitro. In some embodiments, (b) contacting the population of NK cells with a CD70 inhibitor may occur concurrently with (c) expanding the population of NK cells in vitro. In some embodiments, (b) contacting the population of NK cells with a CD70 inhibitor may occur prior to, concurrently and/or after (c) expanding the population of NK cells in vitro.

In some embodiments, step (b) contacting the population of NK cells with a CD70 inhibitor occurs at least about 1 day, about 2 days, about 3 days, about 4 days, about 5 days, about 6 days, about 7 days, about 8 days, about 9 days, about 10 days, about 11 days, 12 days, about 13 days, or about 14 days prior to expanding the population of NK cells in vitro. In some embodiments, the contacting of the population of NK cells with a CD70 inhibitor occurs at least about 1 day, about 2 days, about 3 days, about 4 days, about 5 days, about 6 days, about 7 days, about 8 days, about 9 days, about 10 days, about 11 days, 12 days, about 13 days, or about 14 days after the expanding of the population of NK cells in vitro.

In some embodiments, the population of NK cells is a population of human NK cells. In some embodiments, the population of NK cells exhibits at least about 25% greater cell expansion compared to a population of NK cells that is not contacted with the CD70 inhibitor. In some embodiments, the population of NK cells exhibits at least about 30%, about 40%, about 50%, about 60%, about 70%, about 80%, about 90% or about 100% greater cell expansion compared to a population of NK cells that is expanded under the same conditions but is not contacted with the CD70 inhibitor. In some embodiments, the increased expansion results from an increased level of cell proliferation in culture in the population of NK cells contacted with the CD70 inhibitor. In some embodiments, the increased expansion results from a decreased level of cell death in culture in the population of NK cells contacted with the CD70 inhibitor.

In some embodiments, the method of making a population of genetically engineered M (cells, further comprises (d) contacting the population of NK cells with a polynucleotide (e.g., a transposon) encoding a chimeric antigen receptor (CAR) described herein under conditions sufficient to transfer the polynucleotide across a cell membrane of at least one NK cell in the population of NK cells, wherein the first CAR comprises: (i) an extracellular domain comprising any antigen recognition domain that specifically binds human CD70 described herein; (ii) a transmembrane domain described herein; and (iii) an intracellular domain described herein.

In some embodiments, step (d) is performed prior to step (b). In some embodiments, step (d) is performed concurrently with step (b) (e.g., as a single-step process). In some embodiments, step (d) is performed after step (b). In some embodiments, step (d) is performed after step (c).

In some embodiments, step (d) occurs at least about 1 day, about 2 days, about 3 days, about 4 days, about 5 days, about 6 days, about 7 days, about 8 days, about 9 days, about 10 days, about 11 days, about 12 days, about 13 days, or about 14 days prior to step (b). In some embodiments, step (d) occurs at least about 1 day, about days, about 3 days, about 4 days, about 5 days, about 6 days, about 7 days, about 8 days, about 9 days, about 10 days, about 11 days, about 12 days, about 13 days, or about 14 days after to step (b). In some embodiments, step (d) occurs at least about 1 day, about 2 days, about 3 days, about 4 days, about 5 days, about 6 days, about 7 days, about 8 days, about 9 days, about 10 days, about 11 days, about 12 days, about 13 days, or about 14 days after to step (c).

In some embodiments, the method of the disclosure further comprises expanding the population of NK cells in vitro after step (d). In some embodiments, the cells are expanded at least one time, at least two times, at least three times, at least four times, at least five times, or more. In some embodiments, the cells are expanded from about 1 day to about 7 days, about 8 days to about 14 days, about 15 days to about 21 days, about 22 days to about 28 days or about 29 days to about 42 days. In some embodiments, the cells are expanded from about 10 days to about 14 days. In some embodiments, the cells are expanded for about 14 days.

In some embodiments, step (c) comprises expanding the population of NK cells by about 10-100 fold, about 100-1000 fold, about 1000-2000 fold, about 2000-3000 fold, about 3000-4000 fold, about 4000-5000 fold, about 5000-10000 fold, about 10000-20000 fold, 20000-30000 fold, 30000-40000 fold, 40000-50000 fold, 50000-60000 fold or more in culture. In some embodiments, step (c) comprises expanding the population of NK cells by at least 1,000-fold, 2,000-fold, 3,000-fold, 4,000-fold, 5,000-fold, 10,000-fold, 20,000-fold, 30,000-fold, 40,000-fold, 50,000-fold, 60,000-fold, 70,000-fold, 80,000-fold, or more in culture.

In some embodiments, step (b) and/or step (d) comprises use of a viral vector, electroporation, a transposon/transposase system, a lipid nanoparticle or a charge-altering releasable transporter.

In some embodiments, step (b) and/or step (d) comprises the use of a viral vector, and wherein the viral vector is a lentivirus, a gamma retrovirus, an adeno-associated virus, an adenovirus, or a herpes simplex virus. In some embodiments, step (b) and/or step (d) comprises the use of a transposon/transposase system described herein.

In some embodiments, the method of making a population of genetically engineered NK cells, further comprises (e) contacting the population of NK cells with at least one (e.g., one, two, three, or more) additional polynucleotide encoding an additional exogenous polypeptide described herein (e.g., a functional effector element disclosed herein). In some embodiments, step (e) comprises use of a viral vector, electroporation, a transposon/transposase system, a lipid nanoparticle or a charge-altering releasable transporter.

In some embodiments, a single nucleic acid molecule comprises the first polynucleotide (e.g., a polynucleotide encoding a CAR disclosed herein) and the at least one additional polynucleotide (e.g., a polynucleotide encoding a functional effector element disclosed herein). In some embodiments, a first nucleic acid molecule comprises the first polynucleotide and a second nucleic acid molecule comprises the at least one additional polynucleotide. In some embodiments, the at least one additional polynucleotide encodes both a first additional exogenous polypeptide and a second additional exogenous polypeptide.

In some embodiments, the method of making a population of genetically engineered NK cells, further comprises linking an additional exogenous polypeptide (e.g., a functional effector element disclosed herein) to at least one NK cell of the NK cell population by chemical conjugation or using a sortase enzyme disclosed herein.

In some embodiments, the cells are expanded in expansion medium containing L-glutamine. In some embodiments the cells are expanded in AIM-V medium. In some embodiments, the clone selected for expansion demonstrates the capacity to specifically recognize and lyse CD70 expressing target cells.

NK cells may be activated and expanded by any method known in the art (see, e.g., (Shah et al. PLoS One 8(10):e76781, 2013), e.g., the cells may be cultured in suitable basal culture medium (e.g., X-VIVO15, Lympho ONE, NK MACS EL837, and others) supplemented with IL-2 (e.g., 1,000 U/mL) and one or more agents to stimulate growth (e.g., magnetic beads conjugated with anti-NKp46 and anti-CD2, anti-CD137 antibody, 4-1BBL, IL-7, IL-8, IL-12, IL-15, IL-15 receptor antibody, IL-2, and/or IL-21). The NK cells may be co-cultured with artificial antigen-presenting cells or feeder cells (e.g., HMV-II cells, Lu-130 cells, Lu-134-A cells, TCO-2 cells, K562 cells, HFWT cells, EBV-LCL cells, or HUT78 cells, optionally genetically modified to express one or more stimulatory proteins (e.g., IL-21, IL-15, OX40L and/or 4-1BBL). Alternatively, a solid support having on its surface one or more proteins capably of inducing the activation and/or a proliferative response may be used instead of a feeder cell line.

In some embodiments, the NK cells are expanded in the presence of feeder cells (e.g., APCs). In some embodiments the feeder cells are an immortalized cell line. In some embodiments, the feeder cells are autologous cells. In some embodiments, the feeder cells have been irradiated. For example, the recombinant NK cells may be expanded by stimulation with artificial antigen presenting cells, by stimulation with EBC-LCS cells or with T-cells (e.g., Jurkat cell line, CD4+ T cells). In some embodiments, feeder cells (e.g., aAPCs) are genetically engineered, expressing the desired antigen (e.g., CD70) along with costimulatory molecules, such as 4-1BBL, CD28, mbIL-15 and/or mbIL-21, to select for immune cells (e.g., NK cells) in vitro that are capable of sustained CAR-mediated propagation. This powerful technology allows the manufacture of clinically relevant numbers (up to 10¹⁰) of CAR′ NK cells suitable for human application. As needed, additional stimulation cycles can be undertaken to generate larger numbers of genetically modified NK cells. For example, at least 90% of the propagated NK cells express CAR and can be cryopreserved for infusion. Furthermore, this approach can be harnessed to generate NK cells to diverse tumor types by pairing the specificity of the introduced CAR with expression of the tumor-associated antigen (TAA) recognized by the CAR on the aAPC.

In some embodiments, the cells are expanded in the presence of feeder cells at least one time, at least two times, at least three times, at least four times or at least five times. In some embodiments, the cells are expanded in the presence of feeder cells two times. In some embodiments, the cells are expanded in the presence of feeder cells every 1 day, 2 days, 3 days, 4 days, 5 days, 6 days, 7 days, 8 days, 9 days, 10 days, 11 days, 12 days, 13 days or 14 days. In some embodiments, the cells are expanded in the presence of feeder cells for about 1 day to about 7 days, about 8 days to about 14 days, about 15 days to about 21 days, about 22 days to about 28 days or about 29 days to about 42 days. In some embodiments, the cells are expanded in the presence of feeder cells for about 10 days to about 14 days.

In some embodiments, the cells are expanded in the absence of feeder cells from about 1 day to about 7 days, about 8 days to about 14 days, about 15 days to about 21 days, about 22 days to about 28 days or about 29 days to about 42 days. In some embodiments, the cells are expanded in the absence of feeder cells from about 10 days to about 14 days.

Following genetic modification, the cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the chimeric receptor is expanded ex vivo. In some embodiments, the clone is expanded about 10-100 fold, about 100-1000 fold, about 1000-2000 fold, about 2000-3000 fold, about 3000-4000 fold or about 4000-5000 fold in culture. In some embodiments, the clone is expanded at least 1,000-fold in culture.

### IV. Methods of Modified NK-Cell Cryopreservation

In a further aspect, the genetically modified cells may be cryopreserved. In some embodiments of the present disclosure, the NK cells described herein are modified at a point-of-care site. In some cases, modified NK cells are also referred to as engineered NK cells. In some cases, the point-of-care site is at a hospital or at a facility (e.g., a medical facility) near a subject in need of treatment. The subject undergoes apheresis and peripheral blood mononuclear cells (PBMCs) or a sub population of PBMC can be enriched for example, by elutriation or Ficoll separation. Enriched PBMC or a subpopulation of PBMC can be cryopreserved in any appropriate cryopreservation solution prior to further processing. In one instance, the elutriation process is performed using a buffer solution containing human serum albumin. Immune effector cells, such as NK cells can be isolated by selection methods described herein. In one instance, the selection method for NK cells includes beads specific for CD56 on NK cells. In one case, the beads can be paramagnetic beads. The harvested immune effector cells can be cryopreserved in any appropriate cryopreservation solution prior to modification. The immune effector cells can be thawed up to 24 hours, 36 hours, 48 hours. 72 hours or 96 hours ahead of infusion. The thawed cells can be placed in cell culture buffer, for example in cell culture buffer (e.g., RPMI) supplemented with fetal bovine serum (FBS) or human serum AB or placed in a buffer that includes cytokines such as IL-2 and IL-21, prior to modification. In another aspect, the harvested immune effector cells can be modified immediately without the need for cryopreservation.

In one aspect, the population of genetically modified CAR cells is cryopreserved prior to infusion into a subject. Genetically modified CAR cells that are thawed following cryopreservation maintain their ability to bind to the target antigen. In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98% or at least 99% of the cryopreserved genetically modified CAR cells maintain their ability to bind to the target antigen after thawing.

In one aspect, the population of genetically modified CAR cells is immediately infused into a subject. In another aspect, the population of genetically modified CAR cells is placed in a cytokine bath prior to infusion into a subject. In a further aspect, the population of genetically modified CAR cells is cultured and/or stimulated for no more than 1, 2, 3, 4, 5, 6, 7, 14, 21, 28, 35 42 days, 49, 56, 63 or 70 days. In an embodiment, a stimulation includes the co-culture of the genetically modified CAR T cells with aAPCs to promote the growth of CAR positive T cells. In another aspect, the population of genetically modified CAR cells is stimulated for not more than: 1× stimulation, 2× stimulation, 3× stimulation, 4× stimulation, 5× stimulation, 5× stimulation, 6× stimulation, 7× stimulation, 8× stimulation, 9× stimulation or 10× stimulation. In some instances, the genetically modified cells are not cultured ex vivo in the presence of aAPCs. In some specific instances, the method of the embodiment further comprises enriching the cell population for CAR-expressing immune effector cells (e.g., NK cells) after the transfection and/or culturing step. The enriching can comprise fluorescence-activated cell sorting (FACS) to sort for CAR-expressing cells. The enriching can comprise use of a resin (e.g., magnetic bead) to sort for CAR-expressing cells. In a further aspect, the sorting for CAR-expressing cells comprises use of a CAR-binding antibody. The enriching can also comprise depletion of CD56+ cells. In yet still a further aspect of the embodiment, the method further comprises cryopreserving a sample of the population of genetically modified CAR cells.

In some cases, the modified immune effector cells do not undergo a propagation and activation step. In some cases, the modified immune effector cells do not undergo an incubation or culturing step (e.g., ex vivo propagation). In certain cases, the modified immune effector cells are placed in a buffer that includes IL-2 and IL21 prior to infusion. In other instances, the modified immune effector cells are placed or rested in cell culture buffer, for example in cell culture buffer (e.g., RPMI) supplemented with fetal bovine serum (FBS) prior to infusion. Prior to infusion, the modified immune effector cells can be harvested, washed and formulated in saline buffer in preparation for infusion into the subject.

### V. Methods of Gene Delivery and Cell Modification

One of skill in the art would be well-equipped to construct a vector through standard recombinant techniques (see, for example, Sambrook et al., 2001 (supra) and Ausubel et al., 1996 (supra), both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g., derived from Moloney murine leukemia virus vectors (MoMLV), MSCV, SFFV, MPSV, SNV, etc.), lentiviral vectors (e.g. derived from HIV-1, HIV-2, SIV, BIV, FIV, etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, yeast-based vectors, bovine papilloma virus (BPV)-based vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

### 1. Viral Vectors

Viral vectors encoding an antigen receptor, a cytokine and/or a functional effector element may be provided in certain aspects of the methods of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present disclosure are described below.

An engineered virus vector may comprise long terminal repeats (LTRs), a cargo nucleotide sequence, or a cargo cassette. A viral vector-related “cargo cassette” as used herein refers to a nucleotide sequence comprising a left LTR at the 5′ end and a right LTR at the 3′ end, and a nucleotide sequence positioned between the left and right LTRs. The nucleotide sequence flanked by the LTRs is a nucleotide sequence intended for integration into acceptor DNA. A “cargo nucleotide sequence” refers to a nucleotide sequence (e.g., a nucleotide sequence intended for integration into acceptor DNA), flanked by an LTR at each end, wherein the LTRs are heterologous to the nucleotide sequence. A cargo cassette can be artificially engineered.

In some embodiments of the methods of the disclosure, introducing a nucleic acid sequence and/or a genomic editing construct into an immune cell ex vivo, in vivo, in vitro, or in situ comprises a viral vector. In some embodiments, the viral vector is a non-integrating non-chromosomal vector. Exemplary non-integrating non-chromosomal vectors include, but are not limited to, adeno-associated virus (AAV), adenovirus, and herpes viruses. In some embodiments, the viral vector is an integrating chromosomal vector. Integrating chromosomal vectors include, but are not limited to, adeno-associated vectors (AAV), Lentiviruses, and gamma-retroviruses.

Lentiviral vectors are well known in the art (see, for example, U.S. Pat. Nos. 6,013,516 and 5,994,136).

A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Maetzig et al. Viruses 3(6):677-713, 2011.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

In some embodiments of the methods of the disclosure, introducing a nucleic acid sequence and/or a genomic editing construct into an immune cell ex vivo, in vivo, in vitro, or in situ comprises a combination of vectors. Exemplary, non-limiting vector combinations include: viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro, or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro, or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to PD1, any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

A. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence.

(i) Promoter/Enhancers

The expression constructs provided herein comprise a promoter to drive expression of the antigen receptor. To bring a coding sequence “under the control” of a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the lactamase (penicillinase), lactose and tryptophan (trp-) promoter systems. Furthermore, it is contemplated that the control sequences that direct transcription and/or expression of sequences within non-nuclear organelles such as mitochondria, chloroplasts, and the like, can be employed as well.

The promoters employed may be constitutive, tissue-specific, inducible, and/or useful under the appropriate conditions to direct high-level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e.g., beta actin promoter, GADPH promoter, metallothionein promoter; and concatenated response element promoters, such as cyclic AMP response element promoters (ere), serum response element promoter (sre), phorbol ester promoter (TPA) and response element promoters (tre) near a minimal TATA box. It is also possible to use human growth hormone promoter sequences (e.g., the human growth hormone minimal promoter described at GENBANK, accession no. X05244, nucleotide 283-341) or a mouse mammary tumor promoter (available from the ATCC, Cat. No. ATCC 45007). In certain embodiments, the promoter is EF1, EF1alpha, MND, CMV IE, dectin-1, dectin-2, human CD1 lc, F4/80, SM22, RSV, SV40, Ad MLP, beta-actin, MHC class I or MHC class II promoter, U6 promoter or H1 promoter, however any other promoter that is useful to drive expression of the therapeutic gene is applicable to the practice of the present disclosure.

(ii) Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription functional effector elements.

In certain embodiments, the use of internal ribosome entry sites (IRES) elements are used to create multigene, or polycistronic, messages. IRES elements can be linked to heterologous open reading frames. Multiple open reading frames can be transcribed together, each separated by an IRES, creating polycistronic messages.

Additionally, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A) or a P2A (e.g., porcine teschovirus-1 2A).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

B. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker (e.g., genes that confer resistance to neomycin, puromycin, hygromycin, DHFR, GPT, zeocin and histidinol). Other types of markers including screenable markers such as GFP are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### 2. Other Methods of Nucleic Acid Delivery

In addition to viral delivery of the nucleic acids encoding the antigen receptor, the following are additional methods of recombinant gene delivery to a given immune cell (e.g., a NK cell) and are thus considered in the present disclosure. Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

A. Transposition Based Methods of Modification

Generally, the gene transfer system can include a transposon or a viral integration system.

In some embodiments, the gene transfer system comprises a transposon system. DNA transposons can translocate via a non-replicative “cut-and-paste” mechanism. This mechanism requires recognition of the two inverse terminal repeats (ITRs) by a catalytic enzyme, i.e., transposase, which can cleave its target and consequently release the DNA transposon from its donor template. Upon excision, the DNA transposons may subsequently integrate into the acceptor DNA that is cleaved by the same transposase. In some of their natural configurations, DNA transposons are flanked by two ITRs and may contain a gene encoding a transposase that catalyzes transposition.

Transposon systems offer many advantages for nucleic acid integration, e.g., as compared to viral vectors. For example, transposons can carry larger cargos, which can be advantageous for delivering one or more of the CARs, functional effector elements, and/or cytokines disclosed herein, to an immune cell (e.g., an NK cell). Further, transposons may comprise, for example, CRISPR tools (e.g., along with cargo), and thereby allow multiplex engineering of a cell.

A transposon system comprises (i) a plasmid backbone with inverse terminal repeats (ITRs) and (ii) a transposase enzyme that recognizes the ITRs. The term “inverse terminal repeats,” “inverted terminal repeats”, or “ITRs”, as used interchangeably herein, refers to short sequence repeats flanking the transposase gene in a natural transposon, or flanking a cargo polynucleotide sequence in an artificially engineered transposon. Two inverted terminal repeats are generally required for the mobilization of the transposon in the presence of a corresponding transposase. Inverted repeats as described herein may contain one or more direct repeat (DR) sequences. These DR sequences usually are embedded in the terminal inverted repeats (ITRs) of the elements. The compositions and methods of the present disclosure comprise, in various embodiments, one or more artificially engineered transposons. An engineered transposon may comprise ITRs, a cargo nucleotide sequence, or a cargo cassette. A transposon-related “cargo cassette” as used herein refers to a nucleotide sequence comprising a left ITR at the 5′ end and a right ITR at the 3′ end, and a nucleotide sequence positioned between the left and right ITRs. The nucleotide sequence flanked by the ITRs is a nucleotide sequence intended for integration into acceptor DNA. The cargo cassette can, in some embodiments, be comprised in a vector, such as plasmid. A “cargo nucleotide sequence” refers to a nucleotide sequence (e.g., a nucleotide sequence intended for integration into acceptor DNA), flanked by an ITR at each end, wherein the ITRs are heterologous to the nucleotide sequence. A cargo cassette can be artificially engineered.

Transposons and Transposase

Exemplary transposon systems for use as described in the disclosure include, but are not limited to, piggyBac, hyperactive piggyBac, Sleeping Beauty (SB), hyperactive Sleeping Beauty (SB100×), SB11, SB110, Tn7, TcBuster, hyperactive TcBuster, Frog Prince, IS5, Tn10, Tn903, SPIN, hAT, Hermes, Hobo, AeBuster1, AeBuster2, AeBuster3, BtBuster1, BtBuster2, CfBuster1, CfBuster2, Tol2, mini-Tol2, Tc3, Mos1, MuA, Himar I, Helitron and engineered versions of transposase family enzymes (Zhang et al., PLoS Genet. 5:e1000689, 2009; Wilson et al., J. Microbiol. Methods 71:332-5, 2007; the entire contents of which are incorporated by reference herein). Exemplary transposons also include the transposons described in Arensburger et al. (Genetics 188(1):45-57, 2011; the entire contents of which are incorporated by reference herein), or a SPACE INVADERS (SPIN) transposon (see, e.g., Pace et al., Proc. Natl. Acad. Sci. U.S.A. 105(44):17023-17028, 2008; the entire contents of which are incorporated by reference herein). In some embodiments, the gene transfer system can be delivered to the cell encoded in DNA, encoded in mRNA, as a protein, or as a nucleoprotein complex. Alternatively, the gene transfer system can be integrated into the genome of a host cell using, for example, a retro-transposon, random plasmid integration, recombinase-mediated integration, homologous recombination mediated integration, or non-homologous end joining mediated integration. More examples of transposition systems that can be used with certain embodiments of the compositions and methods provided herein include Staphylococcus aureus Tn552 (Colegio et al., J. Bacteriol. 183:2384-8, 2001; Kirby et al., Mol. Microbiol. 43:173-86, 2002), Tyl (Devine & Boeke, Nucleic Acids Res. 22:3765-72, 1994 and International Publication WO 95/23875), Transposon Tn7 (Craig, Science 271:1512, 1996; Craig, Review in: Curr. Top. Microbiol. Immunol. 204:27-48, 1996), Tn/O and IS10 (Kleckner et al., Curr. Top. Microbiol. Immunol. 204:49-82, 1996), Mariner transposase (Lampe et al., EMBO J. 15:5470-9, 1996), Tel (Plasterk, Curr. Topics Microbiol. Immunol. 204:125-43, 1996), P Element (Gloor, Methods Mol. Biol. 260:97-114, 2004), Tn3 (Ichikawa & Ohtsubo, J. Biol. Chem. 265:18829-32, 1990), bacterial insertion sequences (Ohtsubo & Sekine, Curr. Top. Microbiol. Immunol. 204:1-26, 1996), retroviruses (Brown et al., Proc. Natl. Acad. Sci. U.S.A. 86:2525-9, 1989), and retrotransposon of yeast (Boeke & Corces, Ann. Rev. Microbiol. 43:403-34, 1989). The entire contents of each of the foregoing references are incorporated by reference herein.

TcBuster

In some embodiments of the present disclosure, the transposon system is a TcBuster family transposon system. Exemplary TcBuster family transposons of the disclosure include, but are not limited to, the following transposons (wherein the corresponding accession numbers for the appropriate database are shown in parenthesis): (GENBANK database, sequences available on the World Wide Web at ncbi.nlm.nih.gov): Ac-like (AAC46515), Ac (CAA29005), AeBuster1 (ABF20543), AeBuster2 (ABF20544), AmBuster1 (EFB22616), AmBuster2 (EFB25016), AmBuster3 (EFB20710), AmBuster4 (EFB22020), BtBuster1 (ABF22695), BtBuster2 (ABF22700), BtBuster3 (ABF22697), CfBuster1 (ABF22696), CfBuster2 (ABF22701), CfBuster3 (XP_854762), CfBuster4 (XP_545451), CsBuster (ABF20548), Daysleeper (CAB68118), DrBuster1 (ABF20549), DrBuster2 (ABF20550), EcBusterl (XP_001504971), EcBuster3 (XP_001503499), EcBuster4 (XP_001504928), Hermes (AAC37217), hermit (LCU22467), Herves (AAS21248), hobo (A39652), Homer (AAD03082), hopper-we (AAL93203), HsBuster1 (AAF18454), HsBuster2 (ABF22698), HsBuster3 (NP_071373), HsBuster4 (AAS01734), IpTip100 (BAA36225), MamBuster2 (XP_001108973), MamBuster3 (XP_001084430), MamBuster3 (XP_001084430), MamBuster4 (XP_001101327), MmBuster2 (AAF18453), PtBuster2 (ABF22699), PtBuster3 (XP_001142453), PtBuster4 (XP_527300), Restless (CAA93759), RnBuster2 (NP_001102151), SpBuster1 (ABF20546), SpBuster2 (ABF20547), SsBuster4 (XP_001929194), Tam3 (CAA38906), TcBuster (ABF20545), Tol2 (BAA87039), tramp (CAA76545), and XtBuster (ABF20551); (ENSEMBL database, sequences available on the World Wide Web at ensembl.org): PtBuster1 (ENSPTRG00000003364): (REPBASE database, sequences available on the World Wide Web at girinst.org): Ac-like2 (hAT-7_DR), Ac-like1 (hAT-6_DR), hAT-5_DR (hAT-5_DR), MlBuster1 (hAT-4_ML), Myotis-hAT1 (Myotis-hAT1), SPIN_Et (SPIN_Et), SPIN_Ml (SPIN_Ml), and SPIN-Og (SPIN-Og), (TEFam database, sequences available on the World Wide Web at tefam.biochem.vt.edu): AeHermes1 (TF0013337), AeBuster3 (TF001186), AeBuster4 (TF001187), AeBuster5 (TF001188), AeBuster7 (TF001336), AeHermes2 (TF0013338), AeTip100-2 (TF000910), Cx-Kink2 (TF001637), Cx-Kink3 (TF001638), Cx-Kink4 (TF001639), Cx-Kink5 (TF001640), Cx-Kink7 (TF001636), and Cx-Kink8 (TF001635).

Compositions and methods of the disclosure may comprise a TcBuster transposase and/or a TcBuster hyperactive transposase. In some embodiments, compositions and methods of the disclosure comprise a TcBuster transposase, a TcBuster transposon, or a TcBuster transposase and TcBuster transposon. In some embodiments, compositions and methods of the disclosure comprise a hyperactive TcBuster transposase, a TcBuster transposon, or a hyperactive TcBuster transposase and TcBuster transposon. In some embodiments, a hyperactive TcBuster transposase demonstrates an increased excision and/or increased insertion frequency when compared to an excision and/or insertion frequency of a wild type TcBuster transposase.

In some embodiments, a hyperactive TcBuster transposase demonstrates an increased transposition frequency when compared to a transposition frequency of a wild type TcBuster transposase. In some embodiments, a TcBuster transposase may comprise any of the mutations disclosed in WO 2019/246486, which is incorporated herein by reference in its entirety.

In some embodiments of the compositions and methods of the disclosure, a wild type TcBuster transposase comprises or consists of the amino acid sequence of GENBANK Accession No. ABF20545 and SEQ ID NO: 681.

In some embodiments of the compositions and methods of the disclosure, a TcBuster transposase comprises or consists of an amino acid sequence having at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% identity, or any percentage identity in between the foregoing values, or 100% identity, to a wild type TcBuster transposase comprising or consisting of the amino acid sequence of GENBANK Accession No. ABF20545 (SEQ ID NO: 681).

In some embodiments of the compositions and methods of the disclosure, a wild type TcBuster transposase is encoded by a nucleic acid sequence comprising or consisting of the nucleic acid sequence of SEQ ID NO: 682.

In some embodiments of the compositions and methods of the disclosure, a TcBuster Transposase is encoded by a nucleic acid sequence comprising or consisting of a sequence having at least 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identity, or any percentage identity in between the foregoing values, or 100% identity, to a wild type TcBuster transposase encoded by a nucleic acid sequence comprising or consisting of GENBANK Accession No. DQ481197 and SEQ ID NO: 682.

In some embodiments, a recombinant cell, e.g., NK cell produced by transposition-based methods may comprise sequences flanking the nucleotide sequence incorporated into the cell's genome by transposition. Illustrative examples of such flanking sequences (also known as excision footprints) are provided in Woodard et al., (2012) PLoS ONE 7(11): e42666.

Mutant TcBuster Transposase

In some embodiments of the disclosure, the transposase is a mutant TcBuster transposase. Typically, a wild-type TcBuster transposase can be regarded as comprising, from N terminus to C terminus, a ZnF-BED domain (amino acids 76-98), a DNA Binding and Oligomerization domain (amino acids 112-213), a first Catalytic domain (amino acids 213-312), an Insertion domain (amino acids 312-543), and a second Catalytic domain (amino acids 583-620), as well as at least four inter-domain regions in between these annotated domains. Unless indicated otherwise, numerical references to amino acids of a TcBuster transposase, as used herein, are all in accordance to SEQ ID NO: 681. A mutant TcBuster transposase of the disclosure comprises one or more amino acid substitutions in any one of these domains, or any combination thereof. In some embodiments, a mutant TcBuster transposase comprises one or more amino acid substitutions in a ZnF-BED domain, a DNA Binding and Oligomerization domain, a first Catalytic domain, an insertion domain, or a combination thereof. In some embodiments, a mutant TcBuster transposase comprises one or more amino acid substitutions in at least one of the two catalytic domains.

In some embodiments, a mutant TcBuster transposase comprises one or more amino acid substitutions in comparison to a wild-type TcBuster transposase (SEQ ID NO: 681). In some embodiments, the mutant TcBuster transposase comprises an amino acid sequence having at least 70% sequence identity to the full-length sequence of a wild-type TcBuster transposase (SEQ ID NO: 681). In some embodiments, the mutant TcBuster transposase comprises an amino acid sequence having at least 50%, at least 60%, at least 70%, at least 80%, at least 85%, at least 90%, at least 91%, at least 92%, at least 93%, at least 94%, at least 95%, at least 96%, at least 97%, at least 98%, or at least 99% sequence identity to full length sequence of a wild-type TcBuster transposase (SEQ ID NO: 681). In some embodiments, the mutant TcBuster transposase comprises an amino acid sequence having at least 98%, at least 98.5%, at least 99%, at least 99.1%, at least 99.2%, at least 99.3%, at least 99.4%, at least 99.5%, at least 99.6%, at least 99.7%, at least 99.8%, at least 99.9% sequence identity to full length sequence of a wild-type TcBuster transposase (SEQ ID NO: 681). In some embodiments, the mutant TcBuster transposase comprises an amino acid sequence having at least one amino acid that is different from the full-length sequence of a wild-type TcBuster transposase (SEQ ID NO: 681). In some embodiments, the mutant TcBuster transposase comprises an amino acid sequence having at least 2, at least 3, at least 4, at least 5, at least 6, at least 7, at least 8, at least 9, at least 10, at least 11 or more amino acids that are different from the full-length sequence of a wild-type TcBuster transposase (SEQ ID NO: 681). In some embodiments, the mutant TcBuster transposase comprises an amino acid sequence having at least 5, at least 10, at least 20, at least 30, at least 40, at least 50, at least 60, at least 70, at least 80, at least 90, at least 100, at least 200, or at least 250 amino acids that are different from the full-length sequence of a wild-type TcBuster transposase (SEQ ID NO: 681). In some embodiments, the mutant TcBuster transposase comprises an amino acid sequence having at most 3, at most 6, at most 12, at most 25, at most 35, at most 45, at most 55, at most 65, at most 75, at most 85, at most 95, at most 150, or at most 250 amino acids that are different from the full-length sequence of a wild-type TcBuster transposase (SEQ ID NO: 681).

In some embodiments, a mutant TcBuster transposase of the disclosure comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30) amino acid substitutions in an amino acid residue selected from Q82, N85, D99, D132, Q151, E153, A154, Y155, E159, T171, K177, D183, D189, E263, E274, 5277, N281, L282, K292, V297, K299, A303, H322, A332, A358, D376, V377, L380, 1398, F400, V431, 5447, N450, 1452, E469, K469, P510, E519, R536, V553, P554, P559, K573, E578, K590, Y595, V596, T598, K599, Q615, T618, D622, E274, V549, R574, E570, G558, P554, D555, G556, L539, E538, E534, 1532, L564, T554, D555, T556, T557, K635, D607, Y595, S591, V583, E578, K573, T544, D545, T546, T547, Y59, G75, L76, S87, H124, D132, D133, C172, D189, T190, Y201, V206, N209, T219, A229, A229, I233, F237, M250, A255, P257, L268, K275, S277, Y284, H285, K292, C318, and H322 (amino acid residue positions in reference to SEQ ID NO: 681). In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30, or more) amino acid substitutions selected from Q82E, N85S, D99A, D132A, Q151S, Q151A, E153K, E153R, A154P, Y155H, E159A, T171K, T171R, K177E, D183K, D183R, D189A, E263A, E263K, E263R, E274K, E274R, S277K, N281E, L282K, L282R, K292P, V297K, K299S, A303T, H322E, A332S, A358E, A358K, A358S, D376A, V377T, L380N, I398D, I398S, 1398K, F400L, V431L, S447E, N450K, N450R, I452F, E469K, K469K, P510D, P510N, E519R, R536S, V553S, P554T, P559D, P559S, P559K, K573E, E578L, K590T, Y595L, V596A, T598I, K599A, Q615A, T618K, T618R, D622K, D622R, E274K, V549P, R574K, E570V, G558T, P554T, D555M, G556P, L539F, E538Q, E534A, 1532E, L564C, T554N, D555S, T556D, T557A, K635P, D6071, Y595A, S591I, V583P, E578L, K573R, T544N, D545S, T546D, T547A, Y59F, G75P, L76Q, S87E, H124D, D132K, D133L, C172V, D189N, T190N, T190D, Y201D, V206Q, N209E, T219S, A229S, A229D, I233Q, F237Y, M250F, A255P, P257E, L268T, K275E, 5277G, S277K, Y284I, H285G, K292N, C318I, H322Q, and H322A (amino acid residue positions in reference to SEQ ID NO: 681). In some embodiments, the mutant TcBuster transposase of the disclosure comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30) amino acid substitutions in an amino acid residue selected from Q82, N85, D99, D132, Q151, E153, A154, Y155, E159, T171, K177, D183, D189, E263, E274, 5277, N281, L282, K292, V297, K299, A303, H322, A332, A358, D376, V377, L380, 1398, F400, V431, S447, N450, I452, E469, K469, P510, E519, R536, V553, P554, P559, K573, E578, K590, Y595, V596, T598, K599, Q615, T618, D622, and E274 (amino acid residue positions in reference to SEQ ID NO: 681). In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30, or more) amino acid substitutions selected from Q82E, N85S, D99A, D132A, Q151S, Q151A, E153K, E153R, A154P, Y155H, E159A, T171K, T171R, K177E, D183K, D183R, D189A, E263A, E263K, E263R, E274K, E274R, S277K, N281E, L282K, L282R, K292P, V297K, K299S, A303T, H322E, A332S, A358E, A358K, A358S, D376A, V377T, L380N, I398D, I398S, I398K, F400L, V431L, S447E, N450K, N450R, I452F, E469K, K469K, P510D, P510N, E519R, R536S, V553S, P554T, P559D, P559S, P559K, K573E, E578L, K590T, Y595L, V596A, T598I, K599A, Q615A, T618K, T618R, D622K, D622R, and E274K (amino acid residue positions in reference to SEQ ID NO: 681). In some embodiments, a mutant TcBuster transposase of the disclosure comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30) amino acid substitutions in an amino acid residue selected from V549, R574, E570, G558, P554, D555, G556, L539, E538, E534, 1532, L564, T554, D555, T556, T557, K635, D607, Y595, S591, V583, E578, K573, T544, D545, T546, T547, Y59, G75, L76, S87, H124, D132, D133, C172, D189, T190, Y201, V206, N209, T219, A229, A229, 1233, F237, M250, A255, P257, L268, K275, S277, Y284, H285, K292, C318, H322, and H322 (amino acid residue positions in reference to SEQ ID NO: 681). In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30, or more) amino acid substitutions selected from V549P, R574K, E570V, G558T, P554T, D555M, G556P, L539F, E538Q, E534A, 1532E, L564C, T554N, D555S, T556D, T557A, K635P, D6071, Y595A, 55911, V583P, E578L, K573R, T544N, D545S, T546D, T547A, Y59F, G75P, L76Q, S87E, H124D, D132K, D133L, C172V, D189N, T190N, T190D, Y201D, V206Q, N209E, T219S, A229S, A229D, I233Q, F237Y, M250F, A255P, P257E, L268T, K275E, S277G, S277K, Y284I, H285G, K292N, C318I, H322Q, and H322A (amino acid residue positions in reference to SEQ ID NO: 681).

In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30, or more) amino acid substitutions, or combinations of substitutions in an amino acid residue or combination of amino acid residues selected from V377 and E469; V377, E469, and R536S; A332; V553 and P554; E519; K299; Q615 and T618; 5277; A303; P510; N281; K590; E274; Q258; E247; 5447; N85; V297; A358; 1452; V377, E469, and D189; K573 and E578; 1452, V377, E469, and D189; A358, V377, E469, and D189; K573, E578, V377, E469, and D189; T171; D183; S193; P257; E263; L282; T618; D622; E153, N450; T171; D183; S193; P257; E263; L282; T618; D622; E153; N450; and E247, E274, V297, and A358 (amino acid residue positions in reference to SEQ ID NO: 681).

In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30, or more) amino acid substitutions, or combinations of substitutions selected from V377T/E469K; V377T/E469K/R536S; A332S; V553S/P554T; E519R; K299S; Q615A/T618K; S277K; A303T; P510D; P510N; N281S; N281E; K590T; E274K; Q258T; E247K; S447E; N85S; V297K; A358K; I452F; V377T/E469K/D189A; K573E/E578L; 1452F/V377T/E469K/D189A; A358K/V377T/E469K/D189A; K573E/E578L/V377T/E469K/D189A; T171R; D183R; S193R; P257K; E263R; L282K; T618K; D622R; E153K; N450K; T171K; D183K; S193K; P257R; E263K; L282R; T618R; D622K; E153R; N450R; and E247K/E274K/V297K/A358K (amino acid residue positions in reference to SEQ ID NO: 681). In some embodiments, the mutant TcBuster transposase comprises a substitution of an aspartic acid at position 189 with an alanine (D189A); a valine at position 377 with a threonine (V377T); and a glutamic acid at position 469 with a lysine (E469K).

In some embodiments, the mutant TcBuster transposase comprises one or more amino (e.g., at least one, at least 2, at least 3, at least 4, at least 5, at least 6, at least 7, at least 8, at least 9, at least 10, at least 11, at least 12, at least 13, at least 14, at least 15, at least 20, at least 30, or more) acid substitutions, or combinations of substitutions in an amino acid residue or combination of amino acid residues selected from V377 and E469; V377, E469, and R536S; A332; V553 and P554; E519; K299; Q615 and T618; S277; A303; P510; N281; K590; E274; Q258; E247; S447; N85; V297; A358; I452; V377, E469, and D189; and K573 and E578 (amino acid residue positions in reference to SEQ ID NO: 681). In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30, or more) amino acid substitutions, or combinations of substitutions selected from V377T/E469K; V377T/E469K/R536S; A332S; V553S/P554T; E519R; K299S; Q615A/T618K; S277K; A303T; P510D; P510N; N281S; N281E; K590T; E274K; Q258T; E247K; S447E; N85S; V297K; A358K; I452F; V377T/E469K/D189A; and K573E/E578L (amino acid residue positions in reference to SEQ ID NO: 681).

In some embodiments, the mutant TcBuster transposase is a hyperactive mutant TcBuster transposase. A “hyperactive” mutant TcBuster transposase, as used herein, can refer to any mutant TcBuster transposase that has increased transposition efficiency as compared to a wild-type TcBuster transposase having amino acid sequence SEQ ID NO: 681. In non-limiting examples, when compared to a wild-type TcBuster transposase, a hyperactive mutant TcBuster transposase may have (i) better transposition efficiency when the temperature is higher than normal cell culture temperature; (ii) better transposition efficiency in a relative acidic or basic aqueous medium; and/or (iii) better transposition efficiency when a particular type of transfection technique (e.g., electroporation) is performed. Hyperactive mutant TcBuster transposase may be generated by systemically mutating amino acids of TcBuster transposase to increase a net charge of the amino acid sequence. In some embodiments, this method comprises performing systematic alanine scanning to mutate aspartic acid (D) or glutamic acid (E), which are negatively charged at a neutral pH, to alanine residues. In some embodiments, this method comprises performing systematic mutation to lysine (K) or arginine (R) residues, which are positively charged at a neutral pH.

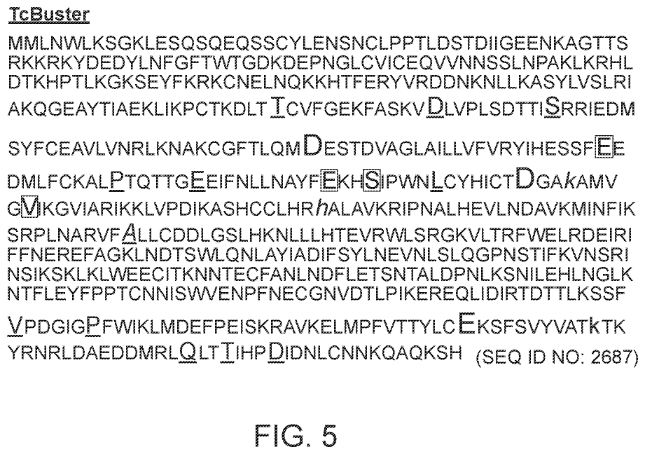

Without wishing to be bound by theory, an increase in a net charge of the amino acid sequence at a neutral pH may increase the transposition efficiency of the TcBuster transposase. Particularly, when the net charge is increased in proximity to a catalytic domain of the transposase, the transposition efficiency is expected to increase. It can be contemplated that positively charged amino acids can form points of contact with a DNA target and allow the catalytic domains to act on the DNA target. It may also be contemplated that loss of these positively charged amino acids can decrease either excision or integration activity in transposases. FIG. 5 depicts the wild type TcBuster transposase amino acid sequence, highlighting amino acids that may be points of contact with DNA. An exemplary method of the present disclosure comprises mutating amino acids of the TcBuster transposase that are predicted to be in close proximity to, or to make direct contact with, the DNA. These amino acids can be substituted with amino acids identified as being conserved in other member(s) of the hAT family (e.g., other members of the Buster and/or Ac subfamilies). The amino acids predicted to be in close proximity to, or to make direct contact with, the DNA can be identified, for example, by reference to a crystal structure, predicted structures, mutational analysis, functional analysis, alignment with other members of the hAT family, or any other suitable method.

In some embodiments, a mutant TcBuster transposase comprises one or more amino acid substitutions that increase a net charge at a neutral pH in comparison to SEQ ID NO: 681. In some embodiments, a mutant TcBuster transposase comprising one or more amino acid substitutions that increase a net charge at a neutral pH in comparison to SEQ ID NO: 681 can be hyperactive. In some embodiments, the mutant TcBuster transposase comprises one or more substitutions to a positively charged amino acid, such as, but not limited to, lysine (K) or arginine (R). In some embodiments, the mutant TcBuster transposase comprises one or more substitutions of a negatively charged amino acid, such as, but not limited to, aspartic acid (D) or glutamic acid (E), with a neutral amino acid, or a positively charged amino acid.

A non-limiting example of a mutant TcBuster useful in the compositions and methods of the disclosure is a mutant TcBuster transposase that comprises one or more amino acid substitutions that increase a net charge at a neutral pH within or in proximity to a catalytic domain in comparison to SEQ ID NO: 681. The catalytic domain can be the first catalytic domain or the second catalytic domain. The catalytic domain can also include both catalytic domains of the transposase.

Without wishing to be bound by theory, TcBuster transposase, like other members of the hAT transposase family, comprises a DDE motif, which may be the active site that catalyzes the movement of the transposon. It is contemplated that D223, D289, and E589 make up the active site, which is a triad of acidic residues. The DDE motif may coordinate divalent metal ions and can be important in the catalytic reaction. In some embodiments, a mutant TcBuster transposase comprises one or more amino acid substitutions that increase a net charge at a neutral pH in comparison to SEQ ID NO: 681, and the one or more amino acids are located in proximity to D223, D289, or E589, when numbered in accordance to SEQ ID NO: 681. In some embodiments, a mutant TcBuster transposase as provided herein does not comprise any disruption of the catalytic triad, i.e., D223, D289, or E589. In some embodiments, the mutant TcBuster transposase does not comprise any amino acid substitution at D223, D289, or E589. In some embodiments, the mutant TcBuster transposase may comprise an amino acid substitution at D223, D289, or E589, but such substitution does not disrupt the catalytic activity contributed by the catalytic triad. In some embodiments, the term “proximity” can refer to a measurement of a linear distance in the primary structure of the transposase. For instance, the distance between D223 and D289 in the primary structure of a wild-type TcBuster transposase is 66 amino acids. In certain embodiments, the proximity can refer to a distance of about 70 to 80 amino acids. In some embodiments, the proximity can refer to a distance of about 80, 75, 70, 60, 50, 40, 30, 20, 10, or 5 amino acids. In some embodiments, the term “proximity” can refer to a measurement of a spatial relationship in the secondary or tertiary structure of the transposase, i.e. when the transposase folds into its three dimensional configurations. In some embodiments, the proximity can refer to a distance of about 1 Å, about 2 Å, about 5 Å, about 8 Å, about 10 Å, about 15 Å, about 20 Å, about 25 Å, about 30 Å, about 35 Å, about 40 Å, about 50 Å, about 60 Å, about 70 Å, about 80 Å, about 90 Å, or about 100 Å. A neutral pH can be a pH value around 7 (e.g., between 6.9 and 7.1, between 6.8 and 7.2, between 6.7 and 7.3, between 6.6 and 7.4, between 6.5 and 7.5, between 6.4 and 7.6, between 6.3 and 7.7, between 6.2-7.8, between 6.1-7.9, between 6.0-8.0, between 5-8, or in a range derived therefrom).

Non-limiting exemplary mutant TcBuster transposases that comprise one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30) amino acid substitutions that increase a net charge at a neutral pH in comparison to SEQ ID NO: 681 include TcBuster transposases at an amino acid residue selected from E247, E274, V297, A358, S277, E247, E274, V297, A358, S277, T171, D183, S193, P257, E263, L282, T618, D622, E153, N450, T171, D183, 5193, P257, E263, L282, T618, D622, E153, D132, S277, L359, N417, Y427, S591, and Q615 (amino acid residue positions in reference to SEQ ID NO: 681). In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20, at least 30) amino acid substitutions selected from E247K, E274K, V297K, A358K, S277K, E247R, E274R, V297R, A358R, S277R, T171R, D183R, S193R, P257K, E263R, L282K, T618K, D622R, E153K, N450K, T171K, D183K, S193K, P257R, E263K, L282R, T618R, D622K, E153R, and N450R (amino acid residue positions in reference to SEQ ID NO: 681).

In some embodiments, a mutant TcBuster transposase comprises one or more amino acid substitutions that increase a net charge at a non-neutral pH in comparison to SEQ ID NO: 681. In some embodiments, the net charge is increased by one or more amino acid substitutions within or in proximity to a catalytic domain at a non-neutral pH. In some embodiments, the net charge is increased by one or more amino acid substitutions in proximity to D223, D289, or E589, at a non-neutral pH. In some embodiments, the non-neutral pH can be a pH value lower than 7, lower than 6.5, lower than 6, lower than 5.5, lower than 5, lower than 4.5, lower than 4, lower than 3.5, lower than 3, lower than 2.5, lower than 2, lower than 1.5, or lower than 1. In other embodiments, the non-neutral pH can also be a pH value higher than 7, higher than 7.5, higher than 8, higher than 8.5, higher than 9, higher than 9.5, or higher than 10.

In some embodiments, the disclosure provides a method of systemically mutating amino acids in the DNA binding and oligomerization domains of the TcBuster transposase. Without wishing to be bound by theory, mutation in the DNA binding and oligomerization domain may increase the binding affinity to DNA target and promote oligomerization activity of the TcBuster transposase, which consequentially may promote transposition efficiency. More specifically, the method comprises systemically mutating amino acids one by one within or in proximity to the DNA binding and oligomerization domain (e.g., amino acid 112 to 213). The method may also comprise mutating more than one amino acid within or in proximity to the DNA binding and oligomerization domain. The method may also comprise mutating one or more amino acids within or in proximity to the DNA binding and oligomerization domain, together with one or more amino acids outside the DNA binding and oligomerization domain.

In some embodiments, the method comprises performing rational replacement of selective amino acid residues based on multiple sequence alignments of TcBuster with other hAT family transposases (Ac, Hermes, Hobo, Tag2, Tam3, Hermes, Restless and Tol2) or with other members of Buster subfamily (e.g., AeBuster1, AeBuster2, AeBuster3, BtBuster1, BtBuster2, CfBuster1, and CfBuster2). Without being bound by a certain theory, conservancy of certain amino acids among other hAT family transposases, especially among the active ones, may indicate their importance for the catalytic activity of the transposases. Therefore, replacement of unconserved amino acids in wild-type TcBuster sequence (SEQ ID NO: 681) with conserved amino acids among other hAT family may yield a hyperactive mutant TcBuster transposase. The method may comprise obtaining sequences of TcBuster as well as other hAT family transposases; aligning the sequences and identifying the amino acids in TcBuster transposase with a different conserved counterpart among the other hAT family transposases; and performing site-directed mutagenesis to produce mutant TcBuster transposase harboring the mutation(s).

In some embodiments, a hyperactive mutant TcBuster transposase comprises one or more amino acid substitutions based on alignment to other members of Buster subfamily or other members of hAT family. In some embodiments, the one or more amino acid substitutions can be substitutions of conserved amino acid for the unconserved amino acid in wild-type TcBuster sequence (SEQ ID NO: 681). Non-limiting examples of mutant TcBuster transposases include TcBuster transposases that comprise one or more (e.g., at least one, at least 2, at least 3, at least 4, at least 5, at least 6, at least 7, at least 8, at least 9, at least 10, at least 11, at least 12, at least 13, at least 14, at least 15, at least 20) amino acid substitutions in an amino acid residue selected from Q151, A154, Q615, V553, Y155, Y201, F202, C203, F400, 1398, V431, Y59, G75, L76, S87, H124, D133, C172, D189, D190, T190, Y201, V206, N209, T219, A229, 1233, F237, M250, A255, P257, L268, K275, 5277, Y284, H285, K292, C318, H322, M343, A354, G365, F389, Y427, S426, C462, C470, A472, N473, K490, S491, N492, E535, R536, E538, E567, F568, R574, R574, R574, K590, V594, M612, A632, Y155, 1421, A632, P559, G526, C512, V356, Y284, and N90 (amino acid residue positions in reference to SEQ ID NO: 681). In some embodiments, the mutant TcBuster transposase comprises one or more (e.g., at least one, at least 2, at least 3, at least 4, at least 5, at least 6, at least 7, at least 8, at least 9, at least 10, at least 11, at least 12, at least 13, at least 14, at least 15, at least 20) amino acid substitution selected from Q151S, Q151 Å, A154P, Q615 Å, V553S, Y155H, Y201 Å, F202D, F202K, C203I, C203V, F400L, I398D, 1398S, I398K, V431L, Y59F, G75P, L76Q, S87E, H124D, D133L, C172V, D189N, T190N, T190D, Y201D, V206Q, N209E, T219S, A229S, A229D, I233Q, F237Y, M250F, A255P, P257E, L268T, K275E, S277G, Y284I, H285G, K292N, C318I, H322Q, H322 Å, M343L, A354S, G365D, F389V, Y427S, S426Q, C462D, C470M, A472P, A472D, N473T, K490I, S491N, N492G, E535 Å, R536Q, E538 Å, E567S, F568Y, R574E, R574I, R574T, K590 Å, V594S, M612L, M612S, A632S, Y155F, I421L, A632Q, P559I, G526V, C512E, V356L, Y284V, and N90S (amino acid residue positions in reference to SEQ ID NO: 681).

Another method of generating mutant TcBuster transposases comprises systemically mutating acidic amino acids to basic amino acids and identifying a resulting hyperactive mutant transposase. In some embodiments, the mutant TcBuster transposase comprises amino acid substitutions V377T, E469K, and D189 Å. In some embodiments, a mutant TcBuster transposase comprises amino acid substitutions K573E and E578L. In some embodiments, a mutant TcBuster transposase comprises amino acid substitution I452K. In some embodiments, a mutant TcBuster transposase comprises amino acid substitution A358K. In some embodiments, a mutant TcBuster transposase comprises amino acid substitution V297K. In some embodiments, a mutant TcBuster transposase comprises amino acid substitution N85S. In some embodiments, a mutant TcBuster transposase comprises amino acid substitutions N85S, V377T, E469K, and D189 Å. In some embodiments, a mutant TcBuster transposase comprises amino acid substitutions I452F, V377T, E469K, and D189 Å. In some embodiments, a mutant TcBuster transposase comprises amino acid substitutions A358K, V377T, E469K, and D189 Å. In some embodiments, a mutant TcBuster transposase comprises amino acid substitutions V377T, E469K, D189 Å, K573E and E578L.

### Inverted Terminal Repeats (ITRs)

A transposon generally comprises two ITR nucleotide sequences. A transposon described herein may be engineered to comprise a cargo cassette comprising two ITR sequences. In some embodiments, at least one of the ITRs contains at least one direct repeat. In some embodiments, the transposase is one or more of the TcBuster transposases (e.g., mutant TcBuster transposases) disclosed herein, and the TcBuster transposase recognizes one or more ITRs disclosed in Table 10. In some embodiments, a transposon may contain a cargo cassette comprising the nucleic acid sequences of IRDR-L-Seq1 (SEQ ID NO: 2662) and IRDR-R-Seq1 (SEQ ID NO: 2663). The terms “left” and “right”, as used herein with respect to inverted repeats, can refer to the 5′ and 3′ sides or ends of the cargo cassette on the sense strand of the double strand transposon, respectively. In some embodiments, a left inverted repeat can comprise a nucleic acid sequence at least 50%, 60%, 70%, 80%, 85%, 90%, 95%, 96%, 97%, 98%, 99%, or 100% identical to IRDR-L-Seq1 (SEQ ID NO: 2662). In some embodiments, a right inverted repeat can comprise a nucleic acid sequence at least 50%, 60%, 70%, 80%, 85%, 90%, 95%, 96%, 97%, 98%, 99%, or 100% identical to IRDR-R-Seq1 (SEQ ID NO: 2663). In other embodiments, a right inverted repeat can comprise a sequence at least 50%, 60%, 70%, 80%, 85%, 90%, 95%, 96%, 97%, 98%, 99%, or 100% identical to IRDR-L-Seq1 (SEQ ID NO: 2662). In some embodiments, a left inverted repeat can comprise a sequence at least 50%, 60%, 70%, 80%, 85%, 90%, 95%, 96%, 97%, 98%, 99%, or 100% identical to IRDR-R-Seq1 (SEQ ID NO: 2663).

In other embodiments, the transposon may comprise a cargo cassette comprising the ITR sequences of IRDR-L-Seq2 (SEQ ID NO: 2664) and IRDR-R-Seq2 (SEQ ID NO: 2665). In some embodiments, a left inverted repeat can comprise a nucleic acid sequence at least 50%, 60%, 70%, 80%, 85%, 90%, 95%, 96%, 97%, 98%, 99%, or 100% identical to IRDR-L-Seq2 (SEQ ID NO: 2664). In some embodiments, a right inverted repeat can comprise a nucleic acid sequence at least 50%, 60%, 70%, 80%, 85%, 90%, 95%, 96%, 97%, 98%, 99%, or 100% identical to IRDR-R-Seq2 (SEQ ID NO: 2665). In other embodiments, a right inverted repeat can comprise a nucleic acid sequence at least 50%, 60%, 70%, 80%, 85%, 90%, 95%, 96%, 97%, 98%, 99%, or 100% identical to IRDR-L-Seq2 (SEQ ID NO: 2664). In some embodiments, a left inverted repeat can comprise a nucleic acid sequence at least 50%, 60%, 70%, 80%, 85%, 90%, 95%, 96%, 97%, 98%, 99%, or 100% identical to IRDR-R-Seq2 (SEQ ID NO: 2665).

Alternatively, a transposon can comprise a cargo cassette comprising the nucleotide sequences of IRDR-L-Seq3 (SEQ ID NO: 2666) and IRDR-R-Seq3 (SEQ ID NO: 2667). In some embodiments, a left inverted repeat can comprise a nucleic acid sequence at least 50%, 60%, 70%, 80%, 85%, 90%, 95%, 96%, 97%, 98%, 99%, or 100% identical to IRDR-L-Seq3 (SEQ ID NO: 2666). In some embodiments, a right inverted repeat can comprise a nucleic acid sequence at least 50%, 60%, 70%, 80%, 85%, 90%, 95%, 96%, 97%, 98%, 99%, or 100% identical to IRDR-R-Seq3 (SEQ ID NO: 2667). In other embodiments, a right inverted repeat can comprise a nucleic acid sequence at least 50%, 60%, 70%, 80%, 85%, 90%, 95%, 96%, 97%, 98%, 99%, or 100% identical to IRDR-L-Seq3 (SEQ ID NO: 2666). In some embodiments, a left inverted repeat can comprise a nucleic acid sequence at least 50%, 60%, 70%, 80%, 85%, 90%, 95%, 96%, 97%, 98%, 99%, or 100% identical to IRDR-R-Seq3 (SEQ ID NO: 2667).

A transposon may comprise a cargo cassette comprising two inverted repeats that have different nucleotide sequences than the sequences in Table 10, or a combination of the various sequences known to one skilled in the art. In some embodiments, at least one of the two inverted repeats of a transposon provided herein may contain a nucleic acid sequence at least 80%, 85%, 90%, 95%, 96%, 97%, 98%, 99%, or 100% identical to any one of SEQ ID NOs: 2662, 2663, 2664, 2665, 2666 and 2667. In some embodiments, at least one of inverted repeats of a transposon provided herein may contain a sequence that is at least 80%, 85%, 90%, 95%, 96%, 97%, 98%, or 99% identical to SEQ ID NO: 2662. In some embodiments, at least one of inverted repeats of a transposon provided herein may contain a sequence that is at least 80%, 85%, 90%, 95%, 96%, 97%, 98%, or 99% identical to SEQ ID NO: 2663. In some embodiments, at least one of inverted repeats of a transposon provided herein may contain a sequence that is at least 80%, 85%, 90%, 95%, 96%, 97%, 98%, or 99% identical to SEQ ID NO: 2664. In some embodiments, at least one of inverted repeats of a transposon provided herein may contain a sequence that is at least 80%, 85%, 90%, 95%, 96%, 97%, 98%, or 99% identical to SEQ ID NO: 2665. In some embodiments, at least one of inverted repeats of a transposon provided herein may contain a sequence that is at least 80%, 85%, 90%, 95%, 96%, 97%, 98%, or 99% identical to SEQ ID NO: 2666. In some embodiments, at least one of inverted repeats of a transposon provided herein may contain a sequence that is at least 80%, 85%, 90%, 95%, 96%, 97%, 98%, or 99% identical to SEQ ID NO: 2667. The choice of inverted repeat sequences may vary depending on the expected transposition efficiency, the type of cell to be modified, the transposase to use, and many other factors. In some embodiments, minimally sized transposon vector inverted terminal repeats that conserve genomic space may be used. The ITRs of hAT family transposons diverge greatly with differences in right-hand and left-hand ITRs. In some embodiments, smaller ITRs consisting of just 100-200 nucleotides are as active as the longer native ITRs in hAT transposon vectors. These sequences may be consistently reduced while mediating hAT family transposition. These shorter ITRs can conserve genomic space within hAT transposon vectors.

The inverted repeats of a transposon provided herein can be about 50 to 2000 nucleotides, about 50 to 1000 nucleotides, about 50 to 800 nucleotides, about 50 to 600 nucleotides, about 50 to 500 nucleotides, about 50 to 400 nucleotides, about 50 to 350 nucleotides, about 50 to 300 nucleotides, about 50 to 250 nucleotides, about 50 to 200 nucleotides, about 50 to 180 nucleotides, about 50 to 160 nucleotides, about 50 to 140 nucleotides, about 50 to 120 nucleotides, about 50 to 110 nucleotides, about 50 to 100 nucleotides, about 50 to 90 nucleotides, about 50 to 80 nucleotides, about 50 to 70 nucleotides, about 50 to 60 nucleotides, about 75 to 750 nucleotides, about 75 to 450 nucleotides, about 75 to 325 nucleotides, about 75 to 250 nucleotides, about 75 to 150 nucleotides, about 75 to 95 nucleotides, about 100 to 500 nucleotides, about 100 to 400 nucleotides, about 100 to 350 nucleotides, about 100 to 300 nucleotides, about 100 to 250 nucleotides, about 100 to 220 nucleotides, or about 100 to 200 nucleotides in length, or any range having upper and lower values derived from any of the foregoing recited values, e.g., from about 50 to 75 nucleotides.

TABLE 10
Exemplary Inverse Terminal Repeats (ITRs)
Recognized by TcBuster Transposase

| ITR | SEQ ID NO |
|---|---|
| IRDR-L-Seq1 | SEQ ID NO: 2662 |
| IRDR-R-Seq1 | SEQ ID NO: 2663 |
| IRDR-L-Seq2 | SEQ ID NO: 2664 |
| IRDR-R-Seq2 | SEQ ID NO: 2665 |
| IRDR-L-Seq3 | SEQ ID NO: 2666 |
| IRDR-R-Seq3 | SEQ ID NO: 2667 |

Cargo Nucleotide Sequences and Cargo Cassettes

In some embodiments, the disclosure provides a nucleic acid molecule comprising a cargo nucleotide sequence encoding a CAR described herein and optionally a functional effector element (e.g., a cytokine). In some embodiments, the disclosure provides a nucleic acid molecule comprising a) a first nucleic acid sequence; and b) a second nucleic acid sequence; wherein the first nucleic acid sequence encodes a CAR described herein.

In some embodiments, the first nucleic acid is located upstream of the second nucleic acid. In some embodiments, the first nucleic acid is located downstream of the second nucleic acid.

In some embodiments, the first nucleic acid further comprises a first promoter sequence capable of expressing an exogenous sequence in a human cell. In some embodiments, the first promoter sequence is a constitutive promoter. In some embodiments, the first promoter sequence is an inducible promoter. In some embodiments, first promoter sequence is an EF1, EF1alpha, EFS, MND, PGK, CMV IE, dectin-1, dectin-2, CD1 lc, F4/80, SM22, RSV, SV40, Ad MLP, beta-actin, MHC class I, MHC class II, U6 or H1 promoter. In some embodiments, the first promoter sequence is EF1a promoter. In some embodiments, the first promoter sequence is MND promoter.

The cargo nucleotide sequence may comprise any nucleotide sequence described herein, e.g., a nucleotide sequence intended for integration into acceptor DNA and/or a nucleotide sequence encoding for one or more polypeptides intended to be expressed or produced in an immune cell, e.g., an NK cell. In some embodiments, the cargo nucleotide sequence comprises a nucleotide sequence that encodes for a CAR, a cytokine, and/or a chimeric TGF-β protein described herein. The disclosure further provides a nucleic acid molecule comprising a cargo nucleotide sequence comprising any nucleotide sequence described herein, e.g., a nucleotide sequence intended for integration into acceptor DNA and/or a nucleotide sequence encoding for one or more polypeptides intended to be expressed or produced in an immune cell, e.g., an NK cell (e.g., a nucleic acid sequence encoding for a CAR, a cytokine, and/or a chimeric TGF-β protein described herein).

In some embodiments, the first nucleic acid sequence further encodes an additional exogenous polypeptide, wherein the sequence encoding the additional exogenous polypeptide is located downstream of the nucleic acid sequence encoding the CAR. In some embodiments, the additional exogenous polypeptide is an IL-15, an IL-15Ra-binding fragment of IL-15, a membrane bound IL-15 (mbIL-15), an IL-15 receptor alpha (IL-15Rα), a fusion of IL-15 and IL-15Ra, a co-stimulatory molecule, a TGFbeta signal converter, a PEBL element and/or a second CAR comprising an antigen recognition domain that specifically binds an antigen other than human CD70. In some embodiments, the additional exogenous polypeptide comprises a TGFbeta signal converter.

In some embodiments, the cargo nucleotide sequence comprises a nucleotide sequence encoding one or more of (a) a chimeric protein comprising an extracellular domain, a transmembrane domain, and an intracellular domain, wherein the extracellular domain binds to TGF-β, and wherein the intracellular domain comprises an intracellular domain, or a portion thereof, of a stimulatory polypeptide; (b) a chimeric antigen receptor (CAR); and/or (c) a cytokine, e.g., a membrane-associated IL-15/IL-15RA. In some embodiments, the CAR comprises a CD70 antigen binding domain.

In some embodiments, the cargo nucleotide sequence comprises a nucleotide sequence encoding one or more of (a) a protein comprising a dominant-negative isoform of a TGF-BR2, wherein the dominant-negative isoform of TGF-BR21 competes with a wild-type isoform of a TGF-BR2 for binding TGF-B; (b) a chimeric antigen receptor (CAR); and/or (c) a cytokine, e.g., a membrane-associated IL-15/IL-15RA. In some embodiments, the CAR comprises a CD70 antigen binding domain.

In some embodiments, the second nucleic acid sequence of a cargo nucleotide sequence encodes an shRNA. In some embodiments, the second nucleic acid sequence encodes an shRNA of SEQ ID NO: 2647, 2648, 2649, 2650, 2651 or 2652. In some embodiments, the second nucleic acid sequence comprises a sequence of SEQ ID NO: 2656, 2657, 2658, 2659, 2660 or 2661.

In some embodiments, the second nucleic acid further comprises a second promoter sequence capable of expressing an exogenous sequence in a human cell. In some embodiments, the second promoter sequence is a constitutive promoter. In some embodiments, the second promoter sequence is an inducible promoter. In some embodiments, the second promoter sequence is an EF1, EF1alpha, EFS, MND, PGK, CMV IE, dectin-1, dectin-2, human CD11c, F4/80, SM22, RSV, SV40, Ad MLP, beta-actin, MHC class I, MHC class II, U6 or H1 promoter. In some embodiments, the second promoter sequence is a U6 promoter comprising SEQ ID NO: 2653.

In some embodiments, the disclosure provides a nucleic acid molecule comprising a) a first nucleic acid sequence; and b) a second nucleic acid sequence; wherein the first nucleic acid sequence and the second nucleic acid sequence are located between a first terminal repeat (TR) sequence and a second TR sequence. In some embodiments, the first nucleic acid sequence encodes a CAR described herein. In some embodiments, the first TR sequence is a first inverted terminal repeat (ITR) sequence and the second TR sequence is a second ITR sequence. In some embodiments, the first TR sequence is a first long terminal repeat (LTR) sequence and the second TR sequence is a second LTR sequence.

In some embodiments, the disclosure provides a viral-vector related nucleic acid molecule, wherein the nucleic acid molecule is engineered to comprise a cargo cassette comprising viral LTR nucleotide sequences flanking a cargo nucleotide sequence.

In some embodiments, the disclosure provides a transposon-related nucleic acid molecule, wherein the nucleic acid molecule is engineered to comprise a cargo cassette comprising ITR nucleotide sequences flanking a cargo nucleotide sequence. The ITR nucleotide sequences are recognized by a transposase. The transposase and related ITR nucleotide sequences may be from any transposon/transposase system described herein.

The disclosure further provides a nucleic acid molecule comprising a nucleotide sequence of a first ITR, a nucleotide sequence of a second ITR, and a cargo nucleotide sequence, i.e., a nucleotide sequence encoding for one or more polypeptides intended to be expressed or produced in an immune cell, e.g., an NK cell. In some embodiments, the polypeptide is a CAR, a cytokine, and/or a chimeric TGF-β protein described herein. In some embodiments, the first and second ITRs are any two of the ITR nucleotide sequences provided in Table 10. In some embodiments, the first and second ITRs are IRDR-L-Seq3 and IRDR-R-Seq3, respectively. In some embodiments, the first and second ITRs flank the cargo nucleotide sequence.

In some embodiments, the cargo cassette, or nucleic acid sequence comprising a first TR nucleotide sequence, a second TR nucleotide sequence, and a cargo nucleotide sequence, is present in an expression vector. The expression vector can be selected from any of the vectors disclosed herein, or any other vectors known to one skilled in the art. In some embodiments, the expression vector is a viral vector. In some embodiments, the viral vector is a lentiviral vector or a gamma-retroviral vector. In some embodiments, the expression vector is a DNA plasmid. In some embodiments the expression vector is a mini-circle vector. In some embodiments, the expression vector is a nanoplasmid vector. In some embodiments, the nanoplasmid vector is selected from the vectors NTC9385C (SEQ ID NO: 2668), NTC9685C (SEQ ID NO: 2669), NTC9385R (SEQ ID NO: 2670), and NTC9685R (SEQ ID NO: 2671), and modifications thereof, as described in International PCT Publication Nos. WO2014/035457 and WO2019/183248, each of which is incorporated in its entirety herein by reference.

In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2668. In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2669. In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2670. In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2671. Nanoplasmid vectors suitable for use in the present disclosure are described in further detail herein.

Polynucleotides Encoding the Transposase System

One aspect of the present disclosure provides a polynucleotide comprising a nucleotide sequence that encodes for a transposase described herein. In some embodiments, the polynucleotide further comprises a nucleotide sequence of a transposon (e.g., an engineered transposon) recognizable by the transposase. In some embodiments, the polynucleotide is comprised in an expression vector. In some embodiments, the expression vector is a DNA plasmid. In some embodiments, the expression vector is a mini-circle vector. In some embodiments, the expression vector is a nanoplasmid.

The term “mini-circle vector” as used herein can refer to a small circular plasmid derivative that is free of most, if not all, prokaryotic vector parts (e.g., control sequences or non-functional sequences of prokaryotic origin).

In some embodiments, the mini-circle vector comprises a TcBuster transposon. In some embodiments, the TcBuster transposon can have a size about1.5 kb, about 2 kb, about 2.2 kb, about 2.4 kb, about 2.6 kb, about 2.8 kb, about 3 kb, about 3.2 kb, about 3.4 kb, about 3.6 kb, about 3.8 kb, about 4 kb, about 4.2 kb, about 4.4 kb, about 4.6 kb, about 4.8 kb, about 5 kb, about 5.2 kb, about 5.4 kb, about 5.6 kb, about 5.8 kb, about 6 kb, about 6.5 kb, about 7 kb, about 8 kb, about 9 kb, about 10 kb, about 12 kb, about 25 kb, about 50 kb, or a value between any two of these numbers. In some embodiments, the TcBuster transposon can have a size of at most 2.1 kb, at most 3.1 kb, at most 4.1 kb, at most 4.5 kb, at most 5.1 kb, at most 5.5 kb, at most 6.5 kb, at most 7.5 kb, at most 8.5 kb, at most 9.5 kb, at most 11 kb, at most 13 kb, at most 15 kb, at most 30 kb, or at most 60 kb.

For genome editing applications with transposons, in some embodiments, it may be desirable to design a transposon for use in a binary system based on two distinct plasmids, whereby the nucleic acid sequence encoding for the transposase is physically separated from the transposon nucleic acid sequence containing the gene of interest flanked by the inverted repeats. Co-delivery of the transposon and transposase-encoding plasmids into the target cells enables transposition via a conventional cut-and-paste mechanism. In some other embodiments, a transposon based system as described herein may comprise a polynucleotide comprising both a nucleic acid sequence encoding a transposase as described herein, and a nucleic acid sequence of a transposon as described herein, i.e., wherein the nucleic acid encoding for the transposase and the transposon nucleic acid are present in the same plasmid.

One of the limitations of application of plasmid vectors is that transgene expression duration from plasmid vectors is reduced due to promoter inactivation mediated by the bacterial region (i.e., the region encoding the bacterial replication origin and selectable marker) of the vector (Chen et al., 2004. Gene Ther. 11:856-864; Suzuki et al., 2006. J Virol. 80:3293-3300). This results in short duration transgene expression. A strategy to improve transgene expression duration is to remove the bacterial region of the plasmid. For example, minicircle vectors have been developed which do not contain a bacterial region. Removal of the bacterial region in minicircle vectors improved transgene expression duration (Chen et al., 2004, supra). In minicircle vectors, the eukaryotic region polyadenylation signal is covalently linked to the eukaryotic region promoter through a short spacer typically less than 200 bp comprised of the recombined attachment sites. This linkage (spacer region) can tolerate a much longer spacer sequence since while long spacers>1 kb in length resulted in transgene expression silencing in vivo, shorter spacers<500 bp exhibited similar transgene expression patterns to conventional minicircle DNA vectors (Lu et al. Mol. Ther. 20:2111-9, 2012).

In some embodiments, a vector useful in various aspects of the disclosure is a nanoplasmid vector. The term “nanoplasmid vector” as used herein, refers to a vector combining an RNA selectable marker with a R6K, ColE2 or ColE2 related replication origin. Nanoplasmid vectors can be selected from the nanoplasmid vectors disclosed in any of International PCT Publication No. WO2014/035457, International PCT Publication No. WO2014/077866, and International PCT Publication No. WO2019/183248, each of which is incorporated in its entirety herein by reference.

In some embodiments, a vector useful in the present disclosure is selected from the vectors NTC8385, NTC8485 and NTC8685. NTC8385, NTC8485 and NTC8685 are antibiotic-free pUC origin vectors, which are precursors to nanoplasmid vectors, and contain a short RNA (RNA-OUT) selectable marker instead of an antibiotic resistance marker such as kanR. The creation and application of these RNA-OUT based antibiotic-free vectors is described in International PCT Publication No. WO2008/153733 and US Publication No. 2010/0184158, each of which is incorporated in its entirety herein by reference.

In some embodiments, a nanoplasmid vector useful in the present disclosure is selected from the vectors NTC9385C (SEQ ID NO: 2668), NTC9685C (SEQ ID NO: 2669), NTC9385R (SEQ ID NO: 2670), and NTC9685R (SEQ ID NO: 2671), and modifications thereof, as described in International PCT Publication No. WO2014/035457, which is incorporated in its entirety herein by reference. The NTC9385C nanoplasmid vector comprises a ColE2 Replication origin and a spacer region encoded bacterial region (replication and selection) of 281 bp [Nhel site-ssiA-ColE2 Origin (+7)-RNA-OUT-KpnI site]. The NTC9685C nanoplasmid vector comprises a ColE2 Replication origin, a spacer region encoded bacterial region (replication and selection) of 281 bp [Nhel site-ssiA-ColE2 Origin (+7)-RNA-OUT-KpnI site], and a VA1 RNA and SV40 enhancer. The NTC9385R nanoplasmid vector comprises a R6K Replication origin and a spacer region encoded bacterial region (replication and selection) of 466 bp [Nhel site-trpA terminator-R6K Origin-RNA-OUT-KpnI site]. The NTC9685R nanoplasmid vector comprises a R6K Replication origin, a spacer region encoded bacterial region (replication and selection) of 466 bp [Nhel site-trpA terminator-R6K Origin-RNA-OUT-KpnI site], and a VA1 RNA and SV40 enhancer.

In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to a sequence selected from the group consisting of SEQ ID NO: 2668, SEQ ID NO: 2669, SEQ ID NO: 2670, or SEQ ID NO: 2671, as set forth below. In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2668. In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2669. In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2670. In some embodiments, the nanoplasmid vector comprises a nucleotide sequence having at least 80%, 85%, 90%, 91%, 92%, 93%, 94%, 95%, 96%, 97%, 98%, 99%, or 99.5% sequence identity to the sequence of SEQ ID NO: 2671.

In some embodiments, the nanoplasmid vector comprises modifications that improve the replication of the vector. In some embodiments, the nanoplasmid vector utilizes a Pol III-dependent origin of replication to replicate. In some embodiments, the nanoplasmid vector utilizes a Pol I-dependent origin of replication to replicate. In some embodiments, the nanoplasmid vector comprises an antibiotic selectable marker. In some embodiments, the nanoplasmid vector does not comprise an antibiotic selectable marker. In some embodiments, the nanoplasmid vector comprises an RNA selectable marker.

B. Other Methods of Modification

In some embodiments of the methods of the disclosure, a modified immune cell of the disclosure may be produced by introducing a transgene into an immune cell of the disclosure. The introducing step may comprise delivery of a nucleic acid sequence and/or a genomic editing construct via a non-transposition delivery system.

In some embodiments of the methods of the disclosure, introducing a nucleic acid sequence and/or a genomic editing construct into an immune cell ex vivo, in vivo, in vitro or in situ comprises one or more of topical delivery, adsorption, absorption, electroporation, spin-fection, co-culture, transfection, mechanical delivery, sonic delivery, vibrational delivery, magnetofection or by nanoparticle-mediated delivery. In some embodiments of the methods of the disclosure, introducing a nucleic acid sequence and/or a genomic editing construct into an immune cell ex vivo, in vivo, in vitro or in situ comprises liposomal transfection, calcium phosphate transfection, fugene transfection, and dendrimer-mediated transfection. In some embodiments of the methods of the disclosure, introducing a nucleic acid sequence and/or a genomic editing construct into an immune cell ex vivo, in vivo, in vitro or in situ by mechanical transfection comprises cell squeezing, cell bombardment, or gene gun techniques. In some embodiments of the methods of the disclosure, introducing a nucleic acid sequence and/or a genomic editing construct into an immune cell ex vivo, in vivo, in vitro or in situ by nanoparticle-mediated transfection comprises liposomal delivery, delivery by micelles, and delivery by polymerosomes.

In some embodiments of the methods of the disclosure, introducing a nucleic acid sequence and/or a genomic editing construct into an immune cell ex vivo, in vivo, in vitro or in situ comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In some embodiments, the non-viral vector comprises a transposon of the disclosure.

In some embodiments of the methods of the disclosure, enzymes may be used to create strand breaks in the host genome to facilitate delivery or integration of the transgene. In some embodiments, enzymes create single-strand breaks. In some embodiments, enzymes create double-strand breaks. In some embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) or zinc finger nucleases (ZFN). Other editing or break-inducing enzymes may include, without limitation, nucleases such as Cas12a (includes MAD7), Cas12b, Cas12c, Cas13, and many more. In certain instance, the Cas12a nuclease is MAD7.

In some embodiments, break-inducing enzymes can be delivered to the cell encoded in DNA, encoded in mRNA, as a protein, as a nucleoprotein complex with a guide RNA (gRNA).

In some embodiments of the methods of the disclosure, the site-specific transgene integration is controlled by a vector-mediated integration site bias. In some embodiments vector-mediated integration site bias is controlled by the chosen lentiviral vector. In some embodiments vector-mediated integration site bias is controlled by the chosen gamma-retroviral vector.

In some embodiments of the methods of the disclosure, the site-specific transgene integration site is a non-stable chromosomal insertion. In some embodiments, the integrated transgene may become silenced, removed, excised, or further modified.

In some embodiments of the methods of the disclosure, the genome modification is a non-stable integration of a transgene. In some embodiments, the non-stable integration can be a transient non-chromosomal integration, a semi-stable non chromosomal integration, a semi-persistent non-chromosomal insertion, or a non-stable chromosomal insertion. In some embodiments, the transient non-chromosomal insertion can be epi-chromosomal or cytoplasmic.

In some embodiments, the transient non-chromosomal insertion of a transgene does not integrate into a chromosome and the modified genetic material is not replicated during cell division.

In some embodiments of the methods of the disclosure, the genome modification is a semi-stable or persistent non-chromosomal integration of a transgene. In some embodiments, a DNA vector encodes a Scaffold/matrix attachment region (S-MAR) module that binds to nuclear matrix proteins for episomal retention of a non-viral vector allowing for autonomous replication in the nucleus of dividing cells.

In some embodiments of the methods of the disclosure, the genome modification is a non-stable chromosomal integration of a transgene. In some embodiments, the integrated transgene may become silenced, removed, excised, or further modified.

In some embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In some embodiments, the modification to the genome by transgene insertion can occur via CRISPR-Cas9, TALEN or ZFNs.

C. Nanoparticle Delivery

The term “gene editing” as used herein refers to the insertion, deletion or replacement of nucleic acids in genomic DNA so as to add, disrupt or modify the function of the product that is encoded by a gene. Various gene editing systems require, at a minimum, the introduction of a cutting enzyme (e.g., a nuclease or recombinase) that cuts genomic DNA to disrupt or activate gene function.

Further, in gene editing systems that involve inserting new or existing nucleotides/nucleic acids, insertion tools (e.g., DNA template vectors, transposable elements (transposons or retrotransposons) must be delivered to the cell in addition to the cutting enzyme (e.g., a nuclease, recombinase, integrase or transposase). Examples of such insertion tools for a recombinase may include a DNA vector. Other gene editing systems require the delivery of an integrase along with an insertion vector, a transposase along with a transposon/retrotransposon, etc. In some embodiments, an example recombinase that may be used as a cutting enzyme is the CRE recombinase. In various embodiments, example integrases that may be used in insertion tools include viral based enzymes taken from any of a number of viruses including, but not limited to, AAV, gamma retrovirus, and lentivirus. Example transposons/retrotransposons that may be used in insertion tools include, but are not limited to, the piggyBac® transposon, Sleeping Beauty transposon, TcBuster transposon and the L1 retrotransposon.

In certain embodiments of the methods of the disclosure, non-viral vectors are used for transgene delivery. In certain embodiments, the non-viral vector is a nucleic acid. In certain embodiments, the nucleic acid non-viral vector is plasmid DNA, linear double-stranded DNA (dsDNA), linear single-stranded DNA (ssDNA), DoggyBone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In certain embodiments, the non-viral vector is a transposon. In certain embodiments, the transposon is TcBuster.

In certain embodiments of the methods of the disclosure, transgene delivery can occur via viral vector. In certain embodiments, the viral vector is a non-integrating non-chromosomal vectors. Non-integrating non-chromosomal vectors can include adeno-associated virus (AAV), adenovirus, and herpes viruses. In certain embodiments, the viral vector is an integrating chromosomal vectors. Integrating chromosomal vectors can include adeno-associated vectors (AAV), Lentiviruses, and gamma-retroviruses.

In certain embodiments of the methods of the disclosure, transgene delivery can occur by a combination of vectors. Exemplary but non-limiting vector combinations can include: viral plus non-viral vectors, more than one non-viral vector, or more than one viral vector. Exemplary but non-limiting vectors combinations can include: DNA-derived plus RNA-derived vectors, RNA plus reverse transcriptase, a transposon and a transposase, a non-viral vectors plus an endonuclease, and a viral vector plus an endonuclease.

In certain embodiments of the methods of the disclosure, the genome modification can be a stable integration of a transgene, a transient integration of a transgene, a site-specific integration of a transgene, or a biased integration of a transgene.

In certain embodiments of the methods of the disclosure, the genome modification can be a stable chromosomal integration of a transgene. In certain embodiments, the stable chromosomal integration can be a random integration, a site-specific integration, or a biased integration. In certain embodiments, the site-specific integration can be non-assisted or assisted. In certain embodiments, the assisted site-specific integration is co-delivered with a site-directed nuclease. In certain embodiments, the site-directed nuclease comprises a transgene with 5′ and 3′ nucleotide sequence extensions that contain homology to upstream and downstream regions of the site of genomic integration. In certain embodiments, the transgene with homologous nucleotide extensions enable genomic integration by homologous recombination, microhomology-mediated end joining, or nonhomologous end-joining. In certain embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

In certain embodiments, the site-specific transgene integration occurs at a site that disrupts expression of a target gene. In certain embodiments, disruption of target gene expression occurs by site-specific integration at introns, exons, promoters, genetic elements, enhancers, suppressors, start codons, stop codons, and response elements. In certain embodiments, exemplary target genes targeted by site-specific integration include but are not limited to CD70 or PD1, any immunosuppressive gene, and genes involved in allo-rejection.

In certain embodiments, the site-specific transgene integration occurs at a site that results in enhanced expression of a target gene. In certain embodiments, enhancement of target gene expression occurs by site-specific integration at introns, exons, promoters, genetic elements, enhancers, suppressors, start codons, stop codons, and response elements.

In certain embodiments of the methods of the disclosure, enzymes may be used to create strand breaks in the host genome to facilitate delivery or integration of the transgene. In certain embodiments, enzymes create single-strand breaks. In certain embodiments, enzymes create double-strand breaks. In certain embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) and zinc finger nucleases (ZFN). In certain embodiments, break-inducing enzymes can be delivered to the cell encoded in DNA, encoded in mRNA, as a protein, as a nucleoprotein complex with a guide RNA (gRNA).

In certain embodiments of the methods of the disclosure, the site-specific transgene integration is controlled by a vector-mediated integration site bias. In certain embodiments vector-mediated integration site bias is controlled by the chosen lentiviral vector. In certain embodiments vector-mediated integration site bias is controlled by the chosen gamma-retroviral vector.

In certain embodiments of the methods of the disclosure, the site-specific transgene integration site is a non-stable chromosomal insertion. In certain embodiments, the integrated transgene may become silenced, removed, excised, or further modified. In certain embodiments of the methods of the disclosure, the genome modification is a non-stable integration of a transgene. In certain embodiments, the non-stable integration can be a transient non-chromosomal integration, a semi-stable non chromosomal integration, a semi-persistent non-chromosomal insertion, or a non-stable chromosomal insertion. In certain embodiments, the transient non-chromosomal insertion can be epi-chromosomal or cytoplasmic. In certain embodiments, the transient non-chromosomal insertion of a transgene does not integrate into a chromosome and the modified genetic material is not replicated during cell division.

In certain embodiments of the methods of the disclosure, the genome modification is a semi-stable or persistent non-chromosomal integration of a transgene. In certain embodiments, a DNA vector encodes a Scaffold/matrix attachment region (S-MAR) module that binds to nuclear matrix proteins for episomal retention of a non-viral vector allowing for autonomous replication in the nucleus of dividing cells.

In certain embodiments of the methods of the disclosure, the genome modification is a non-stable chromosomal integration of a transgene. In certain embodiments, the integrated transgene may become silenced, removed, excised, or further modified.

In certain embodiments of the methods of the disclosure, the modification to the genome by transgene insertion can occur via host cell-directed double-strand breakage repair (homology-directed repair) by homologous recombination (HR), microhomology-mediated end joining (MMEJ), nonhomologous end joining (NHEJ), transposase enzyme-mediated modification, integrase enzyme-mediated modification, endonuclease enzyme-mediated modification, or recombinant enzyme-mediated modification. In certain embodiments, the modification to the genome by transgene insertion can occur via CRISPR-Cas9, CRISPR-CasX, TALEN or ZFNs.

In certain embodiments of the methods of the disclosure, a cell with an in vivo or ex vivo genomic modification can be a germline cell or a somatic cell. In certain embodiments the genetically engineered cell can be a human, non-human, mammalian, rat, mouse, or dog cell. In certain embodiments, the genetically engineered cell can be differentiated, undifferentiated, or immortalized. In certain embodiments, the genetically engineered undifferentiated cell can be a stem cell. In certain embodiments, the genetically engineered cell can be differentiated, undifferentiated, or immortalized. In certain embodiments, the genetically engineered undifferentiated cell can be an induced pluripotent stem cell. In certain embodiments, the genetically engineered cell can be a T cell, a hematopoietic stem cell, a natural killer cell, a macrophage, a dendritic cell, a monocyte, a megakaryocyte, or an osteoclast. In certain embodiments, the genetically engineered cell can be modified while the cell is quiescent, in an activated state, resting, in interphase, in prophase, in metaphase, in anaphase, or in telophase. In certain embodiments, the genetically engineered cell can be fresh, cryopreserved, bulk, sorted into sub-populations, from whole blood, from leukapheresis, or from an immortalized cell line.

D. Click Chemistry

Engineered immune cells (e.g., NK cells) described herein can also be produced using coupling reagents to link an exogenous polypeptide (cytokine, targeting moiety etc.) to a cell with the use of click chemistry reactions. Coupling reagents can be used to couple an exogenous polypeptide to a cell, for example, when the exogenous polypeptide is a complex or difficult to express polypeptide, e.g., a polypeptide, e.g., a multimeric polypeptide; large polypeptide; polypeptide derivatized in vitro; an exogenous polypeptide that may have toxicity to, or which is not expressed efficiently in, the NK cells.

The click chemistry approach was originally conceived as a method to rapidly generate complex substances by joining small subunits together in a modular fashion. (See, e.g., Kolb et al., Angew Chem. Int. Ed. 40:3004-31, 2004; Evans, Aust. J. Chem. 60:384-95, 2007.) Various forms of click chemistry reaction are known in the art, such as the Huisgen 1,3-dipolar cycloaddition copper catalyzed reaction (Tornoe et al., J. Organic Chem. 67:3057-64, 2002), which is often referred to as the “click reaction.” Other alternatives include cycloaddition reactions such as the Diels-Alder, nucleophilic substitution reactions (especially to small strained rings like epoxy and aziridine compounds), carbonyl chemistry formation of urea compounds and reactions involving carbon-carbon double bonds, such as alkynes in thiol-yne reactions. In some embodiments, the click chemistry approach comprises copper catalyzed reaction, as described, e.g., in Rostovstev et al. Angew Chem Int Ed 41:2596, 2002; Tomoe et al. J. Org. Chem. 67:3057, 2002. In other embodiments, the click chemistry approach comprises copper-free click reaction, as described, e.g., by Agard et al. J. Am. Chem. Soc. 126:15046-47, 2004, and Ning et al. Angew Chem. Int. Ed. 49:3065-68, 2010.

E. Sortases

In some embodiments, an exogenous polypeptide described herein can be conjugated to the surface of an immune cell (e.g., an NK cell) by various chemical and enzymatic means, including but not limited to chemical conjugation with bifunctional cross-linking agents such as, e.g., an NHS ester-maleimide heterobifunctional crosslinker to connect a primary amine group with a reduced thiol group. These methods also include enzymatic strategies such as, e.g., transpeptidase reaction mediated by a sortase enzyme.

Sortase transpeptidation, also known as “sortase labeling” or “sortagging,” can be used for bioconjugation of two proteins. Methods and compositions disclosed herein can use or include a sortase from any bacterial species or strain, e.g., a sortase A, a sortase B, a sortase C, a sortase D, a sortase E, a sortase F, or a sortase from a yet unidentified class of sortase enzymes. All gram-positive bacteria examined to date possess at least one major housekeeping sortase (e.g., sortase A) (Barnett et al., J. Bacteriol. 186(17):5865-75, 2004). The methods described herein can be used to evaluate candidate sortases. The amino acid sequences of many sortases and the nucleotide sequences that encode them are known to those of skill in the art and are disclosed in many of the references cited herein. The amino acid sequence of full-length, wild-type S. aureus sortase A comprises the amino acid sequence of SEQ ID NO: 683. Wild-type and mutant sortase molecules can be used to form CAR members, e.g., in situ on immune effector cells that comprise a sortase acceptor motif. An exemplary sortase mutant, which is efficient, and not dependent on non-physiological reaction conditions, is S. aureus Sortase A mutant [P94R/E105K/E108Q/D160N/D165 Å/K190E/K196T]. This mutant lacks the N-terminal 59 amino acid residues of S. aureus sortase A and includes amino acid substitutions that render the enzyme calcium-independent and which make the enzyme faster (amino acid residue numbers herein begin with residue the first residue at the N-terminal end of non-truncated S. aureus sortase A). The primary amino acid sequence of Sortase A mutant [P94R/E105K/E108Q/D160N/D165 Å/K190E/K196T] comprises the amino acid sequence of SEQ ID NO: 684.

In some embodiments, the sortase recognition motif is LPXTG (SEQ ID NO: 685) or LPXTA (SEQ ID NO: 686) and the sortase acceptor motif is N-terminal donor sequence GGG, resulting in the sortase transfer signature that comprises LPXTGG (SEQ ID NO: 5) after sortase-mediated reaction (Swee et al. Proc. Nat'l. Acad. Sci. USA 110(4):1428-33, 2013). The methods also include combination methods, such as e.g., sortase-mediated conjugation of Click Chemistry handles or “click handles” (an azide and an alkyne) on the antigen and the cell, respectively, followed by a cyclo-addition reaction to chemically bond a polypeptide to a cell, see e.g., Neves et al. Bioconjug. Chem. 24(6): 934-41, 2013. Sortase-mediated modification of proteins is described in WO 2014/183066, WO 2014/183071, and WO 2016/014553 each of which are incorporated by reference in their entireties herein.

In some embodiments, a protein is modified by the conjugation of a sortase substrate comprising an amino acid, a peptide, a protein, a polynucleotide, a carbohydrate, a tag, a metal atom, a contrast agent, a catalyst, a non-polypeptide polymer, a recognition element, a small molecule, a lipid, a linker, a label, an epitope, an antigen, a therapeutic agent, a toxin, a radioisotope, a particle, or moiety comprising a reactive chemical group, e.g., a click chemistry handle.

If desired, a catalytic bond-forming polypeptide domain can be expressed on an NK cell extracellularly. Many catalytic bond-forming polypeptides exist, including transpeptidases, sortases, and isopeptidases, including those derived from Spy0128, a protein isolated from Streptococcus pyogenes.

It has been demonstrated that splitting the autocatalytic isopeptide bond-forming subunit (CnaB2 domain) of Spy0128 results in two distinct polypeptides that retain catalytic activity with specificity for each other. The polypeptides in this system are termed SpyTag and SpyCatcher. Upon mixing, SpyTag and SpyCatcher undergo isopeptide bond formation between Asp117 on SpyTag and Lys31 on SpyCatcher (Zakeri and Howarth, J. Am. Chem. Soc. 132:4526, 2010). The reaction is compatible with the cellular environment and highly specific for protein/peptide conjugation (Zakeri et al., Proc. Natl. Acad. Sci. U.S.A. 109:E690-E697, 2012). SpyTag and SpyCatcher has been shown to direct post-translational topological modification in elastin-like protein. For example, placement of SpyTag at the N-terminus and SpyCatcher at the C-terminus directs formation of circular elastin-like proteins (Zhang et al. J. Am. Chem. Soc. 135(37):13988-97, 2013).

The components SpyTag and SpyCatcher can be interchanged such that a system in which molecule A is fused to SpyTag and molecule B is fused to SpyCatcher is functionally equivalent to a system in which molecule A is fused to SpyCatcher and molecule B is fused to SpyTag. For the purposes of this document, when SpyTag and SpyCatcher are used, it is to be understood that the complementary molecule could be substituted in its place.

A catalytic bond-forming polypeptide, such as a SpyTag/SpyCatcher system, can be used to attach the exogenous polypeptide to the surface of an NK cell to make an engineered NK cell. The SpyTag polypeptide sequence can be expressed on the extracellular surface of the NK cell. The SpyTag polypeptide can be, for example, fused to the N terminus of a transmembrane protein, e.g., inserted in-frame at the extracellular terminus or in an extracellular loop of a multi-pass transmembrane protein, fused to a lipid-chain-anchored polypeptide, or fused to a peripheral membrane protein. The nucleic acid sequence encoding the SpyTag fusion can be expressed within an engineered NK cell. An exogenous stimulatory polypeptide can be fused to SpyCatcher. The nucleic acid sequence encoding the SpyCatcher fusion can be expressed and secreted from the same NK cell that expresses the SpyTag fusion. Alternatively, the nucleic acid sequence encoding the SpyCatcher fusion can be produced exogenously, for example in a bacterial, fungal, insect, mammalian, or cell-free production system. Upon reaction of the SpyTag and SpyCatcher polypeptides, a covalent bond will be formed that attaches the exogenous stimulatory polypeptide to the surface of the NK cell to form an engineered NK cell.

F. Methods of NK Cell Expansion

Provided herein are methods of making a population of genetically engineered NK cells that include contacting a population of NK cells (e.g., any of the NK cell populations described herein) with a CD70 inhibitor (e.g., any of the exemplary CD70 inhibitors described herein), and expanding the population of NK cells in vitro (e.g., using any of the exemplary techniques described herein). In some embodiments, a CD70 inhibitor is a small interfering RNA (siRNA) that targets CD70 mRNA, a short hairpin RNA (shRNA) that targets CD70 mRNA, a nucleic acid encoding a siRNA that targets CD70 mRNA, a nucleic acid encoding an shRNA that targets CD70 mRNA, or a combination of any of the foregoing. In some embodiments, the CD70 inhibitor comprises an RNA-guided endonuclease and a guide RNA (gRNA) targeting a CD70 gene. In some embodiments, the CD70 inhibitor decreases cell surface level of CD70 polypeptide in at least one NK cell of the population of NK cells. In some embodiments, the CD70 inhibitor comprises a Protein Expression Blocker (PEBL) or a nucleic acid encoding a PEBL, wherein the PEBL comprises a first antigen recognition domain that specifically binds human CD70 and one or more of a localizing domain, an intracellular retention domain and an endoplasmic reticulum (ER) retention domain. In some embodiments, the CD70 inhibitor comprises an antagonistic anti-CD70 antibody or an antigen-binding fragment thereof.

Following genetic modification the cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the chimeric receptor is expanded ex vivo. In some embodiments, the clone is expanded at least 1,000-fold in culture. In certain embodiments, the NK cells (e.g., NK cell clones) are expanded in culture by about 1-1000 fold, such as by about 1-950 fold, 1-900 fold, 1-850 fold, 1-800 fold, 1-750 fold, 1-700 fold, 1-650 fold, 1-600 fold, 1-550 fold, 1-500 fold, 1-450 fold, 1-400 fold, 1-350 fold, 1-300 fold, 1-250 fold, 1-200 fold, 1-150 fold, 1-100 fold, 1-50 fold, 1-10 fold, 10-1000 fold, 10-950 fold, 10-900 fold, 10-800 fold, 10-700 fold, 10-600 fold, 10-500 fold, 10-400 fold, 10-300 fold, 10-200 fold, 10-100 fold, 10-50 fold, 20-1000 fold, 20-900 fold, 20-800 fold, 20-700 fold, 20-600 fold, 20-500 fold, 20-400 fold, 20-300 fold, 20-200 fold, 20-100 fold, 20-50 fold, 30-1000 fold, 30-900 fold, 30-800 fold, 30-700 fold, 30-600 fold, 30-500 fold, 30-400 fold, 30-300 fold, 30-200 fold, 30-100 fold, 30-50 fold, 40-1000 fold, 40-900 fold, 40-800 fold, 40-700 fold, 40-600 fold, 40-500 fold, 40-400 fold, 40-300 fold, 40-200 fold, 40-100 fold, 40-50 fold, 50-1000 fold, 50-900 fold, 50-800 fold, 50-700 fold, 50-600 fold, 50-500 fold, 50-400 fold, 50-300 fold, 50-200 fold, 50-100 fold, 100-1000 fold, 100-900 fold, 100-800 fold, 100-700 fold, 100-600 fold, 100-500 fold, 100-400 fold, 100-300 fold, 100-200 fold, 200-1000 fold, 200-900 fold, 200-800 fold, 200-700 fold, 200-600 fold, 200-500 fold, 200-400 fold, 200-300 fold, 300-1000 fold, 300-900 fold, 300-800 fold, 300-700 fold, 300-600 fold, 300-500 fold, 300-400 fold, 400-1000 fold, 400-900 fold, 400-800 fold, 400-700 fold, 400-600 fold, 400-500 fold, 500-1000 fold, 500-900 fold, 500-800 fold, 500-700 fold, or 500-600 fold. In some embodiments, the cells are expanded in the absence of feeder cells. The clone selected for expansion demonstrates the capacity to specifically recognize and lyse CD70 expressing target cells. The recombinant immune cells may be expanded by stimulation with IL-2, or other cytokines that bind the common gamma-chain (e.g., IL-7, IL-12, IL-15, IL-21, and others). The recombinant NK cells may be expanded by stimulation with artificial antigen presenting cells. In a further aspect, the genetically engineered cells may be cryopreserved.

### 3. Modification of Gene and Polypeptide Expression

In some embodiments, the NK cells or populations of NK cells of the present disclosure are modified to have altered expression of cellular genes and/or polypeptides, such as CD70, glucocorticoid receptor, TGF beta receptor (e.g., TGFBR1 or TGFBR2), PD1, and/or CISH. In some embodiments an altered expression is a decreased expression of gene and/or polypeptide in at least one NK cell of a population of cells. In some embodiments an altered expression refers to a knockout of the gene. In some embodiments, an altered expression refers to a knockdown of the gene. In some embodiments, an altered expression refers to a reduced expression and/or levels of a polypeptide. In some embodiments, an altered expression refers to an ablation of polypeptide expression. In some embodiments, altered expression refers to sequestration of the polypeptide to internal compartments of the cell and/or a decreased expression or levels of surface polypeptides.

In some embodiments, the NK cells of the present disclosure are contacted with a CD70 inhibitor and modified to have an altered gene and/or polypeptide expression of CD70. Thus, this disclosure provides methods of making a population of genetically engineered NK cells by (a) providing a population of NK cells, contacting the population of NK cells with a CD70 inhibitor; and (c) expanding the population of NK cells in vitro.

In some embodiments, the NK cells of the present disclosure are modified to have reduced expression and/or levels of CD70. In some embodiments, the NK cells have been genetically engineered to disrupt expression of endogenous CD70. In some embodiments, an NK cells have been genetically engineered to disrupt expression and/or levels of endogenous CD70 on the cell surface. In some embodiments, disruption of e expression and/or levels of endogenous CD70 on the cell surface is achieved by sequestration of endogenous CD70 to an intracellular compartment(s).

In some embodiments, an NK cell is contacted with a CD70 inhibitor that disrupts expression of endogenous CD70. This disclosure provides a method of making a population of genetically engineered natural killer (NK) cells, the method comprising (a) providing a population of NK cells; (b) contacting the population of NK cells with a CD70 inhibitor; and (c) expanding the population of NK cells in vitro.

In some embodiments, (b) contacting the population of NK cells with a CD70 inhibitor may occur prior to (c) expanding the population of NK cells in vitro. In some embodiments, (b) contacting the population of NK cells with a CD70 inhibitor may occur after (c) expanding the population of NK cells in vitro. In some embodiments, (b) contacting the population of NK cells with a CD70 inhibitor may occur concurrently with (c) expanding the population of NK cells in vitro. In some embodiments, (b) contacting the population of NK cells with a CD70 inhibitor may occur prior to, concurrently, and/or after (c) expanding the population of NK cells in vitro.

In some embodiments, the population of NK cells is contacted with a CD70 inhibitor for at least about 1 day, at least about 2 days, at least about 3 days, at least about 4 days, at least about 5 days, at least about 6 days, or at least about 7 days. In some embodiments, the population of NK cells is contacted with a CD70 inhibitor for at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours.

In some embodiments, following the contacting with a CD70 inhibitor, the population of NK cells is depleted of any CD70⁺ NK cells. For example, CD70⁺ NK cells may be depleted using methods known in the art including depletion with anti-CD70 antibody-coated magnetic beads.

Also provided herein is a genetically engineered natural killer (NK) cell modified to have a) a decreased level of total CD70 polypeptide as compared to the level of total CD70 polypeptide in a wild-type NK cell, and/or b) a decreased level of CD70 polypeptide on the cell surface as compared to the level of CD70 on the cell surface in a wild-type NK cell.

In some embodiments, the genetically engineered NK has a reduced likelihood of fratricide by a NK cell expressing an anti-CD70 CAR compared to the likelihood of fratricide of a NK cell that has not been modified to one or more of: (a) a decreased level of CD70 polypeptide compared to the level of total CD70 polypeptide in a wild-type NK cell; (b) a decreased level of CD70 polypeptide on the cell surface as compared to the level of CD70 on the cell surface in a wild-type NK cell (c) a decreased level of total CD70 polypeptide as compared to the level of total CD70 polypeptide in a wild-type NK cell comprising an anti-CD70 CAR; and (d) a decreased level of CD70 polypeptide on the cell surface as compared to the level of CD70 on the cell surface in a wild-type NK cell comprising an anti-CD70 CAR.

In some embodiments, the genetically engineered NK cell exhibits greater cell expansion rate than a NK cell that has not been modified to one or more of: (a) a decreased level of total CD70 polypeptide as compared to the level of total CD70 polypeptide in a wild-type NK cell; (b) a decreased level of CD70 polypeptide on the cell surface as compared to the level of CD70 on the cell surface in a wild-type NK cell (c) a decreased level of total CD70 polypeptide as compared to the level of total CD70 polypeptide in a wild-type NK cell comprising an anti-CD70 CAR; and (d) a decreased level of CD70 polypeptide on the cell surface as compared to the level of CD70 on the cell surface in a wild-type NK cell comprising an anti-CD70 CAR.

In some embodiments, the genetically engineered NK cell comprises a disrupted CD70 gene. In some embodiments, the genetically engineered NK cell comprises a knockout or knockdown of a CD70 gene. In some embodiments, the genetically engineered NK cell comprises at least about 10% less, about 20% less, about 30% less, about 40% less, about 50% less, about 60% less, about 70% less, about 80% less, or about 90% less of CD70 polypeptide on the cell surface and/or total CD70 polypeptide than the wild-type NK cell.

In some embodiments, the level of CD70 mRNA in the NK cell is reduced and wherein the level of CD70 mRNA is measured by Northern blot, quantitative PCR, or RNA sequencing. In some embodiments, the level of CD70 polypeptide in the NK cell is reduced and wherein the level of CD70 polypeptide is measured by Western blot, ELISA, flow cytometry, or mass spectrometry.

Further provided herein is a population of NK cells, wherein at least about 30%, about 40%, about 50%, about 60%, about 70%, about 80%, about 90% or about 95% of the cells in the population are the genetically engineered NK cells disclosed herein (e.g., comprising one or more polypeptides and/or nucleic acids described herein). Further provided herein is a pharmaceutical composition comprising any of the genetically engineered NK cells disclosed herein or a population of any of the genetically engineered NK cells disclosed herein, and a pharmaceutically acceptable carrier, diluent or excipient.

A. CD70 Inhibitors

Anti-CD70 Antibodies

In some embodiments, a CD70 inhibitor is an antagonistic anti-CD70 antibody or an antigen-binding fragment thereof. In some embodiments, the antagonistic anti-CD70 antibody binds to CD70 but does not induce signal transduction. In some embodiments, the antagonistic anti-CD70 antibody inhibits the interaction between CD70 and CD27. Methods of determining whether an antibody inhibits the interaction between CD70 and CD27 are known in the art (e.g., ELISA). Exemplary antagonistic anti-CD70 antibodies include but are not limited to cusatuzumab (ARGX-110), MDX-1411, SGN70, 27B3, 57B6, 59D10, 19G10, 9B2, 5B2, 9G2, 5F4, and 9D1. Other exemplary antagonistic anti-CD70 antibodies are described in U.S. Pat. No. 9,765,148 (incorporated herein by reference).

siRNA and shRNA Targeting CD70 mRNA Expression

In some embodiments, the CD70 inhibitor comprises a small interfering RNA (siRNA) that targets CD70 mRNA, a short hairpin RNA (shRNA) that targets CD70 mRNA, a nucleic acid encoding a siRNA that targets CD70 mRNA, a nucleic acid encoding an shRNA that targets CD70 mRNA, or a combination of any of the foregoing. In some embodiments, the genetically engineered NK cell comprises an siRNA that targets CD70 mRNA and/or an shRNA that targets CD70 mRNA disclosed herein. In some embodiments, the genetically engineered NK cell comprises a nucleic acid sequence encoding an siRNA that targets CD70 mRNA and/or an shRNA that targets CD70 mRNA disclosed herein.

In some embodiments, the NK cells of the present disclosure are further modified to have altered expression of other cellular genes and/or polypeptides. For example, cytokine signaling is essential for the normal function of hematopoietic cells. The SOCS family of proteins plays an important role in the negative regulation of cytokine signaling, acting as an intrinsic brake. CIS, a member of the SOCS family of proteins encoded by the CISH gene, has been identified as an important checkpoint molecule in NK cells in mice. In some embodiments, SOCS family proteins encoded by the CISH gene are knocked out in immune cells to improve cytotoxicity, such as in NK cells. Exemplary SOCS family of proteins include, but are not limited to SOCS1, SOCS2, SOCS3 and CISH. This approach may be used alone or in combination with other checkpoint inhibitors to improve anti-tumor activity.

In some embodiments, the altered gene expression is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the altered gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the alteration of the expression, activity, and/or function of the gene is carried out by disrupting the gene. In some aspects, the gene is modified so that its expression is reduced by at least at or about 20, 30, or 40%, generally at least at or about 50, 60, 70, 80, 90, or 95% as compared to the expression in the absence of the gene modification or in the absence of the components introduced to effect the modification.

In some embodiments, the alteration is transient or reversible, such that expression of the gene is restored at a later time. In other embodiments, the alteration is not reversible or transient, e.g., is permanent.

In some embodiments, gene alteration is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, alteration in gene expression is achieved using antisense techniques, such as by RNA interference (RNAi), short interfering RNA (siRNA), short hairpin (shRNA), tandem shRNA, and/or ribozymes to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct. siRNA and shRNA may be delivered into a cell using any method known in the art, including via transfection, liposomes, chemical solvents, electroporation, viral vectors, pinocytosis, phagocytosis and other forms of spontaneous or induced cellular uptake. For example, transfection reagents that may be used to deliver an siRNA or shRNA of the disclosure to a cell include, but are not limited to, DharmaFECT 1, DharmaFECT 2, DharmaFECT 3, DharmaFECT 4, Lipofectamine 2000, Lipfectamine 3000, or Lipofectamine RNAiMAX.

Inhibitory molecules, (e.g., PD1 or TGFbeta receptor) can, in some instances, decrease the ability of an immune cell (e.g. an NK cell) to mount an immune effector response. Inhibition of an inhibitory molecule, e.g., by inhibition at the DNA, RNA or protein level, can optimize the immune cell performance. In some embodiments, an inhibitory nucleic acid, e.g., an inhibitory nucleic acid, e.g., a dsRNA, e.g., an siRNA or shRNA, can be used to inhibit expression of an inhibitory molecule in the NK cell. In some embodiments, the inhibitory nucleic acid is a shRNA. In some embodiments, the inhibitory molecule is inhibited within a NK cell. In these instances, a dsRNA molecule that inhibits expression of the inhibitory molecule is linked to the nucleic acid that encodes a component, e.g., all of the components, of the CAR. Examples of inhibitory molecules include but are not limited to SOCS, CISH, PD1 and TGFbeta receptor (TGFBR).

In some embodiments, a CD70 inhibitor decreases the expression and/or levels of CD70 polypeptide in cells. In some embodiments, expression of the CD70 polypeptide is ablated. Exemplary CD70 inhibitors may include but are not limited to an siRNA, an shRNA, a dsRNA or any combination thereof that targets a CD70 mRNA.

The gene expression modification techniques above can be used to disrupt the expression of a protein, for example CD70, on NK cells of the disclosure. The cells with a disrupted CD70 gene retain CAR NK cell function even where fratricide may be expected. Cells with CD70 gene expression modification (e.g., in which the CD70 gene has been disrupted using gene editing technology), independent of the CAR insertion, exhibit continued, steady cell growth, relative to unmodified NK cells (or edited NK cells that express CD70). In some embodiments, a disrupted gene is a gene that does not encode functional protein. In some embodiments, a cell that comprises a disrupted gene does not express or have (e.g., at the cell surface) a detectable level (e.g. by antibody, e.g., by flow cytometry) of the protein encoded by the gene. A cell that does not express or have a detectable level of the protein may be referred to as a knockout cell. For example, a cell having a CD70 gene expression modification may be considered a CD70 knockout cell if CD70 protein cannot be detected at the cell surface using an antibody that specifically binds CD70 protein. Exemplary shRNA construct sequences that may be used to disrupt the expression of CD70 on NK cells of the disclosure are provided in Tables 11 and 12.

TABLE 11
Exemplary shRNA Constructs targeting CD70

| | | |
|---|---|---|
| Exemplary | | |
| Construct | | SEQ ID |
| Components | Nucleic Acid Sequences | NO: |

shRNA

| | | |
|---|---|---|
| CD70-shRNA1 | GAAACACTGATGAGACCTT | 2647 |
| CD70-shRNA2 | CCATCGTGATGGCATCTACAT | 2648 |
| CD70-shRNA3 | GTAGCTGAGCTGCAGCTGAAT | 2649 |
| CD70-shRNA4 | TGGCATCTACATGGTACACAT | 2650 |
| CD70-shRNA5 | CAGCTACGTATCCATCGTGAT | 2651 |
| CD70-shRNA6 | ACACACTCTGCACCAACCTCA | 2652 |

shRNA elements

| | | |
|---|---|---|
| U6 Promoter | GAGGGCCTATTTCCCATGATTCCTTCATATTTGCATATACGATACAAGGC | 2653 |
| | TGTTAGAGAGATAATTGGAATTAATTTGACTGTAAACACAAAGATATTAG | |
| | TACAAAATACGTGACGTAGAAAGTAATAATTTCTTGGGTAGTTTGCAGTT | |
| | TTAAAATTATGTTTTAAAATGGACTATCATATGCTTACCGTAACTTGAAA | |
| | GTATTTCGATTTCTTGGCTTTATATATCTTGTGGAAAGGACGAAACACCG | |
| | G | |
| Loop | CTCGAG | 2654 |
| shRNA terminator | TTTTT | 2655 |

TABLE 12
Exemplary shRNA constructs regulated by U6 promoter

| Exemplary | | |
|---|---|---|
| shRNA | | |
| constructs and | | SEQ |
| Domains | Nucleic Acid Sequence | ID NO: |
| U6p-shRNA1 | GAGGGCCTATTTCCCATGATTCCTTCATATTTGCATATACGATACAAGGCTGT | 2656 |
| U6 promoter, | TAGAGAGATAATTGGAATTAATTTGACTGTAAACACAAAGATATTAGTACAAA | |
| shRNA, Loop, | ATACGTGACGTAGAAAGTAATAATTTCTTGGGTAGTTTGCAGTTTTAAAATTA | |
| shRNA, shRNA | TGTTTTAAAATGGACTATCATATGCTTACCGTAACTTGAAAGTATTTCGATTT | |
| terminator | CTTGGCTTTATATATCTTGTGGAAAGGACGAAACACCGGGAAACACTGATGAG | |
| | ACCTTCTCGAG [symbol] TTTTT | |
| U6p-shRNA2 | GAGGGCCTATTTCCCATGATTCCTTCATATTTGCATATACGATACAAGGCTGT | 2657 |
| U6 promoter, | TAGAGAGATAATTGGAATTAATTTGACTGTAAACACAAAGATATTAGTACAAA | |
| shRNA, Loop, | ATACGTGACGTAGAAAGTAATAATTTCTTGGGTAGTTTGCAGTTTTAAAATTA | |
| shRNA, shRNA | TGTTTTAAAATGGACTATCATATGCTTACCGTAACTTGAAAGTATTTCGATTT | |
| terminator | CTTGGCTTTATATATCTTGTGGAAAGGACGAAACACCGGCCATCGTGATGGCA | |
| | TCTACATCTCGAG [symbol] TTTTT | |
| U6p-shRNA3 | GAGGGCCTATTTCCCATGATTCCTTCATATTTGCATATACGATACAAGGCTGT | 2658 |
| U6 promoter, | TAGAGAGATAATTGGAATTAATTTGACTGTAAACACAAAGATATTAGTACAAA | |
| shRNA, Loop, | ATACGTGACGTAGAAAGTAATAATTTCTTGGGTAGTTTGCAGTTTTAAAATTA | |
| shRNA, shRNA | TGTTTTAAAATGGACTATCATATGCTTACCGTAACTTGAAAGTATTTCGATTT | |
| terminator | CTTGGCTTTATATATCTTGTGGAAAGGACGAAACACCGGGTAGCTGAGCTGCA | |
| | GCTGAATCTCGAG [symbol] TTTTT | |
| U6p-shRNA4 | GAGGGCCTATTTCCCATGATTCCTTCATATTTGCATATACGATACAAGGCTGT | 2659 |
| U6 promoter, | TAGAGAGATAATTGGAATTAATTTGACTGTAAACACAAAGATATTAGTACAAA | |
| shRNA, Loop, | ATACGTGACGTAGAAAGTAATAATTTCTTGGGTAGTTTGCAGTTTTAAAATTA | |
| shRNA, shRNA | TGTTTTAAAATGGACTATCATATGCTTACCGTAACTTGAAAGTATTTCGATTT | |
| terminator | CTTGGCTTTATATATCTTGTGGAAAGGACGAAACACCGGTGGCATCTACATGG | |
| | TACACATCTCGAG [symbol] TTTTT | |
| U6p-shRNA5 | GAGGGCCTATTTCCCATGATTCCTTCATATTTGCATATACGATACAAGGCTGT | 2660 |
| U6 promoter, | TAGAGAGATAATTGGAATTAATTTGACTGTAAACACAAAGATATTAGTACAAA | |
| shRNA, Loop, | ATACGTGACGTAGAAAGTAATAATTTCTTGGGTAGTTTGCAGTTTTAAAATTA | |
| shRNA, shRNA | TGTTTTAAAATGGACTATCATATGCTTACCGTAACTTGAAAGTATTTCGATTT | |
| terminator | CTTGGCTTTATATATCTTGTGGAAAGGACGAAACACCGGCAGCTACGTATCCA | |
| | TCGTGATCTCGAG [symbol] TTTTT | |
| U6p-shRNA6 | GAGGGCCTATTTCCCATGATTCCTTCATATTTGCATATACGATACAAGGCTGT | 2661 |
| U6 promoter, | TAGAGAGATAATTGGAATTAATTTGACTGTAAACACAAAGATATTAGTACAAA | |
| shRNA, Loop, | ATACGTGACGTAGAAAGTAATAATTTCTTGGGTAGTTTGCAGTTTTAAAATTA | |
| shRNA, shRNA | TGTTTTAAAATGGACTATCATATGCTTACCGTAACTTGAAAGTATTTCGATTT | |
| terminator | CTTGGCTTTATATATCTTGTGGAAAGGACGAAACACCGGACACACTCTGCACC | |
| | AACCTCACTCGAG [symbol] TTTTTT | |

Exemplary siRNA construct sequences that may be used to disrupt and/or decrease the expression and/or levels of CD70 on NK cells of the disclosure are provided in Table 13.

TABLE 13
Exemplary anti-CD70 siRNA construct sequences

| anti-CD70 | | SEQ |
|---|---|---|
| siRNA constructs | Nucleic Acid Sequence | ID NO: |
| CD70-siRNA1 | CACCAAGGUUGUACCAUUG | 2678 |
| CD70-siRNA2 | GCAUCUACAUGGUACACAU | 2679 |
| CD70-siRNA3 | GCAGCUGAAUCACACAGGA | 2680 |
| CD70-siRNA4 | UGACCACUGCUGCUGAUUA | 2681 |

Protein Expression Blocker Elements

In some embodiments, the CD70 inhibitor comprises a Protein Expression Blocker (PEBL) element. In some embodiments, the genetically engineered NK cell comprises a PEBL (e.g., a PEBL that specifically targets CD70) or a nucleic acid encoding a PEBL disclosed herein. In some embodiments, the PEBL comprises a first antigen recognition domain that specifically binds human CD70 and one or more of a localizing domain disclosed herein, an intracellular retention domain disclosed herein and an ER retention domain disclosed herein.

The present disclosure provides a population of NK cells engineered to express a chimeric antigen receptor (CAR), wherein the CAR comprises a) an antigen recognition domain, b) a hinge domain, c) a transmembrane domain, c) a costimulatory domain and e) an activation domain, and further engineered to express one or more PEBL elements.

In some embodiments, the population of NK cells expressing a CAR are further engineered to express a polypeptide construct containing a target-binding molecule that binds a target (e.g., protein) that can be removed, neutralized, or blocked from reaching the cell surface. A polypeptide comprising a antigen recognition domain linked to an intracellular localizing domain is referred to herein as “Protein Expression Blocker element” or “PEBL element” (see, e.g., WO 2018/098306 and WO 2016/126213, each of which is incorporated by reference in its entirety). In some embodiments the PEBL comprises an antigen recognition domain that specifically binds human CD70 and or more of a localizing domains, an intracellular retention domain and an endoplasmic reticulum retention domain. The antigen recognition domain is linked to a domain (e.g., a localizing domain or intracellular retention domain or endoplasmic reticulum (ER) retention domain) such that the PEBL element sequesters the target protein to specific cellular compartments, such as the golgi, endoplasmic reticulum, proteasome, or cellular membrane. In some embodiments, the PEBL element does not disrupt DNA, transcription, or translation of the target protein. In some embodiments, the PEBL element sequesters the target protein in the endoplasmic recticulum or golgi and thereby reduces the expression levels (e.g., cell surface expression levels) of the target protein. Exemplary PEBL element structures are described in Kamiya et al. (2018) Blood Adv. 2(5): 517-28.

In some embodiments, the PEBL element comprises an ER-retention domain 1 comprises an amino acid sequence having at least 90%, 91%, 92%, 93%, 94%, 95%, 96%, 97%, 98%, 99% or 100% identity with the amino acid sequence of SEQ ID NO: 2643.

In some embodiments, the PEBL element comprises an ER-retention domain 2 comprises an amino acid sequence having at least 90%, 91%, 92%, 93%, 94%, 95%, 96%, 97%, 98%, 99% or 100% identity with the amino acid sequence of SEQ ID NO: 2644.

In some embodiments, the antigen recognition domain of the PEBL element specifically bind to cell surface proteins or secreted proteins of NK cells. Exemplary target molecules include but are not limited to CD70, CS1 (SLAMF7), CD38, CD96, CTLA4, glucocorticoid receptor, TGF beta receptor (e.g., TGFbetaRII), and PD1.

In some embodiments, the antigen recognition domain of the PEBL element comprises an antibody or an antigen-binding fragment thereof of the disclosure. In some embodiments, the antigen recognition domain of the PEBL binds to CD70. In some embodiments, the antigen recognition domain comprises a CD27 polypeptide sequence or a portion thereof. In some embodiments, the antigen recognition domain of the PEBL element (e.g., anti-CD70 PEBL) is the same as the antigen recognition domain of a CAR described herein. In some embodiments, the antigen recognition domain of the PEBL element is different than the antigen recognition domain of the CAR expressed by the NK cell or population of NK cells. In some embodiments, the antigen recognition domain of the PEBL element is the same as the antigen recognition domain of the CAR expressed by the NK cell or population of NK cells.

In embodiments, the antigen recognition domain comprises a single chain antibody fragment (scFv) comprising a light chain variable domain (VL) and heavy chain variable domain (VH) of a target antigen specific monoclonal anti-CD70 antibody. Optionally, the VH and VL are joined by a flexible linker, such as a glycine-serine linker or a Whitlow linker. In embodiments, the scFv is humanized. In some embodiments, the antigen binding moiety may comprise VH and VL that are directionally linked, for example, from N to C terminus, VH-linker-VL or VL-linker-VH.

In some embodiments, the PEBL element further comprises a signal peptide domain of the disclosure. In some embodiments, the PEBL element further comprises a hinge domain of the disclosure. In some embodiments, the PEBL element comprises a transmembrane domain of the disclosure. In some embodiments, the PEBL element further comprises an activation domain of the disclosure.

In some embodiments, a CAR and a PEBL element are each encoded by a separate vector. In some embodiments the CAR is an anti-CD70 CAR. In some embodiments, the PEBL element targets CD70.

In some embodiments a CAR and a cytokine are encoded by the same vector. In some embodiments, the CAR and the PEBL element are separated by a 2A sequence. In some embodiments, the 2A sequence is a T2A sequence. In some embodiments, the 2A sequence is a P2A sequence. In some embodiments, the CAR is an anti-CD70 CAR. In some embodiments, the PEBL element targets CD70.

Table 14 shows exemplary sequences of PEBL element constructs disclosed herein comprising an anti-CD70 scFv.

TABLE 14
Exemplary Sequences of PEBL element constructs comprising an anti-
CD70 scFv.

| Exemplary PEBL | | |
|---|---|---|
| Elements and | | SEQ ID |
| Domains | Amino Acid Sequence | NO: |
| PEBL-CD70-1 | MALPVTALLLPLALLLHAARPQVQLVQSGAEVKKPGASVKVSCKAS | 2645 |
| CD8α signal peptide, | GYTFTNYGMNWVRQAPGQGLKWMGWINTYTGEPTYADAFKGRVTMT | |
| CD70 scFv (1F6), | RDTSISTAYMELSRLRSDDTAVYYCARDYGDYGMDYWGQGTTVTVS | |
| ER-retention domain 1 | SGGGGSGGGGSGGGGSGDIVMTQSPDSLAVSLGERATINCRASKSV | |
| | STSGYSFMHWYQQKPGQPPKLLIYLASNLESGVPDRFSGSGSGTDF | |
| | TLTISSLQAEDVAVYYCQHSREVPWTFGQGTKVEIKGGGGSGGGGS | |
| | GGGGSGGGGSAEKDEL | |
| PEBL-CD70-2 | MALPVTALLLPLALLLHAARPQVQLVQSGAEVKKPGASVKVSCKAS | 2646 |
| CD8α signal peptide, | GYTFTNYGMNWVRQAPGQGLKWMGWINTYTGEPTYADAFKGRVTMT | |
| CD70 scFv (1F6), | RDTSISTAYMELSRLRSDDTAVYYCARDYGDYGMDYWGQGTTVTVS | |
| CD8α hinge, CD8α | SGGGGSGGGGSGGGGSGDIVMTQSPDSLAVSLGERATINCRASKSV | |
| TM, ER-retention | STSGYSFMHWYQQKPGQPPKLLIYLASNLESGVPDRFSGSGSGTDF | |
| domain 2 | TLTISSLQAEDVAVYYCQHSREVPWTFGQGTKVEIKKPTTTPAPRP | |
| | PTPAPTIASQPLSLRPEACRPAAGGAVHTRGLDFACDIYIWAPLAG | |
| | TCGVLLLSLVITLYKYKSRRSFIEEKKMP | |

B. ZFPs and ZFNs

In some embodiments, the CD70 inhibitor includes a DNA-binding protein such as one or more zinc finger protein (ZFP) or transcription activator-like protein (TAL), fused to an effector protein such as an endonuclease. Examples include ZFNs, TALEs, and TALENs.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al. Trends in Biotechnology: 31(7): 397-405, 2013). In some embodiments, commercially available zinc fingers are used or are custom designed. (See, e.g., Sigma-Aldrich catalog numbers CSTZFND, CSTZFN, CTil-1KT, and PZD0020).

C. TALs, TALEs and TALENs

In some embodiments, the CD70 inhibitor comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety.

In some embodiments, the CD70 inhibitor is a DNA binding endonuclease, such as a TALE nuclease (TALEN). In some aspects, the TALEN is a fusion protein comprising a DNA-binding domain derived from a TALE and a nuclease catalytic domain to cleave a nucleic acid target sequence.

In some embodiments, TALE repeats are assembled to specifically target a gene (e.g., CD70). A library of TALENs targeting 18,740 human protein-coding genes has been constructed (Kim et al. Nat. Struct. Mol. Biol. 20(12):1458-64, 2013). Custom-designed TALE arrays are commercially available through Cellectis Bioresearch (Paris, France), Transposagen Biopharmaceuticals (Lexington, Ky., USA), and Life Technologies (Grand Island, N.Y., USA).

In some embodiments the TALENs are introduced as trans genes encoded by one or more plasmid vectors. In some aspects, the plasmid vector can contain a selection marker which provides for identification and/or selection of cells which received said vector

D. Meganucleases and MegaTALs

In certain embodiments, the CD70 inhibitor comprises a meganuclease (homing endonuclease) or a portion thereof that exhibits cleavage activity. In some embodiments, a “meganuclease,” also referred to as a “homing endonuclease,” refers to an endodeoxyribonuclease characterized by a large recognition site (double stranded DNA sequences of about 12 to about 40 base pairs). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. Nos. 5,420,032; 6,833,252; Belfort et al. Nucleic Acids Res. 25:3379-3388, 1997; Dujon et al. Gene 82:115-118, 1989; Perler et al. Nucleic Acids Res. 22, 1125-1127, 1994; Jasin Trends Genet. 12:224-228, 1996; Gimble et al. J. Mol. Biol. 263:163-180, 1996; Argast et al. J. Mol. Biol. 280:345-353, 1998, and the New England Biolabs catalogue.

In any of the nucleases described herein, the nuclease can comprise an engineered TALE DNA-binding domain and a nuclease domain (e.g., endonuclease and/or meganuclease domain), also referred to as TALENs. Methods and compositions for engineering these TALEN proteins for robust, site-specific interaction with the target sequence of the user's choosing have been published (see U.S. Pat. No. 8,586,526). In some embodiments, the TALEN comprises an endonuclease (e.g., FokI) cleavage domain or cleavage half-domain. In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl. Acid Res.: 42(4):2591-601). In addition, the nuclease domain may also exhibit DNA-binding functionality.

E. RGENs (CRISPR/Cas Systems)

In some embodiments, the CD70 inhibitor is a DNA-binding nucleic acid, such as alteration via an RNA-guided endonuclease (RGEN). For example, the CD70 inhibitor can be a clustered regularly interspaced short palindromic repeats (CRISPR) and CRISPR-associated (Cas) protein. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides (e.g., a CD70 gene). The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence.” In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

The components of a CRISPR system can be implemented in any suitable manner, meaning that the components of such systems including the RNA-guided nuclease (e.g., Cas enzyme) and gRNA can be delivered, formulated or administered in any suitable form to the cells. For example, the RNA-guided nuclease may be delivered to a cell complexed with a gRNA (e.g., as a ribonucleoprotein (RNP) complex), the RNA-guided nuclease may be delivered to a cell separate (e.g., uncomplexed) to a gRNA, the RNA-guided nuclease may be delivered to a cell as a polynucleotide (e.g., DNA or RNA) encoding the nuclease that is separate from a gRNA, or both the RNA-guided nuclease and the gRNA molecule may be delivered as polynucelotides encoding each component.

One or more vectors driving expression of one or more elements of the CRISPR system can be introduced into the cell such that expression of the elements of the CRISPR system direct formation of the CRISPR complex at one or more target sites. Components can also be delivered to cells as ribonucleoprotein complexes, proteins, DNA, and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. In addition, a nucleic acid encoding the endonuclease (e.g., a Cas enzyme such as Cas8 or Cas9) may be delivered with gRNAs. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cash, Cas7, Cas8, Cas8a, Cas8b, Cas8c, Cas9 (also known as Csn1 and Csx12), Cas10, Cas10d, Cas12, Cas12a (Cpf1), Cas12b (C2c1), Cas12c (C2c3), Cas12d (CasY), Cas12e (CasX), Cas12f (Cas14, C2c10), Cas12g, Cas12h, Cas12i, Cas12k (C2c5), C2c4, C2c8, C2c9, Cas13, Cas13a (C2c2), Cas13b, Cas13c, Cas13d, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx11, Csx16, CsaX, Csx3, Csx1, Csx15, Csf1, Csf2, Csf3, Csf4, MAD7, GSU0054, homologs thereof, or modified versions thereof. These enzymes are known; for example, the amino acid sequence of S. pyogenes Cas9 protein may be found in the SWISSPROT database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some instances, the CRISPR enzyme can be Cas12a nuclease, such as MAD7. MAD7 is an engineered nuclease of the Class 2 type V-A CRISPR-Cas (Cas12a/Cpf1) family with a low level of homology to canonical Cas12a nucleases. MAD7 only requires a crRNA for gene editing and allows for specific targeting of AT rich regions of the genome. MAD7 cleaves DNA with a staggered cut as compared to S. pyogenes which has blunt cutting. The PAM sequence is YTTV, wherein Y indicates a C or T base, and V indicates A, C or G. The DNA cleavage sites for MAD7 relative to the target site are 19 bases after the YTTV PAM site on the sense strand and 23 bases after the complementary PAM site of the anti-sense strand.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Exemplary gRNA sequences for NR3CS (glucocorticoid receptor) include Ex3 NR3C1 sGl 5-TGC TGT TGA GGA GCT GGA-3 (SEQ ID NO: 687) and Ex3 NR3C1 sG2 5-AGC ACA CCA GGC AGA GTT-3 (SEQ ID NO: 688). Exemplary gRNA sequences for TGF-beta receptor 2 include EX3 TGFBR2 sGl 5-CGG CTG AGG AGC GGA AGA-3 (SEQ ID NO: 689) and EX3 TGFBR2 sG2 5-TGG-AGG-TGA-GCA-ATC-CCC-3 (SEQ ID NO: 690). The T7 promoter, target sequence, and overlap sequence may have the sequence TTAATACGACTCACTATAGG (SEQ ID NO: 691)+target sequence+gttttagagctagaaatagc (SEQ ID NO: 692).

In some embodiments the CD70 inhibitor comprises an RNA-guided endonuclease and a guide RNA (gRNA) targeting a CD70 gene. Exemplary gRNA sequences for CD70 comprise the nucleic acid sequence of SEQ ID NO: 2685, or SEQ ID NO: 2686. In some embodiments, the CD70 inhibitor comprises an RNA-guided endonuclease (e.g., a Cas enzyme such as Cas8 and Cas9) and a gRNA comprising the nucleic acid sequence of any one of SEQ ID Nos: 2682-2686 or 2883-2945.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA-binding domain (DBD) fusions, GAL4A DNA binding domain fusions, and herpes simplex virus (HSV) BP16 protein fusions. Additional domains that may form part of a fusion protein comprising a CRISPR enzyme are described in US Patent Appl. Publ. No. 2011/0059502, incorporated herein by reference.

In some embodiments, the immune cells (e.g., NK cells) of the present disclosure are modified by one or more methods described herein to have reduced levels of CD70. In particular, NK cells can be contacted with a CD70 inhibitor that is a nucleic acid (e.g., RNAi, siRNA, shRNA, tandem shRNA, and/or ribozymes) targeting CD70 mRNA, such that expression of CD70 is reduced or depleted in the genetically engineered NK cell as compared to the expression of CD70 in a control NK cell (e.g., a wild-type NK cell and/or a NK cell that has not been contacted with the CD70 inhibitor). In some instances, as compared to a control NK cell, CD70 expression or CD70 level in a NK cell that is contacted with a CD70 inhibitor is reduced by about 1% to about 100% (e.g., by about 1% to about 95%, about 1% to about 90%, about 1% to about 85%, about 1% to about 80%, about 1% to about 75%, about 1% to about 70%, about 1% to about 65%, about 1% to about 60%, about 1% to about 55%, about 1% to about 50%, about 1% to about 45%, about 1% to about 40%, about 1% to about 35%, about 1% to about 30%, about 1% to about 25%, about 1% to about 20%, about 1% to about 15%, about 1% to about 10%, about 1% to about 5%, about 10% to about 100%, about 10% to about 95%, about 10% to about 90%, about 10% to about 85%, about 10% to about 80%, about 10% to about 75%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 30% to about 100%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 40% to about 100%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 60% to about 100%, about 60% to about 95%, about 60% to about 90%, about 60% to about 85%, about 60% to about 80%, about 60% to about 75%, about 60% to about 70%, about 60% to about 65%, about 70% to about 100%, about 70% to about 95%, about 70% to about 90%, about 70% to about 85%, about 70% to about 80%, about 70% to about 75%, about 80% to about 100%, about 80% to about 95%, about 80% to about 90%, about 80% to about 85%, about 90% to about 100%, about 90% to about 95%, or about 95% to about 100%) as compared to the CD70 expression or CD70 level in a control NK cell (e.g., a wild-type NK cell and/or a NK cell that has not been contacted with the CD70 inhibitor). In certain instances, CD70 expression or CD70 level in a NK cell is determined about 3-10 days (e.g., 3, 4, 5, 6, 7, 8, 9 or 10 days) after the NK cell is contacted with the CD70 inhibitor. For example, in some instances, 3-10 days after a NK cell is contacted with a CD70 inhibitor, a CD70 level or expression in the NK cell is found to be reduced, as compared to a control NK cell (e.g., a wild-type NK cell and/or a NK cell that has not been contacted with the CD70 inhibitor).

### IV. Methods of Use

In some embodiments, the present disclosure provides methods for immunotherapy comprising administering an effective amount of the genetically engineered immune cells of the present disclosure. In some embodiments, a medical disease or disorder is treated by transfer of an immune cell population that elicits an immune response. In certain embodiments of the present disclosure, cancer or infection is treated by transfer of a genetically engineered immune cell population that elicits an immune response. Provided herein are methods for treating or delaying progression of cancer in an individual comprising administering to the individual an effective amount an antigen-specific population of genetically engineered immune cells (e.g., genetically engineered NK cells). The present methods may be applied for the treatment of immune disorders, solid cancers, hematologic cancers, and viral infections.

Tumors for which the present treatment methods are useful include any malignant cell type, such as those found in a solid tumor or a hematological tumor. In some embodiments, the cancer is a CD70-positive cancer. Exemplary solid tumors can include, but are not limited to, a tumor of an organ selected from the group consisting of pancreas, colon, cecum, stomach, brain (e.g., dysembryoplastic neuroepithelial tumor), head, neck, ovary (e.g., ovarian epithelial tumor), kidney, larynx, sarcoma, lung, bladder (e.g., bladder urothelial carcinoma), melanoma, prostate, and breast. Exemplary hematological tumors include tumors of the bone marrow, T or B cell malignancies (e.g., mature B cell neoplasms), leukemias (e.g., acute myeloid leukemia (AML)), lymphomas (e.g., non-Hodgkin's lymphoma), blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), pleural mesothelioma, cancer of the peritoneum, gastric or stomach cancer (including esophagogastric squamous cell carcinoma, stomach adenocarcinoma, gastrointestinal cancer and gastrointestinal stromal cancer), pancreatic cancer (e.g., pancreatic adenocarcinoma), cervical cancer (e.g., cervical squamous cell carcinoma and cervical adenocarcinoma), ovarian cancer, liver cancer (e.g., fibrolamellar carcinoma and hepatocellular carcinoma), bladder cancer, breast cancer (e.g., invasive breast carcinoma), colon cancer, colorectal cancer (e.g., colorectal adenocarcinoma), endometrial or uterine carcinoma, salivary gland carcinoma, kidney or renal cancer (e.g., renal clear cell carcinoma (RCC), renal non-clear cell carcinoma), prostate cancer (e.g., prostate adenocarcinoma), vulval cancer, thyroid cancer, various types of head and neck cancer (e.g., head and neck squamous cell carcinoma), esophageal cancer (e.g., esophageal squamous cell carcinoma), sarcoma, seminoma, non-seminomatous germ cell tumor, thymic epithelial tumor, glioblastoma, cholangiocarcinoma, adrenocortical carcinoma, glioma (e.g., encapsulated glioma and diffuse glioma), pheochromocytoma, and melanoma (e.g., ocular melanoma). In some embodiments, the present treatment methods are useful for treating an HTLV-1-associated malignancy or an EBV-associated malignancy.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's; paragranuloma; malignant lymphoma, small lymphocytic; malignant lymphoma, large cell, diffuse; malignant lymphoma, follicular; mycosis fungoides; other specified non-Hodgkin's lymphomas; B cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); myelodysplastic syndrome (MDS); chronic myeloblasts leukemia; diffuse large B cell lymphoma (DLBCL); peripheral T-cell lymphoma (PTCL); or anaplastic large cell lymphoma (ALCL).

Acute myeloid leukemia (AML) is a type of cancer in which the bone marrow makes abnormal myeloblasts. It is the most common form of acute leukemia in adults (Siegel et al. CA Cancer J. Clin. 64(1):9-29 (2014)). AML is a rapidly progressive disease with a median age at onset of 65 to 70 years. AML is known by many names, including acute myelocytic leukemia, acute myelogenous leukemia, acute granulocytic leukemia, and acute non-lymphocytic leukemia. “Acute” denotes the aggressive nature of this disease that can progress quickly, and if not treated, is fatal within a few months of diagnosis. The cancer originates in the bone marrow but rapidly spreads via the blood to other anatomical sites. The disease is observed in both children and adults, but is more common in the elderly. The chance of getting AML increases with age, but a person can get AML at any age. About 8 in 10 adults with acute leukemia have AML and about 1 in 6 children with leukemia will have AML. An average of 12,000 new cases of AML is expected on a yearly basis with approximately 30,000 patients living with or experiencing remission currently in the US.

Among elderly AML patients (>65 years of age), median survival is short (ranging from 3.9 months for patients 65 to 74 years of age to 1.4 months for patients>85 years of age). Treatment options for AML patients are limited, and outcomes are usually poor with an average 5-year survival rate of 20%, and less than a 5% 5-year survival rate for patients older than 65 (Thein et al. Cancer 119(15):2720-7, 2013). Certain subgroups of AML have a particularly worse outcome such as patients with abnormalities in chromosome 7, complex karyotype, relapsed and/or refractory AML and AML arising from antecedent myelodysplastic syndrome (MDS) or myeloproliferative neoplasms (MPNs). Patients aged 65 years and older with AML are more likely than younger patients to have unfavorable-risk cytogenetics. These cytogenetic factors are associated with resistance to chemotherapy and show considerably lower response rates to therapy. In addition to response rate, older patients with AML are often either not considered candidates for or choose not to receive standard induction chemotherapy because of poor tolerability and treatment outcomes. Induction chemotherapy is associated with high rates of treatment-related mortality and low complete response (CR) rates in this subset of patients. Currently, there are no approved therapies for AML patients who do not receive standard induction chemotherapy. The paucity of therapies and the poor response rates attributed to certain cytogenetic factors make AML an unmet medical need for new agents demonstrating clinical benefit with a favorable safety profile.

Hematopoiesis is characterized by the tissue specific hierarchical differentiation from pluripotent stem cells to more mature differentiated cellular phenotypes. Similar to the homeostatic hematopoiesis, AML is believed to arise form mutations accumulating in this quiescent stem cell population, which gives rise to the leukemic stem cell (LSC). The inability to eliminate this AML LSC population will result in relapse and therapeutic failure.

Although most patients with AML will achieve remission with induction chemotherapy; many will relapse, despite the administration of post-remission consolidation therapies. Relapses may occur weeks to many years later. Up to 10% of patients will be refractory to induction chemotherapy. Both of these groups of patients (relapsed/refractory) constitute a particularly poor risk group. Although an allogeneic stem cell transplant would be considered a recommended approach for those patients who respond to salvage therapies, it is feasible only in a small number of patients and is associated with significant morbidity and mortality (Hamadani et al. Biol. Blood Marrow Transplant. 5:556-67, 2008). In addition, outcomes for patients transplanted with refractory disease are poor (Duval et al. J. Clin. Oncol. 28(23):3730-8, 2010) and almost half of patients with relapsed disease are chemorefractory and thus not suitable for transplantation (Hamadani et al. Biol. Blood Marrow Transplant. 5:556-67, 2008), (Estey Am. J. Hematol. 88(4):318-27, 2013). Many novel drugs and approaches are being investigated for this group of patients. However, the CR rates have been, in general, less than 30% (Litzow et al. Br. J. Haemotol. 148:217-25, 2010), (Cortes et al. Cancer 118(2):418-27, 2012), (Kirschbaum et al. Leukemia 25(10): 1543-7, 2011).

In AML patients, cytogenetics are important prognostic factors in predicting response to treatment (Grimwade et al. Blood 92(7):2322-33, 1998). Patients with AML whose leukemic cells have translocations t(8;21), t(15; 17), t(16; 16), or inv(16) have a favorable outcome with induction chemotherapy and intensive post-remission consolidation chemotherapy. However, abnormalities of chromosomes 5 or 7,11q23 or complex karyotypes have a very poor outcome with currently available induction and post remission chemotherapy. Patients with a normal karyotype or with trisomy 8 have an intermediate prognosis. Among adults with AML, t(9;22) or t(4; 11) confer a very poor prognosis. Patients with t(9;22) AML are rarely, if ever, cured with chemotherapy alone. The immunophenotypic determination of surface antigens expressed on leukemic blast cells may aid in diagnosis and has important implications for treatment and prognosis of myeloid, T, and B lineage leukemias. Given that increases in long-term AML survival have proven elusive using conventional therapies, novel treatment strategies are needed.

Particular embodiments concern methods of treatment of leukemia. Leukemia is a cancer of the blood or bone marrow and is characterized by an abnormal proliferation (production by multiplication) of blood cells, usually immature white blood cells (leukocytes). It is part of the broad group of diseases called hematological neoplasms. Leukemia is a broad term covering a spectrum of diseases. Leukemia is clinically and pathologically split into its acute and chronic forms and/or by and the cell type of origin (myeloid or lymphoid).

In certain embodiments of the present disclosure, immune cells are delivered to an individual in need thereof, such as an individual that has cancer or an infection. The cells then enhance the individual's immune system to attack or directly attack the respective cancer or pathogenic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, 8, 9, 10, 11, or 12 or more weeks.

Certain embodiments of the present disclosure provide methods for treating or preventing an immune-mediated disorder. In some embodiments, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Bechcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erythematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or membranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosus, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine. An exemplary route of administering cyclophosphamide and fludarabine is intravenously. Likewise, any suitable dose of cyclophosphamide and fludarabine can be administered. In particular aspects, around 60 mg/kg of cyclophosphamide is administered for two days after which around 25 mg/m² fludarabine is administered for five days.

In some embodiments, the subject can be administered nonmyeloablative lymphodepleting immunotherapy prior to the genetically engineered immune cells (e.g., genetically engineered NK cells). The nonmyeloablative lymphodepleting immunotherapy can be any suitable such therapy, which can be administered by any suitable route. The nonmyeloablative lymphodepleting immunotherapy can comprise, for example, the administration of an anti-CD52 agent or anti-CD20 agent. In some embodiments, the lymphodepleting immunotherapy is an anti-CD52 antibody. In some embodiments, the anti-CD52 antibody is alemtuzumab. In some embodiments, the lymphodepleting immunotherapy is an anti-CD20 antibody. Exemplary anti-CD20 antibodies include, but are not limited to rituximab, ofatumumab, ocrelizumab, obinutuzumab, ibritumomab or iodine i131 tositumomab. An exemplary route of administering anti-CD52 agent or anti-CD20 agent is intravenously. Likewise, any suitable dose of anti-CD52 agent or anti-agent can be administered.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective amounts of genetically engineered immune cells can be administered by a number of routes, including parenteral administration, for example, intravenous, intraperitoneal, intramuscular, intrasternal, or intraarticular injection, or infusion.

The therapeutically effective amount of genetically engineered immune cells for use in adoptive cell therapy is that amount that achieves a desired effect in a subject being treated. For instance, this can be the amount of genetically engineered immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The genetically engineered immune cell population can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of genetically engineered immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ genetically engineered immune cells/m². In a certain embodiment, the dose used in the treatment of human subjects ranges from about 3.8×10⁹ to about 3.8×10¹⁰ genetically engineered immune cells/m². In additional embodiments, a therapeutically effective amount of genetically engineered immune cells can vary from about 5×10⁶ cells per kg body weight to about 7.5×10⁸ cells per kg body weight, such as from about 2×10⁷ cells to about 5×10⁸ cells per kg body weight, or from about 5×10⁷ cells to about 2×10⁸ cells per kg body weight, or from about 5×10⁶ cells per kg body weight to about 1×10⁷ cells per kg body weight. In some embodiments, a therapeutically effective amount of genetically engineered immune cells ranges from about 1×10⁵ cells per kg body weight to about 10×10⁹ cells per kg body weight. The exact amount of genetically engineered immune cells is readily determined by one of skill in the art based on the age, weight, sex, and physiological condition of the subject. Effective doses can be extrapolated from dose-response curves derived from in vitro or animal model test systems.

The genetically engineered immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetyls alicylic acid, ibuprofen or naproxen sodium), cytokine antagonists (for example, anti-TNF and anti-IL-6), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the genetically engineered immune cells, depending on the desired effect. This administration of the genetically engineered immune cells and the agent can be by the same route or by different routes, and either at the same site or at a different site.

### 1. Pharmaceutical Compositions

Also provided herein are pharmaceutical compositions and formulations comprising genetically engineered immune cells (e.g., NK cells) and a pharmaceutically acceptable carrier.

In some embodiments, a pharmaceutical composition comprises a dose ranging from about 1×10⁵ NK cells to about 1×10⁹ NK cells. In some embodiments, the dose is about 1×10⁵, 1×10⁶, 1×10⁷, 1×10⁸ or 1×10⁹NK cells. In some embodiments, a pharmaceutical composition comprises a dose ranging from about 5×10⁵ NK cells to about 10×10¹² NK cells.

In some embodiments, a pharmaceutical composition is cryopreserved. In some embodiments, at least 10, at least 15, at least 20, at least 25, at least 30, at least 35, at least 40, at least 45, at least 50, at least 55, at least 60, at least 65, at least 70, at least 75, at least 80, at least 85, at least 90, at least 91, at least 92, at least 93, at least 94, at least 95, at least 96, at least 97, at least 98, at least 99% of the genetically engineered NK cells in the cryopreserved pharmaceutical composition specifically bind human CD70 after thawing.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as polyethylene glycol (PEG).

### 2. Combination Therapies

In some embodiments, the compositions and methods of the present embodiments involve a genetically engineered immune cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

A genetically engineered immune cell may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below a genetically engineered immune cell is “A” and an anti-cancer therapy is “B”: A/B/A B/A/B B/B/A A/A/B A/B/B B/A/A A/B/B/B B/A/B/B B/B/B/A B/B/A/B A/A/B/B A/B/A/B A/B/B/A B/B/A/A B/A/B/A B/A/A/B A/A/A/B B/A/A/A A/B/A/A A/A/B/A

Administration of any compound, therapy, or genetically engineered immune cell of the present embodiments to a patient will follow general protocols for the administration of such compounds, therapies, and immune cells taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclophosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, decitabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine,plicomycin, gemcitabien, navelbine, farnesyl-protein tansferase inhibitors, transplatinum, and pharmaceutically acceptable salts, acids, or derivatives of any of the above. In some embodiments, azacitidine is administered at 75 mgs/m² subcutaneously.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

In some embodiments, the additional immunotherapy for use in combination or in conjunction with the methods described herein is an antibody-drug conjugate (e.g., brentuximab vedotin (ADCETRIS) and trastuzumab emtansine or T-DM1 (KADCYLA).

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

In some embodiments, the additional immunotherapy for use in combination or in conjunction with the methods described herein is an immune adjuvant, e.g., Mycobacterium bovis, Plasmodium falciparum, dinitrochlorobenzene, and aromatic compounds (U.S. Pat. Nos. 5,801,005 and 5,739,169; Hui and Hashimoto, Infect. Immun. 66(11):5329-36, 1998; Christodoulides et al. Microbiology (Reading) 144 (Pt 11):3027-37, 1998); a cytokine therapy, e.g., interferons α, β, and γ, IL-1, GM-CSF, and TNF (Bukowski et al. Clin. Cancer Res. 4(10): 2337-47, 1998; Davidson et al. J Immunother. 21(5): 389-9, 1998; Hellstrand et al. Acta Oncol. 37(4): 347-53, 1998); a gene therapy, e.g., TNF, IL-1, IL-2, and p53 (Qin et al. Proc. Nat'l. Acad. Sci. USA 95(24):14411-6, 1998; Austin-Ward and Villaseca, Rev. Med. Chil. 126(7): 838-45, 1998; U.S. Pat. Nos. 5,830,880 and 5,846,945); and a monoclonal antibody(ies), e.g., anti-CD20, anti-ganglioside GM2, and anti-p185 (Hollander Front Immunol. 3:3, 2012; Hanibuchi et al. Int. J. Cancer 78(4):480-5, 1998; U.S. Pat. No. 5,824,311). It is contemplated that one or more anti-cancer therapies may be employed with the antibody therapies described herein.

In some embodiments, the immunotherapy for use in combination or in conjunction with the methods described herein may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO 2015/016718; Pardoll Nat. Rev. Cancer 12(4):252-64, 2012; both incorporated herein by reference). Known inhibitors of the immune checkpoint proteins or analogs thereof may be used, in particular chimerized, humanized or human forms of antibodies may be used (e.g., pembrolizumab). As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. 2014/0294898, 2014/022021, and 2011/0008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDLL or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO 2006/121168. Pembrolizumab, also known as MK-3475, Merck 3475, lambrolizumab, KEYTRUDA®, and SCH-900475, is an anti-PD-1 antibody described in WO 2009/114335. CT-011, also known as hBAT or hBAT-1, is an anti-PD-1 antibody described in WO 2009/101611. AMP-224, also known as B7-DCIg, is a PDL2-Fc fusion soluble receptor described in WO 2010/027827 and WO 2011/066342.

Another immune checkpoint that can be targeted in the methods provided herein is the cytotoxic T-lymphocyte-associated protein 4 (CTLA-4), also known as CD 152. The complete cDNA sequence of human CTLA-4 has the GENBANK accession number L15006. CTLA-4 is found on the surface of T cells and acts as an “off switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., Proc. Natl. Acad. Sci. U.S.A. 95(17):10067-10071, 1998; Camacho et al., Clin. Oncology 22(145): Abstract No. 2505 (antibody CP-675206), 2004; and Mokyr et al., Cancer Res. 58:5301-5304, 1998 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO 2001/014424, WO 2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in some embodiments, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO 1995001994 and WO 1998/042752; all incorporated herein by reference, and immunoadhesins such as described in U.S. Pat. No. 8,329,867, incorporated herein by reference.

Examples of immunotherapies for use in treatment of kidney cancer or renal cell cancer include, but are not limited to Afinitor (Everolimus), Afinitor Disperz (Everolimus), Aldesleukin, Avastin (Bevacizumab), Avelumab, Axitinib, Bavencio (Avelumab), Bevacizumab, Cabometyx (Cabozantinib-S-Malate), Cabozantinib-S-Malate, Everolimus, IL-2 (Aldesleukin), Inlyta (Axitinib), Interleukin-2 (Aldesleukin), Ipilimumab, Keytruda (Pembrolizumab), Lenvatinib Mesylate, Lenvima (Lenvatinib Mesylate), Mvasi (Bevacizumab), Nexavar (Sorafenib Tosylate), Nivolumab, Opdivo (Nivolumab), Pazopanib, Hydrochloride, Pembrolizumab, Proleukin (Aldesleukin), Sorafenib Tosylate, Sunitinib Malate, Sutent (Sunitinib Malate), Temsirolimus, Torisel (Temsirolimus), Votrient (Pazopanib Hydrochloride), Yervoy (Ipilimumab).

Examples of immunotherapies for use in treatment of Acute Myeloid Leukemia (AML) include, but are not limited to Azacitidine, Arsenic Trioxide, Cerubidine (Daunorubicin Hydrochloride), Cyclophosphamide, Cytarabine, Daunorubicin Hydrochloride, Daunorubicin Hydrochloride and Cytarabine Liposome, Daurismo (Glasdegib Maleate), Dexamethasone, Doxorubicin Hydrochloride, Enasidenib Mesylate, Gemtuzumab Ozogamicin, Gilteritinib Fumarate, Glasdegib Maleate, Idamycin PFS (Idarubicin Hydrochloride), Idarubicin Hydrochloride, Idhifa (Enasidenib Mesylate), Ivosidenib, Midostaurin, Mitoxantrone Hydrochloride, Mylotarg (Gemtuzumab Ozogamicin), Rubidomycin (Daunorubicin Hydrochloride), Rydapt (Midostaurin), Tabloid (Thioguanine), Thioguanine, Tibsovo (Ivosidenib), Trisenox (Arsenic Trioxide), Venclexta (Venetoclax), Venetoclax, Vincristine Sulfate, Vyxeos (Daunorubicin Hydrochloride and Cytarabine Liposome), Xospata (Gilteritinib Fumarate).

### 3. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 4. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. Dosage Regimens

In some embodiment, the genetically engineered immune cells (e.g., genetically engineered NK cells) are modified by engineering (e.g., genetically modified) to introduce a chimeric antigen receptor (e.g., anti-CD70 CAR) and a cytokine (e.g., IL-15 or a mbIL-15/IL-15RA complex) (or nucleic acids encoding these proteins) into the cells and then rapidly infused into a subject. In some embodiments, immune effector cells are modified by engineering/introducing a chimeric receptor, and functional effector element and/or and a cytokine into the cells and then infused within about 0 days, within about 1 day, within about 2 days, within about 3 days, within about 4 days, within about 5 days, within about 6 days or within about 7 days into a subject.

In some embodiments, an amount of genetically engineered immune cellsis administered to a subject in need thereof and the amount is determined based on the efficacy and the potential of inducing a cytokine-associated toxicity. In another embodiment, the cells are CAR′ and CD56⁺ cells. In some embodiments, an amount of the cells comprises about 10⁴ to about 10⁹ cells/kg. In some cases, an amount of cells comprises about 10⁴ to about 10⁵ cells/kg. In some cases, an amount of cells comprises about 10⁵ to about 10⁶ cells/kg. In some cases, an amount of genetically engineered immune cells comprises about 10⁶ to about 10′ cells/kg. In some cases, an amount of genetically engineered immune cells comprises about 10′ to about 10⁸ cells/kg. In some cases, an amount of genetically engineered immune cells comprises about 10⁸ to about 10⁹ cells/kg. In some cases, am amount of genetically engineered immune cells comprises about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, or about 1×10⁹ cells/kg.

In some embodiments, the genetically engineered immune cells are targeted to the cancer via regional delivery directly to the tumor tissue. For example, in ovarian or renal cancer, the genetically engineered immune cells can be delivered intraperitoneally (IP) to the abdomen or peritoneal cavity. Such IP delivery can be performed via a port or pre-existing port placed for delivery of chemotherapy drugs. Other methods of regional delivery of genetically engineered immune cells can include catheter infusion into resection cavity, ultrasound guided intratumoral injection, hepatic artery infusion or intrapleural delivery.

In some embodiments, a subject in need thereof, can begin therapy with a first dose of genetically engineered immune cells delivered via IV followed by a second dose of genetically engineered immune cells delivered via IV. In some embodiments, a subject in need thereof, can begin therapy with a first dose of genetically engineered immune cells delivered via IP followed by a second dose of genetically engineered immune cells delivered via IV. In a further embodiment, the second dose of genetically engineered immune cells can be followed by subsequent doses which can be delivered via IV or IP. In some embodiments, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In some embodiments, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the duration between the first and second or further subsequent dose can be about: 0, 1, 2, 3, 4, 5, 6, 7, 8, 9 or 10 years.

In another embodiment, a catheter can be placed at the tumor or metastasis site for further administration of 1, 2, 3, 4, 5, 6, 7, 8, 9, 10 doses of genetically engineered immune cells. In some cases, doses of genetically engineered immune cells can comprise about 10² to about 10⁹ cells/kg. In cases where toxicity is observed, doses of genetically engineered immune cells can comprise about 10² to about 10⁵ cells/kg. In some cases, doses of genetically engineered immune cells can start at about 10² cells/kg and subsequent doses can be increased to about: 10⁴, 10⁵, 10⁶, 10⁷, 10⁸ or 10⁹ cells/kg.

### VI. Articles of Manufacture or Kits

An article of manufacture or a kit is provided comprising genetically engineered immune cells is also provided herein. The article of manufacture or kit can further comprise a package insert comprising instructions for using the genetically engineered immune cells to treat or delay progression of cancer in an individual or to enhance immune function of an individual having cancer. Any of the genetically engineered immune cells described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### EXAMPLES

### Example 1—Methods of Modifying NK Cells to Express Cars Comprising CD70 Binding Sequences

NK Cell Derivation from CAR-Expressing iPSCs

The derivation of NK cells from iPSCs and CAR transfected iPSCs have been previously described (Knorr et al., 2013 (supra); Ng et al. Nat Protoc. 3:768-76, 2008). Briefly, 3,000 TrypLE-adapted iPSCs are seeded in 96-well round-bottom plates with APEL culture (Ng et al., 2008, supra) containing 40 ng/ml human Stem Cell Factor (SCF), 20 ng/ml human Vascular Endothelial Growth Factor (VEGF), and 20 ng/ml recombinant human Bone Morphogenetic Protein 4 (BMP-4). After day 11 of hematopoietic differentiation, spin embryoid bodies (EBs) are then directly transferred into each well of uncoated 24-well plates under a condition of NK cell culture. Cells are then further differentiated into NK cells as previously reported (Bachanova et al. Blood 123(25): 3855-63 2014; Ni et al. Methods Mol. Biol. 1029: 33-41 2013) using 5 ng/mL IL-3 (first week only), 10 ng/mL IL-15, 20 ng/mL IL-7, 20 ng/mL SCF, and 10 ng/mL flt3 ligand for 28-32 days. Half-media changes are performed weekly. NK cells are harvested for irradiated mbIL-21 expressing artificial antigen presenting cells (aAPCs) expansion (Denman et al. PLoS One 7(1):e30264, 2012) with 50 units/mL of hIL-2.

### Molecular Constructs

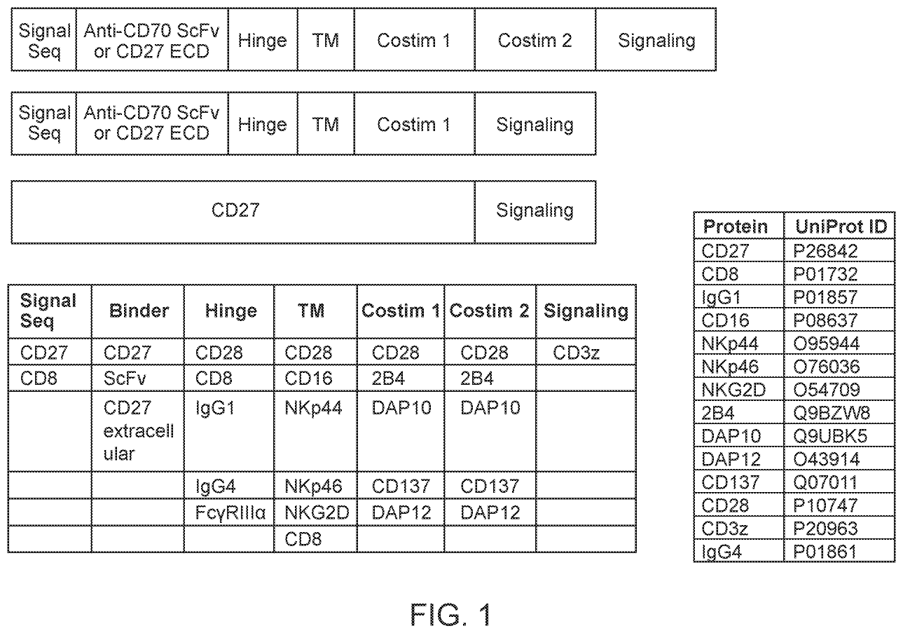

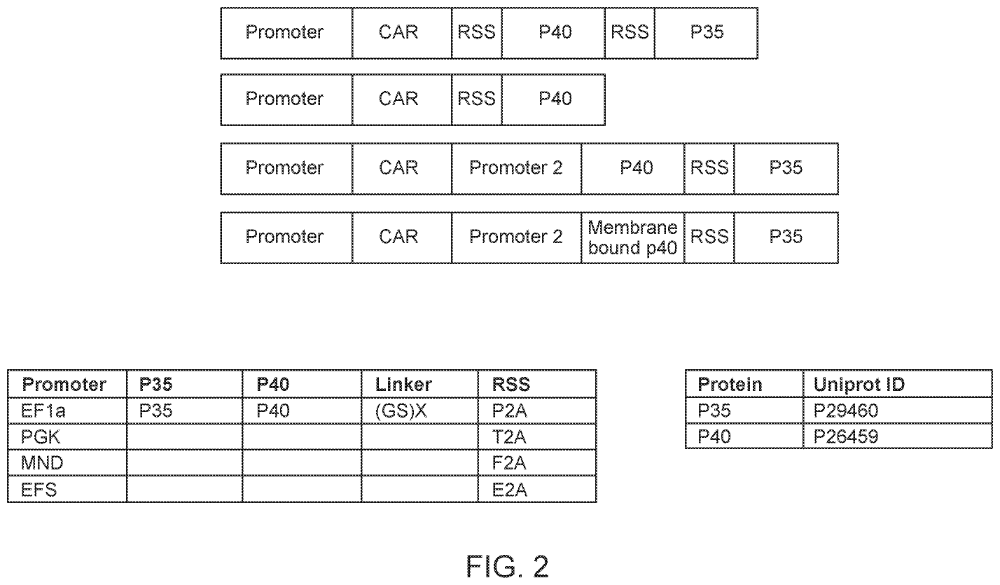

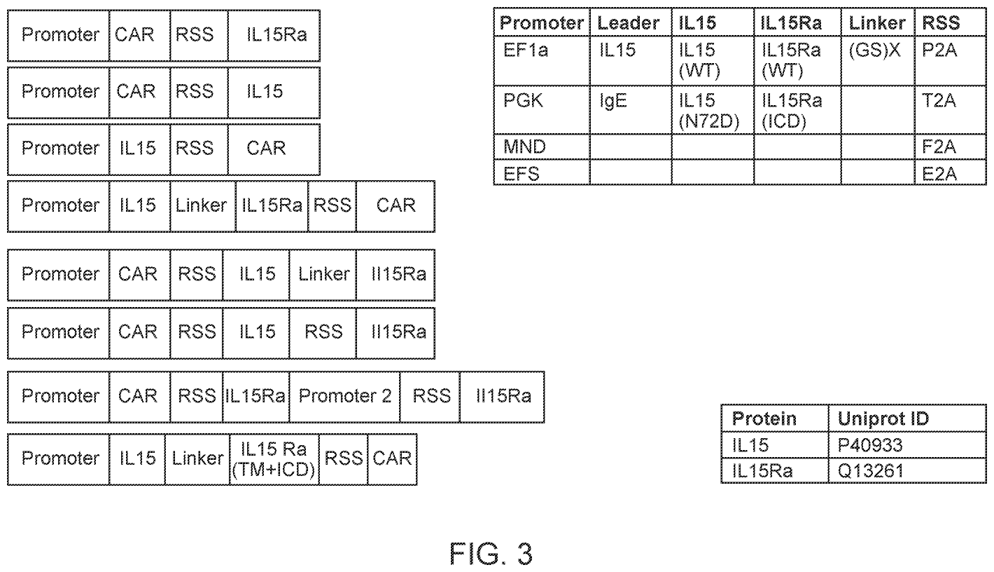

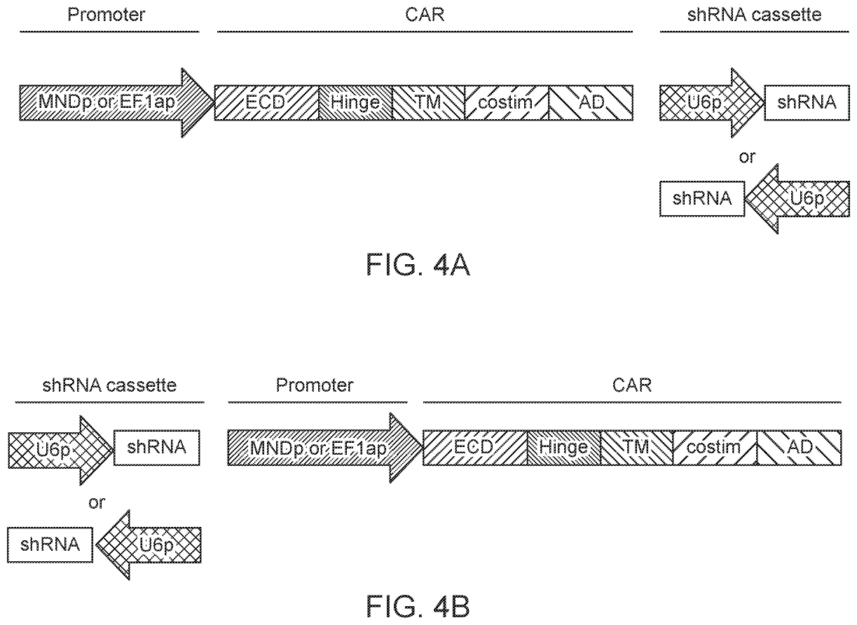

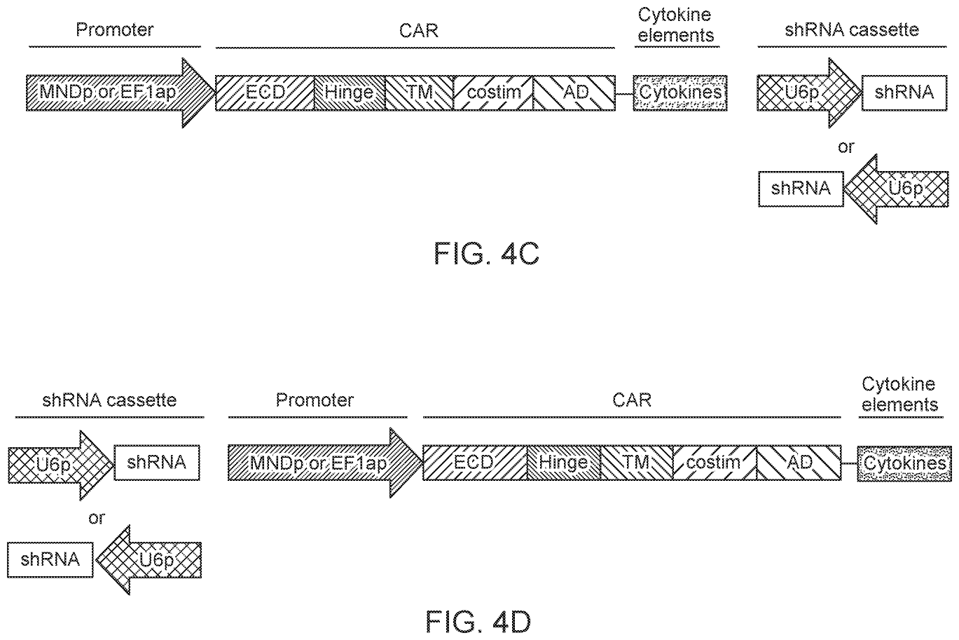

TcBuster Transposon vectors are designed and reconstructed as previously described. Transgene expression is driven by the mCAG promoter. CARs constructs are designed to bind specifically to CD70. FIG. 1 shows a schematic diagram of the structure of each anti-CD70 CAR tested. Membrane bound IL-15, co-expressed IL-15/IL-15RA polypeptides and membrane bound IL-12 polypeptides are also constructed (FIGS. 2 and 3). CARs and mbIL-15 and mbIL-12 are synthesized as gBlocks gene fragment and cloned into the transposon using restriction enzyme cloning and ligation. Correct CAR sequences are confirmed by restriction enzyme digest and sequencing analyses. Insulated TcBuster vectors are generated by PCR of CAR expression cassettes from TcBuster transposon vectors and subsequent BP Clonase reaction into pDONR221 to generate pENTR221-CAR cassette plasmids. pENTR221-CAR cassette plasmids are subsequently used for LR Clonase reaction into PB-I-DEST-I to generate final TcBuster expression vectors. The PB-I-DEST-I vector contains a 2.4 kb cHS4 insulator (I) flanking the Gateway destination cassette (DEST) used for LR clonase cloning. Generation of stable clone of CAR transfected NK cells are performed as described above. To determine the copy numbers of integrated vector, genomic DNA is isolated from the iPSCs and NK92 cells and performed quantitative PCR using sets of primers specific for GFP:zeo region of vector and for the human RNase gene. To determine absolute value, a standard curve is generated using serial dilutions of a plasmid containing GFP:zeo region. Reactions are carried out in triplicate in CFX384 Touch™ Real-Time PCR Detection System.

Nucleic acids/DNA/genes encoding the constructs are cloned into the multiple cloning site of retroviral gene transfer vectors: pELNS or pES.12-6(g)ps under control of one of the following promoters: EF-1, EF1a, EFS, MND, MSCV, CMV, PGK or RPBSA. Retrovirus is produced in 293T cells by transfecting the cells with gene transfer vectors. Cells are placed in fresh culturing medium. The virus supernatant is collected 48-72 hours post-medium change by centrifugation at 800×g for 5 minutes. The supernatant is collected, filtered, and frozen in aliquots at −80° C.

### Quantitative RT-PCR

To test the level of chimeric antigen receptor transcript expression in genetically engineered NK cells, RNA are processed from NK cells at harvest. For cell cycle gene analysis, transcripts are evaluated using the Human Cell Cycle RT² Profiler PCR Array (Qiagen). CPT1a, SOCS1, SOCS2, and SOCS3 transcripts are analyzed and normalized to GAPDH.

### Immunoblot

To test the chimeric antigen receptor expression in genetically engineered NK cells, suspension cells are lysed in RIPA lysis buffer with fresh protease inhibitor cocktail on ice for 20 min and sonicated for 2 seconds on ice. Membrane proteins are extracted using a Membrane Protein Extraction Kit. Sample proteins are measured by a standard bicinchoninic acid assay, size fractioned by polyacrylamide gel electrophoresis (PAGE), and are transferred to nitrocellulose membrane. Nonspecific binding are blocked by incubating in TBST 5% BSA plus 1% Triton X-100 solution for 1 hours, followed by incubation with primary antibodies, overnight at 4° C. Species specific IRDye-conjugated secondary antibodies (1:10,000) are applied to membranes for 1 hour at room temperature. Immunoreactive products are visualized in Odyssey Imaging Systems. All loading samples are normalized by staining of GAPDH.

### Proliferation Assays

To test the proliferation and viability of NK cells, genetically engineered NK cells or NK cells from healthy donors are labeled with Cell Proliferation Dye and placed in the continuous IL-15 treatment (IL-15cont) or intermittent IL-15 treatment (IL-15gap) conditions for 9 days (e.g., as described in Felices et al. (2018) JCI Insight 3(3): e96219). In the IL-15cont treatment, cells are cultured in media supplemented IL-15 during 3 consecutive 3-day cycles. In the IL-15gap treatment, cells are cultured in media for an initial 3-day cycle in media supplemented with IL-15, in media without IL-15 for a 3-day cycle, and subsequently in media supplemented with IL-15 for a 3-day cycle. Viable NK cells (CD56⁺CD3⁻) are then analyzed for dilution of dye.

### Cell Lysis Assay

To test the ability of the genetically engineered NK cells to specifically target cells for lysis, human AML cell lines are incubated with ⁵¹chromium (⁵¹Cr) or europium for 1 hour at 37° C., washed three times, and cocultured with NK cells at the indicated effector to target (E:T) ratios. Total lysis (test release) is achieved with the use of 5% Triton-X 100. After a period of incubation, cells are harvested and analyzed. Specific ⁵¹/Cr or europium/lysis is determined following the equation: Percentage of specific lysis=100×(Test release−Spontaneous release)/(Maximal release−Spontaneous release).

### CD107a (LAMP1) Expression and IFN-γ Staining

CD107a (LAMP1) expression and IFN-γ production by target cells are two proxys for the level of NK cell binding with said cells. NK cells are incubated with or without cancer target cell lines (K562 cells, K562meso cells, MA148, cells, or A1847 cells) at 1:2 effector to target ratios. CD107a-APC antibody is added to each well and allowed to incubate for 1 hour, and subsequently GolgiStop and GolgiPlug is added for an additional 2-hour incubation. At the completion of incubation, cells are washed with FACS buffer and are stained with CD56-PE and LIVE/DEAD Fixable Aqua Sstain (ThermoFisher Scientific). Cells are then fixed with fixation buffer for 10 minutes on ice, following by permeabilization with permeabilization/wash buffer for 10 minutes at 4° C. Cells are washed and stained with interferon-γ (IFN-γ)-Pacific Blue for 30 minutes at 4° C., and then final washed for analysis. CD107a expression and intracellular IFN-γ production are evaluated by normalization data of NK cell without target cell co-culture.

### Metabolic Studies

CAR+NK cells are harvested at day 9 of culture and resuspended in Seahorse XF Assay Medium (Agilent Technologies). One million cells/well are immobilized with Poly-L-Lysine (MilliporeSigma). The extracellular acidification rate and the oxygen consumption rate are measured (pmoles/min) in real time in an XFe24 analyzer after injection of glucose (10 mM), oligomycin (1 μM), FCCP (1 μM) plus sodium pyruvate (1 mM), and rotenone/antimycin A (0.5 μM). SRC is calculated from the change from basal oxygen consumption, after addition of glucose, to maximal oxygen consumption, after addition of FCCP. In experiments measuring the input of FAO, glucose is added to the media prior to beginning measurements. This is the followed by injection of the CPT-1 inhibitor etomoxir, injection of oligomycin, injection of FCCP plus sodium pyruvate, and final injection of rotenone/antimycin A.

AML Xenografts with NK Cell Treatment

AML cancer cells are incorporated into a previously described NK cell xenogeneic mouse model system (Hermanson et al. Stem Cells 34(1):93-101, 2016). NOD-scid IL2rγ null (NSG, n=5/group) mice are obtained from Jackson Laboratories for all in vivo experiments. Human leukemia cell lines include Kasumi-1 (Asou et al. Blood 77(9):2031-6 1991), HL-60 (Gallagher et al. Blood 54(3): 713-33, 1979), PL-21 (Kubonishi et al. Blood 63(2):254-9. 1984), NB4 (Lanotte et al. Blood 77(5): 1080-6 1991), HT-93 (Kishi et al. Exp. Hematol. 26(2):135-42, 1998), U-937 (Sundstrom and Nilsson, Int. J Cancer 17(5):565-771976), MV4-11 (Lange et al. Blood 70: 192-9, 1987), MOLM-13 (Matsuo et al. Leukemia 11(9):1469-77 1997), NOMO-1 (Kato et al. Acta Haematol Jpn. 49:277, 1986), KG-1 (Koeffler and Golde Science 200:1153-4, 1978), and HEL (Martin and Papayannopoulou, Science 216(4551):1233-5, 1982). Mice are given 2×10⁵ of luciferase expressing cancer cells intraperitoneally (I.P.) 4 days prior to NK cell infusion (Day −4). On Day −1 mice are conditioned with 225 cGy, and bioluminescent imaging (BLI) is used to normalize tumor engraftment burden in each group. 1.5×10⁷ or 1.0×10⁷ cells per mouse NK cells or T cells are then given intraperitoneally on Day 0. Cytokine administration of hIL-2 (10,000 unit/mouse, every 2-3 day for 21 days) and hIL-15 (10 ng/mouse for 7 days) is initiated on mice under NK cell therapy after day 1. Tumor aggressiveness is determined by BLI weekly using the Xenogen IVIS Imaging system.

### In Vivo Mouse Study and Imaging

NOD/SCID/γc−/− (NSG) mice (Jackson Labs) are sublethally irradiated (275 cGy) and xenografted i.v. with 750,000 firefly luciferase-expressing human leukemia cell lines (e.g., HL-60 human acute promyelocytic leukemia cells) (day −3). At day 0, mice are given i.v. 1×10⁶ IL-15cont or IL-15gap NK cells; they are harvested at day 9 of culture. 2 μg IL-15 (NCI) is injected i.p. per mouse on that day and every 7 days following to induce basal maintenance of the NK cells. Retro orbital bleeds, 150 μl, are carried out at day 6, 13, and 20 to assess human cell content. Mice are injected with 100 μl of 30 mg/mL luciferin substrate 10 minutes prior to imaging and then anesthetized via inhalation of isoflurane gas. Assessment of the presence of tumor cells by bioluminescent imaging (BLI) is carried out at day 14 using the Xenogen IVIS imaging system and analyzed with Living Image 2.5 software (Caliper Life Science).

### Example 2—Genetically-Engineered Car NK Cells Derived from Peripheral Blood or Cord Blood NK Cells

Isolation of NK Cells from Peripheral Blood or Cord Blood

NK cells are isolated from either human peripheral blood leukapheresis samples or cord blood units. Briefly, leukapheresis samples or cord blood units are enriched for peripheral blood mononuclear cells (PBMCs). One method for PBMC enrichment is separation using a Ficoll density gradient. Next, peripheral blood NK cells are isolated from PBMC samples using immunomagnetic separation beads. Beads are conjugated to a cocktail of specific immunophenotypic antibodies to enable NK cell isolation through either positive or negative selection. Isolated NK cells are activated prior to transduction. One method for NK cell activation is co-culture with irradiated artificial antigen presenting cells (aAPCs) expressing mbIL-21 and 4-1BBL for expansion in the presence of hIL-2.

### Molecular Constructs

For viral-based cell engineering CD70 CAR constructs described in FIG. 1 are cloned into the multiple cloning site of retroviral gene transfer vectors: pELNS or pES.12-6(g)ps under control of one of the following promoters: EF-1, EF1a, EFS, MND, MSCV, CMV, PGK, mCAG or RPBSA. Retrovirus is produced in 293T cells by transfecting the cells with gene transfer vectors. Cells are placed in fresh culturing medium. The virus supernatant is collected 48-72 hours post-medium change by centrifugation at 800×g for 5 minutes. The supernatant is collected, filtered, and frozen in aliquots at −80° C. Non-viral gene delivery system is based on TcBuster Transposon. Transgene expression is driven by one of the following promoters: EF-1, EF1a, EFS, MND, MSCV, CMV, PGK, mCAG or RPBSA. CD70 CAR constructs are cloned into transposon vectors using SpeI/NheI restriction sites. Transposon DNA and mRNA encoding TcBuster transposase are co-delivered into NK cells via electroporation with MaxCyte or Neon machine. Successful integration and expression efficiency are assessed post transduction by flow cytometry to characterize CAR expression.

### Cell Lysis Assay

Genetically modified peripheral blood NK cells are then assessed for functionality in cell killing assays. One method to test the ability of the modified NK cells to specifically target cells for lysis is co-culture with human AML or other CD70-positive tumor cell lines expressing luciferase. Cell killing is characterized across a range of effector to target ratios (E:T). As a negative control, luciferase expressing cell lines are cultured with unmodified NK cells or NK cells expressing a non-targeting construct. An additional control is culture of luciferase expressing cell lines in the absence of NK cells. After a period of co-culture, luciferase signal is analyzed and compared to control samples. Target cell killing is observed as the decrease in luciferase signal in target cells relative to controls. Alternatively, target cell killing is observed as the release of luciferase into cell culture media.

### CD107a and Cytokine Expression

CD107a expression and cytokines such as IFNγ and TNFα by NK are assessed to characterize functionality. Genetically modified NK cells are co-cultured with human AML, or other CD70-positive tumor cell lines across a range of E:T ratios for a period of time. Cell surface expression of CD107a is assessed by flow cytometry with a CD107a-specific antibody. Cytokine expression is assessed by intracellular cytokine staining. Briefly, samples are treated with a protein transport inhibitor such as GolgiStop for a period of time. Next, samples are treated with a fixation/permeabolization solution, stained with cytokine-specific antibodies, and assessed by flow cytometry. Alternatively, cytokine secretion into cell culture can be measured through multiplex ELISA. CD107a and cytokine expression are evaluated relative to controls including unmodified NK cells, NK cells expressing a non-targeting construct, and modified NK cells in the absence of target cells.

### Proliferation Assays

Proliferation of modified NK cells is assessed following co-culture with human AML or other CD70-positive tumor cell lines for a period of time. One method is covalent labeling of viable NK cells with a cell proliferation dye such as CFSE, where proliferation corresponds to dilution of dye. Alternatively, proliferation of modified NK cells is assessed by flow cytometry to determine NK cell counts. NK cells are labeled with NK-specific phenotypic markers and are negative for other lineage phenotypic markers. For both methods, proliferation of modified NK cells is compared to controls including unmodified NK cells, NK cells expressing a non-targeting construct, and modified NK cells in the absence of target cells.

### Example 4—CD70 Knockout NK Cells Exhibit Increased Expansion and Viability and NK Knockout Enables the Generation of Anti-CD70 CARs in NK Cells

### Activation of NK Cells Induces a Significant Increase in CD70 Expression

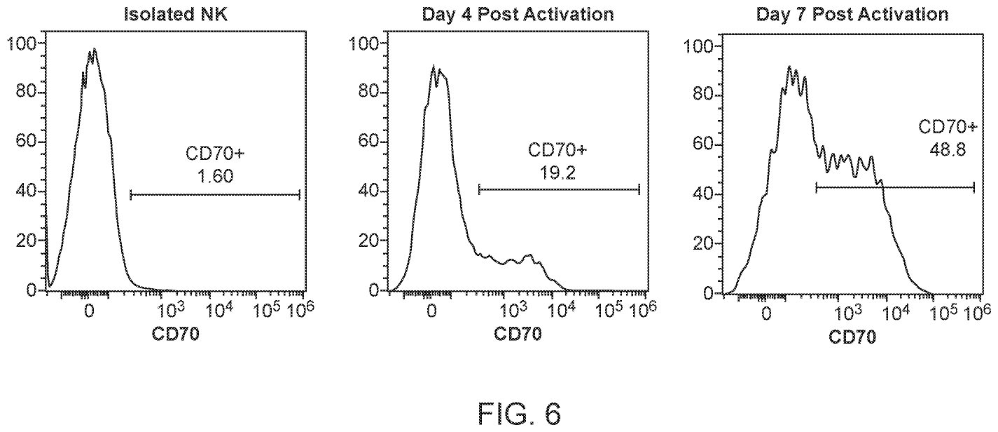

Peripheral blood NK cells were isolated from human leukapheresis samples utilizing magnetic isolation. NK cells were then negatively selected from PBMC samples using immunomagnetic separation beads. Isolated NK cells were cultured with irradiated artificial antigen presenting cells (aAPCs) prior to transduction to enable NK cell activation and expansion in the presence of 100 IU/mL of recombinant IL-2. Following activation, the level of CD70 expression was assessed by flow cytometry 4 days and 7 days post-activation. As shown in FIG. 6, the expression of CD70 protein dramatically increases following activation.

### CD70 Knockout Improves NK Cell Expansion and Viability

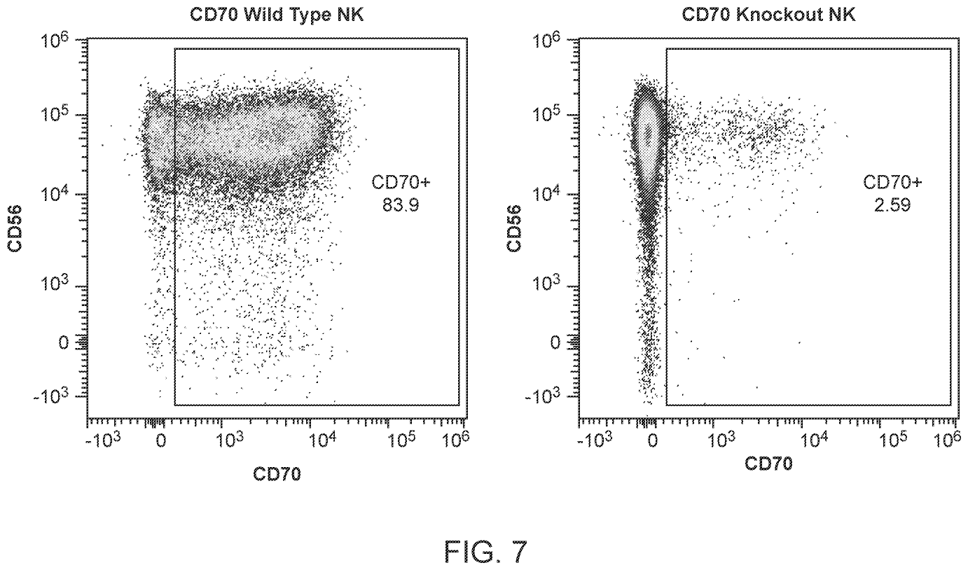

Activated NK cells were harvested at Day 8 post-activation, resuspended at 5×10⁷ cells/ml in Resuspension Buffer T (Thermo Fisher) and incubated with a pre-formed CD70crRNA-Cas9 complex (Integrated DNA Technologies). Cells were electroporated with the Neon Transfection System (ThermoFisher Scientific) using 2 pulses at 2000V and 10 ms pulse width. NK cells were recovered in warm NK MACS media (Miltenyi Biotec) containing 500 IU/mL IL-2. 48 hours later, NK cells were transferred to a G-REX flask (Wilson Wolf) with 1:1 ratio of irradiated artificial antigen presenting cells (aAPCs) and cultured in AIM-V media supplemented with 100 IU IL-2/mL for 4 days. Knockout of CD70 was assessed by flow cytometry on an Attune N×T Flow Cytometer. As shown in FIG. 7, CD70 was efficiently knocked out from peripheral blood NK cells.

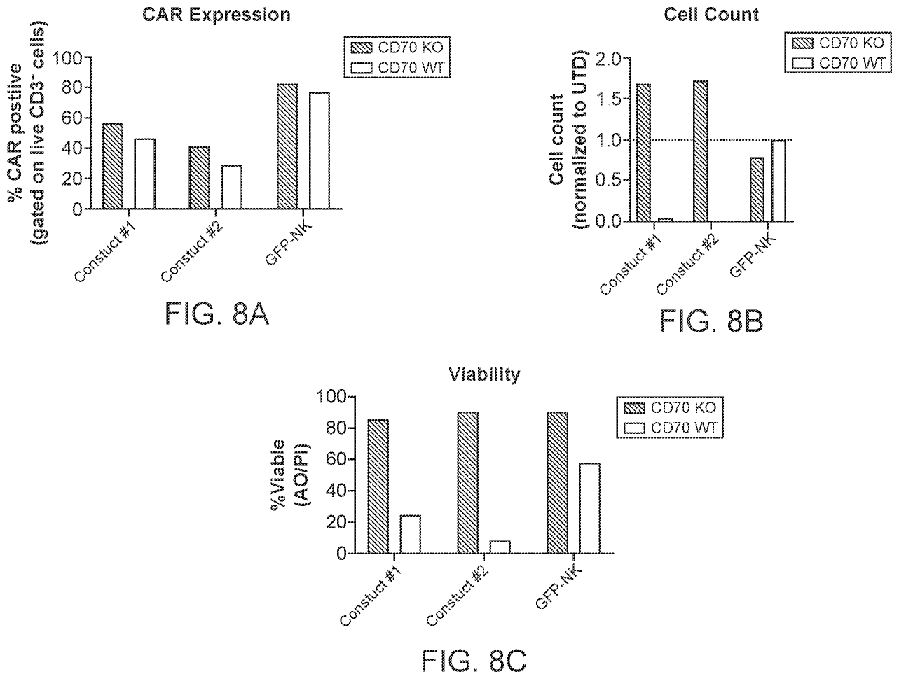

To examine the effect of CD70 knockout, 1×10⁶ of either wild-type or CD70 knock out NK cells were plated in a 24-well tissue culture plate in AIM-V media containing concentrated lentivirus particles. The cells were transduced to express either (a) a CAR comprising a CD27 extracellular domain, a CD27 transmembrane domain, a CD27 co-stimulatory domain, and a CD3z activation domain (Construct #1; SEQ ID NO: 643), (b) a CAR comprising an anti-CD70 scFv, a CD8α hinge, a CD8α transmembrane domain, a 4-1BB co-stimulatory domain, and a CD3z activation domain (Construct #2; SEQ ID NO: 2565), or (c) green fluorescent protein (GFP) ascontrol, untransduced cells were also used as control. Plates were spun at 1,000×g for 20 minutes at room temperature, and incubated at 37° C. 5% CO2 for 6 hours. Thereafter, the culture media was supplemented with IL-2 (100 IU/mL Bio-Techne) every other day. On Day 5 post-transduction, the cells were transferred to a 24 well G-REX plate (Wilson Wolf) and cultured for an additional 5 days in AIM-V media supplemented with IL-2. Cell counts and viability were assessed on Day 10 post-transduction utilizing acridine orange (AO)/propidium iodide (PI) staining on a Cellaca automated cell counter (Nexcelom Bioscience). Transduction efficiency was assessed by flow cytometry on Day 10 post-transduction. As shown in FIG. 8A, shows the expression of exemplary anti-CD70 CARs (i.e., Construct #1 or Construct #2) or GFP control was comparable between CD70 wild-type NK cells and CD70 knockout NK cells. Surprisingly, CD70 knockout NK cells expressing GFP exhibited greater cell expansion and viability as compared to CD70 wild-type NK cells expressing GFP (FIG. 8B and FIG. 8C). This effect was also observed in cells expressing each anti-CD70 CAR. The viability of CD70 wild-type NK cells expressing each CAR construct was less than or equal to 25% viable, while the viability of CD70 knockout NK cells expressing the same CAR constructs was above 85%. Additionally, CD70 wild-type NK cells expressing the GFP control were about 58% viable, while CD70 knockout NK cells expressing the same GFP control were about 90% viable (FIG. 8C).

Without wishing to be bound by theory, the observed difference in cell count among the NK cells expressing the CAR constructs may be due to reduced fratricide given that NK cells express high amounts of CD70 following activation.

### Effector NK Cells Expressing Anti-CD70 CARs Kill CD70 Wildtype NK Cells but not CD70 Knockout NK Cells

To determine whether NK cells expressing an anti-CD70 CAR construct were directly killing CD70-expressing NK cells, autologous CD70 wild-type (WT) NK cells and CD70 Knockout (KO) NK cells were labelled with CellTrace Violet dye (CTV) (ThermoFisher Scientific) and co-cultured at multiple effector to target cell (E/T) ratios with CD70 KO NK cells expressing either of the anti-CD70 CAR constructs (i.e., Construct #1 or Construct #2), or untransduced CD70 KO cells (UTD; asnegative control).

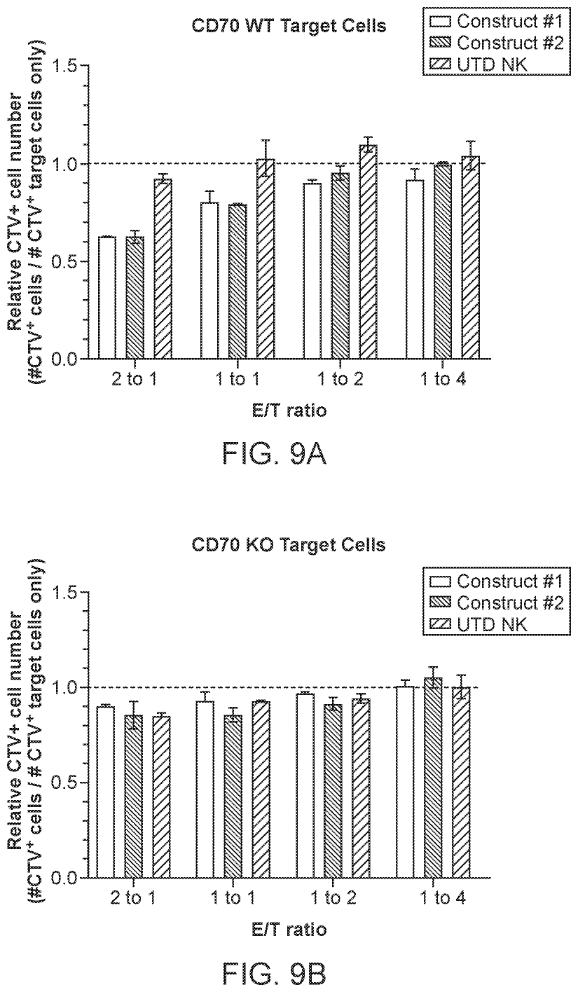

Briefly, the target cells were labelled with CTV according to the manufacturer's instructions, resuspended in media, and plated at 50,000 cells/well in a 96 well U-bottom plate (ThermoFisher Scientific). The effector cells (i.e., CD70 KO NK cells expressing either Construct #1 or Construct #2, or UTD control cells, were combined at E/T ratios of either 4:1, 2:1, 1:1, or 0.5:1. Cells were cultured for 4 hours at 37° C., 5% CO2, and stained with antibodies against CD56, CD16, and CD70. Remaining CTV+ target cells were enumerated by running a fixed volume of stained cells on an Attune N×T Flow Cytometer. The number of remaining CTV+ target cells per well was normalized against the number of CTV+ target cells in wells containing target cells only. As shown in FIG. 9A and FIG. 9B, increased target cell killing by effector CD70 knockout NK cells expressing either CAR was observed when target CD70 wild-type NK cells were used as compared to when target CD70 knockout NK cells were used indicating that NK cells expressing each CAR directly kill CD70-expressing NK cells.

### Effector NK Cells Expressing Anti-CD70 CARs are Capable of Killing the Target Acute Myeloid Leukemia MOLM-13 Cell Line in a CD70-Dependent Manner

To determine whether NK cells expressing an anti-CD70 CAR construct were capable of directly killing target tumor cells in a CD70-dependent manner, in vitro cytotoxicity assays were performed using the target tumor cell line MOM-13, an acute myeloid leukemia cell line modified to knock-out CD70. Briefly, MOLM-13 expressing luciferase and endogenous CD70 or MOLM-13 engineered to knockout CD70 and express luciferase were plated at 25,000 cells/well in a 96 well plate. Effector CD70 knockout NK cells expressing either of the anti-CD70 CAR constructs (i.e., Construct #1 or Construct #2), or untransduced NK cells (UTD NK; control) were co-cultured at either a 1:1 or 0.5 to 1 E/T ratio in a 37° C. 5% CO₂ incubator. After 4 hours, Steady-Glo Luciferase Assay Reagent (Promega) was added at a 1:1 volume to label, and the plates were placed on an orbital plate shaker rotating at 500 rpm for 5 minutes. Lysed cells were transferred to a black clear bottom plate, and the luciferase signal was determined on a GloMax Discover System plate reader (Promega). The percent killing activity was determined using the following formula:

$$( \frac{RLU ( target cells only ) - RLU ( target cells + NK cells )}{RLU ( target cells only )} ) * 100$$

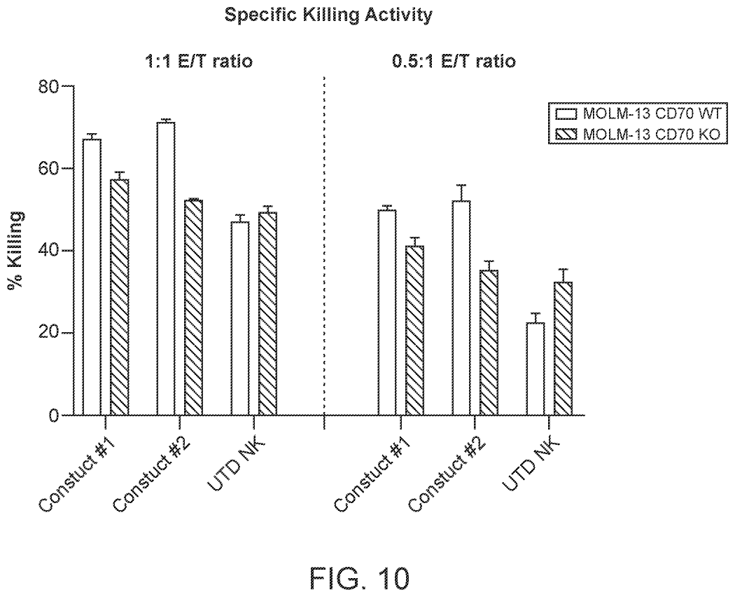

As shown in FIG. 10, CD70 knockout NK cells expressing anti-CD70 CARs (i.e., Construct #1 or Construct #2) exhibited increased cytotoxic activity against MOLM-13 target cells expressing endogenous CD70 as compared to MOLM-13 CD70 knockout target cells (e.g., at a 1:1 E/T ratio, 67% killing of MOLM-13 target cells expressing endogenous CD70 vs. 57% killing of MOLM-13 CD70 KO target cells by NK cells expressing the anti-CD70 CAR of Construct #1).

### Example 5—Anti-CD70 Car Transduction of Peripheral Blood NKC Cells was Inversely Correlated with CD70 Expression

Peripheral blood NK cells were isolated from human leukapherisis samples using immunomagnetic separation beads. Isolated NK cells were activated with mitomycin C-treated artificial antigen presenting cells (aAPCs) prior to transduction and 75 IU/mL of recombinant IL-2 to enable NK cell activation and expansion. About 1.5×10⁵ NK cells were plated in 96-well tissue culture plates in a volume of 150 μL of AIM-V media containing 200 IU/mL of recombinant IL-2 and varying volumes of concentrated lentivirus particles encoding either an anti-CD70 CAR comprising a CD27 extracellular domain (“anti-CD70 CAR (CD27 receptor)”) or ZsGreen fluorescent protein (“ZsGreen”; as control). Plates were spun at 900×g for 90 minutes at room temperature, and then incubated at 37° C. 5% CO₂ for 2 days. After 2 days, the transduced NK cells were transferred to new 96-well tissue culture plates with fresh media containing 200 IU/mL of recombinant human IL-2 and then incubated at 37° C., 5% CO₂ for an additional 2 days. Four days post-transduction, the NK cells were harvested and washed with a solution of Dulbecco's phosphate-buffered saline (dPBS) containing 2% fetal bovine serum.

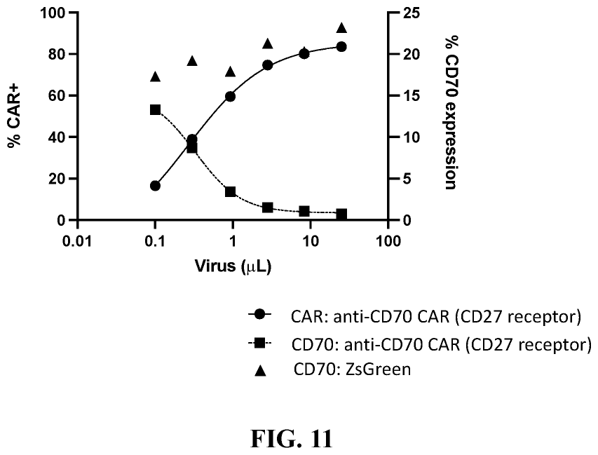

The NK cells were then incubated with a staining cocktail containing BRILLIANT VIOLET 421™ dye-conjugated anti-CD3 antibody (BIOLEGEND), BRILLIANT VIOLET 711™ dye-conjugated anti-CD16 antibody (BIOLEGEND), PE/DAZZLE™ 594 dye-conjugated anti-CD56 antibody (BIOLEGEND), allophycocyanin (APC)-conjugated anti-CD27 antibody (BIOLEGEND), phycoerythrin (PE)-conjugated anti-CD70 antibody (BIOLEGEND), and LIVE/DEAD™ fixable near-IR stain (THERMO FISHER SCIENTIFIC) for 30 minutes, then washed with a solution of dPBS containing 2% FBS. Samples were analyzed on the ATTUNE™ NXT flow cytometer (THERMO FISHER). Flow cytometry data were analyzed using FLOWJO™ software (BD BIOSCIENCES). CAR expression was measured as the percent of NK cells stained positively with the APC-conjugated anti-CD27 antibody, and CD70 expression was measured as the percent of NK cells stained positively with the PE-conjugated anti-CD70 antibody. The results are described in FIG. 11.

As shown in FIG. 11, CAR transduction and CD70 expression were found to be inversely correlated. Increasing expression of anti-CD70 CAR correlated with decreasing levels of CD70-positive NK cells. Thus, CD70 expression appears to drive anti-CD70 CAR-NK-mediated fratricide of CD70-expressing NK cells in vitro.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### EMBODIMENTS

Embodiment 1. A method of making a population of genetically engineered Natural Killer (NK) cells, the method comprising:

(a) providing a population of NK cells;

(b) contacting the population of NK cells with a CD70 inhibitor; and

(c) expanding the population of NK cells in vitro.

Embodiment 2. The method of embodiment 1, wherein the population of NK cells is a population of human NK cells.

Embodiment 3. The method of embodiment 1, wherein the population of NK cells exhibits at least about 25% greater cell expansion compared to a population of NK cells that is not contacted with the CD70 inhibitor.

Embodiment 4. The method of embodiment 1, wherein the population of NK cells exhibits at least about 30%, about 40%, about 50%, about 60%, about 70%, about 80%, about 90% or about 100% greater cell expansion compared to a population of NK cells that is expanded under the same conditions but is not contacted with the CD70 inhibitor.

Embodiment 5. The method of embodiments 1-4, further comprising isolating at least one of CD56⁺ cells and/or CD3⁻/CD56⁺ cells from a population of peripheral blood mononuclear cells (PBMCs) to obtain the population of NK cells.

Embodiment 6. The method of embodiments 1-5, wherein the population of NK cells is derived from umbilical cord blood cells, PBMCs, mobilized peripheral blood stem cells (PBSCs), unmobilized PBSCs, human embryonic stem cells (hESCs), induced pluripotent stem cells (iPSCs), mesenchymal stem cells (MSCs), hematopoietic stem cells (HSCs), bone marrow, or CD34⁺ cells.

Embodiment 7. The method of any one of embodiments 1-6, wherein the expanding comprises culturing the population of NK cells in the presence of feeder cells.

Embodiment 8. The method of embodiment 7, wherein the feeder cells are an immortalized cell line.

Embodiment 9. The method of embodiment 7, wherein the feeder cells are autologous feeder cells.

Embodiment 10. The method of any one of embodiments 7-9, wherein the feeder cells have been irradiated.

Embodiment 11. The method of any one of embodiments 1-10, wherein the expanding comprises culturing the population of NK cells in a culture medium comprising recombinant human IL-12, recombinant human IL-8 and/or recombinant human IL-21.

Embodiment 12. The method of any one of embodiments 1-11, wherein the expanding is performed from about 1 day to about 7 days.

Embodiment 13. The method of any one of embodiments 1-11, wherein the expanding is performed from about 8 days to about 14 days.

Embodiment 14. The method of any one of embodiments 1-11, wherein the expanding is performed from about 15 days to about 21 days.

Embodiment 15. The method of any one of embodiments 1-11, wherein the expanding is performed from about 22 days to about 28 days.

Embodiment 16. The method of any one of embodiments 1-11, wherein the expanding is performed from about 29 days to about 42 days.

Embodiment 17. The method of any one of embodiments 1-16, wherein the CD70 inhibitor decreases the expression of CD70 polypeptide in at least one NK cell of the population of NK cells.

Embodiment 18. The method of any one of embodiments 1-17 wherein the CD70 inhibitor ablates the expression of CD70 polypeptide in at least one NK cell of the population of NK cells.

Embodiment 19. The method of any one of embodiments 1-18, wherein the CD70 inhibitor comprises a small interfering RNA (siRNA) that targets CD70 mRNA, a short hairpin RNA (shRNA) that targets CD70 mRNA, a nucleic acid encoding a siRNA that targets CD70 mRNA, a nucleic acid encoding an shRNA that targets CD70 mRNA, or a combination of any of the foregoing.

Embodiment 20. The method of any one of embodiments 1-19, wherein the CD70 inhibitor comprises a shRNA that targets CD70 mRNA or a nucleic acid encoding a shRNA that targets CD70 mRNA.

Embodiment 21. The method of embodiment 20, wherein the shRNA that targets CD70 mRNA comprises the nucleic acid sequence of any one of SEQ ID NOs: 2647-2652.

Embodiment 22. The method of any one of embodiments 1-18, wherein the CD70 inhibitor comprises an RNA-guided endonuclease and a guide RNA (gRNA) targeting a CD70 gene.

Embodiment 23. The method of any one of embodiments 1-16, wherein the CD70 inhibitor decreases the cell surface level of CD70 polypeptide in at least one NK cell of the population of NK cells.

Embodiment 24. The method of any one of embodiments 1-16 and 23, wherein the CD70 inhibitor comprises a Protein Expression Blocker (PEBL) or a nucleic acid encoding a PEBL, wherein the PEBL comprises a first antigen recognition domain that specifically binds human CD70 and one or more of a localizing domain, an intracellular retention domain and an endoplasmic reticulum (ER) retention domain.

Embodiment 25. The method of embodiment 24, wherein the first antigen recognition domain comprises a heavy chain variable domain (VH) and a light chain variable domain (VL), wherein: (a) the VH comprises a heavy chain complementarity determining region 1 (CDRH1) of SEQ ID NO: 86, a heavy chain complementarity determining region 2 (CDRH2) of SEQ ID NO: 87, and a heavy chain complementarity determining region 3 (CDRH3) of SEQ ID NO: 88, and the VL comprises a light chain complementarity determining region 1 (CDRL1) of SEQ ID NO: 89, a light chain complementarity determining region 2 (CDRL2) of SEQ ID NO: 90, and a light chain complementarity determining region 3 (CDRL3) of SEQ ID NO: 91; (b) the VH comprises a CDRH1 of SEQ ID NO: 25, a CDRH2 of SEQ ID NO: 26, and a CDRH3 of SEQ ID NO: 27, and the VL comprises a CDRL1 of SEQ ID NO: 28, a CDRL2 of SEQ ID NO: 29, and a CDRL3 of SEQ ID NO: 30; (c) the VH comprises a CDRH1 of SEQ ID NO: 35, a CDRH2 of SEQ ID NO: 36, and a CDRH3 of SEQ ID NO: 37, and the VL comprises a CDRL1 of SEQ ID NO: 38, a CDRL2 of SEQ ID NO: 39, and a CDRL3 of SEQ ID NO: 40; (d) the VH comprises a CDRH1 of SEQ ID NO: 45, a CDRH2 of SEQ ID NO: 46, and a CDRH3 of SEQ ID NO: 47, and the VL comprises a CDRL1 of SEQ ID NO: 48, a CDRL2 of SEQ ID NO: 49, and a CDRL3 of SEQ ID NO: 50; (e) the VH comprises a CDRH1 of SEQ ID NO: 55, a CDRH2 of SEQ ID NO: 56, and a CDRH3 of SEQ ID NO: 57, and the VL comprises a CDRL1 of SEQ ID NO: 58, a CDRL2 of SEQ ID NO: 59, and a CDRL3 of SEQ ID NO: 60; (f) the VH comprises a CDRH1 of SEQ ID NO: 15, a CDRH2 of SEQ ID NO: 16, and a CDRH3 of SEQ ID NO: 17, and the VL comprises a CDRL1 of SEQ ID NO: 18, a CDRL2 of SEQ ID NO: 19, and a CDRL3 of SEQ ID NO: 20; (g) the VH comprises a CDRH1 of SEQ ID NO: 96, a CDRH2 of SEQ ID NO: 97, and a CDRH3 of SEQ ID NO: 98, and the VL comprises a CDRL1 of SEQ ID NO: 99, a CDRL2 of SEQ ID NO: 100, and a CDRL3 of SEQ ID NO: 101; (h) the VH comprises a CDRH1 of SEQ ID NO: 196, a CDRH2 of SEQ ID NO: 197, and a CDRH3 of SEQ ID NO: 198, and the VL comprises a CDRL1 of SEQ ID NO: 478, a CDRL2 of SEQ ID NO: 479, and a CDRL3 of SEQ ID NO: 480; (i) the VH comprises a CDRH1 of SEQ ID NO: 202, a CDRH2 of SEQ ID NO: 203, and a CDRH3 of SEQ ID NO: 204, and the VL comprises a CDRL1 of SEQ ID NO: 481, a CDRL2 of SEQ ID NO: 482, and a CDRL3 of SEQ ID NO: 483; (j) the VH comprises a CDRH1 of SEQ ID NO: 1170, a CDRH2 of SEQ ID NO: 1171, and a CDRH3 of SEQ ID NO: 1172, and the VL comprises a CDRL1 of SEQ ID NO: 1857, a CDRL2 of SEQ ID NO: 1858, and a CDRL3 of SEQ ID NO: 1859; (k) the VH comprises a CDRH1 of SEQ ID NO: 1173, a CDRH2 of SEQ ID NO: 1174, and a CDRH3 of SEQ ID NO: 1175, and the VL comprises a CDRL1 of SEQ ID NO: 1860, a CDRL2 of SEQ ID NO: 1861, and a CDRL3 of SEQ ID NO: 1862; (1) the VH comprises a CDRH1 of SEQ ID NO: 1176, a CDRH2 of SEQ ID NO: 1177, and a CDRH3 of SEQ ID NO: 1178, and the VL comprises a CDRL1 of SEQ ID NO: 1863, a CDRL2 of SEQ ID NO: 1864, and a CDRL3 of SEQ ID NO: 1865; (m) the VH comprises a CDRH1 of SEQ ID NO: 1179, a CDRH2 of SEQ ID NO: 1180, and a CDRH3 of SEQ ID NO: 1181, and the VL comprises a CDRL1 of SEQ ID NO: 1866, a CDRL2 of SEQ ID NO: 1867, and a CDRL3 of SEQ ID NO: 1868; (n) the VH comprises a CDRH1 of SEQ ID NO: 1182, a CDRH2 of SEQ ID NO: 1183, and a CDRH3 of SEQ ID NO: 1184, and the VL comprises a CDRL1 of SEQ ID NO: 1869, a CDRL2 of SEQ ID NO: 1870, and a CDRL3 of SEQ ID NO: 1871; (o) the VH comprises a CDRH1 of SEQ ID NO: 1185, a CDRH2 of SEQ ID NO: 1186, and a CDRH3 of SEQ ID NO: 1187, and the VL comprises a CDRL1 of SEQ ID NO: 1872, a CDRL2 of SEQ ID NO: 1873, and a CDRL3 of SEQ ID NO: 1874; (p) the VH comprises a CDRH1 of SEQ ID NO: 1188, a CDRH2 of SEQ ID NO: 1189, and a CDRH3 of SEQ ID NO: 1190, and the VL comprises a CDRL1 of SEQ ID NO: 1875, a CDRL2 of SEQ ID NO: 1876, and a CDRL3 of SEQ ID NO: 1877; (q) the VH comprises a CDRH1 of SEQ ID NO: 1524, a CDRH2 of SEQ ID NO: 1525, and a CDRH3 of SEQ ID NO: 1526, and the VL comprises a CDRL1 of SEQ ID NO: 2211, a CDRL2 of SEQ ID NO: 2212, and a CDRL3 of SEQ ID NO: 2213; (r) the VH comprises a CDRH1 of SEQ ID NO: 1527, a CDRH2 of SEQ ID NO: 1528, and a CDRH3 of SEQ ID NO: 1529, and the VL comprises a CDRL1 of SEQ ID NO: 2214, a CDRL2 of SEQ ID NO: 2215, and a CDRL3 of SEQ ID NO: 2216; (s) the VH comprises a CDRH1 of SEQ ID NO: 1530, a CDRH2 of SEQ ID NO: 1531, and a CDRH3 of SEQ ID NO: 1532, and the VL comprises a CDRL1 of SEQ ID NO: 2217, a CDRL2 of SEQ ID NO: 2218, and a CDRL3 of SEQ ID NO: 2219; (t) the VH comprises a CDRH1 of SEQ ID NO: 1533, a CDRH2 of SEQ ID NO: 1534, and a CDRH3 of SEQ ID NO: 1535, and the VL comprises a CDRL1 of SEQ ID NO: 2220, a CDRL2 of SEQ ID NO: 2221, and a CDRL3 of SEQ ID NO: 2222; (u) the VH comprises a CDRH1 of SEQ ID NO: 1539, a CDRH2 of SEQ ID NO: 1540, and a CDRH3 of SEQ ID NO: 1541, and the VL comprises a CDRL1 of SEQ ID NO: 2226, a CDRL2 of SEQ ID NO: 2227, and a CDRL3 of SEQ ID NO: 2228; (v) the VH comprises a CDRH1 of SEQ ID NO: 1542, a CDRH2 of SEQ ID NO: 1543, and a CDRH3 of SEQ ID NO: 1544, and the VL comprises a CDRL1 of SEQ ID NO: 2229, a CDRL2 of SEQ ID NO: 2230, and a CDRL3 of SEQ ID NO: 2231; (w) the VH comprises a CDRH1 of SEQ ID NO: 1548, a CDRH2 of SEQ ID NO: 1549, and a CDRH3 of SEQ ID NO: 1550, and the VL comprises a CDRL1 of SEQ ID NO: 2235, a CDRL2 of SEQ ID NO: 2236, and a CDRL3 of SEQ ID NO: 2237; (x) the VH comprises a CDRH1 of SEQ ID NO: 1551, a CDRH2 of SEQ ID NO: 1552, and a CDRH3 of SEQ ID NO: 1553, and the VL comprises a CDRL1 of SEQ ID NO: 2238, a CDRL2 of SEQ ID NO: 2239, and a CDRL3 of SEQ ID NO: 2240; (y) the VH comprises a CDRH1 of SEQ ID NO: 1554, a CDRH2 of SEQ ID NO: 1555, and a CDRH3 of SEQ ID NO: 1556, and the VL comprises a CDRL1 of SEQ ID NO: 2241, a CDRL2 of SEQ ID NO: 2242, and a CDRL3 of SEQ ID NO: 2243; (z) the VH comprises a CDRH1 of SEQ ID NO: 1557, a CDRH2 of SEQ ID NO: 1558, and a CDRH3 of SEQ ID NO: 1559, and the VL comprises a CDRL1 of SEQ ID NO: 2244, a CDRL2 of SEQ ID NO: 2245, and a CDRL3 of SEQ ID NO: 2246; (aa) the VH comprises a CDRH1 of SEQ ID NO: 1560, a CDRH2 of SEQ ID NO: 1561, and a CDRH3 of SEQ ID NO: 1562, and the VL comprises a CDRL1 of SEQ ID NO: 2247, a CDRL2 of SEQ ID NO: 2248, and a CDRL3 of SEQ ID NO: 2249; (bb) the VH comprises a CDRH1 of SEQ ID NO: 1563, a CDRH2 of SEQ ID NO: 1564, and a CDRH3 of SEQ ID NO: 1565, and the VL comprises a CDRL1 of SEQ ID NO: 2250, a CDRL2 of SEQ ID NO: 2251, and a CDRL3 of SEQ ID NO: 2252; (cc) the VH comprises a CDRH1 of SEQ ID NO: 1566, a CDRH2 of SEQ ID NO: 1567, and a CDRH3 of SEQ ID NO: 1568, and the VL comprises a CDRL1 of SEQ ID NO: 2253, a CDRL2 of SEQ ID NO: 2254, and a CDRL3 of SEQ ID NO: 2255; (dd) the VH comprises a CDRH1 of SEQ ID NO: 1572, a CDRH2 of SEQ ID NO: 1573, and a CDRH3 of SEQ ID NO: 1574, and the VL comprises a CDRL1 of SEQ ID NO: 2259, a CDRL2 of SEQ ID NO: 2260, and a CDRL3 of SEQ ID NO: 2261; (ee) the VH comprises a CDRH1 of SEQ ID NO: 1575, a CDRH2 of SEQ ID NO: 1576, and a CDRH3 of SEQ ID NO: 1577, and the VL comprises a CDRL1 of SEQ ID NO: 2262, a CDRL2 of SEQ ID NO: 2263, and a CDRL3 of SEQ ID NO: 2264; (ff) the VH comprises a CDRH1 of SEQ ID NO: 1578, a CDRH2 of SEQ ID NO: 1579, and a CDRH3 of SEQ ID NO: 1580, and the VL comprises a CDRL1 of SEQ ID NO: 2265, a CDRL2 of SEQ ID NO: 2266, and a CDRL3 of SEQ ID NO: 2267; (gg) the VH comprises a CDRH1 of SEQ ID NO: 1587, a CDRH2 of SEQ ID NO: 1588, and a CDRH3 of SEQ ID NO: 1589, and the VL comprises a CDRL1 of SEQ ID NO: 2274, a CDRL2 of SEQ ID NO: 2275, and a CDRL3 of SEQ ID NO: 2276; (hh) the VH comprises a CDRH1 of SEQ ID NO: 1590, a CDRH2 of SEQ ID NO: 1591, and a CDRH3 of SEQ ID NO: 1592, and the VL comprises a CDRL1 of SEQ ID NO: 2277, a CDRL2 of SEQ ID NO: 2278, and a CDRL3 of SEQ ID NO: 2279; (ii) the VH comprises a CDRH1 of SEQ ID NO: 1593, a CDRH2 of SEQ ID NO: 1594, and a CDRH3 of SEQ ID NO: 1595, and the VL comprises a CDRL1 of SEQ ID NO: 2280, a CDRL2 of SEQ ID NO: 2281, and a CDRL3 of SEQ ID NO: 2282; (jj) the VH comprises a CDRH1 of SEQ ID NO: 1596, a CDRH2 of SEQ ID NO: 1597, and a CDRH3 of SEQ ID NO: 1598, and the VL comprises a CDRL1 of SEQ ID NO: 2283, a CDRL2 of SEQ ID NO: 2284, and a CDRL3 of SEQ ID NO: 2285; (kk) the VH comprises a CDRH1 of SEQ ID NO: 1599, a CDRH2 of SEQ ID NO: 1560, and a CDRH3 of SEQ ID NO: 1561, and the VL comprises a CDRL1 of SEQ ID NO: 2286, a CDRL2 of SEQ ID NO: 2287, and a CDRL3 of SEQ ID NO: 2288; (11) the VH comprises a CDRH1 of SEQ ID NO: 1602, a CDRH2 of SEQ ID NO: 1603, and a CDRH3 of SEQ ID NO: 1604, and the VL comprises a CDRL1 of SEQ ID NO: 2289, a CDRL2 of SEQ ID NO: 2290, and a CDRL3 of SEQ ID NO: 2291; (mm) the VH comprises a CDRH1 of SEQ ID NO: 1605, a CDRH2 of SEQ ID NO: 1606, and a CDRH3 of SEQ ID NO: 1607, and the VL comprises a CDRL1 of SEQ ID NO: 2292, a CDRL2 of SEQ ID NO: 2293, and a CDRL3 of SEQ ID NO: 2294; (nn) the VH comprises a CDRH1 of SEQ ID NO: 1608, a CDRH2 of SEQ ID NO: 1609, and a CDRH3 of SEQ ID NO: 1610, and the VL comprises a CDRL1 of SEQ ID NO: 2295, a CDRL2 of SEQ ID NO: 2296, and a CDRL3 of SEQ ID NO: 2297; (oo) the VH comprises a CDRH1 of SEQ ID NO: 1611, a CDRH2 of SEQ ID NO: 1612, and a CDRH3 of SEQ ID NO: 1613, and the VL comprises a CDRL1 of SEQ ID NO: 2298, a CDRL2 of SEQ ID NO: 2299, and a CDRL3 of SEQ ID NO: 2300; (pp) the VH comprises a CDRH1 of SEQ ID NO: 1614, a CDRH2 of SEQ ID NO: 1615, and a CDRH3 of SEQ ID NO: 1616, and the VL comprises a CDRL1 of SEQ ID NO: 2301, a CDRL2 of SEQ ID NO: 2302, and a CDRL3 of SEQ ID NO: 2303; (qq) the VH comprises a CDRH1 of SEQ ID NO: 1617, a CDRH2 of SEQ ID NO: 1618, and a CDRH3 of SEQ ID NO: 1619, and the VL comprises a CDRL1 of SEQ ID NO: 2304, a CDRL2 of SEQ ID NO: 2305, and a CDRL3 of SEQ ID NO: 2306; (rr) the VH comprises a CDRH1 of SEQ ID NO: 1626, a CDRH2 of SEQ ID NO: 1627, and a CDRH3 of SEQ ID NO: 1628, and the VL comprises a CDRL1 of SEQ ID NO: 2313, a CDRL2 of SEQ ID NO: 2314, and a CDRL3 of SEQ ID NO: 2315; (ss) the VH comprises a CDRH1 of SEQ ID NO: 1629, a CDRH2 of SEQ ID NO: 1630, and a CDRH3 of SEQ ID NO: 1631, and the VL comprises a CDRL1 of SEQ ID NO: 2316, a CDRL2 of SEQ ID NO: 2317, and a CDRL3 of SEQ ID NO: 2318; (tt) the VH comprises a CDRH1 of SEQ ID NO: 1632, a CDRH2 of SEQ ID NO: 1633, and a CDRH3 of SEQ ID NO: 1634, and the VL comprises a CDRL1 of SEQ ID NO: 2319, a CDRL2 of SEQ ID NO: 2320, and a CDRL3 of SEQ ID NO: 2321; (uu) the VH comprises a CDRH1 of SEQ ID NO: 1635, a CDRH2 of SEQ ID NO: 1636, and a CDRH3 of SEQ ID NO: 1637, and the VL comprises a CDRL1 of SEQ ID NO: 2322, a CDRL2 of SEQ ID NO: 2323, and a CDRL3 of SEQ ID NO: 2324; (vv) the VH comprises a CDRH1 of SEQ ID NO: 1638, a CDRH2 of SEQ ID NO: 1639, and a CDRH3 of SEQ ID NO: 1640, and the VL comprises a CDRL1 of SEQ ID NO: 2325, a CDRL2 of SEQ ID NO: 2326, and a CDRL3 of SEQ ID NO: 2327; (ww) the VH comprises a CDRH1 of SEQ ID NO: 1641, a CDRH2 of SEQ ID NO: 1642, and a CDRH3 of SEQ ID NO: 1643, and the VL comprises a CDRL1 of SEQ ID NO: 2328, a CDRL2 of SEQ ID NO: 2329, and a CDRL3 of SEQ ID NO: 2330; (xx) the VH comprises a CDRH1 of SEQ ID NO: 1644, a CDRH2 of SEQ ID NO: 1645, and a CDRH3 of SEQ ID NO: 1646, and the VL comprises a CDRL1 of SEQ ID NO: 2331, a CDRL2 of SEQ ID NO: 2332, and a CDRL3 of SEQ ID NO: 2333; (yy) the VH comprises a CDRH1 of SEQ ID NO: 1647, a CDRH2 of SEQ ID NO: 1648, and a CDRH3 of SEQ ID NO: 1649, and the VL comprises a CDRL1 of SEQ ID NO: 2334, a CDRL2 of SEQ ID NO: 2335, and a CDRL3 of SEQ ID NO: 2336; (zz) the VH comprises a CDRH1 of SEQ ID NO: 1650, a CDRH2 of SEQ ID NO: 1651, and a CDRH3 of SEQ ID NO: 1652, and the VL comprises a CDRL1 of SEQ ID NO: 2337, a CDRL2 of SEQ ID NO: 2338, and a CDRL3 of SEQ ID NO: 2339; (aaa) the VH comprises a CDRH1 of SEQ ID NO: 1653, a CDRH2 of SEQ ID NO: 1654, and a CDRH3 of SEQ ID NO: 1655, and the VL comprises a CDRL1 of SEQ ID NO: 2340, a CDRL2 of SEQ ID NO: 2341, and a CDRL3 of SEQ ID NO: 2342; (bbb) the VH comprises a CDRH1 of SEQ ID NO: 1656, a CDRH2 of SEQ ID NO: 1657, and a CDRH3 of SEQ ID NO: 1658, and the VL comprises a CDRL1 of SEQ ID NO: 2343, a CDRL2 of SEQ ID NO: 2344, and a CDRL3 of SEQ ID NO: 2345; or (ccc) the VH comprises a CDRH1 of SEQ ID NO: 1659, a CDRH2 of SEQ ID NO: 1660, and a CDRH3 of SEQ ID NO: 1661, and the VL comprises a CDRL1 of SEQ ID NO: 2346, a CDRL2 of SEQ ID NO: 2347, and a CDRL3 of SEQ ID NO: 2348.

Embodiment 26. The method of embodiment 24 or 25, wherein the first antigen recognition domain comprises a VH and a VL, wherein: (a) the VH comprises SEQ ID NO: 82 and the VL comprises SEQ ID NO: 84; (b) the VH comprises SEQ ID NO: 21 and the VL comprises SEQ ID NO: 23; (c) the VH comprises SEQ ID NO: 31 and the VL comprises SEQ ID NO: 33; (d) the VH comprises SEQ ID NO: 41 and the VL comprises SEQ ID NO: 43; (e) the VH comprises SEQ ID NO: 51 and the VL comprises SEQ ID NO: 53; (f) the VH comprises SEQ ID NO: 61 and the VL comprises SEQ ID NO: 63; (g) the VH comprises SEQ ID NO: 693 and the VL comprises SEQ ID NO: 66; (h) the VH comprises SEQ ID NO: 694 and the VL comprises SEQ ID NO: 69; (i) the VH comprises SEQ ID NO: 695 and the VL comprises SEQ ID NO: 72; (j) the VH comprises SEQ ID NO: 74 and the VL comprises SEQ ID NO: 76; (k) the VH comprises SEQ ID NO: 78 and the VL comprises SEQ ID NO: 80; (1) the VH comprises SEQ ID NO: 11 and the VL comprises SEQ ID NO: 13; (m) the VH comprises SEQ ID NO: 92 and the VL comprises SEQ ID NO: 94; (n) the VH comprises SEQ ID NO: 102 and the VL comprises SEQ ID NO: 103; (o) the VH comprises SEQ ID NO: 104 and the VL comprises SEQ ID NO: 105; (p) the VH comprises SEQ ID NO: 712 and the VL comprises SEQ ID NO: 713; (q) the VH comprises SEQ ID NO: 714 and the VL comprises SEQ ID NO: 715; (r) the VH comprises SEQ ID NO: 716 and the VL comprises SEQ ID NO: 717; (s) the VH comprises SEQ ID NO: 718 and the VL comprises SEQ ID NO: 719; (t) the VH comprises SEQ ID NO: 720 and the VL comprises SEQ ID NO: 721; (u) the VH comprises SEQ ID NO: 722 and the VL comprises SEQ ID NO: 723; (v) the VH comprises SEQ ID NO: 724 and the VL comprises SEQ ID NO: 725; (w) the VH comprises SEQ ID NO: 948 and the VL comprises SEQ ID NO: 949; (x) the VH comprises SEQ ID NO: 950 and the VL comprises SEQ ID NO: 951; (y) the VH comprises SEQ ID NO: 952 and the VL comprises SEQ ID NO: 953; (z) the VH comprises SEQ ID NO: 954 and the VL comprises SEQ ID NO: 955; (aa) the VH comprises SEQ ID NO: 958 and the VL comprises SEQ ID NO: 959; (bb) the VH comprises SEQ ID NO: 960 and the VL comprises SEQ ID NO: 961; (cc) the VH comprises SEQ ID NO: 964 and the VL comprises SEQ ID NO: 965; (dd) the VH comprises SEQ ID NO: 966 and the VL comprises SEQ ID NO: 967; (ee) the VH comprises SEQ ID NO: 968 and the VL comprises SEQ ID NO: 969; (ff) the VH comprises SEQ ID NO: 970 and the VL comprises SEQ ID NO: 971; (gg) the VH comprises SEQ ID NO: 972 and the VL comprises SEQ ID NO: 973; (hh) the VH comprises SEQ ID NO: 974 and the VL comprises SEQ ID NO: 975; (ii) the VH comprises SEQ ID NO: 976 and the VL comprises SEQ ID NO: 977; (jj) the VH comprises SEQ ID NO: 980 and the VL comprises SEQ ID NO: 981; (kk) the VH comprises SEQ ID NO: 982 and the VL comprises SEQ ID NO: 983; (11) the VH comprises SEQ ID NO: 984 and the VL comprises SEQ ID NO: 985; (mm) the VH comprises SEQ ID NO: 990 and the VL comprises SEQ ID NO: 991; (nn) the VH comprises SEQ ID NO: 992 and the VL comprises SEQ ID NO: 993; (oo) the VH comprises SEQ ID NO: 994 and the VL comprises SEQ ID NO: 995; (pp) the VH comprises SEQ ID NO: 996 and the VL comprises SEQ ID NO: 997; (qq) the VH comprises SEQ ID NO: 998 and the VL comprises SEQ ID NO: 999; (rr) the VH comprises SEQ ID NO: 1000 and the VL comprises SEQ ID NO: 1001; (ss) the VH comprises SEQ ID NO: 1002 and the VL comprises SEQ ID NO: 1003; (tt) the VH comprises SEQ ID NO: 1004 and the VL comprises SEQ ID NO: 1005; (uu) the VH comprises SEQ ID NO: 1006 and the VL comprises SEQ ID NO: 1007; (vv) the VH comprises SEQ ID NO: 1008 and the VL comprises SEQ ID NO: 1009; (ww) the VH comprises SEQ ID NO: 1010 and the VL comprises SEQ ID NO: 1011; (xx) the VH comprises SEQ ID NO: 1016 and the VL comprises SEQ ID NO: 1017; (yy) the VH comprises SEQ ID NO: 1018 and the VL comprises SEQ ID NO: 1019; (zz) the VH comprises SEQ ID NO: 1020 and the VL comprises SEQ ID NO: 1021; (aaa) the VH comprises SEQ ID NO: 1022 and the VL comprises SEQ ID NO: 1023; (bbb) the VH comprises SEQ ID NO: 1024 and the VL comprises SEQ ID NO: 1025; (ccc) the VH comprises SEQ ID NO: 1026 and the VL comprises SEQ ID NO: 1027; (ddd) the VH comprises SEQ ID NO: 1028 and the VL comprises SEQ ID NO: 1029; (eee) the VH comprises SEQ ID NO: 1030 and the VL comprises SEQ ID NO: 1031; (fff) the VH comprises SEQ ID NO: 1032 and the VL comprises SEQ ID NO: 1033; (ggg) the VH comprises SEQ ID NO: 1034 and the VL comprises SEQ ID NO: 1035; (hhh) the VH comprises SEQ ID NO: 1036 and the VL comprises SEQ ID NO: 1037; or (iii) the VH comprises SEQ ID NO: 1038 and the VL comprises SEQ ID NO: 1039.

Embodiment 27. The method of any one of embodiments 1-16, wherein the CD70 inhibitor comprises an antagonistic anti-CD70 antibody or an antigen-binding fragment thereof.

Embodiment 28. The method of embodiment 27, wherein the antagonistic anti-CD70 antibody or the antigen-binding fragment thereof inhibits the interaction between CD70 and CD27.

Embodiment 29. The method of embodiment 27 or 28, wherein the antagonistic anti-CD70 antibody or the antigen-binding fragment thereof comprises a VH and a VL wherein: a) the VH comprises SEQ ID NO: 1162 and the VL comprises SEQ ID NO: 1163; b) the VH comprises SEQ ID NO: 51 and the VL comprises SEQ ID NO: 53; c) the VH comprises SEQ ID NO: 11 and the VL comprises SEQ ID NO: 13; d) the VH comprises SEQ ID NO: 694 and the VL comprises SEQ ID NO: 69; e) the VH comprises SEQ ID NO: 1118 and the VL comprises SEQ ID NO: 1119; f) the VH comprises SEQ ID NO: 1120 and the VL comprises SEQ ID NO: 1121; g) the VH comprises SEQ ID NO: 1116 and the VL comprises SEQ ID NO: 1117; h) the VH comprises SEQ ID NO: 1104 and the VL comprises SEQ ID NO: 1105; i) the VH comprises SEQ ID NO: 1094 and the VL comprises SEQ ID NO: 1095; j) the VH comprises SEQ ID NO: 1084 and the VL comprises SEQ ID NO: 1085; k) the VH comprises SEQ ID NO: 1092 and the VL comprises SEQ ID NO: 1093; 1) the VH comprises SEQ ID NO: 1082 and the VL comprises SEQ ID NO: 1083; or m) the VH comprises SEQ ID NO: 1074 and the VL comprises SEQ ID NO: 1075.

Embodiment 30. The method of any one of embodiments 27-29, wherein the antagonistic anti-CD70 antibody is cusatuzumab, MDX-1411, 27B3, 57B6, 59D10, 19G10, 9B2, 5B2, 9G2, 5F4, 9D1, and/or SGN70.

Embodiment 31. The method of any one of embodiments 1-30, wherein at least one cell in the population of NK cells comprises a knockout or knockdown of a cellular gene.

Embodiment 32. The method of embodiment 31, wherein the cellular gene is selected from CISH, PD1, TGFBR1, TGFBR2, or a combination thereof.

Embodiment 33. The method of any one of embodiments 1-32, further comprising (d) contacting the population of NK cells with a first polynucleotide encoding a first chimeric antigen receptor (CAR) under conditions sufficient to transfer the polynucleotide across a cell membrane of at least one NK cell in the population of NK cells, wherein the first CAR comprises: (i) an extracellular domain comprising a second antigen recognition domain that specifically binds human CD70; (ii) a transmembrane domain; and (iii) an intracellular domain.

Embodiment 34. The method of embodiment 33, further comprising culturing the modified population of NK cells under conditions suitable for integration of the first polynucleotide into the genome of at least one NK cell in the population of NK cells.

Embodiment 35. The method of embodiment 33 or 34, wherein step (d) is performed prior to step (b).

Embodiment 36. The method of embodiment 33 or 34, wherein step (d) is performed concurrently with step (b).

Embodiment 37. The method of embodiment 33 or 34, wherein step (d) is performed after step (b).

Embodiment 38. The method of embodiment 33 or 34, wherein step (d) is performed after step (c).

Embodiment 39. The method of embodiment 38, further comprising expanding the population of NK cells in vitro after step (d).

Embodiment 40. The method of any one of embodiments 1-39, wherein step (c) comprises expanding the population of NK cells by at least 1,000-fold in culture.

Embodiment 41. The method of any one of embodiments 1-40, wherein step (b) and/or step (d) comprises use of a viral vector, electroporation, a transposon/transposase system, a lipid nanoparticle or a charge-altering releasable transporter.

Embodiment 42. The method of embodiment 39, wherein step (b) and/or step (d) comprises the use of a viral vector, and wherein the viral vector is a lentivirus, a gamma retrovirus, an adeno-associated virus, an adenovirus, or a herpes simplex virus.

Embodiment 43. The method of embodiment 39, wherein step (b) and/or step (d) comprises the use of the transposon/transposase system, and wherein the transposon/transposase system comprises piggyBac, hyperactive piggyBac, Sleeping Beauty (SB), hyperactive SB, SB11, SB110, Tn7, TcBuster, hyperactive TcBuster, Mos1, Tcl/mariner, Tol2, mini-Tol2, Tc3, MuA, Himar I, Frog Prince, Helitron, L1 retrotransposon, IS5, Tn10, Tn903, SPIN, hAT, Hermes, hobo, AeBuster1, AeBuster2, AeBuster3, BtBuster1, BtBuster2, CfBuster1, or CfBuster2.

Embodiment 44. The method of any one of embodiments 33-43, wherein the first CAR comprises a signal peptide.

Embodiment 45. The method of any one of embodiments 33-44, wherein the second antigen recognition domain comprises a scFv comprising a VH and a VL, wherein: (a) the VH comprises a CDRH1 of SEQ ID NO: 86, a CDRH2 of SEQ ID NO: 87, and a CDRH3 of SEQ ID NO: 88, and the VL comprises a CDRL1 of SEQ ID NO: 89, a CDRL2 of SEQ ID NO: 90, and a CDRL3 of SEQ ID NO: 91; (b) the VH comprises a CDRH1 of SEQ ID NO: 25, a CDRH2 of SEQ ID NO: 26, and a CDRH3 of SEQ ID NO: 27, and the VL comprises a CDRL1 of SEQ ID NO: 28, a CDRL2 of SEQ ID NO: 29, and a CDRL3 of SEQ ID NO: 30; (c) the VH comprises a CDRH1 of SEQ ID NO: 35, a CDRH2 of SEQ ID NO: 36, and a CDRH3 of SEQ ID NO: 37, and the VL comprises a CDRL1 of SEQ ID NO: 38, a CDRL2 of SEQ ID NO: 39, and a CDRL3 of SEQ ID NO: 40; (d) the VH comprises a CDRH1 of SEQ ID NO: 45, a CDRH2 of SEQ ID NO: 46, and a CDRH3 of SEQ ID NO: 47, and the VL comprises a CDRL1 of SEQ ID NO: 48, a CDRL2 of SEQ ID NO: 49, and a CDRL3 of SEQ ID NO: 50; (e) the VH comprises a CDRH1 of SEQ ID NO: 55, a CDRH2 of SEQ ID NO: 56, and a CDRH3 of SEQ ID NO: 57, and the VL comprises a CDRL1 of SEQ ID NO: 58, a CDRL2 of SEQ ID NO: 59, and a CDRL3 of SEQ ID NO: 60; (f) the VH comprises a CDRH1 of SEQ ID NO: 15, a CDRH2 of SEQ ID NO: 16, and a CDRH3 of SEQ ID NO: 17, and the VL comprises a CDRL1 of SEQ ID NO: 18, a CDRL2 of SEQ ID NO: 19, and a CDRL3 of SEQ ID NO: 20; (g) the VH comprises a CDRH1 of SEQ ID NO: 96, a CDRH2 of SEQ ID NO: 97, and a CDRH3 of SEQ ID NO: 98, and the VL comprises a CDRL1 of SEQ ID NO: 99, a CDRL2 of SEQ ID NO: 100, and a CDRL3 of SEQ ID NO: 101; (h) the VH comprises a CDRH1 of SEQ ID NO: 196, a CDRH2 of SEQ ID NO: 197, and a CDRH3 of SEQ ID NO: 198, and the VL comprises a CDRL1 of SEQ ID NO: 478, a CDRL2 of SEQ ID NO: 479, and a CDRL3 of SEQ ID NO: 480; (i) the VH comprises a CDRH1 of SEQ ID NO: 202, a CDRH2 of SEQ ID NO: 203, and a CDRH3 of SEQ ID NO: 204, and the VL comprises a CDRL1 of SEQ ID NO: 481, a CDRL2 of SEQ ID NO: 482, and a CDRL3 of SEQ ID NO: 483; (j) the VH comprises a CDRH1 of SEQ ID NO: 1170, a CDRH2 of SEQ ID NO: 1171, and a CDRH3 of SEQ ID NO: 1172, and the VL comprises a CDRL1 of SEQ ID NO: 1857, a CDRL2 of SEQ ID NO: 1858, and a CDRL3 of SEQ ID NO: 1859; (k) the VH comprises a CDRH1 of SEQ ID NO: 1173, a CDRH2 of SEQ ID NO: 1174, and a CDRH3 of SEQ ID NO: 1175, and the VL comprises a CDRL1 of SEQ ID NO: 1860, a CDRL2 of SEQ ID NO: 1861, and a CDRL3 of SEQ ID NO: 1862; (1) the VH comprises a CDRH1 of SEQ ID NO: 1176, a CDRH2 of SEQ ID NO: 1177, and a CDRH3 of SEQ ID NO: 1178, and the VL comprises a CDRL1 of SEQ ID NO: 1863, a CDRL2 of SEQ ID NO: 1864, and a CDRL3 of SEQ ID NO: 1865; (m) the VH comprises a CDRH1 of SEQ ID NO: 1179, a CDRH2 of SEQ ID NO: 1180, and a CDRH3 of SEQ ID NO: 1181, and the VL comprises a CDRL1 of SEQ ID NO: 1866, a CDRL2 of SEQ ID NO: 1867, and a CDRL3 of SEQ ID NO: 1868; (n) the VH comprises a CDRH1 of SEQ ID NO: 1182, a CDRH2 of SEQ ID NO: 1183, and a CDRH3 of SEQ ID NO: 1184, and the VL comprises a CDRL1 of SEQ ID NO: 1869, a CDRL2 of SEQ ID NO: 1870, and a CDRL3 of SEQ ID NO: 1871; (o) the VH comprises a CDRH1 of SEQ ID NO: 1185, a CDRH2 of SEQ ID NO: 1186, and a CDRH3 of SEQ ID NO: 1187, and the VL comprises a CDRL1 of SEQ ID NO: 1872, a CDRL2 of SEQ ID NO: 1873, and a CDRL3 of SEQ ID NO: 1874; (p) the VH comprises a CDRH1 of SEQ ID NO: 1188, a CDRH2 of SEQ ID NO: 1189, and a CDRH3 of SEQ ID NO: 1190, and the VL comprises a CDRL1 of SEQ ID NO: 1875, a CDRL2 of SEQ ID NO: 1876, and a CDRL3 of SEQ ID NO: 1877; (q) the VH comprises a CDRH1 of SEQ ID NO: 1524, a CDRH2 of SEQ ID NO: 1525, and a CDRH3 of SEQ ID NO: 1526, and the VL comprises a CDRL1 of SEQ ID NO: 2211, a CDRL2 of SEQ ID NO: 2212, and a CDRL3 of SEQ ID NO: 2213; (r) the VH comprises a CDRH1 of SEQ ID NO: 1527, a CDRH2 of SEQ ID NO: 1528, and a CDRH3 of SEQ ID NO: 1529, and the VL comprises a CDRL1 of SEQ ID NO: 2214, a CDRL2 of SEQ ID NO: 2215, and a CDRL3 of SEQ ID NO: 2216; (s) the VH comprises a CDRH1 of SEQ ID NO: 1530, a CDRH2 of SEQ ID NO: 1531, and a CDRH3 of SEQ ID NO: 1532, and the VL comprises a CDRL1 of SEQ ID NO: 2217, a CDRL2 of SEQ ID NO: 2218, and a CDRL3 of SEQ ID NO: 2219; (t) the VH comprises a CDRH1 of SEQ ID NO: 1533, a CDRH2 of SEQ ID NO: 1534, and a CDRH3 of SEQ ID NO: 1535, and the VL comprises a CDRL1 of SEQ ID NO: 2220, a CDRL2 of SEQ ID NO: 2221, and a CDRL3 of SEQ ID NO: 2222; (u) the VH comprises a CDRH1 of SEQ ID NO: 1539, a CDRH2 of SEQ ID NO: 1540, and a CDRH3 of SEQ ID NO: 1541, and the VL comprises a CDRL1 of SEQ ID NO: 2226, a CDRL2 of SEQ ID NO: 2227, and a CDRL3 of SEQ ID NO: 2228; (v) the VH comprises a CDRH1 of SEQ ID NO: 1542, a CDRH2 of SEQ ID NO: 1543, and a CDRH3 of SEQ ID NO: 1544, and the VL comprises a CDRL1 of SEQ ID NO: 2229, a CDRL2 of SEQ ID NO: 2230, and a CDRL3 of SEQ ID NO: 2231; (w) the VH comprises a CDRH1 of SEQ ID NO: 1548, a CDRH2 of SEQ ID NO: 1549, and a CDRH3 of SEQ ID NO: 1550, and the VL comprises a CDRL1 of SEQ ID NO: 2235, a CDRL2 of SEQ ID NO: 2236, and a CDRL3 of SEQ ID NO: 2237; (x) the VH comprises a CDRH1 of SEQ ID NO: 1551, a CDRH2 of SEQ ID NO: 1552, and a CDRH3 of SEQ ID NO: 1553, and the VL comprises a CDRL1 of SEQ ID NO: 2238, a CDRL2 of SEQ ID NO: 2239, and a CDRL3 of SEQ ID NO: 2240; (y) the VH comprises a CDRH1 of SEQ ID NO: 1554, a CDRH2 of SEQ ID NO: 1555, and a CDRH3 of SEQ ID NO: 1556, and the VL comprises a CDRL1 of SEQ ID NO: 2241, a CDRL2 of SEQ ID NO: 2242, and a CDRL3 of SEQ ID NO: 2243; (z) the VH comprises a CDRH1 of SEQ ID NO: 1557, a CDRH2 of SEQ ID NO: 1558, and a CDRH3 of SEQ ID NO: 1559, and the VL comprises a CDRL1 of SEQ ID NO: 2244, a CDRL2 of SEQ ID NO: 2245, and a CDRL3 of SEQ ID NO: 2246; (aa) the VH comprises a CDRH1 of SEQ ID NO: 1560, a CDRH2 of SEQ ID NO: 1561, and a CDRH3 of SEQ ID NO: 1562, and the VL comprises a CDRL1 of SEQ ID NO: 2247, a CDRL2 of SEQ ID NO: 2248, and a CDRL3 of SEQ ID NO: 2249; (bb) the VH comprises a CDRH1 of SEQ ID NO: 1563, a CDRH2 of SEQ ID NO: 1564, and a CDRH3 of SEQ ID NO: 1565, and the VL comprises a CDRL1 of SEQ ID NO: 2250, a CDRL2 of SEQ ID NO: 2251, and a CDRL3 of SEQ ID NO: 2252; (cc) the VH comprises a CDRH1 of SEQ ID NO: 1566, a CDRH2 of SEQ ID NO: 1567, and a CDRH3 of SEQ ID NO: 1568, and the VL comprises a CDRL1 of SEQ ID NO: 2253, a CDRL2 of SEQ ID NO: 2254, and a CDRL3 of SEQ ID NO: 2255; (dd) the VH comprises a CDRH1 of SEQ ID NO: 1572, a CDRH2 of SEQ ID NO: 1573, and a CDRH3 of SEQ ID NO: 1574, and the VL comprises a CDRL1 of SEQ ID NO: 2259, a CDRL2 of SEQ ID NO: 2260, and a CDRL3 of SEQ ID NO: 2261; (ee) the VH comprises a CDRH1 of SEQ ID NO: 1575, a CDRH2 of SEQ ID NO: 1576, and a CDRH3 of SEQ ID NO: 1577, and the VL comprises a CDRL1 of SEQ ID NO: 2262, a CDRL2 of SEQ ID NO: 2263, and a CDRL3 of SEQ ID NO: 2264; (ff) the VH comprises a CDRH1 of SEQ ID NO: 1578, a CDRH2 of SEQ ID NO: 1579, and a CDRH3 of SEQ ID NO: 1580, and the VL comprises a CDRL1 of SEQ ID NO: 2265, a CDRL2 of SEQ ID NO: 2266, and a CDRL3 of SEQ ID NO: 2267; (gg) the VH comprises a CDRH1 of SEQ ID NO: 1587, a CDRH2 of SEQ ID NO: 1588, and a CDRH3 of SEQ ID NO: 1589, and the VL comprises a CDRL1 of SEQ ID NO: 2274, a CDRL2 of SEQ ID NO: 2275, and a CDRL3 of SEQ ID NO: 2276; (hh) the VH comprises a CDRH1 of SEQ ID NO: 1590, a CDRH2 of SEQ ID NO: 1591, and a CDRH3 of SEQ ID NO: 1592, and the VL comprises a CDRL1 of SEQ ID NO: 2277, a CDRL2 of SEQ ID NO: 2278, and a CDRL3 of SEQ ID NO: 2279; (ii) the VH comprises a CDRH1 of SEQ ID NO: 1593, a CDRH2 of SEQ ID NO: 1594, and a CDRH3 of SEQ ID NO: 1595, and the VL comprises a CDRL1 of SEQ ID NO: 2280, a CDRL2 of SEQ ID NO: 2281, and a CDRL3 of SEQ ID NO: 2282; (jj) the VH comprises a CDRH1 of SEQ ID NO: 1596, a CDRH2 of SEQ ID NO: 1597, and a CDRH3 of SEQ ID NO: 1598, and the VL comprises a CDRL1 of SEQ ID NO: 2283, a CDRL2 of SEQ ID NO: 2284, and a CDRL3 of SEQ ID NO: 2285; (kk) the VH comprises a CDRH1 of SEQ ID NO: 1599, a CDRH2 of SEQ ID NO: 1560, and a CDRH3 of SEQ ID NO: 1561, and the VL comprises a CDRL1 of SEQ ID NO: 2286, a CDRL2 of SEQ ID NO: 2287, and a CDRL3 of SEQ ID NO: 2288; (11) the VH comprises a CDRH1 of SEQ ID NO: 1602, a CDRH2 of SEQ ID NO: 1603, and a CDRH3 of SEQ ID NO: 1604, and the VL comprises a CDRL1 of SEQ ID NO: 2289, a CDRL2 of SEQ ID NO: 2290, and a CDRL3 of SEQ ID NO: 2291; (mm) the VH comprises a CDRH1 of SEQ ID NO: 1605, a CDRH2 of SEQ ID NO: 1606, and a CDRH3 of SEQ ID NO: 1607, and the VL comprises a CDRL1 of SEQ ID NO: 2292, a CDRL2 of SEQ ID NO: 2293, and a CDRL3 of SEQ ID NO: 2294; (nn) the VH comprises a CDRH1 of SEQ ID NO: 1608, a CDRH2 of SEQ ID NO: 1609, and a CDRH3 of SEQ ID NO: 1610, and the VL comprises a CDRL1 of SEQ ID NO: 2295, a CDRL2 of SEQ ID NO: 2296, and a CDRL3 of SEQ ID NO: 2297; (oo) the VH comprises a CDRH1 of SEQ ID NO: 1611, a CDRH2 of SEQ ID NO: 1612, and a CDRH3 of SEQ ID NO: 1613, and the VL comprises a CDRL1 of SEQ ID NO: 2298, a CDRL2 of SEQ ID NO: 2299, and a CDRL3 of SEQ ID NO: 2300; (pp) the VH comprises a CDRH1 of SEQ ID NO: 1614, a CDRH2 of SEQ ID NO: 1615, and a CDRH3 of SEQ ID NO: 1616, and the VL comprises a CDRL1 of SEQ ID NO: 2301, a CDRL2 of SEQ ID NO: 2302, and a CDRL3 of SEQ ID NO: 2303; (qq) the VH comprises a CDRH1 of SEQ ID NO: 1617, a CDRH2 of SEQ ID NO: 1618, and a CDRH3 of SEQ ID NO: 1619, and the VL comprises a CDRL1 of SEQ ID NO: 2304, a CDRL2 of SEQ ID NO: 2305, and a CDRL3 of SEQ ID NO: 2306; (rr) the VH comprises a CDRH1 of SEQ ID NO: 1626, a CDRH2 of SEQ ID NO: 1627, and a CDRH3 of SEQ ID NO: 1628, and the VL comprises a CDRL1 of SEQ ID NO: 2313, a CDRL2 of SEQ ID NO: 2314, and a CDRL3 of SEQ ID NO: 2315; (ss) the VH comprises a CDRH1 of SEQ ID NO: 1629, a CDRH2 of SEQ ID NO: 1630, and a CDRH3 of SEQ ID NO: 1631, and the VL comprises a CDRL1 of SEQ ID NO: 2316, a CDRL2 of SEQ ID NO: 2317, and a CDRL3 of SEQ ID NO: 2318; (tt) the VH comprises a CDRH1 of SEQ ID NO: 1632, a CDRH2 of SEQ ID NO: 1633, and a CDRH3 of SEQ ID NO: 1634, and the VL comprises a CDRL1 of SEQ ID NO: 2319, a CDRL2 of SEQ ID NO: 2320, and a CDRL3 of SEQ ID NO: 2321; (uu) the VH comprises a CDRH1 of SEQ ID NO: 1635, a CDRH2 of SEQ ID NO: 1636, and a CDRH3 of SEQ ID NO: 1637, and the VL comprises a CDRL1 of SEQ ID NO: 2322, a CDRL2 of SEQ ID NO: 2323, and a CDRL3 of SEQ ID NO: 2324; (vv) the VH comprises a CDRH1 of SEQ ID NO: 1638, a CDRH2 of SEQ ID NO: 1639, and a CDRH3 of SEQ ID NO: 1640, and the VL comprises a CDRL1 of SEQ ID NO: 2325, a CDRL2 of SEQ ID NO: 2326, and a CDRL3 of SEQ ID NO: 2327; (ww) the VH comprises a CDRH1 of SEQ ID NO: 1641, a CDRH2 of SEQ ID NO: 1642, and a CDRH3 of SEQ ID NO: 1643, and the VL comprises a CDRL1 of SEQ ID NO: 2328, a CDRL2 of SEQ ID NO: 2329, and a CDRL3 of SEQ ID NO: 2330; (xx) the VH comprises a CDRH1 of SEQ ID NO: 1644, a CDRH2 of SEQ ID NO: 1645, and a CDRH3 of SEQ ID NO: 1646, and the VL comprises a CDRL1 of SEQ ID NO: 2331, a CDRL2 of SEQ ID NO: 2332, and a CDRL3 of SEQ ID NO: 2333; (yy) the VH comprises a CDRH1 of SEQ ID NO: 1647, a CDRH2 of SEQ ID NO: 1648, and a CDRH3 of SEQ ID NO: 1649, and the VL comprises a CDRL1 of SEQ ID NO: 2334, a CDRL2 of SEQ ID NO: 2335, and a CDRL3 of SEQ ID NO: 2336; (zz) the VH comprises a CDRH1 of SEQ ID NO: 1650, a CDRH2 of SEQ ID NO: 1651, and a CDRH3 of SEQ ID NO: 1652, and the VL comprises a CDRL1 of SEQ ID NO: 2337, a CDRL2 of SEQ ID NO: 2338, and a CDRL3 of SEQ ID NO: 2339; (aaa) the VH comprises a CDRH1 of SEQ ID NO: 1653, a CDRH2 of SEQ ID NO: 1654, and a CDRH3 of SEQ ID NO: 1655, and the VL comprises a CDRL1 of SEQ ID NO: 2340, a CDRL2 of SEQ ID NO: 2341, and a CDRL3 of SEQ ID NO: 2342; (bbb) the VH comprises a CDRH1 of SEQ ID NO: 1656, a CDRH2 of SEQ ID NO: 1657, and a CDRH3 of SEQ ID NO: 1658, and the VL comprises a CDRL1 of SEQ ID NO: 2343, a CDRL2 of SEQ ID NO: 2344, and a CDRL3 of SEQ ID NO: 2345; or (ccc) the VH comprises a CDRH1 of SEQ ID NO: 1659, a CDRH2 of SEQ ID NO: 1660, and a CDRH3 of SEQ ID NO: 1661, and the VL comprises a CDRL1 of SEQ ID NO: 2346, a CDRL2 of SEQ ID NO: 2347, and a CDRL3 of SEQ ID NO: 2348.

Embodiment 46. The method of any one of embodiments 33-45, wherein the wherein the second antigen recognition domain comprises a scFv comprising a VH and a VL, wherein: (a) the VH comprises SEQ ID NO: 82 and the VL comprises SEQ ID NO: 84; (b) the VH comprises SEQ ID NO: 21 and the VL comprises SEQ ID NO: 23; (c) the VH comprises SEQ ID NO: 31 and the VL comprises SEQ ID NO: 33; (d) the VH comprises SEQ ID NO: 41 and the VL comprises SEQ ID NO: 43; (e) the VH comprises SEQ ID NO: 51 and the VL comprises SEQ ID NO: 53; (f) the VH comprises SEQ ID NO: 61 and the VL comprises SEQ ID NO: 63; (g) the VH comprises SEQ ID NO: 693 and the VL comprises SEQ ID NO: 66; (h) the VH comprises SEQ ID NO: 694 and the VL comprises SEQ ID NO: 69; (i) the VH comprises SEQ ID NO: 695 and the VL comprises SEQ ID NO: 72; (j) the VH comprises SEQ ID NO: 74 and the VL comprises SEQ ID NO: 76; (k) the VH comprises SEQ ID NO: 78 and the VL comprises SEQ ID NO: 80; (1) the VH comprises SEQ ID NO: 11 and the VL comprises SEQ ID NO: 13; (m) the VH comprises SEQ ID NO: 92 and the VL comprises SEQ ID NO: 94; (n) the VH comprises SEQ ID NO: 102 and the VL comprises SEQ ID NO: 103; (o) the VH comprises SEQ ID NO: 104 and the VL comprises SEQ ID NO: 105; (p) the VH comprises SEQ ID NO: 712 and the VL comprises SEQ ID NO: 713; (q) the VH comprises SEQ ID NO: 714 and the VL comprises SEQ ID NO: 715; (r) the VH comprises SEQ ID NO: 716 and the VL comprises SEQ ID NO: 717; (s) the VH comprises SEQ ID NO: 718 and the VL comprises SEQ ID NO: 719; (t) the VH comprises SEQ ID NO: 720 and the VL comprises SEQ ID NO: 721; (u) the VH comprises SEQ ID NO: 722 and the VL comprises SEQ ID NO: 723; (v) the VH comprises SEQ ID NO: 724 and the VL comprises SEQ ID NO: 725; (w) the VH comprises SEQ ID NO: 948 and the VL comprises SEQ ID NO: 949; (x) the VH comprises SEQ ID NO: 950 and the VL comprises SEQ ID NO: 951; (y) the VH comprises SEQ ID NO: 952 and the VL comprises SEQ ID NO: 953; (z) the VH comprises SEQ ID NO: 954 and the VL comprises SEQ ID NO: 955; (aa) the VH comprises SEQ ID NO: 958 and the VL comprises SEQ ID NO: 959; (bb) the VH comprises SEQ ID NO: 960 and the VL comprises SEQ ID NO: 961; (cc) the VH comprises SEQ ID NO: 964 and the VL comprises SEQ ID NO: 965; (dd) the VH comprises SEQ ID NO: 966 and the VL comprises SEQ ID NO: 967; (ee) the VH comprises SEQ ID NO: 968 and the VL comprises SEQ ID NO: 969; (ff) the VH comprises SEQ ID NO: 970 and the VL comprises SEQ ID NO: 971; (gg) the VH comprises SEQ ID NO: 972 and the VL comprises SEQ ID NO: 973; (hh) the VH comprises SEQ ID NO: 974 and the VL comprises SEQ ID NO: 975; (ii) the VH comprises SEQ ID NO: 976 and the VL comprises SEQ ID NO: 977; (jj) the VH comprises SEQ ID NO: 980 and the VL comprises SEQ ID NO: 981; (kk) the VH comprises SEQ ID NO: 982 and the VL comprises SEQ ID NO: 983; (11) the VH comprises SEQ ID NO: 984 and the VL comprises SEQ ID NO: 985; (mm) the VH comprises SEQ ID NO: 990 and the VL comprises SEQ ID NO: 991; (nn) the VH comprises SEQ ID NO: 992 and the VL comprises SEQ ID NO: 993; (oo) the VH comprises SEQ ID NO: 994 and the VL comprises SEQ ID NO: 995; (pp) the VH comprises SEQ ID NO: 996 and the VL comprises SEQ ID NO: 997; (qq) the VH comprises SEQ ID NO: 998 and the VL comprises SEQ ID NO: 999; (rr) the VH comprises SEQ ID NO: 1000 and the VL comprises SEQ ID NO: 1001; (ss) the VH comprises SEQ ID NO: 1002 and the VL comprises SEQ ID NO: 1003; (tt) the VH comprises SEQ ID NO: 1004 and the VL comprises SEQ ID NO: 1005; (uu) the VH comprises SEQ ID NO: 1006 and the VL comprises SEQ ID NO: 1007; (vv) the VH comprises SEQ ID NO: 1008 and the VL comprises SEQ ID NO: 1009; (ww) the VH comprises SEQ ID NO: 1010 and the VL comprises SEQ ID NO: 1011; (xx) the VH comprises SEQ ID NO: 1016 and the VL comprises SEQ ID NO: 1017; (yy) the VH comprises SEQ ID NO: 1018 and the VL comprises SEQ ID NO: 1019; (zz) the VH comprises SEQ ID NO: 1020 and the VL comprises SEQ ID NO: 1021; (aaa) the VH comprises SEQ ID NO: 1022 and the VL comprises SEQ ID NO: 1023; (bbb) the VH comprises SEQ ID NO: 1024 and the VL comprises SEQ ID NO: 1025; (ccc) the VH comprises SEQ ID NO: 1026 and the VL comprises SEQ ID NO: 1027; (ddd) the VH comprises SEQ ID NO: 1028 and the VL comprises SEQ ID NO: 1029; (eee) the VH comprises SEQ ID NO: 1030 and the VL comprises SEQ ID NO: 1031; (fff) the VH comprises SEQ ID NO: 1032 and the VL comprises SEQ ID NO: 1033; (ggg) the VH comprises SEQ ID NO: 1034 and the VL comprises SEQ ID NO: 1035; (hhh) the VH comprises SEQ ID NO: 1036 and the VL comprises SEQ ID NO: 1037; or (iii) the VH comprises SEQ ID NO: 1038 and the VL comprises SEQ ID NO: 1039.

Embodiment 47. The method of any one of embodiments 33-46, wherein the second antigen recognition domain comprises a single domain antibody fragment, an adnectin peptide, an affibody, an afflilin, an affimer, an affitin, an alphabody, an anticalin, an avimer, a DARPin (Designed Ankyrin Repeat Protein), a Fynomer, a Kunitz domain peptide, a monobody, a centyrin, an aptamer, a T cell receptor (TCR)-like antibody, a single chain TCR (scTCR), or a portion of any of the foregoing.

Embodiment 48. The method of any one of embodiments 33-44, wherein the second antigen recognition domain comprises a human CD27 extracellular domain.

Embodiment 49. The method of embodiment 48, wherein the human CD27 extracellular domain comprises the amino acid sequence of SEQ ID NO: 8.

Embodiment 50. The method of embodiment 48 or 49, wherein the human CD27 extracellular domain comprises a mutation.

Embodiment 51. The method of embodiment 50, wherein the mutation reduces shedding of the human CD27 extracellular domain.

Embodiment 52. The method of any one of embodiments 33-51, wherein the extracellular domain comprises a hinge.

Embodiment 53. The method of embodiment 52, wherein the hinge comprises (a) a portion of the extracellular region of CD8, CD8alpha, CD4, CD28, 4-1BB, or IgG; and/or (b) a human immunoglobulin CH2 region, a human immunoglobulin CH3 region, or both a human immunoglobulin CH2 region and a human immunoglobulin CH3 region.

Embodiment 54. The method of embodiment 52 or 53, wherein the hinge comprises a human immunoglobulin CH2 region and wherein the human immunoglobulin CH2 region is an IgG1, IgG2 or IgG4 immunoglobulin CH2 region.

Embodiment 55. The method of embodiment 52 or 53, wherein the hinge comprises a human immunoglobulin CH3 region and wherein the human immunoglobulin CH3 region is an IgG1, IgG2 or IgG4 immunoglobulin CH3 region.

Embodiment 56. The method of any one of embodiments 33-55, wherein the transmembrane domain comprises a CD8, CD16, CD27, CD28, 2B4, NKG2D, NKp44, NKp46, NKp30, NKp80, DNAM-1, CD3 zeta, CD3 epsilon, CD3 gamma, CD3 delta, CD45, CD4, CD5, CD9, CD22, CD33, CD37, CD64, CD80, CD86, CD134, CD137, CD154, ICOS/CD278, GITR/CD357, DAP10, DAP12 or erythropoietin receptor transmembrane domain, a portion of any of the foregoing, or a combination of any of the foregoing.

Embodiment 57. The method of any one of embodiments 33-56, wherein the intracellular domain comprises a costimulatory domain.

Embodiment 58. The method of any one of embodiments 33-57, wherein the intracellular domain comprises two or three costimulatory domains.

Embodiment 59. The method of embodiment 57 or 58, wherein the costimulatory domain comprises a CD28, 4-1BB, DAP10, DAP12, 2B4, OX40, OX40L, ICOS, or CD27 costimulatory domain, or a portion of any of the foregoing.

Embodiment 60. The method of any one of embodiments 33-59, wherein the intracellular domain comprises an activation domain.

Embodiment 61. The method of embodiment 60, wherein the activation domain comprises a DAP12, FCER1G, FCGR2 Å, CD3zeta activation domain, or a portion of any of the foregoing.

Embodiment 62. The method of embodiment 61, wherein the activation domain comprises the CD3zeta activation domain, or the portion thereof, and wherein the CD3zeta activation domain or the portion thereof, comprises a mutation in an ITAM domain.

Embodiment 63. The method of embodiment 62, wherein the mutation in the ITAM domain of the CD3zeta activation domain comprises point mutations of each of the two tyrosine residues in one or more of the ITAM1, ITAM2, or ITAM3 domains to a phenylalanine residue.

Embodiment 64. The method of embodiment 63, wherein the mutation in the ITAM domain of the CD3zeta activation domain comprises a deletion of one or more of the ITAM1, ITAM2, or ITAM3 domains.

Embodiment 65. The method of any one of embodiments 33-64, further comprising (e) contacting the population of NK cells with at least one (e.g., one, two, three, or more) additional polynucleotide encoding an additional exogenous polypeptide.

Embodiment 66. The method of embodiment 65, wherein a single nucleic acid molecule comprises the first polynucleotide and the at least one additional polynucleotide.

Embodiment 67. The method of embodiment 65, wherein a first nucleic acid molecule comprises the first polynucleotide and a second nucleic acid molecule comprises the at least one additional polynucleotide.

Embodiment 68. The method of any one of embodiments 65-67, wherein the additional exogenous polypeptide comprises a cytokine, chemokine, ligand, receptor, monoclonal antibody, bispecific T cell engager, peptide or enzyme, a subunit or a portion of the foregoing, or any combination of the foregoing.

Embodiment 69. The method of embodiment 68, wherein the additional exogenous polypeptide comprises a cytokine and wherein the cytokine comprises IL-15, membrane-bound IL-15 (mbIL-15), IL-2, membrane-bound IL-2, IL-12, membrane-bound IL-12, IL-18, membrane-bound IL-18, IL-21, membrane-bound IL-21, p40, LIGHT, CD40L, FLT3L, 4-1BBL, or FASL.

Embodiment 70. The method of any one of embodiments 65-67, wherein the additional exogenous polypeptide comprises IL-15RA or a fusion protein comprising IL-15 and IL-15RA.

Embodiment 71. The method of any one of embodiments 65-67, wherein the additional exogenous polypeptide is a tethered IL-21, a tethered IL-12, or a tethered IL-18.

Embodiment 72. The method of any one of embodiments 65-67, wherein the at least one additional polynucleotide encodes a first additional exogenous polypeptide and a second additional exogenous polypeptide.

Embodiment 73. The method of embodiment 72, wherein: (a) the first additional exogenous polypeptide comprises mbIL-15 and the second additional exogenous polypeptide comprises IL-15RA; or (b) the first additional exogenous polypeptide comprises soluble IL-15 and the second additional exogenous polypeptide comprises IL-15RA.

Embodiment 74. The method of embodiment 68, wherein the additional exogenous polypeptide comprises a receptor and the receptor comprises CSF-1R, a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof.

Embodiment 75. The method of embodiment 68, wherein the additional exogenous polypeptide is an enzyme and the enzyme comprises heparanase.

Embodiment 76. The method of any one of embodiments 65-67, wherein the additional exogenous polypeptide is a protein that overcomes immunosuppression of the tumor microenvironment.

Embodiment 77. The method of embodiment 76, wherein the protein comprises a TGFbeta signal converter.

Embodiment 78. The method of embodiment 77, wherein the TGFbeta signal converter comprises a TGFbeta receptor extracellular domain and an NK cell intracellular domain.

Embodiment 79. The method of embodiment 78, wherein the NK cell intracellular domain comprises DAP10 or DAP12.

Embodiment 80. The method of embodiment 76, wherein the protein comprises a TGFbeta decoy receptor.

Embodiment 81. The method of any one of embodiments 65-67, wherein the additional exogenous polypeptide comprises a second CAR, comprising an antigen recognition domain that specifically binds an antigen other than human CD70.

Embodiment 82. The method of embodiment 80, wherein the antigen other than human CD70 is CAIX, CD19, CD20, CD22, CD33, CD37, CD79a, CD79b, CD96, CD123, CD138, CLL-1, CXCR5, BCMA, FOLR2, FCRL5, FLT3, GPRC5D, HAVCR1, Her2, mesothelin, MUC16, EGFR, EGFRVIII, IL13Ra2, Trop2, GPC3, FOLR1, or GD2.

Embodiment 83. The method of any one of embodiments 65-67, wherein the additional exogenous polypeptide comprises a safety switch protein.

Embodiment 84. The method of any one of embodiments 65-83, wherein step (e) comprises use of a viral vector, electroporation, a transposon/transposase system, a lipid nanoparticle or a charge-altering releasable transporter.

Embodiment 85. The method of any one of embodiments 1-84, further comprising linking an additional exogenous polypeptide to at least one NK cell of the NK cell population by chemical conjugation or using a sortase enzyme.

Embodiment 86. A genetically engineered natural killer (NK) cell modified to have: a) a decreased level of total expressed CD70 polypeptide compared to the level of total expressed CD70 polypeptide in a wild-type NK cell, and/or b) a decreased level of surface expressed CD70 polypeptide compared to the level of surface expressed CD70 in a wild-type NK cell.

Embodiment 87. The genetically engineered NK cell of embodiment 86, wherein the genetically engineered NK cell comprises a disrupted CD70 gene.

Embodiment 88. The genetically engineered NK cell of embodiment 86 and embodiment 87, wherein the genetically engineered NK cell comprises a knockout or knockdown of a CD70 gene.

Embodiment 89. The genetically engineered NK cell of any one of embodiments 86-88, wherein the genetically engineered NK cell comprises at least about 10% less, about 20% less, about 30% less, about 40% less, about 50% less, about 60% less, about 70% less, about 80% less, or about 90% less of surface expressed CD70 polypeptide and/or total expressed CD70 polypeptide than the wild-type NK cell.

Embodiment 90. The genetically engineered NK cell of any one of embodiments 86-88, wherein the level of CD70 mRNA in the NK cell is reduced and wherein the level of CD70 mRNA is measured by Northern blot, quantitative PCR, or RNA sequencing.

Embodiment 91. The genetically engineered NK cell of any one of embodiments 86-90, wherein the level of CD70 polypeptide in the NK cell is reduced and wherein the level of CD70 polypeptide is measured by Western blot, ELISA, flow cytometry, or mass spectrometry.

Embodiment 92. The genetically engineered NK cell of any one of embodiments 86-91, wherein the genetically engineered NK cell comprises a siRNA that targets CD70 mRNA, a nucleic acid encoding a siRNA that targets CD70 mRNA, a shRNA that targets CD70 mRNA, or a nucleic acid encoding a shRNA that targets CD70 mRNA.

Embodiment 93. The genetically engineered NK cell of any one of embodiments 86-92, wherein the genetically engineered NK cell comprises a shRNA that targets CD70 mRNA or a nucleic acid encoding a shRNA that targets CD70 mRNA.

Embodiment 94. The genetically engineered NK cell of any one of embodiments 86-93, wherein the shRNA that targets CD70 mRNA comprises the nucleic acid sequence of any one of SEQ ID NOs: 2647-2652.

Embodiment 95. The genetically engineered NK cell of any one of embodiments 86-94, wherein the genetically engineered NK cell comprises an RNA guided endonuclease and a gRNA targeting a CD70 gene.

Embodiment 96. The genetically engineered NK cell of any one of embodiments 86-95, wherein the genetically engineered NK cell comprises a PEBL or a nucleic acid encoding a PEBL, wherein the PEBL comprises a first antigen recognition domain that specifically binds human CD70 and one or more of a localizing domain, an intracellular retention domain and an ER retention domain.

Embodiment 97. The genetically engineered NK cell of embodiment 96, wherein the first antigen recognition domain comprises a VH and a VL, wherein: (a) the VH comprises a CDRH1 of SEQ ID NO: 86, a CDRH2 of SEQ ID NO: 87, and a CDRH3 of SEQ ID NO: 88, and the VL comprises a CDRL1 of SEQ ID NO: 89, a CDRL2 of SEQ ID NO: 90, and a CDRL3 of SEQ ID NO: 91; (b) the VH comprises a CDRH1 of SEQ ID NO: 25, a CDRH2 of SEQ ID NO: 26, and a CDRH3 of SEQ ID NO: 27, and the VL comprises a CDRL1 of SEQ ID NO: 28, a CDRL2 of SEQ ID NO: 29, and a CDRL3 of SEQ ID NO: 30; (c) the VH comprises a CDRH1 of SEQ ID NO: 35, a CDRH2 of SEQ ID NO: 36, and a CDRH3 of SEQ ID NO: 37, and the VL comprises a CDRL1 of SEQ ID NO: 38, a CDRL2 of SEQ ID NO: 39, and a CDRL3 of SEQ ID NO: 40; (d) the VH comprises a CDRH1 of SEQ ID NO: 45, a CDRH2 of SEQ ID NO: 46, and a CDRH3 of SEQ ID NO: 47, and the VL comprises a CDRL1 of SEQ ID NO: 48, a CDRL2 of SEQ ID NO: 49, and a CDRL3 of SEQ ID NO: 50; (e) the VH comprises a CDRH1 of SEQ ID NO: 55, a CDRH2 of SEQ ID NO: 56, and a CDRH3 of SEQ ID NO: 57, and the VL comprises a CDRL1 of SEQ ID NO: 58, a CDRL2 of SEQ ID NO: 59, and a CDRL3 of SEQ ID NO: 60; (f) the VH comprises a CDRH1 of SEQ ID NO: 15, a CDRH2 of SEQ ID NO: 16, and a CDRH3 of SEQ ID NO: 17, and the VL comprises a CDRL1 of SEQ ID NO: 18, a CDRL2 of SEQ ID NO: 19, and a CDRL3 of SEQ ID NO: 20; (g) the VH comprises a CDRH1 of SEQ ID NO: 96, a CDRH2 of SEQ ID NO: 97, and a CDRH3 of SEQ ID NO: 98, and the VL comprises a CDRL1 of SEQ ID NO: 99, a CDRL2 of SEQ ID NO: 100, and a CDRL3 of SEQ ID NO: 101; (h) the VH comprises a CDRH1 of SEQ ID NO: 196, a CDRH2 of SEQ ID NO: 197, and a CDRH3 of SEQ ID NO: 198, and the VL comprises a CDRL1 of SEQ ID NO: 478, a CDRL2 of SEQ ID NO: 479, and a CDRL3 of SEQ ID NO: 480; (i) the VH comprises a CDRH1 of SEQ ID NO: 202, a CDRH2 of SEQ ID NO: 203, and a CDRH3 of SEQ ID NO: 204, and the VL comprises a CDRL1 of SEQ ID NO: 481, a CDRL2 of SEQ ID NO: 482, and a CDRL3 of SEQ ID NO: 483; (j) the VH comprises a CDRH1 of SEQ ID NO: 1170, a CDRH2 of SEQ ID NO: 1171, and a CDRH3 of SEQ ID NO: 1172, and the VL comprises a CDRL1 of SEQ ID NO: 1857, a CDRL2 of SEQ ID NO: 1858, and a CDRL3 of SEQ ID NO: 1859; (k) the VH comprises a CDRH1 of SEQ ID NO: 1173, a CDRH2 of SEQ ID NO: 1174, and a CDRH3 of SEQ ID NO: 1175, and the VL comprises a CDRL1 of SEQ ID NO: 1860, a CDRL2 of SEQ ID NO: 1861, and a CDRL3 of SEQ ID NO: 1862; (1) the VH comprises a CDRH1 of SEQ ID NO: 1176, a CDRH2 of SEQ ID NO: 1177, and a CDRH3 of SEQ ID NO: 1178, and the VL comprises a CDRL1 of SEQ ID NO: 1863, a CDRL2 of SEQ ID NO: 1864, and a CDRL3 of SEQ ID NO: 1865; (m) the VH comprises a CDRH1 of SEQ ID NO: 1179, a CDRH2 of SEQ ID NO: 1180, and a CDRH3 of SEQ ID NO: 1181, and the VL comprises a CDRL1 of SEQ ID NO: 1866, a CDRL2 of SEQ ID NO: 1867, and a CDRL3 of SEQ ID NO: 1868; (n) the VH comprises a CDRH1 of SEQ ID NO: 1182, a CDRH2 of SEQ ID NO: 1183, and a CDRH3 of SEQ ID NO: 1184, and the VL comprises a CDRL1 of SEQ ID NO: 1869, a CDRL2 of SEQ ID NO: 1870, and a CDRL3 of SEQ ID NO: 1871; (o) the VH comprises a CDRH1 of SEQ ID NO: 1185, a CDRH2 of SEQ ID NO: 1186, and a CDRH3 of SEQ ID NO: 1187, and the VL comprises a CDRL1 of SEQ ID NO: 1872, a CDRL2 of SEQ ID NO: 1873, and a CDRL3 of SEQ ID NO: 1874; (p) the VH comprises a CDRH1 of SEQ ID NO: 1188, a CDRH2 of SEQ ID NO: 1189, and a CDRH3 of SEQ ID NO: 1190, and the VL comprises a CDRL1 of SEQ ID NO: 1875, a CDRL2 of SEQ ID NO: 1876, and a CDRL3 of SEQ ID NO: 1877; (q) the VH comprises a CDRH1 of SEQ ID NO: 1524, a CDRH2 of SEQ ID NO: 1525, and a CDRH3 of SEQ ID NO: 1526, and the VL comprises a CDRL1 of SEQ ID NO: 2211, a CDRL2 of SEQ ID NO: 2212, and a CDRL3 of SEQ ID NO: 2213; (r) the VH comprises a CDRH1 of SEQ ID NO: 1527, a CDRH2 of SEQ ID NO: 1528, and a CDRH3 of SEQ ID NO: 1529, and the VL comprises a CDRL1 of SEQ ID NO: 2214, a CDRL2 of SEQ ID NO: 2215, and a CDRL3 of SEQ ID NO: 2216; (s) the VH comprises a CDRH1 of SEQ ID NO: 1530, a CDRH2 of SEQ ID NO: 1531, and a CDRH3 of SEQ ID NO: 1532, and the VL comprises a CDRL1 of SEQ ID NO: 2217, a CDRL2 of SEQ ID NO: 2218, and a CDRL3 of SEQ ID NO: 2219; (t) the VH comprises a CDRH1 of SEQ ID NO: 1533, a CDRH2 of SEQ ID NO: 1534, and a CDRH3 of SEQ ID NO: 1535, and the VL comprises a CDRL1 of SEQ ID NO: 2220, a CDRL2 of SEQ ID NO: 2221, and a CDRL3 of SEQ ID NO: 2222; (u) the VH comprises a CDRH1 of SEQ ID NO: 1539, a CDRH2 of SEQ ID NO: 1540, and a CDRH3 of SEQ ID NO: 1541, and the VL comprises a CDRL1 of SEQ ID NO: 2226, a CDRL2 of SEQ ID NO: 2227, and a CDRL3 of SEQ ID NO: 2228; (v) the VH comprises a CDRH1 of SEQ ID NO: 1542, a CDRH2 of SEQ ID NO: 1543, and a CDRH3 of SEQ ID NO: 1544, and the VL comprises a CDRL1 of SEQ ID NO: 2229, a CDRL2 of SEQ ID NO: 2230, and a CDRL3 of SEQ ID NO: 2231; (w) the VH comprises a CDRH1 of SEQ ID NO: 1548, a CDRH2 of SEQ ID NO: 1549, and a CDRH3 of SEQ ID NO: 1550, and the VL comprises a CDRL1 of SEQ ID NO: 2235, a CDRL2 of SEQ ID NO: 2236, and a CDRL3 of SEQ ID NO: 2237; (x) the VH comprises a CDRH1 of SEQ ID NO: 1551, a CDRH2 of SEQ ID NO: 1552, and a CDRH3 of SEQ ID NO: 1553, and the VL comprises a CDRL1 of SEQ ID NO: 2238, a CDRL2 of SEQ ID NO: 2239, and a CDRL3 of SEQ ID NO: 2240; (y) the VH comprises a CDRH1 of SEQ ID NO: 1554, a CDRH2 of SEQ ID NO: 1555, and a CDRH3 of SEQ ID NO: 1556, and the VL comprises a CDRL1 of SEQ ID NO: 2241, a CDRL2 of SEQ ID NO: 2242, and a CDRL3 of SEQ ID NO: 2243; (z) the VH comprises a CDRH1 of SEQ ID NO: 1557, a CDRH2 of SEQ ID NO: 1558, and a CDRH3 of SEQ ID NO: 1559, and the VL comprises a CDRL1 of SEQ ID NO: 2244, a CDRL2 of SEQ ID NO: 2245, and a CDRL3 of SEQ ID NO: 2246; (aa) the VH comprises a CDRH1 of SEQ ID NO: 1560, a CDRH2 of SEQ ID NO: 1561, and a CDRH3 of SEQ ID NO: 1562, and the VL comprises a CDRL1 of SEQ ID NO: 2247, a CDRL2 of SEQ ID NO: 2248, and a CDRL3 of SEQ ID NO: 2249; (bb) the VH comprises a CDRH1 of SEQ ID NO: 1563, a CDRH2 of SEQ ID NO: 1564, and a CDRH3 of SEQ ID NO: 1565, and the VL comprises a CDRL1 of SEQ ID NO: 2250, a CDRL2 of SEQ ID NO: 2251, and a CDRL3 of SEQ ID NO: 2252; (cc) the VH comprises a CDRH1 of SEQ ID NO: 1566, a CDRH2 of SEQ ID NO: 1567, and a CDRH3 of SEQ ID NO: 1568, and the VL comprises a CDRL1 of SEQ ID NO: 2253, a CDRL2 of SEQ ID NO: 2254, and a CDRL3 of SEQ ID NO: 2255; (dd) the VH comprises a CDRH1 of SEQ ID NO: 1572, a CDRH2 of SEQ ID NO: 1573, and a CDRH3 of SEQ ID NO: 1574, and the VL comprises a CDRL1 of SEQ ID NO: 2259, a CDRL2 of SEQ ID NO: 2260, and a CDRL3 of SEQ ID NO: 2261; (ee) the VH comprises a CDRH1 of SEQ ID NO: 1575, a CDRH2 of SEQ ID NO: 1576, and a CDRH3 of SEQ ID NO: 1577, and the VL comprises a CDRL1 of SEQ ID NO: 2262, a CDRL2 of SEQ ID NO: 2263, and a CDRL3 of SEQ ID NO: 2264; (ff) the VH comprises a CDRH1 of SEQ ID NO: 1578, a CDRH2 of SEQ ID NO: 1579, and a CDRH3 of SEQ ID NO: 1580, and the VL comprises a CDRL1 of SEQ ID NO: 2265, a CDRL2 of SEQ ID NO: 2266, and a CDRL3 of SEQ ID NO: 2267; (gg) the VH comprises a CDRH1 of SEQ ID NO: 1587, a CDRH2 of SEQ ID NO: 1588, and a CDRH3 of SEQ ID NO: 1589, and the VL comprises a CDRL1 of SEQ ID NO: 2274, a CDRL2 of SEQ ID NO: 2275, and a CDRL3 of SEQ ID NO: 2276; (hh) the VH comprises a CDRH1 of SEQ ID NO: 1590, a CDRH2 of SEQ ID NO: 1591, and a CDRH3 of SEQ ID NO: 1592, and the VL comprises a CDRL1 of SEQ ID NO: 2277, a CDRL2 of SEQ ID NO: 2278, and a CDRL3 of SEQ ID NO: 2279; (ii) the VH comprises a CDRH1 of SEQ ID NO: 1593, a CDRH2 of SEQ ID NO: 1594, and a CDRH3 of SEQ ID NO: 1595, and the VL comprises a CDRL1 of SEQ ID NO: 2280, a CDRL2 of SEQ ID NO: 2281, and a CDRL3 of SEQ ID NO: 2282; (jj) the VH comprises a CDRH1 of SEQ ID NO: 1596, a CDRH2 of SEQ ID NO: 1597, and a CDRH3 of SEQ ID NO: 1598, and the VL comprises a CDRL1 of SEQ ID NO: 2283, a CDRL2 of SEQ ID NO: 2284, and a CDRL3 of SEQ ID NO: 2285; (kk) the VH comprises a CDRH1 of SEQ ID NO: 1599, a CDRH2 of SEQ ID NO: 1560, and a CDRH3 of SEQ ID NO: 1561, and the VL comprises a CDRL1 of SEQ ID NO: 2286, a CDRL2 of SEQ ID NO: 2287, and a CDRL3 of SEQ ID NO: 2288; (11) the VH comprises a CDRH1 of SEQ ID NO: 1602, a CDRH2 of SEQ ID NO: 1603, and a CDRH3 of SEQ ID NO: 1604, and the VL comprises a CDRL1 of SEQ ID NO: 2289, a CDRL2 of SEQ ID NO: 2290, and a CDRL3 of SEQ ID NO: 2291; (mm) the VH comprises a CDRH1 of SEQ ID NO: 1605, a CDRH2 of SEQ ID NO: 1606, and a CDRH3 of SEQ ID NO: 1607, and the VL comprises a CDRL1 of SEQ ID NO: 2292, a CDRL2 of SEQ ID NO: 2293, and a CDRL3 of SEQ ID NO: 2294; (nn) the VH comprises a CDRH1 of SEQ ID NO: 1608, a CDRH2 of SEQ ID NO: 1609, and a CDRH3 of SEQ ID NO: 1610, and the VL comprises a CDRL1 of SEQ ID NO: 2295, a CDRL2 of SEQ ID NO: 2296, and a CDRL3 of SEQ ID NO: 2297; (oo) the VH comprises a CDRH1 of SEQ ID NO: 1611, a CDRH2 of SEQ ID NO: 1612, and a CDRH3 of SEQ ID NO: 1613, and the VL comprises a CDRL1 of SEQ ID NO: 2298, a CDRL2 of SEQ ID NO: 2299, and a CDRL3 of SEQ ID NO: 2300; (pp) the VH comprises a CDRH1 of SEQ ID NO: 1614, a CDRH2 of SEQ ID NO: 1615, and a CDRH3 of SEQ ID NO: 1616, and the VL comprises a CDRL1 of SEQ ID NO: 2301, a CDRL2 of SEQ ID NO: 2302, and a CDRL3 of SEQ ID NO: 2303; (qq) the VH comprises a CDRH1 of SEQ ID NO: 1617, a CDRH2 of SEQ ID NO: 1618, and a CDRH3 of SEQ ID NO: 1619, and the VL comprises a CDRL1 of SEQ ID NO: 2304, a CDRL2 of SEQ ID NO: 2305, and a CDRL3 of SEQ ID NO: 2306; (rr) the VH comprises a CDRH1 of SEQ ID NO: 1626, a CDRH2 of SEQ ID NO: 1627, and a CDRH3 of SEQ ID NO: 1628, and the VL comprises a CDRL1 of SEQ ID NO: 2313, a CDRL2 of SEQ ID NO: 2314, and a CDRL3 of SEQ ID NO: 2315; (ss) the VH comprises a CDRH1 of SEQ ID NO: 1629, a CDRH2 of SEQ ID NO: 1630, and a CDRH3 of SEQ ID NO: 1631, and the VL comprises a CDRL1 of SEQ ID NO: 2316, a CDRL2 of SEQ ID NO: 2317, and a CDRL3 of SEQ ID NO: 2318; (tt) the VH comprises a CDRH1 of SEQ ID NO: 1632, a CDRH2 of SEQ ID NO: 1633, and a CDRH3 of SEQ ID NO: 1634, and the VL comprises a CDRL1 of SEQ ID NO: 2319, a CDRL2 of SEQ ID NO: 2320, and a CDRL3 of SEQ ID NO: 2321; (uu) the VH comprises a CDRH1 of SEQ ID NO: 1635, a CDRH2 of SEQ ID NO: 1636, and a CDRH3 of SEQ ID NO: 1637, and the VL comprises a CDRL1 of SEQ ID NO: 2322, a CDRL2 of SEQ ID NO: 2323, and a CDRL3 of SEQ ID NO: 2324; (vv) the VH comprises a CDRH1 of SEQ ID NO: 1638, a CDRH2 of SEQ ID NO: 1639, and a CDRH3 of SEQ ID NO: 1640, and the VL comprises a CDRL1 of SEQ ID NO: 2325, a CDRL2 of SEQ ID NO: 2326, and a CDRL3 of SEQ ID NO: 2327; (ww) the VH comprises a CDRH1 of SEQ ID NO: 1641, a CDRH2 of SEQ ID NO: 1642, and a CDRH3 of SEQ ID NO: 1643, and the VL comprises a CDRL1 of SEQ ID NO: 2328, a CDRL2 of SEQ ID NO: 2329, and a CDRL3 of SEQ ID NO: 2330; (xx) the VH comprises a CDRH1 of SEQ ID NO: 1644, a CDRH2 of SEQ ID NO: 1645, and a CDRH3 of SEQ ID NO: 1646, and the VL comprises a CDRL1 of SEQ ID NO: 2331, a CDRL2 of SEQ ID NO: 2332, and a CDRL3 of SEQ ID NO: 2333; (yy) the VH comprises a CDRH1 of SEQ ID NO: 1647, a CDRH2 of SEQ ID NO: 1648, and a CDRH3 of SEQ ID NO: 1649, and the VL comprises a CDRL1 of SEQ ID NO: 2334, a CDRL2 of SEQ ID NO: 2335, and a CDRL3 of SEQ ID NO: 2336; (zz) the VH comprises a CDRH1 of SEQ ID NO: 1650, a CDRH2 of SEQ ID NO: 1651, and a CDRH3 of SEQ ID NO: 1652, and the VL comprises a CDRL1 of SEQ ID NO: 2337, a CDRL2 of SEQ ID NO: 2338, and a CDRL3 of SEQ ID NO: 2339; (aaa) the VH comprises a CDRH1 of SEQ ID NO: 1653, a CDRH2 of SEQ ID NO: 1654, and a CDRH3 of SEQ ID NO: 1655, and the VL comprises a CDRL1 of SEQ ID NO: 2340, a CDRL2 of SEQ ID NO: 2341, and a CDRL3 of SEQ ID NO: 2342; (bbb) the VH comprises a CDRH1 of SEQ ID NO: 1656, a CDRH2 of SEQ ID NO: 1657, and a CDRH3 of SEQ ID NO: 1658, and the VL comprises a CDRL1 of SEQ ID NO: 2343, a CDRL2 of SEQ ID NO: 2344, and a CDRL3 of SEQ ID NO: 2345; or (ccc) the VH comprises a CDRH1 of SEQ ID NO: 1659, a CDRH2 of SEQ ID NO: 1660, and a CDRH3 of SEQ ID NO: 1661, and the VL comprises a CDRL1 of SEQ ID NO: 2346, a CDRL2 of SEQ ID NO: 2347, and a CDRL3 of SEQ ID NO: 2348.

Embodiment 98. The genetically engineered NK cell of embodiment 96 or 97, wherein the first antigen recognition domain comprises a VH and a VL, wherein:

(a) the VH comprises SEQ ID NO: 82 and the VL comprises SEQ ID NO: 84; (b) the VH comprises SEQ ID NO: 21 and the VL comprises SEQ ID NO: 23; (c) the VH comprises SEQ ID NO: 31 and the VL comprises SEQ ID NO: 33; (d) the VH comprises SEQ ID NO: 41 and the VL comprises SEQ ID NO: 43; (e) the VH comprises SEQ ID NO: 51 and the VL comprises SEQ ID NO: 53; (f) the VH comprises SEQ ID NO: 61 and the VL comprises SEQ ID NO: 63; (g) the VH comprises SEQ ID NO: 693 and the VL comprises SEQ ID NO: 66; (h) the VH comprises SEQ ID NO: 694 and the VL comprises SEQ ID NO: 69; (i) the VH comprises SEQ ID NO: 695 and the VL comprises SEQ ID NO: 72; (j) the VH comprises SEQ ID NO: 74 and the VL comprises SEQ ID NO: 76; (k) the VH comprises SEQ ID NO: 78 and the VL comprises SEQ ID NO: 80; (1) the VH comprises SEQ ID NO: 11 and the VL comprises SEQ ID NO: 13; (m) the VH comprises SEQ ID NO: 92 and the VL comprises SEQ ID NO: 94; (n) the VH comprises SEQ ID NO: 102 and the VL comprises SEQ ID NO: 103; (o) the VH comprises SEQ ID NO: 104 and the VL comprises SEQ ID NO: 105; (p) the VH comprises SEQ ID NO: 712 and the VL comprises SEQ ID NO: 713; (q) the VH comprises SEQ ID NO: 714 and the VL comprises SEQ ID NO: 715; (r) the VH comprises SEQ ID NO: 716 and the VL comprises SEQ ID NO: 717; (s) the VH comprises SEQ ID NO: 718 and the VL comprises SEQ ID NO: 719; (t) the VH comprises SEQ ID NO: 720 and the VL comprises SEQ ID NO: 721; (u) the VH comprises SEQ ID NO: 722 and the VL comprises SEQ ID NO: 723; (v) the VH comprises SEQ ID NO: 724 and the VL comprises SEQ ID NO: 725; (w) the VH comprises SEQ ID NO: 948 and the VL comprises SEQ ID NO: 949; (x) the VH comprises SEQ ID NO: 950 and the VL comprises SEQ ID NO: 951; (y) the VH comprises SEQ ID NO: 952 and the VL comprises SEQ ID NO: 953; (z) the VH comprises SEQ ID NO: 954 and the VL comprises SEQ ID NO: 955; (aa) the VH comprises SEQ ID NO: 958 and the VL comprises SEQ ID NO: 959; (bb) the VH comprises SEQ ID NO: 960 and the VL comprises SEQ ID NO: 961; (cc) the VH comprises SEQ ID NO: 964 and the VL comprises SEQ ID NO: 965; (dd) the VH comprises SEQ ID NO: 966 and the VL comprises SEQ ID NO: 967; (ee) the VH comprises SEQ ID NO: 968 and the VL comprises SEQ ID NO: 969; (ff) the VH comprises SEQ ID NO: 970 and the VL comprises SEQ ID NO: 971; (gg) the VH comprises SEQ ID NO: 972 and the VL comprises SEQ ID NO: 973; (hh) the VH comprises SEQ ID NO: 974 and the VL comprises SEQ ID NO: 975; (ii) the VH comprises SEQ ID NO: 976 and the VL comprises SEQ ID NO: 977; (jj) the VH comprises SEQ ID NO: 980 and the VL comprises SEQ ID NO: 981; (kk) the VH comprises SEQ ID NO: 982 and the VL comprises SEQ ID NO: 983; (11) the VH comprises SEQ ID NO: 984 and the VL comprises SEQ ID NO: 985; (mm) the VH comprises SEQ ID NO: 990 and the VL comprises SEQ ID NO: 991; (nn) the VH comprises SEQ ID NO: 992 and the VL comprises SEQ ID NO: 993; (oo) the VH comprises SEQ ID NO: 994 and the VL comprises SEQ ID NO: 995; (pp) the VH comprises SEQ ID NO: 996 and the VL comprises SEQ ID NO: 997; (qq) the VH comprises SEQ ID NO: 998 and the VL comprises SEQ ID NO: 999; (rr) the VH comprises SEQ ID NO: 1000 and the VL comprises SEQ ID NO: 1001; (ss) the VH comprises SEQ ID NO: 1002 and the VL comprises SEQ ID NO: 1003; (tt) the VH comprises SEQ ID NO: 1004 and the VL comprises SEQ ID NO: 1005; (uu) the VH comprises SEQ ID NO: 1006 and the VL comprises SEQ ID NO: 1007; (vv) the VH comprises SEQ ID NO: 1008 and the VL comprises SEQ ID NO: 1009; (ww) the VH comprises SEQ ID NO: 1010 and the VL comprises SEQ ID NO: 1011; (xx) the VH comprises SEQ ID NO: 1016 and the VL comprises SEQ ID NO: 1017; (yy) the VH comprises SEQ ID NO: 1018 and the VL comprises SEQ ID NO: 1019; (zz) the VH comprises SEQ ID NO: 1020 and the VL comprises SEQ ID NO: 1021; (aaa) the VH comprises SEQ ID NO: 1022 and the VL comprises SEQ ID NO: 1023; (bbb) the VH comprises SEQ ID NO: 1024 and the VL comprises SEQ ID NO: 1025; (ccc) the VH comprises SEQ ID NO: 1026 and the VL comprises SEQ ID NO: 1027; (ddd) the VH comprises SEQ ID NO: 1028 and the VL comprises SEQ ID NO: 1029; (eee) the VH comprises SEQ ID NO: 1030 and the VL comprises SEQ ID NO: 1031; (fff) the VH comprises SEQ ID NO: 1032 and the VL comprises SEQ ID NO: 1033; (ggg) the VH comprises SEQ ID NO: 1034 and the VL comprises SEQ ID NO: 1035; (hhh) the VH comprises SEQ ID NO: 1036 and the VL comprises SEQ ID NO: 1037; or (iii) the VH comprises SEQ ID NO: 1038 and the VL comprises SEQ ID NO: 1039.

Embodiment 99. The genetically engineered NK cell of any one of embodiments 86-98, wherein the genetically engineered NK cell is derived from umbilical cord blood cells, PBMCs, mobilized PBSCs, unmobilized PBSCs, hESCs, iPSCs, MSCs, HSCs, bone marrow or CD34⁺ cells.

Embodiment 100. The genetically engineered NK cell of any one of embodiments 86-99, wherein the genetically engineered NK cell is a human NK cell.

Embodiment 101. The genetically engineered NK cell of any of embodiment 86-100, wherein the genetically engineered NK cell comprises a knockout or knockdown of a cellular gene.

Embodiment 102. The genetically engineered NK cell of embodiment 101, wherein the cellular gene is selected from CD70, CISH, PD1, TGFBR1, TFGBR2, or a combination thereof.

Embodiment 103. The genetically engineered NK cell of any one of embodiments 86-102, wherein the genetically engineered NK cell comprises a first CAR and/or a polynucleotide encoding the first CAR, wherein the first CAR comprises (a) an extracellular domain comprising a second antigen recognition domain that specifically binds human CD70; (b) a transmembrane domain; and (c) an intracellular domain.

Embodiment 104. The genetically engineered NK cell of embodiment 103, wherein the genetically engineered NK cell expresses the CAR.

Embodiment 105. The genetically engineered NK cell of embodiment 103 or 104, wherein the second antigen recognition domain comprises a scFv comprising a VH and a VL, wherein: (a) the VH comprises a CDRH1 of SEQ ID NO: 86, a CDRH2 of SEQ ID NO: 87, and a CDRH3 of SEQ ID NO: 88, and the VL comprises a CDRL1 of SEQ ID NO: 89, a CDRL2 of SEQ ID NO: 90, and a CDRL3 of SEQ ID NO: 91; (b) the VH comprises a CDRH1 of SEQ ID NO: 25, a CDRH2 of SEQ ID NO: 26, and a CDRH3 of SEQ ID NO: 27, and the VL comprises a CDRL1 of SEQ ID NO: 28, a CDRL2 of SEQ ID NO: 29, and a CDRL3 of SEQ ID NO: 30;

(c) the VH comprises a CDRH1 of SEQ ID NO: 35, a CDRH2 of SEQ ID NO: 36, and a CDRH3 of SEQ ID NO: 37, and the VL comprises a CDRL1 of SEQ ID NO: 38, a CDRL2 of SEQ ID NO: 39, and a CDRL3 of SEQ ID NO: 40; (d) the VH comprises a CDRH1 of SEQ ID NO: 45, a CDRH2 of SEQ ID NO: 46, and a CDRH3 of SEQ ID NO: 47, and the VL comprises a CDRL1 of SEQ ID NO: 48, a CDRL2 of SEQ ID NO: 49, and a CDRL3 of SEQ ID NO: 50; (e) the VH comprises a CDRH1 of SEQ ID NO: 55, a CDRH2 of SEQ ID NO: 56, and a CDRH3 of SEQ ID NO: 57, and the VL comprises a CDRL1 of SEQ ID NO: 58, a CDRL2 of SEQ ID NO: 59, and a CDRL3 of SEQ ID NO: 60; (f) the VH comprises a CDRH1 of SEQ ID NO: 15, a CDRH2 of SEQ ID NO: 16, and a CDRH3 of SEQ ID NO: 17, and the VL comprises a CDRL1 of SEQ ID NO: 18, a CDRL2 of SEQ ID NO: 19, and a CDRL3 of SEQ ID NO: 20; (g) the VH comprises a CDRH1 of SEQ ID NO: 96, a CDRH2 of SEQ ID NO: 97, and a CDRH3 of SEQ ID NO: 98, and the VL comprises a CDRL1 of SEQ ID NO: 99, a CDRL2 of SEQ ID NO: 100, and a CDRL3 of SEQ ID NO: 101; (h) the VH comprises a CDRH1 of SEQ ID NO: 196, a CDRH2 of SEQ ID NO: 197, and a CDRH3 of SEQ ID NO: 198, and the VL comprises a CDRL1 of SEQ ID NO: 478, a CDRL2 of SEQ ID NO: 479, and a CDRL3 of SEQ ID NO: 480; (i) the VH comprises a CDRH1 of SEQ ID NO: 202, a CDRH2 of SEQ ID NO: 203, and a CDRH3 of SEQ ID NO: 204, and the VL comprises a CDRL1 of SEQ ID NO: 481, a CDRL2 of SEQ ID NO: 482, and a CDRL3 of SEQ ID NO: 483; (j) the VH comprises a CDRH1 of SEQ ID NO: 1170, a CDRH2 of SEQ ID NO: 1171, and a CDRH3 of SEQ ID NO: 1172, and the VL comprises a CDRL1 of SEQ ID NO: 1857, a CDRL2 of SEQ ID NO: 1858, and a CDRL3 of SEQ ID NO: 1859; (k) the VH comprises a CDRH1 of SEQ ID NO: 1173, a CDRH2 of SEQ ID NO: 1174, and a CDRH3 of SEQ ID NO: 1175, and the VL comprises a CDRL1 of SEQ ID NO: 1860, a CDRL2 of SEQ ID NO: 1861, and a CDRL3 of SEQ ID NO: 1862; (1) the VH comprises a CDRH1 of SEQ ID NO: 1176, a CDRH2 of SEQ ID NO: 1177, and a CDRH3 of SEQ ID NO: 1178, and the VL comprises a CDRL1 of SEQ ID NO: 1863, a CDRL2 of SEQ ID NO: 1864, and a CDRL3 of SEQ ID NO: 1865; (m) the VH comprises a CDRH1 of SEQ ID NO: 1179, a CDRH2 of SEQ ID NO: 1180, and a CDRH3 of SEQ ID NO: 1181, and the VL comprises a CDRL1 of SEQ ID NO: 1866, a CDRL2 of SEQ ID NO: 1867, and a CDRL3 of SEQ ID NO: 1868; (n) the VH comprises a CDRH1 of SEQ ID NO: 1182, a CDRH2 of SEQ ID NO: 1183, and a CDRH3 of SEQ ID NO: 1184, and the VL comprises a CDRL1 of SEQ ID NO: 1869, a CDRL2 of SEQ ID NO: 1870, and a CDRL3 of SEQ ID NO: 1871; (o) the VH comprises a CDRH1 of SEQ ID NO: 1185, a CDRH2 of SEQ ID NO: 1186, and a CDRH3 of SEQ ID NO: 1187, and the VL comprises a CDRL1 of SEQ ID NO: 1872, a CDRL2 of SEQ ID NO: 1873, and a CDRL3 of SEQ ID NO: 1874; (p) the VH comprises a CDRH1 of SEQ ID NO: 1188, a CDRH2 of SEQ ID NO: 1189, and a CDRH3 of SEQ ID NO: 1190, and the VL comprises a CDRL1 of SEQ ID NO: 1875, a CDRL2 of SEQ ID NO: 1876, and a CDRL3 of SEQ ID NO: 1877; (q) the VH comprises a CDRH1 of SEQ ID NO: 1524, a CDRH2 of SEQ ID NO: 1525, and a CDRH3 of SEQ ID NO: 1526, and the VL comprises a CDRL1 of SEQ ID NO: 2211, a CDRL2 of SEQ ID NO: 2212, and a CDRL3 of SEQ ID NO: 2213; (r) the VH comprises a CDRH1 of SEQ ID NO: 1527, a CDRH2 of SEQ ID NO: 1528, and a CDRH3 of SEQ ID NO: 1529, and the VL comprises a CDRL1 of SEQ ID NO: 2214, a CDRL2 of SEQ ID NO: 2215, and a CDRL3 of SEQ ID NO: 2216; (s) the VH comprises a CDRH1 of SEQ ID NO: 1530, a CDRH2 of SEQ ID NO: 1531, and a CDRH3 of SEQ ID NO: 1532, and the VL comprises a CDRL1 of SEQ ID NO: 2217, a CDRL2 of SEQ ID NO: 2218, and a CDRL3 of SEQ ID NO: 2219; (t) the VH comprises a CDRH1 of SEQ ID NO: 1533, a CDRH2 of SEQ ID NO: 1534, and a CDRH3 of SEQ ID NO: 1535, and the VL comprises a CDRL1 of SEQ ID NO: 2220, a CDRL2 of SEQ ID NO: 2221, and a CDRL3 of SEQ ID NO: 2222; (u) the VH comprises a CDRH1 of SEQ ID NO: 1539, a CDRH2 of SEQ ID NO: 1540, and a CDRH3 of SEQ ID NO: 1541, and the VL comprises a CDRL1 of SEQ ID NO: 2226, a CDRL2 of SEQ ID NO: 2227, and a CDRL3 of SEQ ID NO: 2228; (v) the VH comprises a CDRH1 of SEQ ID NO: 1542, a CDRH2 of SEQ ID NO: 1543, and a CDRH3 of SEQ ID NO: 1544, and the VL comprises a CDRL1 of SEQ ID NO: 2229, a CDRL2 of SEQ ID NO: 2230, and a CDRL3 of SEQ ID NO: 2231; (w) the VH comprises a CDRH1 of SEQ ID NO: 1548, a CDRH2 of SEQ ID NO: 1549, and a CDRH3 of SEQ ID NO: 1550, and the VL comprises a CDRL1 of SEQ ID NO: 2235, a CDRL2 of SEQ ID NO: 2236, and a CDRL3 of SEQ ID NO: 2237; (x) the VH comprises a CDRH1 of SEQ ID NO: 1551, a CDRH2 of SEQ ID NO: 1552, and a CDRH3 of SEQ ID NO: 1553, and the VL comprises a CDRL1 of SEQ ID NO: 2238, a CDRL2 of SEQ ID NO: 2239, and a CDRL3 of SEQ ID NO: 2240; (y) the VH comprises a CDRH1 of SEQ ID NO: 1554, a CDRH2 of SEQ ID NO: 1555, and a CDRH3 of SEQ ID NO: 1556, and the VL comprises a CDRL1 of SEQ ID NO: 2241, a CDRL2 of SEQ ID NO: 2242, and a CDRL3 of SEQ ID NO: 2243; (z) the VH comprises a CDRH1 of SEQ ID NO: 1557, a CDRH2 of SEQ ID NO: 1558, and a CDRH3 of SEQ ID NO: 1559, and the VL comprises a CDRL1 of SEQ ID NO: 2244, a CDRL2 of SEQ ID NO: 2245, and a CDRL3 of SEQ ID NO: 2246; (aa) the VH comprises a CDRH1 of SEQ ID NO: 1560, a CDRH2 of SEQ ID NO: 1561, and a CDRH3 of SEQ ID NO: 1562, and the VL comprises a CDRL1 of SEQ ID NO: 2247, a CDRL2 of SEQ ID NO: 2248, and a CDRL3 of SEQ ID NO: 2249; (bb) the VH comprises a CDRH1 of SEQ ID NO: 1563, a CDRH2 of SEQ ID NO: 1564, and a CDRH3 of SEQ ID NO: 1565, and the VL comprises a CDRL1 of SEQ ID NO: 2250, a CDRL2 of SEQ ID NO: 2251, and a CDRL3 of SEQ ID NO: 2252; (cc) the VH comprises a CDRH1 of SEQ ID NO: 1566, a CDRH2 of SEQ ID NO: 1567, and a CDRH3 of SEQ ID NO: 1568, and the VL comprises a CDRL1 of SEQ ID NO: 2253, a CDRL2 of SEQ ID NO: 2254, and a CDRL3 of SEQ ID NO: 2255; (dd) the VH comprises a CDRH1 of SEQ ID NO: 1572, a CDRH2 of SEQ ID NO: 1573, and a CDRH3 of SEQ ID NO: 1574, and the VL comprises a CDRL1 of SEQ ID NO: 2259, a CDRL2 of SEQ ID NO: 2260, and a CDRL3 of SEQ ID NO: 2261; (ee) the VH comprises a CDRH1 of SEQ ID NO: 1575, a CDRH2 of SEQ ID NO: 1576, and a CDRH3 of SEQ ID NO: 1577, and the VL comprises a CDRL1 of SEQ ID NO: 2262, a CDRL2 of SEQ ID NO: 2263, and a CDRL3 of SEQ ID NO: 2264; (ff) the VH comprises a CDRH1 of SEQ ID NO: 1578, a CDRH2 of SEQ ID NO: 1579, and a CDRH3 of SEQ ID NO: 1580, and the VL comprises a CDRL1 of SEQ ID NO: 2265, a CDRL2 of SEQ ID NO: 2266, and a CDRL3 of SEQ ID NO: 2267; (gg) the VH comprises a CDRH1 of SEQ ID NO: 1587, a CDRH2 of SEQ ID NO: 1588, and a CDRH3 of SEQ ID NO: 1589, and the VL comprises a CDRL1 of SEQ ID NO: 2274, a CDRL2 of SEQ ID NO: 2275, and a CDRL3 of SEQ ID NO: 2276; (hh) the VH comprises a CDRH1 of SEQ ID NO: 1590, a CDRH2 of SEQ ID NO: 1591, and a CDRH3 of SEQ ID NO: 1592, and the VL comprises a CDRL1 of SEQ ID NO: 2277, a CDRL2 of SEQ ID NO: 2278, and a CDRL3 of SEQ ID NO: 2279; (ii) the VH comprises a CDRH1 of SEQ ID NO: 1593, a CDRH2 of SEQ ID NO: 1594, and a CDRH3 of SEQ ID NO: 1595, and the VL comprises a CDRL1 of SEQ ID NO: 2280, a CDRL2 of SEQ ID NO: 2281, and a CDRL3 of SEQ ID NO: 2282; (jj) the VH comprises a CDRH1 of SEQ ID NO: 1596, a CDRH2 of SEQ ID NO: 1597, and a CDRH3 of SEQ ID NO: 1598, and the VL comprises a CDRL1 of SEQ ID NO: 2283, a CDRL2 of SEQ ID NO: 2284, and a CDRL3 of SEQ ID NO: 2285; (kk) the VH comprises a CDRH1 of SEQ ID NO: 1599, a CDRH2 of SEQ ID NO: 1560, and a CDRH3 of SEQ ID NO: 1561, and the VL comprises a CDRL1 of SEQ ID NO: 2286, a CDRL2 of SEQ ID NO: 2287, and a CDRL3 of SEQ ID NO: 2288; (11) the VH comprises a CDRH1 of SEQ ID NO: 1602, a CDRH2 of SEQ ID NO: 1603, and a CDRH3 of SEQ ID NO: 1604, and the VL comprises a CDRL1 of SEQ ID NO: 2289, a CDRL2 of SEQ ID NO: 2290, and a CDRL3 of SEQ ID NO: 2291; (mm) the VH comprises a CDRH1 of SEQ ID NO: 1605, a CDRH2 of SEQ ID NO: 1606, and a CDRH3 of SEQ ID NO: 1607, and the VL comprises a CDRL1 of SEQ ID NO: 2292, a CDRL2 of SEQ ID NO: 2293, and a CDRL3 of SEQ ID NO: 2294; (nn) the VH comprises a CDRH1 of SEQ ID NO: 1608, a CDRH2 of SEQ ID NO: 1609, and a CDRH3 of SEQ ID NO: 1610, and the VL comprises a CDRL1 of SEQ ID NO: 2295, a CDRL2 of SEQ ID NO: 2296, and a CDRL3 of SEQ ID NO: 2297; (oo) the VH comprises a CDRH1 of SEQ ID NO: 1611, a CDRH2 of SEQ ID NO: 1612, and a CDRH3 of SEQ ID NO: 1613, and the VL comprises a CDRL1 of SEQ ID NO: 2298, a CDRL2 of SEQ ID NO: 2299, and a CDRL3 of SEQ ID NO: 2300; (pp) the VH comprises a CDRH1 of SEQ ID NO: 1614, a CDRH2 of SEQ ID NO: 1615, and a CDRH3 of SEQ ID NO: 1616, and the VL comprises a CDRL1 of SEQ ID NO: 2301, a CDRL2 of SEQ ID NO: 2302, and a CDRL3 of SEQ ID NO: 2303; (qq) the VH comprises a CDRH1 of SEQ ID NO: 1617, a CDRH2 of SEQ ID NO: 1618, and a CDRH3 of SEQ ID NO: 1619, and the VL comprises a CDRL1 of SEQ ID NO: 2304, a CDRL2 of SEQ ID NO: 2305, and a CDRL3 of SEQ ID NO: 2306; (rr) the VH comprises a CDRH1 of SEQ ID NO: 1626, a CDRH2 of SEQ ID NO: 1627, and a CDRH3 of SEQ ID NO: 1628, and the VL comprises a CDRL1 of SEQ ID NO: 2313, a CDRL2 of SEQ ID NO: 2314, and a CDRL3 of SEQ ID NO: 2315; (ss) the VH comprises a CDRH1 of SEQ ID NO: 1629, a CDRH2 of SEQ ID NO: 1630, and a CDRH3 of SEQ ID NO: 1631, and the VL comprises a CDRL1 of SEQ ID NO: 2316, a CDRL2 of SEQ ID NO: 2317, and a CDRL3 of SEQ ID NO: 2318; (tt) the VH comprises a CDRH1 of SEQ ID NO: 1632, a CDRH2 of SEQ ID NO: 1633, and a CDRH3 of SEQ ID NO: 1634, and the VL comprises a CDRL1 of SEQ ID NO: 2319, a CDRL2 of SEQ ID NO: 2320, and a CDRL3 of SEQ ID NO: 2321; (uu) the VH comprises a CDRH1 of SEQ ID NO: 1635, a CDRH2 of SEQ ID NO: 1636, and a CDRH3 of SEQ ID NO: 1637, and the VL comprises a CDRL1 of SEQ ID NO: 2322, a CDRL2 of SEQ ID NO: 2323, and a CDRL3 of SEQ ID NO: 2324; (vv) the VH comprises a CDRH1 of SEQ ID NO: 1638, a CDRH2 of SEQ ID NO: 1639, and a CDRH3 of SEQ ID NO: 1640, and the VL comprises a CDRL1 of SEQ ID NO: 2325, a CDRL2 of SEQ ID NO: 2326, and a CDRL3 of SEQ ID NO: 2327; (ww) the VH comprises a CDRH1 of SEQ ID NO: 1641, a CDRH2 of SEQ ID NO: 1642, and a CDRH3 of SEQ ID NO: 1643, and the VL comprises a CDRL1 of SEQ ID NO: 2328, a CDRL2 of SEQ ID NO: 2329, and a CDRL3 of SEQ ID NO: 2330; (xx) the VH comprises a CDRH1 of SEQ ID NO: 1644, a CDRH2 of SEQ ID NO: 1645, and a CDRH3 of SEQ ID NO: 1646, and the VL comprises a CDRL1 of SEQ ID NO: 2331, a CDRL2 of SEQ ID NO: 2332, and a CDRL3 of SEQ ID NO: 2333; (yy) the VH comprises a CDRH1 of SEQ ID NO: 1647, a CDRH2 of SEQ ID NO: 1648, and a CDRH3 of SEQ ID NO: 1649, and the VL comprises a CDRL1 of SEQ ID NO: 2334, a CDRL2 of SEQ ID NO: 2335, and a CDRL3 of SEQ ID NO: 2336; (zz) the VH comprises a CDRH1 of SEQ ID NO: 1650, a CDRH2 of SEQ ID NO: 1651, and a CDRH3 of SEQ ID NO: 1652, and the VL comprises a CDRL1 of SEQ ID NO: 2337, a CDRL2 of SEQ ID NO: 2338, and a CDRL3 of SEQ ID NO: 2339; (aaa) the VH comprises a CDRH1 of SEQ ID NO: 1653, a CDRH2 of SEQ ID NO: 1654, and a CDRH3 of SEQ ID NO: 1655, and the VL comprises a CDRL1 of SEQ ID NO: 2340, a CDRL2 of SEQ ID NO: 2341, and a CDRL3 of SEQ ID NO: 2342; (bbb) the VH comprises a CDRH1 of SEQ ID NO: 1656, a CDRH2 of SEQ ID NO: 1657, and a CDRH3 of SEQ ID NO: 1658, and the VL comprises a CDRL1 of SEQ ID NO: 2343, a CDRL2 of SEQ ID NO: 2344, and a CDRL3 of SEQ ID NO: 2345; or (ccc) the VH comprises a CDRH1 of SEQ ID NO: 1659, a CDRH2 of SEQ ID NO: 1660, and a CDRH3 of SEQ ID NO: 1661, and the VL comprises a CDRL1 of SEQ ID NO: 2346, a CDRL2 of SEQ ID NO: 2347, and a CDRL3 of SEQ ID NO: 2348.

Embodiment 106. The genetically engineered NK cell of any one of embodiments 103-105, wherein the second antigen recognition domain comprises a scFv comprising a VH and a VL, wherein: (a) the VH comprises SEQ ID NO: 82 and the VL comprises SEQ ID NO: 84; (b) the VH comprises SEQ ID NO: 21 and the VL comprises SEQ ID NO: 23; (c) the VH comprises SEQ ID NO: 31 and the VL comprises SEQ ID NO: 33; (d) the VH comprises SEQ ID NO: 41 and the VL comprises SEQ ID NO: 43; (e) the VH comprises SEQ ID NO: 51 and the VL comprises SEQ ID NO: 53; (f) the VH comprises SEQ ID NO: 61 and the VL comprises SEQ ID NO: 63; (g) the VH comprises SEQ ID NO: 693 and the VL comprises SEQ ID NO: 66; (h) the VH comprises SEQ ID NO: 694 and the VL comprises SEQ ID NO: 69; (i) the VH comprises SEQ ID NO: 695 and the VL comprises SEQ ID NO: 72; (j) the VH comprises SEQ ID NO: 74 and the VL comprises SEQ ID NO: 76; (k) the VH comprises SEQ ID NO: 78 and the VL comprises SEQ ID NO: 80; (1) the VH comprises SEQ ID NO: 11 and the VL comprises SEQ ID NO: 13; (m) the VH comprises SEQ ID NO: 92 and the VL comprises SEQ ID NO: 94; (n) the VH comprises SEQ ID NO: 102 and the VL comprises SEQ ID NO: 103; (o) the VH comprises SEQ ID NO: 104 and the VL comprises SEQ ID NO: 105; (p) the VH comprises SEQ ID NO: 712 and the VL comprises SEQ ID NO: 713; (q) the VH comprises SEQ ID NO: 714 and the VL comprises SEQ ID NO: 715; (r) the VH comprises SEQ ID NO: 716 and the VL comprises SEQ ID NO: 717; (s) the VH comprises SEQ ID NO: 718 and the VL comprises SEQ ID NO: 719; (t) the VH comprises SEQ ID NO: 720 and the VL comprises SEQ ID NO: 721; (u) the VH comprises SEQ ID NO: 722 and the VL comprises SEQ ID NO: 723; (v) the VH comprises SEQ ID NO: 724 and the VL comprises SEQ ID NO: 725; (w) the VH comprises SEQ ID NO: 948 and the VL comprises SEQ ID NO: 949; (x) the VH comprises SEQ ID NO: 950 and the VL comprises SEQ ID NO: 951; (y) the VH comprises SEQ ID NO: 952 and the VL comprises SEQ ID NO: 953; (z) the VH comprises SEQ ID NO: 954 and the VL comprises SEQ ID NO: 955; (aa) the VH comprises SEQ ID NO: 958 and the VL comprises SEQ ID NO: 959; (bb) the VH comprises SEQ ID NO: 960 and the VL comprises SEQ ID NO: 961; (cc) the VH comprises SEQ ID NO: 964 and the VL comprises SEQ ID NO: 965; (dd) the VH comprises SEQ ID NO: 966 and the VL comprises SEQ ID NO: 967; (ee) the VH comprises SEQ ID NO: 968 and the VL comprises SEQ ID NO: 969; (ff) the VH comprises SEQ ID NO: 970 and the VL comprises SEQ ID NO: 971; (gg) the VH comprises SEQ ID NO: 972 and the VL comprises SEQ ID NO: 973; (hh) the VH comprises SEQ ID NO: 974 and the VL comprises SEQ ID NO: 975; (ii) the VH comprises SEQ ID NO: 976 and the VL comprises SEQ ID NO: 977; (jj) the VH comprises SEQ ID NO: 980 and the VL comprises SEQ ID NO: 981; (kk) the VH comprises SEQ ID NO: 982 and the VL comprises SEQ ID NO: 983; (11) the VH comprises SEQ ID NO: 984 and the VL comprises SEQ ID NO: 985; (mm) the VH comprises SEQ ID NO: 990 and the VL comprises SEQ ID NO: 991; (nn) the VH comprises SEQ ID NO: 992 and the VL comprises SEQ ID NO: 993; (oo) the VH comprises SEQ ID NO: 994 and the VL comprises SEQ ID NO: 995; (pp) the VH comprises SEQ ID NO: 996 and the VL comprises SEQ ID NO: 997; (qq) the VH comprises SEQ ID NO: 998 and the VL comprises SEQ ID NO: 999; (rr) the VH comprises SEQ ID NO: 1000 and the VL comprises SEQ ID NO: 1001; (ss) the VH comprises SEQ ID NO: 1002 and the VL comprises SEQ ID NO: 1003; (tt) the VH comprises SEQ ID NO: 1004 and the VL comprises SEQ ID NO: 1005; (uu) the VH comprises SEQ ID NO: 1006 and the VL comprises SEQ ID NO: 1007; (vv) the VH comprises SEQ ID NO: 1008 and the VL comprises SEQ ID NO: 1009; (ww) the VH comprises SEQ ID NO: 1010 and the VL comprises SEQ ID NO: 1011; (xx) the VH comprises SEQ ID NO: 1016 and the VL comprises SEQ ID NO: 1017; (yy) the VH comprises SEQ ID NO: 1018 and the VL comprises SEQ ID NO: 1019; (zz) the VH comprises SEQ ID NO: 1020 and the VL comprises SEQ ID NO: 1021; (aaa) the VH comprises SEQ ID NO: 1022 and the VL comprises SEQ ID NO: 1023; (bbb) the VH comprises SEQ ID NO: 1024 and the VL comprises SEQ ID NO: 1025; (ccc) the VH comprises SEQ ID NO: 1026 and the VL comprises SEQ ID NO: 1027; (ddd) the VH comprises SEQ ID NO: 1028 and the VL comprises SEQ ID NO: 1029; (eee) the VH comprises SEQ ID NO: 1030 and the VL comprises SEQ ID NO: 1031; (fff) the VH comprises SEQ ID NO: 1032 and the VL comprises SEQ ID NO: 1033; (ggg) the VH comprises SEQ ID NO: 1034 and the VL comprises SEQ ID NO: 1035; (hhh) the VH comprises SEQ ID NO: 1036 and the VL comprises SEQ ID NO: 1037; or (iii) the VH comprises SEQ ID NO: 1038 and the VL comprises SEQ ID NO: 1039.

Embodiment 107. The method of any one of embodiments 103-106, wherein the second antigen recognition domain comprises a single domain antibody fragment, an adnectin peptide, an affibody, an afflilin, an affimer, an affitin, an alphabody, an anticalin, an avimer, a DARPin (Designed Ankyrin Repeat Protein), a Fynomer, a Kunitz domain peptide, a monobody, a centyrin, an aptamer, a T cell receptor (TCR)-like antibody, a single chain TCR (scTCR), or a portion of any of the foregoing.

Embodiment 108. The method of embodiment 103 or 104, wherein the second antigen recognition domain comprises a human CD27 extracellular domain.

Embodiment 109. The method of embodiment 108, wherein the human CD27 extracellular domain comprises the amino acid sequence of SEQ ID NO: 8

Embodiment 110. The method of embodiment 108 or 109, wherein the human CD27 extracellular domain comprises a mutation.

Embodiment 111. The method of embodiment 110, wherein the mutation reduces shedding of the human CD27 extracellular domain.

Embodiment 112. The genetically engineered NK cell of any one of embodiments 103-111, wherein the extracellular domain comprises a hinge.

Embodiment 113. The genetically engineered NK cell of embodiment 112, wherein the hinge comprises: (a) a portion of the extracellular region of CD8, CD8alpha, CD4, CD28, 4-1BB, or IgG; and/or (b) a human immunoglobulin CH2 region, a human immunoglobulin CH3 region, or both a human immunoglobulin CH2 region and a human immunoglobulin CH3 region.

Embodiment 114. The genetically engineered NK cell of embodiment 112 or 113, wherein the hinge comprises a human immunoglobulin CH2 region and wherein the human immunoglobulin CH2 region is an IgG1, IgG2 or IgG4 immunoglobulin CH2 region.

Embodiment 115. The genetically engineered NK cell of embodiment 112 or 113, wherein the hinge comprises a human immunoglobulin CH3 region and wherein the human immunoglobulin CH3 region is an IgG1, IgG2 or IgG4 immunoglobulin CH3 region.

Embodiment 116. The genetically engineered NK cell of any one of embodiments 103-115, wherein the transmembrane domain comprises a CD8, CD16, CD27, CD28, NKG2D, NKp44, NKp46, NKp30, NKp80, DNAM-1, CD3 zeta, CD3 epsilon, CD3 gamma, CD3 delta, CD45, CD4, CD5, CD9, CD22, CD33, CD37, CD64, CD80, CD86, CD134, CD137, CD154, ICOS/CD278, GITR/CD357, DAP10, DAP12 or erythropoietin receptor transmembrane domain, a portion of any of the foregoing, or a combination of any of the foregoing.

Embodiment 117. The genetically engineered NK cell of any one of embodiments 103-116, wherein the intracellular domain comprises a costimulatory domain.

Embodiment 118. The genetically engineered NK cell of any one of embodiments 103-117, wherein the intracellular domain comprises two or three costimulatory domains.

Embodiment 119. The genetically engineered NK cell of embodiment 117 or 118, wherein the costimulatory domain comprises a CD28, 4-1BB, DAP10, DAP12, 2B4, OX40, OX40L, ICOS, or CD27 costimulatory domain, or a portion of any of the foregoing.

Embodiment 120. The genetically engineered NK cell of any one of embodiments 103-119, wherein the intracellular domain comprises an activation domain.

Embodiment 121. The genetically engineered NK cell of embodiment 120, wherein the activation domain comprises a DAP12, FCER1G, FCGR2 Å, CD3zeta intracellular signaling domain, or a portion of any of the foregoing.

Embodiment 122. The genetically engineered NK cell of embodiment 121, wherein the activation domain comprises the CD3zeta intracellular signaling domain and the CD3zeta intracellular signaling domain comprises a mutation in an ITAM domain.

Embodiment 123. The genetically engineered NK cell of embodiment 122, wherein the mutation in the ITAM domain of the CD3zeta intracellular signaling domain comprises point mutations of each of the two tyrosine residues in one or more of the ITAM1, ITAM2, or ITAM3 domains to a phenylalanine residue.

Embodiment 124. The genetically engineered NK cell of embodiment 122, wherein the mutation in the ITAM domain of the CD3zeta activation domain comprises a deletion of one or more of the ITAM1, ITAM2, or ITAM3 domains.

Embodiment 125. The genetically engineered NK cell of any one of embodiments 103-124 further comprising an additional exogenous polypeptide.

Embodiment 126. The genetically engineered NK cell of embodiment 125, wherein the additional exogenous polypeptide comprises a cytokine, chemokine, ligand, receptor, monoclonal antibody, bispecific T cell engager, peptide or enzyme, a subunit or a portion of the foregoing, or any combination of the foregoing.

Embodiment 127. The genetically engineered NK cell of embodiment 126, wherein the additional exogenous polypeptide comprises a cytokine and wherein the cytokine comprises IL-15, membrane-bound IL-15 (mbIL-15), IL-2, membrane-bound IL-2, IL-12, membrane-bound IL-12, IL-18, membrane-bound IL-18, IL-21, membrane-bound IL-21, p40, LIGHT, CD40L, FLT3L, 4-1BBL, or FASL.

Embodiment 128. The genetically engineered NK cell of embodiment 125, wherein the additional exogenous polypeptide comprises IL-15RA or a fusion protein comprising IL-15 and IL-15RA.

Embodiment 129. The genetically engineered NK cell of embodiment 125, wherein the additional exogenous polypeptide is a tethered IL-21, a tethered IL-12, or a tethered IL-18.

Embodiment 130. The genetically engineered NK cell of any one of embodiments 125-129, further comprising a second additional exogenous polypeptide.

Embodiment 131. The genetically engineered NK cell of embodiment 129, wherein: (a) the first additional exogenous polypeptide is mbIL-15 and the second additional exogenous polypeptide is IL-15RA; or (b) the first additional exogenous polypeptide is soluble IL-15 and the second additional exogenous polypeptide is IL-15RA.

Embodiment 132. The genetically engineered NK cell of embodiment 125, wherein the additional exogenous polypeptide comprises a receptor and the receptor comprises CSF-1R, a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof.

Embodiment 133. The genetically engineered NK cell of embodiment 125, wherein the additional exogenous polypeptide is an enzyme and the enzyme comprises heparanase.

Embodiment 134. The genetically engineered NK cell of embodiment 125, wherein the additional exogenous polypeptide is a protein that overcomes immunosuppression of the tumor microenvironment.

Embodiment 135. The genetically engineered NK cell of embodiment 134, wherein the protein comprises a TGFbeta signal converter.

Embodiment 136. The genetically engineered NK cell of embodiment 135, wherein the TGFbeta signal converter comprises a TGFbeta receptor extracellular domain and an NK cell intracellular domain.

Embodiment 137. The genetically engineered NK cell of embodiment 136, wherein the NK cell intracellular domain comprises DAP10 or DAP12.

Embodiment 138. The genetically engineered NK cell of Embodiment 134, wherein the protein comprises a TGFbeta decoy receptor.

Embodiment 139. The genetically engineered NK cell of embodiment 125, wherein the additional exogenous polypeptide comprises a second CAR, comprising an antigen recognition domain that specifically binds an antigen other than human CD70.

Embodiment 140. The genetically engineered NK cell of embodiment 139, wherein the antigen other than human CD70 is CAIX, CD19, CD20, CD22, CD33, CD37, CD79a, CD79b, CD96, CD123, CD138, CLL-1, CXCR5, BCMA, FOLR2, FCRL5, FLT3, GPRC5D, HAVCR1, Her2, mesothelin, MUC16, EGFR, EGFRVIII, IL13Ra2, Trop2, GPC3, FOLR1, or GD2.

Embodiment 141. The genetically engineered NK cell of embodiment 125, wherein the additional exogenous polypeptide comprises a safety switch protein.

Embodiment 142. The genetically engineered NK cell of embodiment 125, wherein the genetically engineered NK cell comprises an additional exogenous polypeptide linked to the genetically engineered NK cell by chemical conjugation or by a sortase-mediated transpeptidation reaction.

Embodiment 143. The genetically engineered NK cell of any one of embodiments 86-142, wherein the genetically engineered NK has a reduced likelihood of fratricide by a NK cell expressing an anti-CD70 CAR compared to the likelihood of fratricide of a NK cell that has not been modified to one or more of: (a) a decreased level of total expressed CD70 polypeptide compared to the level of total expressed CD70 polypeptide in a wild-type NK cell; (b) a decreased level of surface expressed CD70 polypeptide compared to the level of surface expressed CD70 in a wild-type NK cell; (c) a decreased level of total expressed CD70 polypeptide compared to the level of total expressed CD70 polypeptide in a wild-type NK cell and comprise an anti-CD70 CAR; and (d) a decreased level of surface expressed CD70 polypeptide compared to the level of surface expressed CD70 in a wild-type NK cell and comprise an anti-CD70 CAR.

Embodiment 144. The genetically engineered NK cell of any one of embodiments 86-143, wherein the genetically engineered NK cell exhibits greater cell expansion rate thana NK cell that has not been modified to one or more of: (a) a decreased level of total expressed CD70 polypeptide compared to the level of total expressed CD70 polypeptide in a wild-type NK cell;

(b) a decreased level of surface expressed CD70 polypeptide compared to the level of surface expressed CD70 in a wild-type NK cell; (c) a decreased level of total expressed CD70 polypeptide compared to the level of total expressed CD70 polypeptide in a wild-type NK cell and comprise an anti-CD70 CAR; and (d) a decreased level of surface expressed CD70 polypeptide compared to the level of surface expressed CD70 in a wild-type NK cell and comprise an anti-CD70 CAR.

Embodiment 145. A population of cells, wherein at least about 30%, about 40%, about 50%, about 60%, about 70%, about 80%, about 90% or about 95% of the cells in the population are each the genetically engineered NK cell of any one of embodiments 86-144.

Embodiment 146. A pharmaceutical composition comprising the genetically engineered NK cell of any one of embodiments 84-124 or the population of embodiment 145, and a pharmaceutically acceptable carrier, diluent or excipient.

Embodiment 147. The pharmaceutical composition of Embodiment 146, wherein the pharmaceutical composition is cryopreserved.

Embodiment 148. The pharmaceutical composition of embodiment 147, wherein at least 10% of the NK cells in the cryopreserved pharmaceutical composition specifically bind human CD70 after thawing.

Embodiment 149. The pharmaceutical composition of any one of embodiments 146-148, wherein the pharmaceutical composition comprises from about 5×10⁵ NK cells to about 10×10¹² NK cells.

Embodiment 150. A method for treating a cancer in a subject, the method comprising administering to the subject an effective amount of the population of embodiment 145 or the pharmaceutical composition of any one of embodiment 146-148.

Embodiment 151. The method of embodiment 150, wherein the cancer is a CD70-positive cancer.

Embodiment 152. The method of embodiment 150 or 151, wherein the cancer is a solid tumor.

Embodiment 153. The method of embodiment 150 or 151, wherein the cancer is renal, lung, colorectal, ovarian, breast, head and neck, pancreatic, gastric, cervical, esophageal, or lung cancer, or glioblastoma.

Embodiment 154. The method of embodiment 150 or 151, wherein the cancer is a hematologic malignancy.

Embodiment 155. The method of embodiment 154, wherein the hematologic malignancy is acute myeloid leukemia (AML), non-Hodgkin's lymphoma (e.g., diffuse large B cell lymphoma (DLBCL), mantle cell lymphoma (MCL)), acute lymphoblastic leukemia, peripheral T-cell lymphoma (PTCL), anaplastic large cell lymphoma (ALCL), myelodysplastic syndrome (MDS), multiple myeloma, Waldenstrom's macroglobulinemia, or chronic lymphocytic leukemia (CLL).

Embodiment 156. The method of any one of embodiments 150-155, wherein the method further comprises administering an additional therapeutic agent.

Embodiment 157. The method of embodiment 156, wherein the additional therapeutic agent comprises an immune activator, a tyrosine kinase inhibitor, a metabolic inhibitor, an immune checkpoint inhibitor, a cytokine or a hypomethylating agent.

Embodiment 158. The method of embodiment 157, wherein the additional therapeutic agent is the immune activator, and the immune activator comprises 4-1BBL or OX-40.

Embodiment 159. The method of embodiment 157, wherein the additional therapeutic agent is the metabolic inhibitor and the metabolic inhibitor comprises an A2AR or IDO inhibitor.

Embodiment 160. The method of embodiment 157, wherein additional therapeutic agent is the checkpoint inhibitor and the checkpoint inhibitor comprises a PD-1, PD-L1, PD-L2, CTLA4, B7-H3, BTLA, KIR, LAG3, TIM-3, VISTA, AHR, c-cb1, or HPK1 inhibitor.

Embodiment 161. The method of embodiment 157, wherein the additional agent is the cytokine and the cytokine comprises IL-2, IL-15, IL-12, IL-18, or IL-21.

Embodiment 162. The method of embodiment 157, wherein the additional agent is the hypomethylating agent and the hypomethylating agent comprises azacitidine and/or decitabine.

Embodiment 163. The method of any one of embodiment s 150-162, wherein the population or pharmaceutical composition is administered to a human subject at a dose ranging from about 1×10⁵ NK cells per kg body weight to about 10×10⁹ NK cells per kg body weight.

1. A method of making a population of genetically engineered Natural Killer (NK) cells, the method comprising:
(a) contacting a population of NK cells with a CD70 inhibitor; and
(b) expanding the population of NK cells in vitro.

2. (canceled)

3. The method of claim 1, wherein the population of NK cells exhibits at least about 25% greater cell expansion compared to a population of NK cells that is not contacted with the CD70 inhibitor.

4. The method of claim 1, wherein the method further comprises, prior to step (a), isolating CD56⁺ cells and/or CD3⁻/CD56⁺ cells from a population of peripheral blood mononuclear cells (PBMCs) to obtain the population of NK cells.

5.-11. (canceled)

12. The method of claim 1, wherein the CD70 inhibitor comprises:
a small interfering RNA (siRNA) that targets CD70 mRNA, a short hairpin RNA (shRNA) that targets CD70 mRNA, a nucleic acid encoding a siRNA that targets CD70 mRNA, a nucleic acid encoding an shRNA that targets CD70 mRNA, a nucleic acid encoding a tandem shRNA that targets CD70 mRNA, a tandem shRNA that targets CD70 mRNA, a nucleic acid encoding a ribozyme that targets CD70 mRNA, a ribozyme that targets CD70 mRNA, or a combination of any of the foregoing;
an RNA-guided endonuclease and a guide RNA (gRNA) targeting a CD70 gene;
a Protein Expression Blocker (PEBL) or a nucleic acid encoding a PEBL, wherein the PEBL comprises a first antigen recognition domain that specifically binds human CD70 and one or more of a localizing domain, an intracellular retention domain and an endoplasmic reticulum (ER) retention domain; or
an antagonistic anti-CD70 antibody or an antigen-binding fragment thereof.

13.-19. (canceled)

20. The method of claim 1, further comprising:
(c) contacting the population of NK cells with a polynucleotide encoding a chimeric antigen receptor (CAR) under conditions sufficient to transfer the polynucleotide across a cell membrane of at least one NK cell in the population of NK cells, wherein the CAR comprises:
(i) an extracellular domain comprising a second antigen recognition domain that specifically binds human CD70;
(ii) a transmembrane domain; and
(iii) an intracellular domain.

21.-32. (canceled)

33. The method of claim 1, further comprising:
(e) contacting the population of NK cells with at least one polynucleotide encoding at least one exogenous polypeptide.

34.-49. (canceled)

50. A genetically engineered natural killer (NK) cell modified to have:
a) a decreased level of total expressed CD70 polypeptide compared to the level of total expressed CD70 polypeptide in a wild-type NK cell, and/or
b) a decreased level of surface expressed CD70 polypeptide compared to the level of surface expressed CD70 in a wild-type NK cell.

51.-52. (canceled)

53. The genetically engineered NK cell of claim 50, wherein the genetically engineered NK cell comprises at least about 30% less of surface expressed CD70 polypeptide and/or total expressed CD70 polypeptide than the wild-type NK cell.

54. The genetically engineered NK cell of claim 50, wherein the level of CD70 mRNA in the genetically engineered NK cell is reduced as compared to the level of CD70 mRNA in a wild-type NK cell.

55. The genetically engineered NK cell of claim 50, wherein the genetically engineered NK cell comprises:
a siRNA that targets CD70 mRNA, a nucleic acid encoding a siRNA that targets CD70 mRNA, a shRNA that targets CD70 mRNA, a nucleic acid encoding a shRNA that targets CD70 mRNA, a nucleic acid encoding a tandem shRNA that targets CD70 mRNA, a tandem shRNA that targets CD70 mRNA, a nucleic acid encoding a ribozyme that targets CD70 mRNA, or a ribozyme that targets CD70 mRNA, or a combination of any of the foregoing;
an RNA guided endonuclease and a gRNA targeting a CD70 gene; or
a PEBL or a nucleic acid encoding a PEBL, wherein the PEBL comprises a first antigen recognition domain that specifically binds human CD70 and one or more of a localizing domain, an intracellular retention domain and an ER retention domain.

56.-57. (canceled)

58. The genetically engineered NK cell of claim 50, wherein the genetically engineered NK cell is derived from umbilical cord blood cells, PBMCs, mobilized unstimulated leukapheresis products (PBSCs), unmobilized PBSCs, human embryonic stem cells (hESCs), induced pluripotent stem cells (iPSCs), mesenchymal stem cells (MSCs), hematopoietic stem cells (HSCs), bone marrow, or CD34⁺ cells.

59. (canceled)

60. The genetically engineered NK cell of claim 50, wherein the genetically engineered NK cell comprises a CAR and/or a polynucleotide encoding the CAR, wherein the CAR comprises:
(a) an extracellular domain comprising a second antigen recognition domain that specifically binds human CD70;
(b) a transmembrane domain; and
(c) an intracellular domain.

61.-70. (canceled)

71. The genetically engineered NK cell of claim 50 further comprising at least one exogenous polypeptide.

72. The genetically engineered NK cell of claim 71, wherein the at least one exogenous polypeptide comprises a cytokine, chemokine, ligand, receptor, monoclonal antibody, bispecific T cell engager, peptide or enzyme, a subunit or a portion of the foregoing, or any combination of the foregoing.

73. The genetically engineered NK cell of claim 72, wherein the at least one exogenous polypeptide comprises a cytokine and wherein the cytokine comprises IL-15, membrane-bound IL-15 (mbIL-15), IL-2, membrane-bound IL-2, IL-12, membrane-bound IL-12, IL-18, membrane-bound IL-18, IL-21, membrane-bound IL-21, p40, LIGHT, CD40L, FLT3L, 4-1BBL, or FASL.

74. The genetically engineered NK cell of claim 71, wherein the at least one exogenous polypeptide comprises IL-15RA, IL-15, or is a fusion protein comprising IL-15 and IL-15RA.

75. (canceled)

76. The genetically engineered NK cell of claim 71, further comprising a first exogenous polypeptide comprising mbIL-15 and a second exogenous polypeptide comprising IL-15RA.

77. The genetically engineered NK cell of claim 71, wherein the at least one exogenous polypeptide comprises a receptor selected from the group consisting of: CSF-1R, a CXC chemokine receptor, a CC chemokine receptor, a CX3C chemokine receptor, a XC chemokine receptor, or a chemokine-binding fragment thereof.

78. (canceled)

79. The genetically engineered NK cell of claim 71, wherein the at least one exogenous polypeptide comprises a TGFbeta signal converter.

80. The genetically engineered NK cell of claim 79, wherein the TGFbeta signal converter comprises a TGFbeta receptor extracellular domain and an NK cell intracellular domain.

81. The genetically engineered NK cell of claim 71, wherein the at least one exogenous polypeptide comprises a TGFbeta decoy receptor comprising a TGFbeta receptor extracellular domain and optionally, a transmembrane domain.

82.-83. (canceled)

84. The genetically engineered NK cell of claim 71, wherein the at least one exogenous polypeptide comprises a CAR comprises at least one antigen recognition domain that specifically binds to an antigen other than human CD70.

85.-87. (canceled)

88. The genetically engineered NK cell of claim 50, wherein the genetically engineered NK has a reduced likelihood of fratricide by a NK cell expressing an anti-CD70 CAR compared to the likelihood of fratricide of a wild-type NK cell.

89. The genetically engineered NK cell of claim 50, wherein the genetically engineered NK cell exhibits greater fold cell expansion that a wildtype NK cell.

90. A population of cells, wherein at least about 30% of cells in the population are the genetically engineered NK cell of claim 50.

91. A pharmaceutical composition comprising the genetically engineered NK cell of claim 50, and a pharmaceutically acceptable carrier, diluent, or excipient.

92. A method for treating a cancer in a subject, the method comprising administering to the subject an effective amount of the pharmaceutical composition of claim 91.

93.-98. (canceled)
